(19) United States
(12) Patent Application Publication
(10) Pub. No.: US 2026/0199386 A1
(43) Pub. Date: Jul. 16, 2026

(54) SYNTHETIC SINGLE-STRANDED DNA MOLECULES AND METHODS OF PRODUCING AND USING THE SAME

(71) Applicant: Generation Bio Co., Cambridge, MA (US)

(72) Inventors: Russell Monds, Cambridge, MA (US); Kelly Ann Miller, Cambridge, MA (US); Anthony Rohit Dawson, Salem, MA (US); Daniel Jason Blackstock, Walpole, MA (US)

(21) Appl. No.: 19/134,114

(22) PCT Filed: Dec. 1, 2023
(86) PCT No.: PCT/US2023/082143

§ 371 Date: May 29, 2025

Related U.S. Application Data
(60) Provisional application No. 63/429,461, filed on Dec. 1, 2022.
(60) Provisional application No. 63/449,872, filed on Mar. 3, 2023.
(60) Provisional application No. 63/529,637, filed on Jul. 28, 2023.
(60) Provisional application No. 63/544,571, filed on Oct. 17, 2023.

(51) Int. Cl. A61K 31/7088; A61K 9/51; A61K 38/00; A61K 48/00; C12N 9/12; C12N 9/22; C12N 15/10; C12N 15/113; C12P 19/34

(52) U.S. Cl. CPC A61K 31/7088; A61K 9/5123; A61K 38/00; A61K 48/005; C12N 9/1241; C12N 9/22; C12N 15/1003; C12N 15/113; C12P 19/34; C12Y 301/11003; C12Y 301/21004; C12N 2310/15; C12N 2310/315; C12N 2310/333; C12N 2310/336; C12N 2310/531; C12N 2830/34

(57) ABSTRACT

The present application discloses modified single-stranded DNA molecules, as well as their cell-free methods of synthesis and their use as therapeutic agents.

### RELATED APPLICATIONS

The instant application claims priority to U.S. Provisional Application No. 63/429,461, filed Dec. 1, 2022; U.S. Provisional Application No. 63/449,872, filed Mar. 3, 2023; U.S. Provisional Application No. 63/529,637, filed Jul. 28, 2023; and U.S. Provisional Application No. 63/544,571, filed Oct. 17, 2023. The entire contents of each of the foregoing applications are expressly incorporated by reference herein.

### BACKGROUND

Vectors derived from adeno-associated viruses AAV (i.e., recombinant AAV (rAAV) or AAV vectors) are attractive for delivering genetic material because (i) they are able to infect (“transduce”) a wide variety of dividing as well as non-dividing cell types such as myocytes and neurons; (ii) they are devoid of the virus structural genes, thereby diminishing the host cell responses to virus infection, e.g., interferon-mediated responses; (iii) wild-type AAVs are considered non-pathologic in humans; and (iv) in contrast to wild type AAVs, which are capable of integrating into the host cell genome, replication-deficient AAV vectors lack the rep gene and generally persist as an episome, thus greatly limiting the risk of insertional mutagenesis or genotoxicity.

However, there are several major drawbacks and deficiencies in using AAV particles as a gene delivery vector that stems from conventional AAV production from host cells (e.g., Sf9 insect cells in a high scale production setting). One major drawback associated with rAAV is its limited viral packaging capacity of about 4.5 kb of heterologous DNA (Dong et al., 1996; Athanasopoulos et al., 2004; Lai et al., 2010). As a result, the use of AAV vectors has been limited to less than 150 kDa protein coding capacity due to this limitation in viral packaging. A second drawback is related to the capsid immunogenicity that prevents re-administration to patients. The immune system in the patients can respond to the vector which effectively acts as a booster to stimulate the immune system generating high titer anti-AAV antibodies that preclude future treatments. Some recent reports indicate concerns with immunogenicity in high dose situations. Another notable drawback is that production of AAV in host cells (e.g., insect cells) in a high scale for the manufacture of the viral genome result in a random mixture of plus (+) and minus (−) stranded vectors. This drastically decreases the strand specificity of a transgene for the much-needed therapeutic expression of the sense strand.

Additionally, conventional AAV virions with capsids are produced by introducing a plasmid or plasmids containing the AAV genome, rep genes, and cap genes (Grimm et al., 1998). However, such encapsidated AAV virus vectors were found to inefficiently transduce certain cell and tissue types and the capsids were also found to induce a severe immune response in hosts. Accordingly, use of adeno-associated virus (AAV) vectors for gene therapy (including gene editing) is limited to single administration to patients due to the patient immune response, the limited range of transgene genetic material suitable for delivery in AAV vectors due to minimal viral packaging capacity (about 4.5 kb), and slow AAV-mediated gene expression. Further, the methods of producing such AAV vectors have relied greatly upon traditional insect cell dependent production methods. Such methods can be stymied by contaminants from the cells used to produce the vectors which are inconvenient or costly to remove or purify away, and which may pose undesirable side effects if included in a therapeutic formulation.

Accordingly, there is a strong need in the gene therapy field for a technology that is minimally immunogenic, re-dosable, and allows for the generation of recombinant vectors in large quantity, that also increases expression level, strand specificity, and purity while increasing the capacity of a transgene size.

### SUMMARY

The technology described herein is directed in general to novel single-stranded deoxyribonucleic acid (ssDNA) molecules (e.g., single-stranded DNA), as well as methods for generating single-stranded DNA molecules, e.g., in the absence of cells or cell lines. As such, the resulting single-stranded DNA molecules have fewer impurities than comparable vectors made using conventional cell-based production methodologies and exhibit significantly lower immunogenicity in mammalian hosts, which may translate into better in vivo expression that is sustained a longer duration of time after administration. According to some aspects, the disclosure features cell free, synthetic methods of single-stranded DNA molecules using rolling circle amplification and enzymatic degradation.

In a first aspect, the disclosure provides a method for producing a linear, single-stranded DNA (ssDNA) molecule comprising at least one nucleic acid sequence of interest flanked by at least one stem-loop structure comprising at least one stem and at least one loop at the 3′ end, the method comprising the sequential steps of (a) contacting a double-stranded, closed-ended DNA (ceDNA) molecule comprising the at least one nucleic acid sequence of interest with an endonuclease; (b) contacting the double-stranded ceDNA with an exonuclease, thereby producing the linear, ssDNA molecule. According to some embodiments, the ceDNA molecule further comprises at least one promoter. According to some embodiments, the promoter comprises a transcription start site (TSS).

According to some embodiments of the aspects and embodiments herein, the ceDNA molecule further comprises at least one enhancer. According to some embodiments of the aspects and embodiments herein, the promoter is double-stranded in the ssDNA molecule. According to some embodiments of the aspects and embodiments herein, the TSS is double-stranded in the ssDNA molecule. According to some embodiments of the aspects and embodiments herein, the enhancer is double-stranded in the ssDNA molecule. According to some embodiments of the aspects and embodiments herein, the ssDNA molecule further comprises at least one stem-loop structure comprising at least one stem and one loop at the 5′ end. According to some embodiments of the aspects and embodiments herein, the at least one stem-loop structure at the 3′ end comprises at least two stem-loop structures, and/or wherein the at least one stem-loop structure at the 5′ end comprises at least two stem-loop structures. According to further embodiments, the ceDNA molecule comprises one or more endonuclease recognition sequences. According to some embodiments of the aspects and embodiments herein, the stem loop structure at the 3′ end comprises one or more endonuclease recognition sequences. According to some embodiments of the aspects and embodiments herein, the stem loop structure at the 5′ end comprises one or more endonuclease recognition sequences.

According to some embodiments of the aspects and embodiments herein, the one or more endonuclease recognition sequences are selected from the group consisting of: 5′-CCAA-3′ (Nb.BtsI) (Nb.BsrDI) (Nt.CviPII), 5′-CCAAGC-3′ (Nb.BbvCI), 5′-CCAACC-3′ (Nb.BbvCI), 5′-CCAAGAGTCNNNN-3′ (Nt.BstNBI)-N can be A, G, C or T, 5′-CCAAG-3′ (Nb.BsmI), 5′-CCAAC-3′ (Nb.BssSI), 5′-CCAAGGATCNNNN-3′ (Nt.AlwI), CCAAGTCTCN-3′ (Nt.BsmAI), and CCAAGCTCTTCN-3′ (Nt.BspQI). According to some embodiments of the aspects and embodiments herein, the terminal residue of the stem-loop structure at the 3′ end is capable of priming replication and/or transcription inside the nucleus of a host cell. According to some embodiments, the 3′ terminal residue comprises a free-OH.

According to some embodiments of the aspects and embodiments herein, the contacting the double-stranded ceDNA molecule with the endonuclease creates one or more nicks in a sense strand of said nucleic acid sequence of interest, thereby creating a nicked ceDNA molecule. According to further embodiments, the one or more nicks in the sense strand of the nucleic acid sequence of interest are located 5′ upstream of the nucleic acid sequence of interest, within the nucleic acid sequence of interest, and/or 3′ upstream of the nucleic acid sequence of interest. According to other further embodiments, the one or more nicks in the sense strand of the nucleic acid sequence of interest are located 5′ upstream of the nucleic acid sequence of interest. According to still other further embodiments of the aspects and embodiments herein, the one or more nicks in the sense strand of the nucleic acid sequence of interest are located 3′ downstream of the nucleic acid sequence of interest. According to some embodiments of the aspects and embodiments herein, the one or more nicks in the sense strand of the nucleic acid sequence of interest are located within the nucleic acid sequence of interest.

According to some embodiments of the aspects and embodiments herein, the sense strand further comprises at least one phosphorothioate (PS) modified nucleotide downstream of said expression cassette. According to some embodiments of the aspects and embodiments herein, the sense strand further comprises at least 2 PS modified nucleotides downstream of said expression cassette. According to some embodiments of the aspects and embodiments herein, the sense strand further comprises at least 3 PS modified nucleotides downstream of said expression cassette. According to some embodiments of the aspects and embodiments herein, the sense strand further comprises at least 4 PS modified nucleotides downstream of said expression cassette. According to some embodiments of the aspects and embodiments herein, the sense strand further comprises at least 5 PS modified nucleotides downstream of said expression cassette. According to some embodiments of the aspects and embodiments herein, the sense strand further comprises at least one phosphorothioate (PS) modified nucleotide upstream of said expression cassette. According to some embodiments of the aspects and embodiments herein, the sense strand further comprises at least 2 PS modified nucleotides upstream of said expression cassette. According to some embodiments of the aspects and embodiments herein, the sense strand further comprises at least 3 PS modified nucleotides upstream of said expression cassette. According to some embodiments of the aspects and embodiments herein, the sense strand further comprises at least 4 PS modified nucleotides upstream of said expression cassette. According to some embodiments of the aspects and embodiments herein, the sense strand further comprises at least 5 PS modified nucleotides upstream of said expression cassette.

According to some embodiments of the aspects and embodiments herein, the contacting the nicked ceDNA molecule with an exonuclease creates a stretch of single-stranded DNA (ssDNA) corresponding to the nucleic acid sequence of interest in the double-stranded ceDNA molecule. According to some embodiments of the aspects and embodiments herein, the endonuclease is a Type II restriction enzyme. According to some embodiments of the aspects and embodiments herein, the endonuclease is selected from group consisting of Nb.BtsI, Nb.BsrDI, Nt.CviPII, Nb.BbvC1, Nt.BbvCI, Nt.BstNBI, Nb.BsmI, Nb.BssSI, Nt.AlwI, Nt.BsmA1, Nt.BspQI, and Endonuclease V (Endo V). According further embodiments, the Type II restriction enzyme is Nb.BbvCI. According to other further embodiments, the endonuclease is Endo V. According to some embodiments of the aspects and embodiments herein, the double-stranded ceDNA molecule comprises at least one deoxyinosine residue. According to some embodiments, the deoxyinosine residue is present in the at least one stem-loop structure at the 3′ end, two bases upstream of a desired nick site. According to some embodiments of the aspects and embodiments herein, the double-stranded ceDNA molecule comprises at least one uridine-, inosine-, xanthosine-, and/or oxanosine-containing residue that is nicked by the endonuclease, wherein the endonuclease has enzymatic activity on the uridine-, inosine-, xanthosine-, and/or oxanosine-containing residue. According to some embodiments, the endonuclease nicks the DNA at the second phosphodiester bond 3′ to uridine-, inosine-, xanthosine-, and/or oxanosine-containing residue.

According to some embodiments of the aspects and embodiments herein, the exonuclease is a T7 exonuclease. According to some embodiments of the aspects and embodiments herein, the exonuclease is Exonuclease III (Exo III). According to some embodiments of the aspects and embodiments herein, the method further comprises the steps of (1) performing rolling circle amplification (RCA) using a double-stranded DNA (dsDNA) molecule, thereby producing an intermediate dsDNA molecule; and (2) performing cell-free, enzymatic synthesis using the intermediate dsDNA molecule, thereby producing the ceDNA molecule, wherein steps (1) and (2) are performed prior to steps (a) and (b). According to some embodiments, the method further comprises a step of (3) purifying the ceDNA molecule after step (2) and prior to step (a). According to some embodiments of the aspects and embodiments herein, the RCA step (1) comprises the step of (i) contacting the dsDNA molecule with a primer and a DNA polymerase. According to some embodiments of the aspects and embodiments herein, the step (2) comprises the steps of (i) contacting the intermediate dsDNA molecule with a restriction endonuclease to produce a cleaved intermediate dsDNA molecule, (ii) contacting the cleaved intermediate dsDNA molecule with an oligonucleotide comprising an end compatible with at least one end the cleaved intermediate dsDNA molecule and a ligase. According to a further embodiment, step (ii) further comprises contacting the cleaved intermediate dsDNA molecule with at least two oligonucleotides each comprising ends compatible with at least one end of the cleaved intermediate dsDNA molecule. According to another further embodiment, the at least two oligonucleotides each comprise the same end. According to another further embodiment, the at least two oligonucleotides each comprise different ends. According to some embodiments of the aspects and embodiments herein, the at least two oligonucleotides are the same. According to some embodiments of the aspects and embodiments herein, the at least two oligonucleotides are different. According to some embodiments of the aspects and embodiments herein, step (2) further comprises a step of (iii) ligating the at least one oligonucleotide to the cleaved dsDNA intermediate.

According to some embodiments of the aspects and embodiments herein, the at least one stem at the 3′ end comprises a partial DNA duplex of between 4-500 nucleotides. According to some embodiments of the aspects and embodiments herein, the at least one stem at the 3′ end comprises a partial DNA duplex of 4-5 nucleotides. According to some embodiments of the aspects and embodiments herein, the at least one stem at the 5′ end comprises a partial DNA duplex of between 4-500 nucleotides, for example, 4-10, 4-20, 4-30, 4-40, 4-50, 4-100, 4-200, 4-300, 4-400, 4-500, 10-500, 20-500, 50-500, 100-500, 200-500, 300-500, 400-500, 10-20, 10-30, 20-40, 10-50, 10-100, 10-200, 10-300, 10-400, 20-50, 50-100, 100-200, 200-300, 300-400, 400-500, 25-50, 50-75, 75-100, 150-175, 200-250, 300-350, 400-450, or 450-500. According to some embodiments of the aspects and embodiments herein, the at least one stem at the 5′ end comprises a partial DNA duplex of 4-5 nucleotides. According to some embodiments of the aspects and embodiments herein, the at least one loop at the 3′ end comprises between 3-500 unbound nucleotides. According to some embodiments of the aspects and embodiments herein, the at least one loop at the 3′ end comprises a minimum of 3 unbound nucleotides. According to some embodiments of the aspects and embodiments herein, the at least one loop at the 5′ end comprises between 3-500 unbound nucleotides, for example 3, 4, 5, 6, 7, 8, 9, 10, 15, 20, 25, 30, 35, 40, 45, 50, 75, 100, 150, 200, 250, 300, 350, 400, 450, or 500 unbound nucleotides. According to some embodiments of the aspects and embodiments herein, the at least one loop at the 5′ end comprises a minimum of 3 unbound nucleotides.

According to some embodiments of the aspects and embodiments herein, the ssDNA comprises at least two stem-loop structures at the 3′ end. According to some embodiments of the aspects and embodiments herein, the ssDNA comprises at least three stem-loop structures at the 3′ end. According to some embodiments of the aspects and embodiments herein, the ssDNA comprises at least four or more stem-loop structures at the 3′ end. According to some embodiments of the aspects and embodiments herein, the ssDNA comprises at least two stem-loop structures at the 3′ end. According to some embodiments of the aspects and embodiments herein, the ssDNA comprises at least three stem-loop structures at the 3′ end. According to some embodiments of the aspects and embodiments herein, the ssDNA comprises at least four or more stem-loop structures at the 3′ end. According to some embodiments of the aspects and embodiments herein, the ssDNA comprises at least one bubble structure at the 5′ end. According to some embodiments of the aspects and embodiments herein, the ssDNA comprises at least two stem-loop structures at the 5′ end. According to some embodiments of the aspects and embodiments herein, the ssDNA comprises at least three stem-loop structures at the 5′ end. According to some embodiments of the aspects and embodiments herein, the ssDNA comprises at least four or more stem-loop structures at the 5′ end. According to some embodiments of the aspects and embodiments herein, the at least one stem-loop structure at the 3′ end comprises a hairpin DNA structure. According to some embodiments of the aspects and embodiments herein, the at least one stem-loop structure at the 3′ end comprises a DNA structure selected from the group consisting of: a cruciform DNA structure, a hammerhead DNA structure, a quadraplex DNA structure, a bulged DNA structure, a multibranched loop structure, and a bubble structure.

According to some embodiments of the aspects and embodiments herein, the at least one stem-loop structure at the 3′ end does not comprise the A or A′ regions that would be present in a wild-type AAV ITR. According to some embodiments of the aspects and embodiments herein, the at least one stem-loop structure at the 3′ end does not comprise the A, A′, D, or D′ regions that would be present in a wild-type AAV ITR. According to some embodiments of the aspects and embodiments herein, the at least one stem-loop structure at the 3′ end does not comprise the A, A′, B, B′, C, C′, D, or D′ regions that would be present in a wild-type AAV ITR. According to some embodiments of the aspects and embodiments herein, the at least one stem-loop structure at the 5′ end does not comprise the A or A′ regions that would be present in a wild-type AAV ITR. According to some embodiments of the aspects and embodiments herein, the at least one stem-loop structure at the 5′ end does not comprise the A, A′, D, or D′ regions that would be present in a wild-type AAV ITR. According to some embodiments of the aspects and embodiments herein, the at least one stem-loop structure at the 5′ end does not comprise the A, A′, B, B′, C, C′, D, or D′ regions that would be present in a wild-type AAV ITR. According to some embodiments of the aspects and embodiments herein, the at least one stem-loop structure at the 3′ end does not comprise a rep binding element (RBE) that would be present in a wild-type AAV ITR. According to some embodiments of the aspects and embodiments herein, the at least one stem-loop structure at the 3′ end does not comprise a terminal resolution site (trs) that would be present in a wild-type AAV ITR. According to some embodiments of the aspects and embodiments herein, the at least one stem-loop structure at the 5′ end does not comprise a rep binding element (RBE) that would be present in a wild-type AAV ITR. According to some embodiments of the aspects and embodiments herein, the at least one stem-loop structure at the 5′ end does not comprise a terminal resolution site (trs) that would be present in a wild-type AAV ITR. According to some embodiments of the aspects and embodiments herein, the ssDNA molecule does not comprise any virally-derived sequences.

According to some embodiments of the aspects and embodiments herein, the at least one stem-loop structure at the 3′ end comprises one or more nucleotides that are modified to be exonuclease resistant. According to some embodiments, the nucleotides that are modified to be exonuclease resistant are selected from the group consisting of phosphorothioate-modified nucleotides, locked nucleic acid (LNA)-modified nucleotides, 2′-O-methyl (m)-modified nucleotides, 2′-O-methoxy ethyl (E)-modified nucleotides, 2′-fluoro (F)-modified nucleotides, and combinations thereof. According to some embodiments of the aspects and embodiments herein, the at least one stem-loop structure at the 3′ end and/or the at least one stem-loop structure at the 5′ end each independently comprise a functional moiety. According to some embodiments of the aspects and embodiments herein, the at least one stem-loop structure at the 5′ end comprises a hairpin DNA structure. According to some embodiments of the aspects and embodiments herein, the at least one stem-loop structure at the 5′ end comprises a DNA structure selected from the group consisting of: a cruciform DNA structure, a hammerhead DNA structure, a quadraplex DNA structure, a bulged DNA structure, a multibranched loop structure, and a bubble structure.

According to some embodiments of the aspects and embodiments herein, the stem structure at the 5′ end comprises one or more nucleotides that are modified to be exonuclease resistant. According to some embodiments of the aspects and embodiments herein, the nucleotides that are modified to be exonuclease resistant are PS modified nucleotides. According to some embodiments of the aspects and embodiments herein, the at least one loop structure at the 5′ end further comprises one or more nucleic acids to stabilize the ends. According to some embodiments of the aspects and embodiments herein, the at least one loop structure at the 5′ end further comprises one or more nucleic acids that are chemically modified. According to some embodiments of the aspects and embodiments herein, the deoxyinosine residue is present at the position of -1i, -2i, -5i, or -7i relative to SEQ ID NO: 7. According to some embodiments of the aspects and embodiments herein, the deoxyinosine residue is present at the position of -1i or -7i relative to SEQ ID NO: 7.

According to some embodiments of the aspects and embodiments herein, the ssDNA molecule is capable of being transported across the nuclear membrane from the cytosol into the nucleus of a host cell. According to some embodiments of the aspects and embodiments herein, the ssDNA molecule further comprises at least one functional moiety. According to some embodiments of the aspects and embodiments herein, the at least one stem loop structure at the 3′ end comprises at least one functional moiety. According to some embodiments of the aspects and embodiments herein, the at least one stem-loop structure at the 5′ end comprises at least one functional moiety. According to some embodiments of the aspects and embodiments herein, the at least one functional moiety is an aptamer. According to some embodiments of the aspects and embodiments herein, the loops at the 5′ and/or 3′ ends further comprise one or more aptamers. According to some embodiments of the aspects and embodiments herein, the aptamer is encoded in the ceDNA molecule, and wherein the aptamer forms a secondary aptamer structure in the ssDNA molecule. According to some embodiments of the aspects and embodiments herein, the aptamer is a CH4-1 aptamer. According to some embodiments of the aspects and embodiments herein, the at least one loop at the 3′ and/or 5′ ends further comprise one or more synthetic ribozymes. According to some embodiments of the aspects and embodiments herein, the at least one loop at the 3′ and/or 5′ ends further comprise one or more antisense oligonucleotides (ASOs). According to some embodiments of the aspects and embodiments herein, the at least one loop at the 3′ and/or 5′ ends further comprise one or more short-interfering RNAs (siRNAs). According to some embodiments of the aspects and embodiments herein, the at least one loop at the 3′ and/or 5′ ends further comprise one or more antiviral nucleoside analogues (ANAs). According to some embodiments of the aspects and embodiments herein, the at least one loop at the 3′ and/or 5′ ends further comprise one or more triplex forming oligonucleotides. According to some embodiments of the aspects and embodiments herein, the at least one loop at the 3′ and/or 5′ ends further comprise one or more gRNAs or gDNAs. According to some embodiments of the aspects and embodiments herein, the at least one loop at the 3′ and/or 5′ ends further comprise one or more molecular probes. According to some embodiments of the aspects and embodiments herein, the ssDNA molecule is devoid of any viral capsid protein coding sequences. According to some embodiments of the aspects and embodiments herein, the ssDNA molecule comprises a first ITR and a second ITR, and wherein the ITRs do not comprise any virally derived sequences.

According to some embodiments of the aspects and embodiments herein, the ssDNA molecule does not comprise any virally-derived sequences. According to some embodiments of the aspects and embodiments herein, the ssDNA molecule comprises a first ITR and a second ITR, and wherein the ITRs are synthetic. According to some embodiments of the aspects and embodiments herein, the ssDNA molecule is synthetically produced in vitro. According to some embodiments of the aspects and embodiments herein, the ssDNA molecule is synthetically produced in vitro in a cell-free environment. According to some embodiments of the aspects and embodiments herein, the ssDNA molecule does not activate or minimally activates an immune pathway. According to some embodiments, the immune pathway is an innate immune pathway. According to other further embodiments, the immune pathway is an innate immune pathway selected from the group consisting of the cGAS/STING pathway, the TLR9 pathway, an inflammasome-mediated pathway, and combinations thereof. According to some embodiments of the aspects and embodiments herein, the nucleic acid sequence of interest is a therapeutic protein or a therapeutic fragment thereof.

According to some embodiments of the aspects and embodiments herein, the at least one therapeutic protein is selected from the group consisting of an antibody, an enzyme, a coagulation factor, a transcription factor, a replication factor, a growth factor, a hormone, and a fusion protein. According to further embodiments, the at least one therapeutic protein is useful for treating a genetic disorder selected from the group consisting of melanoma, hemophilia A (clotting factor VIII (FVIII) deficiency) and hemophilia B (clotting factor IX (FIX) deficiency), cystic fibrosis (CFTR), familial hypercholesterolemia (LDL receptor defect), hepatoblastoma, Wilson disease, phenylketonuria (PKU), congenital hepatic porphyria, inherited disorders of hepatic metabolism, Lesch Nyhan syndrome, sickle cell anemia, thalassaemias, xeroderma pigmentosum, Fanconi's anemia, retinitis pigmentosa, ataxia telangiectasia, Bloom's syndrome, retinoblastoma, mucopolysaccharide storage diseases (e.g., Hurler syndrome (MPS Type I), Scheie syndrome (MPS Type I S), Hurler-Scheie syndrome (MPS Type I H-S), Hunter syndrome (MPS Type II), Sanfilippo Types A, B, C, and D (MPS Types III A, B, C, and D), Morquio Types A and B (MPS IVA and MPS IVB), Maroteaux-Lamy syndrome (MPS Type VI), Sly syndrome (MPS Type VII), hyaluronidase deficiency (MPS Type IX)), Niemann-Pick Disease Types A/B, C1 and C2, Fabry disease, Schindler disease, GM2-gangliosidosis Type II (Sandhoff Disease), Tay-Sachs disease, Metachromatic Leukodystrophy, Krabbe disease, Mucolipidosis Type I, II/III and IV, Sialidosis Types I and II, Glycogen Storage disease Types I and II (Pompe disease), Gaucher disease Types I, II and III, Fabry disease, cystinosis, Batten disease, Aspartylglucosaminuria, Salla disease, Danon disease (LAMP-2 deficiency), Lysosomal Acid Lipase (LAL) deficiency, neuronal ceroid lipofuscinoses (CLN1-8, INCL, and LINCL), sphingolipidoses, galactosialidosis, amyotrophic lateral sclerosis (ALS), Parkinson's disease, Alzheimer's disease, Huntington's disease, spinocerebellar ataxia, spinal muscular atrophy, Friedreich's ataxia, Duchenne muscular dystrophy (DMD), Becker muscular dystrophies (BMD), dystrophic epidermolysis bullosa (DEB), ectonucleotide pyrophosphatase 1 deficiency, generalized arterial calcification of infancy (GACI), Leber Congenital Amaurosis, Stargardt macular dystrophy (ABCA4), ornithine transcarbamylase (OTC) deficiency, Usher syndrome, alpha-1 antitrypsin deficiency, progressive familial intrahepatic cholestasis (PFIC) type I (ATP8B1 deficiency), type II (ABCB11), type III (ABCB4), or type IV (TJP2) and Cathepsin A deficiency.

According to another aspect, the disclosure provides a linear, single-stranded DNA (ssDNA) molecule comprising at least one nucleic acid sequence of interest flanked by at least one stem-loop structure at the 3′ end produced by the method of any one of the aspects and embodiments herein.

According to another aspect, the disclosure provides a lipid nanoparticle comprising the ssDNA molecule of any of the aspects and embodiments herein, and a lipid.

According to another aspect, the disclosure provides a pharmaceutical composition comprising the ssDNA molecule of any of the aspects or embodiments herein, or the lipid nanoparticle composition of any of the aspects and embodiments herein, and a pharmaceutically acceptable excipient.

According to another aspect, the disclosure provides a host cell comprising the ssDNA molecule of any of the aspects or embodiments herein or the lipid nanoparticle of any of the aspects or embodiments herein.

According to another aspect, the disclosure provides a method of treating a genetic disorder in a subject comprising administering a therapeutically effective amount of the ssDNA molecule of any of the aspects or embodiments herein, the lipid nanoparticle of any of the aspects or embodiments herein, or the pharmaceutical composition of any of the aspects or embodiments herein to the subject.

According to another aspect, the disclosure provides a method of delivering a therapeutic gene and/or a therapeutic protein to a subject comprising administering a therapeutically effective amount of the ssDNA molecule of any of the aspects or embodiments herein, the lipid nanoparticle of any of the aspects or embodiments herein, or the pharmaceutical composition of any of the aspects or embodiments herein to the subject.

According to another aspect, the disclosure provides a method of delivering a therapeutic gene and/or a therapeutic protein to a cell comprising contacting the cell with the ssDNA molecule of any of the aspects or embodiments herein, the lipid nanoparticle of any of the aspects or embodiments herein, or the pharmaceutical composition of any of the aspects or embodiments herein, thereby delivering the therapeutic gene and/or therapeutic protein to the cell.

According to another aspect, the disclosure provides a method of delivering a therapeutic gene to the nucleus of a cell comprising contacting the cell with the ssDNA molecule of any of the aspects or embodiments herein, the lipid nanoparticle of any of the aspects or embodiments herein, or the pharmaceutical composition of any of the aspects or embodiments herein, thereby delivering the therapeutic gene and/or therapeutic protein to the nucleus of the cell.

According to another aspect, the disclosure provides a method of minimizing an immune response in a subject, wherein the subject is being treated with a therapeutic gene or therapeutic protein, comprising administering a therapeutically effective amount of the ssDNA molecule of any of the aspects or embodiments herein, the lipid nanoparticle of any of the aspects or embodiments herein, or the pharmaceutical composition of any of the aspects or embodiments herein to the subject, wherein the nucleic acid of interest encodes the therapeutic gene or therapeutic protein.

### BRIEF DESCRIPTION OF DRAWINGS

Embodiments of the present disclosure, briefly summarized above and discussed in greater detail below, can be understood by reference to the illustrative embodiments of the disclosure depicted in the appended drawings. However, the appended drawings illustrate only typical embodiments of the disclosure and are therefore not to be considered limiting of scope, for the disclosure may admit to other equally effective embodiments.

FIG. 1 depicts schematic drawings of symmetric versus asymmetric inverted terminal repeat (ITR) oligos.

FIG. 2 shows synthesis of single-stranded DNA (ssDNA, SSD) via rolling circle amplification and enzymatic synthesis. Plasmid template (lane 2 and 7) was subject to rolling circle amplification to produce intermediate dsDNA molecule “A” (lane 3). This intermediate molecule was subject to enzymatic synthesis to produce closed-ended DNA (ceDNA) molecule “C” (lane 4 and 9). ceDNA was further processed using either Nb.BbvCI (lane 5) or Endonuclease V (lane 10) to produce ssDNA “I”. oc, open circular plasmid; sc, supercoiled plasmid; A, amplification product; C, ceDNA; I, ssDNA.

FIG. 3A shows the design of Endonuclease V substrates for single-stranded DNA (ssDNA) synthesis. As shown in FIG. 3A, inosine positions -1, -2, -5 and -7 are numbered in reference to the 3′ end of the left ITR (see SEQ ID NO: 4). In the RBE region of the left ITR (SEQ ID NO:4), certain nucleotides (nt) are modified from the AAV2 ITR sequence to minimize the CpG sites. This was advantageous to the present invention because CpG sites are known to activate the innate immune response, and methylation of CpG motifs can affect promoter function via, e.g., promoter silencing.

FIG. 3B depicts an exemplary ssDNA molecule containing hairpin ITRs with potential positions for inosine substitution, and with phosphorothioate (PS) bonds.

FIG. 4A shows schematics of the predicted secondary structures of inosine-modified left ITRs. Inosine position affects second-strand synthesis of ssDNA. The structure on the far left (i) is the standard (non-modified) structure. The structures designated (ii)-(v) model inosine modification of the left ITR, relative to the 3′ end. Red, green, and blue indicate a high, mid, or low probability of base pairing.

FIG. 4B shows schematics of the predicted secondary structures of inosine-modified left ITRs after Endonuclease V-mediated ssDNA synthesis. The structures designated (i)-(iv) show the predicted secondary structure of the left ITR, with inosine modification relative to the 3′ end. Red, green, and blue indicate a high, mid, or low probability of base pairing. The 3′ and 5′ end of each ITR is labeled.

FIG. 5 shows the results of Klenow fill-in of inosine containing single-stranded DNA, demonstrating that ssDNA conversion was successful. ceDNA containing no inosine or inosine at various positions within the left ITR (SEQ ID NO:) were generated via RAMP (lanes 2, 5, 8, 11, 14). ceDNA were subjected to Endonuclease V-mediated ssDNA synthesis (lanes 3, 7, 10, 15). The resulting products were treated with DNA polymerase I large (Klenow) fragment exo-(which lacks 3′->5′ and 5′->exonuclease activity) to facilitate second-strand synthesis (lanes 4, 7, 10, 13, 16). ceDNA and products of successful second-strand synthesis of ssDNA co-migrated.

FIG. 6 shows results demonstrating that the universal Endonuclease V-mediated synthesis protocol enables efficient ssDNA conversion across constructs. Multiple ceDNA with unique internal sequences were generated via RAMP. All ceDNA contained a left ITR with inosine present at the −1 position and a right ITR with an extended A-stem (SO-238; SEQ ID NO: 14) (lanes 3, 5, 7, 10, 11). ceDNA lacking inosine served as a control for Endonuclease V activity (lane 2). All ceDNA were subjected to Endonuclease V-mediated ssDNA synthesis (lanes 2, 4, 6, 8, 9, 12).

FIG. 7 illustrates an exemplary approach for the synthesis process by functional parts to enable a minimalist, universal synthesis approach. The conventional approach is GOI directed and thus unique to GOI, and requires enzyme/sequence optimization for GOI. The new process as described herein is left ITR directed, and is a universal approach, and uses modification-specific enzyme, e.g., Endonuclease V which is a DNA damage repair protein that recognizes and nicks inosine-containing DNA.

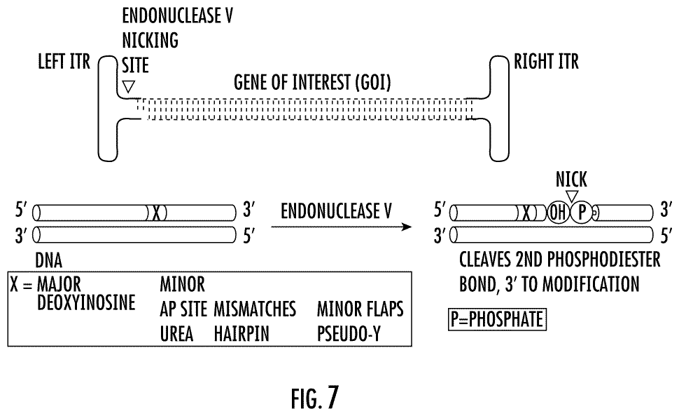

FIG. 8 illustrates the process of eliminating various ITR regions to arrive at the minimally required ssDNA as described herein.

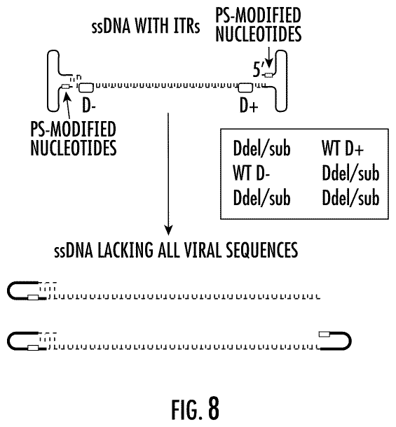

FIG. 9. Illustrates ssDNA variants to improve metabolic stability and promote higher gene expression.

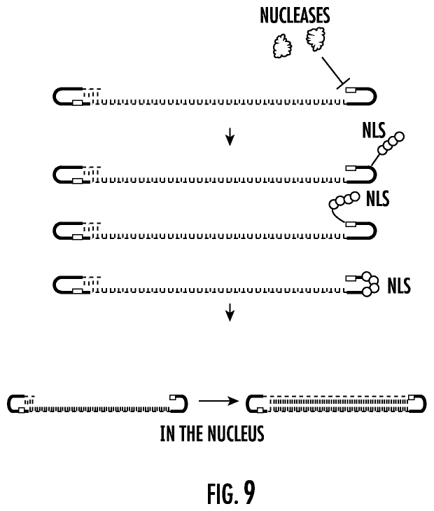

FIG. 10 illustrates exemplary modifications that inhibit nucleases and/or increase duplex stability in ITR configurations.

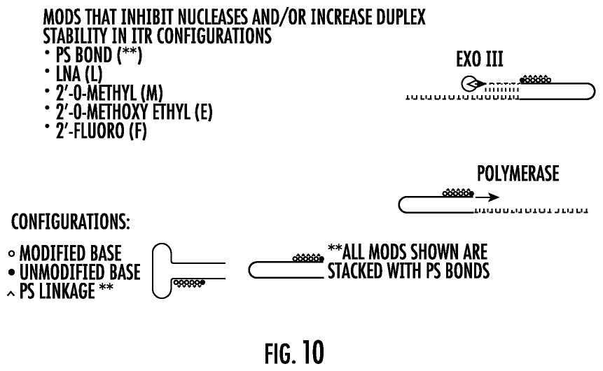

FIG. 11 depicts an exemplary LNP encapsulating ssDNA as described herein.

FIG. 12 illustrates ssDNA synthesis in the absence of phosphorothioate (PS) bonds to terminate T7 exonuclease using a ceDNA precursor with AAV-derived ITRs. On the left and middle are schematics of ceDNA precursors showing conversion to ssDNA with and without PS bonds, respectively. On the right is a gel showing efficient conversion of ceDNA to ssDNA after treatment of ceDNA with nicking enzyme and T7 exonuclease, with and without PS bonds in the ceDNA precursor.

FIG. 13 illustrates ssDNA synthesis in the absence of phosphorothioate (PS) bonds to terminate T7 exonuclease using a ceDNA precursor with simple hairpin closed ends. On the left and middle are schematics of ceDNA precursors with simple hairpin closed ends showing conversion to ssDNA with and without PS bonds, respectively. On the right is a gel showing efficient conversion of ceDNA to ssDNA after treatment of ceDNA with nicking enzyme and T7 exonuclease, with and without PS bonds in the ceDNA precursor.

FIGS. 14A-14D show schematics of ssDNA produced by treatment of ceDNA precursors with and without PS bonds, with AAV-derived or simple hairpin ends. Triangles indicate the location of the nick site. Arrows indicate the location of the priming site for Sanger run-off sequencing. Stars indicate the location of PS bonds. FIG. 14A: AAV-derived ITR end (right side), with PS bonds. FIG. 14B: AAV-derived ITR end (right side), no PS bonds. FIG. 14C: simple hairpin end (right side), with PS bonds. FIG. 14D: simple hairpin end (right side), no PS bonds. Dotted line on the right side in FIG. 14B and FIG. 14D indicate heterogeneity of endpoint sequence.

FIG. 15 illustrates an example of an end structure oligonucleotide and the strategy used to test sequence and structural requirements for T7 exonuclease termination. At the top is an example of an oligonucleotide sequence and predicted dsDNA structure, including a CH4-1 aptamer on the right side. At the bottom are schematics of predicted fragments produced by RsaI and EcoRI digestion, depending on whether T7 exonuclease is terminated by the structured region.

FIG. 16A shows the sequence (bottom) and schematic (top) of a full hilt oligonucleotide, which also includes a CH4-1 aptamer on the right side.

FIG. 16B shows the sequence (bottom) and schematic (top) of a half hilt oligonucleotide, which also includes a CH4-1 aptamer on the right side.

FIG. 16C shows the sequence (bottom) and schematic (top) of an extended half hilt oligonucleotide, which also includes a CH4-1 aptamer on the right side.

FIG. 16D shows the sequence (bottom) and schematic (top) of a bubble_v1 oligonucleotide, which also includes a CH4-1 aptamer on the right side.

FIG. 16E shows the sequence (bottom) and schematic (top) of a bubble_v19 oligonucleotide, which also includes a CH4-1 aptamer on the right side.

FIG. 16F shows the sequence (bottom) and schematic (top) of a loop oligonucleotide, which also includes a CH4-1 aptamer on the right side.

FIG. 16G shows the sequence (bottom) and schematic (top) of an oligonucleotide with PS bonds (“1-5” indicates that the oligonucleotide comprises 1, 2, 3, 4, or 5 PS bonds), which also includes a CH4-1 aptamer on the right side.

FIG. 16H shows the sequence (bottom) and schematic (top) of a control (no TS) oligonucleotide, which also includes a CH4-1 aptamer on the right side.

FIG. 17 shows gel analysis of restriction enzyme digest profiles of bubble_v1, bubble_v19, full hilt, half hilt, and extended half hilt oligonucleotides.

FIG. 18 shows gel analysis of restriction enzyme digest profiles of oligonucleotides with 5 PS bonds, 4 PS bonds, 3 bonds, 2 PS bonds, 1 PS bonds, or a loop oligonucleotide.

FIG. 19 illustrates schematic strategies for production of ssDNA using a full hilt structured motifs to terminate T7 exonuclease. Both sides illustrate the use of a full hilt structure. Additionally, the right side illustrates the inclusion of an aptamer encoded as double-stranded DNA, which only folds into a functional aptamer structure after the ssDNA is produced.

FIG. 20 illustrates schematic strategies for production of ssDNA using different structured motifs to terminate T7 exonuclease. Both sides illustrate the use of a half hilt structure. Additionally, the right side illustrates the inclusion of an aptamer encoded as double-stranded DNA, which only folds into a functional aptamer structure after the ssDNA is produced.

FIG. 21 illustrates a schematic strategy for production of ssDNA using Exonuclease III (Exo III) to degrade a nicked strand in the 3′→5′ direction. Termination of Exo III is controlled by the specific location of PS bonds (represented by circles connected by bent lines).

FIG. 22 shows gel analysis of ssDNA produced using Exo III, as compared to T7 exonuclease. The left shows the results of a two-step method. The right shows the results of a “one pot” method.

### DETAILED DESCRIPTION

### I. Definitions

Unless otherwise defined herein, scientific and technical terms used in connection with the present application shall have the meanings that are commonly understood by those of ordinary skill in the art to which this disclosure belongs. It should be understood that this disclosure is not limited to the particular methodology, protocols, and reagents, etc., described herein and as such can vary. The terminology used herein is for the purpose of describing particular embodiments only, and is not intended to limit the scope of the present disclosure, which is defined solely by the claims. Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), Fields Virology, 6th Edition, published by Lippincott Williams & Wilkins, Philadelphia, PA, USA (2013), Knipe, D. M. and Howley, P. M. (ed.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

As used herein, the term “AAV” or “adeno-associated virus” refer to single-stranded DNA parvoviruses that replicate only in cells. Certain functions of AAV are provided only by co-infecting a helper virus. Thirteen serotypes of AAV have been identified. General information and review of AAV can be found, e.g., in Carter, 1989, Handbook of Parvoviruses, Vol. 1, p. 169-228, and Berns, 1990, Virology, pp. 1743-1764, Raven Press, (New York).

As used herein, the phrase “anti-therapeutic nucleic acid immune response,” “immune response against a therapeutic nucleic acid,” “immune response against a transfer vector,” or the like refers to any immune response against a therapeutic nucleic acid, viral or non-viral in its origin. For example, in some embodiments, the immune response is specific to the transfer vector which can be single-stranded DNA, double-stranded DNA, single-stranded RNA, or double-stranded RNA. In other embodiments, the immune response is specific to single-stranded DNA, e.g., single-stranded synthetic DNA.

As used herein, the term “aptamer” refers to a nucleic acid molecule that is capable of binding to a particular molecule of interest with high affinity and specificity (Tuerk and Gold, Science 249:505 (1990); Ellington and Szostak, Nature 346:818 (1990)). For example, aptamers may be composed of DNA or RNA, or may comprise non-natural nucleotides and nucleotide analogs (e.g., locked DNA or peptide nucleic acids [PNAs]) that have high affinity to a protein localized in the nucleus or the membrane thereof.

As used herein, the terms “cell-free,” “cell-free synthesis,” “cell-free production,” “synthetic closed-ended DNA vector production” and “synthetic production” and all other related counterparts are used interchangeably and refer to the production of one or more molecules in a manner that does not involve replication or other multiplication of the molecule by or inside of a cell or using a cellular extract. Synthetic production avoids contamination of the produced molecule with cellular contaminants (e.g., cellular proteins or cellular nucleic acids) and further avoids unwanted cellular-specific modification of the molecule during the production process (e.g., methylation or glycosylation or other post-translational modification).

As used herein, the terms “single-stranded DNA molecule”, “ssDNA molecule”, or “SSD molecule” refer to a deoxyribonucleic acid (DNA) molecule comprising at least one single-stranded nucleic acid sequence flanked by at least one stem-loop structure at the 3′ end. In some embodiments, the single-stranded DNA molecule further comprises at least one stem-loop structure at the 5′ end. As used herein, a single-stranded DNA molecule may comprise regions of double-stranded DNA (or partial duplexes), e.g., a stem-loop structure, e.g., an inverted terminal repeat or portion thereof, at the terminal end(s), e.g., the 3′ end and/or the 5′ end. In some embodiments, a ssDNA molecule is a synthetic ssDNA molecule. In some embodiments, a ssDNA molecule comprises at least one stem-loop structure at the 5′ end and at least one stem-loop structure at the 3′ end.

As used herein, the term “single-stranded (ss) synthetic DNA molecules”, “single-stranded (ss) synthetic vectors”, “synthetic production of ss DNA molecules” and “synthetic production of ss vectors” refers to a single-stranded (ss) synthetic DNA molecule (ssDNA), a single-stranded vector and synthetic production methods thereof in an entirely cell-free environment. The production may involve one or more molecules in a manner that does not involve replication or other multiplication of the molecule by or inside of a cell or using a cellular extract. Synthetic production avoids contamination of the produced molecule with cellular contaminants, e.g., cellular proteins or cellular nucleic acid, viral protein or DNA, insect protein or DNA and further minimizes unwanted cellular-specific modification of the molecule during the production process, e.g., methylation or glycosylation or other post-translational modification.

As used herein, the term “gap” refers to a discontinued portion of synthetic DNA vector of the present disclosure, creating a stretch of single-stranded DNA portion in otherwise double-stranded DNA. The gap can be 1 nucleotide to 100 nucleotides long in length. Typical gaps, designed and created by the methods described herein and synthetic vectors generated by the methods can be, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 nucleotides (nt) long in length. Exemplified gaps in the present disclosure can be 1 nt to 10 nt long, 1 to 20 nt long, 1 to 30 nt long, or any length. According to some embodiments, gaps can be present 5′ upstream of an expression cassette. According to some embodiments, gaps can be present 3′ downstream of an expression cassette. According to some embodiments, gaps can be present both 5′ upstream and 3′ downstream of an expression cassette.

As used herein, the term “nick” refers to a discontinuity in a double-stranded DNA molecule where there is no phosphodiester bond between adjacent nucleotides of one strand typically through damage or enzyme action. It is to be understood that one or more nicks allow for the release of torsion in the DNA strand during replication and that nicks play a role in facilitating binding of transcriptional machinery. According to some embodiments, a single-stranded break (“nick”) in DNA can be formed by the hydrolysis and subsequent removal of a phosphate group within the helical backbone.

As used herein, the term “ceDNA” refers to capsid-free closed-ended linear double-stranded (ds) duplex DNA for non-viral gene transfer, synthetic or otherwise. Detailed description of ceDNA is described in International Patent Application No. PCT/US2017/020828, filed Mar. 3, 2017 (published as International patent publication No. WO2017152149A1), the entire contents of which are expressly incorporated herein by reference. Certain methods for the production of ceDNA comprising various inverted terminal repeat (ITR) sequences and configurations using cell-based methods are described in Example 1 of International applications PCT/US18/49996, filed Sep. 7, 2018 (published as International patent publication No. WO 2019/051255 A1), and PCT/US2018/064242, filed Dec. 6, 2018 (published as International patent publication No. WO 2019/113310 A1), each of which is incorporated herein in its entirety by reference. Certain methods for the production of synthetic ceDNA vectors comprising various ITR sequences and configurations are described, e.g., in International application PCT/US2019/14122, filed Jan. 18, 2019 (published as International patent publication No. WO 2019/143885 A1), the entire content of which is incorporated herein by reference. As used herein, the terms “ceDNA vector” and “ceDNA” are used interchangeably. According to some embodiments, the ceDNA is a closed-ended linear duplex (CELiD) CELID DNA. According to some embodiments, the ceDNA is a DNA-based minicircle. According to some embodiments, the ceDNA is a minimalistic immunological-defined gene expression (MIDGE)-vector. According to some embodiments, the ceDNA is a ministring DNA. According to some embodiments, the ceDNA is a Doggybone™ DNA. According to some embodiments, the ceDNA comprises one or more phosphorothioate-modified nucleotides. According to some embodiments, the ceDNA comprises no phosphorothioate-modified nucleotides.

As used herein, the term “neDNA” or “nicked ceDNA” refers to a closed-ended DNA having a nick or a gap of 1-100 base pairs a stem region or spacer region upstream of an open reading frame (e.g., a promoter and transgene to be expressed).

As used herein, the terms “inverted terminal repeat” or “ITR” refer to a nucleic acid sequence located at the 5′ and/or 3′ terminus of the ssDNA molecules disclosed herein, which comprises at least one stem-loop structure comprising a partial duplex and at least one loop.

As used herein, the term “stem-loop structure” refers to a nucleic acid structure comprising at least one double-stranded region (referred to herein as a “stem”) and at least one single-stranded region (referred to herein as a “loop”). In some embodiments, a stem-lop structure is a hairpin structure. In some embodiments, a stem-loop structure comprises more than one stem and more than one loop. In some embodiments, a loop is located at the end of a stem (such that a single loop connects the two strands of a duplex stem, e.g., as in a hairpin structure). In some embodiments, a loop may be located between two stems (which may be referred to herein as a “bulge” or a “bubble”), such that the loop connects two strands of different stems. In some embodiments, as described in more detail herein, a stem-loop structure may comprise more complex secondary structures comprising multiple stems and multiple loops.

According to some embodiments, the 5′ and/or 3′ terminus of the ssDNA molecules disclosed herein comprise inverted terminal repeats (ITRs) of about 145 nucleotides at both ends, or fragments thereof. The terminal 125 nucleotides in each ITR form a palindromic double-stranded T-shaped hairpin structure, in which the A-A′ palindrome forms the stem, and the two smaller palindromes, B-B′ and the C-C′, form the cross-arms of the T. The other 20 nucleotides in ITR remain single-stranded, and are called the D sequence. The D(−) sequence (also referred to herein as “the ssD(−) sequence”) is at the 3′ end, and the complementary D(+) sequence (also referred to herein as “the ssD(+) sequence”) is at the 5′ end. Second-strand DNA synthesis turns both ssD(−) and ssD(+) sequences into a double-stranded (ds) D(+) sequence, each of which comprises a D region and a D′ region. Ling et al. J Virol. 2015 Jan. 15; 89 (2): 952-61, WO2016081927A2, incorporated by reference in its entirety herein, described ssD(+)-sequence-substituted ssAAV genomes. ssD(−) and ssD(+) have been reported to contain one or more transcription factor binding sites and to be required for packaging and replication (Ling et al. J Virol. 2015 Jan. 15; 89(2):952-61; WO2016081927A2, incorporated by reference in its entirety herein).

According to some embodiments, the ITR may be a viral ITR (e.g., AAV or other dependovirus), a sequence derived or modified from a viral ITR (e.g., truncation, deletion, substitution, insertion and/or addition), or an entirely artificial sequence (e.g., the ITRs contain no sequences derived from a virus). The ITR may further comprise one stem-loop structure (e.g., a “hairpin”), or more than one stem-loop structure. For example, the ITR may comprise two stem-loop structures (e.g., a “hammerhead”, “doggy-bone”, or “dumbbell”), three stem-loop structures (e.g., “cruciform”), or more complex structures (e.g., quadruplex stem-loop structure). The ITR may comprise an aptamer sequence or one or more chemical modifications. The ITR can be made entirely out of an aptamer sequence having at least one stem region and at least one loop region.

According to some embodiments, the “ITR” can be artificially synthesized using a set of oligonucleotides comprising one or more desirable functional sequences (e.g., palindromic sequence). The ITR sequence can be an artificial AAV ITR, an artificial non-AAV ITR, or an ITR physically derived from a viral AAV ITR (e.g., ITR fragments removed from a viral genome). For example, the ITR can be derived from the family Parvoviridae, which encompasses parvoviruses and dependoviruses (e.g., canine parvovirus, bovine parvovirus, mouse parvovirus, porcine parvovirus, human parvovirus B-19), or the SV40 hairpin that serves as the origin of SV40 replication can be used as an ITR, which can further be modified by truncation, substitution, deletion, insertion and/or addition. Parvoviridae family viruses consist of two subfamilies: Parvovirinae, which infect vertebrates, and Densovirinae, which infect invertebrates. Dependoparvoviruses include the viral family of the adeno-associated viruses (AAV) which are capable of replication in vertebrate hosts including, but not limited to, human, primate, bovine, canine, equine and ovine species. Typically, ITR sequences can be derived not only from AAV, but also from Parvovirus, lentivirus, goose virus, B19, in the configurations of wildtype, “doggy bone” and “dumbbell shape”, symmetrical or even asymmetrical ITR orientation. Although the ITRs are typically present in both 5′ and 3′ ends of an AAV vector, in a single-stranded DNA (ssDNA) molecule the ITR can be present in only one of end of the linear vector. For example, the ITR can be present on the 5′ end only. Some other cases, the ITR can be present on the 3′ end only in a single-stranded DNA (ssDNA) molecule. For convenience herein, an ITR located 5′ to (“upstream of”) an expression cassette in a single-stranded DNA (ssDNA) molecule is referred to as a “5′ ITR”, and an ITR located 3′ to (“downstream of”) an expression cassette in a single-stranded DNA (ssDNA) molecule is referred to as a “3′ ITR”.

As used herein, a “wild-type ITR” or “WT-ITR” refers to the sequence of a naturally occurring ITR sequence in an AAV genome or other dependovirus that remains, e.g., Rep binding activity and Rep nicking ability. The nucleotide sequence of a WT-ITR from any AAV serotype may slightly vary from the canonical naturally occurring sequence due to degeneracy of the genetic code or drift, and therefore WT-ITR sequences encompasses for use herein include WT-ITR sequences as result of naturally occurring changes (e.g., a replication error).

As used herein, the term “substantially symmetrical WT-ITRs” or a “substantially symmetrical WT-ITR pair” refers to a pair of WT-ITRs within a single-stranded DNA (ssDNA) molecule that are both wild type ITRs that have an inverse complement sequence across their entire length. For example, an ITR can be considered to be a wild-type sequence, even if it has one or more nucleotides that deviate from the canonical naturally occurring canonical sequence, so long as the changes do not affect the physical and functional properties and overall three-dimensional structure of the sequence (secondary and tertiary structures). In some aspects, the deviating nucleotides represent conservative sequence changes. As one non-limiting example, a sequence that has at least 95%, 96%, 97%, 98%, or 99% sequence identity to the canonical sequence (as measured, e.g., using BLAST at default settings), and also has a symmetrical three-dimensional spatial organization to the other WT-ITR such that their 3D structures are the same shape in geometrical space. The substantially symmetrical WT-ITR has the same ssD(−)/ssD(+), A-A′, C-C′ and B-B′ loops in 3D space. A substantially symmetrical WT-ITR can be functionally confirmed as WT by determining that it has an operable Rep binding site (RBE or RBE′) and terminal resolution site (TRS) that pairs with the appropriate Rep protein. One can optionally test other functions, including transgene expression under permissive conditions.

As used herein, the phrases of “modified ITR” or “mod-ITR” or “mutant ITR” are used interchangeably and refer to an ITR with a mutation in at least one or more nucleotides as compared to the WT-ITR from the same serotype. The mutation can result in a change in one or more of ssD(−) or ssD(+), A, A′, C, C′, B, B′ regions in the ITR, and can result in a change in the three-dimensional spatial organization (i.e., its 3D structure in geometric space) as compared to the 3D spatial organization of a WT-ITR of the same serotype.

As used herein, the term “asymmetric ITRs” also referred to as “asymmetric ITR pairs” refers to a pair of ITRs within a single ssDNA vector that are not inverse complements across their full length. As one non-limiting example, an asymmetric ITR pair does not have a symmetrical three-dimensional spatial organization to their cognate ITR such that their 3D structures are different shapes in geometrical space. Stated differently, an asymmetrical ITR pair have the different overall geometric structure, i.e., they have different organization of their ssD(−)/ssD(+), A, A′, C, C′, B, and B′ regions in 3D space (e.g., one ITR may have no ssD(−) and a short C-C′ arm and/or short B-B′ arm and other ITR may have no ssD(+), but a normal AAV C-C′ arm and truncated B-B′ arm as compared to the cognate ITR). The difference in sequence between the two ITRs may be due to one or more nucleotide addition, deletion, truncation, or point mutation. In one embodiment, one ITR of the asymmetric ITR pair may be a wild-type AAV ITR sequence and the other ITR a modified ITR as defined herein (e.g., a non-wild-type or synthetic ITR sequence). In another embodiment, neither ITRs of the asymmetric ITR pair is a wild-type AAV sequence and the two ITRs are modified ITRs that have different shapes in geometrical space (i.e., a different overall geometric structure). In some embodiments, one mod-ITRs of an asymmetric ITR pair can have a short C-C′ arm and the other ITR can have a different modification (e.g., a single arm, or a short B-B′ arm etc.) such that they have different three-dimensional spatial organization as compared to the cognate asymmetric mod-ITR.

As used herein, the term “symmetric ITRs” refers to a pair of ITRs within an ssDNA vector that are mutated or modified relative to wild-type dependoviral ITR sequences and are inverse complements across their full length. Neither ITRs are wild type ITR AAV2 sequences (i.e., they are a modified ITR, also referred to as a mutant ITR), and can have a difference in sequence from the wild type ITR due to nucleotide addition, deletion, substitution, truncation, or point mutation. For convenience herein, an ITR located 5′ to (upstream of) an expression cassette in a single-stranded DNA (ssDNA) molecule is referred to as a “5′ ITR” and an ITR located 3′ to (downstream of) an expression cassette in a single-stranded DNA (ssDNA) molecule is referred to as a “3′ ITR”.

As used herein, the terms “substantially symmetrical modified-ITRs” or a “substantially symmetrical mod-ITR pair” refers to a pair of modified-ITRs within a single-stranded DNA (ssDNA) molecule (e.g., synthetic vector, e.g., single-stranded (ss) synthetic vector) that have an inverse complement sequence across their entire length. For example, the modified ITR can be considered substantially symmetrical, even if it has some nucleotide sequences that deviate from the inverse complement sequence so long as the changes do not affect the properties and overall shape. As one non-limiting example, a sequence that has at least 85%, 90%, 95%, 96%, 97%, 98%, or 99% sequence identity to the canonical sequence (as measured using BLAST at default settings), and also has a symmetrical three-dimensional spatial organization to their cognate modified ITR such that their 3D structures are the same shape in geometrical space. Stated differently, a substantially symmetrical modified-ITR pair have the same stem-loop structures organized in 3D space. In some embodiments, the ITRs from a mod-ITR pair may have different reverse complement nucleotide sequences but still have the same symmetrical three-dimensional spatial organization—that is both ITRs have mutations that result in the same overall 3D shape. For example, in a viral-derived ITR, one ITR (e.g., 5′ ITR) in a mod-ITR pair can be from one serotype, and the other ITR (e.g., 3′ ITR) can be from a different serotype, however, both can have the same corresponding mutation (e.g., if the 5′ ITR has a deletion in the C region, the cognate modified 3′ ITR from a different serotype has a deletion at the corresponding position in the C′ region), such that the modified ITR pair has the same symmetrical three-dimensional spatial organization. In such embodiments, each ITR in a modified ITR pair can be from different serotypes (e.g., AAV1, 2, 3, 4, 5, 6, 7, 8, 9, 10, 11, and 12) such as the combination of AAV2 and AAV6, with the modification in one ITR reflected in the corresponding position in the cognate ITR from a different serotype. In one embodiment, a substantially symmetrical modified ITR pair refers to a pair of modified ITRs (mod-ITRs) so long as the difference in nucleotide sequences between the ITRs does not affect the properties or overall shape and they have substantially the same shape in 3D space. As a non-limiting example, a mod-ITR that has at least 95%, 96%, 97%, 98% or 99% sequence identity to the canonical mod-ITR as determined by standard means well known in the art such as BLAST (Basic Local Alignment Search Tool), or BLASTN at default settings, and also has a symmetrical three-dimensional spatial organization such that their 3D structure is the same shape in geometric space. A substantially symmetrical mod-ITR pair has the same ssD(−)/ssD(+), A, A′, C, C′, B and B′ regions in 3D space, e.g., if a modified ITR in a substantially symmetrical mod-ITR pair has a deletion of a C-C′ arm, then the cognate mod-ITR has the corresponding deletion of the C-C′ loop and also has a similar 3D structure of the remaining A and B-B′ loops in the same shape in geometric space of its cognate mod-ITR.

As used herein, the term “flanking” refers to a relative position of one nucleic acid sequence with respect to another nucleic acid sequence. Generally, in the sequence ABC, B is flanked by A and C. The same is true for the arrangement AxBxC. Thus, a flanking sequence precedes or follows a flanked sequence but need not be contiguous with, or immediately adjacent to the flanked sequence. In one embodiment, the term flanking refers to terminal repeats at each end of the linear single-stranded DNA (ssDNA) molecule.

As defined herein, “reporter” or “reporters” refer to a protein or proteins that can be used to provide detectable read-outs. Reporters generally produce a measurable signal such as fluorescence, color, or luminescence. Reporter protein coding sequences encode proteins whose presence in the cell or organism is readily observed. For example, fluorescent proteins cause a cell to fluoresce when excited with light of a particular wavelength, luciferases cause a cell to catalyze a reaction that produces light, and enzymes such as β-galactosidase convert a substrate to a colored product. Exemplary reporter polypeptides useful for experimental or diagnostic purposes include, but are not limited to β-lactamase, β-galactosidase (LacZ), alkaline phosphatase (AP), thymidine kinase (TK), green fluorescent protein (GFP) and other fluorescent proteins, chloramphenicol acetyltransferase (CAT), luciferase, and others well known in the art.

As used herein, the term “effector protein” refers to a polypeptide that provides a detectable read-out, either as, for example, a reporter polypeptide, or more appropriately, as a polypeptide that kills a cell, e.g., a toxin, or an agent that renders a cell susceptible to killing with a chosen agent or lack thereof. Effector proteins include any protein or peptide that directly targets or damages the host cell's DNA and/or RNA. For example, effector proteins can include, but are not limited to, a restriction endonuclease that targets a host cell DNA sequence (whether genomic or on an extrachromosomal element), a protease that degrades a polypeptide target necessary for cell survival, a DNA gyrase inhibitor, and a ribonuclease-type toxin. In some embodiments, the expression of an effector protein controlled by a synthetic biological circuit as described herein can participate as a factor in another synthetic biological circuit to thereby expand the range and complexity of a biological circuit system's responsiveness.

Transcriptional regulators refer to transcriptional activators and repressors that either activate or repress transcription of a gene of interest. Promoters are regions of nucleic acid that initiate transcription of a particular gene. Transcriptional activators typically bind nearby to transcriptional promoters and recruit RNA polymerase to directly initiate transcription. Repressors bind to transcriptional promoters and sterically hinder transcriptional initiation by RNA polymerase. Other transcriptional regulators may serve as either an activator or a repressor depending on where they bind and cellular and environmental conditions. Non-limiting examples of transcriptional regulator classes include, but are not limited to homeodomain proteins, zinc-finger proteins, winged-helix (forkhead) proteins, and leucine-zipper proteins.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutically active substances is well known in the art. Supplementary active ingredients can also be incorporated into the compositions. The phrase “pharmaceutically-acceptable” refers to molecular entities and compositions that do not produce a toxic, an allergic, or similar untoward reaction when administered to a host.

As used herein, the term “in vivo” refers to assays or processes that occur in or within an organism, such as a multicellular animal. In some of the aspects described herein, a method or use can be said to occur “in vivo” when a unicellular organism, such as a bacterium, is used. The term “ex vivo” refers to methods and uses that are performed using a living cell with an intact membrane that is outside of the body of a multicellular animal or plant, e.g., explants, cultured cells, including primary cells and cell lines, transformed cell lines, and extracted tissue or cells, including blood cells, among others. The term “in vitro” refers to assays and methods that do not require the presence of a cell with an intact membrane, such as cellular extracts, and can refer to the introducing of a programmable synthetic biological circuit in a non-cellular system, such as a medium not comprising cells or cellular systems, such as cellular extracts.

As used herein, the term “promoter” refers to any nucleic acid sequence that regulates the expression of another nucleic acid sequence by driving transcription of the nucleic acid sequence, which can be a heterologous target gene encoding a protein or an RNA. Promoters can be constitutive, inducible, repressible, tissue-specific, or any combination thereof. A promoter is a control region of a nucleic acid sequence at which initiation and rate of transcription of the remainder of a nucleic acid sequence are controlled. A promoter can also contain genetic elements at which regulatory proteins and molecules can bind, such as RNA polymerase and other transcription factors. Within the promoter sequence will be found a transcription initiation site, as well as protein binding domains responsible for the binding of RNA polymerase. Eukaryotic promoters will often, but not always, contain “TATA” boxes and “CAT” boxes. Various promoters, including inducible promoters, may be used to drive the expression of transgenes in the single-stranded (ssDNA) molecules disclosed herein. A promoter sequence may be bounded at its 3′ terminus by the transcription initiation site and extends upstream (5′ direction) to include the minimum number of bases or elements necessary to initiate transcription at levels detectable above background.

As used herein, the terms “expression cassette” and “expression unit” are used interchangeably and refer to a heterologous DNA sequence that is operably linked to a promoter or other DNA regulatory sequence sufficient to direct transcription of a transgene of a DNA vector, e.g., a single-stranded (ssDNA) molecule. Suitable promoters include, for example, tissue specific promoters. Promoters can also be of AAV origin.

As used herein, the term “regenerated”, when referring to a “regenerated double-stranded expression cassette” or a “regenerated double-stranded transgene”, refers to the double-stranded expression cassette or double-stranded transgene that is formed after a ssDNA molecule has been transported to the nucleus of a host cell and is responsive to DNA polymerase activity that creates double-stranded DNA from the ssDNA by filling in the single strand portion of the ssDNA molecule.

As used herein, “operably linked” refers to a juxtaposition wherein the components so described are in a relationship permitting them to function in their intended manner. As an example, a promoter is operably linked to a coding sequence if the promoter affects its transcription or expression. A promoter can be said to drive expression or drive transcription of the nucleic acid sequence that it regulates. The phrases “operably linked,” “operatively positioned,” “operatively linked,” “under control,” and “under transcriptional control” indicate that a promoter is in a correct functional location and/or orientation in relation to a nucleic acid sequence it regulates to control transcriptional initiation and/or expression of that sequence. An “inverted promoter,” as used herein, refers to a promoter in which the nucleic acid sequence is in the reverse orientation, such that what was the coding strand is now the non-coding strand, and vice versa. Inverted promoter sequences can be used in various embodiments to regulate the state of a switch. In addition, in various embodiments, a promoter can be used in conjunction with an enhancer.

The terms “DNA regulatory sequences,” “control elements,” and “regulatory elements,” used interchangeably herein, refer to transcriptional and translational control sequences, such as promoters, enhancers, polyadenylation signals, terminators, protein degradation signals, and the like, that provide for and/or regulate transcription of a non-coding sequence (e.g., DNA-targeting RNA) or a coding sequence (e.g., site-directed modifying polypeptide, or Cas9/Csn1 polypeptide) and/or regulate translation of an encoded polypeptide.

The term “enhancer” as used herein refers to a cis-acting regulatory sequence (e.g., 50-1,500 base pairs) that binds one or more proteins (e.g., activator proteins, or transcription factor) to increase transcriptional activation of a nucleic acid sequence. Naturally, enhancers can be positioned up to 1,000,000 base pairs upstream of the gene start site or downstream of the gene start site that they regulate. An enhancer can be positioned within an intronic region, or in the exonic region of an unrelated gene. A cis-acting enhancer sequence of 20-200 base pairs can be typically used to increase expression of a transgene.

A promoter can be one naturally associated with a gene or sequence, as can be obtained by isolating the 5′ non-coding sequences located upstream of the coding segment and/or exon of a given gene or sequence. Such a promoter can be referred to as “endogenous.” Similarly, in some embodiments, an enhancer can be one naturally associated with a nucleic acid sequence, located either downstream or upstream of that sequence. In some embodiments, a coding nucleic acid segment is positioned under the control of a “recombinant promoter” or “heterologous promoter,” both of which refer to a promoter that is not normally associated with the encoded nucleic acid sequence that it is operably linked to in its natural environment. Similarly, a “recombinant or heterologous enhancer” refers to an enhancer not normally associated with a given nucleic acid sequence in its natural environment. Such promoters or enhancers can include promoters or enhancers of other genes; promoters or enhancers isolated from any other prokaryotic, viral, or eukaryotic cell; and synthetic promoters or enhancers that are not “naturally occurring,” i.e., comprise different elements of different transcriptional regulatory regions, and/or mutations that alter expression through methods of genetic engineering that are known in the art. In addition to producing nucleic acid sequences of promoters and enhancers synthetically, promoter sequences can be produced using recombinant cloning and/or nucleic acid amplification technology, including PCR, in connection with the synthetic biological circuits and modules disclosed herein (see, e.g., U.S. Pat. Nos. 4,683,202, 5,928,906, each incorporated herein by reference). Furthermore, it is contemplated that control sequences that direct transcription and/or expression of sequences within non-nuclear organelles such as mitochondria, chloroplasts, and the like, can be employed as well.

As described herein, an “inducible promoter” is one that is characterized by initiating or enhancing transcriptional activity when in the presence of, influenced by, or contacted by an inducer or inducing agent. An “inducer” or “inducing agent,” as defined herein, can be endogenous, or a normally exogenous compound or protein that is administered in such a way as to be active in inducing transcriptional activity from the inducible promoter. In some embodiments, the inducer or inducing agent, i.e., a chemical, a compound or a protein, can itself be the result of transcription or expression of a nucleic acid sequence (i.e., an inducer can be an inducer protein expressed by another component or module), which itself can be under the control or an inducible promoter. In some embodiments, an inducible promoter is induced in the absence of certain agents, such as a repressor. Examples of inducible promoters include but are not limited to, tetracycline, metallothionine, ecdysone, mammalian viruses (e.g., the adenovirus late promoter; and the mouse mammary tumor virus long terminal repeat (MMTV-LTR)) and other steroid-responsive promoters, rapamycin responsive promoters and the like.

The term “subject” as used herein refers to a human or animal, to whom treatment, including prophylactic treatment, with the single-stranded (ssDNA) molecule according to the present disclosure, is provided. Usually, the animal is a vertebrate such as, but not limited to, a primate, rodent, domestic animal or game animal. Primates include but are not limited to, chimpanzees, cynomolgus monkeys, spider monkeys, and macaques, e.g., Rhesus. Rodents include mice, rats, woodchucks, ferrets, rabbits and hamsters. Domestic and game animals include, but are not limited to, cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In certain embodiments of the aspects described herein, the subject is a mammal, e.g., a primate or a human. A subject can be male or female. Additionally, a subject can be an infant or a child. In some embodiments, the subject can be a neonate or an unborn subject, e.g., the subject is in utero. Preferably, the subject is a mammal. The mammal can be a human, non-human primate, mouse, rat, dog, cat, horse, or cow, but is not limited to these examples. Mammals other than humans can be advantageously used as subjects that represent animal models of diseases and disorders. In addition, the methods and compositions described herein can be used for domesticated animals and/or pets. A human subject can be of any age, gender, race or ethnic group, e.g., Caucasian (white), Asian, African, black, African American, African European, Hispanic, Mideastern, etc. In some embodiments, the subject can be a patient or other subject in a clinical setting. In some embodiments, the subject is already undergoing treatment. In some embodiments, the subject is an embryo, a fetus, neonate, infant, child, adolescent, or adult. In some embodiments, the subject is a human fetus, human neonate, human infant, human child, human adolescent, or human adult. In some embodiments, the subject is an animal embryo, or non-human embryo or non-human primate embryo. In some embodiments, the subject is a human embryo.

As used herein, the term “host cell” includes any cell type that is susceptible to transformation, transfection, transduction, and the like with a single-stranded (ssDNA) molecule described by the present disclosure. As non-limiting examples, a host cell can be an isolated primary cell, pluripotent stem cells, CD34⁺ cells, induced pluripotent stem cells, or any of a number of immortalized cell lines (e.g., HepG2 cells). Alternatively, a host cell can be an in situ or in vivo cell in a tissue, organ or organism. Furthermore, a host cell can be a target cell of, for example, a mammalian subject (e.g., human patient in need of gene therapy).

As used herein, the term “exogenous” refers to a substance present in a cell other than its native source. The term “exogenous” when used herein can refer to a nucleic acid (e.g., a nucleic acid encoding a polypeptide) or a polypeptide that has been introduced by a process involving the hand of man into a biological system such as a cell or organism in which it is not normally found and one wishes to introduce the nucleic acid or polypeptide into such a cell or organism. Alternatively, “exogenous” can refer to a nucleic acid or a polypeptide that has been introduced by a process involving the hand of man into a biological system such as a cell or organism in which it is found in relatively low amounts and one wishes to increase the amount of the nucleic acid or polypeptide in the cell or organism, e.g., to create ectopic expression or levels. In contrast, the term “endogenous” refers to a substance that is native to the biological system or cell.

The terms “polynucleotide” and “nucleic acid,” used interchangeably herein, refer to a polymeric form of nucleotides of any length, either ribonucleotides or deoxyribonucleotides. Thus, this term includes single, double, or multi-stranded DNA or RNA, genomic DNA, cDNA, DNA-RNA hybrids, or a polymer including purine and pyrimidine bases or other natural, chemically or biochemically modified, non-natural, or derivatized nucleotide bases. “Oligonucleotide” generally refers to polynucleotides of between about 5 and about 100 nucleotides of single- or double-stranded DNA. However, for the purposes of this disclosure, there is no upper limit to the length of an oligonucleotide. Oligonucleotides are also known as “oligomers” or “oligos” and may be isolated from genes, or chemically synthesized by methods known in the art. The terms “polynucleotide” and “nucleic acid” should be understood to include, as applicable to the embodiments being described, single-stranded (such as sense or antisense) and double-stranded polynucleotides. According to some embodiments, the nucleic acid is a single-stranded DNA (ssDNA) molecule described by the present disclosure. DNA may be in the form of, e.g., antisense molecules, plasmid DNA, DNA-DNA duplexes, pre-condensed DNA, PCR products, vectors (P1, PAC, BAC, YAC, artificial chromosomes), expression cassettes, chimeric sequences, chromosomal DNA, or derivatives and combinations of these groups. DNA may be in the form of minicircle, plasmid, bacmid, minigene, ministring DNA (linear covalently closed DNA vector), closed-ended linear duplex DNA (CELiD or ceDNA), doggybone (dbDNA™) DNA, dumbbell shaped DNA, minimalistic immunological-defined gene expression (MIDGE)-vector, viral vector or nonviral vectors. RNA may be in the form of small interfering RNA (siRNA), Dicer-substrate dsRNA, small hairpin RNA (shRNA), asymmetrical interfering RNA (aiRNA), microRNA (miRNA), mRNA, rRNA, tRNA, viral RNA (vRNA), and combinations thereof. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs and/or modified residues include, without limitation, phosphorothioates, phosphorodiamidate morpholino oligomer (morpholino), phosphoramidates, methyl phosphonates, chiral-methyl phosphonates, 2′-O-methyl ribonucleotides, locked nucleic acid (LNA™), and peptide nucleic acids (PNAs). Unless specifically limited, the term encompasses nucleic acids containing known analogues of natural nucleotides that have similar binding properties as the reference nucleic acid. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated.

An “inhibitory polynucleotide” as used herein refers to a DNA or RNA molecule that reduces or prevents expression (transcription or translation) of a second (target) polynucleotide. Inhibitory polynucleotides include antisense polynucleotides, ribozymes, and external guide sequences. The term “inhibitory polynucleotide” further includes DNA and RNA molecules, e.g., RNAi that encode the actual inhibitory species, such as DNA molecules that encode ribozymes.

“Nucleotides” contain a sugar deoxyribose (DNA) or ribose (RNA), a base, and a phosphate group. Nucleotides are linked together through the phosphate groups.

“Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides.

By “hybridizable” or “complementary” or “substantially complementary” it is meant that a nucleic acid (e.g., RNA) includes a sequence of nucleotides that enables it to non-covalently bind, i.e. form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. As is known in the art, standard Watson-Crick base-pairing includes: adenine (A) pairing with thymidine (T), adenine (A) pairing with uracil (U), and guanine (G) pairing with cytosine (C). In addition, it is also known in the art that for hybridization between two RNA molecules (e.g., dsRNA), guanine (G) base pairs with uracil (U). For example, G/U base-pairing is partially responsible for the degeneracy (i.e., redundancy) of the genetic code in the context of tRNA anti-codon base-pairing with codons in mRNA. In the context of this disclosure, a guanine (G) of a protein-binding segment (dsRNA duplex) of a subject DNA-targeting RNA molecule is considered complementary to a uracil (U), and vice versa. As such, when a G/U base-pair can be made at a given nucleotide position a protein-binding segment (dsRNA duplex) of a subject DNA-targeting RNA molecule, the position is not considered to be non-complementary, but is instead considered to be complementary.

The term “nucleic acid construct” as used herein refers to a nucleic acid molecule, either single- or double-stranded, which is isolated from a naturally occurring gene or which is modified to contain segments of nucleic acids in a manner that would not otherwise exist in nature or which is synthetic. The term nucleic acid construct is synonymous with the term “expression cassette” when the nucleic acid construct contains the control sequences required for expression of a coding sequence of the present disclosure. An “expression cassette” includes a DNA coding sequence operably linked to a promoter. The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

As used herein, the term “sequence identity” refers to the relatedness between two nucleotide sequences. For purposes of the present disclosure, the degree of sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows: (Identical Deoxyribonucleotides.times.100)/(Length of Alignment−Total Number of Gaps in Alignment). The length of the alignment is preferably at least 10 nucleotides, preferably at least 25 nucleotides more preferred at least 50 nucleotides and most preferred at least 100 nucleotides.

As used herein, the term “homology” or “homologous” as used herein is defined as the percentage of nucleotide residues in the homology arm that are identical to the nucleotide residues in the corresponding sequence on the target chromosome, after aligning the sequences and introducing gaps, if necessary, to achieve the maximum percent sequence identity. Alignment for purposes of determining percent nucleotide sequence homology can be achieved in various ways that are within the skill in the art, for instance, using publicly available computer software such as BLAST, BLAST-2, ALIGN, ClustalW2 or Megalign (DNASTAR) software. Those skilled in the art can determine appropriate parameters for aligning sequences, including any algorithms needed to achieve maximal alignment over the full length of the sequences being compared. In some embodiments, a nucleic acid sequence (e.g., DNA sequence), for example of a homology arm of a repair template, is considered “homologous” when the sequence is at least 70%, at least 75%, at least 80%, at least 85%, at least 90%, at least 91%, at least 92%, at least 93%, at least 94%, at least 95%, at least 96%, at least 97%, at least 98%, at least 99%, or more, identical to the corresponding native or unedited nucleic acid sequence (e.g., genomic sequence) of the host cell.

As used herein, a “vector” or “expression vector” is a replicon, which can be a nucleic acid construct designed for delivery to a host cell or for transfer between different host cells. As used herein, a vector can be viral or non-viral in origin in the final form. For the purpose of the present disclosure, a “vector” generally refers to synthetic, capsid-free AAV, for example a single-stranded (ss) synthetic vector or a nicked ceDNA vector. Accordingly, the term “vector” encompasses any genetic element that is capable of replication or expression when associated with the proper control elements and that can transfer gene sequences to cells. In some embodiments, a vector can be a recombinant vector or an expression vector. It is to be understood that the term “single-stranded (ss) synthetic vector” as used herein is meant to include a single-stranded AAV-like vector that may not have any viral sequence(s).

As used herein, the phrase “recombinant vector” means a vector that includes a heterologous nucleic acid sequence, or “transgene” that is capable of expression in vivo. It is to be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

As used herein, the term “expression vector” refers to a vector that directs expression of an RNA or polypeptide from sequences linked to transcriptional regulatory sequences on the vector. The sequences expressed will often, but not necessarily, be heterologous to the host cell. The expression vector may be a recombinant vector.

As used herein, the term “expression” refers to the cellular processes involved in producing RNA and proteins and as appropriate, secreting proteins, including where applicable, but not limited to, for example, transcription, transcript processing, translation and protein folding, modification and processing.

As used herein, the phrase “expression products” include RNA transcribed from a gene (e.g., transgene), and polypeptides obtained by translation of mRNA transcribed from a gene.

As used herein, the term “gene” means the nucleic acid sequence which is transcribed (DNA) to RNA in vitro or in vivo when operably linked to appropriate regulatory sequences. The gene may or may not include regions preceding and following the coding region, e.g., 5′ untranslated region (5′ UTR) or “leader” sequences and 3′ UTR or “trailer” sequences, as well as intervening sequences (introns) between individual coding segments (exons).

The terms “site-specific nuclease” or “sequence specific nuclease” as used herein refers to an enzyme capable of specifically recognizing and cleaving DNA sequences. The site-specific nuclease may be engineered. Examples of engineered site-specific nucleases include zinc finger nucleases (ZFNs), TAL effector nucleases (TALENs), and CRISPR/Cas-based systems, that use various natural and unnatural Cas enzymes.

The phrase “genetic disease” as used herein refers to a disease, partially or completely, directly or indirectly, caused by one or more abnormalities in the genome, especially a condition that is present from birth and can be treated by a single-stranded (ssDNA) molecule as described herein. The abnormality may be a mutation, an insertion or a deletion. The abnormality may affect the coding sequence of the gene or its regulatory sequence. The genetic disease may be, but not limited to phenylketonuria (PKU), melanoma, hemophilia A (clotting factor VIII (FVIII) deficiency) and hemophilia B (clotting factor IX (FIX) deficiency), cystic fibrosis, Huntington's chorea, familial hypercholesterolemia (LDL receptor defect), hepatoblastoma, Wilson's disease, congenital hepatic porphyria, inherited disorders of hepatic metabolism, Lesch Nyhan syndrome, sickle cell anemia, thalassaemias, xeroderma pigmentosum, Fanconi's anemia, retinitis pigmentosa, ataxia telangiectasia, Bloom's syndrome, retinoblastoma, and mucopolysaccharide storage diseases (e.g., Hurler syndrome (MPS Type I), Scheie syndrome (MPS Type I S), Hurler-Scheie syndrome (MPS Type I H-S), Hunter syndrome (MPS Type II), Sanfilippo Types A, B, C, and D (MPS Types III A, B, C, and D), Morquio Types A and B (MPS IVA and MPS IVB), Maroteaux-Lamy syndrome (MPS Type VI), Sly syndrome (MPS Type VII), hyaluronidase deficiency (MPS Type IX)), Niemann-Pick Disease Types A/B, C1 and C2, Fabry disease, Schindler disease, GM2-gangliosidosis Type II (Sandhoff Disease), Tay-Sachs disease, Metachromatic Leukodystrophy, Krabbe disease, Mucolipidosis Type I, II/III and IV, Sialidosis Types I and II, Glycogen Storage disease Types I and II (Pompe disease), Gaucher disease Types I, II and III, Fabry disease, cystinosis, Batten disease, Aspartylglucosaminuria, Salla disease, Danon disease (LAMP-2 deficiency), Lysosomal Acid Lipase (LAL) deficiency, neuronal ceroid lipofuscinoses (CLN1-8, INCL, and LINCL), sphingolipidoses, galactosialidosis. Also included in genetic disorders are amyotrophic lateral sclerosis (ALS), Parkinson's disease, Alzheimer's disease, Huntington's disease, spinocerebellar ataxia, spinal muscular atrophy, Friedreich's ataxia, Duchenne muscular dystrophy (DMD), Becker muscular dystrophies (BMD), dystrophic epidermolysis bullosa (DEB), ectonucleotide pyrophosphatase 1 deficiency, generalized arterial calcification of infancy (GACI), Leber Congenital Amaurosis (LCA, e.g., LCA10 [CEP290]), Stargardt macular dystrophy (ABCA4), or Cathepsin A deficiency.

As used herein, the term “increase,” “enhance,” “raise” (and like terms) generally refers to the act of increasing, either directly or indirectly, a concentration, level, function, activity, or behavior relative to the natural, expected, or average, or relative to a control condition.

As used herein, the term “suppress,” “decrease,” “interfere,” “inhibit” and/or “reduce” (and like terms) generally refers to the act of reducing, either directly or indirectly, a concentration, level, function, activity, or behavior relative to the natural, expected, or average, or relative to a control condition.

As used herein, the term “synthetic vector”, “single-stranded (ss) synthetic vector” and “synthetic production of a vector” refers to a vector and synthetic production methods thereof in a cell-free environment.

As used herein the term “comprising” or “comprises” is used in reference to compositions, methods, processes, and respective component(s) thereof, that are essential to the processes, methods or compositions, yet open to the inclusion of unspecified elements, whether essential or not. The use of “comprising” indicates inclusion rather than limitation.

The term “consisting of” refers to compositions, methods, processes, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the disclosure.

As used in this specification and the appended claims, the singular forms “a,” “an,” and “the” include plural references unless the context clearly dictates otherwise. Thus, for example, references to “the method” includes one or more methods, and/or steps of the type described herein and/or which will become apparent to those persons skilled in the art upon reading this disclosure and so forth. Similarly, the word “or” is intended to include “and” unless the context clearly indicates otherwise. Although methods and materials similar or equivalent to those described herein can be used in the practice or testing of this disclosure, suitable methods and materials are described below.

The abbreviation, “e.g.” is derived from the Latin exempli gratia and is used herein to indicate a non-limiting example. Thus, the abbreviation “e.g.” is synonymous with the term “for example.”

Other than in the operating examples, or where otherwise indicated, all numbers expressing quantities of ingredients or reaction conditions used herein should be understood as modified in all instances by the term “about.” The term “about” when used in connection with percentages can mean ±1%. The present disclosure is further explained in detail by the following examples, but the scope of the disclosure should not be limited thereto.

Groupings of alternative elements or embodiments of the disclosure disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the disclosure.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior disclosure or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting. It should be understood that this disclosure is not limited to the particular methodology, protocols, and reagents, etc., described herein and as such can vary. The terminology used herein is for the purpose of describing particular embodiments only and is not intended to limit the scope of the present disclosure, which is defined solely by the claims.

### II. Single-Stranded (ss) DNA Molecules

In some aspects, the present disclosure relates to single-stranded (ssDNA) molecules, e.g., synthetic ssDNA molecules, and the production thereof, e.g., from closed-ended DNA (ceDNA) and/or from a plasmid template using the methods described herein.

In some embodiments, the ssDNA molecule described herein is linear, single-stranded DNA molecule that is fully single-stranded along its entire length (that is, it contains no double-stranded regions).

### A. 3′ End Stem-Loop Structure

In some aspects, the disclosure provides a ssDNA molecule comprising at least one nucleic acid sequence of interest flanked by at least one stem-loop structure at the 3′ end. In some embodiments, the ssDNA molecule may further comprise at least one stem-loop structure at the 5′ end. As described herein, the stem-loop structure at the 3′ end may comprise a partial DNA duplex (e.g., with a free 3′-OH group) to prime replication or transcription. The partial DNA duplex functions, in part, to hold the stem-loop structure together.

According to some embodiments, the partial DNA duplex comprises between 4-500 nucleotides, for example between 4-10 nucleotides, between 4-25 nucleotides, between 4-50 nucleotides, between 4-100 nucleotides, between 4-200 nucleotides, between 4-300 nucleotides, between 4-400 nucleotides, between 20-25 nucleotides, between 20-50 nucleotides, between 20-100 nucleotides, between 20-200 nucleotides, between 20-300 nucleotides, between 20-400 nucleotides, between 20-500 nucleotides, between 50-100 nucleotides, between 50-200 nucleotides, between 50-300 nucleotides, between 50-400 nucleotides, between 50-500 nucleotides, 150-200 nucleotides, between 150-300 nucleotides, between 150-400 nucleotides, between 150-500 nucleotides, between 200-300 nucleotides, between 200-400 nucleotides, between 200-500 nucleotides, between 250-300 nucleotides, between 250-400 nucleotides, between 250-500 nucleotides, between 300-400 nucleotides, between 300-500 nucleotides, between 350-400 nucleotides, between 350-500 nucleotides, between 400-500 nucleotides, or between 450-500 nucleotides, and at least one loop on the 3′ end. According to some embodiments, the DNA duplex comprises at least 4, 5, 6, 7, 8, 9, 10, 20, 30, 40, 50, 60, 70, 80, 90, 100, 150, 200, 250, 300, 350, 400, 450 or 500 nucleotides, and at least one loop on the 3′ end.

According to some embodiments, the loop structure at the 3′ end comprises a minimum of between 3-500 unbound nucleotides, for example between 3-450 nucleotides, between 3-400 nucleotides, between 3-350 nucleotides, between 3-300 nucleotides, between 3-250 nucleotides, between 3-200 nucleotides, between 3-150 nucleotides, between 3-100 nucleotides, between 3-90 nucleotides, between 3-80 nucleotides, between 3-70 nucleotides, between 3-60 nucleotides, between 3-50 nucleotides, between 3-40 nucleotides, between 3-30 nucleotides, between 3-20 nucleotides, between 3-10 nucleotides, between 3-5 nucleotides, between 10-450 nucleotides, between 10-400 nucleotides, between 10-350 nucleotides, between 10-300 nucleotides, between 10-250 nucleotides, between 10-200 nucleotides, between 10-150 nucleotides, between 10-100 nucleotides, between 10-90 nucleotides, between 10-80 nucleotides, between 10-70 nucleotides, between 10-60 nucleotides, between 10-50 nucleotides, between 10-40 nucleotides, between 10-30 nucleotides, between 10-20 nucleotides, between 50-450 nucleotides, between 50-400 nucleotides, between 50-350 nucleotides, between 50-300 nucleotides, between 50-250 nucleotides, between 50-200 nucleotides, between 50-150 nucleotides, between 50-100 nucleotides, between 50-90 nucleotides, between 50-80 nucleotides, between 50-70 nucleotides, between 50-60 nucleotides, between 100-450 nucleotides, between 100-400 nucleotides, between 100-350 nucleotides, between 100-300 nucleotides, between 100-250 nucleotides, between 100-200 nucleotides, between 150-450 nucleotides, between 150-400 nucleotides, between 150-350 nucleotides, between 150-300 nucleotides, between 150-250 nucleotides, between 150-200 nucleotides, between 200-450 nucleotides, between 200-400 nucleotides, between 200-350 nucleotides, between 200-300 nucleotides, between 200-250 nucleotides, between 250-450 nucleotides, between 250-400 nucleotides, between 250-350 nucleotides, between 250-300 nucleotides, between 300-450 nucleotides, between 300-400 nucleotides, between 300-350 nucleotides, between 350-450 nucleotides, between 350-400 nucleotides, or between 400-450 nucleotides.

According to some embodiments, the stem portion of the stem-loop is 4-500 nucleotides in length and the loop portion of the stem-loop is 3-500 nucleotides in length. According to some embodiments, the stem portion of the stem-loop is 4-50 nucleotides in length and the loop portion of the stem-loop is 3-50 nucleotides in length. According to some embodiments, the stem portion of the stem-loop is 4-20 nucleotides in length and the loop portion of the stem-loop is 3-20 nucleotides in length. According to some embodiments, the stem portion of the stem-loop is 4-10 nucleotides in length and the loop portion of the stem-loop is 3-10 nucleotides in length.

According to some embodiments, the loop further comprises one or more nucleic acids or that are used to stabilize the ends. According to other embodiments, the loop further comprises one or more nucleic acids that may be employed in therapeutic methods. According to other embodiments, the loop further comprises one or more nucleic acids that may be employed in diagnostic methods. According to other embodiments, the loop further comprises one or more nucleic acids that that may be employed for research purposes.

According to some embodiments, the minimal nucleic acid structure that is necessary at the 3′ end of the ssDNA is any structure that loops back on itself, i.e., a hairpin structure. However, it is to be understood that a variety of structures are envisioned at the 3′ end, as long as there is at least one stem and one loop. For example, in some embodiments, the ssDNA described herein may comprise at least one stem-loop structure at the 3′ end. In some embodiments, the ssDNA may comprise at least two stem-loop structures at the 3′ end. In some embodiments, the ssDNA may comprise at least three stem-loop structures at the 3′ end. In some embodiments, the ssDNA may comprise at least four stem-loop structures at the 3′ end. In some embodiments, the ssDNA may comprise at least five stem-loop structures at the 3′ end.

According to some embodiments, the nucleotides at the 3′ end form a cruciform DNA structure. A DNA cruciform structure can be formed when both strands form a stem-loop structure at the same location in the molecule, and comprises a four-way junction and two closed hairpin-shaped points.

According to some embodiments, the nucleotides at the 3′ end form a hairpin DNA structure. Hairpin loop structures in nucleic acids consist of a base-paired stem structure and a loop sequence with unpaired or non-Watson-Crick-paired nucleotides.

According to some embodiments, the nucleotides at the 3′ end form a hammerhead DNA structure, made up of three base paired helices, separated by short linkers of conserved sequence.

According to some embodiments, the nucleotides at the 3′ end form a quadraplex DNA structure. G-quadruplexes are four-stranded DNA secondary structures (G4s) that form from certain guanine-rich sequences.

According to some embodiments, the nucleotides at the 3′ end form a bulged DNA structure.

According to some embodiments, the nucleotides at the 3′ end form a multibranched loop.

According to some embodiments, the nucleotides at the 3′ end do not form a 2 stem-loop structure.

According to some embodiments, the stem structure at the 3′ end comprises one or more nucleotides that are modified to be exonuclease resistant. According to some embodiments, the stem structure at the 3′ end comprises two or more, 3 or more, 4 or more, 5 or more, 6 or more, 7 or more, 8 or more, 9 or more, 10 or more, or 20 or more nucleotides that are modified to be exonuclease resistant.

According to some embodiments, the stem structure at the 3′ end comprises one or more phosphorothioate-modified nucleotides. According to some embodiments, the stem structure at the 3′ end comprises about 2 to about 12 phosphorothioate-modified nucleotides. According to some embodiments, the stem structure at the 3′ end comprises about 4 to about 10 phosphorothioate-modified nucleotides, e.g., about 4 to about 5, about 4 to about 6, about 4 to about 7, about 4 to about 8, about 4 to about 9, about 4 to about 10, about 5 to about 6, about 5 to about 7, about 5 to about 8, about 5 to about 9, about 5 to about 10, about 6 to about 7, about 6 to about 8, about 6 to about 9, about 6 to about 10, about 7 to about 8, about 7 to about 9, about 7 to about 10, about 8 to about 9, about 8 to about 10 or about 9 to about 10. According to some embodiments, the stem structure comprises more than 10 phosphorothioate-modified nucleotides.

According to some embodiments, the phosphorothioate-modified nucleotides are located adjacent to each other.

According to some embodiments, the one or more phosphorothioate-modified nucleotides of the 3′ end are resistant to exonuclease degradation. Boranophosphate modified DNA is also resistant to nuclease degradation, and may be considered as an alternative to phosphorothioate modification.

According to further embodiments, the stem structure may comprise at least one functional moiety. In one embodiment, the at least one functional moiety is an aptamer sequence. In further embodiments, the aptamer sequence has a high binding affinity to a nuclear localized protein.

According to some embodiments, the nucleotides in the loop are chemically modified with functional groups in order to alter their properties.

According to some embodiments, the loop further comprises one or more aptamers. According to some embodiments, the aptamer is identified from the Apta-index database of aptamers available to the public (aptagen.com/apta-index).

According to some embodiments, the loop further comprises one or more synthetic ribozymes.

According to some embodiments, the loop further comprises one or more antisense oligonucleotides (ASOs).

According to some embodiments, the loop further comprises one or more short-interfering RNAs (siRNAs).

According to some embodiments, the loop further comprises one or more antiviral nucleoside analogues (ANAs).

According to some embodiments, the loop further comprises one or more triplex forming oligonucleotides.

According to some embodiments, the loop further comprises one or more gRNAs or gDNAs.

According to some embodiments, the loop further comprises one or more molecular probes, for example nucleic acid based fluorescent probes.

According to some embodiments, “click” azide-alkyne cycloaddition (Kolb et al., Angew. Chem. Int. Ed. Engl. 2001, 40, 2004-2021) is used to modify the nucleotides in the loop. Click chemistry was developed to join together organic molecules under mild conditions in the presence of a diverse range of functional groups. Most click-mediated modifications are performed on the nitrogenous bases by introducing novel base analogues, attaching fluorophores or isotopic elements for molecular imaging, forming inter-strand linkages between oligonucleotides, and for the bioconjugation of molecules. The best example of click chemistry is the Cu^{I} catalyzed version of Huisgen's [3+2] azide-alkyne cycloaddition reaction (Angew. Chem., Int. Ed. 1963, 2, 633-645), discovered independently by Sharpless and Meldal (the CuAAC reaction) (Angew. Chem., Int. Ed. 2002, 41, 2596-2599).

According to some embodiments, the introduction of active amino or thiol groups into synthesized oligonucleotides provides acceptors for, e.g., subsequent chemical fluorescent labeling.

According to some embodiments, the stem-loop structure may comprise alternative or modified nucleotides, including, but not limited to, ribonucleic acids (RNA), peptide-nucleic acids (PNA), locked nucleic acids (LNA). According to some embodiments, the loop portion of the stem-loop structure may comprise a chemical structure that does not comprise nucleic acids.

According to some embodiments, the ssDNA molecule does not comprise any virally-derived sequences.

Difference from Known ITR Structures

As is known in the art, typical AAV ITR structures comprise a palindromic double-stranded T-shaped hairpin structure, in which the double-stranded A-A′ region forms the stem, and the double-stranded B-B′ and C-C′ regions form the cross-arms of the T-shaped structure (see, for example, Ling et al., J. Virology, 89 (2): 952-961, 2015). The other nucleotides of the typical AAV ITR remain single stranded and are referred to as the single-stranded D(−) sequence (on the 3′ end of the ITR) and the single-stranded D(+) sequence (on the 5′ end of the ITR). Once in cells, the single-stranded regions of the D(+) region and D(−) region undergo second-strand DNA synthesis to turn them into double-stranded D and D′ regions. Thus, when generically used herein, the term “D region” refers either to the single-stranded D(−) and/or D(+) region, or the double-stranded D and/or D′ region, as is appropriate in the context of the disclosure.

Prior to the instant invention, it had been demonstrated that removal of both the ssD(+) and ssD(−) region from the AAV ITR impairs rescue, replication and encapsidation of AAV DNA (see, e.g., Wang et al., J. Mol. Biol., 250:573-580, 1995; Wang et al., J. Virol., 70:1668-1677, 1996; and Wang et al., J. Virol., 71:3077-3082, 1997), and it was thought by one of ordinary skill in the art that at least one of the D(+) or D(−) single-stranded regions was absolutely necessary for replication and encapsidation of AAV and deletion of ssD(+) or ssD(−) also may negatively affect expression of AAV DNA as they were thought to contain one or more transcription factor binding sites (see, e.g., Ling et al., J. Virology, 89 (2): 952-961, 2015; WO2016081927A2).

However, the inventors of the instant invention have surprisingly discovered that deletion of both the D(+) and D(−) regions from the stem-loop structures of the disclosed single-stranded DNA molecules results in functional single-stranded DNA (ssDNA).

Accordingly, in some embodiments, the ssDNA does not comprise a D(−) region or a D(+) region that would be present in a wild-type AAV ITR. In some embodiments, the at least one stem-loop structure at the 3′ end of the ssDNA do not comprise a single-stranded D(−) region. In other embodiments, the at least one stem-loop structure at the 3′ end of the ssDNA molecule does not comprise any of the A, A′, B, B′, C, C′, and/or D(−) regions that would be present in a wild-type AAV ITR.

According to some embodiments, the at least one stem-loop structure at the 3′ end does not comprise a rep binding element (RBE) that would be present in a wild-type ITR. According to some embodiments, the at least one stem-loop structure at the 3′ end does not comprise a terminal resolution site (trs) that would be present in a wild-type ITR.

In some embodiments, the at least one stem loop structure at the 3′ end is devoid of any viral capsid protein coding sequences.

In some embodiments, the nucleotides at the 3′ end of the ssDNA do not form an AAV ITR structure.

### B. 5′ End Stem-Loop Structure

In some embodiments, the ssDNA molecule comprising at least one nucleic acid sequence of interest flanked by at least one stem-loop structure at the 3′ end further comprises a 5′ end, comprising at least one stem-loop structure. As described herein, the stem-loop structure at the 5′ end may comprise a partial DNA duplex.

According to some embodiments, the partial DNA duplex comprises between 4-500 nucleotides, for example between 4-10 nucleotides, between 4-25 nucleotides, between 4-50 nucleotides, between 4-100 nucleotides, between 4-200 nucleotides, between 4-300 nucleotides, between 4-400 nucleotides, between 20-25 nucleotides, between 20-50 nucleotides, between 20-100 nucleotides, between 20-200 nucleotides, between 20-300 nucleotides, between 20-400 nucleotides, between 20-500 nucleotides, between 50-100 nucleotides, between 50-200 nucleotides, between 50-300 nucleotides, between 50-400 nucleotides, between 50-500 nucleotides, 150-200 nucleotides, between 150-300 nucleotides, between 150-400 nucleotides, between 150-500 nucleotides, between 200-300 nucleotides, between 200-400 nucleotides, between 200-500 nucleotides, between 250-300 nucleotides, between 250-400 nucleotides, between 250-500 nucleotides, between 300-400 nucleotides, between 300-500 nucleotides, between 350-400 nucleotides, between 350-500 nucleotides, between 400-500 nucleotides, or between 450-500 nucleotides, and at least one loop on the 3′ end. According to some embodiments, the DNA duplex comprises at least 4, 5, 6, 7, 8, 9, 10, 20, 30, 40, 50, 60, 70, 80, 90, 100, 150, 200, 250, 300, 350, 400, 450 or 500 nucleotides, and at least one loop on the 5′ end.

According to some embodiments, a loop structure at the 5′ end comprises a minimum of between 3-500 unbound nucleotides, for example between 3-450 nucleotides, between 3-400 nucleotides, between 3-350 nucleotides, between 3-300 nucleotides, between 3-250 nucleotides, between 3-200 nucleotides, between 3-150 nucleotides, between 3-100 nucleotides, between 3-90 nucleotides, between 3-80 nucleotides, between 3-70 nucleotides, between 3-60 nucleotides, between 3-50 nucleotides, between 3-40 nucleotides, between 3-30 nucleotides, between 3-20 nucleotides, between 3-10 nucleotides, between 3-5 nucleotides, between 10-450 nucleotides, between 10-400 nucleotides, between 10-350 nucleotides, between 10-300 nucleotides, between 10-250 nucleotides, between 10-200 nucleotides, between 10-150 nucleotides, between 10-100 nucleotides, between 10-90 nucleotides, between 10-80 nucleotides, between 10-70 nucleotides, between 10-60 nucleotides, between 10-50 nucleotides, between 10-40 nucleotides, between 10-30 nucleotides, between 10-20 nucleotides, between 50-450 nucleotides, between 50-400 nucleotides, between 50-350 nucleotides, between 50-300 nucleotides, between 50-250 nucleotides, between 50-200 nucleotides, between 50-150 nucleotides, between 50-100 nucleotides, between 50-90 nucleotides, between 50-80 nucleotides, between 50-70 nucleotides, between 50-60 nucleotides, between 100-450 nucleotides, between 100-400 nucleotides, between 100-350 nucleotides, between 100-300 nucleotides, between 100-250 nucleotides, between 100-200 nucleotides, between 150-450 nucleotides, between 150-400 nucleotides, between 150-350 nucleotides, between 150-300 nucleotides, between 150-250 nucleotides, between 150-200 nucleotides, between 200-450 nucleotides, between 200-400 nucleotides, between 200-350 nucleotides, between 200-300 nucleotides, between 200-250 nucleotides, between 250-450 nucleotides, between 250-400 nucleotides, between 250-350 nucleotides, between 250-300 nucleotides, between 300-450 nucleotides, between 300-400 nucleotides, between 300-350 nucleotides, between 350-450 nucleotides, between 350-400 nucleotides, or between 400-450 nucleotides.

According to some embodiments, the stem portion of the stem-loop is 4-500 nucleotides in length and the loop portion of the stem-loop is 3-500 nucleotides in length. According to some embodiments, the stem portion of the stem-loop is 4-50 nucleotides in length and the loop portion of the stem-loop is 3-50 nucleotides in length. According to some embodiments, the stem portion of the stem-loop is 4-20 nucleotides in length and the loop portion of the stem-loop is 3-20 nucleotides in length. According to some embodiments, the stem portion of the stem-loop is 4-10 nucleotides in length and the loop portion of the stem-loop is 3-10 nucleotides in length.

According to some embodiments, the loop further comprises one or more nucleic acids or that are used to stabilize the ends. According to other embodiments, the loop further comprises one or more nucleic acids that may be employed in therapeutic methods. According to other embodiments, the loop further comprises one or more nucleic acids that may be employed in diagnostic methods. According to other embodiments, the loop further comprises one or more nucleic acids that that may be employed for research purposes.

According to some embodiments, the minimal nucleic acid structure that is necessary at the 5′ end of the ssDNA is any structure that loops back on itself, i.e., a hairpin structure. However, it is to be understood that a variety of structures are envisioned at the 5′ end, as long as there is at least one stem and one loop. For example, in some embodiments, the ssDNA described herein may comprise at least one stem-loop structure at the 5′ end. In some embodiments, the ssDNA may comprise at least two stem-loop structures at the 5′ end. In some embodiments, the ssDNA may comprise at least three stem-loop structures at the 5′ end. In some embodiments, the ssDNA may comprise at least four stem-loop structures at the 5′ end. In some embodiments, the ssDNA may comprise at least five stem-loop structures at the 5′ end.

According to some embodiments, the nucleotides at the 5′ end form a cruciform DNA structure. A DNA cruciform structure can be formed when both strands form a stem-loop structure at the same location in the molecule, and comprises a four-way junction and two closed hairpin-shaped points.

According to some embodiments, the DNA structure at the 5′ end is the same as the DNA structure at the 3′ end. According to some embodiments, the DNA structure at the 5′ end is different from the DNA structure at the 3′ end.

For example, in some embodiments, the ssDNA described herein may comprise at least one stem-loop structure at the 5′ end. According to some embodiments, ssDNA may comprise at least two stem-loop structures at the 5′ end. According to some embodiments, the ssDNA may comprise at least three stem-loop structures at the 5′ end. According to some embodiments, the ssDNA may comprise at least four stem-loop structures at the 5′ end. According to some embodiments, the ssDNA may comprise at least five stem-loop structures at the 5′ end.

According to some embodiments, the nucleotides at the 5′ end form a cruciform DNA structure.

According to some embodiments, the nucleotides at the 5′ end form a hairpin structure.

According to some embodiments, the nucleotides at the 5′ end form a hammerhead structure.

According to some embodiments, the nucleotides at the 5′ end form a quadraplex structure.

According to some embodiments, the nucleotides at the 5′ end form a bulged structure.

According to some embodiments, the nucleotides at the 5′ end form a multibranched loop.

According to some embodiments, the nucleotides at the 5′ end do not form a 2 stem-loop structure.

According to some embodiments, the stem structure at the 5′ end comprises one or more nucleotides that are modified to be exonuclease resistant. According to some embodiments, the stem structure at the 5′ end comprises two or more, 3 or more, 4 or more, 5 or more, 6 or more, 7 or more, 8 or more, 9 or more, 10 or more, or 20 or more nucleotides that are modified to be exonuclease resistant

According to some embodiments, the stem structure comprises one or more phosphorothioate-modified nucleotides. According to some embodiments, the stem structure comprises about 2 to about 12 phosphorothioate-modified nucleotides. According to some embodiments, the stem structure comprises about 4 to about 10 phosphorothioate-modified nucleotides, e.g., about 4 to about 5, about 4 to about 6, about 4 to about 7, about 4 to about 8, about 4 to about 9, about 4 to about 10, about 5 to about 6, about 5 to about 7, about 5 to about 8, about 5 to about 9, about 5 to about 10, about 6 to about 7, about 6 to about 8, about 6 to about 9, about 6 to about 10, about 7 to about 8, about 7 to about 9, about 7 to about 10, about 8 to about 9, about 8 to about 10 or about 9 to about 10. According to some embodiments, the stem structure comprises more than 10 phosphorothioate-modified nucleotides.

According to some embodiments, the phosphorothioate-modified nucleotides are located adjacent to each other. According to some embodiments, the one or more phosphorothioate-modified nucleotides of the are resistant to exonuclease degradation.

According to some embodiments, the loop further comprises one or more nucleic acids or that are used to stabilize the ends. According to other embodiments, the loop further comprises one or more nucleic acids that may be employed in therapeutic methods. According to other embodiments, the loop further comprises one or more nucleic acids that may be employed in diagnostic methods. According to other embodiments, the loop further comprises one or more nucleic acids that that may be employed for research purposes.

According to some embodiments, the nucleotides in the loop are chemically modified with functional groups in order to alter their properties.

According to some embodiments, the loop further comprises one or more aptamers. According to some embodiments, the aptamer is identified from the Apta-index database of aptamers available to the public (aptagen.com/apta-index).

According to some embodiments, the loop further comprises one or more synthetic ribozymes.

According to some embodiments, the loop further comprises one or more antisense oligonucleotides (ASOs).

According to some embodiments, the loop further comprises one or more short-interfering RNAs (siRNAs).

According to some embodiments, the loop further comprises one or more antiviral nucleoside analogues (ANAs).

According to some embodiments, the loop further comprises one or more triplex forming oligonucleotides.

According to some embodiments, the loop further comprises one or more gRNAs or gDNAs.

According to some embodiments, the loop further comprises one or more molecular probes, for example nucleic acid based fluorescent probes.

According to some embodiments, “click” azide-alkyne cycloaddition (Kolb et al., Angew. Chem. Int. Ed. Engl. 2001, 40, 2004-2021) is used to modify the nucleotides in the loop. Click chemistry was developed to join together organic molecules under mild conditions in the presence of a diverse range of functional groups. Most click-mediated modifications are performed on the nitrogenous bases by introducing novel base analogues, attaching fluorophores or isotopic elements for molecular imaging, forming inter-strand linkages between oligonucleotides, and for the bioconjugation of molecules. The best example of click chemistry is the Cu^{I} catalyzed version of Huisgen's [3+2] azide-alkyne cycloaddition reaction (Angew. Chem., Int. Ed. 1963, 2, 633-645), discovered independently by Sharpless and Meldal (the CuAAC reaction) (Angew. Chem., Int. Ed. 2002, 41, 2596-2599).

According to some embodiments, the introduction of active amino or thiol groups into synthesized oligonucleotides provides acceptors for, e.g., subsequent chemical fluorescent labeling.

According to some embodiments, the stem-loop structure may comprise alternative or modified nucleotides, including, but not limited to, ribonucleic acids (RNA), peptide-nucleic acids (PNA), locked nucleic acids (LNA). According to some embodiments, the loop portion of the stem-loop structure may comprise a chemical structure that does not comprise nucleic acids.

Difference from Known ITR Structures

As is known in the art, typical AAV ITR structures comprise a palindromic double-stranded T-shaped hairpin structure, in which the double-stranded A-A′ region forms the stem, and the double-stranded B-B′ and C-C′ regions form the cross-arms of the T-shaped structure (see, for example, Ling et al., J. Virology, 89(2):952-961, 2015; WO2016081927A2). The other nucleotides of the typical AAV ITR remain single stranded and are referred to as the single-stranded D(−) sequence (on the 3′ end of the ITR) and the single-stranded D(+) sequence (on the 5′ end of the ITR). Once in cells, the single-stranded regions of the D(+) region and D(−) region undergo second-strand DNA synthesis to turn them into double-stranded D and D′ regions.

Prior to the instant invention, it had been demonstrated that removal of both the D(+) and D(−) region from the AAV ITR impairs rescue, replication and encapsidation of AAV DNA (see, e.g., Wang et al., J. Mol. Biol., 250:573-580, 1995; Wang et al., J. Virol., 70:1668-1677, 1996; and Wang et al., J. Virol., 71:3077-3082, 1997), and it was thought by one of ordinary skill in the art that at least one of the D(+) or D(−) single-stranded regions was absolutely necessary for replication and encapsidation of AAV and deletion of ssD(+) or ssD(−) also may negatively affect expression of AAV DNA as they were thought to contain one or more transcription factor binding sites (see, e.g., Ling et al., J. Virology, 89 (2): 952-961, 2015; WO2016081927A2).

However, the inventors of the instant invention have surprisingly discovered that deletion of both the ssD(+) and ssD(−) regions from the stem-loop structures of the disclosed single-stranded DNA molecules results in functional single-stranded DNA (ssDNA).

Accordingly, in some embodiments, the at least one stem-loop structure of the ssDNA does not comprise a ssD(−) region or a ssD(+) region that would be present in a wild-type AAV ITR. In some embodiments, the at least one stem-loop structure at the 5′ end of the ssDNA do not comprise a single-stranded D(+) region. In other embodiments, the at least one stem-loop structure at the 5′ end of the ssDNA molecule does not comprise any of the A, A′, B, B′, C, C′, and/or D(+) regions that would be present in a wild-type AAV ITR.

According to some embodiments, the at least one stem-loop structure at the 5′ end does not comprise a rep binding element (RBE) that would be present in a wild-type ITR. According to some embodiments, the at least one stem-loop structure at the 5′ end does not comprise a terminal resolution site (trs) that would be present in a wild-type ITR.

In some embodiments, the at least one stem loop structure at the 5′ end is devoid of any viral capsid protein coding sequences.

In some embodiments, the nucleotides at the 5′ end of the ssDNA do not form an AAV ITR structure.

### C. Transgenes

The single-stranded DNA (ssDNA) molecules described herein have no packaging constraints imposed by the limiting space within the viral capsid. This permits the insertion of one or more genetic elements, e.g., a single-stranded enhancer, a single-stranded intron, a single-stranded posttranscriptional regulatory element, a single-stranded polyadenylation signal, and a single-stranded regulatory switch, large transgenes, multiple transgenes etc.

According to some embodiments, the transgene, e.g., nucleic acid sequence of interest, further comprises at least one single-stranded promoter linked to the at least one nucleic acid sequence of interest.

In other aspects of the disclosure, the single-stranded transgene cassettes find use in gene editing applications, as described in more detail herein.

According to some embodiments, the nucleic acid sequence of interest (also referred to as a transgene herein) encodes a protein that is either absent, inactive, or insufficient activity in the recipient subject or a gene that encodes a protein having a desired biological or a therapeutic effect. The transgene can encode a gene product that can function to correct the expression of a defective gene or transcript. In principle, the expression cassette can include any gene that encodes a protein, polypeptide or RNA that is either reduced or absent due to a mutation or which conveys a therapeutic benefit when overexpressed is considered to be within the scope of the disclosure.

The nucleic acid sequence of interest can comprise any sequence that is useful for treating a disease or disorder in a subject. A ssDNA molecule can be used to deliver and express any gene of interest in the subject, which includes but are not limited to, nucleic acids encoding polypeptides, or non-coding nucleic acids (e.g., RNAi, miRs etc.), as well as exogenous genes and nucleotide sequences, including virus sequences in a subjects' genome, e.g., HIV virus sequences and the like. In some embodiments, ssDNA molecules disclosed herein are used for therapeutic purposes (e.g., for medical, diagnostic, or veterinary uses). In certain embodiments, ssDNA molecules are useful to express any gene of interest in the subject, which includes one or more polypeptides, peptides, ribozymes, peptide nucleic acids, siRNAs, RNAis, antisense oligonucleotides, antisense polynucleotides, or RNAs (coding or non-coding; e.g., siRNAs, shRNAs, micro-RNAs, mRNA or gRNA, and their antisense counterparts (e.g., antagoMiR)), antibodies, antigen binding fragments, or any combination thereof.

Sequences can be codon optimized for the target host cell. As used herein, the term “codon optimized” or “codon optimization” refers to the process of modifying a nucleic acid sequence for enhanced expression in the cells of the vertebrate of interest, e.g., mouse or human, by replacing at least one, more than one, or a significant number of codons of the native sequence (e.g., a prokaryotic sequence) with codons that are more frequently or most frequently used in the genes of that vertebrate. Various species exhibit particular bias for certain codons of a particular amino acid. Typically, codon optimization does not alter the amino acid sequence of the original translated protein. Optimized codons can be determined using e.g., Aptagen's GENEFORGE® codon optimization and custom gene synthesis platform (Aptagen, Inc., 2190 Fox Mill Rd. Suite 300, Herndon, Va. 20171) or another publicly available database.

In some embodiments, a transgene expressed by the ssDNA molecules is a therapeutic gene. In some embodiments, a therapeutic gene is an antibody, or antibody fragment, or antigen-binding fragment thereof, e.g., a neutralizing antibody or antibody fragment and the like.

In particular, a therapeutic gene is one or more therapeutic agent(s), including, but not limited to, for example, protein(s), polypeptide(s), peptide(s), enzyme(s), antibodies, antigen binding fragments, as well as variants, and/or active fragments thereof, for use in the treatment, prophylaxis, and/or amelioration of one or more symptoms of a disease, dysfunction, injury, and/or disorder. Exemplary therapeutic genes are described herein in the section entitled “Method of Treatment”.

According to any of the above aspects and embodiments, the ssDNA molecules are synthetically produced.

According to any of the above aspects and embodiments, the ssDNA molecules are devoid of any viral capsid protein coding sequences.

According to any of the above aspects the DNA is peptide nucleic acid (PNA) are synthetic mimics of DNA.

### D. Promoters

In some embodiments, an ssDNA molecule produced by the methods described herein comprises a promoter (described in more detail below), wherein the promoter comprises a transcription start site (TSS). In some embodiments, an ssDNA molecule produced by the methods described herein comprises an enhancer.

In some embodiments, the promoter, TSS, and/or enhancer are single-stranded in an ssDNA molecule produced by the methods described herein. In some embodiments, the promoter, TSS, and/or enhancer are double-stranded in an ssDNA molecule produced by the methods described herein.

Accordingly, in some embodiments, the double-stranded region comprising the promoter, enhancer, and/or TSS is at least 10 base pairs, at least 20 base pairs, at least 30 base pairs, at least 40 base pairs, at least 50 base pairs, at least 60 base pairs, at least 70 base pairs, at least 80 base pairs, at least 90 base pairs, at least 100 base pairs, at least 110 base pairs, at least 120 base pairs, at least 130 base pairs, at least 140 base pairs, at least 150 base pairs, at least 160 base pairs, at least 170 base pairs, at least 180 base pairs, at least 190 base pairs, at least 200 base pairs, at least 220 base pairs, at least 240 base pairs, at least 260 base pairs, at least 280 base pairs, at least 300 base pairs, at least 320 base pairs, at least 340 base pairs, at least 360 base pairs, at least 380 base pairs, at least 400 base pairs, at least 420 base pairs, at least 440 base pairs, at least 460 base pairs, at least 480 base pairs, at least 500 base pairs, at least 550 base pairs, at least 600 base pairs, at least 650 base pairs, at least 700 base pairs, at least 750 base pairs, at least 800 base pairs, at least 850 base pairs, at least 900 base pairs, at least 950 base pairs, at least 1000 base pairs, at least 1100 base pairs, at least 1200 base pairs, at least 1300 base pairs, at least 1400 base pairs, or at least 1500 base pairs in length.

In some embodiments, the double-stranded region comprising the promoter, enhancer, and/or TSS is less than 1500 base pairs, less than 1400 base pairs, less than 1300 base pairs, less than 1200 base pairs, less than 1100 base pairs, less than 1000 base pairs, less than 950 base pairs, less than 900 base pairs, less than 850 base pairs, less than 800 base pairs, less than 750 base pairs, less than 700 base pairs, less than 650 base pairs, less than 600 base pairs, less than 550 base pairs, less than 500 base pairs, less than 480 base pairs, less than 460 base pairs, less than 440 base pairs, less than 420 base pairs, less than 400 base pairs, less than 380 base pairs, less than 360 base pairs, less than 340 base pairs, less than 320 base pairs, less than 300 base pairs, less than 280 base pairs, less than 260 base pairs, less than 240 base pairs, less than 220 base pairs, less than 200 base pairs, less than 190 base pairs, less than 180 base pairs, less than 170 base pairs, less than 160 base pairs, less than 150 base pairs, less than 140 base pairs, less than 130 base pairs, less than 120 base pairs, less than 110 base pairs, less than 100 base pairs, less than 90 base pairs, less than 80 base pairs, less than 70 base pairs, less than 60 base pairs, less than 50 base pairs, less than 40 base pairs, or less than 30 base pairs in length.

In some embodiments, the double-stranded region comprising the promoter, enhancer, and/or TSS is about 30-1500 base pairs in length, about 40-1400 base pairs in length, about 50-1300 base pairs in length, about 60-1200 base pairs in length, about 70-1100 base pairs in length, about 80-1000 base pairs in length, about 90-900 base pairs in length, about 90-900 base pairs in length, about 100-800 base pairs in length, about 110-700 base pairs in length, about 120-600 base pairs in length, about 130-500 base pairs in length, about 140-400 base pairs in length, about 150-300 base pairs in length, about 160-200 base pairs in length, about 1381 base pairs in length, or about 499 base pairs in length.

### E. Apatmers

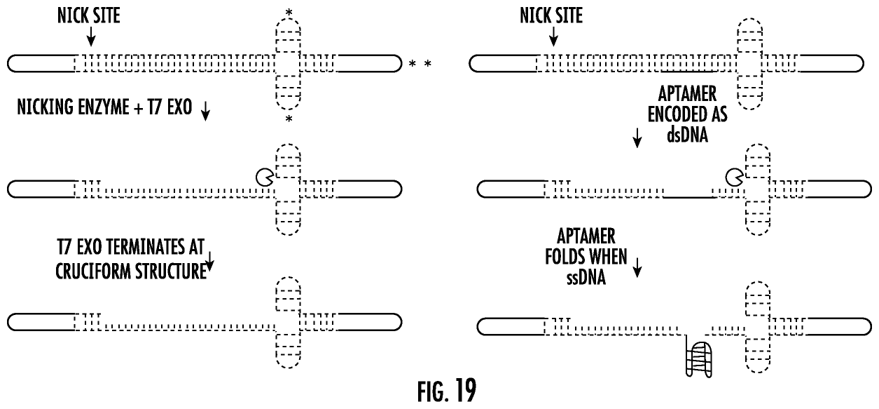

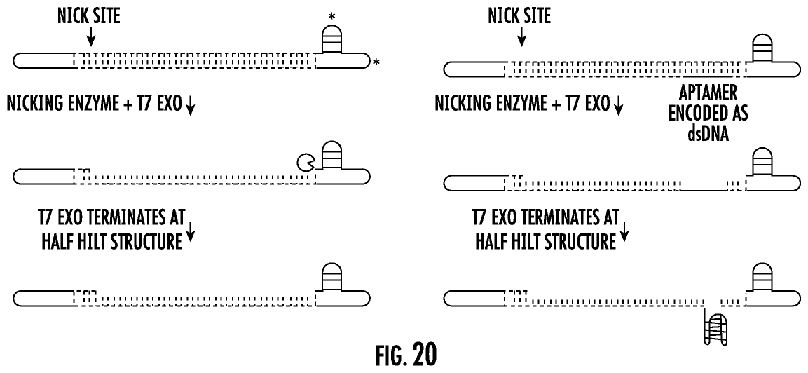

In some embodiments, an ssDNA molecule produced by the methods described herein comprises an aptamer, which are described in more detail throughout the disclosure. In some embodiments, an aptamer may be located in a 3′ and/or 5′ stem-loop structure of an ssDNA molecule produced by the methods described herein. In some embodiments, an aptamer may be located within or adjacent to a nucleic acid sequence of interest. In some embodiments, an aptamer may be encoded in a double-stranded ceDNA molecule, and the aptamer may only fold into a secondary structure after one strand of the double-stranded ceDNA molecule is removed to produce an ssDNA molecule (see, for example, FIG. 19, right, and FIG. 20, right). In some embodiments an aptamer is a CH4-1 aptamer.

### III. Closed-Ended DNA (ceDNA) Intermediate Molecules

As described herein, a cell-free, enzymatic method is used to generate a synthetic double-stranded closed-ended DNA (ceDNA) intermediate molecule. In one embodiment, the disclosure provides an isolated closed-ended DNA (ceDNA) construct comprising a double-stranded transgene cassette comprising at least one double-stranded transgene; and a first inverted terminal repeat (ITR) and an optional second ITR that each flanks the at least one double-stranded transgene; wherein at least one of the first ITR and the optional second ITR comprises one or more phosphorothioate-modified nucleotides.

According to embodiments of the disclosure, the double-stranded transgene cassette further comprises at least one double-stranded promoter operably linked to the at least one double-stranded transgene to control expression of the at least one double-stranded transgene. In further embodiments, the double-stranded transgene cassette further comprises one or more genetic elements selected from the group consisting of a double-stranded enhancer, a double-stranded intron, a double-stranded posttranscriptional regulatory element, a double-stranded polyadenylation signal, and a double-stranded regulatory switch. According to other further embodiments, the at least one double-stranded transgene is a promoterless double-stranded transgene. As described herein the at least one double-stranded transgene is a double-stranded donor sequence; and the double-stranded transgene cassette further comprises a double-stranded 5′ homology arm and a double-stranded 3′ homology arm flanking the double-stranded donor sequence. According to some embodiments, the double-stranded 5′ homology arm and the double-stranded 3′ homology arm are each between about 10 to 2000 nt in length, for example about 100 to 2000 nt in length or about 1000 to 2000 nt in length, or about 10 to 1000 nt in length, for example about 100 to 1000 nt in length or about 10 to 500 nt in length, about 50 to 500 nt in length or about 100 to 500 nt in length, about 10 to 50 nt in length, about 50 to 500 nt in length or about 500 to 1000 nt in length, about 500 to 1500 nt in length, about 1500 to 2000 nt in length, about 2 to 1000 nt in length, about 2 to 500 nt in length, about 2 to 100 nt in length, or about 2 to 50 nt in length.

According to some embodiments, the at least one double-stranded transgene is a double-stranded donor sequence; and the double-stranded transgene cassette is devoid of a single-stranded 5′ homology arm and a single-stranded 3′ homology arm. Further, in some embodiments, the double-stranded transgene cassette is cleavable and further comprises at least a first double-stranded guide RNA (gRNA) target sequence (TS); at least a first double-stranded protospacer adjacent motif (PAM); at least a second double-stranded gRNA TS; and at least a second double-stranded PAM.

Owing to the fact that the single-stranded DNA (ssDNA) according to embodiments of the present disclosure are derived from double-stranded DNA (dsDNA) intermediates, the physical attributes of the ds ceDNA vectors are also present in the single-stranded DNA (ssDNA) molecules, including, e.g., the presence of the at least one functional moiety as such as an aptamer sequence, e.g., having a high binding affinity to a nuclear localized protein or a fluorophore chemically conjugated to the ITR oligonucleotides.

The single-stranded DNA (ssDNA) molecules and ds DNA constructs (e.g., ds ceDNA) produced using the synthetic process as described herein have no packaging constraints imposed by the limiting space within the viral capsid. This permits the insertion of control elements, e.g., regulatory switches as disclosed herein, large transgenes, multiple transgenes etc.

### A. Endonuclease Recognition Nucleotide Sequences

According to some embodiments, the ceDNA construct comprises a nickase recognition sequence (“nick site”) for an endonuclease, e.g., a nicking endonuclease. In one embodiment, the dsDNA construct comprise a terminal resolution site (trs) sequence of an AAV ITR that contains a nick site for an endonuclease. According to some embodiments, the dsDNA construct comprises one or more recognition nucleotide sequences of one or more nicking endonucleases that are each independently selected from Nb.BbvCI, Nb.BsmI, Nb.BsrDI, Nb.BssSI, Nb.BtsI, Nt.AlwI, Nt.BbvCI, Nt.BsmI, Nt.BspQI, Nt.BstNBI, Nt. CviPII, and an isoschizomer of any of the foregoing. According to a further embodiment, the one or more recognition nucleotide sequences comprise any one or more of the following sequences shown in Table 1 below:

TABLE 1

| Sequence | Nicking Endonuclease |
|---|---|
| 5′-GCTGAGG-3′ | (Nb.BbvCI) |
| 5′ NGCATTC-3′ | (Nb.BsmI) N can be |
| | G, C, A, or T |
| 5′-NNCATTGC-3′ | (Nb.BsrDI) |
| 5′-CTCGTG-3′ | (Nb.BssSI) |
| 5′-NNCACTGC-3′ | (Nb.BtsI) |
| 5′-GGATCNNNNN-3′ | (Nt.AlwI) |
| 5′-CCTCAGC-3′ | (Nt.BbvCI) |
| 5′-GTCTCNN-3′ | (Nt.BsmI) |
| 5′-GTCTCNN-3′ | (Nt.BsmI) |
| 5′-GCTCTTCN-3′ | (Nt.BspQI) |
| 5′-GAGTCNNNNN-3′ | (Nt.BstNBI) |
| 5′-CCD-3′ | (Nt.CviPII) D |
| | can be A or G or T |

According to some embodiments, the one or more recognition nucleotide sequences are each an engineered sequence. According to further embodiments, the one or more recognition nucleotide sequences each comprise one or more nick sites of the one or more nicking endonucleases.

According to some embodiments where an ITR comprising a terminal resolution site (trs) the one or more nick sites are about 0 to about 20 nucleotides downstream of the (trs), for example, about 0, 1, 2, 3, 4, 5, 6, 7, 8, 9, 10, 11, 12, 13, 14, 15, 16, 17, 18 19, or 20 nucleotides downstream of the terminal resolution site (trs), or for example about 0 to about 15, about 0 to 10, about 0 to 5, about 5 to 15, about 10 to 20, about 15 to 20, about 10 to 20, about 5 to 20 nucleotides downstream of the terminal resolution site (trs). According to some embodiments, there is just one nick site that serves as the exonuclease entry site. In some embodiments where an ITR does not comprise a trs, the nick site may be in a stem region upstream of an expression cassette. In some embodiments, the nick site is at least about 0, 1, 2, 3, 4, 5, 6, 7, 8, 9, 10, 11, 12, 13, 14, 15, 16, 17, 18 19, 20, 25, 30, 35, or 40, nucleotides upstream of an expression cassette.

According to some embodiments, the dsDNA construct comprises one or more recognition nucleotide sequences of Nb.BbvCI or an isoschizomer thereof. According to some embodiments, the dsDNA construct comprises a single recognition nucleotide sequence of Nb.BbvCI or an isoschizomer thereof. According to some embodiments, the dsDNA construct comprises one or more recognition nucleotide sequences of Nb.BtsI or an isoschizomer thereof. According to some embodiments, the dsDNA construct comprises a single recognition nucleotide sequence of Nb.BtsI or an isoschizomer thereof. According to some embodiments, the dsDNA construct comprises one or more recognition nucleotide sequences of Endonuclease V or an isoschizomer thereof.

According to some embodiments, the double-stranded ceDNA molecule comprises at least one deoxyinosine residue. According to some embodiments, the deoxyinosine residue is present in the stem-loop structure at the 3′ end, two bases upstream of a desired nick site.

According to some embodiments, the deoxyinosine modification is present at a position of -1i, -2i, -3i, -4i, -5i, -6i, -7i, -8i, -9i, or -10i relative to the 3′ end of the 3′ITR.

According to some embodiments, the deoxyinosine modification is present at a position of -1i, -2i, -5i, or -7i relative to the 3′ end of the 3′ITR.

According to some embodiments, the deoxyinosine residue is present at the position of -1i, -2i, -5i, or -7i relative to SEO ID NO: 7, shown below:

(SEQ ID NO: 7)
CTCACTGAGGCCGCCCGGGCAAAGCCCGGGCGTCGGGCGA
CCTTTGGTCGCCCGGCCTCAG

According to some embodiments, the position of the inosine modification affects the stability of the secondary structure of the ITR, in particular the 3′ITR.

According to some embodiments, the double-stranded ceDNA molecule comprises at least one uridine-, inosine-, xanthosine-, and/or oxanosine-containing residue. According to further embodiments, the endonuclease has enzymatic activity on the uridine-, inosine-, xanthosine-, and/or oxanosine-containing residue.

According to some embodiments, the endonuclease having enzymatic activity on uridine-, inosine-, xanthosine-, and/or oxanosine-containing residue can nick the modified DNA at the second phosphodiester bond 3′ to the lesion.

According to some embodiments, the 3′ terminal portion of the double-stranded DNA molecule (starting material) comprises a nickase recognition sequence. In one embodiment, the 3′ terminal portion of the dsDNA molecule comprises the sequence 5′-CCAA-3′. In some embodiments the 3′ terminal portion of the dsDNA molecule comprises any one or more of the sequences shown in Table 2 below. Further, since these are unique sequences after a double-stranded ceDNA with special engineered nick sites has been nicked by a nicking endonuclease as shown in Table 2, resultant ssDNA molecules also comprise any one or more of the sequences shown in Table 2 below in its 3′ terminal fragment.

TABLE 2

| Sequence | Nicking Endonuclease |
|---|---|
| 5′-CCAA-3′ | (Nb.BtsI) (Nb.BsrDI) |
| | (Nt.CviPII) |
| 5′-CCAAGC-3′ | (Nb.BbvCI) |
| 5′-CCAACC-3′ | (Nt.BbvCI) |
| 5′-CCAAGAGTCNNNN-3′ | (Nt.BstNBI) - N can be |
| | A, G, C, or T |
| 5′-CCAAG-3′ | (Nb.Bsml) |
| 5′-CCAAC-3′ | (Nb.BssSI) |
| 5′-CCAAGGATCNNNN-3′ | (Nt.AlwI) |
| 5′-CCAAGTCTCN-3′ | (Nt.BsmAI) |
| 5′-CCAAGCTCTTCN-3′ | (Nt.BspQI) |

### B. Phosphorothioate (PS) Modifications

Following the contacting step with the endonuclease, the double-stranded ceDNA described herein is then processed using an exonuclease to produce the ssDNA described herein.

According to some embodiments, the exonuclease is capable removing the nicked strand of the dsDNA construct beginning at the one or more nick sites and ending at the one or more phosphorothioate-modified nucleotides. The exonuclease can be selected from, but is not limited to T7 exonuclease, Lambda exonuclease, T5 exonuclease, and Exonuclease V. According to some embodiments, the exonuclease is T7 exonuclease.

According to some embodiments, the double-stranded closed-ended DNA intermediate comprises phosphorothioate (PS) bonds. The PS bond substitutes a sulfur atom for a non-bridging oxygen in the phosphate backbone of an oligonucleotide. Advantageously, this modification renders the internucleotide linkage resistant to nuclease degradation, and provides accuracy for targeting of the exonuclease. More specifically, this modification is advantageously located in the ITR region in a space where the exonuclease is active, and functions as a lock on the 5′ and/or 3′ ends, rendering the internucleotide linkage resistant to nuclease degradation, and ensuring the accuracy of exonuclease activity.

According to some embodiments, in the methods of production of the single-stranded DNA (ss DNA) molecule, the PS bond substitutes a sulfur atom for a non-bridging oxygen in the phosphate backbone of an oligonucleotide. Advantageously, this modification stabilizes the nucleic acids and renders the internucleotide linkage resistant to nuclease degradation.

According to some embodiments, the one or more phosphorothioate-modified nucleotides of the ss DNA molecule are each independently located in any region selected from A, A′, B, B′, C, C′, D(+) and D(−) of at least one of the first and the optional second ITRs. According to some embodiments, the one or more phosphorothioate-modified nucleotides of the dsDNA construct are each independently located in any region selected from A, A′, B, B′, C, C′, D, and D′ of at least one of the first and the optional second ITRs.

According to some embodiments, the one or more phosphorothioate-modified nucleotides of the ssDNA molecule are each independently located in any region selected from A, A′, and D of at least one of the first and the optional second ITRs. According to some embodiments, the one or more phosphorothioate-modified nucleotides of the dsDNA construct are each independently located in any region selected from A, A′, and D of at least one of the first and the optional second ITRs.

According to some embodiments, the one or more phosphorothioate-modified nucleotides of the ssDNA molecule are each independently located in any region selected from A and A′ of at least one of the first and the optional second ITRs. According to some embodiments, the one or more phosphorothioate-modified nucleotides of the ds DNA construct are each independently located in any region selected from A and A′ of at least one of the first and the optional second ITRs.

According to some embodiments, all of the one or more phosphorothioate-modified nucleotides of the ssDNA molecule in the first ITR are located in A′ region and/or D region of the first ITR. According to some embodiments, all of the one or more phosphorothioate-modified nucleotides of the dsDNA construct in the first ITR are located in A′ region and/or D region of the first ITR.

According to some embodiments, all of the one or more phosphorothioate-modified nucleotides in the first ITR of the ssDNA molecule are located in A region of the first ITR. According to some embodiments, all of the one or more phosphorothioate-modified nucleotides in the first ITR of the dsDNA construct are located in A region of the first ITR.

According to some embodiments, all of the one or more phosphorothioate-modified nucleotides in the second ITR of the ssDNA molecule, if present, are located in A′ region and/or D region of the second ITR. According to some embodiments, all of the one or more phosphorothioate-modified nucleotides in the second ITR of the dsDNA construct, if present, are located in A′ region and/or D region of the second ITR.

According to some embodiments, all of the one or more phosphorothioate-modified nucleotides in the second ITR of the ssDNA molecule, if present, are located in A region of the second ITR. According to some embodiments, all of the one or more phosphorothioate-modified nucleotides dsDNA construct in the second ITR, if present, are located in A region of the second ITR.

According to some embodiments, the one or more phosphorothioate-modified nucleotides of the ssDNA molecule are adjacent to one another. According to some embodiments, the one or more phosphorothioate-modified nucleotides dsDNA construct are adjacent to one another.

According to some embodiments, the one or more phosphorothioate-modified nucleotides of the ssDNA molecule are about 1 to 15 nucleotides from B-B′ arm and C-C′ arm, if present, of the first ITR or the optional second ITR. According to some embodiments, the one or more phosphorothioate-modified nucleotides dsDNA construct are about 1 to 15 nucleotides from B-B′ arm and C-C′ arm, if present, of the first ITR or the optional second ITR.

According to some embodiments, the one or more phosphorothioate-modified nucleotides of the ssDNA molecule are about 1 to 10 nucleotides from B-B′ arm and C-C′ arm, if present, of the first ITR or the optional second ITR. According to some embodiments, the one or more phosphorothioate-modified nucleotides dsDNA construct are about 1 to 10 nucleotides from B-B′ arm and C-C′ arm, if present, of the first ITR or the optional second ITR.

According to some embodiments, the one or more phosphorothioate-modified nucleotides of the ssDNA molecule are about 1 to 5 nucleotides from B-B′ arm and C-C′ arm, if present, of the first ITR or the optional second ITR. According to some embodiments, the one or more phosphorothioate-modified nucleotides dsDNA construct are about 1 to 5 nucleotides from B-B′ arm and C-C′ arm, if present, of the first ITR or the optional second ITR.

According to some embodiments, the one or more phosphorothioate-modified nucleotides of the ssDNA molecule are resistant to exonuclease degradation. According to some embodiments, the one or more phosphorothioate-modified nucleotides containing dsDNA construct are resistant to exonuclease degradation at the PS bonded sequence.

According to some embodiments, at least one of the first and the optional second ITRs of the ssDNA molecule each comprises about 1 to about 60 phosphorothioate-modified nucleotides, e.g., about 1 to about 3, about 1 to about 5, about 1 to about 7, about 1 to about 10, about 1 to about 20, about 1 to about 30, about 1 to about 40, about 1 to about 50, about 10 to about 20, about 10 to about 30, about 10 to about 40, about 10 to about 50, about 20 to about 30, about 20 to about 40, about 20 to about 50, about 30 to about 40, about 30 to about 50, about 40 to about 50, about 25 to about 50, about 5 to about 10, about 5 to about 15, about 5 to about 20 about 5 to about 25. According to some embodiments, at least one of the first and the optional second ITRs dsDNA construct each comprises about 1 to about 60 phosphorothioate-modified nucleotides. According to some embodiments, at least one of the first and the optional second ITRs of the ssDNA molecule each comprises about 1 to about 5 phosphorothioate-modified nucleotides. According to some embodiments, at least one of the first and the optional second ITRs of the ssDNA molecule each comprises about 1 to about 10 phosphorothioate-modified nucleotides. According to some embodiments, at least one of the first and the optional second ITRs of the ssDNA molecule each comprises about 1 to about 15 phosphorothioate-modified nucleotides. According to some embodiments, at least one of the first and the optional second ITRs dsDNA construct each comprises about 1 to about 20 phosphorothioate-modified nucleotides. According to some embodiments, at least one of the first and the optional second ITRs of the ssDNA molecule each comprises about 1 to about 25 phosphorothioate-modified nucleotides. According to some embodiments, at least one of the first and the optional second ITRs dsDNA construct each comprises about 1 to about 30 phosphorothioate-modified nucleotides.

According to some embodiments, the one or more phosphorothioate-modified nucleotides are located at the 5′ end of the ssDNA molecule. According to some embodiments, the one or more phosphorothioate-modified nucleotides are located at the 3′ end of the ssDNA molecule. According to some embodiments, the one or more phosphorothioate-modified nucleotides are located at the 3′ end of the ssDNA molecule, the 5′ end of the ssDNA molecule, or both.

According to some embodiments, the one or more phosphorothioate-modified nucleotides are located upstream of each of the one or more nicking endonuclease recognition sequences. According to some embodiments, the one or more phosphorothioate-modified nucleotides are located at the 5′ end of the first ITR and/or the optional second ITR.

According to some embodiments, the ssDNA molecule comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more phosphorothioate-modified nucleotides. According to some embodiments, wherein the ssDNA molecule comprises at least 1, 2, 3, 4, 5, or more phosphorothioate-modified nucleotides at the 3′ end of the ssDNA molecule, the 5′ end of the ssDNA molecule, or both. According to some embodiments, the ssDNA molecule comprises at least 1, 2, 3, 4, 5 or more phosphorothioate-modified nucleotides upstream of each of the one or more nicking endonuclease recognition sequences. According to some embodiments, the ssDNA molecule comprises at least 1, 2, 3, 4, 5 or more phosphorothioate-modified nucleotides at the 5′ end of the first ITR and/or at least 1, 2, 3, 4, 5 or more phosphorothioate-modified nucleotides at the 5′ end of the optional second ITR.

According to some embodiments, at least one of the first and optional second ITRs of the ssDNA molecule each comprises no more than about 6 phosphorothioate-modified nucleotides. According to some embodiments, at least one of the first and optional second ITRs of the ssDNA molecule each comprises no more than about 5 phosphorothioate-modified nucleotides. According to some embodiments, at least one of the first and optional second ITRs of the ssDNA molecule each comprises no more than about 4 phosphorothioate-modified nucleotides. According to some embodiments, at least one of the first and optional second ITRs of the ssDNA molecule each comprises no more than about 3 phosphorothioate-modified nucleotides. According to some embodiments, at least one of the first and optional second ITRs of the ssDNA molecule each comprises no more than about 2 phosphorothioate-modified nucleotides. According to some embodiments, at least one of the first and optional second ITRs of the ssDNA molecule each comprises no more than about 1 phosphorothioate-modified nucleotides. According to some embodiments, at least one of the first and optional second ITRs dsDNA construct each comprises no more than about 6 phosphorothioate-modified nucleotides. According to some embodiments, at least one of the first and optional second ITRs dsDNA construct each comprises no more than about 5 phosphorothioate-modified nucleotides. According to some embodiments, at least one of the first and optional second ITRs dsDNA construct each comprises no more than about 4 phosphorothioate-modified nucleotides. According to some embodiments, at least one of the first and optional second ITRs dsDNA construct each comprises no more than about 3 phosphorothioate-modified nucleotides. According to some embodiments, at least one of the first and optional second ITRs dsDNA construct each comprises no more than about 2 phosphorothioate-modified nucleotides. According to some embodiments, at least one of the first and optional second ITRs dsDNA construct each comprises no more than about 1 phosphorothioate-modified nucleotides.

According to some embodiments, at least one of the first and optional second ITRs of the ssDNA molecule each comprises about 3, about 4, or about 5 phosphorothioate-modified nucleotides. According to some embodiments, at least one of the first and optional second ITRs dsDNA construct each comprises about 3, about 4, or about 5 phosphorothioate-modified nucleotides.

### C. Transgenes

The ceDNA may comprise a transgene (a nucleic acid sequence of interest) and one or more regulatory sequences that allows and/or controls the expression of the transgene, e.g., an expression cassette. In one embodiment, the expression cassette can comprise one or more of, in this order: an enhancer/promoter, an ORF reporter (transgene), a post-transcription regulatory element (e.g., WPRE), and a polyadenylation and termination signal (e.g., BGH polyA). The expression cassette can also comprise an internal ribosome entry site (IRES) and/or a 2A element. The cis-regulatory elements include, but are not limited to, a promoter, a riboswitch, an insulator, a mir-regulatable element, a post-transcriptional regulatory element, a tissue- and cell type-specific promoter and an enhancer. In some embodiments the ITR can act as the promoter for the transgene. In some embodiments, the ssDNA molecule described herein comprises additional components to regulate expression of the transgene or nucleic acid sequence of interest, for example, a regulatory switch, which are described herein in the section entitled “Regulatory Switches” for controlling and regulating the expression of the transgene, and can include if desired, a regulatory switch which is a kill switch to enable controlled cell death of a cell comprising a ssDNA molecule.

The expression cassette or the nucleic acid sequence of interest in the ssDNA construct can comprise more than 4000 nucleotides, 5000 nucleotides, 10,000 nucleotides or 20,000 nucleotides, or 30,000 nucleotides, or 40,000 nucleotides or 50,000 nucleotides, or any range between about 4000-10,000 nucleotides or 10,000-50,000 nucleotides, or more than 50,000 nucleotides. In some embodiments, the expression cassette can comprise a transgene in the range of 500 to 50,000 nucleotides in length. In some embodiments, the expression cassette can comprise a transgene in the range of 500 to 75,000 nucleotides in length. In some embodiments, the expression cassette can comprise a transgene which is in the range of 500 to 10,000 nucleotides in length. In some embodiments, the expression cassette can comprise a transgene which is in the range of 1000 to 10,000 nucleotides in length. In some embodiments, the expression cassette can comprise a transgene which is in the range of 500 to 5,000 nucleotides in length. The ssDNA molecules described herein do not have the size limitations of encapsidated AAV vectors, thus enable delivery of a large-size expression cassette to provide efficient transgene. In some embodiments, the ssDNA molecules described herein are modified to minimize prokaryote-specific methylation.

An expression cassette can include, for example, an expressible exogenous sequence (e.g., open reading frame) or transgene or nucleic acid sequence of interest that encodes a protein that is either absent, inactive, or insufficient activity in the recipient subject or a gene that encodes a protein having a desired biological or a therapeutic effect. The transgene or nucleic acid sequence of interest can encode a gene product that can function to correct the expression of a defective gene or transcript. In principle, the expression cassette can include any gene that encodes a protein, polypeptide or RNA that is either reduced or absent due to a mutation or which conveys a therapeutic benefit when overexpressed is considered to be within the scope of the disclosure.

The expression cassette can comprise any transgene or nucleic acid sequence of interest useful for treating a disease or disorder in a subject. A ssDNA molecule described herein produced using the synthetic processes as described herein can be used to deliver and express any gene of interest in the subject, which includes but are not limited to, nucleic acids encoding polypeptides, or non-coding nucleic acids (e.g., RNAi, miRs etc.), as well as exogenous genes and nucleotide sequences, including virus sequences in a subjects' genome, e.g., HIV virus sequences and the like. In some embodiments, ssDNA molecules described herein are used for therapeutic purposes (e.g., for medical, diagnostic, or veterinary uses). In certain embodiments, ssDNA molecules described herein are useful to express any gene of interest in the subject, which includes one or more polypeptides, peptides, ribozymes, peptide nucleic acids, siRNAs, RNAis, antisense oligonucleotides, antisense polynucleotides, or RNAs (coding or non-coding; e.g., siRNAs, shRNAs, micro-RNAs, mRNA or gRNA, and their antisense counterparts (e.g., antagoMiR)), antibodies, antigen binding fragments, or any combination thereof.

The expression cassette can also encode polypeptides, sense or antisense oligonucleotides, or RNAs (coding or non-coding; e.g., siRNAs, shRNAs, micro-RNAs, and their antisense counterparts (e.g., antagoMiR)). Expression cassettes can include an exogenous sequence that encodes a reporter protein to be used for experimental or diagnostic purposes, such as β-lactamase, β-galactosidase (LacZ), alkaline phosphatase, thymidine kinase, green fluorescent protein (GFP), chloramphenicol acetyltransferase (CAT), luciferase, and others well known in the art.

Sequences provided in the expression cassette, expression construct of ssDNA molecules described herein can be codon optimized for the target host cell. As used herein, the term “codon optimized” or “codon optimization” refers to the process of modifying a nucleic acid sequence for enhanced expression in the cells of the vertebrate of interest, e.g., mouse or human, by replacing at least one, more than one, or a significant number of codons of the native sequence (e.g., a prokaryotic sequence) with codons that are more frequently or most frequently used in the genes of that vertebrate. Various species exhibit particular bias for certain codons of a particular amino acid. Typically, codon optimization does not alter the amino acid sequence of the original translated protein. Optimized codons can be determined using e.g., Aptagen's GENEFORGE® codon optimization and custom gene synthesis platform (Aptagen, Inc., 2190 Fox Mill Rd. Suite 300, Herndon, Va. 20171) or another publicly available database.

In some embodiments, a transgene or nucleic acid sequence of interest expressed by the ssDNA molecules is a therapeutic gene. In some embodiments, a therapeutic gene is an antibody, or antibody fragment, or antigen-binding fragment thereof, e.g., a neutralizing antibody or antibody fragment and the like.

In particular, a therapeutic gene is one or more therapeutic agent(s), including, but not limited to, for example, protein(s), polypeptide(s), peptide(s), enzyme(s), antibodies, antigen binding fragments, as well as variants, and/or active fragments thereof, for use in the treatment, prophylaxis, and/or amelioration of one or more symptoms of a disease, dysfunction, injury, and/or disorder. Exemplary therapeutic genes are described herein in the section entitled “Method of Treatment”.

There are many structural features of ssDNA molecules described herein that differ from plasmid-based expression vectors. ssDNA molecules produced by the synthetic methods herein may possess one or more of the following features: the lack of original (i.e. not inserted) bacterial DNA, the lack of a prokaryotic origin of replication, being self-containing, i.e., they do not require any sequences other than the two ITRs, including the Rep binding and terminal resolution sites (RBS and TRS), and an exogenous sequence between the ITRs, the presence of ITR sequences that form hairpins, and the absence of bacterial-type DNA methylation or indeed any other methylation associated with production in a given cell type and considered abnormal by a mammalian host. In general, it is preferred for the present vectors not to contain any prokaryotic DNA but it is contemplated that some prokaryotic DNA may be inserted as an exogenous sequence, as a non-limiting example in a promoter or enhancer region.

There are several advantages of using ssDNA molecules described herein over plasmid-based expression vectors. Such advantages include, but are not limited to: 1) plasmids contain bacterial DNA sequences and are subjected to prokaryotic-specific methylation, e.g., 6-methyl adenosine and 5-methyl cytosine methylation, whereas capsid-free AAV vector sequences are of eukaryotic origin and do not undergo prokaryotic-specific methylation; as a result, capsid-free AAV vectors are less likely to induce inflammatory and immune responses compared to plasmids; 2) while plasmids require the presence of a resistance gene during the production process, ssDNA molecules of the present disclosure do not; 3) while a circular plasmid is not delivered to the nucleus upon introduction into a cell and requires overloading to bypass degradation by cellular nucleases, ssDNA molecules contain viral cis-elements, i.e., ITRs, that confer resistance to nucleases and can be designed to be targeted and delivered to the nucleus. It is hypothesized that the minimal defining elements indispensable for ITR function are a Rep-binding site (RBS; 5′-GCGCGCTCGCTCGCTC-3′ for AAV2) and a terminal resolution site (TRS; 5′-AGTTGG-3′ for AAV2) plus a variable palindromic sequence allowing for hairpin formation; and 4) ssDNA molecules do not have the over-representation of CpG dinucleotides often found in prokaryote-derived plasmids that reportedly binds a member of the Toll-like family of receptors, eliciting a T cell-mediated immune response.

### D. Inverted Terminal Repeats (ITRs)

As set forth herein, according to some aspects, the disclosure provides a ceDNA molecule comprising at least one nucleic acid sequence of interest flanked by at least one stem-loop structure comprising a partial DNA duplex and at least one loop on the 3′ end. In some embodiments, the ceDNA molecule comprises at least one stem-loop structure comprising a partial DNA duplex and at least one loop at the 5′ end.

According to some aspects, ceDNA molecules contain a transgene or heterologous nucleic acid sequence positioned between two inverted terminal repeat (ITR) sequences, where the ITR sequences can be an asymmetrical ITR pair or a symmetrical- or substantially symmetrical ITR pair, as these terms are defined herein. A ceDNA molecule and dsDNA construct as disclosed herein can comprise ITR sequences that are selected from any of: (i) at least one WT ITR and at least one modified AAV inverted terminal repeat (mod-ITR) (e.g., asymmetric modified ITRs); (ii) two modified ITRs where the mod-ITR pair have a different three-dimensional spatial organization with respect to each other (e.g., asymmetric modified ITRs), or (iii) symmetrical or substantially symmetrical WT-WT ITR pair, where each WT-ITR has the same three-dimensional spatial organization, or (iv) symmetrical or substantially symmetrical modified ITR pair, where each mod-ITR has the same three-dimensional spatial organization, where the methods of the present disclosure may further include a delivery system, such as but not limited to a liposome nanoparticle delivery system.

In some embodiments, the ITR sequence can be from viruses of the Parvoviridae family, which includes two subfamilies: Parvovirinae, which infect vertebrates, and Densovirinae, which infect insects. The subfamily Parvovirinae (referred to as the parvoviruses) includes the genus Dependovirus, the members of which, under most conditions, require coinfection with a helper virus such as adenovirus or herpes virus for productive infection. The genus Dependovirus includes adeno-associated virus (AAV), which normally infects humans (e.g., serotypes 2, 3A, 3B, 5, and 6) or primates (e.g., serotypes 1 and 4), and related viruses that infect other warm-blooded animals (e.g., bovine, canine, equine, and ovine adeno-associated viruses). The parvoviruses and other members of the Parvoviridae family are generally described in Kenneth I. Berns, “Parvoviridae: The Viruses and Their Replication,” Chapter 69 in FIELDS VIROLOGY (3d Ed. 1996).

While ITRs exemplified in the specification and Examples herein are AAV2 WT-ITRs, one of ordinary skill in the art is aware that one can as stated above use ITRs from any known parvovirus, for example a dependovirus such as AAV (e.g., AAV1, AAV2, AAV3, AAV4, AAV5, AAV 5, AAV7, AAV8, AAV9, AAV10, AAV 11, AAV12, AAVrh8, AAVrh10, AAV-DJ, and AAV-DJ8 genome. E.g., NCBI: NC 002077; NC 001401; NC001729; NC001829; NC006152; NC 006260; NC 006261), chimeric ITRs, or ITRs from any synthetic AAV. In some embodiments, the AAV can infect warm-blooded animals, e.g., avian (AAAV), bovine (BAAV), canine, equine, and ovine adeno-associated viruses. In some embodiments the ITR is from B19 parvovirus (GenBank Accession No: NC 000883), Minute Virus from Mouse (MVM) (GenBank Accession No. NC 001510); goose parvovirus (GenBank Accession No. NC 001701); snake parvovirus 1 (GenBank Accession No. NC 006148). In some embodiments, the 5′ WT-ITR can be from one serotype and the 3′ WT-ITR from a different serotype, as discussed herein.

An ordinarily skilled artisan is aware that ITR sequences have a common structure of a double-stranded Holliday junction, which typically is a T-shaped or Y-shaped hairpin structure, where each WT-ITR is formed by two palindromic arms or loops (B-B′ and C-C′) embedded in a larger palindromic arm (A-A′), and a single-stranded D sequence, (where the order of these palindromic sequences defines the flip or flop orientation of the ITR). See, for example, structural analysis and sequence comparison of ITRs from different AAV serotypes (AAV1-AAV6) and described in Grimm et al., J. Virology, 2006; 80 (1); 426-439; Yan et al., J. Virology, 2005; 364-379; Duan et al., Virology 1999; 261; 8-14. One of ordinary skill in the art can readily determine WT-ITR sequences from any AAV serotype for use in a ssDNA molecules and dsDNA constructs based on the exemplary AAV2 ITR sequences provided herein. See, for example, the sequence comparison of ITRs from different AAV serotypes (AAV1-AAV6, and avian AAV (AAAV) and bovine AAV (BAAV)) described in Grimm et al., J. Virology, 2006; 80 (1); 426-439; that show the % identity of the 3′ ITR of AAV2 to the 3′ ITR from other serotypes: AAV-1 (84%), AAV-3 (86%), AAV-4 (79%), AAV-5 (58%), AAV-6 (3′ITR) (100%) and AAV-6 (3′ITR) (82%).

According to some other embodiments, at least one of the first ITR and the optional second ITR oligonucleotides comprising one or more phosphorothioate-modified nucleotides of the present invention can further comprise one or more functional moieties. In one embodiment, the at least one function moiety is an aptamer sequence, optionally wherein the aptamer sequence has a high binding affinity to a nuclear localized protein. In another embodiment, the at least one function moiety is a nuclear localization peptide conjugated to the at least one of the ITR oligonucleotides. In another embodiment, the at least one function moiety is a fluorophore chemically conjugated to the ITR oligonucleotides.

### E. Regulatory Elements

The single-stranded DNA (ssDNA) molecules as described herein can further comprise a specific combination of cis-regulatory elements. The cis-regulatory elements include, but are not limited to, a promoter, a riboswitch, an insulator, a miR-regulatable element, a post-transcriptional regulatory element, a tissue- and cell type-specific promoter and an enhancer. In some embodiments, the single-stranded DNA (ssDNA) molecule described herein comprises additional components to regulate expression of the transgene or nucleic acid of interest, for example, regulatory switches as described herein, to regulate the expression of the transgene or nucleic acid of interest, or a kill switch, which can kill a cell comprising the single-stranded DNA (ssDNA) molecule described herein. Regulatory elements, including Regulatory Switches that can be used in the present disclosure are more fully discussed in International application PCT/US18/49996 (published as International patent publication No. WO 2019/051255 A1), which is incorporated herein in its entirety by reference.

According to some embodiments, the second nucleotide sequence includes a regulatory sequence, and a nucleotide sequence encoding a nuclease. In certain embodiments the gene regulatory sequence is operably linked to the nucleotide sequence encoding the nuclease. In certain embodiments, the regulatory sequence is suitable for controlling the expression of the nuclease in a host cell. In certain embodiments, the regulatory sequence includes a suitable promoter sequence, being able to direct transcription of a gene operably linked to the promoter sequence, such as a nucleotide sequence encoding the nuclease(s) of the present disclosure. In certain embodiments, the second nucleotide sequence includes an intron sequence linked to the 5′ terminus of the nucleotide sequence encoding the nuclease. In certain embodiments, an enhancer sequence is provided upstream of the promoter to increase the efficacy of the promoter. In certain embodiments, the regulatory sequence includes an enhancer and a promoter, wherein the second nucleotide sequence includes an intron sequence upstream of the nucleotide sequence encoding a nuclease, wherein the intron includes one or more nuclease cleavage site(s), and wherein the promoter is operably linked to the nucleotide sequence encoding the nuclease.

The single-stranded DNA (ssDNA) molecules described herein and dsDNA molecules produced using the synthetic process as described herein can further comprise a specific combination of cis-regulatory elements such as WHP posttranscriptional regulatory element (WPRE) and BGH polyA. Suitable expression cassettes for use in expression constructs are not limited by the packaging constraint imposed by the viral capsid.

### (i) Promoters

It will be appreciated by one of ordinary skill in the art that promoters used in the synthetically produced single-stranded DNA (ssDNA) molecules described herein and dsDNA molecules of the disclosure should be tailored as appropriate for the specific sequences they are promoting. For example, a guide RNA may not require a promoter at all, since its function is to form a duplex with a specific target sequence on the native DNA to effect a recombination event. In contrast, a nuclease encoded by the ssDNA molecule or the dsDNA construct vector would benefit from a promoter so that it can be efficiently expressed from the vector—and, optionally, in a regulatable fashion.

Expression cassettes of the present disclosure include a promoter, which can influence overall expression levels as well as cell-specificity. For transgene expression, they can include a highly active virus-derived immediate early promoter. Expression cassettes can contain tissue-specific eukaryotic promoters to limit transgene expression to specific cell types and reduce toxic effects and immune responses resulting from unregulated, ectopic expression. In preferred embodiments, an expression cassette can contain a synthetic regulatory element, such as a CAG promoter. The CAG promoter comprises (i) the cytomegalovirus (CMV) early enhancer element, (ii) the promoter, the first exon and the first intron of chicken beta-actin gene, and (iii) the splice acceptor of the rabbit beta-globin gene. Alternatively, an expression cassette can contain an Alpha-1-antitrypsin (AAT) promoter, a liver specific (LP1) promoter, a Human elongation factor-1 alpha (EF1a) promoter, or a human transthyretin (TTR) promoter. In some embodiments, the expression cassette includes one or more constitutive promoters, for example, a retroviral Rous sarcoma virus (RSV) LTR promoter (optionally with the RSV enhancer), or a cytomegalovirus (CMV) immediate early promoter (optionally with the CMV enhancer). Alternatively, an inducible promoter, a native promoter for a transgene, a tissue-specific promoter, or various promoters known in the art can be used.

Suitable promoters, including those described above, can be derived from viruses and can therefore be referred to as viral promoters, or they can be derived from any organism, including prokaryotic or eukaryotic organisms. Suitable promoters can be used to drive expression by any RNA polymerase (e.g., pol I, pol II, pol III). Exemplary promoters include, but are not limited to the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6) (Miyagishi et al., Nature Biotechnology 20, 497-500 (2002)), an enhanced U6 promoter (e.g., Xia et al., Nucleic Acids Res. 2003 Sep. 1; 31 (17)), a human H1 promoter (H1), a CAG promoter, a human alpha 1-antitypsin (HAAT) promoter, and the like. In certain embodiments, these promoters are altered at their downstream intron containing end to include one or more nuclease cleavage sites. In certain embodiments, the DNA containing the nuclease cleavage site(s) is foreign to the promoter DNA.

In one embodiment, the promoter used is the native promoter of the gene encoding the therapeutic protein. The promoters and other regulatory sequences for the respective genes encoding the therapeutic proteins are known and have been characterized. The promoter region used may further include one or more additional regulatory sequences (e.g., native enhancers). It is preferred that a gap is located 5′ upstream of a promoter.

### (ii) Polyadenylation Sequences

A sequence encoding a polyadenylation sequence can be included in the synthetically produced vector to stabilize an mRNA expressed from the single-stranded DNA (ssDNA) molecules (e.g., a synthetic vector, e.g., a single-stranded (ss) synthetic vector), and to aid in nuclear export and translation. In one embodiment, the synthetically produced vector does not include a polyadenylation sequence. In other embodiments, the vector includes at least 1, at least 2, at least 3, at least 4, at least 5, at least 10, at least 15, at least 20, at least 25, at least 30, at least 40, least 45, at least 50 or more adenine dinucleotides. In some embodiments, the polyadenylation sequence comprises about 43 nucleotides, about 40-50 nucleotides, about 40-55 nucleotides, about 45-50 nucleotides, about 35-50 nucleotides, or any range there between.

The expression cassettes can include a poly-adenylation sequence known in the art or a variation thereof, such as a naturally occurring sequence isolated from bovine BGHpA or a virus SV40pA, or a synthetic sequence. Some expression cassettes can also include SV40 late polyA signal upstream enhancer (USE) sequence. In some embodiments, the, USE can be used in combination with SV40pA or heterologous poly-A signal.

The expression cassettes can also include a post-transcriptional element to increase the expression of a transgene. In some embodiments, Woodchuck Hepatitis Virus (WHP) posttranscriptional regulatory element (WPRE) is used to increase the expression of a transgene. Other posttranscriptional processing elements such as the post-transcriptional element from the thymidine kinase gene of herpes simplex virus, or hepatitis B virus (HBV) can be used. Secretory sequences can be linked to the transgenes, e.g., VH-02 and VK-A26 sequences.

(iii) Nuclear Localization Sequences

In some embodiments, the vector encoding an RNA guided endonuclease comprises one or more nuclear localization sequences (NLSs), for example, 1, 2, 3, 4, 5, 6, 7, 8, 9, 10, or more NLSs. In some embodiments, the one or more NLSs are located at or near the amino-terminus, at or near the carboxy-terminus, or a combination of these (e.g., one or more NLS at the amino-terminus and/or one or more NLS at the carboxy terminus). When more than one NLS is present, each can be selected independently of the others, such that a single NLS is present in more than one copy and/or in combination with one or more other NLSs present in one or more copies. Non-limiting examples of NLSs are shown in Table 3 below.

TABLE 3
Exemplary Nuclear Localization Sequences (NLS)

| SOURCE | SEQUENCE |
|---|---|
| SV40 virus large T- | PKKKRKV |
| antigen | |
| nucleoplasmin | KRPAATKKAGQAKKKK |
| c-myc | PAAKRVKLD |
| | RQRRNELKRSP |
| hRNPA1 M9 | NQSSNFGPMKGGNFGGRSSGP |
| | YGGGGQYFAKPRNQGGY |
| IBB domain from | RMRIZFKNKGKDTAELRRRRV |
| importin-alpha | EVSVELRKAKKDEQILKRRNV |
| myoma T protein | VSRKRPRP |
| | PPKKARED |
| human p53 | PQPKKKPL |
| mouse c-abl IV | SALIKKKKKMAP |
| influenza virus NS1 | DRLRR |
| | PKQKKRK |
| Hepatitis virus delta | RKLKKKIKKL |
| antigen | |
| mouse Mx1 protein | REKKKFLKRR |
| human poly(ADP- | KRKGDEVDGVDEVAKKKSKK |
| ribose) polymerase | |
| steroid hormone | RKCLQAGMNLEARKTKK |
| receptors (human) | |
| glucocorticoid | |

### F. Additional Components

The single-stranded DNA (ssDNA) molecules described herein and dsDNA molecules produced using the synthetic process as described herein may contain nucleotides that encode other components for gene expression. For example, to select for specific gene targeting events, a protective shRNA may be embedded in a microRNA and inserted into a recombinant single-stranded DNA (ssDNA) molecule described herein designed to integrate site-specifically into the highly active locus, such as an albumin locus. Such embodiments may provide a system for in vivo selection and expansion of gene-modified hepatocytes in any genetic background such as described in Nygaard et al., A universal system to select gene-modified hepatocytes in vivo, Gene Therapy, Jun. 8, 2016. The single-stranded DNA (ssDNA) molecules described herein of the present disclosure may contain one or more selectable markers that permit selection of transformed, transfected, transduced, or the like cells. A selectable marker is a gene the product of which provides for biocide or viral resistance, resistance to heavy metals, prototrophy to auxotrophs, NeoR, and the like. In certain embodiments, positive selection markers are incorporated into the donor sequences such as NeoR. Negative selections markers may be incorporated downstream the donor sequences, for example a nucleic acid sequence HSV-tk encoding a negative selection marker may be incorporated into a nucleic acid construct downstream the donor sequence.

In embodiments, the single-stranded DNA (ssDNA) molecules described herein and dsDNA molecules produced using the synthetic process as described herein can be used for gene editing, for example, as disclosed in International Application PCT/US2018/064242, filed on Dec. 6, 2018 (published as International patent publication No. WO 2019/113310 A1), which is incorporated herein in its entirety by reference, and may include one or more of: a 5′ homology arm, a 3′ homology arm, a polyadenylation site upstream and proximate to the “homology arm. Exemplary homology arms are 5′ and 3′ albumin homology arms or CCR5 5′- and 3′ homology arms.

### G. Switches

A molecular regulatory switch is one which generates a measurable change in state in response to a signal. Such regulatory switches can be usefully combined with the single-stranded DNA (ssDNA) molecules described herein and dsDNA molecules produced using the synthetic process as described herein to control the output of expression of the transgene from the single-stranded DNA (ssDNA) molecules described herein. In some embodiments, the single-stranded DNA (ssDNA) molecule described herein comprises a regulatory switch that serves to fine tune expression of the transgene. For example, it can serve as a biocontainment function of the single-stranded DNA (ssDNA) molecule described herein. In some embodiments, the switch is an “ON/OFF” switch that is designed to start or stop (i.e., shut down) expression of the gene of interest in the synthetic AAV in a controllable and regulatable fashion. In some embodiments, the switch can include a “kill switch” that can instruct the cell comprising the single-stranded DNA (ssDNA) molecule described herein to undergo cell programmed death once the switch is activated. Exemplary regulatory switches encompassed for use in a single-stranded DNA (ssDNA) molecule described herein can be used to regulate the expression of a transgene, and are more fully discussed in International application PCT/US18/49996 (published as International patent publication No. WO 2019/051255 A1), which is incorporated herein in its entirety by reference.

### (i) Binary Regulatory Switches

In some embodiments, the single-stranded DNA (ssDNA) molecule described herein produced using the synthetic process as described herein comprises a regulatory switch that can serve to controllably modulate expression of the transgene. For example, the expression cassette located between the ITRs of the single-stranded DNA (ssDNA) molecule described herein may additionally comprise a regulatory region, e.g., a promoter, cis-element, repressor, enhancer etc., that is operatively linked to the gene of interest, where the regulatory region is regulated by one or more cofactors or exogenous agents. By way of example only, regulatory regions can be modulated by small molecule switches or inducible or repressible promoters. Non-limiting examples of inducible promoters are hormone-inducible or metal-inducible promoters. Other exemplary inducible promoters/enhancer elements include, but are not limited to, an RU486-inducible promoter, an ecdysone-inducible promoter, a rapamycin-inducible promoter, and a metallothionein promoter.

### (ii) Small Molecule Regulatory Switches

A variety of art-known small-molecule based regulatory switches are known in the art and can be combined with the synthetically produced single-stranded DNA (ssDNA) molecule described herein disclosed herein to form a regulatory-switch controlled single-stranded DNA (ssDNA) molecule described herein. In some embodiments, the regulatory switch can be selected from any one or a combination of: an orthogonal ligand/nuclear receptor pair, for example retinoid receptor variant/LG335 and GRQCIMFI, along with an artificial promoter controlling expression of the operatively linked transgene, such as that as disclosed in Taylor, et al. BMC Biotechnology 10 (2010): 15; engineered steroid receptors, e.g., modified progesterone receptor with a C-terminal truncation that cannot bind progesterone but binds RU486 (mifepristone) (U.S. Pat. No. 5,364,791); an ecdysone receptor from Drosophila and their ecdysteroid ligands (Saez, et al., PNAS, 97 (26) (2000), 14512-14517; or a switch controlled by the antibiotic trimethoprim (TMP), as disclosed in Sando R 3^{rd}; Nat Methods. 2013, 10 (11): 1085-8. In some embodiments, the regulatory switch to control the transgene or expressed by the single-stranded DNA (ssDNA) molecule (e.g., a synthetic vector, e.g., a single-stranded (ss) synthetic vector) is a pro-drug activation switch, such as that disclosed in U.S. Pat. Nos. 8,771,679, and 6,339,070.

(iii) “Passcode” Regulatory Switches

In some embodiments the regulatory switch can be a “passcode switch” or “passcode circuit”. Passcode switches allow fine tuning of the control of the expression of the transgene from the synthetically produced single-stranded DNA (ssDNA) molecule described herein when specific conditions occur—that is, a combination of conditions need to be present for transgene expression and/or repression to occur. For example, for expression of a transgene to occur at least conditions A and B must occur. A passcode regulatory switch can be any number of conditions, e.g., at least 2, or at least 3, or at least 4, or at least 5, or at least 6 or at least 7 or more conditions to be present for transgene expression to occur. In some embodiments, at least 2 conditions (e.g., A, B conditions) need to occur, and in some embodiments, at least 3 conditions need to occur (e.g., A, B and C, or A, B and D). By way of an example only, for gene expression from a synthetic AAV to occur that has a passcode “ABC” regulatory switch, conditions A, B and C must be present. Conditions A, B and C could be as follows; condition A is the presence of a condition or disease, condition B is a hormonal response, and condition C is a response to the transgene expression. For example, if the transgene edits a defective EPO gene, Condition A is the presence of Chronic Kidney Disease (CKD), Condition B occurs if the subject has hypoxic conditions in the kidney, Condition C is that Erythropoietin-producing cells (EPC) recruitment in the kidney is impaired; or alternatively, HIF-2 activation is impaired. Once the oxygen levels increase or the desired level of EPO is reached, the transgene turns off again until 3 conditions occur, turning it back on.

In some embodiments, a passcode regulatory switch or “Passcode circuit” encompassed for use in the synthetically produced single-stranded DNA (ssDNA) molecule described herein comprises hybrid transcription factors (TFs) to expand the range and complexity of environmental signals used to define biocontainment conditions. As opposed to a deadman switch which triggers cell death in the presence of a predetermined condition, the “passcode circuit” allows cell survival or transgene expression in the presence of a particular “passcode”, and can be easily reprogrammed to allow transgene expression and/or cell survival only when the predetermined environmental condition or passcode is present.

Any and all combinations of regulatory switches disclosed herein, e.g., small molecule switches, nucleic acid-based switches, small molecule-nucleic acid hybrid switches, post-transcriptional transgene regulation switches, post-translational regulation, radiation-controlled switches, hypoxia-mediated switches and other regulatory switches known by persons of ordinary skill in the art as disclosed herein can be used in a passcode regulatory switch as disclosed herein. Regulatory switches encompassed for use are also discussed in the review article Kis et al., J R Soc Interface. 12: 20141000 (2015), and summarized in Table 1 of Kis et al. In some embodiments, a regulatory switch for use in a passcode system can be selected from any or a combination of the switches in Table 4 below.

### (iv) Nucleic Acid-Based Regulatory Switches to Control Transgene Expression

In some embodiments, the regulatory switch to control the transgene expressed by the synthetically produced single-stranded DNA (ssDNA) molecule described herein is based on a nucleic-acid based control mechanism. Exemplary nucleic acid control mechanisms are known in the art and are envisioned for use. For example, such mechanisms include riboswitches, such as those disclosed in, e.g., US2009/0305253, US2008/0269258, US2017/0204477, WO2018026762A1, U.S. Pat. No. 9,222,093 and EP application EP288071, and also disclosed in the review by Villa J K et al., Microbiol Spectr. 2018 May; 6 (3). Also included are metabolite-responsive transcription biosensors, such as those disclosed in WO2018/075486 and WO2017/147585. Other art-known mechanisms envisioned for use include silencing of the transgene with an siRNA or RNAi molecule (e.g., miR, shRNA). For example, the single-stranded DNA (ssDNA) molecule described herein can comprise a regulatory switch that encodes an RNAi molecule that is complementary to the transgene expressed by the single-stranded DNA (ssDNA) molecule described herein. When such RNAi is expressed even if the transgene is expressed by the single-stranded DNA (ssDNA) molecule described herein, it will be silenced by the complementary RNAi molecule, and when the RNAi is not expressed when the transgene is expressed by the single-stranded DNA (ssDNA) molecule described herein the transgene is not silenced by the RNAi.

In some embodiments, the regulatory switch is a tissue-specific self-inactivating regulatory switch, for example as disclosed in US2002/0022018, whereby the regulatory switch deliberately switches transgene expression off at a site where transgene expression might otherwise be disadvantageous. In some embodiments, the regulatory switch is a recombinase reversible gene expression system, for example as disclosed in US2014/0127162 and U.S. Pat. No. 8,324,436.

### (v) Post-Transcriptional and Post-Translational Regulatory Switches.

In some embodiments, the regulatory switch to control the transgene or gene of interest expressed by the synthetically produced single-stranded DNA (ssDNA) molecule described herein is a post-transcriptional modification system. For example, such a regulatory switch can be an aptazyme riboswitch that is sensitive to tetracycline or theophylline, as disclosed in US2018/0119156, GB201107768, WO2001/064956A3, EP U.S. Pat. No. 2,707,487 and Beilstein et al., ACS Synth. Biol., 2015, 4 (5), pp 526-534; Zhong et al., Elife. 2016 Nov. 2; 5. Pii: e18858. In some embodiments, it is envisioned that a person of ordinary skill in the art could encode both the transgene and an inhibitory siRNA which contains a ligand sensitive (OFF-switch) aptamer, the net result being a ligand sensitive ON-switch.

### (vi) Other Exemplary Regulatory Switches

Any known regulatory switch can be used in the synthetically produced ssDNA molecules to control the gene expression of the transgene expressed by the single-stranded DNA (ssDNA) molecule described herein, including those triggered by environmental changes. Additional examples include, but are not limited to; the BOC method of Suzuki et al., Scientific Reports 8; 10051 (2018); genetic code expansion and a non-physiologic amino acid; radiation-controlled or ultra-sound controlled on/off switches (see, e.g., Scott S et al., Gene Ther. 2000 July; 7(13):1121-5; U.S. Pat. Nos. 5,612,318; 5,571,797; 5,770,581; 5,817,636; and WO1999/025385A1. In some embodiments, the regulatory switch is controlled by an implantable system, e.g., as disclosed in U.S. Pat. No. 7,840,263; US2007/0190028A1 where gene expression is controlled by one or more forms of energy, including electromagnetic energy, that activates promoters operatively linked to the transgene in the single-stranded DNA (ssDNA) molecule described herein.

In some embodiments, a regulatory switch envisioned for use in the synthetically produced single-stranded DNA (ssDNA) molecule described herein is a hypoxia-mediated or stress-activated switch, e.g., such as those disclosed in WO1999060142A2, U.S. Pat. Nos. 5,834,306; 6,218,179; 6,709,858; US2015/0322410; Greco et al., (2004) Targeted Cancer Therapies 9, S368, as well as FROG, TOAD and NRSE elements and conditionally inducible silence elements, including hypoxia response elements (HREs), inflammatory response elements (IREs) and shear-stress activated elements (SSAEs), e.g., as disclosed in U.S. Pat. No. 9,394,526. Such an embodiment is useful for turning on expression of the transgene from the single-stranded DNA (ssDNA) molecule described herein after ischemia or in ischemic tissues, and/or tumors.

(vii) Kill Switches

Other embodiments of the disclosure relate to a synthetically produced single-stranded DNA (ssDNA) molecules described herein) and dsDNA molecules comprising a kill switch. A kill switch as disclosed herein enables a cell comprising the single-stranded DNA (ssDNA) molecule described herein to be killed or undergo programmed cell death as a means to permanently remove an introduced single-stranded DNA (ssDNA) molecule described herein from the subject's system. It will be appreciated by one of ordinary skill in the art that use of kill switches in the synthetically produced single-stranded DNA (ssDNA) molecule described herein of the disclosure would be typically coupled with targeting of the single-stranded DNA (ssDNA) molecule described herein to a limited number of cells that the subject can acceptably lose or to a cell type where apoptosis is desirable (e.g., cancer cells). In all aspects, a “kill switch” as disclosed herein is designed to provide rapid and robust cell killing of the cell comprising the single-stranded DNA (ssDNA) molecule described herein in the absence of an input survival signal or other specified condition. Stated another way, a kill switch encoded by a single-stranded DNA (ssDNA) molecule described herein can restrict cell survival of a cell comprising a single-stranded DNA (ssDNA) molecule described herein to an environment defined by specific input signals. Such kill switches serve as a biological biocontainment function should it be desirable to remove the synthetically produced single-stranded DNA (ssDNA) molecule described herein from a subject or to ensure that it will not express the encoded transgene.

Accordingly, kill switches are synthetic biological circuits in the ssDNA molecule or the dsDNA construct that couple environmental signals with conditional survival of the cell comprising the ssDNA molecule or the dsDNA construct. In some embodiments different ssDNA molecules can be designed to have different kill switches.

In some embodiments, a single-stranded DNA (ssDNA) molecule described herein can comprise a kill switch which is a modular biological containment circuit. In some embodiments, a kill switch encompassed for use in the ssDNA molecule or the dsDNA construct is disclosed in WO2017/059245, which describes a switch referred to as a “Deadman kill switch” that comprises a mutually inhibitory arrangement of at least two repressible sequences, such that an environmental signal represses the activity of a second molecule in the construct (e.g., a small molecule-binding transcription factor is used to produce a “survival” state due to repression of toxin production). In cells comprising a single-stranded DNA (ssDNA) molecule described herein comprising a deadman kill switch, upon loss of the environmental signal, the circuit switches permanently to the “death” state, where the toxin is now derepressed, resulting in toxin production which kills the cell. In another embodiment, a synthetic biological circuit referred to as a “Passcode circuit” or “Passcode kill switch” that uses hybrid transcription factors (TFs) to construct complex environmental requirements for cell survival, is provided. The Deadman and Passcode kill switches described in WO2017/059245 are particularly useful for use in single-stranded DNA (ssDNA) molecule described herein, as they are modular and customizable, both in terms of the environmental conditions that control circuit activation and in the output modules that control cell fate. With the proper choice of toxins, including, but not limited to an endonuclease, e.g., a EcoRI, Passcode circuits present in the ssDNA molecule or the dsDNA construct can be used to not only kill the host cell comprising ssDNA molecule or the dsDNA construct but also to degrade its genome and accompanying plasmids.

Other kill switches known to a person of ordinary skill in the art are encompassed for use in the single-stranded DNA (ssDNA) molecule described herein as disclosed herein, e.g., as disclosed in US2010/0175141; US2013/0009799; US2011/0172826; US2013/0109568, as well as kill switches disclosed in Jusiak et al., Reviews in Cell Biology and molecular Medicine; 2014; 1-56; Kobayashi et al., PNAS, 2004; 101; 8419-9; Marchisio et al., Int. Journal of Biochem and Cell Biol., 2011; 43; 310-319; and in Reinshagen et al., Science Translational Medicine, 2018, 11.

Accordingly, in some embodiments, the single-stranded DNA (ssDNA) molecule described herein can comprise a kill switch nucleic acid construct, which comprises the nucleic acid encoding an effector toxin or reporter protein, where the expression of the effector toxin (e.g., a death protein) or reporter protein is controlled by a predetermined condition. For example, a predetermined condition can be the presence of an environmental agent, such as, e.g., an exogenous agent, without which the cell will default to expression of the effector toxin (e.g., a death protein) and be killed. In alternative embodiments, a predetermined condition is the presence of two or more environmental agents, e.g., the cell will only survive when two or more necessary exogenous agents are supplied, and without either of which, the cell comprising the single-stranded DNA (ssDNA) molecule described herein is killed.

In some embodiments, the single-stranded DNA (ssDNA) molecule described herein is modified to incorporate a kill-switch to destroy the cells comprising the single-stranded DNA (ssDNA) molecule described herein to effectively terminate the in vivo expression of the transgene being expressed by the ssDNA molecule or the dsDNA construct (e.g., therapeutic gene, protein or peptide etc.). Specifically, the single-stranded DNA (ssDNA) molecule described herein is further genetically engineered to express a switch-protein that is not functional in mammalian cells under normal physiological conditions. Only upon administration of a drug or environmental condition that specifically targets this switch-protein, the cells expressing the switch-protein will be destroyed thereby terminating the expression of the therapeutic protein or peptide. For instance, it was reported that cells expressing HSV-thymidine kinase can be killed upon administration of drugs, such as ganciclovir and cytosine deaminase. See, for example, Dey and Evans, Suicide Gene Therapy by Herpes Simplex Virus-1 Thymidine Kinase (HSV-TK), in Targets in Gene Therapy, edited by You (2011); and Beltinger et al., Proc. Natl. Acad. Sci. USA 96 (15): 8699-8704 (1999). In some embodiments the ssDNA molecule or the dsDNA construct can comprise a siRNA kill switch referred to as DISE (Death Induced by Survival gene Elimination) (Murmann et al., Oncotarget. 2017; 8:84643-84658. Induction of DISE in ovarian cancer cells in vivo).

In some aspects, a deadman kill switch is a biological circuit or system rendering a cellular response sensitive to a predetermined condition, such as the lack of an agent in the cell growth environment, e.g., an exogenous agent. Such a circuit or system can comprise a nucleic acid construct comprising expression modules that form a deadman regulatory circuit sensitive to the predetermined condition, the construct comprising expression modules that form a regulatory circuit, the construct including: a first repressor protein expression module, wherein the first repressor protein binds a first repressor protein nucleic acid binding element and represses transcription from a coding sequence comprising the first repressor protein binding element, and wherein repression activity of the first repressor protein is sensitive to inhibition by a first exogenous agent, the presence or absence of the first exogenous agent establishing a predetermined condition;
ii) a second repressor protein expression module, wherein the second repressor protein binds a second repressor protein nucleic acid binding element and represses transcription from a coding sequence comprising the second repressor protein binding element, wherein the second repressor protein is different from the first repressor protein; and
iii) an effector expression module, comprising a nucleic acid sequence encoding an effector protein, operably linked to a genetic element comprising a binding element for the second repressor protein, such that expression of the second repressor protein causes repression of effector expression from the effector expression module, wherein the second expression module comprises a first repressor protein nucleic acid binding element that permits repression of transcription of the second repressor protein when the element is bound by the first repressor protein, the respective modules forming a regulatory circuit such that in the absence of the first exogenous agent, the first repressor protein is produced from the first repressor protein expression module and represses transcription from the second repressor protein expression module, such that repression of effector expression by the second repressor protein is relieved, resulting in expression of the effector protein, but in the presence of the first exogenous agent, the activity of the first repressor protein is inhibited, permitting expression of the second repressor protein, which maintains expression of effector protein expression in the “off” state, such that the first exogenous agent is required by the circuit to maintain effector protein expression in the “off” state, and removal or absence of the first exogenous agent defaults to expression of the effector protein.

In some embodiments, the effector is a toxin or a protein that induces a cell death program. Any protein that is toxic to the host cell can be used. In some embodiments the toxin only kills those cells in which it is expressed. In other embodiments, the toxin kills other cells of the same host organism. Any of a large number of products that will lead to cell death can be employed in a deadman kill switch. Agents that inhibit DNA replication, protein translation or other processes or, e.g., that degrade the host cell's nucleic acid, are of particular usefulness. To identify an efficient mechanism to kill the host cells upon circuit activation, several toxin genes were tested that directly damage the host cell's DNA or RNA. The endonuclease ecoRI, the DNA gyrase inhibitor ccdB and the ribonuclease-type toxin mazF were tested because they are well-characterized, are native to E. coli, and provide a range of killing mechanisms. To increase the robustness of the circuit and provide an independent method of circuit-dependent cell death, the system can be further adapted to express, e.g., a targeted protease or nuclease that further interferes with the repressor that maintains the death gene in the “off” state. Upon loss or withdrawal of the survival signal, death gene repression is even more efficiently removed by, e.g., active degradation of the repressor protein or its message. As non-limiting examples, mf-Lon protease was used to not only degrade LacI but also target essential proteins for degradation. The mf-Lon degradation tag pdt #1 can be attached to the 3′ end of five essential genes whose protein products are particularly sensitive to mf-Lon degradation, and cell viability was measured following removal of aTc. Among the tested essential gene targets, the peptidoglycan biosynthesis gene murC provided the strongest and fastest cell death phenotype (survival ratio <1×10⁻⁴ within 6 hours).

As used herein, the term “predetermined input” refers to an agent or condition that influences the activity of a transcription factor polypeptide in a known manner. Generally, such agents can bind to and/or change the conformation of the transcription factor polypeptide to thereby modify the activity of the transcription factor polypeptide. Examples of predetermined inputs include, but are not limited to, environmental input agents that are not required for the survival of a given host organism (i.e., in the absence of a synthetic biological circuit as described herein). Conditions that can provide a predetermined input include, for example temperature, e.g., where the activity of one or more factors is temperature-sensitive, the presence or absence of light, including light of a given spectrum of wavelengths, and the concentration of a gas, salt, metal or mineral. Environmental input agents include, for example, a small molecule, biological agents such as pheromones, hormones, growth factors, metabolites, nutrients, and the like and analogs thereof; concentrations of chemicals, environmental byproducts, metal ions, and other such molecules or agents; light levels; temperature; mechanical stress or pressure; or electrical signals, such as currents and voltages.

In some embodiments, reporters are used to quantify the strength or activity of the signal received by the modules or programmable synthetic biological circuits of the disclosure. In some embodiments, reporters can be fused in-frame to other protein coding sequences to identify where a protein is located in a cell or organism. Luciferases can be used as effector proteins for various embodiments described herein, for example, measuring low levels of gene expression, because cells tend to have little to no background luminescence in the absence of a luciferase. In other embodiments, enzymes that produce colored substrates can be quantified using spectrophotometers or other instruments that can take absorbance measurements including plate readers. Like luciferases, enzymes like β-galactosidase can be used for measuring low levels of gene expression because they tend to amplify low signals. In some embodiments, an effector protein can be an enzyme that can degrade or otherwise destroy a given toxin. In some embodiments, an effector protein can be an odorant enzyme that converts a substrate to an odorant product. In some embodiments, an effector protein can be an enzyme that phosphorylates or dephosphorylates either small molecules or other proteins, or an enzyme that methylates or demethylates other proteins or DNA.

In some embodiments, an effector protein can be a receptor, ligand, or lytic protein. Receptors tend to have three domains: an extracellular domain for binding ligands such as proteins, peptides or small molecules, a transmembrane domain, and an intracellular or cytoplasmic domain which frequently can participate in some sort of signal transduction event such as phosphorylation. In some embodiments, transporter, channel, or pump gene sequences are used as effector proteins. Non-limiting examples and sequences of effector proteins for use with the kill switches as described herein can be found at the Registry of Standard Biological Parts on the world wide web at parts.igem.org.

As used herein, a “modulator protein” is a protein that modulates the expression from a target nucleic acid sequence. Modulator proteins include, for example, transcription factors, including transcriptional activators and repressors, among others, and proteins that bind to or modify a transcription factor and influence its activity. In some embodiments, a modulator protein includes, for example, a protease that degrades a protein factor involved in the regulation of expression from a target nucleic acid sequence. Preferred modulator proteins include modular proteins in which, for example, DNA-binding and input agent-binding or responsive elements or domains are separable and transferrable, such that, for example, the fusion of the DNA binding domain of a first modulator protein to the input agent-responsive domain of a second results in a new protein that binds the DNA sequence recognized by the first protein, yet is sensitive to the input agent to which the second protein normally responds. Accordingly, as used herein, the term “modulator polypeptide,” and the more specific “repressor polypeptide” include, in addition to the specified polypeptides, e.g., “a LacI (repressor) polypeptide,” variants, or derivatives of such polypeptides that responds to a different or variant input agent. Thus, for a LacI polypeptide, included are LacI mutants or variants that bind to agents other than lactose or IPTG. A wide range of such agents are known in the art.

Table 4: Exemplary regulatory switches ^{b}ON switchability by an effector; other than removing the effector which confers the OFF state. ^{c}OFF switchability by an effector; other than removing the effector which confers the ON state. ^{d}A ligand or other physical stimuli (e.g., temperature, electromagnetic radiation, electricity) which stabilizes the switch either in its ON or OFF state. “refers to the reference number cited in Kis et al., J R Soc Interface. 12:20141000 (2015), where 10 both the article and the references cited therein are hereby incorporated by reference in their entireties.

TABLE 4

| | ON | OFF | | |
|---|---|---|---|---|
| name | switch^{b} | switch^{c} | origin | effector^{d} |
| ABA | yes | no | Arabidopsis thaliana, yeast | abscisic acid |
| AIR | yes | no | Aspergillus nidulans | acetaldehyde |
| ART | yes | no | Chlamydia pneumoniae | l-arginine |
| BEARON, | yes | yes | Campylobacter jejuni | bile acid |
| BEAROFF | | | | |
| BirA-tTA | no | yes | Escherichia coli | biotin (vitamin H) |
| BIT | yes | no | Escherichia coli | biotin (vitamin H) |
| Cry2-CIB1 | yes | no | Arabidopsis thaliana, yeast | blue light |
| CTA, CTS | yes | yes | Comamonasestosteronei, | food additives (benzoate, |
| | | | Homo sapiens | vanillate) |
| cTA, rcTA | yes | yes | Pseudomonas putida | cumate |
| Ecdysone | yes | no | Homo sapiens, Drosophila | Ecdysone |
| | | | melanogaster | |
| EcR:RXR | yes | no | Homo sapiens, Locusta | ecdysone |
| | | | migratoria | |
| electro-genetic | yes | no | Aspergillus nidulans | electricity, acetaldehyde |
| ER-p65-ZF | yes | no | Homo sapiens, yeast | 4,4′-dyhydroxybenzil |
| E.REX | yes | yes | Escherichia coli | erythromycin |
| EthR | no | yes | Mycobacterium tuberculosis | 2-phenylethyl-butyrate |
| GAL4-ER | yes | yes | yeast, Homo sapiens | oestrogen, 4- |
| | | | | hydroxytamoxifen |
| GAL4-hPR | yes | yes | yeast, Homo sapiens | mifepristone |
| GAL4-Raps | yes | yes | yeast, Homo sapiens | rapamycin and rapamycin |
| | | | | derivatives |
| GAL4-TR | yes | no | yeast, Homo sapiens | thyroid hormone |
| GyrB | yes | yes | Escherichia coli | coumermycin, |
| | | | | novobiocin |
| HEA-3 | yes | no | Homo sapiens | 4-hydroxytamoxifen |
| Intramer | no | yes | synthetic SELEX-derived | theophylline |
| | | | aptamers | |
| LacI | yes | no | Escherichia coli | IPTG |
| LAD | yes | no | Arabidopsis thaliana, yeast | blue light |
| LightOn | yes | no | Neurospora crassa, yeast | blue light |
| NICE | yes | yes | Arthrobacter nicotinovorans | 6-hydroxynicotine |
| PPAR | yes | no | Homo sapiens | rosiglitazone |
| PEACE | no | yes | Pseudomonas putida | flavonoids (e.g., |
| | | | | phloretin) |
| PIT | yes | yes | Streptomyces coelicolor | pristinamycin I, |
| | | | | virginiamycin |
| REDOX | no | yes | Streptomyces coelicolor | NADH |
| QuoRex | yes | yes | Streptomyces coelicolor, | butyrolactones (e.g., |
| | | | Streptomyces | SCB1) |
| | | | pristinaespiralis | |
| ST-TA | yes | yes | Streptomyces coelicolor, | γ-butyrolactone, |
| | | | Escherichia coli, Herpes | tetracycline |
| | | | simplex | |
| TIGR | no | yes | Streptomyces albus | temperature |
| TraR | yes | no | Agrobacterium tumefaciens | N-(3-oxo- |
| | | | | octanoyl)homoserine |
| | | | | lactone |
| TET-OFF, | yes | yes | Escherichia coli, Herpes | tetracycline, doxycycline |
| TET-ON | | | simplex | |
| TRT | yes | no | Chlamydia trachomatis | l-tryptophan |
| UREX | yes | no | Deinococcus radiodurans | uric acid |
| VAC | yes | yes | Caulobacter crescentus | vanillic acid |
| ZF-ER, | yes | yes | Mus musculus, Homo | 4-hydroxytamoxifen, |
| ZF-RXR/EcR | | | sapiens, Drosophila | ponasterone-A |
| | | | melanogaster | |
| ZF-Raps | yes | no | Homo sapiens | rapamycin |
| ZF switches | yes | no | Mus musculus, Homo | 4-hydroxytamoxifen, |
| | | | sapiens, Drosophila | mifepristone |
| | | | melanogaster | |
| ZF(TF)s | yes | no | Xenopus laevis, Homo | ethyl-4-hydroxybenzoate, |
| | | | sapiens | propyl-4- |
| | | | | hydroxybenzoate |
| aptamer RNAi | yes | no | synthetic SELEX-derived | theophylline |
| | | | aptamer | |
| aptamer RNAi | no | yes | synthetic SELEX-derived | theophylline |
| | | | aptamer | |
| aptamer RNAi | yes | no | synthetic SELEX-derived | theophylline, tetracycline, |
| miRNA | | | aptamer | hypoxanthine |
| aptamer Splicing | yes | yes | Homo sapiens, MS2 | MS2, p65, p50, b-catenin |
| | | | bacteriophage | |
| aptazyme | no | yes | synthetic SELEX-derived | theophylline |
| | | | aptamer, Schistosoma | |
| | | | mansoni | |
| replicon CytTS | yes | no | Sindbis virus | temperature |
| TET-OFF- | yes | yes | Escherichia coli, Herpes | doxycycline |
| shRNA, TET- | | | simplex, Homo sapiens | |
| ON-shRNA | | | | |
| theo aptamer | no | yes | synthetic SELEX-derived | theophylline |
| | | | aptamer | |
| 3′ UTR aptazyme | yes | no | synthetic SELEX-derived | theophylline, tetracycline |
| | | | aptamers, tobacco ringspot | |
| | | | virus | |
| 5′ UTR aptazyme | no | yes | synthetic SELEX-derived | theophylline |
| | | | aptamer, Schistosoma | |
| | | | mansoni | |
| Hoechst aptamer | no | yes | synthetic RNA sequence | Hoechst dyes |
| H23 aptamer | no | yes | Archaeoglobus fulgidus | L7Ae, L7KK |
| L7Ae aptamer | yes | yes | Archaeoglobus fulgidus | L7Ae |
| MS2 aptamer | no | yes | MS2 bacteriophage | MS2 |
| AID | no | yes | Arabidopsis thaliana, Oryza | auxins (e.g., IAA) |
| | | | sativa, Gossypium hirsutum | |
| ER DD | no | yes | Homo sapiens | CMP8, 4- |
| | | | | hydroxytamoxifen |
| FM | yes | no | Homo sapiens | AP21998 |
| HaloTag | no | yes | Rhodococcus sp. RHA1 | HyT13 |
| HDV-aptazyme | no | yes | hepatitis delta virus | theophylline, guanine |
| PROTAC | no | yes | Homo sapiens | proteolysis targeting |
| | | | | chimeric molecules |
| | | | | (PROTACS) |
| shield DD | yes | no | Homo sapiens | shields (e.g., Shld1) |
| shield LID | no | yes | Homo sapiens | shields (e.g., Shld1) |
| TMP DD | yes | no | Escherichia coli | trimethoprim (TMP) |

### IV. Cell-Free Methods of Making Single-Stranded DNA Molecules

Conventional methods for production of viral and virally-derived DNA typically use eukaryotic cells, e.g., mammalian or insect cells. One commonly used insect cell line is Sf9. However, not only do these cells both contain enzymes and other proteins which may have a deleterious effect on the DNA to be replicated, but the process of purifying the desired DNA from cell lysates introduces cellular nucleic acids whose presence can make purification of the desired DNA product more difficult. Furthermore, such impurities or contaminants can have a range of deleterious and/or unwanted effects in the subject to which the desired DNA is administered. Additionally, such traditional cell-based production methods can have issues with respect to the quantity of DNA vector product produced, and it is not uncommon for significant engineering of the cell line itself or the production technology to be required to produce desirable yields.

The present disclosure relates to cell-free methods of making single-stranded DNA molecules (“ssDNA”, “SSD” all of which are used interchangeably herein). The inventors of the present disclosure surprisingly found that a cell-free method as disclosed herein can be applied to produce a ssDNA molecule to a desired yield and of a desired quality. This in particular refers to a situation, where the cell-free methods of the present invention are compared to methods that rely on the use of cells to produce closed-ended DNA molecules, and to methods that produce a ssDNA molecule without the steps of a double-stranded ceDNA intermediate. Certain methods for the production of a double-stranded ceDNA vector comprising various ITR configurations using cell-based methods are described in Example 1 of International Patent Application Publication Nos. WO2019/051255 and WO2019/113310, the contents of which are incorporated by reference in their entireties herewith. Another significant advantage offered by the cell-free synthetic methods provided herein over cell-based production methods is that, in addition to the higher yield, the methods described herein are readily scalable small reactions (~1 mL) and up to at least medium (>40 mL) and further without compromising the purity.

In some aspects, the disclosure provides a method for producing a linear, single-stranded DNA (ssDNA) molecule, the method comprising contacting a double-stranded, closed-ended DNA (ceDNA) molecule with an endonuclease followed by an exonuclease, thereby producing the linear, ssDNA molecule (described in Section II herein). According to some embodiments, the method further comprises the following steps prior to the contacting step with an endonuclease: a) performing rolling circle amplification (RCA) using a double-stranded DNA (dsDNA) molecule, thereby producing an intermediate dsDNA product (described in Section III herein); and b) performing cell-free, enzymatic synthesis using the intermediate dsDNA product, thereby producing the ceDNA molecule. According to some embodiments, a further step of purifying the ceDNA molecule prior to the contacting step with an endonuclease is carried out. In some embodiments, a further step of purifying the ssDNA molecule after it is produced from the ceDNA is carried out (described herein). Each of the above steps are described in more detail in the subsections, below.

According to some embodiments, the methods and/or production steps of the present disclosure are carried out entirely in a cell-free environment. According to some embodiments, the methods and/or production steps of the present disclosure are carried out partially in a cell-free environment. According to some embodiments, the ssDNA molecule is synthetically produced in vitro. According to some embodiments, the ssDNA molecule is synthetically produced in vitro in a cell free environment.

A. Production of Single-Stranded DNA from Closed-Ended DNA (ceDNA)

In some aspects, the disclosure provides a method for producing a linear, single-stranded DNA (ssDNA) molecule, the method comprising contacting a double-stranded, closed-ended DNA (ceDNA) molecule with an endonuclease followed by an exonuclease, thereby producing the linear, ssDNA molecule.

### (i) Endonuclease Step

In some embodiments, the ceDNA molecule is contacted with an endonuclease.

According to some embodiments, the endonuclease is Endonuclease V. Endonuclease V, often called deoxyinosine 3′ endonuclease, recognizes DNA containing deoxyinosines (paired or not) on double-stranded DNA, single-stranded DNA with deoxyinosines and to a lesser degree, DNA containing abasic sites (ap) or urea, base mismatches, insertion/deletion mismatches, hairpin or unpaired loops, flaps and pseudo-Y structures. Endonuclease V cleaves the second phosphodiester bonds 3′ to the mismatch of deoxyinosine (Yao, M. and Kow, Y. W. (1995). J. Biol. Chem. 270, 28609-28616), leaving a nick with 3′-hydroxyl and 5′-phosphate (He, B., Qing, H. and Kow, Y. W. (2000). Mutat. Res. 459, 109-114).

In other embodiments, the endonuclease is Nb.BbvCI. In one embodiment, the endonuclease is Nb.BsmI. In one embodiment, the endonuclease is Nb.BsrDI. In one embodiment, the endonuclease is Nb.BssSI. In one embodiment, the endonuclease is Nb.BtsI. In one embodiment, the endonuclease is Nt.AlwI. In one embodiment, the endonuclease is Nt.BbvCI. In one embodiment, the endonuclease is Nt.BsmI. In one embodiment, the endonuclease is Nt.BspQI. In one embodiment, the endonuclease is Nt.BstNBI. In one embodiment, the endonuclease is Nt.CviPII. In one embodiment, the endonuclease is Endonuclease V (Endo V).

According to further embodiments, the endonuclease has enzymatic activity on a uridine-, inosine-containing residue. In one embodiment, the endonuclease has enzymatic activity on a xanthosine-containing residue. In one embodiment, the endonuclease has enzymatic activity on an oxanosine-containing residue. According to some embodiments, the endonuclease having enzymatic activity on uridine-, inosine-, xanthosine-, and/or oxanosine-containing residue can nick the modified DNA at the second phosphodiester bond 3′ to a lesion.

According to some embodiments, the ceDNA comprises a nickase recognition sequence (“nick site”) for an endonuclease. In one embodiment, the ceDNA comprise a terminal resolution site (trs) sequence of an AAV ITR that contains a nick site for an endonuclease. According to some embodiments, the ceDNA comprises one or more recognition nucleotide sequences of one or more nicking endonucleases that are each independently selected from Nb.BbvCI, Nb.BsmI, Nb.BsrDI, Nb.BssSI, Nb.BtsI, Nt.AlwI, Nt.BbvCI, Nt.BsmI, Nt.BspQI, Nt.BstNBI, Nt.CviPII, and an isoschizomer of any of the foregoing. According to a further embodiment, the one or more recognition nucleotide sequences comprise any one or more of the following sequences shown in Table 5 below:

TABLE 5

| Recognition | |
|---|---|
| Nucleotide | |
| Sequence | Nicking Endonuclease |
| 5′-GCTGAGG-3′ | (Nb.BbvCI) |
| 5′ NGCATTC-3′ | (Nb.BsmI) N can |
| | be G, C, A, or T |
| 5′-NNCATTGC-3′ | (Nb.BsrDI) |
| 5′-CTCGTG-3′ | (Nb.BssSI) |
| 5′-NNCACTGC-3′ | (Nb.BtsI) |
| 5′-GGATCNNNNN-3′ | (Nt.AlwI) |
| 5′-CCTCAGC-3′ | (Nt.BbvCI) |
| 5′-GTCTCNN-3′ | (Nt.BsmI) |
| 5′-GTCTCNN-3′ | (Nt.BsmI) |
| 5′-GCTCTTCN-3′ | (Nt.BspQI) |
| 5′-GAGTCNNNNN-3′ | (Nt.BstNBI) |
| 5′-CCD-3′ | (Nt.CviPII) D |
| | can be A or G or T |

According to some embodiments, the one or more recognition nucleotide sequences are each an engineered sequence. According to further embodiments, the one or more recognition nucleotide sequences each comprise one or more nick sites of the one or more nicking endonucleases. According to some embodiments, the 3′ terminal portion of the double-stranded ceDNA molecule comprises a nickase recognition sequence. In one embodiment, the 3′ terminal portion of the ceDNA molecule comprises the sequence 5′-CCAA-3′. In some embodiments the 3′ terminal portion of the ceDNA molecule comprises any one or more of the sequences shown in Table 6 below. Further, since these are unique sequences after a double-stranded ceDNA with special engineered nick sites has been nicked by a nicking endonuclease as shown in Table 6, resultant ssDNA molecules also comprise any one or more of the sequences shown in Table 6 below in its 3′ terminal fragment.

TABLE 6

| Sequence | Nicking Endonuclease |
|---|---|
| 5′-CCAA-3′ | (Nb.BtsI) (Nb.BsrDI) |
| | (Nt.CviPII) |
| 5′-CCAAGC-3′ | (Nb.BbvCI) |
| 5′-CCAACC-3′ | (Nt.BbvCI) |
| 5′-CCAAGAGT | (Nt.BstNBI)- |
| CNNNN-3′ | N can be A, G, C, or T |
| 5′-CCAAG-3′ | (Nb.BsmI) |
| 5′-CCAAC-3′ | (Nb.BssSI) |
| 5′-CCAAGGAT | (Nt.AlwI) |
| CNNNN-3′ | |
| 5′-CCAAGTCTCN-3′ | (Nt.BsmAI) |
| 5′-CCAAGCTCTTCN-3′ | (Nt.BspQI) |

According to some embodiments, the one or more nick sites are about 0 to about 20 nucleotides downstream of a terminal resolution site (trs), for example, about 0, 1, 2, 3, 4, 5, 6, 7, 8, 9, 10, 11, 12, 13, 14, 15, 16, 17, 18 19, or 20 nucleotides downstream of the terminal resolution site (trs), or for example about 0 to about 15, about 0 to 10, about 0 to 5, about 5 to 15, about 10 to 20, about 15 to 20, about 10 to 20, about 5 to 20 nucleotides downstream of the terminal resolution site (trs). According to some embodiments, there is just one nick site that serves as the exonuclease entry site.

In some embodiments, a double-stranded ceDNA molecule may comprise more than one nick site. For example, nick sites may be located in a sense strand 5′ of the nucleic acid sequence of interest. In another embodiment, nick sites may be located within the nucleic acid sequence of interest. In other embodiments, a double-stranded ceDNA molecule may comprise multiple nick sites 3′ and/or 5′ of the nucleic acid sequence of interest, and/or within the nucleic sequence of interest. In some embodiments, a nick site is located adjacent to and/or upstream of a promoter and/or TSS.

According to some embodiments, the ceDNA construct comprises one or more recognition nucleotide sequences of Nb.BbvCI or an isoschizomer thereof. According to some embodiments, the ceDNA construct comprises a single recognition nucleotide sequence of Nb.BbvCI or an isoschizomer thereof. According to some embodiments, the ceDNA construct comprises one or more recognition nucleotide sequences of Nb.BtsI or an isoschizomer thereof. According to some embodiments, the ceDNA construct comprises a single recognition nucleotide sequence of Nb.BtsI or an isoschizomer thereof. According to some embodiments, the ceDNA construct comprises one or more recognition nucleotide sequences of Endonuclease V or an isoschizomer thereof.

In some embodiments, a further step of purifying the ceDNA molecule prior to the contacting step with an endonuclease is carried out. For example, the ceDNA may be purified prior to the contacting step with an endonuclease if the ceDNA was produced using rolling circle amplification (as described herein in Section IV (B)) and enzymatic synthesis (as described herein in Section IV (C)).

### (ii) Exonuclease Step

In some embodiments, the ceDNA molecule is contacted with an exonuclease after it was contacted with the endonuclease. The exonuclease is capable of removing the nicked strand of the ceDNA construct, beginning at the one or more nick sites and ending at the one or more phosphorothioate-modified nucleotides or another one or more nick sites. The exonuclease can be selected from, but is not limited to T7 exonuclease, Lambda exonuclease, T5 exonuclease, Exonuclease V, and Exonuclease III.

In one embodiment, the exonuclease is T7 exonuclease. In one embodiment, the exonuclease is Lambda exonuclease. In one embodiment, the exonuclease is T5 exonuclease. In one embodiment, the exonuclease is Exonuclease V. In one embodiment, the exonuclease is Exonuclease III.

As discussed more extensively in Section III (B) herein, the double-stranded closed-ended DNA may comprise phosphorothioate (PS) bonds. The PS bond substitutes a sulfur atom for a non-bridging oxygen in the phosphate backbone of an oligonucleotide. Advantageously, this modification renders the internucleotide linkage resistant to nuclease degradation, and provides accuracy for targeting of the exonuclease. More specifically, this modification is advantageously located in the ITR region in a space where the exonuclease is active, and functions as a lock on the 5′ and/or 3′ ends, rendering the internucleotide linkage resistant to nuclease degradation, and ensuring the accuracy of exonuclease activity.

According to some embodiments, in the methods of production of the single-stranded DNA (ss DNA) molecule, the PS bond substitutes a sulfur atom for a non-bridging oxygen in the phosphate backbone of an oligonucleotide. Advantageously, this modification stabilizes the nucleic acids and renders the internucleotide linkage resistant to nuclease degradation.

According to some embodiments, exonuclease procession can be terminated through the inclusion of a structured region located in at least one strand of double-stranded ceDNA molecule. In some embodiments, the structured region is a stem-loop structure. In some embodiments, the structured region is a bubble. In some embodiments, the structured region is a loop.

In some embodiments, a structured region is located near or adjacent to a stem-loop structure that will become the 5′ stem-loop structure in an ssDNA molecule produced by the methods disclosed herein.

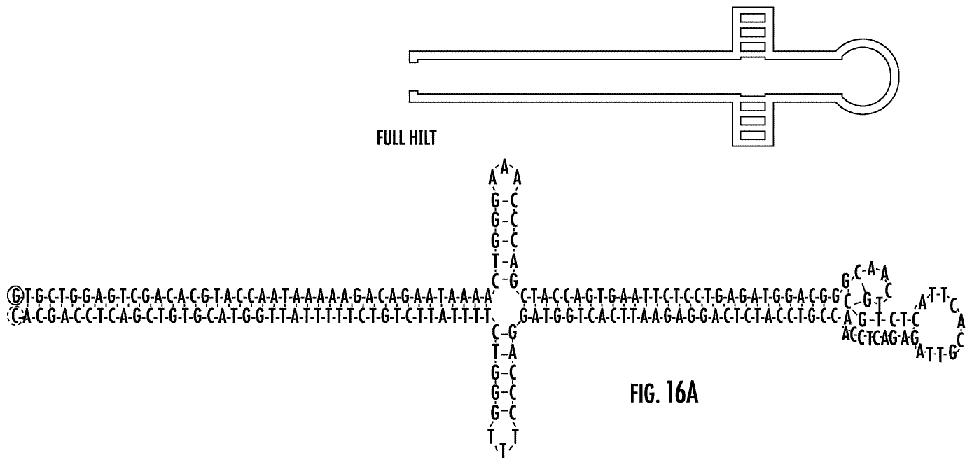

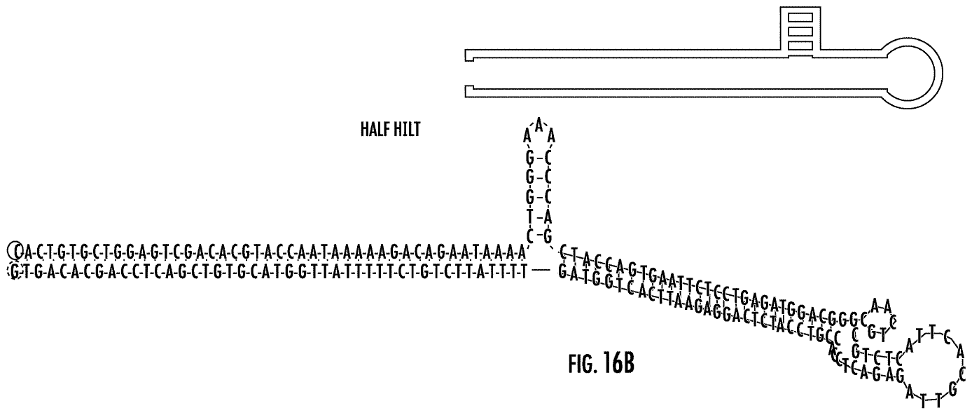

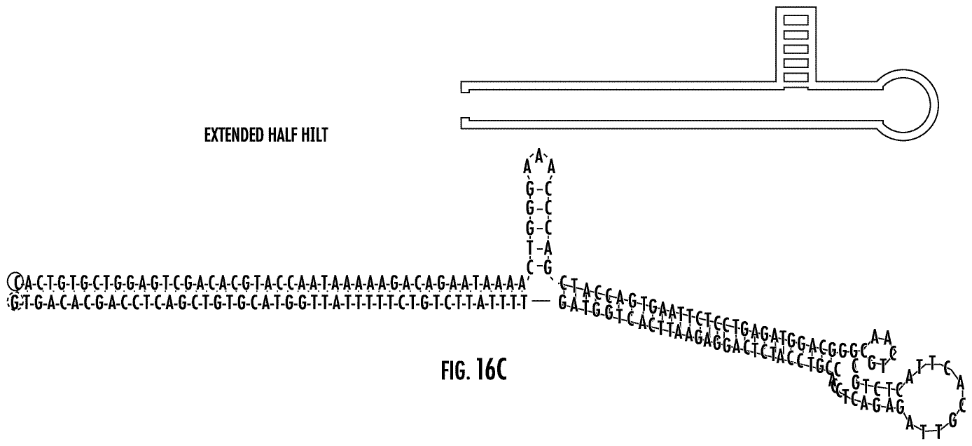

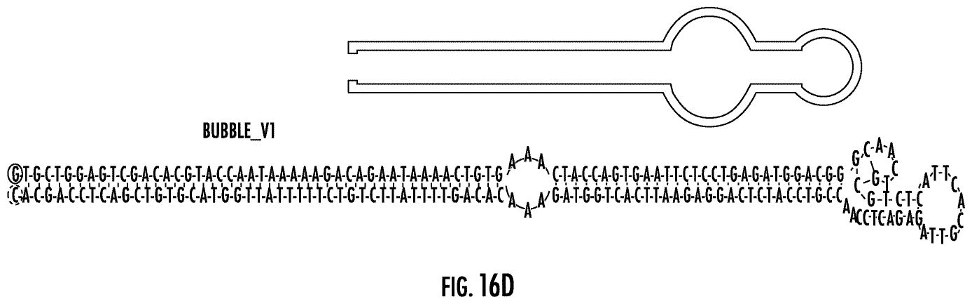

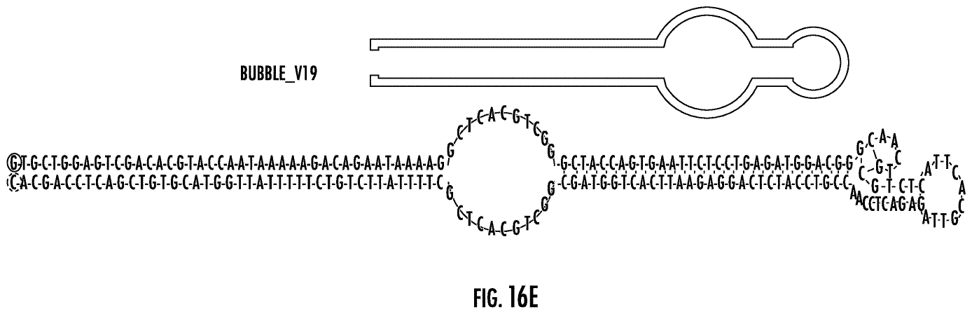

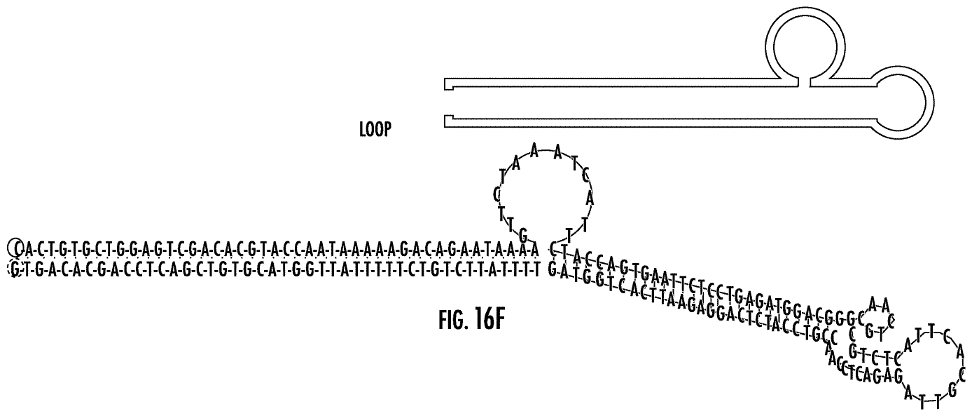

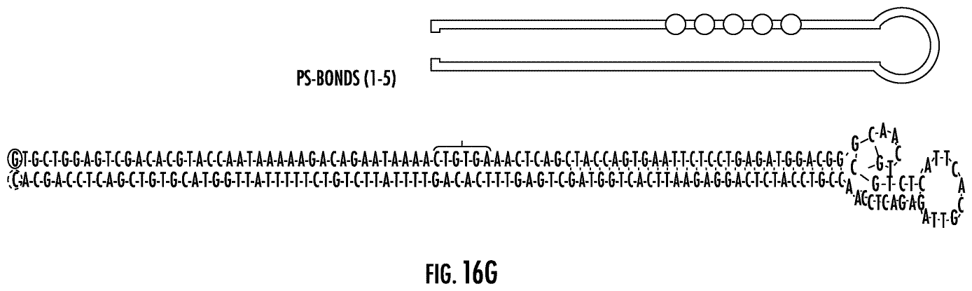

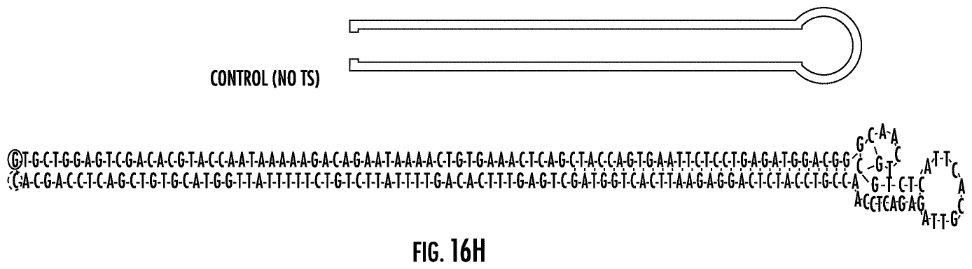

In some embodiments, a structured region is a “full hilt” structure, which comprises two stem-loop structures on opposite strands of a double-stranded ceDNA molecule (see, for example, FIG. 16A). In some embodiments, the length of each stem in a full hilt structure is at least 3, 4, 5, 6, 7, 8, 9, 10, 15, 20, 25, 30, 35, 40, 45, 50 or more base pairs. In some embodiments, the length of each loop in a full-hilt structure is at least 3, 4, 5, 6, 7, 8, 9, 10, 15, 20, 25, 30, 35, 40, 45, 50 or more unpaired nucleotides.

In some embodiments, a structured region is a “half hilt” structure, which comprises one stem-loop structure on one strand of a double-stranded ceDNA molecule (see, for example, FIG. 16B). In some embodiments, the length of each stem in a half hilt structure is at least 3, 4, 5, 6, 7, 8, 9, 10, 15, 20, 25, 30, 35, 40, 45, 50 or more base pairs. In some embodiments, the length of each loop in a full-hilt structure is at least 3, 4, 5, 6, 7, 8, 9, 10, 15, 20, 25, 30, 35, 40, 45, 50 or more unpaired nucleotides. In some embodiments wherein the stem is at least 8 base pairs in length, a half hilt structure may also be referred to herein as an “extended half hilt” (see, for example, FIG. 16C).

In some embodiments, a structured region may be referred to as a “bubble” structure, which comprises two unpaired regions on opposite strands of a double-stranded ceDNA molecule, which are flanked on both sides by double-stranded DNA (see, for example, FIGS. 16D and 16E). In some embodiments, the length of unpaired nucleotides in a bubble structure is at least 4, 5, 6, 7, 8, 9, 10, 15, 20, 25, 30, 35, 40, 45, 50 or more base pairs.

In some embodiments, a structured region may be referred to as a “loop” structure, which comprises a single unpaired region that loops out of one stranded of a double-stranded ceDNA molecule, which is flanked on both sides by double-stranded DNA (see, for example, FIG. 16F). In some embodiments, the length of unpaired nucleotides in a loop structure is at least 4, 5, 6, 7, 8, 9, 10, 15, 20, 25, 30, 35, 40, 45, 50 or more base pairs.

According to some embodiments, the structured region used to terminate the exonuclease procession remains in the ssDNA molecule after completion of the exonuclease reaction. In some embodiments, the structured region may be removed by other enzymatic means.

B. Production of a dsDNA Intermediate Using Rolling Circle Amplification (RCA)

According to some embodiments, the method described in Section IV (A) further comprises the following steps prior to the contacting step with an endonuclease: a) performing rolling circle amplification (RCA) using a double-stranded DNA (dsDNA) molecule, e.g., a plasmid, thereby producing a first intermediate molecule, e.g., an intermediate dsDNA molecule, e.g., a dsDNA molecule which is not a closed-ended DNA molecule; followed by b) performing cell-free, enzymatic synthesis using the first intermediate dsDNA molecule, thereby producing a second intermediate molecule, e.g., an intermediate ceDNA molecule.

In one embodiment, the first dsDNA intermediate is generated using rolling circle amplification (RCA) of a template, e.g., a plasmid template, to produce a first intermediate, e.g., dsDNA intermediate. In one embodiment, the dsDNA intermediate is not a closed-ended DNA. According to some embodiments, the RCA step comprises contacting the dsDNA molecule with a primer and a DNA polymerase.

The term “plasmid DNA” refers to a circular nucleic acid molecule, preferably to an artificial nucleic acid molecule. Such plasmid DNA constructs may be storage vectors, expression vectors, cloning vectors, transfer vectors, etc. Preferably, a plasmid DNA within the meaning of the present invention comprises in addition to the elements described herein, optionally a selection marker, such as an antibiotic resistance factor, and a sequence suitable for multiplication of the vector, such as an origin of replication. Typical plasmid backbones are, e.g., pUC19 and pBR322.

RCA uses circular DNA (e.g., a plasmid) as template and random hexamer primers that anneal to the circular template DNA at multiple sites. No sequence-specific primers are thus required. This reaction requires the two components: (a) a free 3′ end, and (b) a rolling circle polymerase. Usually, Phi29 DNA polymerase is used to extend each of the primers. The reaction is performed at 30° C., and thus without the need for thermocycling (i.e., the use of different temperatures for different steps). When the DNA polymerase reaches a downstream-extended primer, strand displacement synthesis occurs and the displaced strand is rendered single-stranded and available to be primed by more hexameric primers. This process continues and results in exponential, isothermal amplification.

A number of references disclose primer, primer design, and amplification techniques, including U.S. Pat. Nos. 5,871,921, 5,648,245, 5,866,377 and 5,854,033, all hereby incorporated by reference. RCA is described in, e.g., Dean et al. (Genome Res. 2001 June; 11 (6): 1095-9) and Kumar and Chernaya (Biotechniques. 2009 July; 47 (1): 637-9), the contents of which are incorporated by reference in their entireties herein.

C. Production of a Closed-Ended DNA from a Double-Stranded DNA Intermediate

As described herein, the first intermediate dsDNA molecule produced using, e.g., rolling circle amplification (described in Section IV (B)) is subjected to a further step of cell-free, enzymatic synthesis to produce a second intermediate, e.g., a double-stranded closed-ended DNA (ceDNA) molecule.

A cell-free process for the production of double-stranded ceDNA is described in International Patent Application No. PCT/US2022/053868 (published as International patent publication No. WO 2023122303 A3), the contents of which are incorporated by reference in its entirety herein.

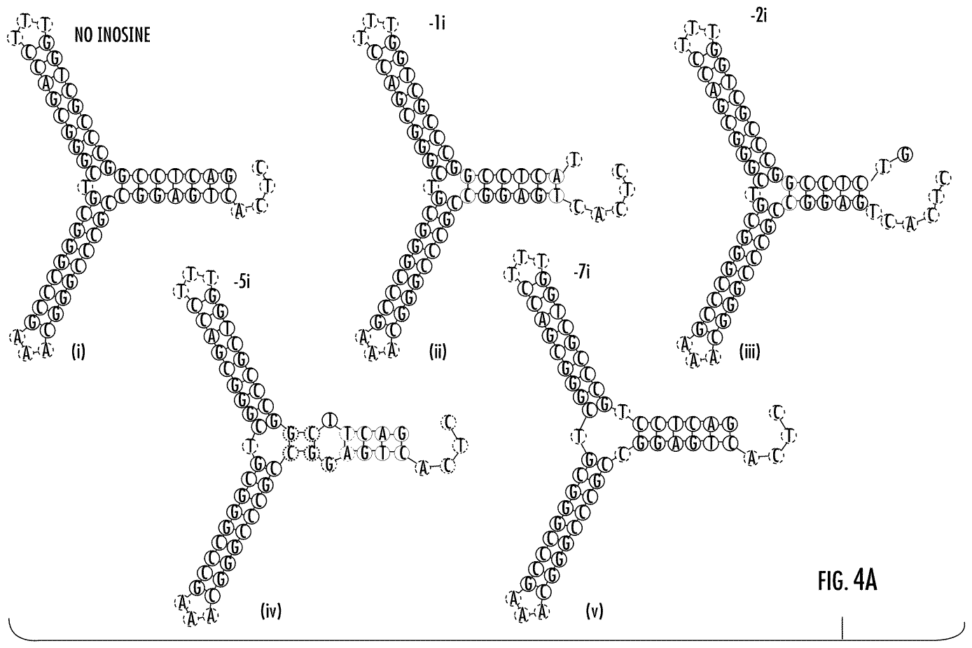

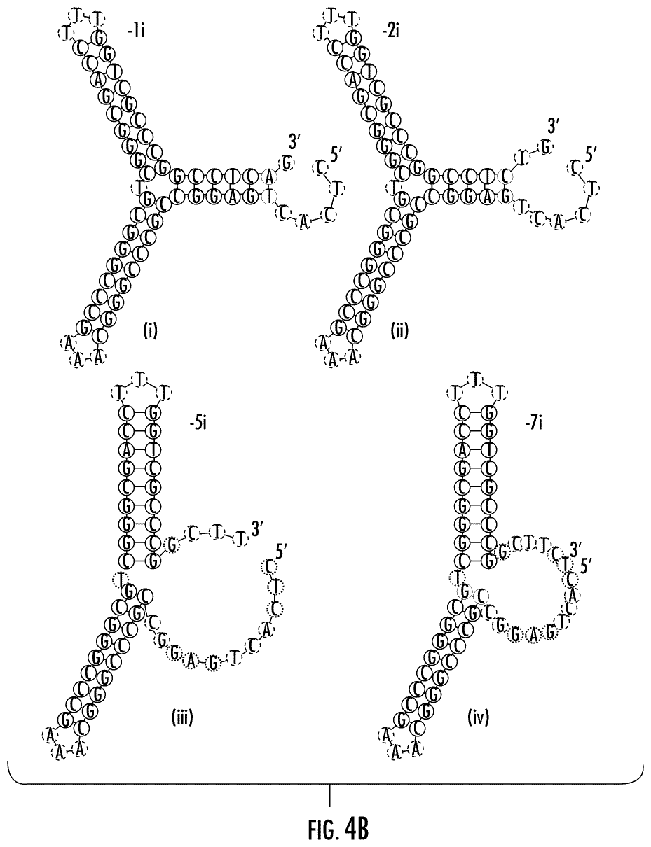

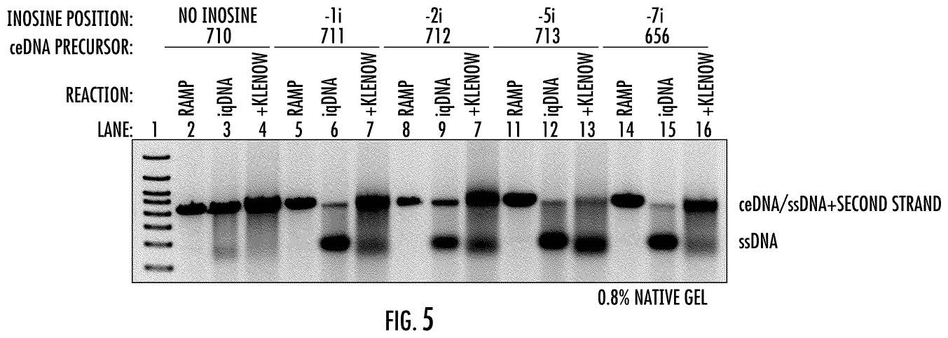

An overview of an exemplary embodiment of cell-free synthetic method of preparing a ceDNA vector is illustrated in FIG. 4 of International Patent Application No. PCT/US2022/053868 (published as International patent publication No. WO 2023122303 A3). Briefly, the transgene expression cassette (in diagonal stripes) is excised from a double-stranded DNA construct using at least one restriction endonuclease, followed up by ligation of the insert with inverted terminal repeat (ITR) oligonucleotides to form ceDNA. ITR oligonucleotides are single-stranded oligonucleotides that self-anneal to form an ITR-like three-dimensional configuration. Restriction endonucleases used in the methods described herein, such as but not necessarily limited to Type IIS restriction endonucleases, cleave the DNA at a site that is distinct and not within the recognition site. These restriction endonucleases used in the cell-free synthetic methods disclosed herein also recognize non-palindromic nucleotide sequences such that the recognition sequence (which is also the binding site) for the enzyme is only encoded on one strand (see e.g., FIG. 5 of International Patent Application No. PCT/US2022/053868, published as International patent publication No. WO 2023122303 A3). Therefore, cleavage by this class of restriction endonucleases is directional, occurring either upstream or downstream of the recognition site, but not within the recognition site itself, unlike other restriction endonucleases that are most heavily used in molecular biology such as EcoRI (see FIG. 5 of International Patent Application No. PCT/US2022/053868, published as International patent publication No. WO 2023122303 A3). The strand that encodes the recognition sequence dictates which side (i.e., downstream or upstream) of the sequence is cleaved. Taken together, the unique activity of the restriction endonucleases used in the methods described herein allows any sequence within a pre-determined distance from a specific recognition site to be cleaved by the restriction endonuclease and consequently, any overhang sequence to be generated. Digestion with the special restriction endonuclease(s) creates cohesive overhangs at both 5′ and 3′ ends of the excised insert that are compatible with the overhangs of the ITR oligonucleotide. In other words, the design of the ITR oligonucleotide and insert overhangs drives the high specificity of the ligation process such that the ITR oligonucleotide overhangs and the insert overhangs are compatible with each other. Once ligated, the desired ceDNA product is not susceptible to digestion with the restriction endonuclease because the recognition site is not re-generated. However, in situations where the excised insert and the plasmid fragments re-ligate into the original construct, the recognition sites are re-generated and therefore allow the construct to be cleaved.

In some embodiments, the intermediate dsDNA molecule produced by digestion with a restriction endonuclease may be referred to herein as a “cleaved dsDNA molecule” or a “cleaved intermediate dsDNA molecule”.

In some embodiments, a double-stranded closed-ended DNA vector is generated by excising a transgene expression cassette from a double-stranded (ds) DNA (dsDNA) construct, followed by ligation of the ends of the insert to a first oligonucleotide comprising one or more hairpin structures and a second oligonucleotide comprising one or more hairpin structures to form the ds ceDNA. In some embodiments, each of oligonucleotides independently includes 1, 2, 3, 4, or more stem-loop regions. In some embodiments, each of the oligonucleotides independently includes 2 or 3 stem-loop regions. In some embodiments, the first oligonucleotide comprising one or more hairpin structures and the second oligonucleotide comprising one or more hairpin structures are each a single-stranded oligonucleotide that self-anneals to form a three-dimensional configuration. In further embodiments, the three-dimensional configuration is a T- or Y-shaped stem-loop structure.

In another aspect, a dsDNA (e.g., ceDNA) is generated by excising a transgene expression cassette from a double-stranded DNA construct, followed by ligation of the ends of the insert to ITR oligonucleotides to form the ds ceDNA. The ligation may be effected by a ligase (e.g., T4 ligase) or an AAV Rep protein. In one embodiment, the reaction mixture is not purified prior to ligation. In such an embodiment, the excision of the transgene expression cassette (e.g., with one or more restriction endonucleases) and ligation take place simultaneously in a single reaction vessel. In an alternative embodiment, the reaction mixture is purified prior to ligation.

In one embodiment, the restriction endonuclease(s) used in the synthetic methods provided herein is a Type IIS restriction endonuclease. Non-limiting examples of Type IIS restriction endonucleases include AcuI, AlwI, Alw26I, BasI, BbsI, BbvI, BceAI, BcgI, BCiVI, BcoDI, BruAI, BmrI, BpiI, BpuEI, BsaI, BsaXI, BseGI, BseRI, BsgI, BsmAI, BsmBI, BsmFI, BsmI, BspCNI, BspMI, BspQI, BsrDI, BsrI, BtgZI, BtsCI, BtsI, MutI, CspCI, EarI, EciI, Eco31I, Esp3I, FauI, FokI, HgaI, HphI, HpyAV, LguI, MboII, MlyI, MmeI, MnlI, Mva12691, NmeAIII, PaqCI, PleI, SapI, SfaNI, and an isoschizomer of any of the foregoing. Isoschizomers are pairs of restriction endonucleases that are specific to the same recognition sequence. For example, BcoDI and BsmAI are isoschizomers of each other, both being specific to the recognition sequence of 5′-GTCTC-3′. In one embodiment, the Type IIS endonuclease(s) is selected from BbsI, BsaI, Esp3I, and SapI, and an isoschizomer thereof. In one embodiment, the Type IIS endonuclease is BbsI or an isoschizomer thereof. In one embodiment, the Type IIS endonuclease is BsaI or an isoschizomer thereof. In one embodiment, the Type IIS endonuclease is BbsI or an isoschizomer thereof. In one embodiment, the Type IIS endonuclease is Esp3I or an isoschizomer thereof. In one embodiment, the Type IIS endonuclease is SapI or an isoschizomer thereof.

### D. Isolation and Purification

The single-stranded DNA (ssDNA) molecules as described herein are advantageous over other vectors in that they can be used more safely to express a transgene in a cell, tissue or subject, as compared to DNA vectors produced in a cell culture environment (e.g., an insect cell line such as the Sf9 cell line, yeast cells, or mammalian cell lines such as HEK 293). That is, undesirable side effects can potentially be minimized by generating the linear vectors by such cell-free methods since the resulting vectors are free of bacterial or insect cell contaminants. The synthetic production methods may also result in greater purity of the desired vector. The synthetic production method may also be more efficient and/or cost effective than traditional cell-based production methods for such vectors. The vectors synthesized as described herein can express any desired transgene, for example, a transgene to treat or cure a given disease. One of ordinary skill in the art will readily recognize that any transgene used in conventional gene therapy methods with conventional recombinant vectors can be adapted for expression by e.g., single-stranded DNA (ssDNA) molecules made by the methods described herein, particularly without limitations of the size capacity of a transgene insert.

In the present disclosure, it is to be understood that the production process of the present disclosure can be potentially conducted in an entirely cell-free environment if it is desired. However, depending on a starting material, some DNA components can be derived from nucleotides fragment originally prepared in a cell (e.g., plasmid-ceDNA, AAV vectors produced from insect cells).

It will be appreciated by one of ordinary skill in the art that one or more enzymes for the synthetic production method or one or more of the oligonucleotide components can be produced from a cell and used in the methods of the disclosure in purified form. Accordingly, in some embodiments, the synthetic production method is a cell-free method, however, a restriction enzyme and/or ligase enzyme can be produced from a cell.

In one embodiment, a restriction endonuclease and/or a ligation-competent protein can be expressed or provided from an expression vector in a cell, e.g., bacterial cell. In one embodiment, a cell, such as a bacterial cell, comprising an expression vector expressing one or more of the restriction endonucleases or the ligase enzymes can be present. Therefore, while the methods disclosed herein are primarily directed to cell-free synthetic methods to generate the ssDNA molecules disclosed herein, also encompassed in some embodiments are synthetic production methods where a cell, e.g., a bacterial cell, but not an insect cell, is present and can be used to express one or more of the enzymes required in the method. In such embodiments, the cell expressing a restriction endonuclease and/or ligation-competent protein is not an insect cell. In all embodiments where a cell is present and expresses one or more restriction endonucleases or ligation-competent proteins, the cell does not replicate the single-stranded DNA (ssDNA) molecule. Stated differently, the intracellular machinery of the cell does not replicate, or is not involved in the replication of the single-stranded DNA (ssDNA) molecule.

Methods to generate and isolate a single-stranded DNA (ssDNA) molecule are described herein. For example, single-stranded DNA (ssDNA) molecules as described herein produced by the synthetic methods described herein can be harvested or collected at an appropriate time and can be optimized to achieve a high-yield production of the vectors. ssDNA molecules can be purified by any means known to those of skill in the art for purification of DNA. In one embodiment, ssDNA molecules are purified as DNA molecules. Generally, any art-known nucleic acid purification methods can be adopted, as well as commercially available DNA extraction kits.

Purification can be implemented by subjecting a reaction mixture to chromatographic separation. As one non-limiting example, the process can be performed by loading the reaction mixture on an ion exchange column (e.g., SARTOBIND Q®) which retains nucleic acids, and then eluting (e.g., with a 1.2 M NaCl solution) and performing a further chromatographic purification on a gel filtration column (e.g., 6 fast flow GE). The DNA vector is then recovered by, e.g., precipitation.

The presence of the ssDNA molecule can be readily confirmed by digesting the vector DNA with a restriction enzyme having a single recognition site on the DNA vector and analyzing both digested and undigested DNA material using gel electrophoresis to confirm the presence of characteristic bands of linear and continuous DNA as compared to linear and non-continuous single strand DNA as known in the art.

In some embodiments, the ssDNA molecule can be delivered to a target cell in vitro or in vivo by various suitable methods as discussed herein. Vectors alone can be applied or injected. Vectors can be delivered to a cell without the help of a transfection reagent or other physical means. Alternatively, vectors can be delivered using a transfection reagent or other physical means that facilitates entry of DNA into a cell, e.g., liposomes, alcohols, polylysine-rich compounds, arginine-rich compounds calcium phosphate, microvesicles, microinjection, and the like.

According to some aspects, the disclosure provides a linear, single-stranded DNA (ssDNA) molecule comprising at least one nucleic acid sequence of interest flanked by at least one stem-loop structure at the 3′ end, produced by the method described herein. According to some embodiments, the ssDNA molecule further comprises at least one stem-loop structure at the 5′ end. According to further embodiments, the stem-loop structure at the 3′ end comprises a first inverted terminal repeat (ITR), and the stem-loop structure at the 5′ end comprises a second ITR. In some embodiments, the stem-loop structure at the 3′ end comprises one or more aptamers. In some other embodiments, the stem-loop structure at the 5's end comprises one or more aptamers. In some other embodiments, the stem-loop structures at the 3′ and 5′ ends comprise one or more aptamers. In some other embodiments, the stem-loop structures at the 3′ and 5's ends are devoid of virally derived sequences. In one embodiment, the stem-loop structures at the 3′ and 5′ ends do not comprises the 20 nt long D(−) and D(+) sequences or any transcription binding site.

### V. Pharmaceutical Compositions

In another aspect, pharmaceutical compositions are provided. The pharmaceutical composition comprises a single-stranded DNA (ssDNA) molecule described herein and a pharmaceutically acceptable carrier or diluent.

A single-stranded DNA (ssDNA) molecule described herein can be incorporated into pharmaceutical compositions suitable for administration to a subject for in vivo delivery to cells, tissues, or organs of the subject. Typically, the pharmaceutical composition comprises a single-stranded DNA (ssDNA) molecule as disclosed herein and a pharmaceutically acceptable carrier. For example, a single-stranded DNA (ssDNA) molecule can be incorporated into a pharmaceutical composition suitable for a desired route of therapeutic administration (e.g., parenteral administration). Passive tissue transduction via high pressure intravenous or intra-arterial infusion, as well as intracellular injection, such as intranuclear microinjection or intracytoplasmic injection, are also contemplated. Pharmaceutical compositions for therapeutic purposes can be formulated as a solution, microemulsion, dispersion, liposomes, or other ordered structure suitable to synthetically produced single-stranded DNA (ssDNA) molecule concentration. Sterile injectable solutions can be prepared by incorporating the synthetically produced single-stranded DNA (ssDNA) molecule in the required amount in an appropriate buffer with one or a combination of ingredients enumerated above, as required, followed by filtered sterilization including a single-stranded DNA (ssDNA) molecule can be formulated to deliver a transgene in the nucleic acid to the cells of a recipient, resulting in the therapeutic expression of the transgene or donor sequence therein. The composition can also include a pharmaceutically acceptable carrier.

Pharmaceutically active compositions comprising a single-stranded DNA (ssDNA) molecule can be formulated to deliver a transgene for various purposes to the cell, e.g., cells of a subject.

Pharmaceutical compositions for therapeutic purposes typically must be sterile and stable under the conditions of manufacture and storage. The composition can be formulated as a solution, microemulsion, dispersion, liposomes, or other ordered structure suitable to high synthetically produced single-stranded DNA (ssDNA) molecule concentration. Sterile injectable solutions can be prepared by incorporating the synthetically produced single-stranded DNA (ssDNA) molecule described herein in the required amount in an appropriate buffer with one or a combination of ingredients enumerated above, as required, followed by filtered sterilization.

A single-stranded DNA (ssDNA) molecule described herein can be incorporated into a pharmaceutical composition suitable for topical, systemic, intra-amniotic, intrathecal, intracranial, intra-arterial, intravenous, intralymphatic, intraperitoneal, subcutaneous, tracheal, intra-tissue (e.g., intramuscular, intracardiac, intrahepatic, intrarenal, intracerebral), intrathecal, intravesical, conjunctival (e.g., extra-orbital, intraorbital, retroorbital, intraretinal, subretinal, choroidal, sub-choroidal, intrastromal, intracameral and intravitreal), intracochlear, and mucosal (e.g., oral, rectal, nasal) administration. Passive tissue transduction via high pressure intravenous or intraarterial infusion, as well as intracellular injection, such as intranuclear microinjection or intracytoplasmic injection, are also contemplated.

In some aspects, the methods provided herein comprise delivering one or more single-stranded DNA (ssDNA) molecules described herein to a host cell. Also provided herein are cells produced by such methods, and organisms (such as animals, plants, or fungi) comprising or produced from such cells. Methods of delivery of nucleic acids can include lipofection, nucleofection, microinjection, biolistics, liposomes, immunoliposomes, polycation or lipid: nucleic acid conjugates, naked DNA, and agent-enhanced uptake of DNA. Lipofection is described in e.g., U.S. Pat. Nos. 5,049,386, 4,946,787; and 4,897,355) and lipofection reagents are sold commercially (e.g., TRANSFECTAM™ and LIPOFECTIN™). Delivery can be to cells (e.g., in vitro or ex vivo administration) or target tissues (e.g., in vivo administration).

Various techniques and methods are known in the art for delivering nucleic acids to cells. For example, a single-stranded DNA (ssDNA) molecule described herein can be formulated into lipid nanoparticles (LNPs), lipidoids, liposomes, lipid nanoparticles, lipoplexes, or core-shell nanoparticles. Typically, LNPs are composed of nucleic acid (e.g., ssDNA molecules as described herein) molecules, one or more ionizable or cationic lipids (or salts thereof), one or more non-ionic or neutral lipids (e.g., a phospholipid), a molecule that prevents aggregation (e.g., PEG or a PEG-lipid conjugate), and optionally a sterol (e.g., cholesterol).

Another method for delivering a single-stranded DNA (ssDNA) molecule to a cell is by conjugating the nucleic acid with a ligand that is internalized by the cell. For example, the ligand can bind a receptor on the cell surface and internalized via endocytosis. The ligand can be covalently linked to a nucleotide in the nucleic acid. Exemplary conjugates for delivering nucleic acids into a cell are described, example, in WO2015/006740, WO2014/025805, WO2012/037254, WO2009/082606, WO2009/073809, WO2009/018332, WO2006/112872, WO2004/090108, WO2004/091515 and WO2017/177326.

Single-stranded DNA (ssDNA) molecules described herein can also be delivered to a cell by transfection. Useful transfection methods include, but are not limited to, lipid-mediated transfection, cationic polymer-mediated transfection, or calcium phosphate precipitation. Transfection reagents are well known in the art and include, but are not limited to, TurboFect Transfection Reagent (Thermo Fisher Scientific), Pro-Ject Reagent (Thermo Fisher Scientific), TRANSPASS™ P Protein Transfection Reagent (New England Biolabs), CHARIOT™ Protein Delivery Reagent (Active Motif), PROTEOJUICE™ Protein Transfection Reagent (EMD Millipore), 293fectin, LIPOFECTAMINE™ 2000, LIPOFECTAMINE™ 3000 (Thermo Fisher Scientific), LIPOFECTAMINE™ (Thermo Fisher Scientific), LIPOFECTIN™ (Thermo Fisher Scientific), DMRIE-C, CELLFECTIN™ (Thermo Fisher Scientific), OLIGOFECTAMINE™ (Thermo Fisher Scientific), LIPOFECTACE™, FUGENE™ (Roche, Basel, Switzerland), FUGENE™ HD (Roche), TRANSFECTAM™ (Transfectam, Promega, Madison, Wis.), TFX-10™ (Promega), TFX-20™ (Promega), TFX-50™ (Promega), TRANSFECTIN™ (BioRad, Hercules, Calif.), SILENTFECT™ (Bio-Rad), Effectene™ (Qiagen, Valencia, Calif.), DC-chol (Avanti Polar Lipids), GENEPORTER™ (Gene Therapy Systems, San Diego, Calif.), DHARMAFECT 1™ (Dharmacon, Lafayette, Colo.), DHARMAFECT 2™ (Dharmacon), DHARMAFECT 3™ (Dharmacon), DHARMAFECT 4™ (Dharmacon), ESCORT™ III (Sigma, St. Louis, Mo.), and ESCORT™ IV (Sigma Chemical Co.). Nucleic acids, such as the ssDNA molecule or the dsDNA construct, can also be delivered to a cell via microfluidics methods known to those of skill in the art.

Methods of non-viral delivery of nucleic acids in vivo or ex vivo include electroporation, lipofection (see, U.S. Pat. Nos. 5,049,386; 4,946,787 and commercially available reagents such as Transfectam™ and Lipofectin™), microinjection, biolistics, virosomes, liposomes (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787), immunoliposomes, polycation or lipid: nucleic acid conjugates, naked DNA, and agent-enhanced uptake of DNA. Sonoporation using, e.g., the Sonitron 2000 system (Rich-Mar) can also be used for delivery of nucleic acids.

A single-stranded DNA (ssDNA) molecule described herein can also be administered directly to an organism for transduction of cells in vivo. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

Methods for introduction of a single-stranded DNA (ssDNA) molecule can be delivered into hematopoietic stem cells, for example, by the methods as described, for example, in U.S. Pat. No. 5,928,638.

Delivery reagents such as liposomes, nanocapsules, microparticles, microspheres, lipid particles, vesicles, and the like, can be used for the introduction of the compositions of the present disclosure into suitable host cells. In particular, the nucleic acids can be formulated for delivery either encapsulated in a lipid particle, a liposome, a vesicle, a nanosphere, a nanoparticle, a gold particle, or the like. Such formulations can be preferred for the introduction of pharmaceutically acceptable formulations of the nucleic acids disclosed herein.

Various delivery methods known in the art or modifications thereof can be used to deliver a single-stranded DNA (ssDNA) molecule described herein in vitro or in vivo. For example, in some embodiments, single-stranded DNA (ssDNA) molecules are delivered by making transient penetration in cell membrane by mechanical, electrical, ultrasonic, hydrodynamic, or laser-based energy so that DNA entrance into the targeted cells is facilitated. For example, a single-stranded DNA (ssDNA) molecule can be delivered by transiently disrupting cell membrane by squeezing the cell through a size-restricted channel or by other means known in the art. In some cases, a single-stranded DNA (ssDNA) molecule alone is directly injected as naked DNA into skin, thymus, cardiac muscle, skeletal muscle, or liver cells. In some cases, a single-stranded DNA (ssDNA) molecule is delivered by gene gun. Gold or tungsten spherical particles (1-3 μm diameter) coated with capsid-free AAV vectors can be accelerated to high speed by pressurized gas to penetrate into target tissue cells.

In some embodiments, electroporation is used to deliver a closed-ended DNA vector, including a single-stranded DNA (ssDNA) molecule. Electroporation causes temporary destabilization of the cell membrane target cell tissue by insertion of a pair of electrodes into the tissue so that DNA molecules in the surrounding media of the destabilized membrane would be able to penetrate into cytoplasm and nucleoplasm of the cell. Electroporation has been used in vivo for many types of tissues, such as skin, lung, and muscle.

In some cases, a single-stranded DNA (ssDNA) molecule, is delivered by hydrodynamic injection, which is a simple and highly efficient method for direct intracellular delivery of any water-soluble compounds and particles into internal organs and skeletal muscle in an entire limb.

In some cases, a single-stranded DNA (ssDNA) molecule is delivered by ultrasound by making nanoscopic pores in membrane to facilitate intracellular delivery of DNA particles into cells of internal organs or tumors, so the size and concentration of plasmid DNA have great role in efficiency of the system. In some cases, single-stranded DNA (ssDNA) molecules are delivered by magnetofection by using magnetic fields to concentrate particles containing nucleic acid into the target cells.

In some cases, chemical delivery systems can be used, for example, by using nanomeric complexes, which include compaction of negatively charged nucleic acid by polycationic nanomeric particles, belonging to cationic liposome/micelle or cationic polymers. Cationic lipids used for the delivery method includes, but not limited to monovalent cationic lipids, polyvalent cationic lipids, guanidine containing compounds, cholesterol derivative compounds, cationic polymers, (e.g., poly (ethylenimine), poly-L-lysine, protamine, other cationic polymers), and lipid-polymer hybrid.

Compositions comprising a single-stranded DNA (ssDNA) molecule described herein and a pharmaceutically acceptable carrier are specifically contemplated herein. In some embodiments, the single-stranded DNA (ssDNA) molecule is formulated with a lipid delivery system, for example, liposomes as described herein. In some embodiments, such compositions are administered by any route desired by a skilled practitioner. The compositions may be administered to a subject by different routes including orally, parenterally, sublingually, transdermally, rectally, transmucosally, topically, via inhalation, via buccal administration, intrapleurally, intravenous, intra-arterial, intraperitoneal, subcutaneous, intramuscular, intranasal intrathecal, and intraarticular or combinations thereof. For veterinary use, the composition may be administered as a suitably acceptable formulation in accordance with normal veterinary practice. The veterinarian may readily determine the dosing regimen and route of administration that is most appropriate for a particular animal. The compositions may be administered by traditional syringes, needleless injection devices, “microprojectile bombardment gene guns”, or other physical methods such as electroporation (“EP”), hydrodynamic methods or ultrasound.

In some cases, a single-stranded DNA (ssDNA) molecule is delivered by hydrodynamic injection, which is a simple and highly efficient method for direct intracellular delivery of any water-soluble compounds and particles into internal organs and skeletal muscle in an entire limb.

In some cases, a single-stranded DNA (ssDNA) molecule is delivered by ultrasound by making nanoscopic pores in membrane to facilitate intracellular delivery of DNA particles into cells of internal organs or tumors, so the size and concentration of the ssDNA molecule have a great role in efficiency of the system. In some cases, single-stranded DNA (ssDNA) molecules are delivered by magnetofection by using magnetic fields to concentrate particles containing nucleic acid into the target cells.

In some cases, chemical delivery systems can be used, for example, by using nanomeric complexes, which include compaction of negatively charged nucleic acid by polycationic nanomeric particles, belonging to cationic liposome/micelle or cationic polymers. Cationic lipids used for the delivery method includes, but not limited to monovalent cationic lipids, polyvalent cationic lipids, guanidine containing compounds, cholesterol derivative compounds, cationic polymers, (e.g., poly (ethylenimine), poly-L-lysine, protamine, other cationic polymers), and lipid-polymer hybrid.

### A. Exosomes

In some embodiments, a single-stranded DNA (ssDNA) molecule described herein is delivered by being packaged in an exosome. Exosomes are small membrane vesicles of endocytic origin that are released into the extracellular environment following fusion of multivesicular bodies with the plasma membrane. Their surface consists of a lipid bilayer from the donor cell's cell membrane, they contain cytosol from the cell that produced the exosome, and exhibit membrane proteins from the parental cell on the surface. Exosomes are produced by various cell types including epithelial cells, B and T lymphocytes, mast cells (MC) as well as dendritic cells (DC). Some embodiments, exosomes with a diameter between 10 nm and 1 μm, between 20 nm and 500 nm, between 30 nm and 250 nm, between 50 nm and 100 nm are envisioned for use. Exosomes can be isolated for delivery to target cells using either their donor cells or by introducing specific nucleic acids into them. Various approaches known in the art can be used to produce exosomes containing capsid-free vectors of the present disclosure.

### B. Microparticle/Nanoparticles

In some aspects, the disclosure provides for a lipid nanoparticle comprising a DNA vector, including a single-stranded DNA (ssDNA) molecule described herein and an ionizable lipid. For example, a lipid nanoparticle formulation that is made and loaded with synthetic AAV obtained by the process as disclosed in International Application PCT/US2018/050042, filed on Sep. 7, 2018 (published as International patent publication No. WO 2019/051289 A1), which is incorporated herein. This can be accomplished by high energy mixing of ethanolic lipids with aqueous synthetic AAV at low pH which protonates the ionizable lipid and provides favorable energetics for synthetic AAV/lipid association and nucleation of particles. The particles can be further stabilized through aqueous dilution and removal of the organic solvent. The particles can be concentrated to the desired level.

Generally, the lipid particles are prepared at a total lipid to synthetic AAV (mass or weight) ratio of from about 10:1 to 30:1. In some embodiments, the lipid to ssDNA molecule or the dsDNA construct ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of lipids and synthetic AAV can be adjusted to provide a desired N/P ratio, for example, N/P ratio of 3, 4, 5, 6, 7, 8, 9, 10 or higher. Generally, the lipid particle formulation's overall lipid content can range from about 5 mg/ml to about 30 mg/mL.

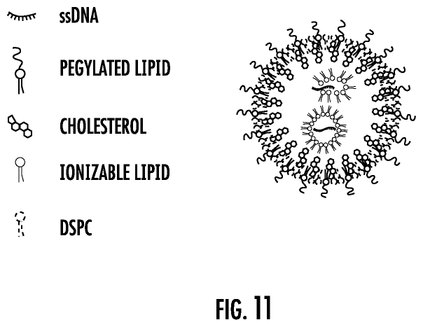

An exemplary lipid nanoparticle (LNP) formulation encapsulating a ssDNA molecule as described herein is depicted in FIG. 11.

The ionizable lipid is typically employed to condense the nucleic acid cargo, e.g., ssDNA as described herein at low pH and to drive membrane association and fusogenicity. Generally, ionizable lipids are lipids comprising at least one amino group that is positively charged or becomes protonated under acidic conditions, for example at pH of 6.5 or lower. Ionizable lipids are also referred to as cationic lipids herein.

Exemplary ionizable lipids are described in International PCT patent publications WO2015/095340, WO2015/199952, WO2018/011633, WO2017/049245, WO2015/061467, WO2012/040184, WO2012/000104, WO2015/074085, WO2016/081029, WO2017/004143, WO2017/075531, WO2017/117528, WO2011/022460, WO2013/148541, WO2013/116126, WO2011/153120, WO2012/044638, WO2012/054365, WO2011/090965, WO2013/016058, WO2012/162210, WO2008/042973, WO2010/129709, WO2010/144740, WO2012/099755, WO2013/049328, WO2013/086322, WO2013/086373, WO2011/071860, WO2009/132131, WO2010/048536, WO2010/088537, WO2010/054401, WO2010/054406, WO2010/054405, WO2010/054384, WO2012/016184, WO2009/086558, WO2010/042877, WO2011/000106, WO2011/000107, WO2005/120152, WO2011/141705, WO2013/126803, WO2006/007712, WO2011/038160, WO2005/121348, WO2011/066651, WO2009/127060, WO2011/141704, WO2006/069782, WO2012/031043, WO2013/006825, WO2013/033563, WO2013/089151, WO2017/099823, WO2015/095346, and WO2013/086354, and US patent publications US2016/0311759, US2015/0376115, US2016/0151284, US2017/0210697, US2015/0140070, US2013/0178541, US2013/0303587, US2015/0141678, US2015/0239926, US2016/0376224, US2017/0119904, US2012/0149894, US2015/0057373, US2013/0090372, US2013/0274523, US2013/0274504, US2013/0274504, US2009/0023673, US2012/0128760, US2010/0324120, US2014/0200257, US2015/0203446, US2018/0005363, US2014/0308304, US2013/0338210, US2012/0101148, US2012/0027796, US2012/0058144, US2013/0323269, US2011/0117125, US2011/0256175, US2012/0202871, US2011/0076335, US2006/0083780, US2013/0123338, US2015/0064242, US2006/0051405, US2013/0065939, US2006/0008910, US2003/0022649, US2010/0130588, US2013/0116307, US2010/0062967, US2013/0202684, US2014/0141070, US2014/0255472, US2014/0039032, US2018/0028664, US2016/0317458, and US2013/0195920, the contents of all of which are incorporated herein by reference in their entirety.

In some embodiments, the ionizable lipid is MC3 (6Z,9Z,28Z,31Z)-heptatriaconta-6,9,28,31-tetraen-19-yl-4-(dimethylamino)butanoate (DLin-MC3-DMA or MC3) having the following structure:

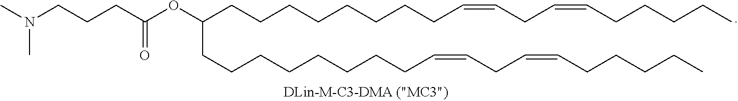 [SMILES: CCCCC/C=C\C/C=C\CCCCCCCCC(CCCCCCCC/C=C\C/C=C\CCCCC)OC(=O)CCCN(C)C]

The lipid DLin-MC3-DMA is described in Jayaraman et al., Angew. Chem. Int. Ed Engl. (2012), 51(34): 8529-8533, content of which is incorporated herein by reference in its entirety.

In some embodiments, the ionizable lipid is the lipid ATX-002 as described in WO2015/074085, content of which is incorporated herein by reference in its entirety.

In some embodiments, the ionizable lipid is (13Z,16Z)—N,N-dimethyl-3-nonyldocosa-13,16-dien-1-amine, as described in WO2012/040184, content of which is incorporated herein by reference in its entirety.

In some embodiments, the ionizable lipid is Compound 6 or Compound 22 as described in WO2015/199952, content of which is incorporated herein by reference in its entirety.

Without limitations, ionizable lipid can comprise 20-90% (mol) of the total lipid present in the lipid nanoparticle. For example, ionizable lipid molar content can be 20-70% (mol), 30-60% (mol) or 40-50% (mol) of the total lipid present in the lipid nanoparticle. In some embodiments, ionizable lipid comprises from about 50 mol % to about 90 mol % of the total lipid present in the lipid nanoparticle.

In some aspects, the lipid nanoparticle can further comprise a non-cationic lipid. Non-ionic lipids include amphipathic lipids, neutral lipids and anionic lipids. Accordingly, the non-cationic lipid can be a neutral uncharged, zwitterionic, or anionic lipid. Non-cationic lipids are typically employed to enhance fusogenicity.

Exemplary non-cationic lipids envisioned for use in the methods and compositions comprising a DNA vector, including a synthetic vector produced using the synthetic process as described herein are described in International Application PCT/US2018/050042, filed on Sep. 7, 2018 (published as International patent publication No. WO 2019/051289 A1), and PCT/US2018/064242, filed on Dec. 6, 2018 (published as International patent publication No. WO 2019/113310 A1), each of which is incorporated herein in its entirety.

Exemplary non-cationic lipids are described in International application Publication WO2017/099823 and US patent publication US2018/0028664, the contents of both of which are incorporated herein by reference in their entirety.

The non-cationic lipid can comprise 0-30% (mol) of the total lipid present in the lipid nanoparticle. For example, the non-cationic lipid content is 5-20% (mol) or 10-15% (mol) of the total lipid present in the lipid nanoparticle. In various embodiments, the molar ratio of ionizable lipid to the neutral lipid ranges from about 2:1 to about 8:1.

In some embodiments, the lipid nanoparticles do not comprise any phospholipids. In some aspects, the lipid nanoparticle can further comprise a component, such as a sterol, to provide membrane integrity.

One exemplary sterol that can be used in the lipid nanoparticle is cholesterol and derivatives thereof. Exemplary cholesterol derivatives are described in International application WO2009/127060 and US patent publication US2010/0130588, contents of both of which are incorporated herein by reference in their entirety.

The component providing membrane integrity, such as a sterol, can comprise 0-50% (mol) of the total lipid present in the lipid nanoparticle. In some embodiments, such a component is 20-50% (mol) 30-40% (mol) of the total lipid content of the lipid nanoparticle.

In some aspects, the lipid nanoparticle can further comprise a polyethylene glycol (PEG) or a conjugated lipid molecule. Generally, these are used to inhibit aggregation of lipid nanoparticles and/or provide steric stabilization. Exemplary conjugated lipids include, but are not limited to, PEG-lipid conjugates, polyoxazoline (POZ)-lipid conjugates, polyamide-lipid conjugates (such as ATTA-lipid conjugates), cationic-polymer lipid (CPL) conjugates, and mixtures thereof. In some embodiments, the conjugated lipid molecule is a PEG-lipid conjugate, for example, a (methoxy polyethylene glycol)-conjugated lipid. Exemplary PEG-lipid conjugates include, but are not limited to, PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a PEGylated phosphatidylethanoloamine (PEG-PE), PEG succinate diacylglycerol (PEGS-DAG) (such as 4-O-(2′,3′-di (tetradecanoyloxy) propyl-1-O-(w-methoxy (polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, or a mixture thereof. Additional exemplary PEG-lipid conjugates are described, for example, in U.S. Pat. Nos. 5,885,613, 6,287,591, US2003/0077829, US2003/0077829, US2005/0175682, US2008/0020058, US2011/0117125, US2010/0130588, US2016/0376224, and US2017/0119904, the contents of all of which are incorporated herein by reference in their entirety.

In some embodiments, a PEG-lipid is a compound disclosed in US2018/0028664, the content of which is incorporated herein by reference in its entirety.

In some embodiments, a PEG-lipid is disclosed in US20150376115 or in US2016/0376224, the content of both of which is incorporated herein by reference in its entirety.

The PEG-DAA conjugate can be, for example, PEG-dilauryloxypropyl, PEG-dimyristyloxypropyl, PEG-dipalmityloxypropyl, or PEG-distearyloxypropyl. The PEG-lipid can be one or more of PEG-DMG, PEG-dilaurylglycerol, PEG-dipalmitoylglycerol, PEG-disterylglycerol, PEG-dilaurylglycamide, PEG-dimyristylglycamide, PEG-dipalmitoylglycamide, PEG-disterylglycamide, PEG-cholesterol (1-[8′-(Cholest-5-en-3 [beta]-oxy) carboxamido-3′,6′-dioxaoctanyl] carbamoyl-[omega]-methyl-poly(ethylene glycol), PEG-DMB (3,4-Ditetradecoxylbenzyl-[omega]-methyl-poly(ethylene glycol) ether), and 1,2-dimyristoyl-sn-glycero-3-phosphoethanolamine-N-[methoxy(polyethylene glycol)-2000]. In some examples, the PEG-lipid can be selected from the group consisting of PEG-DMG, 1,2-dimyristoyl-sn-glycero-3-phosphoethanolamine-N-[methoxy(polyethylene glycol)-2000].

Lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (CPL) conjugates can be used in place of or in addition to the PEG-lipid. Exemplary conjugated lipids, i.e., PEG-lipids, (POZ)-lipid conjugates, ATTA-lipid conjugates and cationic polymer-lipids are described in the International patent application publications WO1996/010392, WO1998/051278, WO2002/087541, WO2005/026372, WO2008/147438, WO2009/086558, WO2012/000104, WO2017/117528, WO2017/099823, WO2015/199952, WO2017/004143, WO2015/095346, WO2012/000104, WO2012/000104, and WO2010/006282, US patent application publications US2003/0077829, US2005/0175682, US2008/0020058, US2011/0117125, US2013/0303587, US2018/0028664, US2015/0376115, US2016/0376224, US2016/0317458, US2013/0303587, US2013/0303587, and US20110123453, and US patents U.S. Pat. Nos. 5,885,613, 6,287,591, 6,320,017, and 6,586,559, the contents of all of which are incorporated herein by reference in their entirety.

In some embodiments, the one or more additional compounds can be a therapeutic agent. The therapeutic agent can be selected from any class suitable for the therapeutic objective. In other words, the therapeutic agent can be selected from any class suitable for the therapeutic objective. In other words, the therapeutic agent can be selected according to the treatment objective and biological action desired. For example, if the synthetic AAV within the LNP is useful for treating cancer, the additional compound can be an anti-cancer agent (e.g., a chemotherapeutic agent, a targeted cancer therapy (including, but not limited to, a small molecule, an antibody, or an antibody-drug conjugate). In another example, if the LNP containing the synthetic AAV is useful for treating an infection, the additional compound can be an antimicrobial agent (e.g., an antibiotic or antiviral compound). In yet another example, if the LNP containing the synthetic AAV is useful for treating an immune disease or disorder, the additional compound can be a compound that modulates an immune response (e.g., an immunosuppressant, immunostimulatory compound, or compound modulating one or more specific immune pathways). In some embodiments, different cocktails of different lipid nanoparticles containing different compounds, such as a synthetic AAV encoding a different protein or a different compound, such as a therapeutic may be used in the compositions and methods of the disclosure.

In some embodiments, the additional compound is an immune modulating agent. For example, the additional compound is an immunosuppressant. In some embodiments, the additional compound is an immune stimulatory agent.

Also provided herein is a pharmaceutical composition comprising the lipid nanoparticle-encapsulated synthetically produced single-stranded DNA (ssDNA) molecule described herein and a pharmaceutically acceptable carrier or excipient.

In some aspects, the disclosure provides for a lipid nanoparticle formulation further comprising one or more pharmaceutical excipients. In some embodiments, the lipid nanoparticle formulation further comprises sucrose, tris, trehalose and/or glycine.

A single-stranded DNA (ssDNA) molecule described herein can be complexed with the lipid portion of the particle or encapsulated in the lipid position of the lipid nanoparticle. In some embodiments, a DNA vector, including a single-stranded DNA (ssDNA) molecule can be fully encapsulated in the lipid position of the lipid nanoparticle, thereby protecting it from degradation by a nuclease, e.g., in an aqueous solution. In some embodiments, a DNA vector, including a single-stranded DNA (ssDNA) molecule in the lipid nanoparticle is not substantially degraded after exposure of the lipid nanoparticle to a nuclease at 37° C. for at least about 20, 30, 45, or 60 minutes. In some embodiments, the synthetic AAV in the lipid nanoparticle is not substantially degraded after incubation of the particle in serum at 37° C. for at least about 30, 45, or 60 minutes or at least about 2, 3, 4, 5, 6, 7, 8, 9, 10, 12, 14, 16, 18, 20, 22, 24, 26, 28, 30, 32, 34, or 36 hours.

In certain embodiments, the lipid nanoparticles are substantially non-toxic to a subject, e.g., to a mammal such as a human. In some aspects, the lipid nanoparticle formulation is a lyophilized powder.

In some embodiments, lipid nanoparticles are solid core particles that possess at least one lipid bilayer. In other embodiments, the lipid nanoparticles have a non-bilayer structure, i.e., a non-lamellar (i.e., non-bilayer) morphology. Without limitations, the non-bilayer morphology can include, for example, three dimensional tubes, rods, cubic symmetries, etc. For example, the morphology of the lipid nanoparticles (lamellar vs. non-lamellar) can readily be assessed and characterized using, e.g., Cryo-TEM analysis as described in US2010/0130588, the content of which is incorporated herein by reference in its entirety.

In some further embodiments, the lipid nanoparticles having a non-lamellar morphology are electron dense. In some aspects, the disclosure provides for a lipid nanoparticle that is either unilamellar or multilamellar in structure. In some aspects, the disclosure provides for a lipid nanoparticle formulation that comprises multi-vesicular particles and/or foam-based particles.

By controlling the composition and concentration of the lipid components, one can control the rate at which the lipid conjugate exchanges out of the lipid particle and, in turn, the rate at which the lipid nanoparticle becomes fusogenic. In addition, other variables including, e.g., pH, temperature, or ionic strength, can be used to vary and/or control the rate at which the lipid nanoparticle becomes fusogenic. Other methods which can be used to control the rate at which the lipid nanoparticle becomes fusogenic will be apparent to those of ordinary skill in the art based on this disclosure. It will also be apparent that by controlling the composition and concentration of the lipid conjugate, one can control the lipid particle size.

The pKa of formulated cationic lipids can be correlated with the effectiveness of the LNPs for delivery of nucleic acids (see Jayaraman et al., Angewandte Chemie, International Edition (2012), 51 (34), 8529-8533; Semple et al., Nature Biotechnology 28, 172-176 (2010), both of which are incorporated by reference in their entirety). The preferred range of pKa is ~5 to ~7. The pKa of the cationic lipid can be determined in lipid nanoparticles using an assay based on fluorescence of 2-(p-toluidino)-6-napthalene sulfonic acid (TNS).Generally, lipid nanoparticles comprise an ionizable amino lipid (e.g., heptatriaconta-6,9,28,31-tetraen-19-yl 4-(dimethylamino)butanoate, DLin-MC3-DMA, a phosphatidylcholine (1,2-distearoyl-sn-glycero-3-phosphocholine, DSPC), cholesterol and a coat lipid (polyethylene glycol-dimyristolglycerol, PEG-DMG), for example as disclosed by Tam et al. (2013). Advances in Lipid Nanoparticles for siRNA delivery. Pharmaceuticals 5 (3): 498-507.

In some embodiments, a lipid nanoparticle has a mean diameter between about 10 and about 1000 nm. In some embodiments, a lipid nanoparticle has a diameter that is less than 300 nm. In some embodiments, a lipid nanoparticle has a diameter between about 10 and about 300 nm. In some embodiments, a lipid nanoparticle has a diameter that is less than 200 nm. In some embodiments, a lipid nanoparticle has a diameter between about 25 and about 200 nm. In some embodiments, a lipid nanoparticle preparation (e.g., composition comprising a plurality of lipid nanoparticles) has a size distribution in which the mean size (e.g., diameter) is about 40 nm to about 200 nm, and more typically the mean size is about 100 nm or less (e.g., 100 nm, 90 nm, 85 nm 80 nm, 75 nm, 70 nm, 65 nm, 60 nm, 55 nm, 50 nm, and 45 nm in diameter).

Various lipid nanoparticles known in the art can be used to deliver a single-stranded DNA (ssDNA) molecule. For example, various delivery methods using lipid nanoparticles are described in U.S. Pat. Nos. 9,404,127, 9,006,417 and 9,518,272.

In some embodiments, a single-stranded DNA (ssDNA) molecule is delivered by a gold nanoparticle. Generally, a nucleic acid can be covalently bound to a gold nanoparticle or non-covalently bound to a gold nanoparticle (e.g., bound by a charge-charge interaction), for example as described by Ding et al. (2014). Gold Nanoparticles for Nucleic Acid Delivery. Mol. Ther. 22 (6); 1075-1083. In some embodiments, gold nanoparticle-nucleic acid conjugates are produced using methods described, for example, in U.S. Pat. No. 6,812,334.

In some embodiments, ssDNA molecules described herein can be readily formulated in high concentrations of chitosan-nucleic acid polyplex compositions and administered orally in DNA enteric coated pills described in U.S. Pat. Nos. 8,846,102; 9,404,088; and 9,850,323, each of which is incorporated herein by its entirety. In some embodiments, a lipid nanoparticle described herein is conjugated (e.g., covalently bound to an agent that increases cellular uptake. An “agent that increases cellular uptake” is a molecule that facilitates transport of a nucleic acid or a lipid nanoparticle across a lipid membrane. For example, a lipid nanoparticle can be conjugated to a cell penetrating peptide (CPP) (e.g., penetratin, TAT, Syn1B, etc.) and/or polyamines (e.g., spermine). Further examples of agents that increase cellular uptake are disclosed, for example, in Winkler (2013). Oligonucleotide conjugates for therapeutic applications. Ther. Deliv. 4 (7); 791-809.

In some embodiments, a lipid nanoparticle described herein is conjugated to a polymer (e.g., a polymeric molecule) or a folate molecule (e.g., folic acid molecule). Generally, delivery of nucleic acids, lipids, and lipid nanoparticles conjugated to polymers is known in the art, for example as described in WO2000/34343 and WO2008/022309. In some embodiments, a lipid and/or a lipid nanoparticle is conjugated to a poly (amide) polymer, for example as described by U.S. Pat. No. 8,987,377. In some embodiments, a lipid and/or a lipid nanoparticle described by the disclosure is conjugated to a folic acid molecule as described in U.S. Pat. No. 8,507,455.

In some embodiments, a lipid and/or a lipid nanoparticle is conjugated to a carbohydrate, for example as described in U.S. Pat. No. 8,450,467. In some embodiments, a lipid and/or a lipid nanoparticle is conjugated to GalNAc. In some embodiments, a lipid and/or a lipid nanoparticle is conjugated to an antibody, e.g., a single-chain antibody such as an scFv.

### C. Nanocapsules

Alternatively, nanocapsule formulations of a single-stranded DNA (ssDNA) molecule described herein can be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

### D. Liposomes

A single-stranded DNA (ssDNA) molecule described herein can be added to liposomes for delivery to a cell or target organ in a subject. Liposomes are vesicles that possess at least one lipid bilayer. Liposomes are typical used as carriers for drug/therapeutic delivery in the context of pharmaceutical development. They work by fusing with a cellular membrane and repositioning its lipid structure to deliver a drug or active pharmaceutical ingredient (API). Liposome compositions for such delivery are composed of phospholipids, especially compounds having a phosphatidylcholine group, however these compositions may also include other lipids.

The formation and use of liposomes is generally known to those of skill in the art. Liposomes have been developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

A single-stranded DNA (ssDNA) molecule described herein can be added to liposomes for delivery to a cell, e.g., a cell in need of expression of the transgene. Liposomes are vesicles that possess at least one lipid bilayer. Liposomes are typical used as carriers for drug/therapeutic delivery in the context of pharmaceutical development. They work by fusing with a cellular membrane and repositioning its lipid structure to deliver a drug or active pharmaceutical ingredient (API). Liposome compositions for such delivery are composed of phospholipids, especially compounds having a phosphatidylcholine group, however these compositions may also include other lipids.

In some aspects, the disclosure provides for a liposome formulation that includes one or more compounds with a polyethylene glycol (PEG) functional group (so-called “PEG-ylated compounds”) which can reduce the immunogenicity/antigenicity of, provide hydrophilicity and hydrophobicity to the compound(s) and reduce dosage frequency. Or the liposome formulation simply includes polyethylene glycol (PEG) polymer as an additional component. In such aspects, the molecular weight of the PEG or PEG functional group can be from 62 Da to about 5,000 Da.

In some aspects, the disclosure provides for a liposome formulation that will deliver an API with extended release or controlled release profile over a period of hours to weeks. In some related aspects, the liposome formulation may comprise aqueous chambers that are bound by lipid bilayers. In other related aspects, the liposome formulation encapsulates an API with components that undergo a physical transition at elevated temperature which releases the API over a period of hours to weeks.

In some aspects, the liposome formulation comprises sphingomyelin and one or more lipids disclosed herein. In some aspects, the liposome formulation comprises optisomes.

In some aspects, the disclosure provides for a liposome formulation that includes one or more lipids selected from: N-(carbonyl-methoxypolyethylene glycol 2000)-1,2-distearoyl-sn-glycero-3-phosphoethanolamine sodium salt, (distearoyl-sn-glycero-phosphoethanolamine), MPEG (methoxy polyethylene glycol)-conjugated lipid, HSPC (hydrogenated soy phosphatidylcholine); PEG (polyethylene glycol); DSPE (distearoyl-sn-glycero-phosphoethanolamine); DSPC (distearoylphosphatidylcholine); DOPC (dioleoylphosphatidylcholine); DPPG (dipalmitoylphosphatidylglycerol); EPC (egg phosphatidylcholine); DOPS (dioleoylphosphatidylserine); POPC (palmitoyloleoylphosphatidylcholine); SM (sphingomyelin); MPEG (methoxy polyethylene glycol); DMPC (dimyristoyl phosphatidylcholine); DMPG (dimyristoyl phosphatidylglycerol); DSPG (distearoylphosphatidylglycerol); DEPC (dierucoylphosphatidylcholine); DOPE (dioleoly-sn-glycero-phophoethanolamine). cholesteryl sulphate (CS), dipalmitoylphosphatidylglycerol (DPPG), DOPC (dioleoly-sn-glycero-phosphatidylcholine) or any combination thereof.

In some aspects, the disclosure provides for a liposome formulation comprising phospholipid, cholesterol and a PEG-ylated lipid in a molar ratio of 56:38:5. In some aspects, the liposome formulation's overall lipid content is from 2-16 mg/mL. In some aspects, the disclosure provides for a liposome formulation comprising a lipid containing a phosphatidylcholine functional group, a lipid containing an ethanolamine functional group and a PEG-ylated lipid. In some aspects, the disclosure provides for a liposome formulation comprising a lipid containing a phosphatidylcholine functional group, a lipid containing an ethanolamine functional group and a PEG-ylated lipid in a molar ratio of 3:0.015:2 respectively. In some aspects, the disclosure provides for a liposome formulation comprising a lipid containing a phosphatidylcholine functional group, cholesterol and a PEG-ylated lipid. In some aspects, the disclosure provides for a liposome formulation comprising a lipid containing a phosphatidylcholine functional group and cholesterol. In some aspects, the PEG-ylated lipid is PEG-2000-DSPE. In some aspects, the disclosure provides for a liposome formulation comprising DPPG, soy PC, MPEG-DSPE lipid conjugate and cholesterol.

In some aspects, the disclosure provides for a liposome formulation comprising one or more lipids containing a phosphatidylcholine functional group and one or more lipids containing an ethanolamine functional group. In some aspects, the disclosure provides for a liposome formulation comprising one or more: lipids containing a phosphatidylcholine functional group, lipids containing an ethanolamine functional group, and sterols, e.g., cholesterol. In some aspects, the liposome formulation comprises DOPC/DEPC; and DOPE.

In some aspects, the disclosure provides for a liposome formulation further comprising one or more pharmaceutical excipients, e.g., sucrose and/or glycine.

In some aspects, the disclosure provides for a liposome formulation that is either unilamellar or multilamellar in structure. In some aspects, the disclosure provides for a liposome formulation that comprises multi-vesicular particles and/or foam-based particles. In some aspects, the disclosure provides for a liposome formulation that are larger in relative size to common nanoparticles and about 150 to 250 nm in size. In some aspects, the liposome formulation is a lyophilized powder. In some aspects, the disclosure provides for a liposome formulation that is made and loaded with ssDNA molecule or the dsDNA construct disclosed or described herein, by adding a weak base to a mixture having the isolated ssDNA molecule or the dsDNA construct outside the liposome. This addition increases the pH outside the liposomes to approximately 7.3 and drives the API into the liposome. In some aspects, the disclosure provides for a liposome formulation having a pH that is acidic on the inside of the liposome. In such cases the inside of the liposome can be at pH 4-6.9, and more preferably pH 6.5. In other aspects, the disclosure provides for a liposome formulation made by using intra-liposomal drug stabilization technology. In such cases, polymeric or non-polymeric highly charged anions and intra-liposomal trapping agents are utilized, e.g., polyphosphate or sucrose octasulfate.

### VI. Kits

Any combination of the materials useful in the disclosed methods can be packaged together as a kit for performing any of the disclosed methods. Vectors and primers are useful components of such kits. Enzymes necessary for the disclosed methods can also be components of such kits. A skilled worker will recognize components of kits suitable for carrying out any of the methods of the invention. Optionally, the kits comprise instructions for performing the method. Kits of the invention may further comprise a support or matrix to which elements of the invention can be attached or immobilized. Other optional elements of a kit of the invention include suitable buffers, or the like, containers, or packaging materials. The reagents of the kit may be in containers in which the reagents are stable, e.g., in lyophilized form or stabilized liquids. The reagents may also be in single use form, e.g., in a form for a single amplification.

In one embodiment, the kit comprises, e.g., a set of primers, such as a set of primers as described herein.

### EXAMPLES

The following examples are provided by way of illustration not limitation. It will be appreciated by one of ordinary skill in the art that a DNA vector, including a ceDNA vector produced using the synthetic process as described herein can be constructed from any of the symmetric or asymmetric ITR configurations, comprising any of wild-type or modified ITRs as described herein, and that the following exemplary methods can be used to construct and assess the activity of such ceDNA vectors. While the methods are exemplified with certain ceDNA vectors, they are applicable to any DNA vector, including any ceDNA vector, in keeping with the description.

### Example 1. Exemplary Single-Stranded ceDNA ITRs

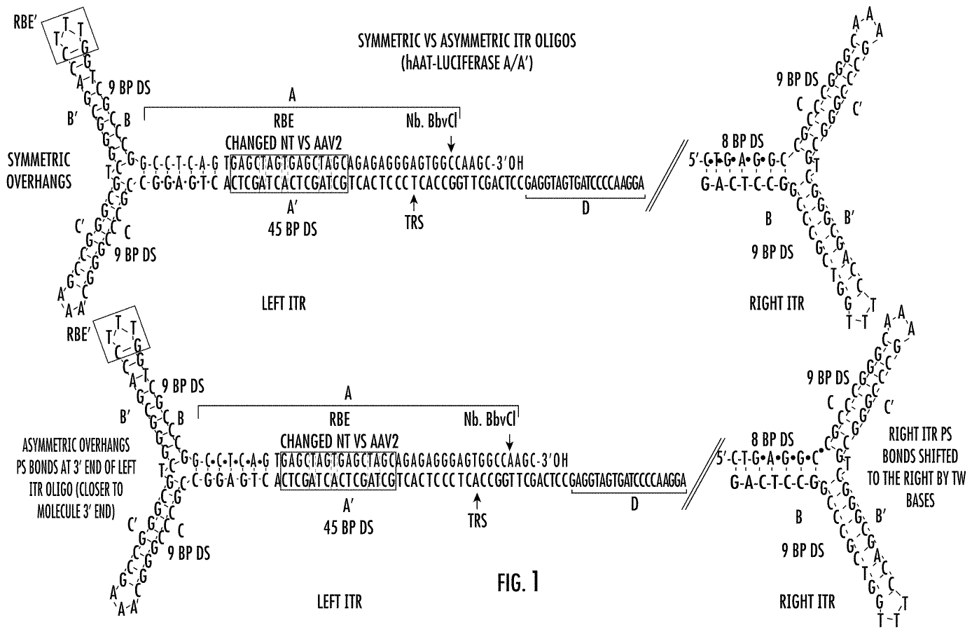

FIG. 1 depicts schematic drawings of symmetric versus asymmetric ITR oligos. The top drawing shows symmetric overhangs. The bottom drawing shows asymmetric overhangs, where the 3′ end of the left ITR has PS bonds (closer to the 3′ end of the molecule) and the right ITR has PS bonds shifted to the right by two bases.

Single-stranded DNA 013 (ss013) is an ssDNA comprising FVIII with PS bonds near the 5′ and 3′ ITR ends. The nucleic acid sequence of ss013 is set forth below as SEQ ID NO: 1.

SEQ ID NO: 1
GAGGCCGCCCGGGCAAAGCCCGGGCGTCGGGCGACCTTTGGTCGCCCGGCCTCAGTGAGCGAGCGAGC
GCGCAGAGAGGGAGTGGCCAACTCCATCACTAGGGGTTCCTCCTGCAGGTGTAGTTAATGATTAACCC
GCCATGCTACTTATCTACGTAGCCATGCTCTAGAGCCATAGAGCCCACCGCATCCCCAGCATGCCTGC
TATTGTCTTCCCAATCCTCCCCCTTGCTGTCCTGCCCCACCCCACCCCCCAGAATAGAATGACACCTA
CTCAGACAATGCGATGCAATTTCCTCATTTTATTAGGAAAGGACAGTGGGAGTGGCACCTTCCAGGGT
CAAGGAAGGCACGGGGGAGGGGCAAACAACAGATGGCTGGCAACTAGAAGGCACAGACAACACCACGG
AATTATCAGTGCCCAGCAACCTAGCCCCTGTCCAGCAGCGGGCAAGGCAGGCGGCGATGAGTTCTGCC
GTGGCGATCGGGAGGGGGAAAGCGAAAGTCCCAGAAAGGAGTTGACAGGTGGTGGCAATGCCCCAGCC
AGTGGGGGTTGCGTCAGCAAACACAGAGCACACCACGCCACGTTGACGGACAACGGGCCACAACTCCT
CTAAAAGAGACAGCAACCAGGATTTATACAAGGAGGAGAAAACGAAAGCCGTACGGGAAGCAATAGCT
AGATACAGAGGCTATAAAGCAGCATATCCACACAGCGTAAAAGGAGCAACATAGTTAAGAATATCAGT
CAATCTTTCACAAATTTTGTAATCCAGAGGTTGATTAACAGGAACAGAGCGTAAATAACGGGAAAGTT
TCTTAACATGTTTGTCTTGTGGCAATACACCTGAACTAGTAATTACATATCCCTAAAAATGTAAATGA
TTGCCCCACCATTTTGTTTTATTAACATTTAAATGTATACCCAAATCAAGAAAAACAGAACAAATATG
GGAATAAATGGCGGTAAGATGCTCTTAATTAATCAGTACAGGTCCTGGGCCTCACAGCCCAGCACCTC
CATCCTCAGGGCAATCTGGTGCACCCAGCTCTGGGGGTGAATCCTCAGGTATCTGGTCAGCAGGGGGG
GGTCCAGGCTGTTCACCACAGGGGTGAAGCTGTCCTGGTTGCCCTGGAACACCTTCACCTTGCCATTC
TGGAAGAACAGGGTCCACTGGTGGCCATCCTGGCTGCTGCTGATCAGGAACTCCTTCACATACATGCT
GGTCAGCAGGCTCTTCACCCCCTGGGTGGTCACCCCAGTCACCTTCATGGTCTTCTGGAAGTCCACCT
GCAGCCACTCCTTGGGGTTGTTGACCTGGGGCCTCCAGGCATTGCTCCTGCCCTGCAGGTGCAGCCTG
GCCTTGCTGGGGCTCCAGGTGGCAAACATGTTGGTGAAGTAGCTGCTGGCAGTGATCTGGGCATCAGA
GATGGCCTTGCTCTCCATGCCCAGGGGCATGCTGCAGCTGTTCAGGTCACAGCCCATCAGCTCCATCC
TCAGGGTGCTCCTGATGCTGTAGTGGGTGGGGTGCAGCCTGATGTATCTGGCAATGATGGGGGGGTTG
AAGATGTTGTGCTTGATGCCAGAGCTGTCCACATTGCCAAAGAACACCATCAGGGTGCCAGTGCTGTT
GCCCCTGTAGGTCTGCCACTTCTTGCCATCCAGGCTGTACATGATGATGAACTGGCTGATGTACAGGC
TGCTGAACTTCTGCCTGGCCCCCTGGGTCTTGATGCCATGGATGATCATGGGGGCCAGCAGGTCCACC
TTGATCCAGCTGAAGGGCTCCTTGGTGCTCCAGGCATTGATGCTGCCAGAGTAGTGCAGCCTGGCCAG
CTTGGGGGCCCACTGGCCATACTGGCCAGAGGCAGTGATCTGGAAGTCCCTGATGTGGCCAGAGGCCA
TGCCCAGGGGGGTCTGGCACTTGTTGCTGTACACCAGGAACAGGGTGCTCATGCCAGCATGCAGGTGC
TCCCCAATCAGGCACTCCACCCTCCAGATGCCAGCCTTGCTGGGCAGCATCTCCACAGTCTCAAACAC
CCCAGGGTACAGGTTGTACAGGGCCATCTTGTACTCCTCCTTCTTCCTCACAGTGAACACATGGCCAG
AGAAGTGGATGCTGTGGATGTTCTCATTGCTGCCCATGCTCAGCAGGTACCACCTGATCCTCTGGTCC
TGGGCCATCACCAGGCCAGGCAGGGTGTCCATGATGTAGCCATTGATGGCATGGAACCTGTAGTTCTC
CTTGAAGGTGGGGTCCTCCATCTGGATGTTGCAGGGGGCCCTGCAGTTCCTCTCCATGTTCTCAGTGA
AGTACCAGCTCTTGGTTTCATCAAAGATGGTGAAGAACAGGGCAAACTCCTGCACAGTCACCTGCCTG
CCATGGGCAGGGTTCAGGGTGTTGGTGTGGCACACCAGCAGGGGGCCAATCAGGCCAGAGTGCACATC
CTTCTCCAGGTCCACATCAGAGAAGTAGGCCCAGGCCTTGCAGTCAAACTCATCCTTGGTGGGGGCCA
TGTGGTGCTGCACCTTCCAGAAGTAGGTCTTGGTTTCATTGGGCTTCACAAAGTTCTTCCTGGGCTCA
GCCCCCTGCCTCTGGTCCTCCTCATAGCTGATCAGGCTGCTGTAGAAGCTGTAGGGCCTGCTGGCCTG
GTTCCTGAAGGTCACCATGATGTTGTCCTCCACCTCAGCCCTGATGTAGGGGCCCAGCAGGCCCAGGT
GCTCATTCAGCTCCCCTCTGTACAGGGGCTGGGTGAAGCTGCCATCAGTGAACTCCTGGAACACCACC
TTCTTGAACTGGGGCACAGAGCCAGACTGGGCCCTGTTCCTCAGCACATGGGGGCTGCTGCTCATGCC
ATAGTCCCACAGCCTCTCCACAGCAGCAATGAAGTAGTGCCTGGTCTTCTTCTGGAAGCTCCTGGGGC
TCTGGTTCTCGTCCTCGTCGTAGATGTCAAAGTCCTCCTTCTTCATCTCCACAGAGATGGTGTCATCA
TAGTCAATCTCCTCCTGGTCAGACTGCAGGGTGGTATTCTCTGGGATGGTGGTGGCATTGAACTGCTT
CTGCCTGGTGCTGGGGTGCCTGCTATTCTGGCTGAAGCTCCTGGGCTCAATGGCATTGTTCTTGCTCA
GCAGGTAGGCAGAGATGTCCTCATAGCTGTCCTCATAGTAGTCCCCAGTGTTCTTGTCACAGCTGGAG
ACTTTCAGCAGGGCAGTCATGCCCCTGTTCCTGAAGTCAGAGTTGTGGCAGCCCAGAATCCACAGGCC
AGGGTTCTCCATGCTCATGAACACAGTCTCCCCAGAGAAGGGGAACAGGGTCAGGGTGTCCTCATACA
CCATCTTGTGCTTGAAGGTGTAGCCAGAGAAGAACACAGACAGGAAGTCAGTCTGGGCCCCAATGCTC
AGGATGTACCAGTAGGCCACCTCATGCAGGCACACAGACAGCTGCAGGCTGTCAAACACATAGCCATT
GATGCTGTGCATGATGTTGCTGGCCTGGAACTCAGGGTCCTCCAGCTGCACCCCAGCAGGGTTGGGCA
GGAACCTCTGGATGTTCTCAGTCAGGTACCAGCTCCTGTTCTCATCAAACACAGAGAACAGGATCACA
TTCCTCTTGTCAGACATGATCTGGTTGCCCCTCTGGTCCACAGACTCCTTGTAGCAGATCAGCAGGGG
GCCAATCAGGCCAGAGGCCAGGTCCCTCTCCATGTTCACAAAGCTGCTGTAGTATCTGGTCAGGCACC
TGGGGTCAGACTTGGTGGGGCCATCCTCCACAGTCACAGTCCACTTGTACTTGAAGATCTCCCCAGGC
AGGATGGGGAAGTCCTTCAGGTGCTTCACCCCCTTGGGCAGCCTCCTGCTGTACAGGGGCCTCACATC
AGTGATGCCATGGGGGTAGATGTTGTAGGGCCTGCTGGCCTGGTTCTTGAAGATGATCAGCAGGGTGT
CCCCCACCTCCCCATACAGCAGGGGGCCCAGGATGCCAGACTCATGCTGGATGGCCTCCCTGGTCTTG
AAGGTTTCATCAGTGTAGGCCATGAACCTGACCTTCTTGTACTTCCTGCCAATCCTCTGGGGGCCATT
GTTCAGGTACTGGCTCTTGTAGCTCCTGTCATCAGGGGCCAGCACCAGGGGGGCATAGTCCCAGTCCT
CCTCCTCAGCAGCAATGTAGTGCACCCAGGTCTTGGGGTGCTTCTTGGCCACAGACCTGATCTGGATG
AAGCTGGGGCTGTTGTCATCATCAAACCTCACCACATCCATCTCAGAGTCAGTCAGGTCATCATCATA
GTCCTCAGCCTCCTCATTGTTCTTCATCCTCAGCTGGGGCTCCTCAGGGCAGCTGTCCACCTTCACAT
AGGCCTCCATGCCATCATGCTGGTGGCTGCTGATGTGGCAGAACAGCAGGAACTGGCCCAGGTCCATC
AGCAGGGTCTGGGCAGTCAGGAAGGTGATGGGGCTGATCTCCAGGCTGGCCTGCCTGTGGTTCCTGAC
CAGGAAGGTGTGGCCCTCCAGGAAGATGCTGTGCACCTCAGGGGTGGTGCCCATGCCAATCACATGCC
AGTACACAGACTTCCTGTGGCAGCCAATCAGGCCAGGCAGGCTCCTGTTCACATAGCCATTCACAGTG
TGCATCTTGGGCCAGGCCCTGGCAGAGGCAGCATCCCTGTCCTGCATCAGGCTGTTCTTGGTTTCAGA
GTGCCAGCTCTTGCCCTCATCAAACACAGCAAACAGCAGGATGAACTTGTGCAGGGTCTGGGTCTTCT
CCTTGGCCAGGCTGCCCTCCCTGCACACCAGCAGGGCCCCAATCAGGCCAGAGTTCAGGTCCTTCACC
AGGTCCACATGGCTCAGGTAGCTGTAGGTCAGGCACAGGGGGTCAGAGGCCATGGGGCCATTCTCCTT
CAGCACCTGCCACACATAGGTGTGGCTGCCCCCAGGGAACACCTTGTCATCCTCCTTCTCCCTCTGGC
TGGTCTGGTCATCATACTCAGCCCCCTCAGAGGCCTTCCAGTAGCTCACCCCCACAGCATGCAGGCTC
ACAGGGTGGCTGGCCATGTTCTTCAGGGTGATCACCACAGTGTCATACACCTCAGCCTGGATGGTGGG
GCCCAGCAGGCCCATCCAGGGGGGCCTGGGCTTGGCAATGTTGAACAGGTGGTCAGTGAACTCCACAA
ACAGGGTCTTCTTGTACACCACAGAGGTGTTGAAGGGGAAGCTCTTGGGCACTCTGGGGGGGAACCTG
GCATCCACAGGCAGCTCCCCCAGGTCAGACTGCATGTAGTCCCAGCTCAGCTCCACAGCCCCCAGGTA
GTATCTCCTGGTGGCAGAGAAGCAGAACCTCAGCAGGCACAGGAAGAAGCAGGTGCTCAGCTCAATCT
GCATGGTGGCGGCGTTTAAACCAACCTGAAAAAAAGTGATTTCAGGCAGGTGCTCCAGGTAATTAAAC
ATTAATACCCCACCAACCAACCATCCCTTAAACCCTTACCTCTTCAGGAGCTTGTGGATCTGTGTGAC
GGCTTCTCCTGGTGAAGGGGCTTTTATACCCCCTCCTTCCAACCCAGGCTGCTGATCCCTGCCAAGCT
GACTCCAAACCTGCTGATTCTGATTATTGACTTAGTCAACAAAAGGAGAATAAGTAACCTACACAAAT
ATGAACCTTGCCTAGGGAGATTAGAGTATCGGAACACTCGCTCTACGAAATGTGCAGACAGACGGGGA
TCCTCTAGAGCTACCTGCTGATCGCCCGGCCCCTGTTCAAACATGTCCTAATACTCTGTCTCTGCAAG
GGTCATCAGTAGTTTTCCATCTTACTCAACATCCTCCCAGTGGGTACCGTGGACTTAGCCCCTGTTTG
CTCCTCCGATAACTGGGGTGACCTTGGTTAATATTCACCAGCAGCCTCCCCCGGTGGACTTAGCCCCT
GTTTGCTCCTCCGATAACTGGGGTGACCTTGGTTAATATTCACCAGCAGCCTCCCCCGGTGGACTTAG
CCCCTGTTTGCTCCTCCGATAACTGGGGTGACCTTGGTTAATATTCACCAGCAGCCTCCCCCGCGGCC
GCGATAAGTAGCATGGCGGGTTAATCATTAACTACAAGGAACCCCTAGTGATGGAGCCTCAGCTTGGC
CACTCCCTCTCTGCGCGCTCGCTCGCTCACTGAGGCCGCCCGGGCAAAGCCCGGGCGTCGGGCGACCT
TTGGTCGCCCGGCCTCAGTGAGCGAGCGAGCGCGCAGAGAGGGAGTGGCCAAGC

Single-stranded DNA 041 (ss041) has the same sequence and PS bond locations as ss013, but was derived from synthetic double-stranded ceDNA generated from RCA KAN plasmid. The nucleic acid sequence of ss041 is set forth below as SEQ ID NO: 2.

SEQ ID NO: 2
GAGGCCGCCCGGGCAAAGCCCGGGCGTCGGGCGACCTTTGGTCGCCCGGCCTCAGTGAGCGAGCGAGC
GCGCAGAGAGGGAGTGGCCAACTCCATCACTAGGGGTTCCTCCTGCAGGTGTAGTTAATGATTAACCC
GCCATGCTACTTATCTACGTAGCCATGCTCTAGAGCCATAGAGCCCACCGCATCCCCAGCATGCCTGC
TATTGTCTTCCCAATCCTCCCCCTTGCTGTCCTGCCCCACCCCACCCCCCAGAATAGAATGACACCTA
CTCAGACAATGCGATGCAATTTCCTCATTTTATTAGGAAAGGACAGTGGGAGTGGCACCTTCCAGGGT
CAAGGAAGGCACGGGGGAGGGGCAAACAACAGATGGCTGGCAACTAGAAGGCACAGACAACACCACGG
AATTATCAGTGCCCAGCAACCTAGCCCCTGTCCAGCAGCGGGCAAGGCAGGCGGCGATGAGTTCTGCC
GTGGCGATCGGGAGGGGGAAAGCGAAAGTCCCAGAAAGGAGTTGACAGGTGGTGGCAATGCCCCAGCC
AGTGGGGGTTGCGTCAGCAAACACAGAGCACACCACGCCACGTTGACGGACAACGGGCCACAACTCCT
CTAAAAGAGACAGCAACCAGGATTTATACAAGGAGGAGAAAACGAAAGCCGTACGGGAAGCAATAGCT
AGATACAGAGGCTATAAAGCAGCATATCCACACAGCGTAAAAGGAGCAACATAGTTAAGAATATCAGT
CAATCTTTCACAAATTTTGTAATCCAGAGGTTGATTAACAGGAACAGAGCGTAAATAACGGGAAAGTT
TCTTAACATGTTTGTCTTGTGGCAATACACCTGAACTAGTAATTACATATCCCTAAAAATGTAAATGA
TTGCCCCACCATTTTGTTTTATTAACATTTAAATGTATACCCAAATCAAGAAAAACAGAACAAATATG
GGAATAAATGGCGGTAAGATGCTCTTAATTAATCAGTACAGGTCCTGGGCCTCACAGCCCAGCACCTC
CATCCTCAGGGCAATCTGGTGCACCCAGCTCTGGGGGTGAATCCTCAGGTATCTGGTCAGCAGGGGGG
GGTCCAGGCTGTTCACCACAGGGGTGAAGCTGTCCTGGTTGCCCTGGAACACCTTCACCTTGCCATTC
TGGAAGAACAGGGTCCACTGGTGGCCATCCTGGCTGCTGCTGATCAGGAACTCCTTCACATACATGCT
GGTCAGCAGGCTCTTCACCCCCTGGGTGGTCACCCCAGTCACCTTCATGGTCTTCTGGAAGTCCACCT
GCAGCCACTCCTTGGGGTTGTTGACCTGGGGCCTCCAGGCATTGCTCCTGCCCTGCAGGTGCAGCCTG
GCCTTGCTGGGGCTCCAGGTGGCAAACATGTTGGTGAAGTAGCTGCTGGCAGTGATCTGGGCATCAGA
GATGGCCTTGCTCTCCATGCCCAGGGGCATGCTGCAGCTGTTCAGGTCACAGCCCATCAGCTCCATCC
TCAGGGTGCTCCTGATGCTGTAGTGGGTGGGGTGCAGCCTGATGTATCTGGCAATGATGGGGGGGTTG
AAGATGTTGTGCTTGATGCCAGAGCTGTCCACATTGCCAAAGAACACCATCAGGGTGCCAGTGCTGTT
GCCCCTGTAGGTCTGCCACTTCTTGCCATCCAGGCTGTACATGATGATGAACTGGCTGATGTACAGGC
TGCTGAACTTCTGCCTGGCCCCCTGGGTCTTGATGCCATGGATGATCATGGGGGCCAGCAGGTCCACC
TTGATCCAGCTGAAGGGCTCCTTGGTGCTCCAGGCATTGATGCTGCCAGAGTAGTGCAGCCTGGCCAG
CTTGGGGGCCCACTGGCCATACTGGCCAGAGGCAGTGATCTGGAAGTCCCTGATGTGGCCAGAGGCCA
TGCCCAGGGGGGTCTGGCACTTGTTGCTGTACACCAGGAACAGGGTGCTCATGCCAGCATGCAGGTGC
TCCCCAATCAGGCACTCCACCCTCCAGATGCCAGCCTTGCTGGGCAGCATCTCCACAGTCTCAAACAC
CCCAGGGTACAGGTTGTACAGGGCCATCTTGTACTCCTCCTTCTTCCTCACAGTGAACACATGGCCAG
AGAAGTGGATGCTGTGGATGTTCTCATTGCTGCCCATGCTCAGCAGGTACCACCTGATCCTCTGGTCC
TGGGCCATCACCAGGCCAGGCAGGGTGTCCATGATGTAGCCATTGATGGCATGGAACCTGTAGTTCTC
CTTGAAGGTGGGGTCCTCCATCTGGATGTTGCAGGGGGCCCTGCAGTTCCTCTCCATGTTCTCAGTGA
AGTACCAGCTCTTGGTTTCATCAAAGATGGTGAAGAACAGGGCAAACTCCTGCACAGTCACCTGCCTG
CCATGGGCAGGGTTCAGGGTGTTGGTGTGGCACACCAGCAGGGGGCCAATCAGGCCAGAGTGCACATC
CTTCTCCAGGTCCACATCAGAGAAGTAGGCCCAGGCCTTGCAGTCAAACTCATCCTTGGTGGGGGCCA
TGTGGTGCTGCACCTTCCAGAAGTAGGTCTTGGTTTCATTGGGCTTCACAAAGTTCTTCCTGGGCTCA
GCCCCCTGCCTCTGGTCCTCCTCATAGCTGATCAGGCTGCTGTAGAAGCTGTAGGGCCTGCTGGCCTG
GTTCCTGAAGGTCACCATGATGTTGTCCTCCACCTCAGCCCTGATGTAGGGGCCCAGCAGGCCCAGGT
GCTCATTCAGCTCCCCTCTGTACAGGGGCTGGGTGAAGCTGCCATCAGTGAACTCCTGGAACACCACC
TTCTTGAACTGGGGCACAGAGCCAGACTGGGCCCTGTTCCTCAGCACATGGGGGCTGCTGCTCATGCC
ATAGTCCCACAGCCTCTCCACAGCAGCAATGAAGTAGTGCCTGGTCTTCTTCTGGAAGCTCCTGGGGC
TCTGGTTCTCGTCCTCGTCGTAGATGTCAAAGTCCTCCTTCTTCATCTCCACAGAGATGGTGTCATCA
TAGTCAATCTCCTCCTGGTCAGACTGCAGGGTGGTATTCTCTGGGATGGTGGTGGCATTGAACTGCTT
CTGCCTGGTGCTGGGGTGCCTGCTATTCTGGCTGAAGCTCCTGGGCTCAATGGCATTGTTCTTGCTCA
GCAGGTAGGCAGAGATGTCCTCATAGCTGTCCTCATAGTAGTCCCCAGTGTTCTTGTCACAGCTGGAG
ACTTTCAGCAGGGCAGTCATGCCCCTGTTCCTGAAGTCAGAGTTGTGGCAGCCCAGAATCCACAGGCC
AGGGTTCTCCATGCTCATGAACACAGTCTCCCCAGAGAAGGGGAACAGGGTCAGGGTGTCCTCATACA
CCATCTTGTGCTTGAAGGTGTAGCCAGAGAAGAACACAGACAGGAAGTCAGTCTGGGCCCCAATGCTC
AGGATGTACCAGTAGGCCACCTCATGCAGGCACACAGACAGCTGCAGGCTGTCAAACACATAGCCATT
GATGCTGTGCATGATGTTGCTGGCCTGGAACTCAGGGTCCTCCAGCTGCACCCCAGCAGGGTTGGGCA
GGAACCTCTGGATGTTCTCAGTCAGGTACCAGCTCCTGTTCTCATCAAACACAGAGAACAGGATCACA
TTCCTCTTGTCAGACATGATCTGGTTGCCCCTCTGGTCCACAGACTCCTTGTAGCAGATCAGCAGGGG
GCCAATCAGGCCAGAGGCCAGGTCCCTCTCCATGTTCACAAAGCTGCTGTAGTATCTGGTCAGGCACC
TGGGGTCAGACTTGGTGGGGCCATCCTCCACAGTCACAGTCCACTTGTACTTGAAGATCTCCCCAGGC
AGGATGGGGAAGTCCTTCAGGTGCTTCACCCCCTTGGGCAGCCTCCTGCTGTACAGGGGCCTCACATC
AGTGATGCCATGGGGGTAGATGTTGTAGGGCCTGCTGGCCTGGTTCTTGAAGATGATCAGCAGGGTGT
CCCCCACCTCCCCATACAGCAGGGGGCCCAGGATGCCAGACTCATGCTGGATGGCCTCCCTGGTCTTG
AAGGTTTCATCAGTGTAGGCCATGAACCTGACCTTCTTGTACTTCCTGCCAATCCTCTGGGGGCCATT
GTTCAGGTACTGGCTCTTGTAGCTCCTGTCATCAGGGGCCAGCACCAGGGGGGCATAGTCCCAGTCCT
CCTCCTCAGCAGCAATGTAGTGCACCCAGGTCTTGGGGTGCTTCTTGGCCACAGACCTGATCTGGATG
AAGCTGGGGCTGTTGTCATCATCAAACCTCACCACATCCATCTCAGAGTCAGTCAGGTCATCATCATA
GTCCTCAGCCTCCTCATTGTTCTTCATCCTCAGCTGGGGCTCCTCAGGGCAGCTGTCCACCTTCACAT
AGGCCTCCATGCCATCATGCTGGTGGCTGCTGATGTGGCAGAACAGCAGGAACTGGCCCAGGTCCATC
AGCAGGGTCTGGGCAGTCAGGAAGGTGATGGGGCTGATCTCCAGGCTGGCCTGCCTGTGGTTCCTGAC
CAGGAAGGTGTGGCCCTCCAGGAAGATGCTGTGCACCTCAGGGGTGGTGCCCATGCCAATCACATGCC
AGTACACAGACTTCCTGTGGCAGCCAATCAGGCCAGGCAGGCTCCTGTTCACATAGCCATTCACAGTG
TGCATCTTGGGCCAGGCCCTGGCAGAGGCAGCATCCCTGTCCTGCATCAGGCTGTTCTTGGTTTCAGA
GTGCCAGCTCTTGCCCTCATCAAACACAGCAAACAGCAGGATGAACTTGTGCAGGGTCTGGGTCTTCT
CCTTGGCCAGGCTGCCCTCCCTGCACACCAGCAGGGCCCCAATCAGGCCAGAGTTCAGGTCCTTCACC
AGGTCCACATGGCTCAGGTAGCTGTAGGTCAGGCACAGGGGGTCAGAGGCCATGGGGCCATTCTCCTT
CAGCACCTGCCACACATAGGTGTGGCTGCCCCCAGGGAACACCTTGTCATCCTCCTTCTCCCTCTGGC
TGGTCTGGTCATCATACTCAGCCCCCTCAGAGGCCTTCCAGTAGCTCACCCCCACAGCATGCAGGCTC
ACAGGGTGGCTGGCCATGTTCTTCAGGGTGATCACCACAGTGTCATACACCTCAGCCTGGATGGTGGG
GCCCAGCAGGCCCATCCAGGGGGGCCTGGGCTTGGCAATGTTGAACAGGTGGTCAGTGAACTCCACAA
ACAGGGTCTTCTTGTACACCACAGAGGTGTTGAAGGGGAAGCTCTTGGGCACTCTGGGGGGGAACCTG
GCATCCACAGGCAGCTCCCCCAGGTCAGACTGCATGTAGTCCCAGCTCAGCTCCACAGCCCCCAGGTA
GTATCTCCTGGTGGCAGAGAAGCAGAACCTCAGCAGGCACAGGAAGAAGCAGGTGCTCAGCTCAATCT
GCATGGTGGCGGCGTTTAAACCAACCTGAAAAAAAGTGATTTCAGGCAGGTGCTCCAGGTAATTAAAC
ATTAATACCCCACCAACCAACCATCCCTTAAACCCTTACCTCTTCAGGAGCTTGTGGATCTGTGTGAC
GGCTTCTCCTGGTGAAGGGGCTTTTATACCCCCTCCTTCCAACCCAGGCTGCTGATCCCTGCCAAGCT
GACTCCAAACCTGCTGATTCTGATTATTGACTTAGTCAACAAAAGGAGAATAAGTAACCTACACAAAT
ATGAACCTTGCCTAGGGAGATTAGAGTATCGGAACACTCGCTCTACGAAATGTGCAGACAGACGGGGA
TCCTCTAGAGCTACCTGCTGATCGCCCGGCCCCTGTTCAAACATGTCCTAATACTCTGTCTCTGCAAG
GGTCATCAGTAGTTTTCCATCTTACTCAACATCCTCCCAGTGGGTACCGTGGACTTAGCCCCTGTTTG
CTCCTCCGATAACTGGGGTGACCTTGGTTAATATTCACCAGCAGCCTCCCCCGGTGGACTTAGCCCCT
GTTTGCTCCTCCGATAACTGGGGTGACCTTGGTTAATATTCACCAGCAGCCTCCCCCGGTGGACTTAG
CCCCTGTTTGCTCCTCCGATAACTGGGGTGACCTTGGTTAATATTCACCAGCAGCCTCCCCCGCGGCC
GCGATAAGTAGCATGGCGGGTTAATCATTAACTACAAGGAACCCCTAGTGATGGAGCCTCAGCTTGGC
CACTCCCTCTCTGCGCGCTCGCTCGCTCACTGAGGCCGCCCGGGCAAAGCCCGGGCGTCGGGCGACCT
TTGGTCGCCCGGCCTCAGTGAGCGAGCGAGCGCGCAGAGAGGGAGTGGCCAAGC

Single-stranded DNA 042 (ss042) has no PS bonds on left ITR and PS bonds near 5′ end of right ITR generated by Endonuclease V nicking the inosine residue in left ITR of the synthetic double-stranded ceDNA generated from RCA KAN plasmid. The nucleic acid sequence of ss042 is set forth below as SEQ ID NO: 3.

SEQ ID NO: 3
GAGGCCGCCCGGGCAAAGCCCGGGCGTCGGGCGACCTTTGGTCGCCCGGCCTCAGTGAGCGAGCGAGC
GCGCAGAGAGGGAGTGGCCAACTCCATCACTAGGGGTTCCTCCTGCAGGTGTAGTTAATGATTAACCC
GCCATGCTACTTATCTACGTAGCCATGCTCTAGAGCCATAGAGCCCACCGCATCCCCAGCATGCCTGC
TATTGTCTTCCCAATCCTCCCCCTTGCTGTCCTGCCCCACCCCACCCCCCAGAATAGAATGACACCTA
CTCAGACAATGCGATGCAATTTCCTCATTTTATTAGGAAAGGACAGTGGGAGTGGCACCTTCCAGGGT
CAAGGAAGGCACGGGGGAGGGGCAAACAACAGATGGCTGGCAACTAGAAGGCACAGACAACACCACGG
AATTATCAGTGCCCAGCAACCTAGCCCCTGTCCAGCAGCGGGCAAGGCAGGCGGCGATGAGTTCTGCC
GTGGCGATCGGGAGGGGGAAAGCGAAAGTCCCAGAAAGGAGTTGACAGGTGGTGGCAATGCCCCAGCC
AGTGGGGGTTGCGTCAGCAAACACAGAGCACACCACGCCACGTTGACGGACAACGGGCCACAACTCCT
CTAAAAGAGACAGCAACCAGGATTTATACAAGGAGGAGAAAACGAAAGCCGTACGGGAAGCAATAGCT
AGATACAGAGGCTATAAAGCAGCATATCCACACAGCGTAAAAGGAGCAACATAGTTAAGAATATCAGT
CAATCTTTCACAAATTTTGTAATCCAGAGGTTGATTAACAGGAACAGAGCGTAAATAACGGGAAAGTT
TCTTAACATGTTTGTCTTGTGGCAATACACCTGAACTAGTAATTACATATCCCTAAAAATGTAAATGA
TTGCCCCACCATTTTGTTTTATTAACATTTAAATGTATACCCAAATCAAGAAAAACAGAACAAATATG
GGAATAAATGGCGGTAAGATGCTCTTAATTAATCAGTACAGGTCCTGGGCCTCACAGCCCAGCACCTC
CATCCTCAGGGCAATCTGGTGCACCCAGCTCTGGGGGTGAATCCTCAGGTATCTGGTCAGCAGGGGGG
GGTCCAGGCTGTTCACCACAGGGGTGAAGCTGTCCTGGTTGCCCTGGAACACCTTCACCTTGCCATTC
TGGAAGAACAGGGTCCACTGGTGGCCATCCTGGCTGCTGCTGATCAGGAACTCCTTCACATACATGCT
GGTCAGCAGGCTCTTCACCCCCTGGGTGGTCACCCCAGTCACCTTCATGGTCTTCTGGAAGTCCACCT
GCAGCCACTCCTTGGGGTTGTTGACCTGGGGCCTCCAGGCATTGCTCCTGCCCTGCAGGTGCAGCCTG
GCCTTGCTGGGGCTCCAGGTGGCAAACATGTTGGTGAAGTAGCTGCTGGCAGTGATCTGGGCATCAGA
GATGGCCTTGCTCTCCATGCCCAGGGGCATGCTGCAGCTGTTCAGGTCACAGCCCATCAGCTCCATCC
TCAGGGTGCTCCTGATGCTGTAGTGGGTGGGGTGCAGCCTGATGTATCTGGCAATGATGGGGGGGTTG
AAGATGTTGTGCTTGATGCCAGAGCTGTCCACATTGCCAAAGAACACCATCAGGGTGCCAGTGCTGTT
GCCCCTGTAGGTCTGCCACTTCTTGCCATCCAGGCTGTACATGATGATGAACTGGCTGATGTACAGGC
TGCTGAACTTCTGCCTGGCCCCCTGGGTCTTGATGCCATGGATGATCATGGGGGCCAGCAGGTCCACC
TTGATCCAGCTGAAGGGCTCCTTGGTGCTCCAGGCATTGATGCTGCCAGAGTAGTGCAGCCTGGCCAG
CTTGGGGGCCCACTGGCCATACTGGCCAGAGGCAGTGATCTGGAAGTCCCTGATGTGGCCAGAGGCCA
TGCCCAGGGGGGTCTGGCACTTGTTGCTGTACACCAGGAACAGGGTGCTCATGCCAGCATGCAGGTGC
TCCCCAATCAGGCACTCCACCCTCCAGATGCCAGCCTTGCTGGGCAGCATCTCCACAGTCTCAAACAC
CCCAGGGTACAGGTTGTACAGGGCCATCTTGTACTCCTCCTTCTTCCTCACAGTGAACACATGGCCAG
AGAAGTGGATGCTGTGGATGTTCTCATTGCTGCCCATGCTCAGCAGGTACCACCTGATCCTCTGGTCC
TGGGCCATCACCAGGCCAGGCAGGGTGTCCATGATGTAGCCATTGATGGCATGGAACCTGTAGTTCTC
CTTGAAGGTGGGGTCCTCCATCTGGATGTTGCAGGGGGCCCTGCAGTTCCTCTCCATGTTCTCAGTGA
AGTACCAGCTCTTGGTTTCATCAAAGATGGTGAAGAACAGGGCAAACTCCTGCACAGTCACCTGCCTG
CCATGGGCAGGGTTCAGGGTGTTGGTGTGGCACACCAGCAGGGGGCCAATCAGGCCAGAGTGCACATC
CTTCTCCAGGTCCACATCAGAGAAGTAGGCCCAGGCCTTGCAGTCAAACTCATCCTTGGTGGGGGCCA
TGTGGTGCTGCACCTTCCAGAAGTAGGTCTTGGTTTCATTGGGCTTCACAAAGTTCTTCCTGGGCTCA
GCCCCCTGCCTCTGGTCCTCCTCATAGCTGATCAGGCTGCTGTAGAAGCTGTAGGGCCTGCTGGCCTG
GTTCCTGAAGGTCACCATGATGTTGTCCTCCACCTCAGCCCTGATGTAGGGGCCCAGCAGGCCCAGGT
GCTCATTCAGCTCCCCTCTGTACAGGGGCTGGGTGAAGCTGCCATCAGTGAACTCCTGGAACACCACC
TTCTTGAACTGGGGCACAGAGCCAGACTGGGCCCTGTTCCTCAGCACATGGGGGCTGCTGCTCATGCC
ATAGTCCCACAGCCTCTCCACAGCAGCAATGAAGTAGTGCCTGGTCTTCTTCTGGAAGCTCCTGGGGC
TCTGGTTCTCGTCCTCGTCGTAGATGTCAAAGTCCTCCTTCTTCATCTCCACAGAGATGGTGTCATCA
TAGTCAATCTCCTCCTGGTCAGACTGCAGGGTGGTATTCTCTGGGATGGTGGTGGCATTGAACTGCTT
CTGCCTGGTGCTGGGGTGCCTGCTATTCTGGCTGAAGCTCCTGGGCTCAATGGCATTGTTCTTGCTCA
GCAGGTAGGCAGAGATGTCCTCATAGCTGTCCTCATAGTAGTCCCCAGTGTTCTTGTCACAGCTGGAG
ACTTTCAGCAGGGCAGTCATGCCCCTGTTCCTGAAGTCAGAGTTGTGGCAGCCCAGAATCCACAGGCC
AGGGTTCTCCATGCTCATGAACACAGTCTCCCCAGAGAAGGGGAACAGGGTCAGGGTGTCCTCATACA
CCATCTTGTGCTTGAAGGTGTAGCCAGAGAAGAACACAGACAGGAAGTCAGTCTGGGCCCCAATGCTC
AGGATGTACCAGTAGGCCACCTCATGCAGGCACACAGACAGCTGCAGGCTGTCAAACACATAGCCATT
GATGCTGTGCATGATGTTGCTGGCCTGGAACTCAGGGTCCTCCAGCTGCACCCCAGCAGGGTTGGGCA
GGAACCTCTGGATGTTCTCAGTCAGGTACCAGCTCCTGTTCTCATCAAACACAGAGAACAGGATCACA
TTCCTCTTGTCAGACATGATCTGGTTGCCCCTCTGGTCCACAGACTCCTTGTAGCAGATCAGCAGGGG
GCCAATCAGGCCAGAGGCCAGGTCCCTCTCCATGTTCACAAAGCTGCTGTAGTATCTGGTCAGGCACC
TGGGGTCAGACTTGGTGGGGCCATCCTCCACAGTCACAGTCCACTTGTACTTGAAGATCTCCCCAGGC
AGGATGGGGAAGTCCTTCAGGTGCTTCACCCCCTTGGGCAGCCTCCTGCTGTACAGGGGCCTCACATC
AGTGATGCCATGGGGGTAGATGTTGTAGGGCCTGCTGGCCTGGTTCTTGAAGATGATCAGCAGGGTGT
CCCCCACCTCCCCATACAGCAGGGGGCCCAGGATGCCAGACTCATGCTGGATGGCCTCCCTGGTCTTG
AAGGTTTCATCAGTGTAGGCCATGAACCTGACCTTCTTGTACTTCCTGCCAATCCTCTGGGGGCCATT
GTTCAGGTACTGGCTCTTGTAGCTCCTGTCATCAGGGGCCAGCACCAGGGGGGCATAGTCCCAGTCCT
CCTCCTCAGCAGCAATGTAGTGCACCCAGGTCTTGGGGTGCTTCTTGGCCACAGACCTGATCTGGATG
AAGCTGGGGCTGTTGTCATCATCAAACCTCACCACATCCATCTCAGAGTCAGTCAGGTCATCATCATA
GTCCTCAGCCTCCTCATTGTTCTTCATCCTCAGCTGGGGCTCCTCAGGGCAGCTGTCCACCTTCACAT
AGGCCTCCATGCCATCATGCTGGTGGCTGCTGATGTGGCAGAACAGCAGGAACTGGCCCAGGTCCATC
AGCAGGGTCTGGGCAGTCAGGAAGGTGATGGGGCTGATCTCCAGGCTGGCCTGCCTGTGGTTCCTGAC
CAGGAAGGTGTGGCCCTCCAGGAAGATGCTGTGCACCTCAGGGGTGGTGCCCATGCCAATCACATGCC
AGTACACAGACTTCCTGTGGCAGCCAATCAGGCCAGGCAGGCTCCTGTTCACATAGCCATTCACAGTG
TGCATCTTGGGCCAGGCCCTGGCAGAGGCAGCATCCCTGTCCTGCATCAGGCTGTTCTTGGTTTCAGA
GTGCCAGCTCTTGCCCTCATCAAACACAGCAAACAGCAGGATGAACTTGTGCAGGGTCTGGGTCTTCT
CCTTGGCCAGGCTGCCCTCCCTGCACACCAGCAGGGCCCCAATCAGGCCAGAGTTCAGGTCCTTCACC
AGGTCCACATGGCTCAGGTAGCTGTAGGTCAGGCACAGGGGGTCAGAGGCCATGGGGCCATTCTCCTT
CAGCACCTGCCACACATAGGTGTGGCTGCCCCCAGGGAACACCTTGTCATCCTCCTTCTCCCTCTGGC
TGGTCTGGTCATCATACTCAGCCCCCTCAGAGGCCTTCCAGTAGCTCACCCCCACAGCATGCAGGCTC
ACAGGGTGGCTGGCCATGTTCTTCAGGGTGATCACCACAGTGTCATACACCTCAGCCTGGATGGTGGG
GCCCAGCAGGCCCATCCAGGGGGGCCTGGGCTTGGCAATGTTGAACAGGTGGTCAGTGAACTCCACAA
ACAGGGTCTTCTTGTACACCACAGAGGTGTTGAAGGGGAAGCTCTTGGGCACTCTGGGGGGGAACCTG
GCATCCACAGGCAGCTCCCCCAGGTCAGACTGCATGTAGTCCCAGCTCAGCTCCACAGCCCCCAGGTA
GTATCTCCTGGTGGCAGAGAAGCAGAACCTCAGCAGGCACAGGAAGAAGCAGGTGCTCAGCTCAATCT
GCATGGTGGCGGCGTTTAAACCAACCTGAAAAAAAGTGATTTCAGGCAGGTGCTCCAGGTAATTAAAC
ATTAATACCCCACCAACCAACCATCCCTTAAACCCTTACCTCTTCAGGAGCTTGTGGATCTGTGTGAC
GGCTTCTCCTGGTGAAGGGGCTTTTATACCCCCTCCTTCCAACCCAGGCTGCTGATCCCTGCCAAGCT
GACTCCAAACCTGCTGATTCTGATTATTGACTTAGTCAACAAAAGGAGAATAAGTAACCTACACAAAT
ATGAACCTTGCCTAGGGAGATTAGAGTATCGGAACACTCGCTCTACGAAATGTGCAGACAGACGGGGA
TCCTCTAGAGCTACCTGCTGATCGCCCGGCCCCTGTTCAAACATGTCCTAATACTCTGTCTCTGCAAG
GGTCATCAGTAGTTTTCCATCTTACTCAACATCCTCCCAGTGGGTACCGTGGACTTAGCCCCTGTTTG
CTCCTCCGATAACTGGGGTGACCTTGGTTAATATTCACCAGCAGCCTCCCCCGGTGGACTTAGCCCCT
GTTTGCTCCTCCGATAACTGGGGTGACCTTGGTTAATATTCACCAGCAGCCTCCCCCGGTGGACTTAG
CCCCTGTTTGCTCCTCCGATAACTGGGGTGACCTTGGTTAATATTCACCAGCAGCCTCCCCCGCGGCC
GCGATAAGTAGCATGGCGGGTTAATCATTAACTACAAGGAACCCCTAGTGATGGAGCCTCAGCTTGGC
CACTCCCTCTCTGCGCGCTCGCTCGCTCACTGAGGCCGCCCGGGCAAAGCCCGGGCGTCGGGCGACCT
TTGGTCGCCCGIC

The nucleic acid sequence of oligo-037 and oligo-267 are shown below as SEQ ID NO: 4 and SEQ ID NO: 5, respectively. Oligo-267 has the same sequence as oligo-037, except for an inosine modification at −7 position.

Oligo-037 (61 bp; left ITR)
SEQ ID NO: 4
CTCACTGAGGCCGCCCGGGCAAAGCCCGGGCGTCGGGCGACCTTTGGTCGCCCGGCCTCAG
Oligo-267 (61 bp)
SEQ ID NO: 5
CTCACTGAGGCCGCCCGGGCAAAGCCCGGGCGTCGGGCGACCTTTGGTCGCCCGICCTCAG
Oligo-039 (57 bp; left ITR)
SEQ ID NO: 6
CACTGAGGCCGCCCGGGCAAAGCCCGGGCGTCGGGCGACCTTTGGTCGCCCGGCCTC
Oligo-002 (61 bp; left ITR)
SEQ ID NO: 7
CTCACTGAGGCCGCCCGGGCAAAGCCCGGGCGTCGGGCGACCTTTGGTCGCCCGGCCTCAG
Double stranded vector ds655 (6214 bp) is shown below as SEQ ID NO: 8
CTCACTGAGGCCGCCCGGGCAAAGCCCGGGCGTCGGGCGACCTTTGGTCGCCCGGCCTCAGTGAGCGA
GCGAGCGCGCAGAGAGGGAGTGGCCAAGCTGAGGCTCCATCACTAGGGGTTCCTTGTAGTTAATGATT
AACCCGCCATGCTACTTATCGCGGCCGCGGGGGAGGCTGCTGGTGAATATTAACCAAGGTCACCCCAG
TTATCGGAGGAGCAAACAGGGGCTAAGTCCACCGGGGGAGGCTGCTGGTGAATATTAACCAAGGTCAC
CCCAGTTATCGGAGGAGCAAACAGGGGCTAAGTCCACCGGGGGAGGCTGCTGGTGAATATTAACCAAG
GTCACCCCAGTTATCGGAGGAGCAAACAGGGGCTAAGTCCACGGTACCCACTGGGAGGATGTTGAGTA
AGATGGAAAACTACTGATGACCCTTGCAGAGACAGAGTATTAGGACATGTTTGAACAGGGGCCGGGCG
ATCAGCAGGTAGCTCTAGAGGATCCCCGTCTGTCTGCACATTTCGTAGAGCGAGTGTTCCGATACTCT
AATCTCCCTAGGCAAGGTTCATATTTGTGTAGGTTACTTATTCTCCTTTTGTTGACTAAGTCAATAAT
CAGAATCAGCAGGTTTGGAGTCAGCTTGGCAGGGATCAGCAGCCTGGGTTGGAAGGAGGGGGTATAAA
AGCCCCTTCACCAGGAGAAGCCGTCACACAGATCCACAAGCTCCTGAAGAGGTAAGGGTTTAAGGGAT
GGTTGGTTGGTGGGGTATTAATGTTTAATTACCTGGAGCACCTGCCTGAAATCACTTTTTTTCAGGTT
GGTTTAAACGCCGCCACCATGCAGATTGAGCTGAGCACCTGCTTCTTCCTGTGCCTGCTGAGGTTCTG
CTTCTCTGCCACCAGGAGATACTACCTGGGGGCTGTGGAGCTGAGCTGGGACTACATGCAGTCTGACC
TGGGGGAGCTGCCTGTGGATGCCAGGTTCCCCCCCAGAGTGCCCAAGAGCTTCCCCTTCAACACCTCT
GTGGTGTACAAGAAGACCCTGTTTGTGGAGTTCACTGACCACCTGTTCAACATTGCCAAGCCCAGGCC
CCCCTGGATGGGCCTGCTGGGCCCCACCATCCAGGCTGAGGTGTATGACACTGTGGTGATCACCCTGA
AGAACATGGCCAGCCACCCTGTGAGCCTGCATGCTGTGGGGGTGAGCTACTGGAAGGCCTCTGAGGGG
GCTGAGTATGATGACCAGACCAGCCAGAGGGAGAAGGAGGATGACAAGGTGTTCCCTGGGGGCAGCCA
CACCTATGTGTGGCAGGTGCTGAAGGAGAATGGCCCCATGGCCTCTGACCCCCTGTGCCTGACCTACA
GCTACCTGAGCCATGTGGACCTGGTGAAGGACCTGAACTCTGGCCTGATTGGGGCCCTGCTGGTGTGC
AGGGAGGGCAGCCTGGCCAAGGAGAAGACCCAGACCCTGCACAAGTTCATCCTGCTGTTTGCTGTGTT
TGATGAGGGCAAGAGCTGGCACTCTGAAACCAAGAACAGCCTGATGCAGGACAGGGATGCTGCCTCTG
CCAGGGCCTGGCCCAAGATGCACACTGTGAATGGCTATGTGAACAGGAGCCTGCCTGGCCTGATTGGC
TGCCACAGGAAGTCTGTGTACTGGCATGTGATTGGCATGGGCACCACCCCTGAGGTGCACAGCATCTT
CCTGGAGGGCCACACCTTCCTGGTCAGGAACCACAGGCAGGCCAGCCTGGAGATCAGCCCCATCACCT
TCCTGACTGCCCAGACCCTGCTGATGGACCTGGGCCAGTTCCTGCTGTTCTGCCACATCAGCAGCCAC
CAGCATGATGGCATGGAGGCCTATGTGAAGGTGGACAGCTGCCCTGAGGAGCCCCAGCTGAGGATGAA
GAACAATGAGGAGGCTGAGGACTATGATGATGACCTGACTGACTCTGAGATGGATGTGGTGAGGTTTG
ATGATGACAACAGCCCCAGCTTCATCCAGATCAGGTCTGTGGCCAAGAAGCACCCCAAGACCTGGGTG
CACTACATTGCTGCTGAGGAGGAGGACTGGGACTATGCCCCCCTGGTGCTGGCCCCTGATGACAGGAG
CTACAAGAGCCAGTACCTGAACAATGGCCCCCAGAGGATTGGCAGGAAGTACAAGAAGGTCAGGTTCA
TGGCCTACACTGATGAAACCTTCAAGACCAGGGAGGCCATCCAGCATGAGTCTGGCATCCTGGGCCCC
CTGCTGTATGGGGAGGTGGGGGACACCCTGCTGATCATCTTCAAGAACCAGGCCAGCAGGCCCTACAA
CATCTACCCCCATGGCATCACTGATGTGAGGCCCCTGTACAGCAGGAGGCTGCCCAAGGGGGTGAAGC
ACCTGAAGGACTTCCCCATCCTGCCTGGGGAGATCTTCAAGTACAAGTGGACTGTGACTGTGGAGGAT
GGCCCCACCAAGTCTGACCCCAGGTGCCTGACCAGATACTACAGCAGCTTTGTGAACATGGAGAGGGA
CCTGGCCTCTGGCCTGATTGGCCCCCTGCTGATCTGCTACAAGGAGTCTGTGGACCAGAGGGGCAACC
AGATCATGTCTGACAAGAGGAATGTGATCCTGTTCTCTGTGTTTGATGAGAACAGGAGCTGGTACCTG
ACTGAGAACATCCAGAGGTTCCTGCCCAACCCTGCTGGGGTGCAGCTGGAGGACCCTGAGTTCCAGGC
CAGCAACATCATGCACAGCATCAATGGCTATGTGTTTGACAGCCTGCAGCTGTCTGTGTGCCTGCATG
AGGTGGCCTACTGGTACATCCTGAGCATTGGGGCCCAGACTGACTTCCTGTCTGTGTTCTTCTCTGGC
TACACCTTCAAGCACAAGATGGTGTATGAGGACACCCTGACCCTGTTCCCCTTCTCTGGGGAGACTGT
GTTCATGAGCATGGAGAACCCTGGCCTGTGGATTCTGGGCTGCCACAACTCTGACTTCAGGAACAGGG
GCATGACTGCCCTGCTGAAAGTCTCCAGCTGTGACAAGAACACTGGGGACTACTATGAGGACAGCTAT
GAGGACATCTCTGCCTACCTGCTGAGCAAGAACAATGCCATTGAGCCCAGGAGCTTCAGCCAGAATAG
CAGGCACCCCAGCACCAGGCAGAAGCAGTTCAATGCCACCACCATCCCAGAGAATACCACCCTGCAGT
CTGACCAGGAGGAGATTGACTATGATGACACCATCTCTGTGGAGATGAAGAAGGAGGACTTTGACATC
TACGACGAGGACGAGAACCAGAGCCCCAGGAGCTTCCAGAAGAAGACCAGGCACTACTTCATTGCTGC
TGTGGAGAGGCTGTGGGACTATGGCATGAGCAGCAGCCCCCATGTGCTGAGGAACAGGGCCCAGTCTG
GCTCTGTGCCCCAGTTCAAGAAGGTGGTGTTCCAGGAGTTCACTGATGGCAGCTTCACCCAGCCCCTG
TACAGAGGGGAGCTGAATGAGCACCTGGGCCTGCTGGGCCCCTACATCAGGGCTGAGGTGGAGGACAA
CATCATGGTGACCTTCAGGAACCAGGCCAGCAGGCCCTACAGCTTCTACAGCAGCCTGATCAGCTATG
AGGAGGACCAGAGGCAGGGGGCTGAGCCCAGGAAGAACTTTGTGAAGCCCAATGAAACCAAGACCTAC
TTCTGGAAGGTGCAGCACCACATGGCCCCCACCAAGGATGAGTTTGACTGCAAGGCCTGGGCCTACTT
CTCTGATGTGGACCTGGAGAAGGATGTGCACTCTGGCCTGATTGGCCCCCTGCTGGTGTGCCACACCA
ACACCCTGAACCCTGCCCATGGCAGGCAGGTGACTGTGCAGGAGTTTGCCCTGTTCTTCACCATCTTT
GATGAAACCAAGAGCTGGTACTTCACTGAGAACATGGAGAGGAACTGCAGGGCCCCCTGCAACATCCA
GATGGAGGACCCCACCTTCAAGGAGAACTACAGGTTCCATGCCATCAATGGCTACATCATGGACACCC
TGCCTGGCCTGGTGATGGCCCAGGACCAGAGGATCAGGTGGTACCTGCTGAGCATGGGCAGCAATGAG
AACATCCACAGCATCCACTTCTCTGGCCATGTGTTCACTGTGAGGAAGAAGGAGGAGTACAAGATGGC
CCTGTACAACCTGTACCCTGGGGTGTTTGAGACTGTGGAGATGCTGCCCAGCAAGGCTGGCATCTGGA
GGGTGGAGTGCCTGATTGGGGAGCACCTGCATGCTGGCATGAGCACCCTGTTCCTGGTGTACAGCAAC
AAGTGCCAGACCCCCCTGGGCATGGCCTCTGGCCACATCAGGGACTTCCAGATCACTGCCTCTGGCCA
GTATGGCCAGTGGGCCCCCAAGCTGGCCAGGCTGCACTACTCTGGCAGCATCAATGCCTGGAGCACCA
AGGAGCCCTTCAGCTGGATCAAGGTGGACCTGCTGGCCCCCATGATCATCCATGGCATCAAGACCCAG
GGGGCCAGGCAGAAGTTCAGCAGCCTGTACATCAGCCAGTTCATCATCATGTACAGCCTGGATGGCAA
GAAGTGGCAGACCTACAGGGGCAACAGCACTGGCACCCTGATGGTGTTCTTTGGCAATGTGGACAGCT
CTGGCATCAAGCACAACATCTTCAACCCCCCCATCATTGCCAGATACATCAGGCTGCACCCCACCCAC
TACAGCATCAGGAGCACCCTGAGGATGGAGCTGATGGGCTGTGACCTGAACAGCTGCAGCATGCCCCT
GGGCATGGAGAGCAAGGCCATCTCTGATGCCCAGATCACTGCCAGCAGCTACTTCACCAACATGTTTG
CCACCTGGAGCCCCAGCAAGGCCAGGCTGCACCTGCAGGGCAGGAGCAATGCCTGGAGGCCCCAGGTC
AACAACCCCAAGGAGTGGCTGCAGGTGGACTTCCAGAAGACCATGAAGGTGACTGGGGTGACCACCCA
GGGGGTGAAGAGCCTGCTGACCAGCATGTATGTGAAGGAGTTCCTGATCAGCAGCAGCCAGGATGGCC
ACCAGTGGACCCTGTTCTTCCAGAATGGCAAGGTGAAGGTGTTCCAGGGCAACCAGGACAGCTTCACC
CCTGTGGTGAACAGCCTGGACCCCCCCCTGCTGACCAGATACCTGAGGATTCACCCCCAGAGCTGGGT
GCACCAGATTGCCCTGAGGATGGAGGTGCTGGGCTGTGAGGCCCAGGACCTGTACTGATTAATTAAGA
GCATCTTACCGCCATTTATTCCCATATTTGTTCTGTTTTTCTTGATTTGGGTATACATTTAAATGTTA
ATAAAACAAAATGGTGGGGCAATCATTTACATTTTTAGGGATATGTAATTACTAGTTCAGGTGTATTG
CCACAAGACAAACATGTTAAGAAACTTTCCCGTTATTTACGCTCTGTTCCTGTTAATCAACCTCTGGA
TTACAAAATTTGTGAAAGATTGACTGATATTCTTAACTATGTTGCTCCTTTTACGCTGTGTGGATATG
CTGCTTTATAGCCTCTGTATCTAGCTATTGCTTCCCGTACGGCTTTCGTTTTCTCCTCCTTGTATAAA
TCCTGGTTGCTGTCTCTTTTAGAGGAGTTGTGGCCCGTTGTCCGTCAACGTGGCGTGGTGTGCTCTGT
GTTTGCTGACGCAACCCCCACTGGCTGGGGCATTGCCACCACCTGTCAACTCCTTTCTGGGACTTTCG
CTTTCCCCCTCCCGATCGCCACGGCAGAACTCATCGCCGCCTGCCTTGCCCGCTGCTGGACAGGGGCT
AGGTTGCTGGGCACTGATAATTCCGTGGTGTTGTCTGTGCCTTCTAGTTGCCAGCCATCTGTTGTTTG
CCCCTCCCCCGTGCCTTCCTTGACCCTGGAAGGTGCCACTCCCACTGTCCTTTCCTAATAAAATGAGG
AAATTGCATCGCATTGTCTGAGTAGGTGTCATTCTATTCTGGGGGGTGGGGTGGGGCAGGACAGCAAG
GGGGAGGATTGGGAAGACAATAGCAGGCATGCTGGGGATGCGGTGGGCTCTATGGCTCTAGAGCATGG
CTACGTAGATAAGTAGCATGGCGGGTTAATCATTAACTACACCTGCAGGAGGAACCCCTAGTGATGGA
GTTGGCCACTCCCTCTCTGCGCGCTCGCTCGCTCACTGAGGCCGGGCGACCAAAGGTCGCCCGACGCC
CGGGCTTTGCCCGGGCGGCCTCAGTG
Double stranded vector ds656 (6214 bp) is shown below as SEQ ID NO: 9
CTCACTGAGGCCGCCCGGGCAAAGCCCGGGCGTCGGGCGACCTTTGGTCGCCCGNCCTCAGTGAGCGA
GCGAGCGCGCAGAGAGGGAGTGGCCAAGCTGAGGCTCCATCACTAGGGGTTCCTTGTAGTTAATGATT
AACCCGCCATGCTACTTATCGCGGCCGCGGGGGAGGCTGCTGGTGAATATTAACCAAGGTCACCCCAG
TTATCGGAGGAGCAAACAGGGGCTAAGTCCACCGGGGGAGGCTGCTGGTGAATATTAACCAAGGTCAC
CCCAGTTATCGGAGGAGCAAACAGGGGCTAAGTCCACCGGGGGAGGCTGCTGGTGAATATTAACCAAG
GTCACCCCAGTTATCGGAGGAGCAAACAGGGGCTAAGTCCACGGTACCCACTGGGAGGATGTTGAGTA
AGATGGAAAACTACTGATGACCCTTGCAGAGACAGAGTATTAGGACATGTTTGAACAGGGGCCGGGCG
ATCAGCAGGTAGCTCTAGAGGATCCCCGTCTGTCTGCACATTTCGTAGAGCGAGTGTTCCGATACTCT
AATCTCCCTAGGCAAGGTTCATATTTGTGTAGGTTACTTATTCTCCTTTTGTTGACTAAGTCAATAAT
CAGAATCAGCAGGTTTGGAGTCAGCTTGGCAGGGATCAGCAGCCTGGGTTGGAAGGAGGGGGTATAAA
AGCCCCTTCACCAGGAGAAGCCGTCACACAGATCCACAAGCTCCTGAAGAGGTAAGGGTTTAAGGGAT
GGTTGGTTGGTGGGGTATTAATGTTTAATTACCTGGAGCACCTGCCTGAAATCACTTTTTTTCAGGTT
GGTTTAAACGCCGCCACCATGCAGATTGAGCTGAGCACCTGCTTCTTCCTGTGCCTGCTGAGGTTCTG
CTTCTCTGCCACCAGGAGATACTACCTGGGGGCTGTGGAGCTGAGCTGGGACTACATGCAGTCTGACC
TGGGGGAGCTGCCTGTGGATGCCAGGTTCCCCCCCAGAGTGCCCAAGAGCTTCCCCTTCAACACCTCT
GTGGTGTACAAGAAGACCCTGTTTGTGGAGTTCACTGACCACCTGTTCAACATTGCCAAGCCCAGGCC
CCCCTGGATGGGCCTGCTGGGCCCCACCATCCAGGCTGAGGTGTATGACACTGTGGTGATCACCCTGA
AGAACATGGCCAGCCACCCTGTGAGCCTGCATGCTGTGGGGGTGAGCTACTGGAAGGCCTCTGAGGGG
GCTGAGTATGATGACCAGACCAGCCAGAGGGAGAAGGAGGATGACAAGGTGTTCCCTGGGGGCAGCCA
CACCTATGTGTGGCAGGTGCTGAAGGAGAATGGCCCCATGGCCTCTGACCCCCTGTGCCTGACCTACA
GCTACCTGAGCCATGTGGACCTGGTGAAGGACCTGAACTCTGGCCTGATTGGGGCCCTGCTGGTGTGC
AGGGAGGGCAGCCTGGCCAAGGAGAAGACCCAGACCCTGCACAAGTTCATCCTGCTGTTTGCTGTGTT
TGATGAGGGCAAGAGCTGGCACTCTGAAACCAAGAACAGCCTGATGCAGGACAGGGATGCTGCCTCTG
CCAGGGCCTGGCCCAAGATGCACACTGTGAATGGCTATGTGAACAGGAGCCTGCCTGGCCTGATTGGC
TGCCACAGGAAGTCTGTGTACTGGCATGTGATTGGCATGGGCACCACCCCTGAGGTGCACAGCATCTT
CCTGGAGGGCCACACCTTCCTGGTCAGGAACCACAGGCAGGCCAGCCTGGAGATCAGCCCCATCACCT
TCCTGACTGCCCAGACCCTGCTGATGGACCTGGGCCAGTTCCTGCTGTTCTGCCACATCAGCAGCCAC
CAGCATGATGGCATGGAGGCCTATGTGAAGGTGGACAGCTGCCCTGAGGAGCCCCAGCTGAGGATGAA
GAACAATGAGGAGGCTGAGGACTATGATGATGACCTGACTGACTCTGAGATGGATGTGGTGAGGTTTG
ATGATGACAACAGCCCCAGCTTCATCCAGATCAGGTCTGTGGCCAAGAAGCACCCCAAGACCTGGGTG
CACTACATTGCTGCTGAGGAGGAGGACTGGGACTATGCCCCCCTGGTGCTGGCCCCTGATGACAGGAG
CTACAAGAGCCAGTACCTGAACAATGGCCCCCAGAGGATTGGCAGGAAGTACAAGAAGGTCAGGTTCA
TGGCCTACACTGATGAAACCTTCAAGACCAGGGAGGCCATCCAGCATGAGTCTGGCATCCTGGGCCCC
CTGCTGTATGGGGAGGTGGGGGACACCCTGCTGATCATCTTCAAGAACCAGGCCAGCAGGCCCTACAA
CATCTACCCCCATGGCATCACTGATGTGAGGCCCCTGTACAGCAGGAGGCTGCCCAAGGGGGTGAAGC
ACCTGAAGGACTTCCCCATCCTGCCTGGGGAGATCTTCAAGTACAAGTGGACTGTGACTGTGGAGGAT
GGCCCCACCAAGTCTGACCCCAGGTGCCTGACCAGATACTACAGCAGCTTTGTGAACATGGAGAGGGA
CCTGGCCTCTGGCCTGATTGGCCCCCTGCTGATCTGCTACAAGGAGTCTGTGGACCAGAGGGGCAACC
AGATCATGTCTGACAAGAGGAATGTGATCCTGTTCTCTGTGTTTGATGAGAACAGGAGCTGGTACCTG
ACTGAGAACATCCAGAGGTTCCTGCCCAACCCTGCTGGGGTGCAGCTGGAGGACCCTGAGTTCCAGGC
CAGCAACATCATGCACAGCATCAATGGCTATGTGTTTGACAGCCTGCAGCTGTCTGTGTGCCTGCATG
AGGTGGCCTACTGGTACATCCTGAGCATTGGGGCCCAGACTGACTTCCTGTCTGTGTTCTTCTCTGGC
TACACCTTCAAGCACAAGATGGTGTATGAGGACACCCTGACCCTGTTCCCCTTCTCTGGGGAGACTGT
GTTCATGAGCATGGAGAACCCTGGCCTGTGGATTCTGGGCTGCCACAACTCTGACTTCAGGAACAGGG
GCATGACTGCCCTGCTGAAAGTCTCCAGCTGTGACAAGAACACTGGGGACTACTATGAGGACAGCTAT
GAGGACATCTCTGCCTACCTGCTGAGCAAGAACAATGCCATTGAGCCCAGGAGCTTCAGCCAGAATAG
CAGGCACCCCAGCACCAGGCAGAAGCAGTTCAATGCCACCACCATCCCAGAGAATACCACCCTGCAGT
CTGACCAGGAGGAGATTGACTATGATGACACCATCTCTGTGGAGATGAAGAAGGAGGACTTTGACATC
TACGACGAGGACGAGAACCAGAGCCCCAGGAGCTTCCAGAAGAAGACCAGGCACTACTTCATTGCTGC
TGTGGAGAGGCTGTGGGACTATGGCATGAGCAGCAGCCCCCATGTGCTGAGGAACAGGGCCCAGTCTG
GCTCTGTGCCCCAGTTCAAGAAGGTGGTGTTCCAGGAGTTCACTGATGGCAGCTTCACCCAGCCCCTG
TACAGAGGGGAGCTGAATGAGCACCTGGGCCTGCTGGGCCCCTACATCAGGGCTGAGGTGGAGGACAA
CATCATGGTGACCTTCAGGAACCAGGCCAGCAGGCCCTACAGCTTCTACAGCAGCCTGATCAGCTATG
AGGAGGACCAGAGGCAGGGGGCTGAGCCCAGGAAGAACTTTGTGAAGCCCAATGAAACCAAGACCTAC
TTCTGGAAGGTGCAGCACCACATGGCCCCCACCAAGGATGAGTTTGACTGCAAGGCCTGGGCCTACTT
CTCTGATGTGGACCTGGAGAAGGATGTGCACTCTGGCCTGATTGGCCCCCTGCTGGTGTGCCACACCA
ACACCCTGAACCCTGCCCATGGCAGGCAGGTGACTGTGCAGGAGTTTGCCCTGTTCTTCACCATCTTT
GATGAAACCAAGAGCTGGTACTTCACTGAGAACATGGAGAGGAACTGCAGGGCCCCCTGCAACATCCA
GATGGAGGACCCCACCTTCAAGGAGAACTACAGGTTCCATGCCATCAATGGCTACATCATGGACACCC
TGCCTGGCCTGGTGATGGCCCAGGACCAGAGGATCAGGTGGTACCTGCTGAGCATGGGCAGCAATGAG
AACATCCACAGCATCCACTTCTCTGGCCATGTGTTCACTGTGAGGAAGAAGGAGGAGTACAAGATGGC
CCTGTACAACCTGTACCCTGGGGTGTTTGAGACTGTGGAGATGCTGCCCAGCAAGGCTGGCATCTGGA
GGGTGGAGTGCCTGATTGGGGAGCACCTGCATGCTGGCATGAGCACCCTGTTCCTGGTGTACAGCAAC
AAGTGCCAGACCCCCCTGGGCATGGCCTCTGGCCACATCAGGGACTTCCAGATCACTGCCTCTGGCCA
GTATGGCCAGTGGGCCCCCAAGCTGGCCAGGCTGCACTACTCTGGCAGCATCAATGCCTGGAGCACCA
AGGAGCCCTTCAGCTGGATCAAGGTGGACCTGCTGGCCCCCATGATCATCCATGGCATCAAGACCCAG
GGGGCCAGGCAGAAGTTCAGCAGCCTGTACATCAGCCAGTTCATCATCATGTACAGCCTGGATGGCAA
GAAGTGGCAGACCTACAGGGGCAACAGCACTGGCACCCTGATGGTGTTCTTTGGCAATGTGGACAGCT
CTGGCATCAAGCACAACATCTTCAACCCCCCCATCATTGCCAGATACATCAGGCTGCACCCCACCCAC
TACAGCATCAGGAGCACCCTGAGGATGGAGCTGATGGGCTGTGACCTGAACAGCTGCAGCATGCCCCT
GGGCATGGAGAGCAAGGCCATCTCTGATGCCCAGATCACTGCCAGCAGCTACTTCACCAACATGTTTG
CCACCTGGAGCCCCAGCAAGGCCAGGCTGCACCTGCAGGGCAGGAGCAATGCCTGGAGGCCCCAGGTC
AACAACCCCAAGGAGTGGCTGCAGGTGGACTTCCAGAAGACCATGAAGGTGACTGGGGTGACCACCCA
GGGGGTGAAGAGCCTGCTGACCAGCATGTATGTGAAGGAGTTCCTGATCAGCAGCAGCCAGGATGGCC
ACCAGTGGACCCTGTTCTTCCAGAATGGCAAGGTGAAGGTGTTCCAGGGCAACCAGGACAGCTTCACC
CCTGTGGTGAACAGCCTGGACCCCCCCCTGCTGACCAGATACCTGAGGATTCACCCCCAGAGCTGGGT
GCACCAGATTGCCCTGAGGATGGAGGTGCTGGGCTGTGAGGCCCAGGACCTGTACTGATTAATTAAGA
GCATCTTACCGCCATTTATTCCCATATTTGTTCTGTTTTTCTTGATTTGGGTATACATTTAAATGTTA
ATAAAACAAAATGGTGGGGCAATCATTTACATTTTTAGGGATATGTAATTACTAGTTCAGGTGTATTG
CCACAAGACAAACATGTTAAGAAACTTTCCCGTTATTTACGCTCTGTTCCTGTTAATCAACCTCTGGA
TTACAAAATTTGTGAAAGATTGACTGATATTCTTAACTATGTTGCTCCTTTTACGCTGTGTGGATATG
CTGCTTTATAGCCTCTGTATCTAGCTATTGCTTCCCGTACGGCTTTCGTTTTCTCCTCCTTGTATAAA
TCCTGGTTGCTGTCTCTTTTAGAGGAGTTGTGGCCCGTTGTCCGTCAACGTGGCGTGGTGTGCTCTGT
GTTTGCTGACGCAACCCCCACTGGCTGGGGCATTGCCACCACCTGTCAACTCCTTTCTGGGACTTTCG
CTTTCCCCCTCCCGATCGCCACGGCAGAACTCATCGCCGCCTGCCTTGCCCGCTGCTGGACAGGGGCT
AGGTTGCTGGGCACTGATAATTCCGTGGTGTTGTCTGTGCCTTCTAGTTGCCAGCCATCTGTTGTTTG
CCCCTCCCCCGTGCCTTCCTTGACCCTGGAAGGTGCCACTCCCACTGTCCTTTCCTAATAAAATGAGG
AAATTGCATCGCATTGTCTGAGTAGGTGTCATTCTATTCTGGGGGGTGGGGTGGGGCAGGACAGCAAG
GGGGAGGATTGGGAAGACAATAGCAGGCATGCTGGGGATGCGGTGGGCTCTATGGCTCTAGAGCATGG
CTACGTAGATAAGTAGCATGGCGGGTTAATCATTAACTACACCTGCAGGAGGAACCCCTAGTGATGGA
GTTGGCCACTCCCTCTCTGCGCGCTCGCTCGCTCACTGAGGCCGGGCGACCAAAGGTCGCCCGACGCC
CGGGCTTTGCCCGGGCGGCCTCAGTG
Plasmid 704 (Sf9 Construct) (6040 bp) is shown below as SEQ ID NO: 10
CCTGCAGGCAGCTGCGCGCTCGCTCGCTCACTGAGGCCGCCCGGGCAAAGCCCGGGCGTCGGGCGACC
TTTGGTCGCCCGGCCTCAGTGAGCGAGCGAGCGCGCAGAGAGGGAGTGGCCAACTCCATCACTAGGGG
TTCCTTGTAGTTAATGATTAACCCGCCATGCTACTTATCTACGTAGCCATGCTCTAGACGGGGGAGGC
TGCTGGTGAATATTAACCAAGGTCACCCCAGTTATCGGAGGAGCAAACAGGGGCTAAGTCCACACGCG
TGGTACCGTCTGTCTGCACATTTCGTAGAGCGAGTGTTCCGATACTCTAATCTCCCTAGGCAAGGTTC
ATATTTGTGTAGGTTACTTATTCTCCTTTTGTTGACTAAGTCAATAATCAGAATCAGCAGGTTTGGAG
TCAGCTTGGCAGGGATCAGCAGCCTGGGTTGGAAGGAGGGGGTATAAAAGCCCCTTCACCAGGAGAAG
CCGTCACACAGATCCACAAGCTCCTGAAGAGGTAAGGGTTTAAGGGATGGTTGGTTGGTGGGGTATTA
ATGTTTAATTACCTGGAGCACCTGCCTGAAATCACTTTTTTTCAGGTTGGTTTAAACGCCGCCACCAT
GCAGATTGAGCTGAGCACCTGCTTCTTCCTGTGCCTGCTGAGGTTCTGCTTCTCTGCCACCAGGAGAT
ACTACCTGGGGGCTGTGGAGCTGAGCTGGGACTACATGCAGTCTGACCTGGGGGAGCTGCCTGTGGAT
GCCAGGTTCCCCCCCAGAGTGCCCAAGAGCTTCCCCTTCAACACCTCTGTGGTGTACAAGAAGACCCT
GTTTGTGGAGTTCACTGACCACCTGTTCAACATTGCCAAGCCCAGGCCCCCCTGGATGGGCCTGCTGG
GCCCCACCATCCAGGCTGAGGTGTATGACACTGTGGTGATCACCCTGAAGAACATGGCCAGCCACCCT
GTGAGCCTGCATGCTGTGGGGGTGAGCTACTGGAAGGCCTCTGAGGGGGCTGAGTATGATGACCAGAC
CAGCCAGAGGGAGAAGGAGGATGACAAGGTGTTCCCTGGGGGCAGCCACACCTATGTGTGGCAGGTGC
TGAAGGAGAATGGCCCCATGGCCTCTGACCCCCTGTGCCTGACCTACAGCTACCTGAGCCATGTGGAC
CTGGTGAAGGACCTGAACTCTGGCCTGATTGGGGCCCTGCTGGTGTGCAGGGAGGGCAGCCTGGCCAA
GGAGAAGACCCAGACCCTGCACAAGTTCATCCTGCTGTTTGCTGTGTTTGATGAGGGCAAGAGCTGGC
ACTCTGAAACCAAGAACAGCCTGATGCAGGACAGGGATGCTGCCTCTGCCAGGGCCTGGCCCAAGATG
CACACTGTGAATGGCTATGTGAACAGGAGCCTGCCTGGCCTGATTGGCTGCCACAGGAAGTCTGTGTA
CTGGCATGTGATTGGCATGGGCACCACCCCTGAGGTGCACAGCATCTTCCTGGAGGGCCACACCTTCC
TGGTCAGGAACCACAGGCAGGCCAGCCTGGAGATCAGCCCCATCACCTTCCTGACTGCCCAGACCCTG
CTGATGGACCTGGGCCAGTTCCTGCTGTTCTGCCACATCAGCAGCCACCAGCATGATGGCATGGAGGC
CTATGTGAAGGTGGACAGCTGCCCTGAGGAGCCCCAGCTGAGGATGAAGAACAATGAGGAGGCTGAGG
ACTATGATGATGACCTGACTGACTCTGAGATGGATGTGGTGAGGTTTGATGATGACAACAGCCCCAGC
TTCATCCAGATCAGGTCTGTGGCCAAGAAGCACCCCAAGACCTGGGTGCACTACATTGCTGCTGAGGA
GGAGGACTGGGACTATGCCCCCCTGGTGCTGGCCCCTGATGACAGGAGCTACAAGAGCCAGTACCTGA
ACAATGGCCCCCAGAGGATTGGCAGGAAGTACAAGAAGGTCAGGTTCATGGCCTACACTGATGAAACC
TTCAAGACCAGGGAGGCCATCCAGCATGAGTCTGGCATCCTGGGCCCCCTGCTGTATGGGGAGGTGGG
GGACACCCTGCTGATCATCTTCAAGAACCAGGCCAGCAGGCCCTACAACATCTACCCCCATGGCATCA
CTGATGTGAGGCCCCTGTACAGCAGGAGGCTGCCCAAGGGGGTGAAGCACCTGAAGGACTTCCCCATC
CTGCCTGGGGAGATCTTCAAGTACAAGTGGACTGTGACTGTGGAGGATGGCCCCACCAAGTCTGACCC
CAGGTGCCTGACCAGATACTACAGCAGCTTTGTGAACATGGAGAGGGACCTGGCCTCTGGCCTGATTG
GCCCCCTGCTGATCTGCTACAAGGAGTCTGTGGACCAGAGGGGCAACCAGATCATGTCTGACAAGAGG
AATGTGATCCTGTTCTCTGTGTTTGATGAGAACAGGAGCTGGTACCTGACTGAGAACATCCAGAGGTT
CCTGCCCAACCCTGCTGGGGTGCAGCTGGAGGACCCTGAGTTCCAGGCCAGCAACATCATGCACAGCA
TCAATGGCTATGTGTTTGACAGCCTGCAGCTGTCTGTGTGCCTGCATGAGGTGGCCTACTGGTACATC
CTGAGCATTGGGGCCCAGACTGACTTCCTGTCTGTGTTCTTCTCTGGCTACACCTTCAAGCACAAGAT
GGTGTATGAGGACACCCTGACCCTGTTCCCCTTCTCTGGGGAGACTGTGTTCATGAGCATGGAGAACC
CTGGCCTGTGGATTCTGGGCTGCCACAACTCTGACTTCAGGAACAGGGGCATGACTGCCCTGCTGAAA
GTCTCCAGCTGTGACAAGAACACTGGGGACTACTATGAGGACAGCTATGAGGACATCTCTGCCTACCT
GCTGAGCAAGAACAATGCCATTGAGCCCAGGAGCTTCAGCCAGAATGCCACTAATGTGTCTAACAACA
GCAACACCAGCAATGACAGCAATGTGTCTCCCCCAGTGCTGAAGAGGCACCAGAGGGAGATCACCAGG
ACCACCCTGCAGTCTGACCAGGAGGAGATTGACTATGATGACACCATCTCTGTGGAGATGAAGAAGGA
GGACTTTGACATCTACGACGAGGACGAGAACCAGAGCCCCAGGAGCTTCCAGAAGAAGACCAGGCACT
ACTTCATTGCTGCTGTGGAGAGGCTGTGGGACTATGGCATGAGCAGCAGCCCCCATGTGCTGAGGAAC
AGGGCCCAGTCTGGCTCTGTGCCCCAGTTCAAGAAGGTGGTGTTCCAGGAGTTCACTGATGGCAGCTT
CACCCAGCCCCTGTACAGAGGGGAGCTGAATGAGCACCTGGGCCTGCTGGGCCCCTACATCAGGGCTG
AGGTGGAGGACAACATCATGGTGACCTTCAGGAACCAGGCCAGCAGGCCCTACAGCTTCTACAGCAGC
CTGATCAGCTATGAGGAGGACCAGAGGCAGGGGGCTGAGCCCAGGAAGAACTTTGTGAAGCCCAATGA
AACCAAGACCTACTTCTGGAAGGTGCAGCACCACATGGCCCCCACCAAGGATGAGTTTGACTGCAAGG
CCTGGGCCTACTTCTCTGATGTGGACCTGGAGAAGGATGTGCACTCTGGCCTGATTGGCCCCCTGCTG
GTGTGCCACACCAACACCCTGAACCCTGCCCATGGCAGGCAGGTGACTGTGCAGGAGTTTGCCCTGTT
CTTCACCATCTTTGATGAAACCAAGAGCTGGTACTTCACTGAGAACATGGAGAGGAACTGCAGGGCCC
CCTGCAACATCCAGATGGAGGACCCCACCTTCAAGGAGAACTACAGGTTCCATGCCATCAATGGCTAC
ATCATGGACACCCTGCCTGGCCTGGTGATGGCCCAGGACCAGAGGATCAGGTGGTACCTGCTGAGCAT
GGGCAGCAATGAGAACATCCACAGCATCCACTTCTCTGGCCATGTGTTCACTGTGAGGAAGAAGGAGG
AGTACAAGATGGCCCTGTACAACCTGTACCCTGGGGTGTTTGAGACTGTGGAGATGCTGCCCAGCAAG
GCTGGCATCTGGAGGGTGGAGTGCCTGATTGGGGAGCACCTGCATGCTGGCATGAGCACCCTGTTCCT
GGTGTACAGCAACAAGTGCCAGACCCCCCTGGGCATGGCCTCTGGCCACATCAGGGACTTCCAGATCA
CTGCCTCTGGCCAGTATGGCCAGTGGGCCCCCAAGCTGGCCAGGCTGCACTACTCTGGCAGCATCAAT
GCCTGGAGCACCAAGGAGCCCTTCAGCTGGATCAAGGTGGACCTGCTGGCCCCCATGATCATCCATGG
CATCAAGACCCAGGGGGCCAGGCAGAAGTTCAGCAGCCTGTACATCAGCCAGTTCATCATCATGTACA
GCCTGGATGGCAAGAAGTGGCAGACCTACAGGGGCAACAGCACTGGCACCCTGATGGTGTTCTTTGGC
AATGTGGACAGCTCTGGCATCAAGCACAACATCTTCAACCCCCCCATCATTGCCAGATACATCAGGCT
GCACCCCACCCACTACAGCATCAGGAGCACCCTGAGGATGGAGCTGATGGGCTGTGACCTGAACAGCT
GCAGCATGCCCCTGGGCATGGAGAGCAAGGCCATCTCTGATGCCCAGATCACTGCCAGCAGCTACTTC
ACCAACATGTTTGCCACCTGGAGCCCCAGCAAGGCCAGGCTGCACCTGCAGGGCAGGAGCAATGCCTG
GAGGCCCCAGGTCAACAACCCCAAGGAGTGGCTGCAGGTGGACTTCCAGAAGACCATGAAGGTGACTG
GGGTGACCACCCAGGGGGTGAAGAGCCTGCTGACCAGCATGTATGTGAAGGAGTTCCTGATCAGCAGC
AGCCAGGATGGCCACCAGTGGACCCTGTTCTTCCAGAATGGCAAGGTGAAGGTGTTCCAGGGCAACCA
GGACAGCTTCACCCCTGTGGTGAACAGCCTGGACCCCCCCCTGCTGACCAGATACCTGAGGATTCACC
CCCAGAGCTGGGTGCACCAGATTGCCCTGAGGATGGAGGTGCTGGGCTGTGAGGCCCAGGACCTGTAC
TGATTAATTAAGAGCATCTTACCGCCATTTATTCCCATATTTGTTCTGTTTTTCTTGATTTGGGTATA
CATTTAAATGTTAATAAAACAAAATGGTGGGGCAATCATTTACATTTTTAGGGATATGTAATTACTAG
TTCAGGTGTATTGCCACAAGACAAACATGTTAAGAAACTTTCCCGTTATTTACGCTCTGTTCCTGTTA
ATCAACCTCTGGATTACAAAATTTGTGAAAGATTGACTGATATTCTTAACTATGTTGCTCCTTTTACG
CTGTGTGGATATGCTGCTTTATAGCCTCTGTATCTAGCTATTGCTTCCCGTACGGCTTTCGTTTTCTC
CTCCTTGTATAAATCCTGGTTGCTGTCTCTTTTAGAGGAGTTGTGGCCCGTTGTCCGTCAACGTGGCG
TGGTGTGCTCTGTGTTTGCTGACGCAACCCCCACTGGCTGGGGCATTGCCACCACCTGTCAACTCCTT
TCTGGGACTTTCGCTTTCCCCCTCCCGATCGCCACGGCAGAACTCATCGCCGCCTGCCTTGCCCGCTG
CTGGACAGGGGCTAGGTTGCTGGGCACTGATAATTCCGTGGTGTTGTCTGTGCCTTCTAGTTGCCAGC
CATCTGTTGTTTGCCCCTCCCCCGTGCCTTCCTTGACCCTGGAAGGTGCCACTCCCACTGTCCTTTCC
TAATAAAATGAGGAAATTGCATCGCATTGTCTGAGTAGGTGTCATTCTATTCTGGGGGGTGGGGTGGG
GCAGGACAGCAAGGGGGAGGATTGGGAAGACAATAGCAGGCATGCTGGGGATGCGGTGGGCTCTATGG
CTCTAGAGCATGGCTACGTAGATAAGTAGCATGGCGGGTTAATCATTAACTACACCTGCAGGAGGAAC
CCCTAGTGATGGAGTTGGCCACTCCCTCTCTGCGCGCTCGCTCGCTCACTGAGGCCGGGCGACCAAAG
GTCGCCCGACGCCCGGGCGGCCTCAGTGAGCGAGCGAGCGCGCAGCTGCCTGCAGG
Synthetic plasmid SP-543 (8199 bp) is shown below as SEQ ID NO: 11
AGGGGTTCCGCGCACATTTCCCCGAAAAGTGCCACCTGACGTCTTTCGTCAGATCACCGGGTTGTTCC
ATATCATCGTGTCCACAAGGGCTTGCCGGTCAAGTGCCTAAGCTCACTCGAGGGTCTCGTGAGCGAGC
GAGCGCGCAGAGAGGGAGTGGCCAAGCTGAGGCTCCATCACTAGGGGTTCCTTGTAGTTAATGATTAA
CCCGCCATGCTACTTATCGCGGCCGCGGGGGAGGCTGCTGGTGAATATTAACCAAGGTCACCCCAGTT
ATCGGAGGAGCAAACAGGGGCTAAGTCCACCGGGGGAGGCTGCTGGTGAATATTAACCAAGGTCACCC
CAGTTATCGGAGGAGCAAACAGGGGCTAAGTCCACCGGGGGAGGCTGCTGGTGAATATTAACCAAGGT
CACCCCAGTTATCGGAGGAGCAAACAGGGGCTAAGTCCACGGTACCCACTGGGAGGATGTTGAGTAAG
ATGGAAAACTACTGATGACCCTTGCAGAGACAGAGTATTAGGACATGTTTGAACAGGGGCCGGGCGAT
CAGCAGGTAGCTCTAGAGGATCCCCGTCTGTCTGCACATTTCGTAGAGCGAGTGTTCCGATACTCTAA
TCTCCCTAGGCAAGGTTCATATTTGTGTAGGTTACTTATTCTCCTTTTGTTGACTAAGTCAATAATCA
GAATCAGCAGGTTTGGAGTCAGCTTGGCAGGGATCAGCAGCCTGGGTTGGAAGGAGGGGGTATAAAAG
CCCCTTCACCAGGAGAAGCCGTCACACAGATCCACAAGCTCCTGAAGAGGTAAGGGTTTAAGGGATGG
TTGGTTGGTGGGGTATTAATGTTTAATTACCTGGAGCACCTGCCTGAAATCACTTTTTTTCAGGTTGG
TTTAAACGCCGCCACCATGCAGATTGAGCTGAGCACCTGCTTCTTCCTGTGCCTGCTGAGGTTCTGCT
TCTCTGCCACCAGGAGATACTACCTGGGGGCTGTGGAGCTGAGCTGGGACTACATGCAGTCTGACCTG
GGGGAGCTGCCTGTGGATGCCAGGTTCCCCCCCAGAGTGCCCAAGAGCTTCCCCTTCAACACCTCTGT
GGTGTACAAGAAGACCCTGTTTGTGGAGTTCACTGACCACCTGTTCAACATTGCCAAGCCCAGGCCCC
CCTGGATGGGCCTGCTGGGCCCCACCATCCAGGCTGAGGTGTATGACACTGTGGTGATCACCCTGAAG
AACATGGCCAGCCACCCTGTGAGCCTGCATGCTGTGGGGGTGAGCTACTGGAAGGCCTCTGAGGGGGC
TGAGTATGATGACCAGACCAGCCAGAGGGAGAAGGAGGATGACAAGGTGTTCCCTGGGGGCAGCCACA
CCTATGTGTGGCAGGTGCTGAAGGAGAATGGCCCCATGGCCTCTGACCCCCTGTGCCTGACCTACAGC
TACCTGAGCCATGTGGACCTGGTGAAGGACCTGAACTCTGGCCTGATTGGGGCCCTGCTGGTGTGCAG
GGAGGGCAGCCTGGCCAAGGAGAAGACCCAGACCCTGCACAAGTTCATCCTGCTGTTTGCTGTGTTTG
ATGAGGGCAAGAGCTGGCACTCTGAAACCAAGAACAGCCTGATGCAGGACAGGGATGCTGCCTCTGCC
AGGGCCTGGCCCAAGATGCACACTGTGAATGGCTATGTGAACAGGAGCCTGCCTGGCCTGATTGGCTG
CCACAGGAAGTCTGTGTACTGGCATGTGATTGGCATGGGCACCACCCCTGAGGTGCACAGCATCTTCC
TGGAGGGCCACACCTTCCTGGTCAGGAACCACAGGCAGGCCAGCCTGGAGATCAGCCCCATCACCTTC
CTGACTGCCCAGACCCTGCTGATGGACCTGGGCCAGTTCCTGCTGTTCTGCCACATCAGCAGCCACCA
GCATGATGGCATGGAGGCCTATGTGAAGGTGGACAGCTGCCCTGAGGAGCCCCAGCTGAGGATGAAGA
ACAATGAGGAGGCTGAGGACTATGATGATGACCTGACTGACTCTGAGATGGATGTGGTGAGGTTTGAT
GATGACAACAGCCCCAGCTTCATCCAGATCAGGTCTGTGGCCAAGAAGCACCCCAAGACCTGGGTGCA
CTACATTGCTGCTGAGGAGGAGGACTGGGACTATGCCCCCCTGGTGCTGGCCCCTGATGACAGGAGCT
ACAAGAGCCAGTACCTGAACAATGGCCCCCAGAGGATTGGCAGGAAGTACAAGAAGGTCAGGTTCATG
GCCTACACTGATGAAACCTTCAAGACCAGGGAGGCCATCCAGCATGAGTCTGGCATCCTGGGCCCCCT
GCTGTATGGGGAGGTGGGGGACACCCTGCTGATCATCTTCAAGAACCAGGCCAGCAGGCCCTACAACA
TCTACCCCCATGGCATCACTGATGTGAGGCCCCTGTACAGCAGGAGGCTGCCCAAGGGGGTGAAGCAC
CTGAAGGACTTCCCCATCCTGCCTGGGGAGATCTTCAAGTACAAGTGGACTGTGACTGTGGAGGATGG
CCCCACCAAGTCTGACCCCAGGTGCCTGACCAGATACTACAGCAGCTTTGTGAACATGGAGAGGGACC
TGGCCTCTGGCCTGATTGGCCCCCTGCTGATCTGCTACAAGGAGTCTGTGGACCAGAGGGGCAACCAG
ATCATGTCTGACAAGAGGAATGTGATCCTGTTCTCTGTGTTTGATGAGAACAGGAGCTGGTACCTGAC
TGAGAACATCCAGAGGTTCCTGCCCAACCCTGCTGGGGTGCAGCTGGAGGACCCTGAGTTCCAGGCCA
GCAACATCATGCACAGCATCAATGGCTATGTGTTTGACAGCCTGCAGCTGTCTGTGTGCCTGCATGAG
GTGGCCTACTGGTACATCCTGAGCATTGGGGCCCAGACTGACTTCCTGTCTGTGTTCTTCTCTGGCTA
CACCTTCAAGCACAAGATGGTGTATGAGGACACCCTGACCCTGTTCCCCTTCTCTGGGGAGACTGTGT
TCATGAGCATGGAGAACCCTGGCCTGTGGATTCTGGGCTGCCACAACTCTGACTTCAGGAACAGGGGC
ATGACTGCCCTGCTGAAAGTCTCCAGCTGTGACAAGAACACTGGGGACTACTATGAGGACAGCTATGA
GGACATCTCTGCCTACCTGCTGAGCAAGAACAATGCCATTGAGCCCAGGAGCTTCAGCCAGAATAGCA
GGCACCCCAGCACCAGGCAGAAGCAGTTCAATGCCACCACCATCCCAGAGAATACCACCCTGCAGTCT
GACCAGGAGGAGATTGACTATGATGACACCATCTCTGTGGAGATGAAGAAGGAGGACTTTGACATCTA
CGACGAGGACGAGAACCAGAGCCCCAGGAGCTTCCAGAAGAAGACCAGGCACTACTTCATTGCTGCTG
TGGAGAGGCTGTGGGACTATGGCATGAGCAGCAGCCCCCATGTGCTGAGGAACAGGGCCCAGTCTGGC
TCTGTGCCCCAGTTCAAGAAGGTGGTGTTCCAGGAGTTCACTGATGGCAGCTTCACCCAGCCCCTGTA
CAGAGGGGAGCTGAATGAGCACCTGGGCCTGCTGGGCCCCTACATCAGGGCTGAGGTGGAGGACAACA
TCATGGTGACCTTCAGGAACCAGGCCAGCAGGCCCTACAGCTTCTACAGCAGCCTGATCAGCTATGAG
GAGGACCAGAGGCAGGGGGCTGAGCCCAGGAAGAACTTTGTGAAGCCCAATGAAACCAAGACCTACTT
CTGGAAGGTGCAGCACCACATGGCCCCCACCAAGGATGAGTTTGACTGCAAGGCCTGGGCCTACTTCT
CTGATGTGGACCTGGAGAAGGATGTGCACTCTGGCCTGATTGGCCCCCTGCTGGTGTGCCACACCAAC
ACCCTGAACCCTGCCCATGGCAGGCAGGTGACTGTGCAGGAGTTTGCCCTGTTCTTCACCATCTTTGA
TGAAACCAAGAGCTGGTACTTCACTGAGAACATGGAGAGGAACTGCAGGGCCCCCTGCAACATCCAGA
TGGAGGACCCCACCTTCAAGGAGAACTACAGGTTCCATGCCATCAATGGCTACATCATGGACACCCTG
CCTGGCCTGGTGATGGCCCAGGACCAGAGGATCAGGTGGTACCTGCTGAGCATGGGCAGCAATGAGAA
CATCCACAGCATCCACTTCTCTGGCCATGTGTTCACTGTGAGGAAGAAGGAGGAGTACAAGATGGCCC
TGTACAACCTGTACCCTGGGGTGTTTGAGACTGTGGAGATGCTGCCCAGCAAGGCTGGCATCTGGAGG
GTGGAGTGCCTGATTGGGGAGCACCTGCATGCTGGCATGAGCACCCTGTTCCTGGTGTACAGCAACAA
GTGCCAGACCCCCCTGGGCATGGCCTCTGGCCACATCAGGGACTTCCAGATCACTGCCTCTGGCCAGT
ATGGCCAGTGGGCCCCCAAGCTGGCCAGGCTGCACTACTCTGGCAGCATCAATGCCTGGAGCACCAAG
GAGCCCTTCAGCTGGATCAAGGTGGACCTGCTGGCCCCCATGATCATCCATGGCATCAAGACCCAGGG
GGCCAGGCAGAAGTTCAGCAGCCTGTACATCAGCCAGTTCATCATCATGTACAGCCTGGATGGCAAGA
AGTGGCAGACCTACAGGGGCAACAGCACTGGCACCCTGATGGTGTTCTTTGGCAATGTGGACAGCTCT
GGCATCAAGCACAACATCTTCAACCCCCCCATCATTGCCAGATACATCAGGCTGCACCCCACCCACTA
CAGCATCAGGAGCACCCTGAGGATGGAGCTGATGGGCTGTGACCTGAACAGCTGCAGCATGCCCCTGG
GCATGGAGAGCAAGGCCATCTCTGATGCCCAGATCACTGCCAGCAGCTACTTCACCAACATGTTTGCC
ACCTGGAGCCCCAGCAAGGCCAGGCTGCACCTGCAGGGCAGGAGCAATGCCTGGAGGCCCCAGGTCAA
CAACCCCAAGGAGTGGCTGCAGGTGGACTTCCAGAAGACCATGAAGGTGACTGGGGTGACCACCCAGG
GGGTGAAGAGCCTGCTGACCAGCATGTATGTGAAGGAGTTCCTGATCAGCAGCAGCCAGGATGGCCAC
CAGTGGACCCTGTTCTTCCAGAATGGCAAGGTGAAGGTGTTCCAGGGCAACCAGGACAGCTTCACCCC
TGTGGTGAACAGCCTGGACCCCCCCCTGCTGACCAGATACCTGAGGATTCACCCCCAGAGCTGGGTGC
ACCAGATTGCCCTGAGGATGGAGGTGCTGGGCTGTGAGGCCCAGGACCTGTACTGATTAATTAAGAGC
ATCTTACCGCCATTTATTCCCATATTTGTTCTGTTTTTCTTGATTTGGGTATACATTTAAATGTTAAT
AAAACAAAATGGTGGGGCAATCATTTACATTTTTAGGGATATGTAATTACTAGTTCAGGTGTATTGCC
ACAAGACAAACATGTTAAGAAACTTTCCCGTTATTTACGCTCTGTTCCTGTTAATCAACCTCTGGATT
ACAAAATTTGTGAAAGATTGACTGATATTCTTAACTATGTTGCTCCTTTTACGCTGTGTGGATATGCT
GCTTTATAGCCTCTGTATCTAGCTATTGCTTCCCGTACGGCTTTCGTTTTCTCCTCCTTGTATAAATC
CTGGTTGCTGTCTCTTTTAGAGGAGTTGTGGCCCGTTGTCCGTCAACGTGGCGTGGTGTGCTCTGTGT
TTGCTGACGCAACCCCCACTGGCTGGGGCATTGCCACCACCTGTCAACTCCTTTCTGGGACTTTCGCT
TTCCCCCTCCCGATCGCCACGGCAGAACTCATCGCCGCCTGCCTTGCCCGCTGCTGGACAGGGGCTAG
GTTGCTGGGCACTGATAATTCCGTGGTGTTGTCTGTGCCTTCTAGTTGCCAGCCATCTGTTGTTTGCC
CCTCCCCCGTGCCTTCCTTGACCCTGGAAGGTGCCACTCCCACTGTCCTTTCCTAATAAAATGAGGAA
ATTGCATCGCATTGTCTGAGTAGGTGTCATTCTATTCTGGGGGGTGGGGTGGGGCAGGACAGCAAGGG
GGAGGATTGGGAAGACAATAGCAGGCATGCTGGGGATGCGGTGGGCTCTATGGCTCTAGAGCATGGCT
ACGTAGATAAGTAGCATGGCGGGTTAATCATTAACTACACCTGCAGGAGGAACCCCTAGTGATGGAGT
TGGCCACTCCCTCTCTGCGCGCTCGCTCGCTCACTCGAGACCACCGGTTTACAGGATAGTCTACTCCC
AGTGTCCACATGGTTAGGACCGAGGGGCTTTGGGTTTAGACACTGGCGACTTCCGCTTCCTCGCTCAC
TGACTCGCTGCGCTCGGTCGTTCGGCTGCGGCGAGCGGTATCAGCTCACTCAAAGGCGGTAATACGGT
TATCCACAGAATCAGGGGATAACGCAGGAAAGAACATGTGAGCAAAAGGCCAGCAAAAGGCCAGGAAC
CGTAAAAAGGCCGCGTTGCTGGCGTTTTTCCATAGGCTCCGCCCCCCTGACGAGCATCACAAAAATCG
ACGCTCAAGTCAGAGGTGGCGAAACCCGACAGGACTATAAAGATACCAGGCGTTTCCCCCTGGAAGCT
CCCTCGTGCGCTCTCCTGTTCCGACCCTGCCGCTTACCGGATACCTGTCCGCCTTTCTCCCTTCGGGA
AGCGTGGCGCTTTCTCATAGCTCACGCTGTAGGTATCTCAGTTCGGTGTAGGTCGTTCGCTCCAAGCT
GGGCTGTGTGCACGAACCCCCCGTTCAGCCCGACCGCTGCGCCTTATCCGGTAACTATCGTCTTGAGT
CCAACCCGGTAAGACACGACTTATCGCCACTGGCAGCAGCCACTGGTAACAGGATTAGCAGAGCGAGG
TATGTAGGCGGTGCTACAGAGTTCTTGAAGTGGTGGCCTAACTACGGCTACACTAGAAGAACAGTATT
TGGTATCTGCGCTCTGCTGAAGCCAGTTACCTTCGGAAAAAGAGTTGGTAGCTCTTGATCCGGCAAAC
AAACCACCGCTGGTAGCGGTGGTTTTTTTGTTTGCAAGCAGCAGATTACGCGCAGAAAAAAAGGATCT
CAAGAAGATCCTTTGATCTTTTCTACGGGGTCTGACGCTCAGTGGAACGAAAACTCACGTTAAGGGAT
TTTGGTCATGAGATTATCAAAAAGGATCTTCACCTAGATCCTTTTAAATTAAAAATGAAGTTTTAAAT
CAATCTAAAGTATATATGAGTAAACTTGGTCTGACAGTTACCAATGCTTAATCAGTGAGGCACCTATC
TCAGCGATCTGTCTATTTCGTTCATCCATAGTTGCCTGACTCCCCGTCGTGTAGATAACTACGATACG
GGAGGGCTTACCATCTGGCCCCAGTGCTGCAATGATACCGCGAGACCCACGCTCACCGGCTCCAGATT
TATCAGCAATAAACCAGCCAGCCGGAAGGGCCGAGCGCAGAAGTGGTCCTGCAACTTTATCCGCCTCC
ATCCAGTCTATTAATTGTTGCCGGGAAGCTAGAGTAAGTAGTTCGCCAGTTAATAGTTTGCGCAACGT
TGTTGCCATTGCTACAGGCATCGTGGTGTCACGCTCGTCGTTTGGTATGGCTTCATTCAGCTCCGGTT
CCCAACGATCAAGGCGAGTTACATGATCCCCCATGTTGTGCAAAAAAGCGGTTAGCTCCTTCGGTCCT
CCGATCGTTGTCAGAAGTAAGTTGGCCGCAGTGTTATCACTCATGGTTATGGCAGCACTGCATAATTC
TCTTACTGTCATGCCATCCGTAAGATGCTTTTCTGTGACTGGTGAGTACTCAACCAAGTCATTCTGAG
AATAGTGTATGCGGCGACCGAGTTGCTCTTGCCCGGCGTCAATACGGGATAATACCGCGCCACATAGC
AGAACTTTAAAAGTGCTCATCATTGGAAAACGTTCTTCGGGGCGAAAACTCTCAAGGATCTTACCGCT
GTTGAGATCCAGTTCGATGTAACCCACTCGTGCACCCAACTGATCTTCAGCATCTTTTACTTTCACCA
GCGTTTCTGGGTGAGCAAAAACAGGAAGGCAAAATGCCGCAAAAAAGGGAATAAGGGCGACACGGAAA
TGTTGAATACTCATACTCTTCCTTTTTCAATATTATTGAAGCATTTATCAGGGTTATTGTCTCATGAG
CGGATACATATTTGAATGTATTTAGAAAAATAAACAAAT

### Example 2: Rolling Circle Amplification (RCA)-Dependent ceDNA Production Enabled Scalable and High-Purity Cell-Free Synthesis of Single-Stranded Closed-Ended DNA Vectors (ssDNA)

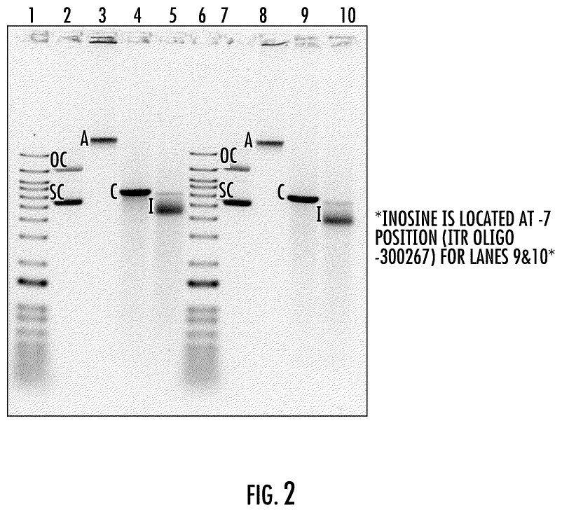

In this Example, ssDNA was produced from ceDNA manufactured using rolling circle amplification (RCA) of the plasmid template. In this way small amounts of plasmid precursor were used to generate large amounts of high purity substrate. Implementation of RCA enabled scalable cell free synthesis of single-stranded closed-ended DNA vectors (ssDNA). FIG. 2 shows the steps in producing ssDNA from RCA-plasmid precursor via a ceDNA intermediate.

Rolling Circle Amplification of ceDNA

Briefly, RCA amplification of plasmid SP-704 was performed by mixing 500 ng of template and a single primer (GGTCAAG*T*G; * indicates phosphorothioate bonds) that served as both a forward and reverse primer in the presence of 1× EquiPhi29 reaction buffer. The primer was annealed to the plasmid by heating the mixture to 95° C. for 5 mins and snap chilling on ice. The following was added to the annealed mixture for a final reaction volume of 0.5 mL: dNTPs (4 mM final concentration), Dithiothreitol (DTT) (1 mM final concentration), 25 units of EquiPhi29 DNA polymerase, 0.25 units of yeast inorganic pyrophosphatase, 50 microliters of 10× EquiPhi29 reaction buffer. The reaction was incubated at 30° C. for 18 h. The RCA reaction was heat inactivated at 65° C. for 20 mins. Heat-inactivated RCA reaction containing total amplified plasmid was converted to ceDNA as follows. Synthetic oligonucleotides containing ends compatible with subsequent ligation to digested RCA product were annealed by heating to 95° C. and snap chilling on ice. The following was added to the heat-inactivated RCA reaction in the presence of 1× T4 ligase buffer (50 mM Tris-HCl, 10 mM MgCl₂, 1 mM ATP, 10 mM DTT): annealed oligonucleotides, BsaI restriction endonuclease (1 unit/microgram of amplified DNA). T4 DNA ligase (5 units/microgram of amplified DNA) was added to the reaction and incubated for 17 h at 37° C. The reaction was heat inactivated at 65° C. for 20 mins. T5 exonuclease was added (4 units/microgram of amplified DNA) to the reaction. Ethylenediaminetetraacetic acid (EDTA) was added to a final concentration of 50 mM to terminate the reaction. The resulting ceDNA was purified using the DNA Clean & Concentrator system (Zymo Research) and eluted in nuclease-free water.

Conversion of ceDNA to Single-Stranded DNA (SSD)

ceDNA was converted to SSD as follows. 1.5 micrograms of purified ceDNA was treated with 1 unit of Nb.BbvCI per microgram of DNA for 2 h at 37° C. in the presence of 1× NEBuffer 4 (50 mM Potassium acetate, 20 mM Tris-Acetate, 10 mM Magnesium acetate, 1 mM DTT). Subsequently, 0.3 Units of T7 exonuclease were added per microgram of DNA and incubated at 37° C. for 30 mins. EDTA was added to a final concentration of 50 mM to terminate the reaction. The resulting products were purified via the DNA Clean & Concentrator system (Zymo Research) and eluted in nuclease-free water.

FIG. 2 shows single-stranded DNA (ssDNA or SSD) synthesis via rolling circle amplification and enzymatic synthesis. Plasmid template (lane 2 and 7) was subject to rolling circle amplification to produce intermediate dsDNA product “A” (lane 3). This intermediate product was subject to enzymatic synthesis to produce closed-ended DNA (ceDNA) product “C” (lane 4 and 9). ceDNA was further processed using either Nb.BbvCI (lane 5) or Endonuclease V (lane 10) to produce ssDNA “I”. oc, open circular plasmid; sc, supercoiled plasmid; A, amplification product; C, ceDNA; I, ssDNA.

The ssDNA product has PS bonds near the 5′ and 3′ ends, and was derived from synthetic double-stranded ceDNA generated rapidly from amplified (RAMP) KAN plasmid.

TABLE 7

| Lane | | | | ssDNA (if | plasmid (if |
|---|---|---|---|---|---|
| number | Description | Reaction | ceDNA | applicable) | applicable) |
| 1 | Size marker | NA | NA | NA | NA |
| 2 | Plasmid precursor | NA | NA | NA | 704 |
| 3 | Amplified Plasmid | Amplification (RCA) | NA | NA | 704 |
| 4 | ceDNA | RES | 655 | NA | |
| 5 | ssDNA generated | ssDNA synthesis | 655 | 041 | |
| | with Nb.BbvCl | | | | |
| 6 | Size marker | NA | NA | NA | NA |
| 7 | Plasmid precursor | NA | NA | NA | 704 |
| 8 | Amplified Plasmid | Amplification (RCA) | NA | NA | 704 |
| 9 | ceDNA | RES | 656 | NA | |
| 10 | ssDNA generated with | ssDNA synthesis | 656 | 042 | |
| | Endonuclease V | | | | |

704 (Lanes 2, 3, 7, 8; 8180 bp; hFVIII ORF 806-5197)
GGTCAAGTGCCTAAGCTCACTCGAGGGTCTCGTGAGCGAGCGAGCGCGCAGAGAGGGAGTGGCCAAGC
TGAGGCTCCATCACTAGGGGTTCCTTGTAGTTAATGATTAACCCGCCATGCTACTTATCGCGGCCGCG
GGGGAGGCTGCTGGTGAATATTAACCAAGGTCACCCCAGTTATCGGAGGAGCAAACAGGGGCTAAGTC
CACCGGGGGAGGCTGCTGGTGAATATTAACCAAGGTCACCCCAGTTATCGGAGGAGCAAACAGGGGCT
AAGTCCACCGGGGGAGGCTGCTGGTGAATATTAACCAAGGTCACCCCAGTTATCGGAGGAGCAAACAG
GGGCTAAGTCCACGGTACCCACTGGGAGGATGTTGAGTAAGATGGAAAACTACTGATGACCCTTGCAG
AGACAGAGTATTAGGACATGTTTGAACAGGGGCCGGGCGATCAGCAGGTAGCTCTAGAGGATCCCCGT
CTGTCTGCACATTTCGTAGAGCGAGTGTTCCGATACTCTAATCTCCCTAGGCAAGGTTCATATTTGTG
TAGGTTACTTATTCTCCTTTTGTTGACTAAGTCAATAATCAGAATCAGCAGGTTTGGAGTCAGCTTGG
CAGGGATCAGCAGCCTGGGTTGGAAGGAGGGGGTATAAAAGCCCCTTCACCAGGAGAAGCCGTCACAC
AGATCCACAAGCTCCTGAAGAGGTAAGGGTTTAAGGGATGGTTGGTTGGTGGGGTATTAATGTTTAAT
TACCTGGAGCACCTGCCTGAAATCACTTTTTTTCAGGTTGGTTTAAACGCCGCCACCATGCAGATTGA
GCTGAGCACCTGCTTCTTCCTGTGCCTGCTGAGGTTCTGCTTCTCTGCCACCAGGAGATACTACCTGG
GGGCTGTGGAGCTGAGCTGGGACTACATGCAGTCTGACCTGGGGGAGCTGCCTGTGGATGCCAGGTTC
CCCCCCAGAGTGCCCAAGAGCTTCCCCTTCAACACCTCTGTGGTGTACAAGAAGACCCTGTTTGTGGA
GTTCACTGACCACCTGTTCAACATTGCCAAGCCCAGGCCCCCCTGGATGGGCCTGCTGGGCCCCACCA
TCCAGGCTGAGGTGTATGACACTGTGGTGATCACCCTGAAGAACATGGCCAGCCACCCTGTGAGCCTG
CATGCTGTGGGGGTGAGCTACTGGAAGGCCTCTGAGGGGGCTGAGTATGATGACCAGACCAGCCAGAG
GGAGAAGGAGGATGACAAGGTGTTCCCTGGGGGCAGCCACACCTATGTGTGGCAGGTGCTGAAGGAGA
ATGGCCCCATGGCCTCTGACCCCCTGTGCCTGACCTACAGCTACCTGAGCCATGTGGACCTGGTGAAG
GACCTGAACTCTGGCCTGATTGGGGCCCTGCTGGTGTGCAGGGAGGGCAGCCTGGCCAAGGAGAAGAC
CCAGACCCTGCACAAGTTCATCCTGCTGTTTGCTGTGTTTGATGAGGGCAAGAGCTGGCACTCTGAAA
CCAAGAACAGCCTGATGCAGGACAGGGATGCTGCCTCTGCCAGGGCCTGGCCCAAGATGCACACTGTG
AATGGCTATGTGAACAGGAGCCTGCCTGGCCTGATTGGCTGCCACAGGAAGTCTGTGTACTGGCATGT
GATTGGCATGGGCACCACCCCTGAGGTGCACAGCATCTTCCTGGAGGGCCACACCTTCCTGGTCAGGA
ACCACAGGCAGGCCAGCCTGGAGATCAGCCCCATCACCTTCCTGACTGCCCAGACCCTGCTGATGGAC
CTGGGCCAGTTCCTGCTGTTCTGCCACATCAGCAGCCACCAGCATGATGGCATGGAGGCCTATGTGAA
GGTGGACAGCTGCCCTGAGGAGCCCCAGCTGAGGATGAAGAACAATGAGGAGGCTGAGGACTATGATG
ATGACCTGACTGACTCTGAGATGGATGTGGTGAGGTTTGATGATGACAACAGCCCCAGCTTCATCCAG
ATCAGGTCTGTGGCCAAGAAGCACCCCAAGACCTGGGTGCACTACATTGCTGCTGAGGAGGAGGACTG
GGACTATGCCCCCCTGGTGCTGGCCCCTGATGACAGGAGCTACAAGAGCCAGTACCTGAACAATGGCC
CCCAGAGGATTGGCAGGAAGTACAAGAAGGTCAGGTTCATGGCCTACACTGATGAAACCTTCAAGACC
AGGGAGGCCATCCAGCATGAGTCTGGCATCCTGGGCCCCCTGCTGTATGGGGAGGTGGGGGACACCCT
GCTGATCATCTTCAAGAACCAGGCCAGCAGGCCCTACAACATCTACCCCCATGGCATCACTGATGTGA
GGCCCCTGTACAGCAGGAGGCTGCCCAAGGGGGTGAAGCACCTGAAGGACTTCCCCATCCTGCCTGGG
GAGATCTTCAAGTACAAGTGGACTGTGACTGTGGAGGATGGCCCCACCAAGTCTGACCCCAGGTGCCT
GACCAGATACTACAGCAGCTTTGTGAACATGGAGAGGGACCTGGCCTCTGGCCTGATTGGCCCCCTGC
TGATCTGCTACAAGGAGTCTGTGGACCAGAGGGGCAACCAGATCATGTCTGACAAGAGGAATGTGATC
CTGTTCTCTGTGTTTGATGAGAACAGGAGCTGGTACCTGACTGAGAACATCCAGAGGTTCCTGCCCAA
CCCTGCTGGGGTGCAGCTGGAGGACCCTGAGTTCCAGGCCAGCAACATCATGCACAGCATCAATGGCT
ATGTGTTTGACAGCCTGCAGCTGTCTGTGTGCCTGCATGAGGTGGCCTACTGGTACATCCTGAGCATT
GGGGCCCAGACTGACTTCCTGTCTGTGTTCTTCTCTGGCTACACCTTCAAGCACAAGATGGTGTATGA
GGACACCCTGACCCTGTTCCCCTTCTCTGGGGAGACTGTGTTCATGAGCATGGAGAACCCTGGCCTGT
GGATTCTGGGCTGCCACAACTCTGACTTCAGGAACAGGGGCATGACTGCCCTGCTGAAAGTCTCCAGC
TGTGACAAGAACACTGGGGACTACTATGAGGACAGCTATGAGGACATCTCTGCCTACCTGCTGAGCAA
GAACAATGCCATTGAGCCCAGGAGCTTCAGCCAGAATAGCAGGCACCCCAGCACCAGGCAGAAGCAGT
TCAATGCCACCACCATCCCAGAGAATACCACCCTGCAGTCTGACCAGGAGGAGATTGACTATGATGAC
ACCATCTCTGTGGAGATGAAGAAGGAGGACTTTGACATCTACGACGAGGACGAGAACCAGAGCCCCAG
GAGCTTCCAGAAGAAGACCAGGCACTACTTCATTGCTGCTGTGGAGAGGCTGTGGGACTATGGCATGA
GCAGCAGCCCCCATGTGCTGAGGAACAGGGCCCAGTCTGGCTCTGTGCCCCAGTTCAAGAAGGTGGTG
TTCCAGGAGTTCACTGATGGCAGCTTCACCCAGCCCCTGTACAGAGGGGAGCTGAATGAGCACCTGGG
CCTGCTGGGCCCCTACATCAGGGCTGAGGTGGAGGACAACATCATGGTGACCTTCAGGAACCAGGCCA
GCAGGCCCTACAGCTTCTACAGCAGCCTGATCAGCTATGAGGAGGACCAGAGGCAGGGGGCTGAGCCC
AGGAAGAACTTTGTGAAGCCCAATGAAACCAAGACCTACTTCTGGAAGGTGCAGCACCACATGGCCCC
CACCAAGGATGAGTTTGACTGCAAGGCCTGGGCCTACTTCTCTGATGTGGACCTGGAGAAGGATGTGC
ACTCTGGCCTGATTGGCCCCCTGCTGGTGTGCCACACCAACACCCTGAACCCTGCCCATGGCAGGCAG
GTGACTGTGCAGGAGTTTGCCCTGTTCTTCACCATCTTTGATGAAACCAAGAGCTGGTACTTCACTGA
GAACATGGAGAGGAACTGCAGGGCCCCCTGCAACATCCAGATGGAGGACCCCACCTTCAAGGAGAACT
ACAGGTTCCATGCCATCAATGGCTACATCATGGACACCCTGCCTGGCCTGGTGATGGCCCAGGACCAG
AGGATCAGGTGGTACCTGCTGAGCATGGGCAGCAATGAGAACATCCACAGCATCCACTTCTCTGGCCA
TGTGTTCACTGTGAGGAAGAAGGAGGAGTACAAGATGGCCCTGTACAACCTGTACCCTGGGGTGTTTG
AGACTGTGGAGATGCTGCCCAGCAAGGCTGGCATCTGGAGGGTGGAGTGCCTGATTGGGGAGCACCTG
CATGCTGGCATGAGCACCCTGTTCCTGGTGTACAGCAACAAGTGCCAGACCCCCCTGGGCATGGCCTC
TGGCCACATCAGGGACTTCCAGATCACTGCCTCTGGCCAGTATGGCCAGTGGGCCCCCAAGCTGGCCA
GGCTGCACTACTCTGGCAGCATCAATGCCTGGAGCACCAAGGAGCCCTTCAGCTGGATCAAGGTGGAC
CTGCTGGCCCCCATGATCATCCATGGCATCAAGACCCAGGGGGCCAGGCAGAAGTTCAGCAGCCTGTA
CATCAGCCAGTTCATCATCATGTACAGCCTGGATGGCAAGAAGTGGCAGACCTACAGGGGCAACAGCA
CTGGCACCCTGATGGTGTTCTTTGGCAATGTGGACAGCTCTGGCATCAAGCACAACATCTTCAACCCC
CCCATCATTGCCAGATACATCAGGCTGCACCCCACCCACTACAGCATCAGGAGCACCCTGAGGATGGA
GCTGATGGGCTGTGACCTGAACAGCTGCAGCATGCCCCTGGGCATGGAGAGCAAGGCCATCTCTGATG
CCCAGATCACTGCCAGCAGCTACTTCACCAACATGTTTGCCACCTGGAGCCCCAGCAAGGCCAGGCTG
CACCTGCAGGGCAGGAGCAATGCCTGGAGGCCCCAGGTCAACAACCCCAAGGAGTGGCTGCAGGTGGA
CTTCCAGAAGACCATGAAGGTGACTGGGGTGACCACCCAGGGGGTGAAGAGCCTGCTGACCAGCATGT
ATGTGAAGGAGTTCCTGATCAGCAGCAGCCAGGATGGCCACCAGTGGACCCTGTTCTTCCAGAATGGC
AAGGTGAAGGTGTTCCAGGGCAACCAGGACAGCTTCACCCCTGTGGTGAACAGCCTGGACCCCCCCCT
GCTGACCAGATACCTGAGGATTCACCCCCAGAGCTGGGTGCACCAGATTGCCCTGAGGATGGAGGTGC
TGGGCTGTGAGGCCCAGGACCTGTACTGATTAATTAAGAGCATCTTACCGCCATTTATTCCCATATTT
GTTCTGTTTTTCTTGATTTGGGTATACATTTAAATGTTAATAAAACAAAATGGTGGGGCAATCATTTA
CATTTTTAGGGATATGTAATTACTAGTTCAGGTGTATTGCCACAAGACAAACATGTTAAGAAACTTTC
CCGTTATTTACGCTCTGTTCCTGTTAATCAACCTCTGGATTACAAAATTTGTGAAAGATTGACTGATA
TTCTTAACTATGTTGCTCCTTTTACGCTGTGTGGATATGCTGCTTTATAGCCTCTGTATCTAGCTATT
GCTTCCCGTACGGCTTTCGTTTTCTCCTCCTTGTATAAATCCTGGTTGCTGTCTCTTTTAGAGGAGTT
GTGGCCCGTTGTCCGTCAACGTGGCGTGGTGTGCTCTGTGTTTGCTGACGCAACCCCCACTGGCTGGG
GCATTGCCACCACCTGTCAACTCCTTTCTGGGACTTTCGCTTTCCCCCTCCCGATCGCCACGGCAGAA
CTCATCGCCGCCTGCCTTGCCCGCTGCTGGACAGGGGCTAGGTTGCTGGGCACTGATAATTCCGTGGT
GTTGTCTGTGCCTTCTAGTTGCCAGCCATCTGTTGTTTGCCCCTCCCCCGTGCCTTCCTTGACCCTGG
AAGGTGCCACTCCCACTGTCCTTTCCTAATAAAATGAGGAAATTGCATCGCATTGTCTGAGTAGGTGT
CATTCTATTCTGGGGGGTGGGGTGGGGCAGGACAGCAAGGGGGAGGATTGGGAAGACAATAGCAGGCA
TGCTGGGGATGCGGTGGGCTCTATGGCTCTAGAGCATGGCTACGTAGATAAGTAGCATGGCGGGTTAA
TCATTAACTACACCTGCAGGAGGAACCCCTAGTGATGGAGTTGGCCACTCCCTCTCTGCGCGCTCGCT
CGCTCACTCGAGACCACCGGTGAGCTTAGGCACTTGACCGGCAAGCCCTTGTGGACGTCTACTCCCAG
TGTCCACATGGTTAGGACCGAGGGGCTTTGGGTTTAGACACTGGCGACTTCCGCTTCCTCGCTCACTG
ACTCGCTGCGCTCGGTCGTTCGGCTGCGGCGAGCGGTATCAGCTCACTCAAAGGCGGTAATACGGTTA
TCCACAGAATCAGGGGATAACGCAGGAAAGAACATGTGAGCAAAAGGCCAGCAAAAGGCCAGGAACCG
TAAAAAGGCCGCGTTGCTGGCGTTTTTCCATAGGCTCCGCCCCCCTGACGAGCATCACAAAAATCGAC
GCTCAAGTCAGAGGTGGCGAAACCCGACAGGACTATAAAGATACCAGGCGTTTCCCCCTGGAAGCTCC
CTCGTGCGCTCTCCTGTTCCGACCCTGCCGCTTACCGGATACCTGTCCGCCTTTCTCCCTTCGGGAAG
CGTGGCGCTTTCTCATAGCTCACGCTGTAGGTATCTCAGTTCGGTGTAGGTCGTTCGCTCCAAGCTGG
GCTGTGTGCACGAACCCCCCGTTCAGCCCGACCGCTGCGCCTTATCCGGTAACTATCGTCTTGAGTCC
AACCCGGTAAGACACGACTTATCGCCACTGGCAGCAGCCACTGGTAACAGGATTAGCAGAGCGAGGTA
TGTAGGCGGTGCTACAGAGTTCTTGAAGTGGTGGCCTAACTACGGCTACACTAGAAGAACAGTATTTG
GTATCTGCGCTCTGCTGAAGCCAGTTACCTTCGGAAAAAGAGTTGGTAGCTCTTGATCCGGCAAACAA
ACCACCGCTGGTAGCGGTGGTTTTTTTGTTTGCAAGCAGCAGATTACGCGCAGAAAAAAAGGATCTCA
AGAAGATCCTTTGATCTTTTCTACGGGGTCTGACGCTCAGTGGAACGAAAACTCACGTTAAGGGATTT
TGGTCATGAGATTATCAAAAAGGATCTTCACCTAGATCCTTTTAAATTAAAAATGAAGTTTTAAATCA
ATCTAAAGTATATATGAGTAAACTTGGTCTGACAGACCGCGAGACCTTAGAAAAACTCATCGAGCATC
AAATGAAACTGCAATTTATTCATATCAGGATTATCAATACCATATTTTTGAAAAAGCCGTTTCTGTAA
TGAAGGAGAAAACTCACCGAGGCAGTTCCATAGGATGGCAAGATCCTGGTATCGGTCTGCGATTCCGA
CTCGTCCAACATCAATACAACCTATTAATTTCCCCTCGTCAAAAATAAGGTTATCAAGTGAGAAATCA
CCATGAGTGACGACTGAATCCGGTGAGAATGGCAAAAGTTTATGCATTTCTTTCCAGACTTGTTCAAC
AGGCCAGCCATTACGCTCGTCATCAAAATCACTCGCATCAACCAAACCGTTATTCATTCGTGATTGCG
CCTGAGCGAGGCGAAATACGCGATCGCTGTTAAAAGGACAATTACAAACAGGAATCGAATGCAACCGG
CGCAGGAACACTGCCAGCGCATCAACAATATTTTCACCTGAATCAGGATATTCTTCTAATACCTGGAA
TGCTGTTTTTCCGGGGATCGCAGTGGTGAGTAACCATGCATCATCAGGAGTACGGATAAAATGCTTGA
TGGTCGGAAGTGGCATAAATTCCGTCAGCCAGTTTAGTCTGACCATCTCATCTGTAACATCATTGGCA
ACGCTACCTTTGCCATGTTTCAGAAACAACTCTGGCGCATCGGGCTTCCCATACAAGCGATAGATTGT
CGCACCTGATTGCCCGACATTATCGCGAGCCCATTTATACCCATATAAATCAGCATCCATGTTGGAAT
TTAATCGCGGCCTCGACGTTTCCCGTTGAATATGGCTCATAACACCCCTTGTATTACTGTTTATGTAA
GCAGACAGTTTTATTGTTCATGATGATATATTTTTATCTTGTGCAATGTAACATCAGAGATTTTGAGA
CACCGCACATTTCCCCGAAAAGTGCCACCTGACGTCTTTCGTCAGATCACCGGGTTGTTCCATATCAT
CGTGTCCACAAGGGCTTGCC
655 (Lanes 4-5; 6214 bp; hFVIII ORF 835-5226)
CTCACTGAGGCCGCCCGGGCAAAGCCCGGGCGTCGGGCGACCTTTGGTCGCCCGGCCTCAGTGAGCGA
GCGAGCGCGCAGAGAGGGAGTGGCCAAGCTGAGGCTCCATCACTAGGGGTTCCTTGTAGTTAATGATT
AACCCGCCATGCTACTTATCGCGGCCGCGGGGGAGGCTGCTGGTGAATATTAACCAAGGTCACCCCAG
TTATCGGAGGAGCAAACAGGGGCTAAGTCCACCGGGGGAGGCTGCTGGTGAATATTAACCAAGGTCAC
CCCAGTTATCGGAGGAGCAAACAGGGGCTAAGTCCACCGGGGGAGGCTGCTGGTGAATATTAACCAAG
GTCACCCCAGTTATCGGAGGAGCAAACAGGGGCTAAGTCCACGGTACCCACTGGGAGGATGTTGAGTA
AGATGGAAAACTACTGATGACCCTTGCAGAGACAGAGTATTAGGACATGTTTGAACAGGGGCCGGGCG
ATCAGCAGGTAGCTCTAGAGGATCCCCGTCTGTCTGCACATTTCGTAGAGCGAGTGTTCCGATACTCT
AATCTCCCTAGGCAAGGTTCATATTTGTGTAGGTTACTTATTCTCCTTTTGTTGACTAAGTCAATAAT
CAGAATCAGCAGGTTTGGAGTCAGCTTGGCAGGGATCAGCAGCCTGGGTTGGAAGGAGGGGGTATAAA
AGCCCCTTCACCAGGAGAAGCCGTCACACAGATCCACAAGCTCCTGAAGAGGTAAGGGTTTAAGGGAT
GGTTGGTTGGTGGGGTATTAATGTTTAATTACCTGGAGCACCTGCCTGAAATCACTTTTTTTCAGGTT
GGTTTAAACGCCGCCACCATGCAGATTGAGCTGAGCACCTGCTTCTTCCTGTGCCTGCTGAGGTTCTG
CTTCTCTGCCACCAGGAGATACTACCTGGGGGCTGTGGAGCTGAGCTGGGACTACATGCAGTCTGACC
TGGGGGAGCTGCCTGTGGATGCCAGGTTCCCCCCCAGAGTGCCCAAGAGCTTCCCCTTCAACACCTCT
GTGGTGTACAAGAAGACCCTGTTTGTGGAGTTCACTGACCACCTGTTCAACATTGCCAAGCCCAGGCC
CCCCTGGATGGGCCTGCTGGGCCCCACCATCCAGGCTGAGGTGTATGACACTGTGGTGATCACCCTGA
AGAACATGGCCAGCCACCCTGTGAGCCTGCATGCTGTGGGGGTGAGCTACTGGAAGGCCTCTGAGGGG
GCTGAGTATGATGACCAGACCAGCCAGAGGGAGAAGGAGGATGACAAGGTGTTCCCTGGGGGCAGCCA
CACCTATGTGTGGCAGGTGCTGAAGGAGAATGGCCCCATGGCCTCTGACCCCCTGTGCCTGACCTACA
GCTACCTGAGCCATGTGGACCTGGTGAAGGACCTGAACTCTGGCCTGATTGGGGCCCTGCTGGTGTGC
AGGGAGGGCAGCCTGGCCAAGGAGAAGACCCAGACCCTGCACAAGTTCATCCTGCTGTTTGCTGTGTT
TGATGAGGGCAAGAGCTGGCACTCTGAAACCAAGAACAGCCTGATGCAGGACAGGGATGCTGCCTCTG
CCAGGGCCTGGCCCAAGATGCACACTGTGAATGGCTATGTGAACAGGAGCCTGCCTGGCCTGATTGGC
TGCCACAGGAAGTCTGTGTACTGGCATGTGATTGGCATGGGCACCACCCCTGAGGTGCACAGCATCTT
CCTGGAGGGCCACACCTTCCTGGTCAGGAACCACAGGCAGGCCAGCCTGGAGATCAGCCCCATCACCT
TCCTGACTGCCCAGACCCTGCTGATGGACCTGGGCCAGTTCCTGCTGTTCTGCCACATCAGCAGCCAC
CAGCATGATGGCATGGAGGCCTATGTGAAGGTGGACAGCTGCCCTGAGGAGCCCCAGCTGAGGATGAA
GAACAATGAGGAGGCTGAGGACTATGATGATGACCTGACTGACTCTGAGATGGATGTGGTGAGGTTTG
ATGATGACAACAGCCCCAGCTTCATCCAGATCAGGTCTGTGGCCAAGAAGCACCCCAAGACCTGGGTG
CACTACATTGCTGCTGAGGAGGAGGACTGGGACTATGCCCCCCTGGTGCTGGCCCCTGATGACAGGAG
CTACAAGAGCCAGTACCTGAACAATGGCCCCCAGAGGATTGGCAGGAAGTACAAGAAGGTCAGGTTCA
TGGCCTACACTGATGAAACCTTCAAGACCAGGGAGGCCATCCAGCATGAGTCTGGCATCCTGGGCCCC
CTGCTGTATGGGGAGGTGGGGGACACCCTGCTGATCATCTTCAAGAACCAGGCCAGCAGGCCCTACAA
CATCTACCCCCATGGCATCACTGATGTGAGGCCCCTGTACAGCAGGAGGCTGCCCAAGGGGGTGAAGC
ACCTGAAGGACTTCCCCATCCTGCCTGGGGAGATCTTCAAGTACAAGTGGACTGTGACTGTGGAGGAT
GGCCCCACCAAGTCTGACCCCAGGTGCCTGACCAGATACTACAGCAGCTTTGTGAACATGGAGAGGGA
CCTGGCCTCTGGCCTGATTGGCCCCCTGCTGATCTGCTACAAGGAGTCTGTGGACCAGAGGGGCAACC
AGATCATGTCTGACAAGAGGAATGTGATCCTGTTCTCTGTGTTTGATGAGAACAGGAGCTGGTACCTG
ACTGAGAACATCCAGAGGTTCCTGCCCAACCCTGCTGGGGTGCAGCTGGAGGACCCTGAGTTCCAGGC
CAGCAACATCATGCACAGCATCAATGGCTATGTGTTTGACAGCCTGCAGCTGTCTGTGTGCCTGCATG
AGGTGGCCTACTGGTACATCCTGAGCATTGGGGCCCAGACTGACTTCCTGTCTGTGTTCTTCTCTGGC
TACACCTTCAAGCACAAGATGGTGTATGAGGACACCCTGACCCTGTTCCCCTTCTCTGGGGAGACTGT
GTTCATGAGCATGGAGAACCCTGGCCTGTGGATTCTGGGCTGCCACAACTCTGACTTCAGGAACAGGG
GCATGACTGCCCTGCTGAAAGTCTCCAGCTGTGACAAGAACACTGGGGACTACTATGAGGACAGCTAT
GAGGACATCTCTGCCTACCTGCTGAGCAAGAACAATGCCATTGAGCCCAGGAGCTTCAGCCAGAATAG
CAGGCACCCCAGCACCAGGCAGAAGCAGTTCAATGCCACCACCATCCCAGAGAATACCACCCTGCAGT
CTGACCAGGAGGAGATTGACTATGATGACACCATCTCTGTGGAGATGAAGAAGGAGGACTTTGACATC
TACGACGAGGACGAGAACCAGAGCCCCAGGAGCTTCCAGAAGAAGACCAGGCACTACTTCATTGCTGC
TGTGGAGAGGCTGTGGGACTATGGCATGAGCAGCAGCCCCCATGTGCTGAGGAACAGGGCCCAGTCTG
GCTCTGTGCCCCAGTTCAAGAAGGTGGTGTTCCAGGAGTTCACTGATGGCAGCTTCACCCAGCCCCTG
TACAGAGGGGAGCTGAATGAGCACCTGGGCCTGCTGGGCCCCTACATCAGGGCTGAGGTGGAGGACAA
CATCATGGTGACCTTCAGGAACCAGGCCAGCAGGCCCTACAGCTTCTACAGCAGCCTGATCAGCTATG
AGGAGGACCAGAGGCAGGGGGCTGAGCCCAGGAAGAACTTTGTGAAGCCCAATGAAACCAAGACCTAC
TTCTGGAAGGTGCAGCACCACATGGCCCCCACCAAGGATGAGTTTGACTGCAAGGCCTGGGCCTACTT
CTCTGATGTGGACCTGGAGAAGGATGTGCACTCTGGCCTGATTGGCCCCCTGCTGGTGTGCCACACCA
ACACCCTGAACCCTGCCCATGGCAGGCAGGTGACTGTGCAGGAGTTTGCCCTGTTCTTCACCATCTTT
GATGAAACCAAGAGCTGGTACTTCACTGAGAACATGGAGAGGAACTGCAGGGCCCCCTGCAACATCCA
GATGGAGGACCCCACCTTCAAGGAGAACTACAGGTTCCATGCCATCAATGGCTACATCATGGACACCC
TGCCTGGCCTGGTGATGGCCCAGGACCAGAGGATCAGGTGGTACCTGCTGAGCATGGGCAGCAATGAG
AACATCCACAGCATCCACTTCTCTGGCCATGTGTTCACTGTGAGGAAGAAGGAGGAGTACAAGATGGC
CCTGTACAACCTGTACCCTGGGGTGTTTGAGACTGTGGAGATGCTGCCCAGCAAGGCTGGCATCTGGA
GGGTGGAGTGCCTGATTGGGGAGCACCTGCATGCTGGCATGAGCACCCTGTTCCTGGTGTACAGCAAC
AAGTGCCAGACCCCCCTGGGCATGGCCTCTGGCCACATCAGGGACTTCCAGATCACTGCCTCTGGCCA
GTATGGCCAGTGGGCCCCCAAGCTGGCCAGGCTGCACTACTCTGGCAGCATCAATGCCTGGAGCACCA
AGGAGCCCTTCAGCTGGATCAAGGTGGACCTGCTGGCCCCCATGATCATCCATGGCATCAAGACCCAG
GGGGCCAGGCAGAAGTTCAGCAGCCTGTACATCAGCCAGTTCATCATCATGTACAGCCTGGATGGCAA
GAAGTGGCAGACCTACAGGGGCAACAGCACTGGCACCCTGATGGTGTTCTTTGGCAATGTGGACAGCT
CTGGCATCAAGCACAACATCTTCAACCCCCCCATCATTGCCAGATACATCAGGCTGCACCCCACCCAC
TACAGCATCAGGAGCACCCTGAGGATGGAGCTGATGGGCTGTGACCTGAACAGCTGCAGCATGCCCCT
GGGCATGGAGAGCAAGGCCATCTCTGATGCCCAGATCACTGCCAGCAGCTACTTCACCAACATGTTTG
CCACCTGGAGCCCCAGCAAGGCCAGGCTGCACCTGCAGGGCAGGAGCAATGCCTGGAGGCCCCAGGTC
AACAACCCCAAGGAGTGGCTGCAGGTGGACTTCCAGAAGACCATGAAGGTGACTGGGGTGACCACCCA
GGGGGTGAAGAGCCTGCTGACCAGCATGTATGTGAAGGAGTTCCTGATCAGCAGCAGCCAGGATGGCC
ACCAGTGGACCCTGTTCTTCCAGAATGGCAAGGTGAAGGTGTTCCAGGGCAACCAGGACAGCTTCACC
CCTGTGGTGAACAGCCTGGACCCCCCCCTGCTGACCAGATACCTGAGGATTCACCCCCAGAGCTGGGT
GCACCAGATTGCCCTGAGGATGGAGGTGCTGGGCTGTGAGGCCCAGGACCTGTACTGATTAATTAAGA
GCATCTTACCGCCATTTATTCCCATATTTGTTCTGTTTTTCTTGATTTGGGTATACATTTAAATGTTA
ATAAAACAAAATGGTGGGGCAATCATTTACATTTTTAGGGATATGTAATTACTAGTTCAGGTGTATTG
CCACAAGACAAACATGTTAAGAAACTTTCCCGTTATTTACGCTCTGTTCCTGTTAATCAACCTCTGGA
TTACAAAATTTGTGAAAGATTGACTGATATTCTTAACTATGTTGCTCCTTTTACGCTGTGTGGATATG
CTGCTTTATAGCCTCTGTATCTAGCTATTGCTTCCCGTACGGCTTTCGTTTTCTCCTCCTTGTATAAA
TCCTGGTTGCTGTCTCTTTTAGAGGAGTTGTGGCCCGTTGTCCGTCAACGTGGCGTGGTGTGCTCTGT
GTTTGCTGACGCAACCCCCACTGGCTGGGGCATTGCCACCACCTGTCAACTCCTTTCTGGGACTTTCG
CTTTCCCCCTCCCGATCGCCACGGCAGAACTCATCGCCGCCTGCCTTGCCCGCTGCTGGACAGGGGCT
AGGTTGCTGGGCACTGATAATTCCGTGGTGTTGTCTGTGCCTTCTAGTTGCCAGCCATCTGTTGTTTG
CCCCTCCCCCGTGCCTTCCTTGACCCTGGAAGGTGCCACTCCCACTGTCCTTTCCTAATAAAATGAGG
AAATTGCATCGCATTGTCTGAGTAGGTGTCATTCTATTCTGGGGGGTGGGGTGGGGCAGGACAGCAAG
GGGGAGGATTGGGAAGACAATAGCAGGCATGCTGGGGATGCGGTGGGCTCTATGGCTCTAGAGCATGG
CTACGTAGATAAGTAGCATGGCGGGTTAATCATTAACTACACCTGCAGGAGGAACCCCTAGTGATGGA
GTTGGCCACTCCCTCTCTGCGCGCTCGCTCGCTCACTGAGGCCGGGCGACCAAAGGTCGCCCGACGCC
CGGGCTTTGCCCGGGCGGCCTCAGTG
041 (Lane 5; 6242 bp; hFVIII ORF 985-5376)
GAGGCCGCCCGGGCAAAGCCCGGGCGTCGGGCGACCTTTGGTCGCCCGGCCTCAGTGAGCGAGCGAGC
GCGCAGAGAGGGAGTGGCCAACTCCATCACTAGGGGTTCCTCCTGCAGGTGTAGTTAATGATTAACCC
GCCATGCTACTTATCTACGTAGCCATGCTCTAGAGCCATAGAGCCCACCGCATCCCCAGCATGCCTGC
TATTGTCTTCCCAATCCTCCCCCTTGCTGTCCTGCCCCACCCCACCCCCCAGAATAGAATGACACCTA
CTCAGACAATGCGATGCAATTTCCTCATTTTATTAGGAAAGGACAGTGGGAGTGGCACCTTCCAGGGT
CAAGGAAGGCACGGGGGAGGGGCAAACAACAGATGGCTGGCAACTAGAAGGCACAGACAACACCACGG
AATTATCAGTGCCCAGCAACCTAGCCCCTGTCCAGCAGCGGGCAAGGCAGGCGGCGATGAGTTCTGCC
GTGGCGATCGGGAGGGGGAAAGCGAAAGTCCCAGAAAGGAGTTGACAGGTGGTGGCAATGCCCCAGCC
AGTGGGGGTTGCGTCAGCAAACACAGAGCACACCACGCCACGTTGACGGACAACGGGCCACAACTCCT
CTAAAAGAGACAGCAACCAGGATTTATACAAGGAGGAGAAAACGAAAGCCGTACGGGAAGCAATAGCT
AGATACAGAGGCTATAAAGCAGCATATCCACACAGCGTAAAAGGAGCAACATAGTTAAGAATATCAGT
CAATCTTTCACAAATTTTGTAATCCAGAGGTTGATTAACAGGAACAGAGCGTAAATAACGGGAAAGTT
TCTTAACATGTTTGTCTTGTGGCAATACACCTGAACTAGTAATTACATATCCCTAAAAATGTAAATGA
TTGCCCCACCATTTTGTTTTATTAACATTTAAATGTATACCCAAATCAAGAAAAACAGAACAAATATG
GGAATAAATGGCGGTAAGATGCTCTTAATTAATCAGTACAGGTCCTGGGCCTCACAGCCCAGCACCTC
CATCCTCAGGGCAATCTGGTGCACCCAGCTCTGGGGGTGAATCCTCAGGTATCTGGTCAGCAGGGGGG
GGTCCAGGCTGTTCACCACAGGGGTGAAGCTGTCCTGGTTGCCCTGGAACACCTTCACCTTGCCATTC
TGGAAGAACAGGGTCCACTGGTGGCCATCCTGGCTGCTGCTGATCAGGAACTCCTTCACATACATGCT
GGTCAGCAGGCTCTTCACCCCCTGGGTGGTCACCCCAGTCACCTTCATGGTCTTCTGGAAGTCCACCT
GCAGCCACTCCTTGGGGTTGTTGACCTGGGGCCTCCAGGCATTGCTCCTGCCCTGCAGGTGCAGCCTG
GCCTTGCTGGGGCTCCAGGTGGCAAACATGTTGGTGAAGTAGCTGCTGGCAGTGATCTGGGCATCAGA
GATGGCCTTGCTCTCCATGCCCAGGGGCATGCTGCAGCTGTTCAGGTCACAGCCCATCAGCTCCATCC
TCAGGGTGCTCCTGATGCTGTAGTGGGTGGGGTGCAGCCTGATGTATCTGGCAATGATGGGGGGGTTG
AAGATGTTGTGCTTGATGCCAGAGCTGTCCACATTGCCAAAGAACACCATCAGGGTGCCAGTGCTGTT
GCCCCTGTAGGTCTGCCACTTCTTGCCATCCAGGCTGTACATGATGATGAACTGGCTGATGTACAGGC
TGCTGAACTTCTGCCTGGCCCCCTGGGTCTTGATGCCATGGATGATCATGGGGGCCAGCAGGTCCACC
TTGATCCAGCTGAAGGGCTCCTTGGTGCTCCAGGCATTGATGCTGCCAGAGTAGTGCAGCCTGGCCAG
CTTGGGGGCCCACTGGCCATACTGGCCAGAGGCAGTGATCTGGAAGTCCCTGATGTGGCCAGAGGCCA
TGCCCAGGGGGGTCTGGCACTTGTTGCTGTACACCAGGAACAGGGTGCTCATGCCAGCATGCAGGTGC
TCCCCAATCAGGCACTCCACCCTCCAGATGCCAGCCTTGCTGGGCAGCATCTCCACAGTCTCAAACAC
CCCAGGGTACAGGTTGTACAGGGCCATCTTGTACTCCTCCTTCTTCCTCACAGTGAACACATGGCCAG
AGAAGTGGATGCTGTGGATGTTCTCATTGCTGCCCATGCTCAGCAGGTACCACCTGATCCTCTGGTCC
TGGGCCATCACCAGGCCAGGCAGGGTGTCCATGATGTAGCCATTGATGGCATGGAACCTGTAGTTCTC
CTTGAAGGTGGGGTCCTCCATCTGGATGTTGCAGGGGGCCCTGCAGTTCCTCTCCATGTTCTCAGTGA
AGTACCAGCTCTTGGTTTCATCAAAGATGGTGAAGAACAGGGCAAACTCCTGCACAGTCACCTGCCTG
CCATGGGCAGGGTTCAGGGTGTTGGTGTGGCACACCAGCAGGGGGCCAATCAGGCCAGAGTGCACATC
CTTCTCCAGGTCCACATCAGAGAAGTAGGCCCAGGCCTTGCAGTCAAACTCATCCTTGGTGGGGGCCA
TGTGGTGCTGCACCTTCCAGAAGTAGGTCTTGGTTTCATTGGGCTTCACAAAGTTCTTCCTGGGCTCA
GCCCCCTGCCTCTGGTCCTCCTCATAGCTGATCAGGCTGCTGTAGAAGCTGTAGGGCCTGCTGGCCTG
GTTCCTGAAGGTCACCATGATGTTGTCCTCCACCTCAGCCCTGATGTAGGGGCCCAGCAGGCCCAGGT
GCTCATTCAGCTCCCCTCTGTACAGGGGCTGGGTGAAGCTGCCATCAGTGAACTCCTGGAACACCACC
TTCTTGAACTGGGGCACAGAGCCAGACTGGGCCCTGTTCCTCAGCACATGGGGGCTGCTGCTCATGCC
ATAGTCCCACAGCCTCTCCACAGCAGCAATGAAGTAGTGCCTGGTCTTCTTCTGGAAGCTCCTGGGGC
TCTGGTTCTCGTCCTCGTCGTAGATGTCAAAGTCCTCCTTCTTCATCTCCACAGAGATGGTGTCATCA
TAGTCAATCTCCTCCTGGTCAGACTGCAGGGTGGTATTCTCTGGGATGGTGGTGGCATTGAACTGCTT
CTGCCTGGTGCTGGGGTGCCTGCTATTCTGGCTGAAGCTCCTGGGCTCAATGGCATTGTTCTTGCTCA
GCAGGTAGGCAGAGATGTCCTCATAGCTGTCCTCATAGTAGTCCCCAGTGTTCTTGTCACAGCTGGAG
ACTTTCAGCAGGGCAGTCATGCCCCTGTTCCTGAAGTCAGAGTTGTGGCAGCCCAGAATCCACAGGCC
AGGGTTCTCCATGCTCATGAACACAGTCTCCCCAGAGAAGGGGAACAGGGTCAGGGTGTCCTCATACA
CCATCTTGTGCTTGAAGGTGTAGCCAGAGAAGAACACAGACAGGAAGTCAGTCTGGGCCCCAATGCTC
AGGATGTACCAGTAGGCCACCTCATGCAGGCACACAGACAGCTGCAGGCTGTCAAACACATAGCCATT
GATGCTGTGCATGATGTTGCTGGCCTGGAACTCAGGGTCCTCCAGCTGCACCCCAGCAGGGTTGGGCA
GGAACCTCTGGATGTTCTCAGTCAGGTACCAGCTCCTGTTCTCATCAAACACAGAGAACAGGATCACA
TTCCTCTTGTCAGACATGATCTGGTTGCCCCTCTGGTCCACAGACTCCTTGTAGCAGATCAGCAGGGG
GCCAATCAGGCCAGAGGCCAGGTCCCTCTCCATGTTCACAAAGCTGCTGTAGTATCTGGTCAGGCACC
TGGGGTCAGACTTGGTGGGGCCATCCTCCACAGTCACAGTCCACTTGTACTTGAAGATCTCCCCAGGC
AGGATGGGGAAGTCCTTCAGGTGCTTCACCCCCTTGGGCAGCCTCCTGCTGTACAGGGGCCTCACATC
AGTGATGCCATGGGGGTAGATGTTGTAGGGCCTGCTGGCCTGGTTCTTGAAGATGATCAGCAGGGTGT
CCCCCACCTCCCCATACAGCAGGGGGCCCAGGATGCCAGACTCATGCTGGATGGCCTCCCTGGTCTTG
AAGGTTTCATCAGTGTAGGCCATGAACCTGACCTTCTTGTACTTCCTGCCAATCCTCTGGGGGCCATT
GTTCAGGTACTGGCTCTTGTAGCTCCTGTCATCAGGGGCCAGCACCAGGGGGGCATAGTCCCAGTCCT
CCTCCTCAGCAGCAATGTAGTGCACCCAGGTCTTGGGGTGCTTCTTGGCCACAGACCTGATCTGGATG
AAGCTGGGGCTGTTGTCATCATCAAACCTCACCACATCCATCTCAGAGTCAGTCAGGTCATCATCATA
GTCCTCAGCCTCCTCATTGTTCTTCATCCTCAGCTGGGGCTCCTCAGGGCAGCTGTCCACCTTCACAT
AGGCCTCCATGCCATCATGCTGGTGGCTGCTGATGTGGCAGAACAGCAGGAACTGGCCCAGGTCCATC
AGCAGGGTCTGGGCAGTCAGGAAGGTGATGGGGCTGATCTCCAGGCTGGCCTGCCTGTGGTTCCTGAC
CAGGAAGGTGTGGCCCTCCAGGAAGATGCTGTGCACCTCAGGGGTGGTGCCCATGCCAATCACATGCC
AGTACACAGACTTCCTGTGGCAGCCAATCAGGCCAGGCAGGCTCCTGTTCACATAGCCATTCACAGTG
TGCATCTTGGGCCAGGCCCTGGCAGAGGCAGCATCCCTGTCCTGCATCAGGCTGTTCTTGGTTTCAGA
GTGCCAGCTCTTGCCCTCATCAAACACAGCAAACAGCAGGATGAACTTGTGCAGGGTCTGGGTCTTCT
CCTTGGCCAGGCTGCCCTCCCTGCACACCAGCAGGGCCCCAATCAGGCCAGAGTTCAGGTCCTTCACC
AGGTCCACATGGCTCAGGTAGCTGTAGGTCAGGCACAGGGGGTCAGAGGCCATGGGGCCATTCTCCTT
CAGCACCTGCCACACATAGGTGTGGCTGCCCCCAGGGAACACCTTGTCATCCTCCTTCTCCCTCTGGC
TGGTCTGGTCATCATACTCAGCCCCCTCAGAGGCCTTCCAGTAGCTCACCCCCACAGCATGCAGGCTC
ACAGGGTGGCTGGCCATGTTCTTCAGGGTGATCACCACAGTGTCATACACCTCAGCCTGGATGGTGGG
GCCCAGCAGGCCCATCCAGGGGGGCCTGGGCTTGGCAATGTTGAACAGGTGGTCAGTGAACTCCACAA
ACAGGGTCTTCTTGTACACCACAGAGGTGTTGAAGGGGAAGCTCTTGGGCACTCTGGGGGGGAACCTG
GCATCCACAGGCAGCTCCCCCAGGTCAGACTGCATGTAGTCCCAGCTCAGCTCCACAGCCCCCAGGTA
GTATCTCCTGGTGGCAGAGAAGCAGAACCTCAGCAGGCACAGGAAGAAGCAGGTGCTCAGCTCAATCT
GCATGGTGGCGGCGTTTAAACCAACCTGAAAAAAAGTGATTTCAGGCAGGTGCTCCAGGTAATTAAAC
ATTAATACCCCACCAACCAACCATCCCTTAAACCCTTACCTCTTCAGGAGCTTGTGGATCTGTGTGAC
GGCTTCTCCTGGTGAAGGGGCTTTTATACCCCCTCCTTCCAACCCAGGCTGCTGATCCCTGCCAAGCT
GACTCCAAACCTGCTGATTCTGATTATTGACTTAGTCAACAAAAGGAGAATAAGTAACCTACACAAAT
ATGAACCTTGCCTAGGGAGATTAGAGTATCGGAACACTCGCTCTACGAAATGTGCAGACAGACGGGGA
TCCTCTAGAGCTACCTGCTGATCGCCCGGCCCCTGTTCAAACATGTCCTAATACTCTGTCTCTGCAAG
GGTCATCAGTAGTTTTCCATCTTACTCAACATCCTCCCAGTGGGTACCGTGGACTTAGCCCCTGTTTG
CTCCTCCGATAACTGGGGTGACCTTGGTTAATATTCACCAGCAGCCTCCCCCGGTGGACTTAGCCCCT
GTTTGCTCCTCCGATAACTGGGGTGACCTTGGTTAATATTCACCAGCAGCCTCCCCCGGTGGACTTAG
CCCCTGTTTGCTCCTCCGATAACTGGGGTGACCTTGGTTAATATTCACCAGCAGCCTCCCCCGCGGCC
GCGATAAGTAGCATGGCGGGTTAATCATTAACTACAAGGAACCCCTAGTGATGGAGCCTCAGCTTGGC
CACTCCCTCTCTGCGCGCTCGCTCGCTCACTGAGGCCGCCCGGGCAAAGCCCGGGCGTCGGGCGACCT
TTGGTCGCCCGGCCTCAGTGAGCGAGCGAGCGCGCAGAGAGGGAGTGGCCAAGC
656 (Lanes 9-10; 6214 bp; hFVIII ORF 835-5226)
CTCACTGAGGCCGCCCGGGCAAAGCCCGGGCGTCGGGCGACCTTTGGTCGCCCGNCCTCAGTGAGCGA
GCGAGCGCGCAGAGAGGGAGTGGCCAAGCTGAGGCTCCATCACTAGGGGTTCCTTGTAGTTAATGATT
AACCCGCCATGCTACTTATCGCGGCCGCGGGGGAGGCTGCTGGTGAATATTAACCAAGGTCACCCCAG
TTATCGGAGGAGCAAACAGGGGCTAAGTCCACCGGGGGAGGCTGCTGGTGAATATTAACCAAGGTCAC
CCCAGTTATCGGAGGAGCAAACAGGGGCTAAGTCCACCGGGGGAGGCTGCTGGTGAATATTAACCAAG
GTCACCCCAGTTATCGGAGGAGCAAACAGGGGCTAAGTCCACGGTACCCACTGGGAGGATGTTGAGTA
AGATGGAAAACTACTGATGACCCTTGCAGAGACAGAGTATTAGGACATGTTTGAACAGGGGCCGGGCG
ATCAGCAGGTAGCTCTAGAGGATCCCCGTCTGTCTGCACATTTCGTAGAGCGAGTGTTCCGATACTCT
AATCTCCCTAGGCAAGGTTCATATTTGTGTAGGTTACTTATTCTCCTTTTGTTGACTAAGTCAATAAT
CAGAATCAGCAGGTTTGGAGTCAGCTTGGCAGGGATCAGCAGCCTGGGTTGGAAGGAGGGGGTATAAA
AGCCCCTTCACCAGGAGAAGCCGTCACACAGATCCACAAGCTCCTGAAGAGGTAAGGGTTTAAGGGAT
GGTTGGTTGGTGGGGTATTAATGTTTAATTACCTGGAGCACCTGCCTGAAATCACTTTTTTTCAGGTT
GGTTTAAACGCCGCCACCATGCAGATTGAGCTGAGCACCTGCTTCTTCCTGTGCCTGCTGAGGTTCTG
CTTCTCTGCCACCAGGAGATACTACCTGGGGGCTGTGGAGCTGAGCTGGGACTACATGCAGTCTGACC
TGGGGGAGCTGCCTGTGGATGCCAGGTTCCCCCCCAGAGTGCCCAAGAGCTTCCCCTTCAACACCTCT
GTGGTGTACAAGAAGACCCTGTTTGTGGAGTTCACTGACCACCTGTTCAACATTGCCAAGCCCAGGCC
CCCCTGGATGGGCCTGCTGGGCCCCACCATCCAGGCTGAGGTGTATGACACTGTGGTGATCACCCTGA
AGAACATGGCCAGCCACCCTGTGAGCCTGCATGCTGTGGGGGTGAGCTACTGGAAGGCCTCTGAGGGG
GCTGAGTATGATGACCAGACCAGCCAGAGGGAGAAGGAGGATGACAAGGTGTTCCCTGGGGGCAGCCA
CACCTATGTGTGGCAGGTGCTGAAGGAGAATGGCCCCATGGCCTCTGACCCCCTGTGCCTGACCTACA
GCTACCTGAGCCATGTGGACCTGGTGAAGGACCTGAACTCTGGCCTGATTGGGGCCCTGCTGGTGTGC
AGGGAGGGCAGCCTGGCCAAGGAGAAGACCCAGACCCTGCACAAGTTCATCCTGCTGTTTGCTGTGTT
TGATGAGGGCAAGAGCTGGCACTCTGAAACCAAGAACAGCCTGATGCAGGACAGGGATGCTGCCTCTG
CCAGGGCCTGGCCCAAGATGCACACTGTGAATGGCTATGTGAACAGGAGCCTGCCTGGCCTGATTGGC
TGCCACAGGAAGTCTGTGTACTGGCATGTGATTGGCATGGGCACCACCCCTGAGGTGCACAGCATCTT
CCTGGAGGGCCACACCTTCCTGGTCAGGAACCACAGGCAGGCCAGCCTGGAGATCAGCCCCATCACCT
TCCTGACTGCCCAGACCCTGCTGATGGACCTGGGCCAGTTCCTGCTGTTCTGCCACATCAGCAGCCAC
CAGCATGATGGCATGGAGGCCTATGTGAAGGTGGACAGCTGCCCTGAGGAGCCCCAGCTGAGGATGAA
GAACAATGAGGAGGCTGAGGACTATGATGATGACCTGACTGACTCTGAGATGGATGTGGTGAGGTTTG
ATGATGACAACAGCCCCAGCTTCATCCAGATCAGGTCTGTGGCCAAGAAGCACCCCAAGACCTGGGTG
CACTACATTGCTGCTGAGGAGGAGGACTGGGACTATGCCCCCCTGGTGCTGGCCCCTGATGACAGGAG
CTACAAGAGCCAGTACCTGAACAATGGCCCCCAGAGGATTGGCAGGAAGTACAAGAAGGTCAGGTTCA
TGGCCTACACTGATGAAACCTTCAAGACCAGGGAGGCCATCCAGCATGAGTCTGGCATCCTGGGCCCC
CTGCTGTATGGGGAGGTGGGGGACACCCTGCTGATCATCTTCAAGAACCAGGCCAGCAGGCCCTACAA
CATCTACCCCCATGGCATCACTGATGTGAGGCCCCTGTACAGCAGGAGGCTGCCCAAGGGGGTGAAGC
ACCTGAAGGACTTCCCCATCCTGCCTGGGGAGATCTTCAAGTACAAGTGGACTGTGACTGTGGAGGAT
GGCCCCACCAAGTCTGACCCCAGGTGCCTGACCAGATACTACAGCAGCTTTGTGAACATGGAGAGGGA
CCTGGCCTCTGGCCTGATTGGCCCCCTGCTGATCTGCTACAAGGAGTCTGTGGACCAGAGGGGCAACC
AGATCATGTCTGACAAGAGGAATGTGATCCTGTTCTCTGTGTTTGATGAGAACAGGAGCTGGTACCTG
ACTGAGAACATCCAGAGGTTCCTGCCCAACCCTGCTGGGGTGCAGCTGGAGGACCCTGAGTTCCAGGC
CAGCAACATCATGCACAGCATCAATGGCTATGTGTTTGACAGCCTGCAGCTGTCTGTGTGCCTGCATG
AGGTGGCCTACTGGTACATCCTGAGCATTGGGGCCCAGACTGACTTCCTGTCTGTGTTCTTCTCTGGC
TACACCTTCAAGCACAAGATGGTGTATGAGGACACCCTGACCCTGTTCCCCTTCTCTGGGGAGACTGT
GTTCATGAGCATGGAGAACCCTGGCCTGTGGATTCTGGGCTGCCACAACTCTGACTTCAGGAACAGGG
GCATGACTGCCCTGCTGAAAGTCTCCAGCTGTGACAAGAACACTGGGGACTACTATGAGGACAGCTAT
GAGGACATCTCTGCCTACCTGCTGAGCAAGAACAATGCCATTGAGCCCAGGAGCTTCAGCCAGAATAG
CAGGCACCCCAGCACCAGGCAGAAGCAGTTCAATGCCACCACCATCCCAGAGAATACCACCCTGCAGT
CTGACCAGGAGGAGATTGACTATGATGACACCATCTCTGTGGAGATGAAGAAGGAGGACTTTGACATC
TACGACGAGGACGAGAACCAGAGCCCCAGGAGCTTCCAGAAGAAGACCAGGCACTACTTCATTGCTGC
TGTGGAGAGGCTGTGGGACTATGGCATGAGCAGCAGCCCCCATGTGCTGAGGAACAGGGCCCAGTCTG
GCTCTGTGCCCCAGTTCAAGAAGGTGGTGTTCCAGGAGTTCACTGATGGCAGCTTCACCCAGCCCCTG
TACAGAGGGGAGCTGAATGAGCACCTGGGCCTGCTGGGCCCCTACATCAGGGCTGAGGTGGAGGACAA
CATCATGGTGACCTTCAGGAACCAGGCCAGCAGGCCCTACAGCTTCTACAGCAGCCTGATCAGCTATG
AGGAGGACCAGAGGCAGGGGGCTGAGCCCAGGAAGAACTTTGTGAAGCCCAATGAAACCAAGACCTAC
TTCTGGAAGGTGCAGCACCACATGGCCCCCACCAAGGATGAGTTTGACTGCAAGGCCTGGGCCTACTT
CTCTGATGTGGACCTGGAGAAGGATGTGCACTCTGGCCTGATTGGCCCCCTGCTGGTGTGCCACACCA
ACACCCTGAACCCTGCCCATGGCAGGCAGGTGACTGTGCAGGAGTTTGCCCTGTTCTTCACCATCTTT
GATGAAACCAAGAGCTGGTACTTCACTGAGAACATGGAGAGGAACTGCAGGGCCCCCTGCAACATCCA
GATGGAGGACCCCACCTTCAAGGAGAACTACAGGTTCCATGCCATCAATGGCTACATCATGGACACCC
TGCCTGGCCTGGTGATGGCCCAGGACCAGAGGATCAGGTGGTACCTGCTGAGCATGGGCAGCAATGAG
AACATCCACAGCATCCACTTCTCTGGCCATGTGTTCACTGTGAGGAAGAAGGAGGAGTACAAGATGGC
CCTGTACAACCTGTACCCTGGGGTGTTTGAGACTGTGGAGATGCTGCCCAGCAAGGCTGGCATCTGGA
GGGTGGAGTGCCTGATTGGGGAGCACCTGCATGCTGGCATGAGCACCCTGTTCCTGGTGTACAGCAAC
AAGTGCCAGACCCCCCTGGGCATGGCCTCTGGCCACATCAGGGACTTCCAGATCACTGCCTCTGGCCA
GTATGGCCAGTGGGCCCCCAAGCTGGCCAGGCTGCACTACTCTGGCAGCATCAATGCCTGGAGCACCA
AGGAGCCCTTCAGCTGGATCAAGGTGGACCTGCTGGCCCCCATGATCATCCATGGCATCAAGACCCAG
GGGGCCAGGCAGAAGTTCAGCAGCCTGTACATCAGCCAGTTCATCATCATGTACAGCCTGGATGGCAA
GAAGTGGCAGACCTACAGGGGCAACAGCACTGGCACCCTGATGGTGTTCTTTGGCAATGTGGACAGCT
CTGGCATCAAGCACAACATCTTCAACCCCCCCATCATTGCCAGATACATCAGGCTGCACCCCACCCAC
TACAGCATCAGGAGCACCCTGAGGATGGAGCTGATGGGCTGTGACCTGAACAGCTGCAGCATGCCCCT
GGGCATGGAGAGCAAGGCCATCTCTGATGCCCAGATCACTGCCAGCAGCTACTTCACCAACATGTTTG
CCACCTGGAGCCCCAGCAAGGCCAGGCTGCACCTGCAGGGCAGGAGCAATGCCTGGAGGCCCCAGGTC
AACAACCCCAAGGAGTGGCTGCAGGTGGACTTCCAGAAGACCATGAAGGTGACTGGGGTGACCACCCA
GGGGGTGAAGAGCCTGCTGACCAGCATGTATGTGAAGGAGTTCCTGATCAGCAGCAGCCAGGATGGCC
ACCAGTGGACCCTGTTCTTCCAGAATGGCAAGGTGAAGGTGTTCCAGGGCAACCAGGACAGCTTCACC
CCTGTGGTGAACAGCCTGGACCCCCCCCTGCTGACCAGATACCTGAGGATTCACCCCCAGAGCTGGGT
GCACCAGATTGCCCTGAGGATGGAGGTGCTGGGCTGTGAGGCCCAGGACCTGTACTGATTAATTAAGA
GCATCTTACCGCCATTTATTCCCATATTTGTTCTGTTTTTCTTGATTTGGGTATACATTTAAATGTTA
ATAAAACAAAATGGTGGGGCAATCATTTACATTTTTAGGGATATGTAATTACTAGTTCAGGTGTATTG
CCACAAGACAAACATGTTAAGAAACTTTCCCGTTATTTACGCTCTGTTCCTGTTAATCAACCTCTGGA
TTACAAAATTTGTGAAAGATTGACTGATATTCTTAACTATGTTGCTCCTTTTACGCTGTGTGGATATG
CTGCTTTATAGCCTCTGTATCTAGCTATTGCTTCCCGTACGGCTTTCGTTTTCTCCTCCTTGTATAAA
TCCTGGTTGCTGTCTCTTTTAGAGGAGTTGTGGCCCGTTGTCCGTCAACGTGGCGTGGTGTGCTCTGT
GTTTGCTGACGCAACCCCCACTGGCTGGGGCATTGCCACCACCTGTCAACTCCTTTCTGGGACTTTCG
CTTTCCCCCTCCCGATCGCCACGGCAGAACTCATCGCCGCCTGCCTTGCCCGCTGCTGGACAGGGGCT
AGGTTGCTGGGCACTGATAATTCCGTGGTGTTGTCTGTGCCTTCTAGTTGCCAGCCATCTGTTGTTTG
CCCCTCCCCCGTGCCTTCCTTGACCCTGGAAGGTGCCACTCCCACTGTCCTTTCCTAATAAAATGAGG
AAATTGCATCGCATTGTCTGAGTAGGTGTCATTCTATTCTGGGGGGTGGGGTGGGGCAGGACAGCAAG
GGGGAGGATTGGGAAGACAATAGCAGGCATGCTGGGGATGCGGTGGGCTCTATGGCTCTAGAGCATGG
CTACGTAGATAAGTAGCATGGCGGGTTAATCATTAACTACACCTGCAGGAGGAACCCCTAGTGATGGA
GTTGGCCACTCCCTCTCTGCGCGCTCGCTCGCTCACTGAGGCCGGGCGACCAAAGGTCGCCCGACGCC
CGGGCTTTGCCCGGGCGGCCTCAGTG
042 (Lane 10; 6201 bp; hFVIII ORF 985-5376)
GAGGCCGCCCGGGCAAAGCCCGGGCGTCGGGCGACCTTTGGTCGCCCGGCCTCAGTGAGCGAGCGAGC
GCGCAGAGAGGGAGTGGCCAACTCCATCACTAGGGGTTCCTCCTGCAGGTGTAGTTAATGATTAACCC
GCCATGCTACTTATCTACGTAGCCATGCTCTAGAGCCATAGAGCCCACCGCATCCCCAGCATGCCTGC
TATTGTCTTCCCAATCCTCCCCCTTGCTGTCCTGCCCCACCCCACCCCCCAGAATAGAATGACACCTA
CTCAGACAATGCGATGCAATTTCCTCATTTTATTAGGAAAGGACAGTGGGAGTGGCACCTTCCAGGGT
CAAGGAAGGCACGGGGGAGGGGCAAACAACAGATGGCTGGCAACTAGAAGGCACAGACAACACCACGG
AATTATCAGTGCCCAGCAACCTAGCCCCTGTCCAGCAGCGGGCAAGGCAGGCGGCGATGAGTTCTGCC
GTGGCGATCGGGAGGGGGAAAGCGAAAGTCCCAGAAAGGAGTTGACAGGTGGTGGCAATGCCCCAGCC
AGTGGGGGTTGCGTCAGCAAACACAGAGCACACCACGCCACGTTGACGGACAACGGGCCACAACTCCT
CTAAAAGAGACAGCAACCAGGATTTATACAAGGAGGAGAAAACGAAAGCCGTACGGGAAGCAATAGCT
AGATACAGAGGCTATAAAGCAGCATATCCACACAGCGTAAAAGGAGCAACATAGTTAAGAATATCAGT
CAATCTTTCACAAATTTTGTAATCCAGAGGTTGATTAACAGGAACAGAGCGTAAATAACGGGAAAGTT
TCTTAACATGTTTGTCTTGTGGCAATACACCTGAACTAGTAATTACATATCCCTAAAAATGTAAATGA
TTGCCCCACCATTTTGTTTTATTAACATTTAAATGTATACCCAAATCAAGAAAAACAGAACAAATATG
GGAATAAATGGCGGTAAGATGCTCTTAATTAATCAGTACAGGTCCTGGGCCTCACAGCCCAGCACCTC
CATCCTCAGGGCAATCTGGTGCACCCAGCTCTGGGGGTGAATCCTCAGGTATCTGGTCAGCAGGGGGG
GGTCCAGGCTGTTCACCACAGGGGTGAAGCTGTCCTGGTTGCCCTGGAACACCTTCACCTTGCCATTC
TGGAAGAACAGGGTCCACTGGTGGCCATCCTGGCTGCTGCTGATCAGGAACTCCTTCACATACATGCT
GGTCAGCAGGCTCTTCACCCCCTGGGTGGTCACCCCAGTCACCTTCATGGTCTTCTGGAAGTCCACCT
GCAGCCACTCCTTGGGGTTGTTGACCTGGGGCCTCCAGGCATTGCTCCTGCCCTGCAGGTGCAGCCTG
GCCTTGCTGGGGCTCCAGGTGGCAAACATGTTGGTGAAGTAGCTGCTGGCAGTGATCTGGGCATCAGA
GATGGCCTTGCTCTCCATGCCCAGGGGCATGCTGCAGCTGTTCAGGTCACAGCCCATCAGCTCCATCC
TCAGGGTGCTCCTGATGCTGTAGTGGGTGGGGTGCAGCCTGATGTATCTGGCAATGATGGGGGGGTTG
AAGATGTTGTGCTTGATGCCAGAGCTGTCCACATTGCCAAAGAACACCATCAGGGTGCCAGTGCTGTT
GCCCCTGTAGGTCTGCCACTTCTTGCCATCCAGGCTGTACATGATGATGAACTGGCTGATGTACAGGC
TGCTGAACTTCTGCCTGGCCCCCTGGGTCTTGATGCCATGGATGATCATGGGGGCCAGCAGGTCCACC
TTGATCCAGCTGAAGGGCTCCTTGGTGCTCCAGGCATTGATGCTGCCAGAGTAGTGCAGCCTGGCCAG
CTTGGGGGCCCACTGGCCATACTGGCCAGAGGCAGTGATCTGGAAGTCCCTGATGTGGCCAGAGGCCA
TGCCCAGGGGGGTCTGGCACTTGTTGCTGTACACCAGGAACAGGGTGCTCATGCCAGCATGCAGGTGC
TCCCCAATCAGGCACTCCACCCTCCAGATGCCAGCCTTGCTGGGCAGCATCTCCACAGTCTCAAACAC
CCCAGGGTACAGGTTGTACAGGGCCATCTTGTACTCCTCCTTCTTCCTCACAGTGAACACATGGCCAG
AGAAGTGGATGCTGTGGATGTTCTCATTGCTGCCCATGCTCAGCAGGTACCACCTGATCCTCTGGTCC
TGGGCCATCACCAGGCCAGGCAGGGTGTCCATGATGTAGCCATTGATGGCATGGAACCTGTAGTTCTC
CTTGAAGGTGGGGTCCTCCATCTGGATGTTGCAGGGGGCCCTGCAGTTCCTCTCCATGTTCTCAGTGA
AGTACCAGCTCTTGGTTTCATCAAAGATGGTGAAGAACAGGGCAAACTCCTGCACAGTCACCTGCCTG
CCATGGGCAGGGTTCAGGGTGTTGGTGTGGCACACCAGCAGGGGGCCAATCAGGCCAGAGTGCACATC
CTTCTCCAGGTCCACATCAGAGAAGTAGGCCCAGGCCTTGCAGTCAAACTCATCCTTGGTGGGGGCCA
TGTGGTGCTGCACCTTCCAGAAGTAGGTCTTGGTTTCATTGGGCTTCACAAAGTTCTTCCTGGGCTCA
GCCCCCTGCCTCTGGTCCTCCTCATAGCTGATCAGGCTGCTGTAGAAGCTGTAGGGCCTGCTGGCCTG
GTTCCTGAAGGTCACCATGATGTTGTCCTCCACCTCAGCCCTGATGTAGGGGCCCAGCAGGCCCAGGT
GCTCATTCAGCTCCCCTCTGTACAGGGGCTGGGTGAAGCTGCCATCAGTGAACTCCTGGAACACCACC
TTCTTGAACTGGGGCACAGAGCCAGACTGGGCCCTGTTCCTCAGCACATGGGGGCTGCTGCTCATGCC
ATAGTCCCACAGCCTCTCCACAGCAGCAATGAAGTAGTGCCTGGTCTTCTTCTGGAAGCTCCTGGGGC
TCTGGTTCTCGTCCTCGTCGTAGATGTCAAAGTCCTCCTTCTTCATCTCCACAGAGATGGTGTCATCA
TAGTCAATCTCCTCCTGGTCAGACTGCAGGGTGGTATTCTCTGGGATGGTGGTGGCATTGAACTGCTT
CTGCCTGGTGCTGGGGTGCCTGCTATTCTGGCTGAAGCTCCTGGGCTCAATGGCATTGTTCTTGCTCA
GCAGGTAGGCAGAGATGTCCTCATAGCTGTCCTCATAGTAGTCCCCAGTGTTCTTGTCACAGCTGGAG
ACTTTCAGCAGGGCAGTCATGCCCCTGTTCCTGAAGTCAGAGTTGTGGCAGCCCAGAATCCACAGGCC
AGGGTTCTCCATGCTCATGAACACAGTCTCCCCAGAGAAGGGGAACAGGGTCAGGGTGTCCTCATACA
CCATCTTGTGCTTGAAGGTGTAGCCAGAGAAGAACACAGACAGGAAGTCAGTCTGGGCCCCAATGCTC
AGGATGTACCAGTAGGCCACCTCATGCAGGCACACAGACAGCTGCAGGCTGTCAAACACATAGCCATT
GATGCTGTGCATGATGTTGCTGGCCTGGAACTCAGGGTCCTCCAGCTGCACCCCAGCAGGGTTGGGCA
GGAACCTCTGGATGTTCTCAGTCAGGTACCAGCTCCTGTTCTCATCAAACACAGAGAACAGGATCACA
TTCCTCTTGTCAGACATGATCTGGTTGCCCCTCTGGTCCACAGACTCCTTGTAGCAGATCAGCAGGGG
GCCAATCAGGCCAGAGGCCAGGTCCCTCTCCATGTTCACAAAGCTGCTGTAGTATCTGGTCAGGCACC
TGGGGTCAGACTTGGTGGGGCCATCCTCCACAGTCACAGTCCACTTGTACTTGAAGATCTCCCCAGGC
AGGATGGGGAAGTCCTTCAGGTGCTTCACCCCCTTGGGCAGCCTCCTGCTGTACAGGGGCCTCACATC
AGTGATGCCATGGGGGTAGATGTTGTAGGGCCTGCTGGCCTGGTTCTTGAAGATGATCAGCAGGGTGT
CCCCCACCTCCCCATACAGCAGGGGGCCCAGGATGCCAGACTCATGCTGGATGGCCTCCCTGGTCTTG
AAGGTTTCATCAGTGTAGGCCATGAACCTGACCTTCTTGTACTTCCTGCCAATCCTCTGGGGGCCATT
GTTCAGGTACTGGCTCTTGTAGCTCCTGTCATCAGGGGCCAGCACCAGGGGGGCATAGTCCCAGTCCT
CCTCCTCAGCAGCAATGTAGTGCACCCAGGTCTTGGGGTGCTTCTTGGCCACAGACCTGATCTGGATG
AAGCTGGGGCTGTTGTCATCATCAAACCTCACCACATCCATCTCAGAGTCAGTCAGGTCATCATCATA
GTCCTCAGCCTCCTCATTGTTCTTCATCCTCAGCTGGGGCTCCTCAGGGCAGCTGTCCACCTTCACAT
AGGCCTCCATGCCATCATGCTGGTGGCTGCTGATGTGGCAGAACAGCAGGAACTGGCCCAGGTCCATC
AGCAGGGTCTGGGCAGTCAGGAAGGTGATGGGGCTGATCTCCAGGCTGGCCTGCCTGTGGTTCCTGAC
CAGGAAGGTGTGGCCCTCCAGGAAGATGCTGTGCACCTCAGGGGTGGTGCCCATGCCAATCACATGCC
AGTACACAGACTTCCTGTGGCAGCCAATCAGGCCAGGCAGGCTCCTGTTCACATAGCCATTCACAGTG
TGCATCTTGGGCCAGGCCCTGGCAGAGGCAGCATCCCTGTCCTGCATCAGGCTGTTCTTGGTTTCAGA
GTGCCAGCTCTTGCCCTCATCAAACACAGCAAACAGCAGGATGAACTTGTGCAGGGTCTGGGTCTTCT
CCTTGGCCAGGCTGCCCTCCCTGCACACCAGCAGGGCCCCAATCAGGCCAGAGTTCAGGTCCTTCACC
AGGTCCACATGGCTCAGGTAGCTGTAGGTCAGGCACAGGGGGTCAGAGGCCATGGGGCCATTCTCCTT
CAGCACCTGCCACACATAGGTGTGGCTGCCCCCAGGGAACACCTTGTCATCCTCCTTCTCCCTCTGGC
TGGTCTGGTCATCATACTCAGCCCCCTCAGAGGCCTTCCAGTAGCTCACCCCCACAGCATGCAGGCTC
ACAGGGTGGCTGGCCATGTTCTTCAGGGTGATCACCACAGTGTCATACACCTCAGCCTGGATGGTGGG
GCCCAGCAGGCCCATCCAGGGGGGCCTGGGCTTGGCAATGTTGAACAGGTGGTCAGTGAACTCCACAA
ACAGGGTCTTCTTGTACACCACAGAGGTGTTGAAGGGGAAGCTCTTGGGCACTCTGGGGGGGAACCTG
GCATCCACAGGCAGCTCCCCCAGGTCAGACTGCATGTAGTCCCAGCTCAGCTCCACAGCCCCCAGGTA
GTATCTCCTGGTGGCAGAGAAGCAGAACCTCAGCAGGCACAGGAAGAAGCAGGTGCTCAGCTCAATCT
GCATGGTGGCGGCGTTTAAACCAACCTGAAAAAAAGTGATTTCAGGCAGGTGCTCCAGGTAATTAAAC
ATTAATACCCCACCAACCAACCATCCCTTAAACCCTTACCTCTTCAGGAGCTTGTGGATCTGTGTGAC
GGCTTCTCCTGGTGAAGGGGCTTTTATACCCCCTCCTTCCAACCCAGGCTGCTGATCCCTGCCAAGCT
GACTCCAAACCTGCTGATTCTGATTATTGACTTAGTCAACAAAAGGAGAATAAGTAACCTACACAAAT
ATGAACCTTGCCTAGGGAGATTAGAGTATCGGAACACTCGCTCTACGAAATGTGCAGACAGACGGGGA
TCCTCTAGAGCTACCTGCTGATCGCCCGGCCCCTGTTCAAACATGTCCTAATACTCTGTCTCTGCAAG
GGTCATCAGTAGTTTTCCATCTTACTCAACATCCTCCCAGTGGGTACCGTGGACTTAGCCCCTGTTTG
CTCCTCCGATAACTGGGGTGACCTTGGTTAATATTCACCAGCAGCCTCCCCCGGTGGACTTAGCCCCT
GTTTGCTCCTCCGATAACTGGGGTGACCTTGGTTAATATTCACCAGCAGCCTCCCCCGGTGGACTTAG
CCCCTGTTTGCTCCTCCGATAACTGGGGTGACCTTGGTTAATATTCACCAGCAGCCTCCCCCGCGGCC
GCGATAAGTAGCATGGCGGGTTAATCATTAACTACAAGGAACCCCTAGTGATGGAGCCTCAGCTTGGC
CACTCCCTCTCTGCGCGCTCGCTCGCTCACTGAGGCCGCCCGGGCAAAGCCCGGGCGTCGGGCGACCT
TTGGTCGCCCGIC

### Example 3: An Alternate DNA Nicking Method Supported Rolling Circle Amplification (RCA)-Dependent Synthesis of Single-Stranded Closed-Ended DNA Vectors (SSD)

In this Example, single-stranded DNA (SSD) was produced from ceDNA manufactured using rolling circle amplification (RCA) of the plasmid template, as described in Example 2 above, with a difference that ceDNA nicking was performed with a distinct class of enzyme called Endonuclease V. Endonuclease V (EndoV) is a repair enzyme that specifically recognizes deoxyinosine, a deamination product of deoxyadenosine in DNA. Upon recognition of deoxyinosine, Endo V catalyzes the cleavage of the second DNA phosphodiester backbone 3′ to deoxyinosine, leaving a 3′-OH and 5′-phosphate. Importantly, deoxyinosine can be synthetically incorporated into the oligonucleotides allowing the recognition sequence to be encoded in ceDNA at the desired location to support nicking of the DNA backbone and SSD synthesis. An advantage of using EndoV is that the requirement for deoxyinosine means EndoV can be used regardless of the primary sequence of the transgene being encoded in the ceDNA precursor. In the case of Nb.BbvCI and other Type II restriction enzymes the transgene must not have recognition sites encoded in the transgene, which results in DNA nicks on both strands of the dsDNA. This is a design limitation that is solved by use of EndoV.

Briefly, 1.5 micrograms of purified ceDNA was treated with 0.75 units of Endonuclease V per microgram of DNA for 2 h at 37° C. in the presence of 1× NEBuffer 4 (50 mM Potassium acetate, 20 mM Tris-Acetate, 10 mM Magnesium acetate, 1 mM DTT). Subsequently, 0.3 Units of T7 exonuclease were added per microgram of DNA and incubated at 37° C. for 30 mins. EDTA was added to a final concentration of 50 mM to terminate the reaction. The resulting products were purified via the DNA Clean & Concentrator system (Zymo Research) and eluted in nuclease-free water. The results are shown in FIG. 2, in lane 10. Inosine is located at −7 position (ITR oligo −267) for lanes 9 & 10.

### Example 4. Endonuclease V Substrates for ssDNA Synthesis

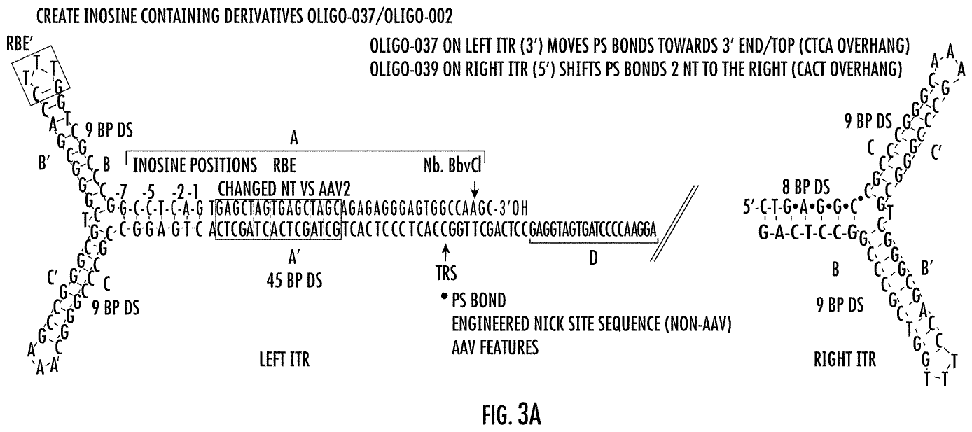

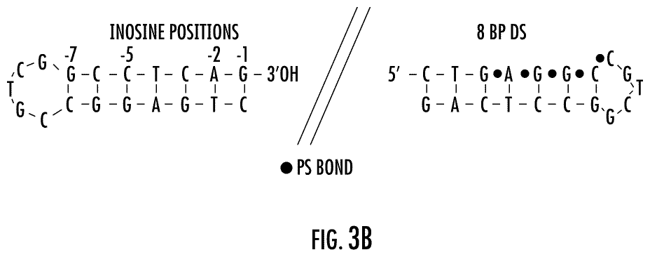

FIG. 3A shows the design of Endonuclease V substrates for single-stranded DNA (ssDNA) synthesis.

The sequences of the inosine-containing ITRs were modeled using Geneious software. This tool uses an energy model that has been previously published (Matthews et al. Proc. Natl. Acad. Sci.

USA, 2004 May 11; 101(19)7287-7292). FIG. 4A shows schematics of the predicted secondary structures of inosine-modified left ITRs. The structure on the far left (i) is the standard (non-modified) structure. The structures designated (ii)-(v) model inosine modification of the left ITR, relative to the 3′ end. Red, green, and blue indicate a high, mid, or low probability of base pairing, respectively. As can be seen in FIG. 4A, the inosine residue at position-5i in (iii) disrupts the secondary structure more than (iv) and (ii). The modeling suggests that the presence of inosine may disrupt base pairing and subsequently the structure of the ITR. This may impact ligation, nicking, or second strand synthesis efficiency. For example, a good priming place for polymerase would not be expected where these instabilities are present.

Oligo-037 (61 bp)
SEQ ID NO: 12
CTCACTGAGGCCGCCCGGGCAAAGCCCGGGCGTCGGGCG
ACCTTTGGTCGCCCGGCCTCAG
Oligo-039 (57 bp)
SEQ ID NO: 13
CACTGAGGCCGCCCGGGCAAAGCCCGGGCGTCGGGCGAC
CTTTGGTCGCCCGGCCTC

FIG. 4B shows the predicted secondary structure of the left ITR after Endonuclease V-mediated ssDNA synthesis. The structures designated (i)-(iv) show the predicted secondary structure of the left ITR, with inosine modification relative to the 3′ end. Red, green, and blue indicate a high, mid, or low probability of base pairing. The 3′ and 5′ end of each ITR is labeled. The modeling suggested that after ssDNA synthesis the structure of the ITR is predicted to change significantly in the case of -5i and -7i. Surprisingly, despite the change in structure for -7i, extension from the 3′OH was observed (see, FIG. 4B). The -5i ITR has a large predicted change in structure that is consistent with the inability of the 3′OH to be extended (see, FIG. 4B). This is likely because the ends of the ITR are predicted to be unpaired. Note for the -1 and -2i the site of digestion occurs in the cargo sequence so no change in the ITR sequence or structure was observed.

Next, the effect of inosine position on second-strand synthesis of ssDNA was examined. RAMP-derived ceDNA were subject to ssDNA synthesis and subsequent second-strand synthesis. For each individual ceDNA, inosine was placed at variable positions relative to the 3′ end of the left ITR. The final base position at the 3′ end of the oligo was designated as -1, and 7 bases upstream of the 3′ end was designated as -7. Inosine was placed at the -1, -2, -5, and -7 position of the ITR. ceDNA lacking inosine (No I) was also generated. Each ceDNA was subjected to Endonuclease V-mediated ssDNA synthesis. Briefly, ceDNA were treated with Endonuclease V (0.75 U/μg) in the presence of 1× NEBuffer 4 (50 mM Potassium acetate, 20 mM Tris-acetate, 10 mM Magnesium Acetate, 1 mM Dithiothreitol (DTT), pH7.9) (New England Biolabs, Inc.) for 2 h at 37° C. Resulting ceDNA were treated with T7 exonuclease (8 units/μg) (New England Biolabs, Inc.) for 30 minutes at 37° C. The reaction was stopped by addition of Ethylenediaminetetraacetic acid (EDTA) to a final concentration of 50 mM. Products were purified using the DNA Clean and Concentrator Kit (Zymo Corporation).

To partially assess ssDNA functionality, purified products were subject to second strand synthesis via DNA Pol I, Large fragment (Klenow exo-) (New England Biolabs, Inc.). Briefly, products were incubated with 0.5 units of Klenow exo- in the presence of 1× NEBuffer 2 (50 mM NaCl, 10 mM Tris-HCl, 10 mM Magnesium Chloride, 1 mM DTT, pH7.5) and 4 mM of each deoxyribonucleotide triphosphates (dNTPs). Samples were incubated for 30 mins at 37° C. The reaction was terminated with addition of EDTA to a final concentration of 80 mM EDTA. RAMP-generated ceDNA, Endonuclease V treated samples, and Klenow exo-samples were analyzed by agarose gel electrophoresis and SYBR gold staining to assess ceDNA quality, ssDNA synthesis, and second-strand synthesis, respectively.

FIG. 5 shows the results of Klenow fill-in of inosine containing single-stranded DNA, demonstrating ssDNA conversion was successful. ceDNA containing no inosine or inosine at various positions within the left ITR were generated via RAMP (lanes 2, 5, 8, 11, 14). ceDNA were subjected to Endonuclease V-mediated ssDNA synthesis (lanes 3, 7, 10, 15). The resulting products were treated with DNA polymerase I large (Klenow) fragment exo- to facilitate second-strand synthesis (lanes 4, 7, 10, 13, 16). Note that ceDNA and products of successful second-strand synthesis of ssDNA co-migrate. Residual ceDNA that was left over indicates how inefficient the conversion was. FIG. 5 shows successful ssDNA conversion. The results from FIG. 5 suggested that -1i and -7i were candidates for moving forward.

Next, the Endonuclease V-mediated ssDNA synthesis approach was applied to a diverse set of ceDNA to test universality of the approach. Multiple ceDNA were generated via RAMP. Each ceDNA encoded unique internal sequences and harbored the left ITR containing inosine at the −1 position and a right ITR with an extended A-stem (SO-238; SEQ ID NO: 14). Control ceDNA lacked inosine in the right ITR. To produce ssDNA, all ceDNA were treated with Endonuclease V (New England Biolabs, Inc.) (2 units/μg) for 2 h at 37° C. in the presence of 1× NEBuffer 4. DTT was added to a final concentration of 1 mM. Samples were treated with 21 units/μg T7 exonuclease (New England Biolabs, Inc.). EDTA was added to 50 mM to terminate the reaction. Products were purified via the DNA clean and concentrator kit (Zymo Corporation).

TABLE 8

| Lane | | | | ssDNA (if |
|---|---|---|---|---|
| number | Description | Reaction | ceDNA | applicable) |
| 1 | Size marker | NA | NA | NA |
| 2 | No inosine contained within ceDNA | RAMP (ceDNA) | 710 | NA |
| 3 | No inosine contained within ceDNA | ssDNA synthesis | 710 | NA |
| 4 | No inosine contained within ceDNA | DNA polymerase fill | 710 | NA |
| | | in (Klenow exo-) | | |
| 5 | Inosine at −1 position of left ITR | RAMP (ceDNA) | 711 | NA |
| | within ceDNA | | | |
| 6 | Inosine at −1 position of left ITR | SsDNA synthesis | 711 | 084 |
| | within ceDNA | | | |
| 7 | Inosine at −1 position of left ITR | DNA polymerase fill | 711 | 084 |
| | within ceDNA | in (Klenow exo-) | | |
| 8 | Inosine at −2 position of left ITR | RAMP (ceDNA) | 712 | NA |
| | within ceDNA | | | |
| 9 | Inosine at −2 position of left ITR | ssDNA synthesis | 712 | 086 |
| | within ceDNA | | | |
| 10 | Inosine at −2 position of left ITR | DNA polymerase fill | 712 | 086 |
| | within ceDNA | in (Klenow exo-) | | |
| 11 | Inosine at −5 position of left ITR | RAMP (ceDNA) | 713 | NA |
| | within ceDNA | | | |
| 12 | Inosine at −5 position of left ITR | ssDNA synthesis | 713 | 088 |
| | within ceDNA | | | |
| 13 | Inosine at −5 position of left ITR | DNA polymerase fill | 713 | 088 |
| | within ceDNA | in (Klenow exo-) | | |
| 14 | Inosine at −7 position of left ITR | RAMP (ceDNA) | 656 | NA |
| | within ceDNA | | | |
| 15 | Inosine at −7 position of left ITR | ssDNA synthesis | 656 | 042 |
| | within ceDNA | | | |
| 16 | Inosine at −7 position of left ITR | DNA polymerase fill | 656 | 042 |
| | within ceDNA | in (Klenow exo-) | | |

710 (Lanes 2-4; 6206 bp; hFVIII ORF 831-5222)
CTGAGGCCGCCCGGGCAAAGCCCGGGCGTCGGGCGACCTTTGGTCGCCCGGCCTCAGTGAGCGAGCGA
GCGCGCAGAGAGGGAGTGGCCAAGCTGAGGCTCCATCACTAGGGGTTCCTTGTAGTTAATGATTAACC
CGCCATGCTACTTATCGCGGCCGCGGGGGAGGCTGCTGGTGAATATTAACCAAGGTCACCCCAGTTAT
CGGAGGAGCAAACAGGGGCTAAGTCCACCGGGGGAGGCTGCTGGTGAATATTAACCAAGGTCACCCCA
GTTATCGGAGGAGCAAACAGGGGCTAAGTCCACCGGGGGAGGCTGCTGGTGAATATTAACCAAGGTCA
CCCCAGTTATCGGAGGAGCAAACAGGGGCTAAGTCCACGGTACCCACTGGGAGGATGTTGAGTAAGAT
GGAAAACTACTGATGACCCTTGCAGAGACAGAGTATTAGGACATGTTTGAACAGGGGCCGGGCGATCA
GCAGGTAGCTCTAGAGGATCCCCGTCTGTCTGCACATTTCGTAGAGCGAGTGTTCCGATACTCTAATC
TCCCTAGGCAAGGTTCATATTTGTGTAGGTTACTTATTCTCCTTTTGTTGACTAAGTCAATAATCAGA
ATCAGCAGGTTTGGAGTCAGCTTGGCAGGGATCAGCAGCCTGGGTTGGAAGGAGGGGGTATAAAAGCC
CCTTCACCAGGAGAAGCCGTCACACAGATCCACAAGCTCCTGAAGAGGTAAGGGTTTAAGGGATGGTT
GGTTGGTGGGGTATTAATGTTTAATTACCTGGAGCACCTGCCTGAAATCACTTTTTTTCAGGTTGGTT
TAAACGCCGCCACCATGCAGATTGAGCTGAGCACCTGCTTCTTCCTGTGCCTGCTGAGGTTCTGCTTC
TCTGCCACCAGGAGATACTACCTGGGGGCTGTGGAGCTGAGCTGGGACTACATGCAGTCTGACCTGGG
GGAGCTGCCTGTGGATGCCAGGTTCCCCCCCAGAGTGCCCAAGAGCTTCCCCTTCAACACCTCTGTGG
TGTACAAGAAGACCCTGTTTGTGGAGTTCACTGACCACCTGTTCAACATTGCCAAGCCCAGGCCCCCC
TGGATGGGCCTGCTGGGCCCCACCATCCAGGCTGAGGTGTATGACACTGTGGTGATCACCCTGAAGAA
CATGGCCAGCCACCCTGTGAGCCTGCATGCTGTGGGGGTGAGCTACTGGAAGGCCTCTGAGGGGGCTG
AGTATGATGACCAGACCAGCCAGAGGGAGAAGGAGGATGACAAGGTGTTCCCTGGGGGCAGCCACACC
TATGTGTGGCAGGTGCTGAAGGAGAATGGCCCCATGGCCTCTGACCCCCTGTGCCTGACCTACAGCTA
CCTGAGCCATGTGGACCTGGTGAAGGACCTGAACTCTGGCCTGATTGGGGCCCTGCTGGTGTGCAGGG
AGGGCAGCCTGGCCAAGGAGAAGACCCAGACCCTGCACAAGTTCATCCTGCTGTTTGCTGTGTTTGAT
GAGGGCAAGAGCTGGCACTCTGAAACCAAGAACAGCCTGATGCAGGACAGGGATGCTGCCTCTGCCAG
GGCCTGGCCCAAGATGCACACTGTGAATGGCTATGTGAACAGGAGCCTGCCTGGCCTGATTGGCTGCC
ACAGGAAGTCTGTGTACTGGCATGTGATTGGCATGGGCACCACCCCTGAGGTGCACAGCATCTTCCTG
GAGGGCCACACCTTCCTGGTCAGGAACCACAGGCAGGCCAGCCTGGAGATCAGCCCCATCACCTTCCT
GACTGCCCAGACCCTGCTGATGGACCTGGGCCAGTTCCTGCTGTTCTGCCACATCAGCAGCCACCAGC
ATGATGGCATGGAGGCCTATGTGAAGGTGGACAGCTGCCCTGAGGAGCCCCAGCTGAGGATGAAGAAC
AATGAGGAGGCTGAGGACTATGATGATGACCTGACTGACTCTGAGATGGATGTGGTGAGGTTTGATGA
TGACAACAGCCCCAGCTTCATCCAGATCAGGTCTGTGGCCAAGAAGCACCCCAAGACCTGGGTGCACT
ACATTGCTGCTGAGGAGGAGGACTGGGACTATGCCCCCCTGGTGCTGGCCCCTGATGACAGGAGCTAC
AAGAGCCAGTACCTGAACAATGGCCCCCAGAGGATTGGCAGGAAGTACAAGAAGGTCAGGTTCATGGC
CTACACTGATGAAACCTTCAAGACCAGGGAGGCCATCCAGCATGAGTCTGGCATCCTGGGCCCCCTGC
TGTATGGGGAGGTGGGGGACACCCTGCTGATCATCTTCAAGAACCAGGCCAGCAGGCCCTACAACATC
TACCCCCATGGCATCACTGATGTGAGGCCCCTGTACAGCAGGAGGCTGCCCAAGGGGGTGAAGCACCT
GAAGGACTTCCCCATCCTGCCTGGGGAGATCTTCAAGTACAAGTGGACTGTGACTGTGGAGGATGGCC
CCACCAAGTCTGACCCCAGGTGCCTGACCAGATACTACAGCAGCTTTGTGAACATGGAGAGGGACCTG
GCCTCTGGCCTGATTGGCCCCCTGCTGATCTGCTACAAGGAGTCTGTGGACCAGAGGGGCAACCAGAT
CATGTCTGACAAGAGGAATGTGATCCTGTTCTCTGTGTTTGATGAGAACAGGAGCTGGTACCTGACTG
AGAACATCCAGAGGTTCCTGCCCAACCCTGCTGGGGTGCAGCTGGAGGACCCTGAGTTCCAGGCCAGC
AACATCATGCACAGCATCAATGGCTATGTGTTTGACAGCCTGCAGCTGTCTGTGTGCCTGCATGAGGT
GGCCTACTGGTACATCCTGAGCATTGGGGCCCAGACTGACTTCCTGTCTGTGTTCTTCTCTGGCTACA
CCTTCAAGCACAAGATGGTGTATGAGGACACCCTGACCCTGTTCCCCTTCTCTGGGGAGACTGTGTTC
ATGAGCATGGAGAACCCTGGCCTGTGGATTCTGGGCTGCCACAACTCTGACTTCAGGAACAGGGGCAT
GACTGCCCTGCTGAAAGTCTCCAGCTGTGACAAGAACACTGGGGACTACTATGAGGACAGCTATGAGG
ACATCTCTGCCTACCTGCTGAGCAAGAACAATGCCATTGAGCCCAGGAGCTTCAGCCAGAATAGCAGG
CACCCCAGCACCAGGCAGAAGCAGTTCAATGCCACCACCATCCCAGAGAATACCACCCTGCAGTCTGA
CCAGGAGGAGATTGACTATGATGACACCATCTCTGTGGAGATGAAGAAGGAGGACTTTGACATCTACG
ACGAGGACGAGAACCAGAGCCCCAGGAGCTTCCAGAAGAAGACCAGGCACTACTTCATTGCTGCTGTG
GAGAGGCTGTGGGACTATGGCATGAGCAGCAGCCCCCATGTGCTGAGGAACAGGGCCCAGTCTGGCTC
TGTGCCCCAGTTCAAGAAGGTGGTGTTCCAGGAGTTCACTGATGGCAGCTTCACCCAGCCCCTGTACA
GAGGGGAGCTGAATGAGCACCTGGGCCTGCTGGGCCCCTACATCAGGGCTGAGGTGGAGGACAACATC
ATGGTGACCTTCAGGAACCAGGCCAGCAGGCCCTACAGCTTCTACAGCAGCCTGATCAGCTATGAGGA
GGACCAGAGGCAGGGGGCTGAGCCCAGGAAGAACTTTGTGAAGCCCAATGAAACCAAGACCTACTTCT
GGAAGGTGCAGCACCACATGGCCCCCACCAAGGATGAGTTTGACTGCAAGGCCTGGGCCTACTTCTCT
GATGTGGACCTGGAGAAGGATGTGCACTCTGGCCTGATTGGCCCCCTGCTGGTGTGCCACACCAACAC
CCTGAACCCTGCCCATGGCAGGCAGGTGACTGTGCAGGAGTTTGCCCTGTTCTTCACCATCTTTGATG
AAACCAAGAGCTGGTACTTCACTGAGAACATGGAGAGGAACTGCAGGGCCCCCTGCAACATCCAGATG
GAGGACCCCACCTTCAAGGAGAACTACAGGTTCCATGCCATCAATGGCTACATCATGGACACCCTGCC
TGGCCTGGTGATGGCCCAGGACCAGAGGATCAGGTGGTACCTGCTGAGCATGGGCAGCAATGAGAACA
TCCACAGCATCCACTTCTCTGGCCATGTGTTCACTGTGAGGAAGAAGGAGGAGTACAAGATGGCCCTG
TACAACCTGTACCCTGGGGTGTTTGAGACTGTGGAGATGCTGCCCAGCAAGGCTGGCATCTGGAGGGT
GGAGTGCCTGATTGGGGAGCACCTGCATGCTGGCATGAGCACCCTGTTCCTGGTGTACAGCAACAAGT
GCCAGACCCCCCTGGGCATGGCCTCTGGCCACATCAGGGACTTCCAGATCACTGCCTCTGGCCAGTAT
GGCCAGTGGGCCCCCAAGCTGGCCAGGCTGCACTACTCTGGCAGCATCAATGCCTGGAGCACCAAGGA
GCCCTTCAGCTGGATCAAGGTGGACCTGCTGGCCCCCATGATCATCCATGGCATCAAGACCCAGGGGG
CCAGGCAGAAGTTCAGCAGCCTGTACATCAGCCAGTTCATCATCATGTACAGCCTGGATGGCAAGAAG
TGGCAGACCTACAGGGGCAACAGCACTGGCACCCTGATGGTGTTCTTTGGCAATGTGGACAGCTCTGG
CATCAAGCACAACATCTTCAACCCCCCCATCATTGCCAGATACATCAGGCTGCACCCCACCCACTACA
GCATCAGGAGCACCCTGAGGATGGAGCTGATGGGCTGTGACCTGAACAGCTGCAGCATGCCCCTGGGC
ATGGAGAGCAAGGCCATCTCTGATGCCCAGATCACTGCCAGCAGCTACTTCACCAACATGTTTGCCAC
CTGGAGCCCCAGCAAGGCCAGGCTGCACCTGCAGGGCAGGAGCAATGCCTGGAGGCCCCAGGTCAACA
ACCCCAAGGAGTGGCTGCAGGTGGACTTCCAGAAGACCATGAAGGTGACTGGGGTGACCACCCAGGGG
GTGAAGAGCCTGCTGACCAGCATGTATGTGAAGGAGTTCCTGATCAGCAGCAGCCAGGATGGCCACCA
GTGGACCCTGTTCTTCCAGAATGGCAAGGTGAAGGTGTTCCAGGGCAACCAGGACAGCTTCACCCCTG
TGGTGAACAGCCTGGACCCCCCCCTGCTGACCAGATACCTGAGGATTCACCCCCAGAGCTGGGTGCAC
CAGATTGCCCTGAGGATGGAGGTGCTGGGCTGTGAGGCCCAGGACCTGTACTGATTAATTAAGAGCAT
CTTACCGCCATTTATTCCCATATTTGTTCTGTTTTTCTTGATTTGGGTATACATTTAAATGTTAATAA
AACAAAATGGTGGGGCAATCATTTACATTTTTAGGGATATGTAATTACTAGTTCAGGTGTATTGCCAC
AAGACAAACATGTTAAGAAACTTTCCCGTTATTTACGCTCTGTTCCTGTTAATCAACCTCTGGATTAC
AAAATTTGTGAAAGATTGACTGATATTCTTAACTATGTTGCTCCTTTTACGCTGTGTGGATATGCTGC
TTTATAGCCTCTGTATCTAGCTATTGCTTCCCGTACGGCTTTCGTTTTCTCCTCCTTGTATAAATCCT
GGTTGCTGTCTCTTTTAGAGGAGTTGTGGCCCGTTGTCCGTCAACGTGGCGTGGTGTGCTCTGTGTTT
GCTGACGCAACCCCCACTGGCTGGGGCATTGCCACCACCTGTCAACTCCTTTCTGGGACTTTCGCTTT
CCCCCTCCCGATCGCCACGGCAGAACTCATCGCCGCCTGCCTTGCCCGCTGCTGGACAGGGGCTAGGT
TGCTGGGCACTGATAATTCCGTGGTGTTGTCTGTGCCTTCTAGTTGCCAGCCATCTGTTGTTTGCCCC
TCCCCCGTGCCTTCCTTGACCCTGGAAGGTGCCACTCCCACTGTCCTTTCCTAATAAAATGAGGAAAT
TGCATCGCATTGTCTGAGTAGGTGTCATTCTATTCTGGGGGGTGGGGTGGGGCAGGACAGCAAGGGGG
AGGATTGGGAAGACAATAGCAGGCATGCTGGGGATGCGGTGGGCTCTATGGCTCTAGAGCATGGCTAC
GTAGATAAGTAGCATGGCGGGTTAATCATTAACTACACCTGCAGGAGGAACCCCTAGTGATGGAGTTG
GCCACTCCCTCTCTGCGCGCTCGCTCGCTCACTGAGGCCGCCCGGGCAAAGCCCGGGCGTCGGGCGAC
CTTTGGTCGCCCGGCCTC
711 (Lanes 5-7; 6206 bp; hFVIII ORF 831-5222)
CTGAGGCCGCCCGGGCAAAGCCCGGGCGTCGGGCGACCTTTGGTCGCCCGGCCTCANTGAGCGAGCGA
GCGCGCAGAGAGGGAGTGGCCAAGCTGAGGCTCCATCACTAGGGGTTCCTTGTAGTTAATGATTAACC
CGCCATGCTACTTATCGCGGCCGCGGGGGAGGCTGCTGGTGAATATTAACCAAGGTCACCCCAGTTAT
CGGAGGAGCAAACAGGGGCTAAGTCCACCGGGGGAGGCTGCTGGTGAATATTAACCAAGGTCACCCCA
GTTATCGGAGGAGCAAACAGGGGCTAAGTCCACCGGGGGAGGCTGCTGGTGAATATTAACCAAGGTCA
CCCCAGTTATCGGAGGAGCAAACAGGGGCTAAGTCCACGGTACCCACTGGGAGGATGTTGAGTAAGAT
GGAAAACTACTGATGACCCTTGCAGAGACAGAGTATTAGGACATGTTTGAACAGGGGCCGGGCGATCA
GCAGGTAGCTCTAGAGGATCCCCGTCTGTCTGCACATTTCGTAGAGCGAGTGTTCCGATACTCTAATC
TCCCTAGGCAAGGTTCATATTTGTGTAGGTTACTTATTCTCCTTTTGTTGACTAAGTCAATAATCAGA
ATCAGCAGGTTTGGAGTCAGCTTGGCAGGGATCAGCAGCCTGGGTTGGAAGGAGGGGGTATAAAAGCC
CCTTCACCAGGAGAAGCCGTCACACAGATCCACAAGCTCCTGAAGAGGTAAGGGTTTAAGGGATGGTT
GGTTGGTGGGGTATTAATGTTTAATTACCTGGAGCACCTGCCTGAAATCACTTTTTTTCAGGTTGGTT
TAAACGCCGCCACCATGCAGATTGAGCTGAGCACCTGCTTCTTCCTGTGCCTGCTGAGGTTCTGCTTC
TCTGCCACCAGGAGATACTACCTGGGGGCTGTGGAGCTGAGCTGGGACTACATGCAGTCTGACCTGGG
GGAGCTGCCTGTGGATGCCAGGTTCCCCCCCAGAGTGCCCAAGAGCTTCCCCTTCAACACCTCTGTGG
TGTACAAGAAGACCCTGTTTGTGGAGTTCACTGACCACCTGTTCAACATTGCCAAGCCCAGGCCCCCC
TGGATGGGCCTGCTGGGCCCCACCATCCAGGCTGAGGTGTATGACACTGTGGTGATCACCCTGAAGAA
CATGGCCAGCCACCCTGTGAGCCTGCATGCTGTGGGGGTGAGCTACTGGAAGGCCTCTGAGGGGGCTG
AGTATGATGACCAGACCAGCCAGAGGGAGAAGGAGGATGACAAGGTGTTCCCTGGGGGCAGCCACACC
TATGTGTGGCAGGTGCTGAAGGAGAATGGCCCCATGGCCTCTGACCCCCTGTGCCTGACCTACAGCTA
CCTGAGCCATGTGGACCTGGTGAAGGACCTGAACTCTGGCCTGATTGGGGCCCTGCTGGTGTGCAGGG
AGGGCAGCCTGGCCAAGGAGAAGACCCAGACCCTGCACAAGTTCATCCTGCTGTTTGCTGTGTTTGAT
GAGGGCAAGAGCTGGCACTCTGAAACCAAGAACAGCCTGATGCAGGACAGGGATGCTGCCTCTGCCAG
GGCCTGGCCCAAGATGCACACTGTGAATGGCTATGTGAACAGGAGCCTGCCTGGCCTGATTGGCTGCC
ACAGGAAGTCTGTGTACTGGCATGTGATTGGCATGGGCACCACCCCTGAGGTGCACAGCATCTTCCTG
GAGGGCCACACCTTCCTGGTCAGGAACCACAGGCAGGCCAGCCTGGAGATCAGCCCCATCACCTTCCT
GACTGCCCAGACCCTGCTGATGGACCTGGGCCAGTTCCTGCTGTTCTGCCACATCAGCAGCCACCAGC
ATGATGGCATGGAGGCCTATGTGAAGGTGGACAGCTGCCCTGAGGAGCCCCAGCTGAGGATGAAGAAC
AATGAGGAGGCTGAGGACTATGATGATGACCTGACTGACTCTGAGATGGATGTGGTGAGGTTTGATGA
TGACAACAGCCCCAGCTTCATCCAGATCAGGTCTGTGGCCAAGAAGCACCCCAAGACCTGGGTGCACT
ACATTGCTGCTGAGGAGGAGGACTGGGACTATGCCCCCCTGGTGCTGGCCCCTGATGACAGGAGCTAC
AAGAGCCAGTACCTGAACAATGGCCCCCAGAGGATTGGCAGGAAGTACAAGAAGGTCAGGTTCATGGC
CTACACTGATGAAACCTTCAAGACCAGGGAGGCCATCCAGCATGAGTCTGGCATCCTGGGCCCCCTGC
TGTATGGGGAGGTGGGGGACACCCTGCTGATCATCTTCAAGAACCAGGCCAGCAGGCCCTACAACATC
TACCCCCATGGCATCACTGATGTGAGGCCCCTGTACAGCAGGAGGCTGCCCAAGGGGGTGAAGCACCT
GAAGGACTTCCCCATCCTGCCTGGGGAGATCTTCAAGTACAAGTGGACTGTGACTGTGGAGGATGGCC
CCACCAAGTCTGACCCCAGGTGCCTGACCAGATACTACAGCAGCTTTGTGAACATGGAGAGGGACCTG
GCCTCTGGCCTGATTGGCCCCCTGCTGATCTGCTACAAGGAGTCTGTGGACCAGAGGGGCAACCAGAT
CATGTCTGACAAGAGGAATGTGATCCTGTTCTCTGTGTTTGATGAGAACAGGAGCTGGTACCTGACTG
AGAACATCCAGAGGTTCCTGCCCAACCCTGCTGGGGTGCAGCTGGAGGACCCTGAGTTCCAGGCCAGC
AACATCATGCACAGCATCAATGGCTATGTGTTTGACAGCCTGCAGCTGTCTGTGTGCCTGCATGAGGT
GGCCTACTGGTACATCCTGAGCATTGGGGCCCAGACTGACTTCCTGTCTGTGTTCTTCTCTGGCTACA
CCTTCAAGCACAAGATGGTGTATGAGGACACCCTGACCCTGTTCCCCTTCTCTGGGGAGACTGTGTTC
ATGAGCATGGAGAACCCTGGCCTGTGGATTCTGGGCTGCCACAACTCTGACTTCAGGAACAGGGGCAT
GACTGCCCTGCTGAAAGTCTCCAGCTGTGACAAGAACACTGGGGACTACTATGAGGACAGCTATGAGG
ACATCTCTGCCTACCTGCTGAGCAAGAACAATGCCATTGAGCCCAGGAGCTTCAGCCAGAATAGCAGG
CACCCCAGCACCAGGCAGAAGCAGTTCAATGCCACCACCATCCCAGAGAATACCACCCTGCAGTCTGA
CCAGGAGGAGATTGACTATGATGACACCATCTCTGTGGAGATGAAGAAGGAGGACTTTGACATCTACG
ACGAGGACGAGAACCAGAGCCCCAGGAGCTTCCAGAAGAAGACCAGGCACTACTTCATTGCTGCTGTG
GAGAGGCTGTGGGACTATGGCATGAGCAGCAGCCCCCATGTGCTGAGGAACAGGGCCCAGTCTGGCTC
TGTGCCCCAGTTCAAGAAGGTGGTGTTCCAGGAGTTCACTGATGGCAGCTTCACCCAGCCCCTGTACA
GAGGGGAGCTGAATGAGCACCTGGGCCTGCTGGGCCCCTACATCAGGGCTGAGGTGGAGGACAACATC
ATGGTGACCTTCAGGAACCAGGCCAGCAGGCCCTACAGCTTCTACAGCAGCCTGATCAGCTATGAGGA
GGACCAGAGGCAGGGGGCTGAGCCCAGGAAGAACTTTGTGAAGCCCAATGAAACCAAGACCTACTTCT
GGAAGGTGCAGCACCACATGGCCCCCACCAAGGATGAGTTTGACTGCAAGGCCTGGGCCTACTTCTCT
GATGTGGACCTGGAGAAGGATGTGCACTCTGGCCTGATTGGCCCCCTGCTGGTGTGCCACACCAACAC
CCTGAACCCTGCCCATGGCAGGCAGGTGACTGTGCAGGAGTTTGCCCTGTTCTTCACCATCTTTGATG
AAACCAAGAGCTGGTACTTCACTGAGAACATGGAGAGGAACTGCAGGGCCCCCTGCAACATCCAGATG
GAGGACCCCACCTTCAAGGAGAACTACAGGTTCCATGCCATCAATGGCTACATCATGGACACCCTGCC
TGGCCTGGTGATGGCCCAGGACCAGAGGATCAGGTGGTACCTGCTGAGCATGGGCAGCAATGAGAACA
TCCACAGCATCCACTTCTCTGGCCATGTGTTCACTGTGAGGAAGAAGGAGGAGTACAAGATGGCCCTG
TACAACCTGTACCCTGGGGTGTTTGAGACTGTGGAGATGCTGCCCAGCAAGGCTGGCATCTGGAGGGT
GGAGTGCCTGATTGGGGAGCACCTGCATGCTGGCATGAGCACCCTGTTCCTGGTGTACAGCAACAAGT
GCCAGACCCCCCTGGGCATGGCCTCTGGCCACATCAGGGACTTCCAGATCACTGCCTCTGGCCAGTAT
GGCCAGTGGGCCCCCAAGCTGGCCAGGCTGCACTACTCTGGCAGCATCAATGCCTGGAGCACCAAGGA
GCCCTTCAGCTGGATCAAGGTGGACCTGCTGGCCCCCATGATCATCCATGGCATCAAGACCCAGGGGG
CCAGGCAGAAGTTCAGCAGCCTGTACATCAGCCAGTTCATCATCATGTACAGCCTGGATGGCAAGAAG
TGGCAGACCTACAGGGGCAACAGCACTGGCACCCTGATGGTGTTCTTTGGCAATGTGGACAGCTCTGG
CATCAAGCACAACATCTTCAACCCCCCCATCATTGCCAGATACATCAGGCTGCACCCCACCCACTACA
GCATCAGGAGCACCCTGAGGATGGAGCTGATGGGCTGTGACCTGAACAGCTGCAGCATGCCCCTGGGC
ATGGAGAGCAAGGCCATCTCTGATGCCCAGATCACTGCCAGCAGCTACTTCACCAACATGTTTGCCAC
CTGGAGCCCCAGCAAGGCCAGGCTGCACCTGCAGGGCAGGAGCAATGCCTGGAGGCCCCAGGTCAACA
ACCCCAAGGAGTGGCTGCAGGTGGACTTCCAGAAGACCATGAAGGTGACTGGGGTGACCACCCAGGGG
GTGAAGAGCCTGCTGACCAGCATGTATGTGAAGGAGTTCCTGATCAGCAGCAGCCAGGATGGCCACCA
GTGGACCCTGTTCTTCCAGAATGGCAAGGTGAAGGTGTTCCAGGGCAACCAGGACAGCTTCACCCCTG
TGGTGAACAGCCTGGACCCCCCCCTGCTGACCAGATACCTGAGGATTCACCCCCAGAGCTGGGTGCAC
CAGATTGCCCTGAGGATGGAGGTGCTGGGCTGTGAGGCCCAGGACCTGTACTGATTAATTAAGAGCAT
CTTACCGCCATTTATTCCCATATTTGTTCTGTTTTTCTTGATTTGGGTATACATTTAAATGTTAATAA
AACAAAATGGTGGGGCAATCATTTACATTTTTAGGGATATGTAATTACTAGTTCAGGTGTATTGCCAC
AAGACAAACATGTTAAGAAACTTTCCCGTTATTTACGCTCTGTTCCTGTTAATCAACCTCTGGATTAC
AAAATTTGTGAAAGATTGACTGATATTCTTAACTATGTTGCTCCTTTTACGCTGTGTGGATATGCTGC
TTTATAGCCTCTGTATCTAGCTATTGCTTCCCGTACGGCTTTCGTTTTCTCCTCCTTGTATAAATCCT
GGTTGCTGTCTCTTTTAGAGGAGTTGTGGCCCGTTGTCCGTCAACGTGGCGTGGTGTGCTCTGTGTTT
GCTGACGCAACCCCCACTGGCTGGGGCATTGCCACCACCTGTCAACTCCTTTCTGGGACTTTCGCTTT
CCCCCTCCCGATCGCCACGGCAGAACTCATCGCCGCCTGCCTTGCCCGCTGCTGGACAGGGGCTAGGT
TGCTGGGCACTGATAATTCCGTGGTGTTGTCTGTGCCTTCTAGTTGCCAGCCATCTGTTGTTTGCCCC
TCCCCCGTGCCTTCCTTGACCCTGGAAGGTGCCACTCCCACTGTCCTTTCCTAATAAAATGAGGAAAT
TGCATCGCATTGTCTGAGTAGGTGTCATTCTATTCTGGGGGGTGGGGTGGGGCAGGACAGCAAGGGGG
AGGATTGGGAAGACAATAGCAGGCATGCTGGGGATGCGGTGGGCTCTATGGCTCTAGAGCATGGCTAC
GTAGATAAGTAGCATGGCGGGTTAATCATTAACTACACCTGCAGGAGGAACCCCTAGTGATGGAGTTG
GCCACTCCCTCTCTGCGCGCTCGCTCGCTCACTGAGGCCGCCCGGGCAAAGCCCGGGCGTCGGGCGAC
CTTTGGTCGCCCGGCCTC
084 (Lanes 6-7; 6207 bp; hFVIII ORF 985-5376))
GAGGCCGCCCGGGCAAAGCCCGGGCGTCGGGCGACCTTTGGTCGCCCGGCCTCAGTGAGCGAGCGAGC
GCGCAGAGAGGGAGTGGCCAACTCCATCACTAGGGGTTCCTCCTGCAGGTGTAGTTAATGATTAACCC
GCCATGCTACTTATCTACGTAGCCATGCTCTAGAGCCATAGAGCCCACCGCATCCCCAGCATGCCTGC
TATTGTCTTCCCAATCCTCCCCCTTGCTGTCCTGCCCCACCCCACCCCCCAGAATAGAATGACACCTA
CTCAGACAATGCGATGCAATTTCCTCATTTTATTAGGAAAGGACAGTGGGAGTGGCACCTTCCAGGGT
CAAGGAAGGCACGGGGGAGGGGCAAACAACAGATGGCTGGCAACTAGAAGGCACAGACAACACCACGG
AATTATCAGTGCCCAGCAACCTAGCCCCTGTCCAGCAGCGGGCAAGGCAGGCGGCGATGAGTTCTGCC
GTGGCGATCGGGAGGGGGAAAGCGAAAGTCCCAGAAAGGAGTTGACAGGTGGTGGCAATGCCCCAGCC
AGTGGGGGTTGCGTCAGCAAACACAGAGCACACCACGCCACGTTGACGGACAACGGGCCACAACTCCT
CTAAAAGAGACAGCAACCAGGATTTATACAAGGAGGAGAAAACGAAAGCCGTACGGGAAGCAATAGCT
AGATACAGAGGCTATAAAGCAGCATATCCACACAGCGTAAAAGGAGCAACATAGTTAAGAATATCAGT
CAATCTTTCACAAATTTTGTAATCCAGAGGTTGATTAACAGGAACAGAGCGTAAATAACGGGAAAGTT
TCTTAACATGTTTGTCTTGTGGCAATACACCTGAACTAGTAATTACATATCCCTAAAAATGTAAATGA
TTGCCCCACCATTTTGTTTTATTAACATTTAAATGTATACCCAAATCAAGAAAAACAGAACAAATATG
GGAATAAATGGCGGTAAGATGCTCTTAATTAATCAGTACAGGTCCTGGGCCTCACAGCCCAGCACCTC
CATCCTCAGGGCAATCTGGTGCACCCAGCTCTGGGGGTGAATCCTCAGGTATCTGGTCAGCAGGGGGG
GGTCCAGGCTGTTCACCACAGGGGTGAAGCTGTCCTGGTTGCCCTGGAACACCTTCACCTTGCCATTC
TGGAAGAACAGGGTCCACTGGTGGCCATCCTGGCTGCTGCTGATCAGGAACTCCTTCACATACATGCT
GGTCAGCAGGCTCTTCACCCCCTGGGTGGTCACCCCAGTCACCTTCATGGTCTTCTGGAAGTCCACCT
GCAGCCACTCCTTGGGGTTGTTGACCTGGGGCCTCCAGGCATTGCTCCTGCCCTGCAGGTGCAGCCTG
GCCTTGCTGGGGCTCCAGGTGGCAAACATGTTGGTGAAGTAGCTGCTGGCAGTGATCTGGGCATCAGA
GATGGCCTTGCTCTCCATGCCCAGGGGCATGCTGCAGCTGTTCAGGTCACAGCCCATCAGCTCCATCC
TCAGGGTGCTCCTGATGCTGTAGTGGGTGGGGTGCAGCCTGATGTATCTGGCAATGATGGGGGGGTTG
AAGATGTTGTGCTTGATGCCAGAGCTGTCCACATTGCCAAAGAACACCATCAGGGTGCCAGTGCTGTT
GCCCCTGTAGGTCTGCCACTTCTTGCCATCCAGGCTGTACATGATGATGAACTGGCTGATGTACAGGC
TGCTGAACTTCTGCCTGGCCCCCTGGGTCTTGATGCCATGGATGATCATGGGGGCCAGCAGGTCCACC
TTGATCCAGCTGAAGGGCTCCTTGGTGCTCCAGGCATTGATGCTGCCAGAGTAGTGCAGCCTGGCCAG
CTTGGGGGCCCACTGGCCATACTGGCCAGAGGCAGTGATCTGGAAGTCCCTGATGTGGCCAGAGGCCA
TGCCCAGGGGGGTCTGGCACTTGTTGCTGTACACCAGGAACAGGGTGCTCATGCCAGCATGCAGGTGC
TCCCCAATCAGGCACTCCACCCTCCAGATGCCAGCCTTGCTGGGCAGCATCTCCACAGTCTCAAACAC
CCCAGGGTACAGGTTGTACAGGGCCATCTTGTACTCCTCCTTCTTCCTCACAGTGAACACATGGCCAG
AGAAGTGGATGCTGTGGATGTTCTCATTGCTGCCCATGCTCAGCAGGTACCACCTGATCCTCTGGTCC
TGGGCCATCACCAGGCCAGGCAGGGTGTCCATGATGTAGCCATTGATGGCATGGAACCTGTAGTTCTC
CTTGAAGGTGGGGTCCTCCATCTGGATGTTGCAGGGGGCCCTGCAGTTCCTCTCCATGTTCTCAGTGA
AGTACCAGCTCTTGGTTTCATCAAAGATGGTGAAGAACAGGGCAAACTCCTGCACAGTCACCTGCCTG
CCATGGGCAGGGTTCAGGGTGTTGGTGTGGCACACCAGCAGGGGGCCAATCAGGCCAGAGTGCACATC
CTTCTCCAGGTCCACATCAGAGAAGTAGGCCCAGGCCTTGCAGTCAAACTCATCCTTGGTGGGGGCCA
TGTGGTGCTGCACCTTCCAGAAGTAGGTCTTGGTTTCATTGGGCTTCACAAAGTTCTTCCTGGGCTCA
GCCCCCTGCCTCTGGTCCTCCTCATAGCTGATCAGGCTGCTGTAGAAGCTGTAGGGCCTGCTGGCCTG
GTTCCTGAAGGTCACCATGATGTTGTCCTCCACCTCAGCCCTGATGTAGGGGCCCAGCAGGCCCAGGT
GCTCATTCAGCTCCCCTCTGTACAGGGGCTGGGTGAAGCTGCCATCAGTGAACTCCTGGAACACCACC
TTCTTGAACTGGGGCACAGAGCCAGACTGGGCCCTGTTCCTCAGCACATGGGGGCTGCTGCTCATGCC
ATAGTCCCACAGCCTCTCCACAGCAGCAATGAAGTAGTGCCTGGTCTTCTTCTGGAAGCTCCTGGGGC
TCTGGTTCTCGTCCTCGTCGTAGATGTCAAAGTCCTCCTTCTTCATCTCCACAGAGATGGTGTCATCA
TAGTCAATCTCCTCCTGGTCAGACTGCAGGGTGGTATTCTCTGGGATGGTGGTGGCATTGAACTGCTT
CTGCCTGGTGCTGGGGTGCCTGCTATTCTGGCTGAAGCTCCTGGGCTCAATGGCATTGTTCTTGCTCA
GCAGGTAGGCAGAGATGTCCTCATAGCTGTCCTCATAGTAGTCCCCAGTGTTCTTGTCACAGCTGGAG
ACTTTCAGCAGGGCAGTCATGCCCCTGTTCCTGAAGTCAGAGTTGTGGCAGCCCAGAATCCACAGGCC
AGGGTTCTCCATGCTCATGAACACAGTCTCCCCAGAGAAGGGGAACAGGGTCAGGGTGTCCTCATACA
CCATCTTGTGCTTGAAGGTGTAGCCAGAGAAGAACACAGACAGGAAGTCAGTCTGGGCCCCAATGCTC
AGGATGTACCAGTAGGCCACCTCATGCAGGCACACAGACAGCTGCAGGCTGTCAAACACATAGCCATT
GATGCTGTGCATGATGTTGCTGGCCTGGAACTCAGGGTCCTCCAGCTGCACCCCAGCAGGGTTGGGCA
GGAACCTCTGGATGTTCTCAGTCAGGTACCAGCTCCTGTTCTCATCAAACACAGAGAACAGGATCACA
TTCCTCTTGTCAGACATGATCTGGTTGCCCCTCTGGTCCACAGACTCCTTGTAGCAGATCAGCAGGGG
GCCAATCAGGCCAGAGGCCAGGTCCCTCTCCATGTTCACAAAGCTGCTGTAGTATCTGGTCAGGCACC
TGGGGTCAGACTTGGTGGGGCCATCCTCCACAGTCACAGTCCACTTGTACTTGAAGATCTCCCCAGGC
AGGATGGGGAAGTCCTTCAGGTGCTTCACCCCCTTGGGCAGCCTCCTGCTGTACAGGGGCCTCACATC
AGTGATGCCATGGGGGTAGATGTTGTAGGGCCTGCTGGCCTGGTTCTTGAAGATGATCAGCAGGGTGT
CCCCCACCTCCCCATACAGCAGGGGGCCCAGGATGCCAGACTCATGCTGGATGGCCTCCCTGGTCTTG
AAGGTTTCATCAGTGTAGGCCATGAACCTGACCTTCTTGTACTTCCTGCCAATCCTCTGGGGGCCATT
GTTCAGGTACTGGCTCTTGTAGCTCCTGTCATCAGGGGCCAGCACCAGGGGGGCATAGTCCCAGTCCT
CCTCCTCAGCAGCAATGTAGTGCACCCAGGTCTTGGGGTGCTTCTTGGCCACAGACCTGATCTGGATG
AAGCTGGGGCTGTTGTCATCATCAAACCTCACCACATCCATCTCAGAGTCAGTCAGGTCATCATCATA
GTCCTCAGCCTCCTCATTGTTCTTCATCCTCAGCTGGGGCTCCTCAGGGCAGCTGTCCACCTTCACAT
AGGCCTCCATGCCATCATGCTGGTGGCTGCTGATGTGGCAGAACAGCAGGAACTGGCCCAGGTCCATC
AGCAGGGTCTGGGCAGTCAGGAAGGTGATGGGGCTGATCTCCAGGCTGGCCTGCCTGTGGTTCCTGAC
CAGGAAGGTGTGGCCCTCCAGGAAGATGCTGTGCACCTCAGGGGTGGTGCCCATGCCAATCACATGCC
AGTACACAGACTTCCTGTGGCAGCCAATCAGGCCAGGCAGGCTCCTGTTCACATAGCCATTCACAGTG
TGCATCTTGGGCCAGGCCCTGGCAGAGGCAGCATCCCTGTCCTGCATCAGGCTGTTCTTGGTTTCAGA
GTGCCAGCTCTTGCCCTCATCAAACACAGCAAACAGCAGGATGAACTTGTGCAGGGTCTGGGTCTTCT
CCTTGGCCAGGCTGCCCTCCCTGCACACCAGCAGGGCCCCAATCAGGCCAGAGTTCAGGTCCTTCACC
AGGTCCACATGGCTCAGGTAGCTGTAGGTCAGGCACAGGGGGTCAGAGGCCATGGGGCCATTCTCCTT
CAGCACCTGCCACACATAGGTGTGGCTGCCCCCAGGGAACACCTTGTCATCCTCCTTCTCCCTCTGGC
TGGTCTGGTCATCATACTCAGCCCCCTCAGAGGCCTTCCAGTAGCTCACCCCCACAGCATGCAGGCTC
ACAGGGTGGCTGGCCATGTTCTTCAGGGTGATCACCACAGTGTCATACACCTCAGCCTGGATGGTGGG
GCCCAGCAGGCCCATCCAGGGGGGCCTGGGCTTGGCAATGTTGAACAGGTGGTCAGTGAACTCCACAA
ACAGGGTCTTCTTGTACACCACAGAGGTGTTGAAGGGGAAGCTCTTGGGCACTCTGGGGGGGAACCTG
GCATCCACAGGCAGCTCCCCCAGGTCAGACTGCATGTAGTCCCAGCTCAGCTCCACAGCCCCCAGGTA
GTATCTCCTGGTGGCAGAGAAGCAGAACCTCAGCAGGCACAGGAAGAAGCAGGTGCTCAGCTCAATCT
GCATGGTGGCGGCGTTTAAACCAACCTGAAAAAAAGTGATTTCAGGCAGGTGCTCCAGGTAATTAAAC
ATTAATACCCCACCAACCAACCATCCCTTAAACCCTTACCTCTTCAGGAGCTTGTGGATCTGTGTGAC
GGCTTCTCCTGGTGAAGGGGCTTTTATACCCCCTCCTTCCAACCCAGGCTGCTGATCCCTGCCAAGCT
GACTCCAAACCTGCTGATTCTGATTATTGACTTAGTCAACAAAAGGAGAATAAGTAACCTACACAAAT
ATGAACCTTGCCTAGGGAGATTAGAGTATCGGAACACTCGCTCTACGAAATGTGCAGACAGACGGGGA
TCCTCTAGAGCTACCTGCTGATCGCCCGGCCCCTGTTCAAACATGTCCTAATACTCTGTCTCTGCAAG
GGTCATCAGTAGTTTTCCATCTTACTCAACATCCTCCCAGTGGGTACCGTGGACTTAGCCCCTGTTTG
CTCCTCCGATAACTGGGGTGACCTTGGTTAATATTCACCAGCAGCCTCCCCCGGTGGACTTAGCCCCT
GTTTGCTCCTCCGATAACTGGGGTGACCTTGGTTAATATTCACCAGCAGCCTCCCCCGGTGGACTTAG
CCCCTGTTTGCTCCTCCGATAACTGGGGTGACCTTGGTTAATATTCACCAGCAGCCTCCCCCGCGGCC
GCGATAAGTAGCATGGCGGGTTAATCATTAACTACAAGGAACCCCTAGTGATGGAGCCTCAGCTTGGC
CACTCCCTCTCTGCGCGCTCGCTCGCTCACTGAGGCCGCCCGGGCAAAGCCCGGGCGTCGGGCGACCT
TTGGTCGCCCGGCCTCAIT
712 (Lanes 8-10; 6206 bp; hFVIII ORF 831-5222)
CTGAGGCCGCCCGGGCAAAGCCCGGGCGTCGGGCGACCTTTGGTCGCCCGGCCTCNGTGAGCGAGCGA
GCGCGCAGAGAGGGAGTGGCCAAGCTGAGGCTCCATCACTAGGGGTTCCTTGTAGTTAATGATTAACC
CGCCATGCTACTTATCGCGGCCGCGGGGGAGGCTGCTGGTGAATATTAACCAAGGTCACCCCAGTTAT
CGGAGGAGCAAACAGGGGCTAAGTCCACCGGGGGAGGCTGCTGGTGAATATTAACCAAGGTCACCCCA
GTTATCGGAGGAGCAAACAGGGGCTAAGTCCACCGGGGGAGGCTGCTGGTGAATATTAACCAAGGTCA
CCCCAGTTATCGGAGGAGCAAACAGGGGCTAAGTCCACGGTACCCACTGGGAGGATGTTGAGTAAGAT
GGAAAACTACTGATGACCCTTGCAGAGACAGAGTATTAGGACATGTTTGAACAGGGGCCGGGCGATCA
GCAGGTAGCTCTAGAGGATCCCCGTCTGTCTGCACATTTCGTAGAGCGAGTGTTCCGATACTCTAATC
TCCCTAGGCAAGGTTCATATTTGTGTAGGTTACTTATTCTCCTTTTGTTGACTAAGTCAATAATCAGA
ATCAGCAGGTTTGGAGTCAGCTTGGCAGGGATCAGCAGCCTGGGTTGGAAGGAGGGGGTATAAAAGCC
CCTTCACCAGGAGAAGCCGTCACACAGATCCACAAGCTCCTGAAGAGGTAAGGGTTTAAGGGATGGTT
GGTTGGTGGGGTATTAATGTTTAATTACCTGGAGCACCTGCCTGAAATCACTTTTTTTCAGGTTGGTT
TAAACGCCGCCACCATGCAGATTGAGCTGAGCACCTGCTTCTTCCTGTGCCTGCTGAGGTTCTGCTTC
TCTGCCACCAGGAGATACTACCTGGGGGCTGTGGAGCTGAGCTGGGACTACATGCAGTCTGACCTGGG
GGAGCTGCCTGTGGATGCCAGGTTCCCCCCCAGAGTGCCCAAGAGCTTCCCCTTCAACACCTCTGTGG
TGTACAAGAAGACCCTGTTTGTGGAGTTCACTGACCACCTGTTCAACATTGCCAAGCCCAGGCCCCCC
TGGATGGGCCTGCTGGGCCCCACCATCCAGGCTGAGGTGTATGACACTGTGGTGATCACCCTGAAGAA
CATGGCCAGCCACCCTGTGAGCCTGCATGCTGTGGGGGTGAGCTACTGGAAGGCCTCTGAGGGGGCTG
AGTATGATGACCAGACCAGCCAGAGGGAGAAGGAGGATGACAAGGTGTTCCCTGGGGGCAGCCACACC
TATGTGTGGCAGGTGCTGAAGGAGAATGGCCCCATGGCCTCTGACCCCCTGTGCCTGACCTACAGCTA
CCTGAGCCATGTGGACCTGGTGAAGGACCTGAACTCTGGCCTGATTGGGGCCCTGCTGGTGTGCAGGG
AGGGCAGCCTGGCCAAGGAGAAGACCCAGACCCTGCACAAGTTCATCCTGCTGTTTGCTGTGTTTGAT
GAGGGCAAGAGCTGGCACTCTGAAACCAAGAACAGCCTGATGCAGGACAGGGATGCTGCCTCTGCCAG
GGCCTGGCCCAAGATGCACACTGTGAATGGCTATGTGAACAGGAGCCTGCCTGGCCTGATTGGCTGCC
ACAGGAAGTCTGTGTACTGGCATGTGATTGGCATGGGCACCACCCCTGAGGTGCACAGCATCTTCCTG
GAGGGCCACACCTTCCTGGTCAGGAACCACAGGCAGGCCAGCCTGGAGATCAGCCCCATCACCTTCCT
GACTGCCCAGACCCTGCTGATGGACCTGGGCCAGTTCCTGCTGTTCTGCCACATCAGCAGCCACCAGC
ATGATGGCATGGAGGCCTATGTGAAGGTGGACAGCTGCCCTGAGGAGCCCCAGCTGAGGATGAAGAAC
AATGAGGAGGCTGAGGACTATGATGATGACCTGACTGACTCTGAGATGGATGTGGTGAGGTTTGATGA
TGACAACAGCCCCAGCTTCATCCAGATCAGGTCTGTGGCCAAGAAGCACCCCAAGACCTGGGTGCACT
ACATTGCTGCTGAGGAGGAGGACTGGGACTATGCCCCCCTGGTGCTGGCCCCTGATGACAGGAGCTAC
AAGAGCCAGTACCTGAACAATGGCCCCCAGAGGATTGGCAGGAAGTACAAGAAGGTCAGGTTCATGGC
CTACACTGATGAAACCTTCAAGACCAGGGAGGCCATCCAGCATGAGTCTGGCATCCTGGGCCCCCTGC
TGTATGGGGAGGTGGGGGACACCCTGCTGATCATCTTCAAGAACCAGGCCAGCAGGCCCTACAACATC
TACCCCCATGGCATCACTGATGTGAGGCCCCTGTACAGCAGGAGGCTGCCCAAGGGGGTGAAGCACCT
GAAGGACTTCCCCATCCTGCCTGGGGAGATCTTCAAGTACAAGTGGACTGTGACTGTGGAGGATGGCC
CCACCAAGTCTGACCCCAGGTGCCTGACCAGATACTACAGCAGCTTTGTGAACATGGAGAGGGACCTG
GCCTCTGGCCTGATTGGCCCCCTGCTGATCTGCTACAAGGAGTCTGTGGACCAGAGGGGCAACCAGAT
CATGTCTGACAAGAGGAATGTGATCCTGTTCTCTGTGTTTGATGAGAACAGGAGCTGGTACCTGACTG
AGAACATCCAGAGGTTCCTGCCCAACCCTGCTGGGGTGCAGCTGGAGGACCCTGAGTTCCAGGCCAGC
AACATCATGCACAGCATCAATGGCTATGTGTTTGACAGCCTGCAGCTGTCTGTGTGCCTGCATGAGGT
GGCCTACTGGTACATCCTGAGCATTGGGGCCCAGACTGACTTCCTGTCTGTGTTCTTCTCTGGCTACA
CCTTCAAGCACAAGATGGTGTATGAGGACACCCTGACCCTGTTCCCCTTCTCTGGGGAGACTGTGTTC
ATGAGCATGGAGAACCCTGGCCTGTGGATTCTGGGCTGCCACAACTCTGACTTCAGGAACAGGGGCAT
GACTGCCCTGCTGAAAGTCTCCAGCTGTGACAAGAACACTGGGGACTACTATGAGGACAGCTATGAGG
ACATCTCTGCCTACCTGCTGAGCAAGAACAATGCCATTGAGCCCAGGAGCTTCAGCCAGAATAGCAGG
CACCCCAGCACCAGGCAGAAGCAGTTCAATGCCACCACCATCCCAGAGAATACCACCCTGCAGTCTGA
CCAGGAGGAGATTGACTATGATGACACCATCTCTGTGGAGATGAAGAAGGAGGACTTTGACATCTACG
ACGAGGACGAGAACCAGAGCCCCAGGAGCTTCCAGAAGAAGACCAGGCACTACTTCATTGCTGCTGTG
GAGAGGCTGTGGGACTATGGCATGAGCAGCAGCCCCCATGTGCTGAGGAACAGGGCCCAGTCTGGCTC
TGTGCCCCAGTTCAAGAAGGTGGTGTTCCAGGAGTTCACTGATGGCAGCTTCACCCAGCCCCTGTACA
GAGGGGAGCTGAATGAGCACCTGGGCCTGCTGGGCCCCTACATCAGGGCTGAGGTGGAGGACAACATC
ATGGTGACCTTCAGGAACCAGGCCAGCAGGCCCTACAGCTTCTACAGCAGCCTGATCAGCTATGAGGA
GGACCAGAGGCAGGGGGCTGAGCCCAGGAAGAACTTTGTGAAGCCCAATGAAACCAAGACCTACTTCT
GGAAGGTGCAGCACCACATGGCCCCCACCAAGGATGAGTTTGACTGCAAGGCCTGGGCCTACTTCTCT
GATGTGGACCTGGAGAAGGATGTGCACTCTGGCCTGATTGGCCCCCTGCTGGTGTGCCACACCAACAC
CCTGAACCCTGCCCATGGCAGGCAGGTGACTGTGCAGGAGTTTGCCCTGTTCTTCACCATCTTTGATG
AAACCAAGAGCTGGTACTTCACTGAGAACATGGAGAGGAACTGCAGGGCCCCCTGCAACATCCAGATG
GAGGACCCCACCTTCAAGGAGAACTACAGGTTCCATGCCATCAATGGCTACATCATGGACACCCTGCC
TGGCCTGGTGATGGCCCAGGACCAGAGGATCAGGTGGTACCTGCTGAGCATGGGCAGCAATGAGAACA
TCCACAGCATCCACTTCTCTGGCCATGTGTTCACTGTGAGGAAGAAGGAGGAGTACAAGATGGCCCTG
TACAACCTGTACCCTGGGGTGTTTGAGACTGTGGAGATGCTGCCCAGCAAGGCTGGCATCTGGAGGGT
GGAGTGCCTGATTGGGGAGCACCTGCATGCTGGCATGAGCACCCTGTTCCTGGTGTACAGCAACAAGT
GCCAGACCCCCCTGGGCATGGCCTCTGGCCACATCAGGGACTTCCAGATCACTGCCTCTGGCCAGTAT
GGCCAGTGGGCCCCCAAGCTGGCCAGGCTGCACTACTCTGGCAGCATCAATGCCTGGAGCACCAAGGA
GCCCTTCAGCTGGATCAAGGTGGACCTGCTGGCCCCCATGATCATCCATGGCATCAAGACCCAGGGGG
CCAGGCAGAAGTTCAGCAGCCTGTACATCAGCCAGTTCATCATCATGTACAGCCTGGATGGCAAGAAG
TGGCAGACCTACAGGGGCAACAGCACTGGCACCCTGATGGTGTTCTTTGGCAATGTGGACAGCTCTGG
CATCAAGCACAACATCTTCAACCCCCCCATCATTGCCAGATACATCAGGCTGCACCCCACCCACTACA
GCATCAGGAGCACCCTGAGGATGGAGCTGATGGGCTGTGACCTGAACAGCTGCAGCATGCCCCTGGGC
ATGGAGAGCAAGGCCATCTCTGATGCCCAGATCACTGCCAGCAGCTACTTCACCAACATGTTTGCCAC
CTGGAGCCCCAGCAAGGCCAGGCTGCACCTGCAGGGCAGGAGCAATGCCTGGAGGCCCCAGGTCAACA
ACCCCAAGGAGTGGCTGCAGGTGGACTTCCAGAAGACCATGAAGGTGACTGGGGTGACCACCCAGGGG
GTGAAGAGCCTGCTGACCAGCATGTATGTGAAGGAGTTCCTGATCAGCAGCAGCCAGGATGGCCACCA
GTGGACCCTGTTCTTCCAGAATGGCAAGGTGAAGGTGTTCCAGGGCAACCAGGACAGCTTCACCCCTG
TGGTGAACAGCCTGGACCCCCCCCTGCTGACCAGATACCTGAGGATTCACCCCCAGAGCTGGGTGCAC
CAGATTGCCCTGAGGATGGAGGTGCTGGGCTGTGAGGCCCAGGACCTGTACTGATTAATTAAGAGCAT
CTTACCGCCATTTATTCCCATATTTGTTCTGTTTTTCTTGATTTGGGTATACATTTAAATGTTAATAA
AACAAAATGGTGGGGCAATCATTTACATTTTTAGGGATATGTAATTACTAGTTCAGGTGTATTGCCAC
AAGACAAACATGTTAAGAAACTTTCCCGTTATTTACGCTCTGTTCCTGTTAATCAACCTCTGGATTAC
AAAATTTGTGAAAGATTGACTGATATTCTTAACTATGTTGCTCCTTTTACGCTGTGTGGATATGCTGC
TTTATAGCCTCTGTATCTAGCTATTGCTTCCCGTACGGCTTTCGTTTTCTCCTCCTTGTATAAATCCT
GGTTGCTGTCTCTTTTAGAGGAGTTGTGGCCCGTTGTCCGTCAACGTGGCGTGGTGTGCTCTGTGTTT
GCTGACGCAACCCCCACTGGCTGGGGCATTGCCACCACCTGTCAACTCCTTTCTGGGACTTTCGCTTT
CCCCCTCCCGATCGCCACGGCAGAACTCATCGCCGCCTGCCTTGCCCGCTGCTGGACAGGGGCTAGGT
TGCTGGGCACTGATAATTCCGTGGTGTTGTCTGTGCCTTCTAGTTGCCAGCCATCTGTTGTTTGCCCC
TCCCCCGTGCCTTCCTTGACCCTGGAAGGTGCCACTCCCACTGTCCTTTCCTAATAAAATGAGGAAAT
TGCATCGCATTGTCTGAGTAGGTGTCATTCTATTCTGGGGGGTGGGGTGGGGCAGGACAGCAAGGGGG
AGGATTGGGAAGACAATAGCAGGCATGCTGGGGATGCGGTGGGCTCTATGGCTCTAGAGCATGGCTAC
GTAGATAAGTAGCATGGCGGGTTAATCATTAACTACACCTGCAGGAGGAACCCCTAGTGATGGAGTTG
GCCACTCCCTCTCTGCGCGCTCGCTCGCTCACTGAGGCCGCCCGGGCAAAGCCCGGGCGTCGGGCGAC
CTTTGGTCGCCCGGCCTC
086 (Lanes 9-10; 6206 bp; hFVIII ORF 985-5376)
GAGGCCGCCCGGGCAAAGCCCGGGCGTCGGGCGACCTTTGGTCGCCCGGCCTCAGTGAGCGAGCGAGC
GCGCAGAGAGGGAGTGGCCAACTCCATCACTAGGGGTTCCTCCTGCAGGTGTAGTTAATGATTAACCC
GCCATGCTACTTATCTACGTAGCCATGCTCTAGAGCCATAGAGCCCACCGCATCCCCAGCATGCCTGC
TATTGTCTTCCCAATCCTCCCCCTTGCTGTCCTGCCCCACCCCACCCCCCAGAATAGAATGACACCTA
CTCAGACAATGCGATGCAATTTCCTCATTTTATTAGGAAAGGACAGTGGGAGTGGCACCTTCCAGGGT
CAAGGAAGGCACGGGGGAGGGGCAAACAACAGATGGCTGGCAACTAGAAGGCACAGACAACACCACGG
AATTATCAGTGCCCAGCAACCTAGCCCCTGTCCAGCAGCGGGCAAGGCAGGCGGCGATGAGTTCTGCC
GTGGCGATCGGGAGGGGGAAAGCGAAAGTCCCAGAAAGGAGTTGACAGGTGGTGGCAATGCCCCAGCC
AGTGGGGGTTGCGTCAGCAAACACAGAGCACACCACGCCACGTTGACGGACAACGGGCCACAACTCCT
CTAAAAGAGACAGCAACCAGGATTTATACAAGGAGGAGAAAACGAAAGCCGTACGGGAAGCAATAGCT
AGATACAGAGGCTATAAAGCAGCATATCCACACAGCGTAAAAGGAGCAACATAGTTAAGAATATCAGT
CAATCTTTCACAAATTTTGTAATCCAGAGGTTGATTAACAGGAACAGAGCGTAAATAACGGGAAAGTT
TCTTAACATGTTTGTCTTGTGGCAATACACCTGAACTAGTAATTACATATCCCTAAAAATGTAAATGA
TTGCCCCACCATTTTGTTTTATTAACATTTAAATGTATACCCAAATCAAGAAAAACAGAACAAATATG
GGAATAAATGGCGGTAAGATGCTCTTAATTAATCAGTACAGGTCCTGGGCCTCACAGCCCAGCACCTC
CATCCTCAGGGCAATCTGGTGCACCCAGCTCTGGGGGTGAATCCTCAGGTATCTGGTCAGCAGGGGGG
GGTCCAGGCTGTTCACCACAGGGGTGAAGCTGTCCTGGTTGCCCTGGAACACCTTCACCTTGCCATTC
TGGAAGAACAGGGTCCACTGGTGGCCATCCTGGCTGCTGCTGATCAGGAACTCCTTCACATACATGCT
GGTCAGCAGGCTCTTCACCCCCTGGGTGGTCACCCCAGTCACCTTCATGGTCTTCTGGAAGTCCACCT
GCAGCCACTCCTTGGGGTTGTTGACCTGGGGCCTCCAGGCATTGCTCCTGCCCTGCAGGTGCAGCCTG
GCCTTGCTGGGGCTCCAGGTGGCAAACATGTTGGTGAAGTAGCTGCTGGCAGTGATCTGGGCATCAGA
GATGGCCTTGCTCTCCATGCCCAGGGGCATGCTGCAGCTGTTCAGGTCACAGCCCATCAGCTCCATCC
TCAGGGTGCTCCTGATGCTGTAGTGGGTGGGGTGCAGCCTGATGTATCTGGCAATGATGGGGGGGTTG
AAGATGTTGTGCTTGATGCCAGAGCTGTCCACATTGCCAAAGAACACCATCAGGGTGCCAGTGCTGTT
GCCCCTGTAGGTCTGCCACTTCTTGCCATCCAGGCTGTACATGATGATGAACTGGCTGATGTACAGGC
TGCTGAACTTCTGCCTGGCCCCCTGGGTCTTGATGCCATGGATGATCATGGGGGCCAGCAGGTCCACC
TTGATCCAGCTGAAGGGCTCCTTGGTGCTCCAGGCATTGATGCTGCCAGAGTAGTGCAGCCTGGCCAG
CTTGGGGGCCCACTGGCCATACTGGCCAGAGGCAGTGATCTGGAAGTCCCTGATGTGGCCAGAGGCCA
TGCCCAGGGGGGTCTGGCACTTGTTGCTGTACACCAGGAACAGGGTGCTCATGCCAGCATGCAGGTGC
TCCCCAATCAGGCACTCCACCCTCCAGATGCCAGCCTTGCTGGGCAGCATCTCCACAGTCTCAAACAC
CCCAGGGTACAGGTTGTACAGGGCCATCTTGTACTCCTCCTTCTTCCTCACAGTGAACACATGGCCAG
AGAAGTGGATGCTGTGGATGTTCTCATTGCTGCCCATGCTCAGCAGGTACCACCTGATCCTCTGGTCC
TGGGCCATCACCAGGCCAGGCAGGGTGTCCATGATGTAGCCATTGATGGCATGGAACCTGTAGTTCTC
CTTGAAGGTGGGGTCCTCCATCTGGATGTTGCAGGGGGCCCTGCAGTTCCTCTCCATGTTCTCAGTGA
AGTACCAGCTCTTGGTTTCATCAAAGATGGTGAAGAACAGGGCAAACTCCTGCACAGTCACCTGCCTG
CCATGGGCAGGGTTCAGGGTGTTGGTGTGGCACACCAGCAGGGGGCCAATCAGGCCAGAGTGCACATC
CTTCTCCAGGTCCACATCAGAGAAGTAGGCCCAGGCCTTGCAGTCAAACTCATCCTTGGTGGGGGCCA
TGTGGTGCTGCACCTTCCAGAAGTAGGTCTTGGTTTCATTGGGCTTCACAAAGTTCTTCCTGGGCTCA
GCCCCCTGCCTCTGGTCCTCCTCATAGCTGATCAGGCTGCTGTAGAAGCTGTAGGGCCTGCTGGCCTG
GTTCCTGAAGGTCACCATGATGTTGTCCTCCACCTCAGCCCTGATGTAGGGGCCCAGCAGGCCCAGGT
GCTCATTCAGCTCCCCTCTGTACAGGGGCTGGGTGAAGCTGCCATCAGTGAACTCCTGGAACACCACC
TTCTTGAACTGGGGCACAGAGCCAGACTGGGCCCTGTTCCTCAGCACATGGGGGCTGCTGCTCATGCC
ATAGTCCCACAGCCTCTCCACAGCAGCAATGAAGTAGTGCCTGGTCTTCTTCTGGAAGCTCCTGGGGC
TCTGGTTCTCGTCCTCGTCGTAGATGTCAAAGTCCTCCTTCTTCATCTCCACAGAGATGGTGTCATCA
TAGTCAATCTCCTCCTGGTCAGACTGCAGGGTGGTATTCTCTGGGATGGTGGTGGCATTGAACTGCTT
CTGCCTGGTGCTGGGGTGCCTGCTATTCTGGCTGAAGCTCCTGGGCTCAATGGCATTGTTCTTGCTCA
GCAGGTAGGCAGAGATGTCCTCATAGCTGTCCTCATAGTAGTCCCCAGTGTTCTTGTCACAGCTGGAG
ACTTTCAGCAGGGCAGTCATGCCCCTGTTCCTGAAGTCAGAGTTGTGGCAGCCCAGAATCCACAGGCC
AGGGTTCTCCATGCTCATGAACACAGTCTCCCCAGAGAAGGGGAACAGGGTCAGGGTGTCCTCATACA
CCATCTTGTGCTTGAAGGTGTAGCCAGAGAAGAACACAGACAGGAAGTCAGTCTGGGCCCCAATGCTC
AGGATGTACCAGTAGGCCACCTCATGCAGGCACACAGACAGCTGCAGGCTGTCAAACACATAGCCATT
GATGCTGTGCATGATGTTGCTGGCCTGGAACTCAGGGTCCTCCAGCTGCACCCCAGCAGGGTTGGGCA
GGAACCTCTGGATGTTCTCAGTCAGGTACCAGCTCCTGTTCTCATCAAACACAGAGAACAGGATCACA
TTCCTCTTGTCAGACATGATCTGGTTGCCCCTCTGGTCCACAGACTCCTTGTAGCAGATCAGCAGGGG
GCCAATCAGGCCAGAGGCCAGGTCCCTCTCCATGTTCACAAAGCTGCTGTAGTATCTGGTCAGGCACC
TGGGGTCAGACTTGGTGGGGCCATCCTCCACAGTCACAGTCCACTTGTACTTGAAGATCTCCCCAGGC
AGGATGGGGAAGTCCTTCAGGTGCTTCACCCCCTTGGGCAGCCTCCTGCTGTACAGGGGCCTCACATC
AGTGATGCCATGGGGGTAGATGTTGTAGGGCCTGCTGGCCTGGTTCTTGAAGATGATCAGCAGGGTGT
CCCCCACCTCCCCATACAGCAGGGGGCCCAGGATGCCAGACTCATGCTGGATGGCCTCCCTGGTCTTG
AAGGTTTCATCAGTGTAGGCCATGAACCTGACCTTCTTGTACTTCCTGCCAATCCTCTGGGGGCCATT
GTTCAGGTACTGGCTCTTGTAGCTCCTGTCATCAGGGGCCAGCACCAGGGGGGCATAGTCCCAGTCCT
CCTCCTCAGCAGCAATGTAGTGCACCCAGGTCTTGGGGTGCTTCTTGGCCACAGACCTGATCTGGATG
AAGCTGGGGCTGTTGTCATCATCAAACCTCACCACATCCATCTCAGAGTCAGTCAGGTCATCATCATA
GTCCTCAGCCTCCTCATTGTTCTTCATCCTCAGCTGGGGCTCCTCAGGGCAGCTGTCCACCTTCACAT
AGGCCTCCATGCCATCATGCTGGTGGCTGCTGATGTGGCAGAACAGCAGGAACTGGCCCAGGTCCATC
AGCAGGGTCTGGGCAGTCAGGAAGGTGATGGGGCTGATCTCCAGGCTGGCCTGCCTGTGGTTCCTGAC
CAGGAAGGTGTGGCCCTCCAGGAAGATGCTGTGCACCTCAGGGGTGGTGCCCATGCCAATCACATGCC
AGTACACAGACTTCCTGTGGCAGCCAATCAGGCCAGGCAGGCTCCTGTTCACATAGCCATTCACAGTG
TGCATCTTGGGCCAGGCCCTGGCAGAGGCAGCATCCCTGTCCTGCATCAGGCTGTTCTTGGTTTCAGA
GTGCCAGCTCTTGCCCTCATCAAACACAGCAAACAGCAGGATGAACTTGTGCAGGGTCTGGGTCTTCT
CCTTGGCCAGGCTGCCCTCCCTGCACACCAGCAGGGCCCCAATCAGGCCAGAGTTCAGGTCCTTCACC
AGGTCCACATGGCTCAGGTAGCTGTAGGTCAGGCACAGGGGGTCAGAGGCCATGGGGCCATTCTCCTT
CAGCACCTGCCACACATAGGTGTGGCTGCCCCCAGGGAACACCTTGTCATCCTCCTTCTCCCTCTGGC
TGGTCTGGTCATCATACTCAGCCCCCTCAGAGGCCTTCCAGTAGCTCACCCCCACAGCATGCAGGCTC
ACAGGGTGGCTGGCCATGTTCTTCAGGGTGATCACCACAGTGTCATACACCTCAGCCTGGATGGTGGG
GCCCAGCAGGCCCATCCAGGGGGGCCTGGGCTTGGCAATGTTGAACAGGTGGTCAGTGAACTCCACAA
ACAGGGTCTTCTTGTACACCACAGAGGTGTTGAAGGGGAAGCTCTTGGGCACTCTGGGGGGGAACCTG
GCATCCACAGGCAGCTCCCCCAGGTCAGACTGCATGTAGTCCCAGCTCAGCTCCACAGCCCCCAGGTA
GTATCTCCTGGTGGCAGAGAAGCAGAACCTCAGCAGGCACAGGAAGAAGCAGGTGCTCAGCTCAATCT
GCATGGTGGCGGCGTTTAAACCAACCTGAAAAAAAGTGATTTCAGGCAGGTGCTCCAGGTAATTAAAC
ATTAATACCCCACCAACCAACCATCCCTTAAACCCTTACCTCTTCAGGAGCTTGTGGATCTGTGTGAC
GGCTTCTCCTGGTGAAGGGGCTTTTATACCCCCTCCTTCCAACCCAGGCTGCTGATCCCTGCCAAGCT
GACTCCAAACCTGCTGATTCTGATTATTGACTTAGTCAACAAAAGGAGAATAAGTAACCTACACAAAT
ATGAACCTTGCCTAGGGAGATTAGAGTATCGGAACACTCGCTCTACGAAATGTGCAGACAGACGGGGA
TCCTCTAGAGCTACCTGCTGATCGCCCGGCCCCTGTTCAAACATGTCCTAATACTCTGTCTCTGCAAG
GGTCATCAGTAGTTTTCCATCTTACTCAACATCCTCCCAGTGGGTACCGTGGACTTAGCCCCTGTTTG
CTCCTCCGATAACTGGGGTGACCTTGGTTAATATTCACCAGCAGCCTCCCCCGGTGGACTTAGCCCCT
GTTTGCTCCTCCGATAACTGGGGTGACCTTGGTTAATATTCACCAGCAGCCTCCCCCGGTGGACTTAG
CCCCTGTTTGCTCCTCCGATAACTGGGGTGACCTTGGTTAATATTCACCAGCAGCCTCCCCCGCGGCC
GCGATAAGTAGCATGGCGGGTTAATCATTAACTACAAGGAACCCCTAGTGATGGAGCCTCAGCTTGGC
CACTCCCTCTCTGCGCGCTCGCTCGCTCACTGAGGCCGCCCGGGCAAAGCCCGGGCGTCGGGCGACCT
TTGGTCGCCCGGCCTCIG
713 (Lanes 11-13; 6206 bp; hFVIII ORF 831-5222)
CTGAGGCCGCCCGGGCAAAGCCCGGGCGTCGGGCGACCTTTGGTCGCCCGGCNTCAGTGAGCGAGCGA
GCGCGCAGAGAGGGAGTGGCCAAGCTGAGGCTCCATCACTAGGGGTTCCTTGTAGTTAATGATTAACC
CGCCATGCTACTTATCGCGGCCGCGGGGGAGGCTGCTGGTGAATATTAACCAAGGTCACCCCAGTTAT
CGGAGGAGCAAACAGGGGCTAAGTCCACCGGGGGAGGCTGCTGGTGAATATTAACCAAGGTCACCCCA
GTTATCGGAGGAGCAAACAGGGGCTAAGTCCACCGGGGGAGGCTGCTGGTGAATATTAACCAAGGTCA
CCCCAGTTATCGGAGGAGCAAACAGGGGCTAAGTCCACGGTACCCACTGGGAGGATGTTGAGTAAGAT
GGAAAACTACTGATGACCCTTGCAGAGACAGAGTATTAGGACATGTTTGAACAGGGGCCGGGCGATCA
GCAGGTAGCTCTAGAGGATCCCCGTCTGTCTGCACATTTCGTAGAGCGAGTGTTCCGATACTCTAATC
TCCCTAGGCAAGGTTCATATTTGTGTAGGTTACTTATTCTCCTTTTGTTGACTAAGTCAATAATCAGA
ATCAGCAGGTTTGGAGTCAGCTTGGCAGGGATCAGCAGCCTGGGTTGGAAGGAGGGGGTATAAAAGCC
CCTTCACCAGGAGAAGCCGTCACACAGATCCACAAGCTCCTGAAGAGGTAAGGGTTTAAGGGATGGTT
GGTTGGTGGGGTATTAATGTTTAATTACCTGGAGCACCTGCCTGAAATCACTTTTTTTCAGGTTGGTT
TAAACGCCGCCACCATGCAGATTGAGCTGAGCACCTGCTTCTTCCTGTGCCTGCTGAGGTTCTGCTTC
TCTGCCACCAGGAGATACTACCTGGGGGCTGTGGAGCTGAGCTGGGACTACATGCAGTCTGACCTGGG
GGAGCTGCCTGTGGATGCCAGGTTCCCCCCCAGAGTGCCCAAGAGCTTCCCCTTCAACACCTCTGTGG
TGTACAAGAAGACCCTGTTTGTGGAGTTCACTGACCACCTGTTCAACATTGCCAAGCCCAGGCCCCCC
TGGATGGGCCTGCTGGGCCCCACCATCCAGGCTGAGGTGTATGACACTGTGGTGATCACCCTGAAGAA
CATGGCCAGCCACCCTGTGAGCCTGCATGCTGTGGGGGTGAGCTACTGGAAGGCCTCTGAGGGGGCTG
AGTATGATGACCAGACCAGCCAGAGGGAGAAGGAGGATGACAAGGTGTTCCCTGGGGGCAGCCACACC
TATGTGTGGCAGGTGCTGAAGGAGAATGGCCCCATGGCCTCTGACCCCCTGTGCCTGACCTACAGCTA
CCTGAGCCATGTGGACCTGGTGAAGGACCTGAACTCTGGCCTGATTGGGGCCCTGCTGGTGTGCAGGG
AGGGCAGCCTGGCCAAGGAGAAGACCCAGACCCTGCACAAGTTCATCCTGCTGTTTGCTGTGTTTGAT
GAGGGCAAGAGCTGGCACTCTGAAACCAAGAACAGCCTGATGCAGGACAGGGATGCTGCCTCTGCCAG
GGCCTGGCCCAAGATGCACACTGTGAATGGCTATGTGAACAGGAGCCTGCCTGGCCTGATTGGCTGCC
ACAGGAAGTCTGTGTACTGGCATGTGATTGGCATGGGCACCACCCCTGAGGTGCACAGCATCTTCCTG
GAGGGCCACACCTTCCTGGTCAGGAACCACAGGCAGGCCAGCCTGGAGATCAGCCCCATCACCTTCCT
GACTGCCCAGACCCTGCTGATGGACCTGGGCCAGTTCCTGCTGTTCTGCCACATCAGCAGCCACCAGC
ATGATGGCATGGAGGCCTATGTGAAGGTGGACAGCTGCCCTGAGGAGCCCCAGCTGAGGATGAAGAAC
AATGAGGAGGCTGAGGACTATGATGATGACCTGACTGACTCTGAGATGGATGTGGTGAGGTTTGATGA
TGACAACAGCCCCAGCTTCATCCAGATCAGGTCTGTGGCCAAGAAGCACCCCAAGACCTGGGTGCACT
ACATTGCTGCTGAGGAGGAGGACTGGGACTATGCCCCCCTGGTGCTGGCCCCTGATGACAGGAGCTAC
AAGAGCCAGTACCTGAACAATGGCCCCCAGAGGATTGGCAGGAAGTACAAGAAGGTCAGGTTCATGGC
CTACACTGATGAAACCTTCAAGACCAGGGAGGCCATCCAGCATGAGTCTGGCATCCTGGGCCCCCTGC
TGTATGGGGAGGTGGGGGACACCCTGCTGATCATCTTCAAGAACCAGGCCAGCAGGCCCTACAACATC
TACCCCCATGGCATCACTGATGTGAGGCCCCTGTACAGCAGGAGGCTGCCCAAGGGGGTGAAGCACCT
GAAGGACTTCCCCATCCTGCCTGGGGAGATCTTCAAGTACAAGTGGACTGTGACTGTGGAGGATGGCC
CCACCAAGTCTGACCCCAGGTGCCTGACCAGATACTACAGCAGCTTTGTGAACATGGAGAGGGACCTG
GCCTCTGGCCTGATTGGCCCCCTGCTGATCTGCTACAAGGAGTCTGTGGACCAGAGGGGCAACCAGAT
CATGTCTGACAAGAGGAATGTGATCCTGTTCTCTGTGTTTGATGAGAACAGGAGCTGGTACCTGACTG
AGAACATCCAGAGGTTCCTGCCCAACCCTGCTGGGGTGCAGCTGGAGGACCCTGAGTTCCAGGCCAGC
AACATCATGCACAGCATCAATGGCTATGTGTTTGACAGCCTGCAGCTGTCTGTGTGCCTGCATGAGGT
GGCCTACTGGTACATCCTGAGCATTGGGGCCCAGACTGACTTCCTGTCTGTGTTCTTCTCTGGCTACA
CCTTCAAGCACAAGATGGTGTATGAGGACACCCTGACCCTGTTCCCCTTCTCTGGGGAGACTGTGTTC
ATGAGCATGGAGAACCCTGGCCTGTGGATTCTGGGCTGCCACAACTCTGACTTCAGGAACAGGGGCAT
GACTGCCCTGCTGAAAGTCTCCAGCTGTGACAAGAACACTGGGGACTACTATGAGGACAGCTATGAGG
ACATCTCTGCCTACCTGCTGAGCAAGAACAATGCCATTGAGCCCAGGAGCTTCAGCCAGAATAGCAGG
CACCCCAGCACCAGGCAGAAGCAGTTCAATGCCACCACCATCCCAGAGAATACCACCCTGCAGTCTGA
CCAGGAGGAGATTGACTATGATGACACCATCTCTGTGGAGATGAAGAAGGAGGACTTTGACATCTACG
ACGAGGACGAGAACCAGAGCCCCAGGAGCTTCCAGAAGAAGACCAGGCACTACTTCATTGCTGCTGTG
GAGAGGCTGTGGGACTATGGCATGAGCAGCAGCCCCCATGTGCTGAGGAACAGGGCCCAGTCTGGCTC
TGTGCCCCAGTTCAAGAAGGTGGTGTTCCAGGAGTTCACTGATGGCAGCTTCACCCAGCCCCTGTACA
GAGGGGAGCTGAATGAGCACCTGGGCCTGCTGGGCCCCTACATCAGGGCTGAGGTGGAGGACAACATC
ATGGTGACCTTCAGGAACCAGGCCAGCAGGCCCTACAGCTTCTACAGCAGCCTGATCAGCTATGAGGA
GGACCAGAGGCAGGGGGCTGAGCCCAGGAAGAACTTTGTGAAGCCCAATGAAACCAAGACCTACTTCT
GGAAGGTGCAGCACCACATGGCCCCCACCAAGGATGAGTTTGACTGCAAGGCCTGGGCCTACTTCTCT
GATGTGGACCTGGAGAAGGATGTGCACTCTGGCCTGATTGGCCCCCTGCTGGTGTGCCACACCAACAC
CCTGAACCCTGCCCATGGCAGGCAGGTGACTGTGCAGGAGTTTGCCCTGTTCTTCACCATCTTTGATG
AAACCAAGAGCTGGTACTTCACTGAGAACATGGAGAGGAACTGCAGGGCCCCCTGCAACATCCAGATG
GAGGACCCCACCTTCAAGGAGAACTACAGGTTCCATGCCATCAATGGCTACATCATGGACACCCTGCC
TGGCCTGGTGATGGCCCAGGACCAGAGGATCAGGTGGTACCTGCTGAGCATGGGCAGCAATGAGAACA
TCCACAGCATCCACTTCTCTGGCCATGTGTTCACTGTGAGGAAGAAGGAGGAGTACAAGATGGCCCTG
TACAACCTGTACCCTGGGGTGTTTGAGACTGTGGAGATGCTGCCCAGCAAGGCTGGCATCTGGAGGGT
GGAGTGCCTGATTGGGGAGCACCTGCATGCTGGCATGAGCACCCTGTTCCTGGTGTACAGCAACAAGT
GCCAGACCCCCCTGGGCATGGCCTCTGGCCACATCAGGGACTTCCAGATCACTGCCTCTGGCCAGTAT
GGCCAGTGGGCCCCCAAGCTGGCCAGGCTGCACTACTCTGGCAGCATCAATGCCTGGAGCACCAAGGA
GCCCTTCAGCTGGATCAAGGTGGACCTGCTGGCCCCCATGATCATCCATGGCATCAAGACCCAGGGGG
CCAGGCAGAAGTTCAGCAGCCTGTACATCAGCCAGTTCATCATCATGTACAGCCTGGATGGCAAGAAG
TGGCAGACCTACAGGGGCAACAGCACTGGCACCCTGATGGTGTTCTTTGGCAATGTGGACAGCTCTGG
CATCAAGCACAACATCTTCAACCCCCCCATCATTGCCAGATACATCAGGCTGCACCCCACCCACTACA
GCATCAGGAGCACCCTGAGGATGGAGCTGATGGGCTGTGACCTGAACAGCTGCAGCATGCCCCTGGGC
ATGGAGAGCAAGGCCATCTCTGATGCCCAGATCACTGCCAGCAGCTACTTCACCAACATGTTTGCCAC
CTGGAGCCCCAGCAAGGCCAGGCTGCACCTGCAGGGCAGGAGCAATGCCTGGAGGCCCCAGGTCAACA
ACCCCAAGGAGTGGCTGCAGGTGGACTTCCAGAAGACCATGAAGGTGACTGGGGTGACCACCCAGGGG
GTGAAGAGCCTGCTGACCAGCATGTATGTGAAGGAGTTCCTGATCAGCAGCAGCCAGGATGGCCACCA
GTGGACCCTGTTCTTCCAGAATGGCAAGGTGAAGGTGTTCCAGGGCAACCAGGACAGCTTCACCCCTG
TGGTGAACAGCCTGGACCCCCCCCTGCTGACCAGATACCTGAGGATTCACCCCCAGAGCTGGGTGCAC
CAGATTGCCCTGAGGATGGAGGTGCTGGGCTGTGAGGCCCAGGACCTGTACTGATTAATTAAGAGCAT
CTTACCGCCATTTATTCCCATATTTGTTCTGTTTTTCTTGATTTGGGTATACATTTAAATGTTAATAA
AACAAAATGGTGGGGCAATCATTTACATTTTTAGGGATATGTAATTACTAGTTCAGGTGTATTGCCAC
AAGACAAACATGTTAAGAAACTTTCCCGTTATTTACGCTCTGTTCCTGTTAATCAACCTCTGGATTAC
AAAATTTGTGAAAGATTGACTGATATTCTTAACTATGTTGCTCCTTTTACGCTGTGTGGATATGCTGC
TTTATAGCCTCTGTATCTAGCTATTGCTTCCCGTACGGCTTTCGTTTTCTCCTCCTTGTATAAATCCT
GGTTGCTGTCTCTTTTAGAGGAGTTGTGGCCCGTTGTCCGTCAACGTGGCGTGGTGTGCTCTGTGTTT
GCTGACGCAACCCCCACTGGCTGGGGCATTGCCACCACCTGTCAACTCCTTTCTGGGACTTTCGCTTT
CCCCCTCCCGATCGCCACGGCAGAACTCATCGCCGCCTGCCTTGCCCGCTGCTGGACAGGGGCTAGGT
TGCTGGGCACTGATAATTCCGTGGTGTTGTCTGTGCCTTCTAGTTGCCAGCCATCTGTTGTTTGCCCC
TCCCCCGTGCCTTCCTTGACCCTGGAAGGTGCCACTCCCACTGTCCTTTCCTAATAAAATGAGGAAAT
TGCATCGCATTGTCTGAGTAGGTGTCATTCTATTCTGGGGGGTGGGGTGGGGCAGGACAGCAAGGGGG
AGGATTGGGAAGACAATAGCAGGCATGCTGGGGATGCGGTGGGCTCTATGGCTCTAGAGCATGGCTAC
GTAGATAAGTAGCATGGCGGGTTAATCATTAACTACACCTGCAGGAGGAACCCCTAGTGATGGAGTTG
GCCACTCCCTCTCTGCGCGCTCGCTCGCTCACTGAGGCCGCCCGGGCAAAGCCCGGGCGTCGGGCGAC
CTTTGGTCGCCCGGCCTC
088 (Lanes 12-13;6203 bp; hFVIII ORF 985-5376)
GAGGCCGCCCGGGCAAAGCCCGGGCGTCGGGCGACCTTTGGTCGCCCGGCCTCAGTGAGCGAGCGAGC
GCGCAGAGAGGGAGTGGCCAACTCCATCACTAGGGGTTCCTCCTGCAGGTGTAGTTAATGATTAACCC
GCCATGCTACTTATCTACGTAGCCATGCTCTAGAGCCATAGAGCCCACCGCATCCCCAGCATGCCTGC
TATTGTCTTCCCAATCCTCCCCCTTGCTGTCCTGCCCCACCCCACCCCCCAGAATAGAATGACACCTA
CTCAGACAATGCGATGCAATTTCCTCATTTTATTAGGAAAGGACAGTGGGAGTGGCACCTTCCAGGGT
CAAGGAAGGCACGGGGGAGGGGCAAACAACAGATGGCTGGCAACTAGAAGGCACAGACAACACCACGG
AATTATCAGTGCCCAGCAACCTAGCCCCTGTCCAGCAGCGGGCAAGGCAGGCGGCGATGAGTTCTGCC
GTGGCGATCGGGAGGGGGAAAGCGAAAGTCCCAGAAAGGAGTTGACAGGTGGTGGCAATGCCCCAGCC
AGTGGGGGTTGCGTCAGCAAACACAGAGCACACCACGCCACGTTGACGGACAACGGGCCACAACTCCT
CTAAAAGAGACAGCAACCAGGATTTATACAAGGAGGAGAAAACGAAAGCCGTACGGGAAGCAATAGCT
AGATACAGAGGCTATAAAGCAGCATATCCACACAGCGTAAAAGGAGCAACATAGTTAAGAATATCAGT
CAATCTTTCACAAATTTTGTAATCCAGAGGTTGATTAACAGGAACAGAGCGTAAATAACGGGAAAGTT
TCTTAACATGTTTGTCTTGTGGCAATACACCTGAACTAGTAATTACATATCCCTAAAAATGTAAATGA
TTGCCCCACCATTTTGTTTTATTAACATTTAAATGTATACCCAAATCAAGAAAAACAGAACAAATATG
GGAATAAATGGCGGTAAGATGCTCTTAATTAATCAGTACAGGTCCTGGGCCTCACAGCCCAGCACCTC
CATCCTCAGGGCAATCTGGTGCACCCAGCTCTGGGGGTGAATCCTCAGGTATCTGGTCAGCAGGGGGG
GGTCCAGGCTGTTCACCACAGGGGTGAAGCTGTCCTGGTTGCCCTGGAACACCTTCACCTTGCCATTC
TGGAAGAACAGGGTCCACTGGTGGCCATCCTGGCTGCTGCTGATCAGGAACTCCTTCACATACATGCT
GGTCAGCAGGCTCTTCACCCCCTGGGTGGTCACCCCAGTCACCTTCATGGTCTTCTGGAAGTCCACCT
GCAGCCACTCCTTGGGGTTGTTGACCTGGGGCCTCCAGGCATTGCTCCTGCCCTGCAGGTGCAGCCTG
GCCTTGCTGGGGCTCCAGGTGGCAAACATGTTGGTGAAGTAGCTGCTGGCAGTGATCTGGGCATCAGA
GATGGCCTTGCTCTCCATGCCCAGGGGCATGCTGCAGCTGTTCAGGTCACAGCCCATCAGCTCCATCC
TCAGGGTGCTCCTGATGCTGTAGTGGGTGGGGTGCAGCCTGATGTATCTGGCAATGATGGGGGGGTTG
AAGATGTTGTGCTTGATGCCAGAGCTGTCCACATTGCCAAAGAACACCATCAGGGTGCCAGTGCTGTT
GCCCCTGTAGGTCTGCCACTTCTTGCCATCCAGGCTGTACATGATGATGAACTGGCTGATGTACAGGC
TGCTGAACTTCTGCCTGGCCCCCTGGGTCTTGATGCCATGGATGATCATGGGGGCCAGCAGGTCCACC
TTGATCCAGCTGAAGGGCTCCTTGGTGCTCCAGGCATTGATGCTGCCAGAGTAGTGCAGCCTGGCCAG
CTTGGGGGCCCACTGGCCATACTGGCCAGAGGCAGTGATCTGGAAGTCCCTGATGTGGCCAGAGGCCA
TGCCCAGGGGGGTCTGGCACTTGTTGCTGTACACCAGGAACAGGGTGCTCATGCCAGCATGCAGGTGC
TCCCCAATCAGGCACTCCACCCTCCAGATGCCAGCCTTGCTGGGCAGCATCTCCACAGTCTCAAACAC
CCCAGGGTACAGGTTGTACAGGGCCATCTTGTACTCCTCCTTCTTCCTCACAGTGAACACATGGCCAG
AGAAGTGGATGCTGTGGATGTTCTCATTGCTGCCCATGCTCAGCAGGTACCACCTGATCCTCTGGTCC
TGGGCCATCACCAGGCCAGGCAGGGTGTCCATGATGTAGCCATTGATGGCATGGAACCTGTAGTTCTC
CTTGAAGGTGGGGTCCTCCATCTGGATGTTGCAGGGGGCCCTGCAGTTCCTCTCCATGTTCTCAGTGA
AGTACCAGCTCTTGGTTTCATCAAAGATGGTGAAGAACAGGGCAAACTCCTGCACAGTCACCTGCCTG
CCATGGGCAGGGTTCAGGGTGTTGGTGTGGCACACCAGCAGGGGGCCAATCAGGCCAGAGTGCACATC
CTTCTCCAGGTCCACATCAGAGAAGTAGGCCCAGGCCTTGCAGTCAAACTCATCCTTGGTGGGGGCCA
TGTGGTGCTGCACCTTCCAGAAGTAGGTCTTGGTTTCATTGGGCTTCACAAAGTTCTTCCTGGGCTCA
GCCCCCTGCCTCTGGTCCTCCTCATAGCTGATCAGGCTGCTGTAGAAGCTGTAGGGCCTGCTGGCCTG
GTTCCTGAAGGTCACCATGATGTTGTCCTCCACCTCAGCCCTGATGTAGGGGCCCAGCAGGCCCAGGT
GCTCATTCAGCTCCCCTCTGTACAGGGGCTGGGTGAAGCTGCCATCAGTGAACTCCTGGAACACCACC
TTCTTGAACTGGGGCACAGAGCCAGACTGGGCCCTGTTCCTCAGCACATGGGGGCTGCTGCTCATGCC
ATAGTCCCACAGCCTCTCCACAGCAGCAATGAAGTAGTGCCTGGTCTTCTTCTGGAAGCTCCTGGGGC
TCTGGTTCTCGTCCTCGTCGTAGATGTCAAAGTCCTCCTTCTTCATCTCCACAGAGATGGTGTCATCA
TAGTCAATCTCCTCCTGGTCAGACTGCAGGGTGGTATTCTCTGGGATGGTGGTGGCATTGAACTGCTT
CTGCCTGGTGCTGGGGTGCCTGCTATTCTGGCTGAAGCTCCTGGGCTCAATGGCATTGTTCTTGCTCA
GCAGGTAGGCAGAGATGTCCTCATAGCTGTCCTCATAGTAGTCCCCAGTGTTCTTGTCACAGCTGGAG
ACTTTCAGCAGGGCAGTCATGCCCCTGTTCCTGAAGTCAGAGTTGTGGCAGCCCAGAATCCACAGGCC
AGGGTTCTCCATGCTCATGAACACAGTCTCCCCAGAGAAGGGGAACAGGGTCAGGGTGTCCTCATACA
CCATCTTGTGCTTGAAGGTGTAGCCAGAGAAGAACACAGACAGGAAGTCAGTCTGGGCCCCAATGCTC
AGGATGTACCAGTAGGCCACCTCATGCAGGCACACAGACAGCTGCAGGCTGTCAAACACATAGCCATT
GATGCTGTGCATGATGTTGCTGGCCTGGAACTCAGGGTCCTCCAGCTGCACCCCAGCAGGGTTGGGCA
GGAACCTCTGGATGTTCTCAGTCAGGTACCAGCTCCTGTTCTCATCAAACACAGAGAACAGGATCACA
TTCCTCTTGTCAGACATGATCTGGTTGCCCCTCTGGTCCACAGACTCCTTGTAGCAGATCAGCAGGGG
GCCAATCAGGCCAGAGGCCAGGTCCCTCTCCATGTTCACAAAGCTGCTGTAGTATCTGGTCAGGCACC
TGGGGTCAGACTTGGTGGGGCCATCCTCCACAGTCACAGTCCACTTGTACTTGAAGATCTCCCCAGGC
AGGATGGGGAAGTCCTTCAGGTGCTTCACCCCCTTGGGCAGCCTCCTGCTGTACAGGGGCCTCACATC
AGTGATGCCATGGGGGTAGATGTTGTAGGGCCTGCTGGCCTGGTTCTTGAAGATGATCAGCAGGGTGT
CCCCCACCTCCCCATACAGCAGGGGGCCCAGGATGCCAGACTCATGCTGGATGGCCTCCCTGGTCTTG
AAGGTTTCATCAGTGTAGGCCATGAACCTGACCTTCTTGTACTTCCTGCCAATCCTCTGGGGGCCATT
GTTCAGGTACTGGCTCTTGTAGCTCCTGTCATCAGGGGCCAGCACCAGGGGGGCATAGTCCCAGTCCT
CCTCCTCAGCAGCAATGTAGTGCACCCAGGTCTTGGGGTGCTTCTTGGCCACAGACCTGATCTGGATG
AAGCTGGGGCTGTTGTCATCATCAAACCTCACCACATCCATCTCAGAGTCAGTCAGGTCATCATCATA
GTCCTCAGCCTCCTCATTGTTCTTCATCCTCAGCTGGGGCTCCTCAGGGCAGCTGTCCACCTTCACAT
AGGCCTCCATGCCATCATGCTGGTGGCTGCTGATGTGGCAGAACAGCAGGAACTGGCCCAGGTCCATC
AGCAGGGTCTGGGCAGTCAGGAAGGTGATGGGGCTGATCTCCAGGCTGGCCTGCCTGTGGTTCCTGAC
CAGGAAGGTGTGGCCCTCCAGGAAGATGCTGTGCACCTCAGGGGTGGTGCCCATGCCAATCACATGCC
AGTACACAGACTTCCTGTGGCAGCCAATCAGGCCAGGCAGGCTCCTGTTCACATAGCCATTCACAGTG
TGCATCTTGGGCCAGGCCCTGGCAGAGGCAGCATCCCTGTCCTGCATCAGGCTGTTCTTGGTTTCAGA
GTGCCAGCTCTTGCCCTCATCAAACACAGCAAACAGCAGGATGAACTTGTGCAGGGTCTGGGTCTTCT
CCTTGGCCAGGCTGCCCTCCCTGCACACCAGCAGGGCCCCAATCAGGCCAGAGTTCAGGTCCTTCACC
AGGTCCACATGGCTCAGGTAGCTGTAGGTCAGGCACAGGGGGTCAGAGGCCATGGGGCCATTCTCCTT
CAGCACCTGCCACACATAGGTGTGGCTGCCCCCAGGGAACACCTTGTCATCCTCCTTCTCCCTCTGGC
TGGTCTGGTCATCATACTCAGCCCCCTCAGAGGCCTTCCAGTAGCTCACCCCCACAGCATGCAGGCTC
ACAGGGTGGCTGGCCATGTTCTTCAGGGTGATCACCACAGTGTCATACACCTCAGCCTGGATGGTGGG
GCCCAGCAGGCCCATCCAGGGGGGCCTGGGCTTGGCAATGTTGAACAGGTGGTCAGTGAACTCCACAA
ACAGGGTCTTCTTGTACACCACAGAGGTGTTGAAGGGGAAGCTCTTGGGCACTCTGGGGGGGAACCTG
1A1
GCATCCACAGGCAGCTCCCCCAGGTCAGACTGCATGTAGTCCCAGCTCAGCTCCACAGCCCCCAGGTA
GTATCTCCTGGTGGCAGAGAAGCAGAACCTCAGCAGGCACAGGAAGAAGCAGGTGCTCAGCTCAATCT
GCATGGTGGCGGCGTTTAAACCAACCTGAAAAAAAGTGATTTCAGGCAGGTGCTCCAGGTAATTAAAC
ATTAATACCCCACCAACCAACCATCCCTTAAACCCTTACCTCTTCAGGAGCTTGTGGATCTGTGTGAC
GGCTTCTCCTGGTGAAGGGGCTTTTATACCCCCTCCTTCCAACCCAGGCTGCTGATCCCTGCCAAGCT
GACTCCAAACCTGCTGATTCTGATTATTGACTTAGTCAACAAAAGGAGAATAAGTAACCTACACAAAT
ATGAACCTTGCCTAGGGAGATTAGAGTATCGGAACACTCGCTCTACGAAATGTGCAGACAGACGGGGA
TCCTCTAGAGCTACCTGCTGATCGCCCGGCCCCTGTTCAAACATGTCCTAATACTCTGTCTCTGCAAG
GGTCATCAGTAGTTTTCCATCTTACTCAACATCCTCCCAGTGGGTACCGTGGACTTAGCCCCTGTTTG
CTCCTCCGATAACTGGGGTGACCTTGGTTAATATTCACCAGCAGCCTCCCCCGGTGGACTTAGCCCCT
GTTTGCTCCTCCGATAACTGGGGTGACCTTGGTTAATATTCACCAGCAGCCTCCCCCGGTGGACTTAG
CCCCTGTTTGCTCCTCCGATAACTGGGGTGACCTTGGTTAATATTCACCAGCAGCCTCCCCCGCGGCC
GCGATAAGTAGCATGGCGGGTTAATCATTAACTACAAGGAACCCCTAGTGATGGAGCCTCAGCTTGGC
CACTCCCTCTCTGCGCGCTCGCTCGCTCACTGAGGCCGCCCGGGCAAAGCCCGGGCGTCGGGCGACCT
TTGGTCGCCCGGCIT
656 (Lanes 14-16; 6214 bp; hFVIII ORF 835-5226)
CTCACTGAGGCCGCCCGGGCAAAGCCCGGGCGTCGGGCGACCTTTGGTCGCCCGNCCTCAGTGAGCGA
GCGAGCGCGCAGAGAGGGAGTGGCCAAGCTGAGGCTCCATCACTAGGGGTTCCTTGTAGTTAATGATT
AACCCGCCATGCTACTTATCGCGGCCGCGGGGGAGGCTGCTGGTGAATATTAACCAAGGTCACCCCAG
TTATCGGAGGAGCAAACAGGGGCTAAGTCCACCGGGGGAGGCTGCTGGTGAATATTAACCAAGGTCAC
CCCAGTTATCGGAGGAGCAAACAGGGGCTAAGTCCACCGGGGGAGGCTGCTGGTGAATATTAACCAAG
GTCACCCCAGTTATCGGAGGAGCAAACAGGGGCTAAGTCCACGGTACCCACTGGGAGGATGTTGAGTA
AGATGGAAAACTACTGATGACCCTTGCAGAGACAGAGTATTAGGACATGTTTGAACAGGGGCCGGGCG
ATCAGCAGGTAGCTCTAGAGGATCCCCGTCTGTCTGCACATTTCGTAGAGCGAGTGTTCCGATACTCT
AATCTCCCTAGGCAAGGTTCATATTTGTGTAGGTTACTTATTCTCCTTTTGTTGACTAAGTCAATAAT
CAGAATCAGCAGGTTTGGAGTCAGCTTGGCAGGGATCAGCAGCCTGGGTTGGAAGGAGGGGGTATAAA
AGCCCCTTCACCAGGAGAAGCCGTCACACAGATCCACAAGCTCCTGAAGAGGTAAGGGTTTAAGGGAT
GGTTGGTTGGTGGGGTATTAATGTTTAATTACCTGGAGCACCTGCCTGAAATCACTTTTTTTCAGGTT
GGTTTAAACGCCGCCACCATGCAGATTGAGCTGAGCACCTGCTTCTTCCTGTGCCTGCTGAGGTTCTG
CTTCTCTGCCACCAGGAGATACTACCTGGGGGCTGTGGAGCTGAGCTGGGACTACATGCAGTCTGACC
TGGGGGAGCTGCCTGTGGATGCCAGGTTCCCCCCCAGAGTGCCCAAGAGCTTCCCCTTCAACACCTCT
GTGGTGTACAAGAAGACCCTGTTTGTGGAGTTCACTGACCACCTGTTCAACATTGCCAAGCCCAGGCC
CCCCTGGATGGGCCTGCTGGGCCCCACCATCCAGGCTGAGGTGTATGACACTGTGGTGATCACCCTGA
AGAACATGGCCAGCCACCCTGTGAGCCTGCATGCTGTGGGGGTGAGCTACTGGAAGGCCTCTGAGGGG
GCTGAGTATGATGACCAGACCAGCCAGAGGGAGAAGGAGGATGACAAGGTGTTCCCTGGGGGCAGCCA
CACCTATGTGTGGCAGGTGCTGAAGGAGAATGGCCCCATGGCCTCTGACCCCCTGTGCCTGACCTACA
GCTACCTGAGCCATGTGGACCTGGTGAAGGACCTGAACTCTGGCCTGATTGGGGCCCTGCTGGTGTGC
AGGGAGGGCAGCCTGGCCAAGGAGAAGACCCAGACCCTGCACAAGTTCATCCTGCTGTTTGCTGTGTT
TGATGAGGGCAAGAGCTGGCACTCTGAAACCAAGAACAGCCTGATGCAGGACAGGGATGCTGCCTCTG
CCAGGGCCTGGCCCAAGATGCACACTGTGAATGGCTATGTGAACAGGAGCCTGCCTGGCCTGATTGGC
TGCCACAGGAAGTCTGTGTACTGGCATGTGATTGGCATGGGCACCACCCCTGAGGTGCACAGCATCTT
CCTGGAGGGCCACACCTTCCTGGTCAGGAACCACAGGCAGGCCAGCCTGGAGATCAGCCCCATCACCT
TCCTGACTGCCCAGACCCTGCTGATGGACCTGGGCCAGTTCCTGCTGTTCTGCCACATCAGCAGCCAC
CAGCATGATGGCATGGAGGCCTATGTGAAGGTGGACAGCTGCCCTGAGGAGCCCCAGCTGAGGATGAA
GAACAATGAGGAGGCTGAGGACTATGATGATGACCTGACTGACTCTGAGATGGATGTGGTGAGGTTTG
ATGATGACAACAGCCCCAGCTTCATCCAGATCAGGTCTGTGGCCAAGAAGCACCCCAAGACCTGGGTG
CACTACATTGCTGCTGAGGAGGAGGACTGGGACTATGCCCCCCTGGTGCTGGCCCCTGATGACAGGAG
CTACAAGAGCCAGTACCTGAACAATGGCCCCCAGAGGATTGGCAGGAAGTACAAGAAGGTCAGGTTCA
TGGCCTACACTGATGAAACCTTCAAGACCAGGGAGGCCATCCAGCATGAGTCTGGCATCCTGGGCCCC
CTGCTGTATGGGGAGGTGGGGGACACCCTGCTGATCATCTTCAAGAACCAGGCCAGCAGGCCCTACAA
CATCTACCCCCATGGCATCACTGATGTGAGGCCCCTGTACAGCAGGAGGCTGCCCAAGGGGGTGAAGC
ACCTGAAGGACTTCCCCATCCTGCCTGGGGAGATCTTCAAGTACAAGTGGACTGTGACTGTGGAGGAT
GGCCCCACCAAGTCTGACCCCAGGTGCCTGACCAGATACTACAGCAGCTTTGTGAACATGGAGAGGGA
CCTGGCCTCTGGCCTGATTGGCCCCCTGCTGATCTGCTACAAGGAGTCTGTGGACCAGAGGGGCAACC
AGATCATGTCTGACAAGAGGAATGTGATCCTGTTCTCTGTGTTTGATGAGAACAGGAGCTGGTACCTG
ACTGAGAACATCCAGAGGTTCCTGCCCAACCCTGCTGGGGTGCAGCTGGAGGACCCTGAGTTCCAGGC
CAGCAACATCATGCACAGCATCAATGGCTATGTGTTTGACAGCCTGCAGCTGTCTGTGTGCCTGCATG
AGGTGGCCTACTGGTACATCCTGAGCATTGGGGCCCAGACTGACTTCCTGTCTGTGTTCTTCTCTGGC
TACACCTTCAAGCACAAGATGGTGTATGAGGACACCCTGACCCTGTTCCCCTTCTCTGGGGAGACTGT
GTTCATGAGCATGGAGAACCCTGGCCTGTGGATTCTGGGCTGCCACAACTCTGACTTCAGGAACAGGG
GCATGACTGCCCTGCTGAAAGTCTCCAGCTGTGACAAGAACACTGGGGACTACTATGAGGACAGCTAT
GAGGACATCTCTGCCTACCTGCTGAGCAAGAACAATGCCATTGAGCCCAGGAGCTTCAGCCAGAATAG
CAGGCACCCCAGCACCAGGCAGAAGCAGTTCAATGCCACCACCATCCCAGAGAATACCACCCTGCAGT
CTGACCAGGAGGAGATTGACTATGATGACACCATCTCTGTGGAGATGAAGAAGGAGGACTTTGACATC
TACGACGAGGACGAGAACCAGAGCCCCAGGAGCTTCCAGAAGAAGACCAGGCACTACTTCATTGCTGC
TGTGGAGAGGCTGTGGGACTATGGCATGAGCAGCAGCCCCCATGTGCTGAGGAACAGGGCCCAGTCTG
GCTCTGTGCCCCAGTTCAAGAAGGTGGTGTTCCAGGAGTTCACTGATGGCAGCTTCACCCAGCCCCTG
TACAGAGGGGAGCTGAATGAGCACCTGGGCCTGCTGGGCCCCTACATCAGGGCTGAGGTGGAGGACAA
CATCATGGTGACCTTCAGGAACCAGGCCAGCAGGCCCTACAGCTTCTACAGCAGCCTGATCAGCTATG
AGGAGGACCAGAGGCAGGGGGCTGAGCCCAGGAAGAACTTTGTGAAGCCCAATGAAACCAAGACCTAC
TTCTGGAAGGTGCAGCACCACATGGCCCCCACCAAGGATGAGTTTGACTGCAAGGCCTGGGCCTACTT
CTCTGATGTGGACCTGGAGAAGGATGTGCACTCTGGCCTGATTGGCCCCCTGCTGGTGTGCCACACCA
ACACCCTGAACCCTGCCCATGGCAGGCAGGTGACTGTGCAGGAGTTTGCCCTGTTCTTCACCATCTTT
GATGAAACCAAGAGCTGGTACTTCACTGAGAACATGGAGAGGAACTGCAGGGCCCCCTGCAACATCCA
GATGGAGGACCCCACCTTCAAGGAGAACTACAGGTTCCATGCCATCAATGGCTACATCATGGACACCC
TGCCTGGCCTGGTGATGGCCCAGGACCAGAGGATCAGGTGGTACCTGCTGAGCATGGGCAGCAATGAG
AACATCCACAGCATCCACTTCTCTGGCCATGTGTTCACTGTGAGGAAGAAGGAGGAGTACAAGATGGC
CCTGTACAACCTGTACCCTGGGGTGTTTGAGACTGTGGAGATGCTGCCCAGCAAGGCTGGCATCTGGA
GGGTGGAGTGCCTGATTGGGGAGCACCTGCATGCTGGCATGAGCACCCTGTTCCTGGTGTACAGCAAC
AAGTGCCAGACCCCCCTGGGCATGGCCTCTGGCCACATCAGGGACTTCCAGATCACTGCCTCTGGCCA
GTATGGCCAGTGGGCCCCCAAGCTGGCCAGGCTGCACTACTCTGGCAGCATCAATGCCTGGAGCACCA
AGGAGCCCTTCAGCTGGATCAAGGTGGACCTGCTGGCCCCCATGATCATCCATGGCATCAAGACCCAG
GGGGCCAGGCAGAAGTTCAGCAGCCTGTACATCAGCCAGTTCATCATCATGTACAGCCTGGATGGCAA
GAAGTGGCAGACCTACAGGGGCAACAGCACTGGCACCCTGATGGTGTTCTTTGGCAATGTGGACAGCT
CTGGCATCAAGCACAACATCTTCAACCCCCCCATCATTGCCAGATACATCAGGCTGCACCCCACCCAC
TACAGCATCAGGAGCACCCTGAGGATGGAGCTGATGGGCTGTGACCTGAACAGCTGCAGCATGCCCCT
GGGCATGGAGAGCAAGGCCATCTCTGATGCCCAGATCACTGCCAGCAGCTACTTCACCAACATGTTTG
CCACCTGGAGCCCCAGCAAGGCCAGGCTGCACCTGCAGGGCAGGAGCAATGCCTGGAGGCCCCAGGTC
AACAACCCCAAGGAGTGGCTGCAGGTGGACTTCCAGAAGACCATGAAGGTGACTGGGGTGACCACCCA
GGGGGTGAAGAGCCTGCTGACCAGCATGTATGTGAAGGAGTTCCTGATCAGCAGCAGCCAGGATGGCC
ACCAGTGGACCCTGTTCTTCCAGAATGGCAAGGTGAAGGTGTTCCAGGGCAACCAGGACAGCTTCACC
CCTGTGGTGAACAGCCTGGACCCCCCCCTGCTGACCAGATACCTGAGGATTCACCCCCAGAGCTGGGT
GCACCAGATTGCCCTGAGGATGGAGGTGCTGGGCTGTGAGGCCCAGGACCTGTACTGATTAATTAAGA
GCATCTTACCGCCATTTATTCCCATATTTGTTCTGTTTTTCTTGATTTGGGTATACATTTAAATGTTA
ATAAAACAAAATGGTGGGGCAATCATTTACATTTTTAGGGATATGTAATTACTAGTTCAGGTGTATTG
CCACAAGACAAACATGTTAAGAAACTTTCCCGTTATTTACGCTCTGTTCCTGTTAATCAACCTCTGGA
TTACAAAATTTGTGAAAGATTGACTGATATTCTTAACTATGTTGCTCCTTTTACGCTGTGTGGATATG
CTGCTTTATAGCCTCTGTATCTAGCTATTGCTTCCCGTACGGCTTTCGTTTTCTCCTCCTTGTATAAA
TCCTGGTTGCTGTCTCTTTTAGAGGAGTTGTGGCCCGTTGTCCGTCAACGTGGCGTGGTGTGCTCTGT
GTTTGCTGACGCAACCCCCACTGGCTGGGGCATTGCCACCACCTGTCAACTCCTTTCTGGGACTTTCG
CTTTCCCCCTCCCGATCGCCACGGCAGAACTCATCGCCGCCTGCCTTGCCCGCTGCTGGACAGGGGCT
AGGTTGCTGGGCACTGATAATTCCGTGGTGTTGTCTGTGCCTTCTAGTTGCCAGCCATCTGTTGTTTG
CCCCTCCCCCGTGCCTTCCTTGACCCTGGAAGGTGCCACTCCCACTGTCCTTTCCTAATAAAATGAGG
AAATTGCATCGCATTGTCTGAGTAGGTGTCATTCTATTCTGGGGGGTGGGGTGGGGCAGGACAGCAAG
GGGGAGGATTGGGAAGACAATAGCAGGCATGCTGGGGATGCGGTGGGCTCTATGGCTCTAGAGCATGG
CTACGTAGATAAGTAGCATGGCGGGTTAATCATTAACTACACCTGCAGGAGGAACCCCTAGTGATGGA
GTTGGCCACTCCCTCTCTGCGCGCTCGCTCGCTCACTGAGGCCGGGCGACCAAAGGTCGCCCGACGCC
CGGGCTTTGCCCGGGCGGCCTCAGTG
042 (Lanes 15-16; 6201 bp; hFVIII ORF 985-5376)
GAGGCCGCCCGGGCAAAGCCCGGGCGTCGGGCGACCTTTGGTCGCCCGGCCTCAGTGAGCGAGCGAGC
GCGCAGAGAGGGAGTGGCCAACTCCATCACTAGGGGTTCCTCCTGCAGGTGTAGTTAATGATTAACCC
GCCATGCTACTTATCTACGTAGCCATGCTCTAGAGCCATAGAGCCCACCGCATCCCCAGCATGCCTGC
TATTGTCTTCCCAATCCTCCCCCTTGCTGTCCTGCCCCACCCCACCCCCCAGAATAGAATGACACCTA
CTCAGACAATGCGATGCAATTTCCTCATTTTATTAGGAAAGGACAGTGGGAGTGGCACCTTCCAGGGT
CAAGGAAGGCACGGGGGAGGGGCAAACAACAGATGGCTGGCAACTAGAAGGCACAGACAACACCACGG
AATTATCAGTGCCCAGCAACCTAGCCCCTGTCCAGCAGCGGGCAAGGCAGGCGGCGATGAGTTCTGCC
GTGGCGATCGGGAGGGGGAAAGCGAAAGTCCCAGAAAGGAGTTGACAGGTGGTGGCAATGCCCCAGCC
AGTGGGGGTTGCGTCAGCAAACACAGAGCACACCACGCCACGTTGACGGACAACGGGCCACAACTCCT
CTAAAAGAGACAGCAACCAGGATTTATACAAGGAGGAGAAAACGAAAGCCGTACGGGAAGCAATAGCT
AGATACAGAGGCTATAAAGCAGCATATCCACACAGCGTAAAAGGAGCAACATAGTTAAGAATATCAGT
CAATCTTTCACAAATTTTGTAATCCAGAGGTTGATTAACAGGAACAGAGCGTAAATAACGGGAAAGTT
TCTTAACATGTTTGTCTTGTGGCAATACACCTGAACTAGTAATTACATATCCCTAAAAATGTAAATGA
TTGCCCCACCATTTTGTTTTATTAACATTTAAATGTATACCCAAATCAAGAAAAACAGAACAAATATG
GGAATAAATGGCGGTAAGATGCTCTTAATTAATCAGTACAGGTCCTGGGCCTCACAGCCCAGCACCTC
CATCCTCAGGGCAATCTGGTGCACCCAGCTCTGGGGGTGAATCCTCAGGTATCTGGTCAGCAGGGGGG
GGTCCAGGCTGTTCACCACAGGGGTGAAGCTGTCCTGGTTGCCCTGGAACACCTTCACCTTGCCATTC
TGGAAGAACAGGGTCCACTGGTGGCCATCCTGGCTGCTGCTGATCAGGAACTCCTTCACATACATGCT
GGTCAGCAGGCTCTTCACCCCCTGGGTGGTCACCCCAGTCACCTTCATGGTCTTCTGGAAGTCCACCT
GCAGCCACTCCTTGGGGTTGTTGACCTGGGGCCTCCAGGCATTGCTCCTGCCCTGCAGGTGCAGCCTG
GCCTTGCTGGGGCTCCAGGTGGCAAACATGTTGGTGAAGTAGCTGCTGGCAGTGATCTGGGCATCAGA
GATGGCCTTGCTCTCCATGCCCAGGGGCATGCTGCAGCTGTTCAGGTCACAGCCCATCAGCTCCATCC
TCAGGGTGCTCCTGATGCTGTAGTGGGTGGGGTGCAGCCTGATGTATCTGGCAATGATGGGGGGGTTG
AAGATGTTGTGCTTGATGCCAGAGCTGTCCACATTGCCAAAGAACACCATCAGGGTGCCAGTGCTGTT
GCCCCTGTAGGTCTGCCACTTCTTGCCATCCAGGCTGTACATGATGATGAACTGGCTGATGTACAGGC
TGCTGAACTTCTGCCTGGCCCCCTGGGTCTTGATGCCATGGATGATCATGGGGGCCAGCAGGTCCACC
TTGATCCAGCTGAAGGGCTCCTTGGTGCTCCAGGCATTGATGCTGCCAGAGTAGTGCAGCCTGGCCAG
CTTGGGGGCCCACTGGCCATACTGGCCAGAGGCAGTGATCTGGAAGTCCCTGATGTGGCCAGAGGCCA
TGCCCAGGGGGGTCTGGCACTTGTTGCTGTACACCAGGAACAGGGTGCTCATGCCAGCATGCAGGTGC
TCCCCAATCAGGCACTCCACCCTCCAGATGCCAGCCTTGCTGGGCAGCATCTCCACAGTCTCAAACAC
CCCAGGGTACAGGTTGTACAGGGCCATCTTGTACTCCTCCTTCTTCCTCACAGTGAACACATGGCCAG
AGAAGTGGATGCTGTGGATGTTCTCATTGCTGCCCATGCTCAGCAGGTACCACCTGATCCTCTGGTCC
TGGGCCATCACCAGGCCAGGCAGGGTGTCCATGATGTAGCCATTGATGGCATGGAACCTGTAGTTCTC
CTTGAAGGTGGGGTCCTCCATCTGGATGTTGCAGGGGGCCCTGCAGTTCCTCTCCATGTTCTCAGTGA
AGTACCAGCTCTTGGTTTCATCAAAGATGGTGAAGAACAGGGCAAACTCCTGCACAGTCACCTGCCTG
CCATGGGCAGGGTTCAGGGTGTTGGTGTGGCACACCAGCAGGGGGCCAATCAGGCCAGAGTGCACATC
CTTCTCCAGGTCCACATCAGAGAAGTAGGCCCAGGCCTTGCAGTCAAACTCATCCTTGGTGGGGGCCA
TGTGGTGCTGCACCTTCCAGAAGTAGGTCTTGGTTTCATTGGGCTTCACAAAGTTCTTCCTGGGCTCA
GCCCCCTGCCTCTGGTCCTCCTCATAGCTGATCAGGCTGCTGTAGAAGCTGTAGGGCCTGCTGGCCTG
GTTCCTGAAGGTCACCATGATGTTGTCCTCCACCTCAGCCCTGATGTAGGGGCCCAGCAGGCCCAGGT
GCTCATTCAGCTCCCCTCTGTACAGGGGCTGGGTGAAGCTGCCATCAGTGAACTCCTGGAACACCACC
TTCTTGAACTGGGGCACAGAGCCAGACTGGGCCCTGTTCCTCAGCACATGGGGGCTGCTGCTCATGCC
ATAGTCCCACAGCCTCTCCACAGCAGCAATGAAGTAGTGCCTGGTCTTCTTCTGGAAGCTCCTGGGGC
TCTGGTTCTCGTCCTCGTCGTAGATGTCAAAGTCCTCCTTCTTCATCTCCACAGAGATGGTGTCATCA
TAGTCAATCTCCTCCTGGTCAGACTGCAGGGTGGTATTCTCTGGGATGGTGGTGGCATTGAACTGCTT
CTGCCTGGTGCTGGGGTGCCTGCTATTCTGGCTGAAGCTCCTGGGCTCAATGGCATTGTTCTTGCTCA
GCAGGTAGGCAGAGATGTCCTCATAGCTGTCCTCATAGTAGTCCCCAGTGTTCTTGTCACAGCTGGAG
ACTTTCAGCAGGGCAGTCATGCCCCTGTTCCTGAAGTCAGAGTTGTGGCAGCCCAGAATCCACAGGCC
AGGGTTCTCCATGCTCATGAACACAGTCTCCCCAGAGAAGGGGAACAGGGTCAGGGTGTCCTCATACA
CCATCTTGTGCTTGAAGGTGTAGCCAGAGAAGAACACAGACAGGAAGTCAGTCTGGGCCCCAATGCTC
AGGATGTACCAGTAGGCCACCTCATGCAGGCACACAGACAGCTGCAGGCTGTCAAACACATAGCCATT
GATGCTGTGCATGATGTTGCTGGCCTGGAACTCAGGGTCCTCCAGCTGCACCCCAGCAGGGTTGGGCA
GGAACCTCTGGATGTTCTCAGTCAGGTACCAGCTCCTGTTCTCATCAAACACAGAGAACAGGATCACA
TTCCTCTTGTCAGACATGATCTGGTTGCCCCTCTGGTCCACAGACTCCTTGTAGCAGATCAGCAGGGG
GCCAATCAGGCCAGAGGCCAGGTCCCTCTCCATGTTCACAAAGCTGCTGTAGTATCTGGTCAGGCACC
TGGGGTCAGACTTGGTGGGGCCATCCTCCACAGTCACAGTCCACTTGTACTTGAAGATCTCCCCAGGC
AGGATGGGGAAGTCCTTCAGGTGCTTCACCCCCTTGGGCAGCCTCCTGCTGTACAGGGGCCTCACATC
AGTGATGCCATGGGGGTAGATGTTGTAGGGCCTGCTGGCCTGGTTCTTGAAGATGATCAGCAGGGTGT
CCCCCACCTCCCCATACAGCAGGGGGCCCAGGATGCCAGACTCATGCTGGATGGCCTCCCTGGTCTTG
AAGGTTTCATCAGTGTAGGCCATGAACCTGACCTTCTTGTACTTCCTGCCAATCCTCTGGGGGCCATT
GTTCAGGTACTGGCTCTTGTAGCTCCTGTCATCAGGGGCCAGCACCAGGGGGGCATAGTCCCAGTCCT
CCTCCTCAGCAGCAATGTAGTGCACCCAGGTCTTGGGGTGCTTCTTGGCCACAGACCTGATCTGGATG
AAGCTGGGGCTGTTGTCATCATCAAACCTCACCACATCCATCTCAGAGTCAGTCAGGTCATCATCATA
GTCCTCAGCCTCCTCATTGTTCTTCATCCTCAGCTGGGGCTCCTCAGGGCAGCTGTCCACCTTCACAT
AGGCCTCCATGCCATCATGCTGGTGGCTGCTGATGTGGCAGAACAGCAGGAACTGGCCCAGGTCCATC
AGCAGGGTCTGGGCAGTCAGGAAGGTGATGGGGCTGATCTCCAGGCTGGCCTGCCTGTGGTTCCTGAC
CAGGAAGGTGTGGCCCTCCAGGAAGATGCTGTGCACCTCAGGGGTGGTGCCCATGCCAATCACATGCC
AGTACACAGACTTCCTGTGGCAGCCAATCAGGCCAGGCAGGCTCCTGTTCACATAGCCATTCACAGTG
TGCATCTTGGGCCAGGCCCTGGCAGAGGCAGCATCCCTGTCCTGCATCAGGCTGTTCTTGGTTTCAGA
GTGCCAGCTCTTGCCCTCATCAAACACAGCAAACAGCAGGATGAACTTGTGCAGGGTCTGGGTCTTCT
CCTTGGCCAGGCTGCCCTCCCTGCACACCAGCAGGGCCCCAATCAGGCCAGAGTTCAGGTCCTTCACC
AGGTCCACATGGCTCAGGTAGCTGTAGGTCAGGCACAGGGGGTCAGAGGCCATGGGGCCATTCTCCTT
CAGCACCTGCCACACATAGGTGTGGCTGCCCCCAGGGAACACCTTGTCATCCTCCTTCTCCCTCTGGC
TGGTCTGGTCATCATACTCAGCCCCCTCAGAGGCCTTCCAGTAGCTCACCCCCACAGCATGCAGGCTC
ACAGGGTGGCTGGCCATGTTCTTCAGGGTGATCACCACAGTGTCATACACCTCAGCCTGGATGGTGGG
GCCCAGCAGGCCCATCCAGGGGGGCCTGGGCTTGGCAATGTTGAACAGGTGGTCAGTGAACTCCACAA
ACAGGGTCTTCTTGTACACCACAGAGGTGTTGAAGGGGAAGCTCTTGGGCACTCTGGGGGGGAACCTG
GCATCCACAGGCAGCTCCCCCAGGTCAGACTGCATGTAGTCCCAGCTCAGCTCCACAGCCCCCAGGTA
GTATCTCCTGGTGGCAGAGAAGCAGAACCTCAGCAGGCACAGGAAGAAGCAGGTGCTCAGCTCAATCT
GCATGGTGGCGGCGTTTAAACCAACCTGAAAAAAAGTGATTTCAGGCAGGTGCTCCAGGTAATTAAAC
ATTAATACCCCACCAACCAACCATCCCTTAAACCCTTACCTCTTCAGGAGCTTGTGGATCTGTGTGAC
GGCTTCTCCTGGTGAAGGGGCTTTTATACCCCCTCCTTCCAACCCAGGCTGCTGATCCCTGCCAAGCT
GACTCCAAACCTGCTGATTCTGATTATTGACTTAGTCAACAAAAGGAGAATAAGTAACCTACACAAAT
ATGAACCTTGCCTAGGGAGATTAGAGTATCGGAACACTCGCTCTACGAAATGTGCAGACAGACGGGGA
TCCTCTAGAGCTACCTGCTGATCGCCCGGCCCCTGTTCAAACATGTCCTAATACTCTGTCTCTGCAAG
GGTCATCAGTAGTTTTCCATCTTACTCAACATCCTCCCAGTGGGTACCGTGGACTTAGCCCCTGTTTG
CTCCTCCGATAACTGGGGTGACCTTGGTTAATATTCACCAGCAGCCTCCCCCGGTGGACTTAGCCCCT
GTTTGCTCCTCCGATAACTGGGGTGACCTTGGTTAATATTCACCAGCAGCCTCCCCCGGTGGACTTAG
CCCCTGTTTGCTCCTCCGATAACTGGGGTGACCTTGGTTAATATTCACCAGCAGCCTCCCCCGCGGCC
GCGATAAGTAGCATGGCGGGTTAATCATTAACTACAAGGAACCCCTAGTGATGGAGCCTCAGCTTGGC
CACTCCCTCTCTGCGCGCTCGCTCGCTCACTGAGGCCGCCCGGGCAAAGCCCGGGCGTCGGGCGACCT
TTGGTCGCCCGIC

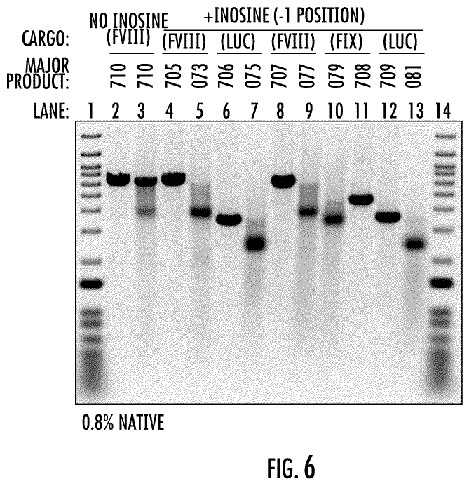

FIG. 6 shows results demonstrating that the universal Endonuclease V-mediated synthesis protocol enables efficient ssDNA conversion across constructs. Multiple ceDNA with unique internal sequences were generated via RAMP. All ceDNA contained a left ITR with inosine present at the −1 position and a right ITR with an extended A-stem (SO-238; SEQ ID NO: 14) (lanes 3, 5, 7, 10, 11) ceDNA lacking inosine served as a control for Endonuclease V activity (lane 2). All ceDNA were subjected to Endonuclease V-mediated ssDNA synthesis (lanes 2, 4, 6, 8, 9, 12).

TABLE 9

| Lane | | | | ssDNA (if |
|---|---|---|---|---|
| number | Description | Reaction | ceDNA | applicable) |
| 1 | Size marker | NA | NA | NA |
| 2 | No inosine contained | RAMP | 710 | NA |
| | within ceDNA | | | |
| 3 | No inosine contained | ssDNA | 710 | NA |
| | within ceDNA | synthesis | | |
| 4 | Inosine at −1 position of | RAMP | 705 | NA |
| | left ITR within ceDNA | | | |
| 5 | Inosine at −1 position of | ssDNA | 705 | 073 |
| | left ITR within ceDNA | synthesis | | |
| 6 | Inosine at −1 position of | RAMP | 706 | NA |
| | left ITR within ceDNA | | | |
| 7 | Inosine at −1 position of | ssDNA | 706 | 075 |
| | left ITR within ceDNA | synthesis | | |
| 8 | Inosine at −1 position of | RAMP | 707 | NA |
| | left ITR within ceDNA | | | |
| 9 | Inosine at −1 position of | ssDNA | 707 | 077 |
| | left ITR within ceDNA | synthesis | | |
| 10 | Inosine at −1 position of | ssDNA | 708 | 079 |
| | left ITR within ceDNA | synthesis | | |
| 11 | Inosine at −1 position of | RAMP | 708 | NA |
| | left ITR within ceDNA | | | |
| 12 | Inosine at −1 position of | RAMP | 709 | NA |
| | left ITR within ceDNA | | | |
| 13 | Inosine at −1 position of | ssDNA | 709 | 081 |
| | left ITR within ceDNA | synthesis | | |
| 14 | Size marker | NA | NA | NA |

SO-238;
SEQ ID NO: 14
ATACCAGCTTATTCAATTGGTCTCGCACTTACCTCTACACACTCGGCGCCAATAAAAAGACAGAATAA
AAGACTGAGATGGACGGGCAACTGCGTCTCATTCACGTTAGAGACTCCAACCGTCCATCTCAGAATTT
TATTCTGTCTTTTTATTGGCGCCGAGTGTGTAGAGGTAAGTGCGAGACCAGATAGTAAGTGCAATC
710 (Lanes 2-4; 6206 bp; hFVIII ORF 831-5222)
See above
705 (Lanes 4-5; 6214 bp; hFVIII ORF 831-5222)
CTGAGGCCGCCCGGGCAAAGCCCGGGCGTCGGGCGACCTTTGGTCGCCCGGCCTCANTGAGCGAGCGA
GCGCGCAGAGAGGGAGTGGCCAAGCTGAGGCTCCATCACTAGGGGTTCCTTGTAGTTAATGATTAACC
CGCCATGCTACTTATCGCGGCCGCGGGGGAGGCTGCTGGTGAATATTAACCAAGGTCACCCCAGTTAT
CGGAGGAGCAAACAGGGGCTAAGTCCACCGGGGGAGGCTGCTGGTGAATATTAACCAAGGTCACCCCA
GTTATCGGAGGAGCAAACAGGGGCTAAGTCCACCGGGGGAGGCTGCTGGTGAATATTAACCAAGGTCA
CCCCAGTTATCGGAGGAGCAAACAGGGGCTAAGTCCACGGTACCCACTGGGAGGATGTTGAGTAAGAT
GGAAAACTACTGATGACCCTTGCAGAGACAGAGTATTAGGACATGTTTGAACAGGGGCCGGGCGATCA
GCAGGTAGCTCTAGAGGATCCCCGTCTGTCTGCACATTTCGTAGAGCGAGTGTTCCGATACTCTAATC
TCCCTAGGCAAGGTTCATATTTGTGTAGGTTACTTATTCTCCTTTTGTTGACTAAGTCAATAATCAGA
ATCAGCAGGTTTGGAGTCAGCTTGGCAGGGATCAGCAGCCTGGGTTGGAAGGAGGGGGTATAAAAGCC
CCTTCACCAGGAGAAGCCGTCACACAGATCCACAAGCTCCTGAAGAGGTAAGGGTTTAAGGGATGGTT
GGTTGGTGGGGTATTAATGTTTAATTACCTGGAGCACCTGCCTGAAATCACTTTTTTTCAGGTTGGTT
TAAACGCCGCCACCATGCAGATTGAGCTGAGCACCTGCTTCTTCCTGTGCCTGCTGAGGTTCTGCTTC
TCTGCCACCAGGAGATACTACCTGGGGGCTGTGGAGCTGAGCTGGGACTACATGCAGTCTGACCTGGG
GGAGCTGCCTGTGGATGCCAGGTTCCCCCCCAGAGTGCCCAAGAGCTTCCCCTTCAACACCTCTGTGG
TGTACAAGAAGACCCTGTTTGTGGAGTTCACTGACCACCTGTTCAACATTGCCAAGCCCAGGCCCCCC
TGGATGGGCCTGCTGGGCCCCACCATCCAGGCTGAGGTGTATGACACTGTGGTGATCACCCTGAAGAA
CATGGCCAGCCACCCTGTGAGCCTGCATGCTGTGGGGGTGAGCTACTGGAAGGCCTCTGAGGGGGCTG
AGTATGATGACCAGACCAGCCAGAGGGAGAAGGAGGATGACAAGGTGTTCCCTGGGGGCAGCCACACC
TATGTGTGGCAGGTGCTGAAGGAGAATGGCCCCATGGCCTCTGACCCCCTGTGCCTGACCTACAGCTA
CCTGAGCCATGTGGACCTGGTGAAGGACCTGAACTCTGGCCTGATTGGGGCCCTGCTGGTGTGCAGGG
AGGGCAGCCTGGCCAAGGAGAAGACCCAGACCCTGCACAAGTTCATCCTGCTGTTTGCTGTGTTTGAT
GAGGGCAAGAGCTGGCACTCTGAAACCAAGAACAGCCTGATGCAGGACAGGGATGCTGCCTCTGCCAG
GGCCTGGCCCAAGATGCACACTGTGAATGGCTATGTGAACAGGAGCCTGCCTGGCCTGATTGGCTGCC
ACAGGAAGTCTGTGTACTGGCATGTGATTGGCATGGGCACCACCCCTGAGGTGCACAGCATCTTCCTG
GAGGGCCACACCTTCCTGGTCAGGAACCACAGGCAGGCCAGCCTGGAGATCAGCCCCATCACCTTCCT
GACTGCCCAGACCCTGCTGATGGACCTGGGCCAGTTCCTGCTGTTCTGCCACATCAGCAGCCACCAGC
ATGATGGCATGGAGGCCTATGTGAAGGTGGACAGCTGCCCTGAGGAGCCCCAGCTGAGGATGAAGAAC
AATGAGGAGGCTGAGGACTATGATGATGACCTGACTGACTCTGAGATGGATGTGGTGAGGTTTGATGA
TGACAACAGCCCCAGCTTCATCCAGATCAGGTCTGTGGCCAAGAAGCACCCCAAGACCTGGGTGCACT
ACATTGCTGCTGAGGAGGAGGACTGGGACTATGCCCCCCTGGTGCTGGCCCCTGATGACAGGAGCTAC
AAGAGCCAGTACCTGAACAATGGCCCCCAGAGGATTGGCAGGAAGTACAAGAAGGTCAGGTTCATGGC
CTACACTGATGAAACCTTCAAGACCAGGGAGGCCATCCAGCATGAGTCTGGCATCCTGGGCCCCCTGC
TGTATGGGGAGGTGGGGGACACCCTGCTGATCATCTTCAAGAACCAGGCCAGCAGGCCCTACAACATC
TACCCCCATGGCATCACTGATGTGAGGCCCCTGTACAGCAGGAGGCTGCCCAAGGGGGTGAAGCACCT
GAAGGACTTCCCCATCCTGCCTGGGGAGATCTTCAAGTACAAGTGGACTGTGACTGTGGAGGATGGCC
CCACCAAGTCTGACCCCAGGTGCCTGACCAGATACTACAGCAGCTTTGTGAACATGGAGAGGGACCTG
GCCTCTGGCCTGATTGGCCCCCTGCTGATCTGCTACAAGGAGTCTGTGGACCAGAGGGGCAACCAGAT
CATGTCTGACAAGAGGAATGTGATCCTGTTCTCTGTGTTTGATGAGAACAGGAGCTGGTACCTGACTG
AGAACATCCAGAGGTTCCTGCCCAACCCTGCTGGGGTGCAGCTGGAGGACCCTGAGTTCCAGGCCAGC
AACATCATGCACAGCATCAATGGCTATGTGTTTGACAGCCTGCAGCTGTCTGTGTGCCTGCATGAGGT
GGCCTACTGGTACATCCTGAGCATTGGGGCCCAGACTGACTTCCTGTCTGTGTTCTTCTCTGGCTACA
CCTTCAAGCACAAGATGGTGTATGAGGACACCCTGACCCTGTTCCCCTTCTCTGGGGAGACTGTGTTC
ATGAGCATGGAGAACCCTGGCCTGTGGATTCTGGGCTGCCACAACTCTGACTTCAGGAACAGGGGCAT
GACTGCCCTGCTGAAAGTCTCCAGCTGTGACAAGAACACTGGGGACTACTATGAGGACAGCTATGAGG
ACATCTCTGCCTACCTGCTGAGCAAGAACAATGCCATTGAGCCCAGGAGCTTCAGCCAGAATAGCAGG
CACCCCAGCACCAGGCAGAAGCAGTTCAATGCCACCACCATCCCAGAGAATACCACCCTGCAGTCTGA
CCAGGAGGAGATTGACTATGATGACACCATCTCTGTGGAGATGAAGAAGGAGGACTTTGACATCTACG
ACGAGGACGAGAACCAGAGCCCCAGGAGCTTCCAGAAGAAGACCAGGCACTACTTCATTGCTGCTGTG
GAGAGGCTGTGGGACTATGGCATGAGCAGCAGCCCCCATGTGCTGAGGAACAGGGCCCAGTCTGGCTC
TGTGCCCCAGTTCAAGAAGGTGGTGTTCCAGGAGTTCACTGATGGCAGCTTCACCCAGCCCCTGTACA
GAGGGGAGCTGAATGAGCACCTGGGCCTGCTGGGCCCCTACATCAGGGCTGAGGTGGAGGACAACATC
ATGGTGACCTTCAGGAACCAGGCCAGCAGGCCCTACAGCTTCTACAGCAGCCTGATCAGCTATGAGGA
GGACCAGAGGCAGGGGGCTGAGCCCAGGAAGAACTTTGTGAAGCCCAATGAAACCAAGACCTACTTCT
GGAAGGTGCAGCACCACATGGCCCCCACCAAGGATGAGTTTGACTGCAAGGCCTGGGCCTACTTCTCT
GATGTGGACCTGGAGAAGGATGTGCACTCTGGCCTGATTGGCCCCCTGCTGGTGTGCCACACCAACAC
CCTGAACCCTGCCCATGGCAGGCAGGTGACTGTGCAGGAGTTTGCCCTGTTCTTCACCATCTTTGATG
AAACCAAGAGCTGGTACTTCACTGAGAACATGGAGAGGAACTGCAGGGCCCCCTGCAACATCCAGATG
GAGGACCCCACCTTCAAGGAGAACTACAGGTTCCATGCCATCAATGGCTACATCATGGACACCCTGCC
TGGCCTGGTGATGGCCCAGGACCAGAGGATCAGGTGGTACCTGCTGAGCATGGGCAGCAATGAGAACA
TCCACAGCATCCACTTCTCTGGCCATGTGTTCACTGTGAGGAAGAAGGAGGAGTACAAGATGGCCCTG
TACAACCTGTACCCTGGGGTGTTTGAGACTGTGGAGATGCTGCCCAGCAAGGCTGGCATCTGGAGGGT
GGAGTGCCTGATTGGGGAGCACCTGCATGCTGGCATGAGCACCCTGTTCCTGGTGTACAGCAACAAGT
GCCAGACCCCCCTGGGCATGGCCTCTGGCCACATCAGGGACTTCCAGATCACTGCCTCTGGCCAGTAT
GGCCAGTGGGCCCCCAAGCTGGCCAGGCTGCACTACTCTGGCAGCATCAATGCCTGGAGCACCAAGGA
GCCCTTCAGCTGGATCAAGGTGGACCTGCTGGCCCCCATGATCATCCATGGCATCAAGACCCAGGGGG
CCAGGCAGAAGTTCAGCAGCCTGTACATCAGCCAGTTCATCATCATGTACAGCCTGGATGGCAAGAAG
TGGCAGACCTACAGGGGCAACAGCACTGGCACCCTGATGGTGTTCTTTGGCAATGTGGACAGCTCTGG
CATCAAGCACAACATCTTCAACCCCCCCATCATTGCCAGATACATCAGGCTGCACCCCACCCACTACA
GCATCAGGAGCACCCTGAGGATGGAGCTGATGGGCTGTGACCTGAACAGCTGCAGCATGCCCCTGGGC
ATGGAGAGCAAGGCCATCTCTGATGCCCAGATCACTGCCAGCAGCTACTTCACCAACATGTTTGCCAC
CTGGAGCCCCAGCAAGGCCAGGCTGCACCTGCAGGGCAGGAGCAATGCCTGGAGGCCCCAGGTCAACA
ACCCCAAGGAGTGGCTGCAGGTGGACTTCCAGAAGACCATGAAGGTGACTGGGGTGACCACCCAGGGG
GTGAAGAGCCTGCTGACCAGCATGTATGTGAAGGAGTTCCTGATCAGCAGCAGCCAGGATGGCCACCA
GTGGACCCTGTTCTTCCAGAATGGCAAGGTGAAGGTGTTCCAGGGCAACCAGGACAGCTTCACCCCTG
TGGTGAACAGCCTGGACCCCCCCCTGCTGACCAGATACCTGAGGATTCACCCCCAGAGCTGGGTGCAC
CAGATTGCCCTGAGGATGGAGGTGCTGGGCTGTGAGGCCCAGGACCTGTACTGATTAATTAAGAGCAT
CTTACCGCCATTTATTCCCATATTTGTTCTGTTTTTCTTGATTTGGGTATACATTTAAATGTTAATAA
AACAAAATGGTGGGGCAATCATTTACATTTTTAGGGATATGTAATTACTAGTTCAGGTGTATTGCCAC
AAGACAAACATGTTAAGAAACTTTCCCGTTATTTACGCTCTGTTCCTGTTAATCAACCTCTGGATTAC
AAAATTTGTGAAAGATTGACTGATATTCTTAACTATGTTGCTCCTTTTACGCTGTGTGGATATGCTGC
TTTATAGCCTCTGTATCTAGCTATTGCTTCCCGTACGGCTTTCGTTTTCTCCTCCTTGTATAAATCCT
GGTTGCTGTCTCTTTTAGAGGAGTTGTGGCCCGTTGTCCGTCAACGTGGCGTGGTGTGCTCTGTGTTT
GCTGACGCAACCCCCACTGGCTGGGGCATTGCCACCACCTGTCAACTCCTTTCTGGGACTTTCGCTTT
CCCCCTCCCGATCGCCACGGCAGAACTCATCGCCGCCTGCCTTGCCCGCTGCTGGACAGGGGCTAGGT
TGCTGGGCACTGATAATTCCGTGGTGTTGTCTGTGCCTTCTAGTTGCCAGCCATCTGTTGTTTGCCCC
TCCCCCGTGCCTTCCTTGACCCTGGAAGGTGCCACTCCCACTGTCCTTTCCTAATAAAATGAGGAAAT
TGCATCGCATTGTCTGAGTAGGTGTCATTCTATTCTGGGGGGTGGGGTGGGGCAGGACAGCAAGGGGG
AGGATTGGGAAGACAATAGCAGGCATGCTGGGGATGCGGTGGGCTCTATGGCTCTAGAGCATGGCTAC
GTAGATAAGTAGCATGGCGGGTTAATCATTAACTACACCTGCAGGAGGAACCCCTAGTGATGGAGTTG
GCCACTCCCTCTCTGCGCGCTCGCTCGCTCACTGAGGGAGGCCGCCCGGGCAAAGCCCGGGCGTCGGG
CGACCTTTGGTCGCCCGGCCTCCCTC
073 (Lane 5; 6215 bp; hFVIII ORF 993-5384)
GAGGCCGCCCGGGCAAAGCCCGGGCGTCGGGCGACCTTTGGTCGCCCGGCCTCCCTCCCTCAGTGAGC
GAGCGAGCGCGCAGAGAGGGAGTGGCCAACTCCATCACTAGGGGTTCCTCCTGCAGGTGTAGTTAATG
ATTAACCCGCCATGCTACTTATCTACGTAGCCATGCTCTAGAGCCATAGAGCCCACCGCATCCCCAGC
ATGCCTGCTATTGTCTTCCCAATCCTCCCCCTTGCTGTCCTGCCCCACCCCACCCCCCAGAATAGAAT
GACACCTACTCAGACAATGCGATGCAATTTCCTCATTTTATTAGGAAAGGACAGTGGGAGTGGCACCT
TCCAGGGTCAAGGAAGGCACGGGGGAGGGGCAAACAACAGATGGCTGGCAACTAGAAGGCACAGACAA
CACCACGGAATTATCAGTGCCCAGCAACCTAGCCCCTGTCCAGCAGCGGGCAAGGCAGGCGGCGATGA
GTTCTGCCGTGGCGATCGGGAGGGGGAAAGCGAAAGTCCCAGAAAGGAGTTGACAGGTGGTGGCAATG
CCCCAGCCAGTGGGGGTTGCGTCAGCAAACACAGAGCACACCACGCCACGTTGACGGACAACGGGCCA
CAACTCCTCTAAAAGAGACAGCAACCAGGATTTATACAAGGAGGAGAAAACGAAAGCCGTACGGGAAG
CAATAGCTAGATACAGAGGCTATAAAGCAGCATATCCACACAGCGTAAAAGGAGCAACATAGTTAAGA
ATATCAGTCAATCTTTCACAAATTTTGTAATCCAGAGGTTGATTAACAGGAACAGAGCGTAAATAACG
GGAAAGTTTCTTAACATGTTTGTCTTGTGGCAATACACCTGAACTAGTAATTACATATCCCTAAAAAT
GTAAATGATTGCCCCACCATTTTGTTTTATTAACATTTAAATGTATACCCAAATCAAGAAAAACAGAA
CAAATATGGGAATAAATGGCGGTAAGATGCTCTTAATTAATCAGTACAGGTCCTGGGCCTCACAGCCC
AGCACCTCCATCCTCAGGGCAATCTGGTGCACCCAGCTCTGGGGGTGAATCCTCAGGTATCTGGTCAG
CAGGGGGGGGTCCAGGCTGTTCACCACAGGGGTGAAGCTGTCCTGGTTGCCCTGGAACACCTTCACCT
TGCCATTCTGGAAGAACAGGGTCCACTGGTGGCCATCCTGGCTGCTGCTGATCAGGAACTCCTTCACA
TACATGCTGGTCAGCAGGCTCTTCACCCCCTGGGTGGTCACCCCAGTCACCTTCATGGTCTTCTGGAA
GTCCACCTGCAGCCACTCCTTGGGGTTGTTGACCTGGGGCCTCCAGGCATTGCTCCTGCCCTGCAGGT
GCAGCCTGGCCTTGCTGGGGCTCCAGGTGGCAAACATGTTGGTGAAGTAGCTGCTGGCAGTGATCTGG
GCATCAGAGATGGCCTTGCTCTCCATGCCCAGGGGCATGCTGCAGCTGTTCAGGTCACAGCCCATCAG
CTCCATCCTCAGGGTGCTCCTGATGCTGTAGTGGGTGGGGTGCAGCCTGATGTATCTGGCAATGATGG
GGGGGTTGAAGATGTTGTGCTTGATGCCAGAGCTGTCCACATTGCCAAAGAACACCATCAGGGTGCCA
GTGCTGTTGCCCCTGTAGGTCTGCCACTTCTTGCCATCCAGGCTGTACATGATGATGAACTGGCTGAT
GTACAGGCTGCTGAACTTCTGCCTGGCCCCCTGGGTCTTGATGCCATGGATGATCATGGGGGCCAGCA
GGTCCACCTTGATCCAGCTGAAGGGCTCCTTGGTGCTCCAGGCATTGATGCTGCCAGAGTAGTGCAGC
CTGGCCAGCTTGGGGGCCCACTGGCCATACTGGCCAGAGGCAGTGATCTGGAAGTCCCTGATGTGGCC
AGAGGCCATGCCCAGGGGGGTCTGGCACTTGTTGCTGTACACCAGGAACAGGGTGCTCATGCCAGCAT
GCAGGTGCTCCCCAATCAGGCACTCCACCCTCCAGATGCCAGCCTTGCTGGGCAGCATCTCCACAGTC
TCAAACACCCCAGGGTACAGGTTGTACAGGGCCATCTTGTACTCCTCCTTCTTCCTCACAGTGAACAC
ATGGCCAGAGAAGTGGATGCTGTGGATGTTCTCATTGCTGCCCATGCTCAGCAGGTACCACCTGATCC
TCTGGTCCTGGGCCATCACCAGGCCAGGCAGGGTGTCCATGATGTAGCCATTGATGGCATGGAACCTG
TAGTTCTCCTTGAAGGTGGGGTCCTCCATCTGGATGTTGCAGGGGGCCCTGCAGTTCCTCTCCATGTT
CTCAGTGAAGTACCAGCTCTTGGTTTCATCAAAGATGGTGAAGAACAGGGCAAACTCCTGCACAGTCA
CCTGCCTGCCATGGGCAGGGTTCAGGGTGTTGGTGTGGCACACCAGCAGGGGGCCAATCAGGCCAGAG
TGCACATCCTTCTCCAGGTCCACATCAGAGAAGTAGGCCCAGGCCTTGCAGTCAAACTCATCCTTGGT
GGGGGCCATGTGGTGCTGCACCTTCCAGAAGTAGGTCTTGGTTTCATTGGGCTTCACAAAGTTCTTCC
TGGGCTCAGCCCCCTGCCTCTGGTCCTCCTCATAGCTGATCAGGCTGCTGTAGAAGCTGTAGGGCCTG
CTGGCCTGGTTCCTGAAGGTCACCATGATGTTGTCCTCCACCTCAGCCCTGATGTAGGGGCCCAGCAG
GCCCAGGTGCTCATTCAGCTCCCCTCTGTACAGGGGCTGGGTGAAGCTGCCATCAGTGAACTCCTGGA
ACACCACCTTCTTGAACTGGGGCACAGAGCCAGACTGGGCCCTGTTCCTCAGCACATGGGGGCTGCTG
CTCATGCCATAGTCCCACAGCCTCTCCACAGCAGCAATGAAGTAGTGCCTGGTCTTCTTCTGGAAGCT
CCTGGGGCTCTGGTTCTCGTCCTCGTCGTAGATGTCAAAGTCCTCCTTCTTCATCTCCACAGAGATGG
TGTCATCATAGTCAATCTCCTCCTGGTCAGACTGCAGGGTGGTATTCTCTGGGATGGTGGTGGCATTG
AACTGCTTCTGCCTGGTGCTGGGGTGCCTGCTATTCTGGCTGAAGCTCCTGGGCTCAATGGCATTGTT
CTTGCTCAGCAGGTAGGCAGAGATGTCCTCATAGCTGTCCTCATAGTAGTCCCCAGTGTTCTTGTCAC
AGCTGGAGACTTTCAGCAGGGCAGTCATGCCCCTGTTCCTGAAGTCAGAGTTGTGGCAGCCCAGAATC
CACAGGCCAGGGTTCTCCATGCTCATGAACACAGTCTCCCCAGAGAAGGGGAACAGGGTCAGGGTGTC
CTCATACACCATCTTGTGCTTGAAGGTGTAGCCAGAGAAGAACACAGACAGGAAGTCAGTCTGGGCCC
CAATGCTCAGGATGTACCAGTAGGCCACCTCATGCAGGCACACAGACAGCTGCAGGCTGTCAAACACA
TAGCCATTGATGCTGTGCATGATGTTGCTGGCCTGGAACTCAGGGTCCTCCAGCTGCACCCCAGCAGG
GTTGGGCAGGAACCTCTGGATGTTCTCAGTCAGGTACCAGCTCCTGTTCTCATCAAACACAGAGAACA
GGATCACATTCCTCTTGTCAGACATGATCTGGTTGCCCCTCTGGTCCACAGACTCCTTGTAGCAGATC
AGCAGGGGGCCAATCAGGCCAGAGGCCAGGTCCCTCTCCATGTTCACAAAGCTGCTGTAGTATCTGGT
CAGGCACCTGGGGTCAGACTTGGTGGGGCCATCCTCCACAGTCACAGTCCACTTGTACTTGAAGATCT
CCCCAGGCAGGATGGGGAAGTCCTTCAGGTGCTTCACCCCCTTGGGCAGCCTCCTGCTGTACAGGGGC
CTCACATCAGTGATGCCATGGGGGTAGATGTTGTAGGGCCTGCTGGCCTGGTTCTTGAAGATGATCAG
CAGGGTGTCCCCCACCTCCCCATACAGCAGGGGGCCCAGGATGCCAGACTCATGCTGGATGGCCTCCC
TGGTCTTGAAGGTTTCATCAGTGTAGGCCATGAACCTGACCTTCTTGTACTTCCTGCCAATCCTCTGG
GGGCCATTGTTCAGGTACTGGCTCTTGTAGCTCCTGTCATCAGGGGCCAGCACCAGGGGGGCATAGTC
CCAGTCCTCCTCCTCAGCAGCAATGTAGTGCACCCAGGTCTTGGGGTGCTTCTTGGCCACAGACCTGA
TCTGGATGAAGCTGGGGCTGTTGTCATCATCAAACCTCACCACATCCATCTCAGAGTCAGTCAGGTCA
TCATCATAGTCCTCAGCCTCCTCATTGTTCTTCATCCTCAGCTGGGGCTCCTCAGGGCAGCTGTCCAC
CTTCACATAGGCCTCCATGCCATCATGCTGGTGGCTGCTGATGTGGCAGAACAGCAGGAACTGGCCCA
GGTCCATCAGCAGGGTCTGGGCAGTCAGGAAGGTGATGGGGCTGATCTCCAGGCTGGCCTGCCTGTGG
TTCCTGACCAGGAAGGTGTGGCCCTCCAGGAAGATGCTGTGCACCTCAGGGGTGGTGCCCATGCCAAT
CACATGCCAGTACACAGACTTCCTGTGGCAGCCAATCAGGCCAGGCAGGCTCCTGTTCACATAGCCAT
TCACAGTGTGCATCTTGGGCCAGGCCCTGGCAGAGGCAGCATCCCTGTCCTGCATCAGGCTGTTCTTG
GTTTCAGAGTGCCAGCTCTTGCCCTCATCAAACACAGCAAACAGCAGGATGAACTTGTGCAGGGTCTG
GGTCTTCTCCTTGGCCAGGCTGCCCTCCCTGCACACCAGCAGGGCCCCAATCAGGCCAGAGTTCAGGT
CCTTCACCAGGTCCACATGGCTCAGGTAGCTGTAGGTCAGGCACAGGGGGTCAGAGGCCATGGGGCCA
TTCTCCTTCAGCACCTGCCACACATAGGTGTGGCTGCCCCCAGGGAACACCTTGTCATCCTCCTTCTC
CCTCTGGCTGGTCTGGTCATCATACTCAGCCCCCTCAGAGGCCTTCCAGTAGCTCACCCCCACAGCAT
GCAGGCTCACAGGGTGGCTGGCCATGTTCTTCAGGGTGATCACCACAGTGTCATACACCTCAGCCTGG
ATGGTGGGGCCCAGCAGGCCCATCCAGGGGGGCCTGGGCTTGGCAATGTTGAACAGGTGGTCAGTGAA
CTCCACAAACAGGGTCTTCTTGTACACCACAGAGGTGTTGAAGGGGAAGCTCTTGGGCACTCTGGGGG
GGAACCTGGCATCCACAGGCAGCTCCCCCAGGTCAGACTGCATGTAGTCCCAGCTCAGCTCCACAGCC
CCCAGGTAGTATCTCCTGGTGGCAGAGAAGCAGAACCTCAGCAGGCACAGGAAGAAGCAGGTGCTCAG
CTCAATCTGCATGGTGGCGGCGTTTAAACCAACCTGAAAAAAAGTGATTTCAGGCAGGTGCTCCAGGT
AATTAAACATTAATACCCCACCAACCAACCATCCCTTAAACCCTTACCTCTTCAGGAGCTTGTGGATC
TGTGTGACGGCTTCTCCTGGTGAAGGGGCTTTTATACCCCCTCCTTCCAACCCAGGCTGCTGATCCCT
GCCAAGCTGACTCCAAACCTGCTGATTCTGATTATTGACTTAGTCAACAAAAGGAGAATAAGTAACCT
ACACAAATATGAACCTTGCCTAGGGAGATTAGAGTATCGGAACACTCGCTCTACGAAATGTGCAGACA
GACGGGGATCCTCTAGAGCTACCTGCTGATCGCCCGGCCCCTGTTCAAACATGTCCTAATACTCTGTC
TCTGCAAGGGTCATCAGTAGTTTTCCATCTTACTCAACATCCTCCCAGTGGGTACCGTGGACTTAGCC
CCTGTTTGCTCCTCCGATAACTGGGGTGACCTTGGTTAATATTCACCAGCAGCCTCCCCCGGTGGACT
TAGCCCCTGTTTGCTCCTCCGATAACTGGGGTGACCTTGGTTAATATTCACCAGCAGCCTCCCCCGGT
GGACTTAGCCCCTGTTTGCTCCTCCGATAACTGGGGTGACCTTGGTTAATATTCACCAGCAGCCTCCC
CCGCGGCCGCGATAAGTAGCATGGCGGGTTAATCATTAACTACAAGGAACCCCTAGTGATGGAGCCTC
AGCTTGGCCACTCCCTCTCTGCGCGCTCGCTCGCTCACTGAGGCCGCCCGGGCAAAGCCCGGGCGTCG
GGCGACCTTTGGTCGCCCGGCCTCAIT
706 (Lanes 6-7; 3605 bp; Luciferase)
CTGAGGCCGCCCGGGCAAAGCCCGGGCGTCGGGCGACCTTTGGTCGCCCGGCCTCANTGAGCTAGTGA
GCTAGCAGAGAGGGAGTGGCCAAGCTGAGGCTCCATCACTAGGGGTTCCTTGTAGTTAATGATTAACC
CACCATGCTACTTATGGCTAGGACTAGACTAGTAGGCTCAGAGGCACACAGGAGTTTCTGGGCTCACC
CTGCCCCCTTCCAACCCCTCAGTTCCCATCCTCCAGCAGCTGTTTGTGTGCTGCCTCTGAAGTCCACA
CTGAACAAACTTCAGCCTACTCATGTCCCTAAAATGGGCAAACATTGCAAGCAGCAAACAGCAAACAC
ACAGCCCTCCCTGCCTGCTGACCTTGGAGCTGGGGCAGAGGTCAGTGACCTCTCTGGGCCCATGCCAC
CTCCAACATCCACTTGACCCCTTGGAATTTCAGTGGAGAGGAGCAGAGGTTGTCCTGGTGTGGTTTAG
GTAGTGTGAGAGGGTCCAGGTTCAAAACCACTTGCTGGGTGGGGAGTTGTCAGTAAGTGGCTATGCCC
CAACCCTGAAGCCTGTTTCCCCATCTGTACAATGGAAATGATAAAGACCCCCATCTGATAGGGTTTTT
GTGGCAAATAAACATTTGGTTTTTTTGTTTTGTTTTGTTTTGTTTTTTGAGATGGAGGTTTGCTCTGT
GGCCCAGGCTGGAGTGCAGTGACACAATCTCATCTCACCACAACCTTCCCCTGCCTCATCCTCCCAAG
TAGCTGGGATTACAAGCATGTGCCACCACACCTGGCTAATTTTCTATTTTTAGTAGAGATGGGTTTCT
CCATGTTGGTCAGGCTCAGCCTCCCAAGTAACTGGGATTACAGGCCTGTGCCACCACACCTGGCTAAT
TTTTTCTATTTTTGACAGGGATGGGGTTTCACCATGTTGGTCAGGCTGGTCTAGAGGTACTGGATCTT
GCTACCAGTGGAACAGCCACTAAGGATTCTGCAGTGAGAGCAGAGGGCCAGCTAAGTGGTACTCTCCC
AGAGACTGTCTGACTCATGCCACCCCCTCCACCTTGGACACAGGACACTGTGGTTTCTGAGCCAGGTA
CAATGACTCCTTTTGGTAAGTGCAGTGGAAGCTGTACACTGCCCAGGCAAAGTGTCTGGGCAGCATAG
GCAGGTGACTCAGATCCCAGCCAGTGGACTTAGCCCCTGTTTGCTCCTCTGATAACTGGGGTGACCTT
GGTTAATATTCACCAGCAGCCTCCCCTGTTGCCCCTCTGGATCCACTGCTTAAATACAGACAAGGACA
GGGCCCTGTCTCTTCAGCTTCAGGCACCACCACTGACCTGGGACAGTGAATAATTACTCTAAGGTAAA
TATAAAATTTTTAAGTGTATAATGTGTTAAACTACTGATTCTAATTGTTTCTCTCTTTTAGATTCCAA
CCTTTGGAACTGAACCATGGAGGATGCCAAGAACATTAAGAAAGGCCCTGCCCCCTTCTACCCCCTGG
AGGATGGAACAGCAGGGGAGCAGCTGCACAAGGCCATGAAGAGATATGCCCTGGTCCCAGGCACCATT
GCCTTCACTGATGCCCACATTGAAGTTGATATCACCTATGCTGAATATTTTGAGATGTCTGTGAGACT
GGCAGAGGCTATGAAAAGGTATGGACTGAACACCAACCACAGAATTGTGGTGTGCAGTGAAAATTCCC
TGCAGTTCTTCATGCCTGTGCTTGGAGCTCTCTTCATTGGAGTAGCTGTTGCTCCAGCCAATGACATC
TACAATGAAAGAGAGCTCCTCAACTCCATGGGCATCTCCCAGCCCACTGTGGTGTTTGTGTCCAAAAA
GGGGCTGCAGAAAATTCTGAATGTGCAGAAGAAGCTGCCAATCATCCAGAAGATCATTATCATGGACA
GCAAGACAGATTACCAGGGTTTCCAGAGCATGTACACCTTTGTCACCAGCCACCTCCCTCCTGGCTTC
AATGAGTATGATTTTGTTCCAGAGAGCTTTGACAGAGATAAAACAATTGCACTGATTATGAACAGCTC
TGGCAGCACAGGTCTGCCCAAAGGTGTGGCCTTGCCCCACAGGACTGCCTGTGTCAGGTTCTCTCATG
CCAGGGACCCCATCTTTGGAAACCAGATCATCCCTGATACAGCCATCCTGTCTGTTGTGCCTTTCCAT
CATGGCTTTGGCATGTTCACCACCCTGGGCTACCTGATCTGTGGATTCAGAGTAGTGCTGATGTATAG
GTTTGAGGAGGAACTGTTCCTGAGGAGCCTTCAGGACTACAAGATCCAATCTGCTCTGCTGGTGCCCA
CCCTCTTTTCCTTCTTTGCCAAGAGCACCTTGATTGATAAGTATGACCTGAGCAACCTGCATGAAATT
GCTTCTGGAGGAGCCCCTCTGTCCAAGGAAGTGGGAGAGGCAGTGGCCAAAAGATTCCACCTGCCTGG
AATCAGACAGGGCTATGGCCTGACAGAGACAACTTCTGCCATTCTCATCACTCCAGAAGGAGATGACA
AGCCAGGAGCAGTGGGCAAAGTGGTGCCATTTTTTGAAGCCAAGGTGGTGGATCTGGACACAGGCAAG
ACTCTGGGAGTGAATCAGAGAGGTGAGCTGTGTGTGAGGGGCCCCATGATCATGTCAGGATATGTGAA
CAACCCTGAGGCCACCAATGCCCTCATTGACAAAGATGGCTGGCTGCACAGTGGAGACATTGCCTACT
GGGATGAAGATGAGCACTTCTTCATAGTGGACAGGCTGAAGTCCCTCATCAAATACAAAGGGTACCAA
GTGGCTCCTGCTGAGCTGGAGTCCATCCTGCTCCAGCACCCCAACATCTTTGATGCAGGAGTGGCAGG
CCTCCCAGATGATGATGCTGGAGAACTTCCAGCTGCTGTGGTTGTCCTGGAACATGGAAAGACCATGA
CTGAGAAGGAGATTGTTGACTATGTTGCCAGCCAGGTGACCACAGCTAAGAAGCTCAGAGGAGGGGTG
GTCTTTGTAGATGAGGTGCCCAAGGGCCTGACTGGAAAACTGGATGCCAGAAAAATCAGGGAGATCTT
GATCAAGGCAAAGAAAGGAGGGAAGATTGCTGTGTGATTAATTAAGCTTGCTTTCTTGCTGTCCAATT
TCTATTAAAGGTTCCTTTGTTCCCTAAGTCCAACTACTAAACTGGGGGATATTATGAAGGGCCTTGAG
CATCTGGATTCTGCCTCAGACATGATAAGATACATTGATGAGTTTGGACAAACCACAACTAGAATGCA
GTGAAAAAAATGCTTTATTTGTGAAATTTGTGATGCTATTGCTTTATTTGTAACCATTATAAGCTGCA
ATAAACAAGTTAACAACAACAATTGCATTCATTTTATGTTTCAGGTTCAGGGGGAGATGTGGGAGGTT
TTTTAAAGCAAGTAAAACCTCTACAAATGTGGTACTTAAGCCACAATCTGCCTCCCAGTAGTACATGA
CATTAGTTTATTAATAGCCTAGGAACCCCTAGTGATGGAGTTGGCCACTCCCTCTCTGCTAGCTCACT
AGCTCACTGAGGGAGGCCGCCCGGGCAAAGCCCGGGCGTCGGGCGACCTTTGGTCGCCCGGCCTCCCT
C
075 (Lane 7; 3606 bp; Luciferase)
GAGGCCGCCCGGGCAAAGCCCGGGCGTCGGGCGACCTTTGGTCGCCCGGCCTCCCTCCCTCAGTGAGC
TAGTGAGCTAGCAGAGAGGGAGTGGCCAACTCCATCACTAGGGGTTCCTAGGCTATTAATAAACTAAT
GTCATGTACTACTGGGAGGCAGATTGTGGCTTAAGTACCACATTTGTAGAGGTTTTACTTGCTTTAAA
AAACCTCCCACATCTCCCCCTGAACCTGAAACATAAAATGAATGCAATTGTTGTTGTTAACTTGTTTA
TTGCAGCTTATAATGGTTACAAATAAAGCAATAGCATCACAAATTTCACAAATAAAGCATTTTTTTCA
CTGCATTCTAGTTGTGGTTTGTCCAAACTCATCAATGTATCTTATCATGTCTGAGGCAGAATCCAGAT
GCTCAAGGCCCTTCATAATATCCCCCAGTTTAGTAGTTGGACTTAGGGAACAAAGGAACCTTTAATAG
AAATTGGACAGCAAGAAAGCAAGCTTAATTAATCACACAGCAATCTTCCCTCCTTTCTTTGCCTTGAT
CAAGATCTCCCTGATTTTTCTGGCATCCAGTTTTCCAGTCAGGCCCTTGGGCACCTCATCTACAAAGA
CCACCCCTCCTCTGAGCTTCTTAGCTGTGGTCACCTGGCTGGCAACATAGTCAACAATCTCCTTCTCA
GTCATGGTCTTTCCATGTTCCAGGACAACCACAGCAGCTGGAAGTTCTCCAGCATCATCATCTGGGAG
GCCTGCCACTCCTGCATCAAAGATGTTGGGGTGCTGGAGCAGGATGGACTCCAGCTCAGCAGGAGCCA
CTTGGTACCCTTTGTATTTGATGAGGGACTTCAGCCTGTCCACTATGAAGAAGTGCTCATCTTCATCC
CAGTAGGCAATGTCTCCACTGTGCAGCCAGCCATCTTTGTCAATGAGGGCATTGGTGGCCTCAGGGTT
GTTCACATATCCTGACATGATCATGGGGCCCCTCACACACAGCTCACCTCTCTGATTCACTCCCAGAG
TCTTGCCTGTGTCCAGATCCACCACCTTGGCTTCAAAAAATGGCACCACTTTGCCCACTGCTCCTGGC
TTGTCATCTCCTTCTGGAGTGATGAGAATGGCAGAAGTTGTCTCTGTCAGGCCATAGCCCTGTCTGAT
TCCAGGCAGGTGGAATCTTTTGGCCACTGCCTCTCCCACTTCCTTGGACAGAGGGGCTCCTCCAGAAG
CAATTTCATGCAGGTTGCTCAGGTCATACTTATCAATCAAGGTGCTCTTGGCAAAGAAGGAAAAGAGG
GTGGGCACCAGCAGAGCAGATTGGATCTTGTAGTCCTGAAGGCTCCTCAGGAACAGTTCCTCCTCAAA
CCTATACATCAGCACTACTCTGAATCCACAGATCAGGTAGCCCAGGGTGGTGAACATGCCAAAGCCAT
GATGGAAAGGCACAACAGACAGGATGGCTGTATCAGGGATGATCTGGTTTCCAAAGATGGGGTCCCTG
GCATGAGAGAACCTGACACAGGCAGTCCTGTGGGGCAAGGCCACACCTTTGGGCAGACCTGTGCTGCC
AGAGCTGTTCATAATCAGTGCAATTGTTTTATCTCTGTCAAAGCTCTCTGGAACAAAATCATACTCAT
TGAAGCCAGGAGGGAGGTGGCTGGTGACAAAGGTGTACATGCTCTGGAAACCCTGGTAATCTGTCTTG
CTGTCCATGATAATGATCTTCTGGATGATTGGCAGCTTCTTCTGCACATTCAGAATTTTCTGCAGCCC
CTTTTTGGACACAAACACCACAGTGGGCTGGGAGATGCCCATGGAGTTGAGGAGCTCTCTTTCATTGT
AGATGTCATTGGCTGGAGCAACAGCTACTCCAATGAAGAGAGCTCCAAGCACAGGCATGAAGAACTGC
AGGGAATTTTCACTGCACACCACAATTCTGTGGTTGGTGTTCAGTCCATACCTTTTCATAGCCTCTGC
CAGTCTCACAGACATCTCAAAATATTCAGCATAGGTGATATCAACTTCAATGTGGGCATCAGTGAAGG
CAATGGTGCCTGGGACCAGGGCATATCTCTTCATGGCCTTGTGCAGCTGCTCCCCTGCTGTTCCATCC
TCCAGGGGGTAGAAGGGGGCAGGGCCTTTCTTAATGTTCTTGGCATCCTCCATGGTTCAGTTCCAAAG
GTTGGAATCTAAAAGAGAGAAACAATTAGAATCAGTAGTTTAACACATTATACACTTAAAAATTTTAT
ATTTACCTTAGAGTAATTATTCACTGTCCCAGGTCAGTGGTGGTGCCTGAAGCTGAAGAGACAGGGCC
CTGTCCTTGTCTGTATTTAAGCAGTGGATCCAGAGGGGCAACAGGGGAGGCTGCTGGTGAATATTAAC
CAAGGTCACCCCAGTTATCAGAGGAGCAAACAGGGGCTAAGTCCACTGGCTGGGATCTGAGTCACCTG
CCTATGCTGCCCAGACACTTTGCCTGGGCAGTGTACAGCTTCCACTGCACTTACCAAAAGGAGTCATT
GTACCTGGCTCAGAAACCACAGTGTCCTGTGTCCAAGGTGGAGGGGGTGGCATGAGTCAGACAGTCTC
TGGGAGAGTACCACTTAGCTGGCCCTCTGCTCTCACTGCAGAATCCTTAGTGGCTGTTCCACTGGTAG
CAAGATCCAGTACCTCTAGACCAGCCTGACCAACATGGTGAAACCCCATCCCTGTCAAAAATAGAAAA
AATTAGCCAGGTGTGGTGGCACAGGCCTGTAATCCCAGTTACTTGGGAGGCTGAGCCTGACCAACATG
GAGAAACCCATCTCTACTAAAAATAGAAAATTAGCCAGGTGTGGTGGCACATGCTTGTAATCCCAGCT
ACTTGGGAGGATGAGGCAGGGGAAGGTTGTGGTGAGATGAGATTGTGTCACTGCACTCCAGCCTGGGC
CACAGAGCAAACCTCCATCTCAAAAAACAAAACAAAACAAAACAAAAAAACCAAATGTTTATTTGCCA
CAAAAACCCTATCAGATGGGGGTCTTTATCATTTCCATTGTACAGATGGGGAAACAGGCTTCAGGGTT
GGGGCATAGCCACTTACTGACAACTCCCCACCCAGCAAGTGGTTTTGAACCTGGACCCTCTCACACTA
CCTAAACCACACCAGGACAACCTCTGCTCCTCTCCACTGAAATTCCAAGGGGTCAAGTGGATGTTGGA
GGTGGCATGGGCCCAGAGAGGTCACTGACCTCTGCCCCAGCTCCAAGGTCAGCAGGCAGGGAGGGCTG
TGTGTTTGCTGTTTGCTGCTTGCAATGTTTGCCCATTTTAGGGACATGAGTAGGCTGAAGTTTGTTCA
GTGTGGACTTCAGAGGCAGCACACAAACAGCTGCTGGAGGATGGGAACTGAGGGGTTGGAAGGGGGCA
GGGTGAGCCCAGAAACTCCTGTGTGCCTCTGAGCCTACTAGTCTAGTCCTAGCCATAAGTAGCATGGT
GGGTTAATCATTAACTACAAGGAACCCCTAGTGATGGAGCCTCAGCTTGGCCACTCCCTCTCTGCTAG
CTCACTAGCTCACTGAGGCCGCCCGGGCAAAGCCCGGGCGTCGGGCGACCTTTGGTCGCCCGGCCTCA
IT
707 (Lanes 8-9; 6214 bp; hFVIII ORF 831-5222)
CTGAGGCCGCCCGGGCAAAGCCCGGGCGTCGGGCGACCTTTGGTCGCCCGGCCTCANTGAGCGAGCGA
GCGCGCAGAGAGGGAGTGGCCAAGCTGAGGCTCCATCACTAGGGGTTCCTTGTAGTTAATGATTAACC
CGCCATGCTACTTATCGCGGCCGCGGGGGAGGCTGCTGGTGAATATTAACCAAGGTCACCCCAGTTAT
CGGAGGAGCAAACAGGGGCTAAGTCCACCGGGGGAGGCTGCTGGTGAATATTAACCAAGGTCACCCCA
GTTATCGGAGGAGCAAACAGGGGCTAAGTCCACCGGGGGAGGCTGCTGGTGAATATTAACCAAGGTCA
CCCCAGTTATCGGAGGAGCAAACAGGGGCTAAGTCCACGGTACCCACTGGGAGGATGTTGAGTAAGAT
GGAAAACTACTGATGACCCTTGCAGAGACAGAGTATTAGGACATGTTTGAACAGGGGCCGGGCGATCA
GCAGGTAGCTCTAGAGGATCCCCGTCTGTCTGCACATTTCGTAGAGCGAGTGTTCCGATACTCTAATC
TCCCTAGGCAAGGTTCATATTTGTGTAGGTTACTTATTCTCCTTTTGTTGACTAAGTCAATAATCAGA
ATCAGCAGGTTTGGAGTCAGCTTGGCAGGGATCAGCAGCCTGGGTTGGAAGGAGGGGGTATAAAAGCC
CCTTCACCAGGAGAAGCCGTCACACAGATCCACAAGCTCCTGAAGAGGTAAGGGTTTAAGGGATGGTT
GGTTGGTGGGGTATTAATGTTTAATTACCTGGAGCACCTGCCTGAAATCACTTTTTTTCAGGTTGGTT
TAAACGCCGCCACCATGCAGATTGAGCTGAGCACCTGCTTCTTCCTGTGCCTGCTGAGGTTCTGCTTC
TCTGCCACCAGGAGATACTACCTGGGGGCTGTGGAGCTGAGCTGGGACTACATGCAGTCTGACCTGGG
GGAGCTGCCTGTGGATGCCAGGTTCCCCCCCAGAGTGCCCAAGAGCTTCCCCTTCAACACCTCTGTGG
TGTACAAGAAGACCCTGTTTGTGGAGTTCACTGACCACCTGTTCAACATTGCCAAGCCCAGGCCCCCC
TGGATGGGCCTGCTGGGCCCCACCATCCAGGCTGAGGTGTATGACACTGTGGTGATCACCCTGAAGAA
CATGGCCAGCCACCCTGTGAGCCTGCATGCTGTGGGGGTGAGCTACTGGAAGGCCTCTGAGGGGGCTG
AGTATGATGACCAGACCAGCCAGAGGGAGAAGGAGGATGACAAGGTGTTCCCTGGGGGCAGCCACACC
TATGTGTGGCAGGTGCTGAAGGAGAATGGCCCCATGGCCTCTGACCCCCTGTGCCTGACCTACAGCTA
CCTGAGCCATGTGGACCTGGTGAAGGACCTGAACTCTGGCCTGATTGGGGCCCTGCTGGTGTGCAGGG
AGGGCAGCCTGGCCAAGGAGAAGACCCAGACCCTGCACAAGTTCATCCTGCTGTTTGCTGTGTTTGAT
GAGGGCAAGAGCTGGCACTCTGAAACCAAGAACAGCCTGATGCAGGACAGGGATGCTGCCTCTGCCAG
GGCCTGGCCCAAGATGCACACTGTGAATGGCTATGTGAACAGGAGCCTGCCTGGCCTGATTGGCTGCC
ACAGGAAGTCTGTGTACTGGCATGTGATTGGCATGGGCACCACCCCTGAGGTGCACAGCATCTTCCTG
GAGGGCCACACCTTCCTGGTCAGGAACCACAGGCAGGCCAGCCTGGAGATCAGCCCCATCACCTTCCT
GACTGCCCAGACCCTGCTGATGGACCTGGGCCAGTTCCTGCTGTTCTGCCACATCAGCAGCCACCAGC
ATGATGGCATGGAGGCCTATGTGAAGGTGGACAGCTGCCCTGAGGAGCCCCAGCTGAGGATGAAGAAC
AATGAGGAGGCTGAGGACTATGATGATGACCTGACTGACTCTGAGATGGATGTGGTGAGGTTTGATGA
TGACAACAGCCCCAGCTTCATCCAGATCAGGTCTGTGGCCAAGAAGCACCCCAAGACCTGGGTGCACT
ACATTGCTGCTGAGGAGGAGGACTGGGACTATGCCCCCCTGGTGCTGGCCCCTGATGACAGGAGCTAC
AAGAGCCAGTACCTGAACAATGGCCCCCAGAGGATTGGCAGGAAGTACAAGAAGGTCAGGTTCATGGC
CTACACTGATGAAACCTTCAAGACCAGGGAGGCCATCCAGCATGAGTCTGGCATCCTGGGCCCCCTGC
TGTATGGGGAGGTGGGGGACACCCTGCTGATCATCTTCAAGAACCAGGCCAGCAGGCCCTACAACATC
TACCCCCATGGCATCACTGATGTGAGGCCCCTGTACAGCAGGAGGCTGCCCAAGGGGGTGAAGCACCT
GAAGGACTTCCCCATCCTGCCTGGGGAGATCTTCAAGTACAAGTGGACTGTGACTGTGGAGGATGGCC
CCACCAAGTCTGACCCCAGGTGCCTGACCAGATACTACAGCAGCTTTGTGAACATGGAGAGGGACCTG
GCCTCTGGCCTGATTGGCCCCCTGCTGATCTGCTACAAGGAGTCTGTGGACCAGAGGGGCAACCAGAT
CATGTCTGACAAGAGGAATGTGATCCTGTTCTCTGTGTTTGATGAGAACAGGAGCTGGTACCTGACTG
AGAACATCCAGAGGTTCCTGCCCAACCCTGCTGGGGTGCAGCTGGAGGACCCTGAGTTCCAGGCCAGC
AACATCATGCACAGCATCAATGGCTATGTGTTTGACAGCCTGCAGCTGTCTGTGTGCCTGCATGAGGT
GGCCTACTGGTACATCCTGAGCATTGGGGCCCAGACTGACTTCCTGTCTGTGTTCTTCTCTGGCTACA
CCTTCAAGCACAAGATGGTGTATGAGGACACCCTGACCCTGTTCCCCTTCTCTGGGGAGACTGTGTTC
ATGAGCATGGAGAACCCTGGCCTGTGGATTCTGGGCTGCCACAACTCTGACTTCAGGAACAGGGGCAT
GACTGCCCTGCTGAAAGTCTCCAGCTGTGACAAGAACACTGGGGACTACTATGAGGACAGCTATGAGG
ACATCTCTGCCTACCTGCTGAGCAAGAACAATGCCATTGAGCCCAGGAGCTTCAGCCAGAATAGCAGG
CACCCCAGCACCAGGCAGAAGCAGTTCAATGCCACCACCATCCCAGAGAATACCACCCTGCAGTCTGA
CCAGGAGGAGATTGACTATGATGACACCATCTCTGTGGAGATGAAGAAGGAGGACTTTGACATCTACG
ACGAGGACGAGAACCAGAGCCCCAGGAGCTTCCAGAAGAAGACCAGGCACTACTTCATTGCTGCTGTG
GAGAGGCTGTGGGACTATGGCATGAGCAGCAGCCCCCATGTGCTGAGGAACAGGGCCCAGTCTGGCTC
TGTGCCCCAGTTCAAGAAGGTGGTGTTCCAGGAGTTCACTGATGGCAGCTTCACCCAGCCCCTGTACA
GAGGGGAGCTGAATGAGCACCTGGGCCTGCTGGGCCCCTACATCAGGGCTGAGGTGGAGGACAACATC
ATGGTGACCTTCAGGAACCAGGCCAGCAGGCCCTACAGCTTCTACAGCAGCCTGATCAGCTATGAGGA
GGACCAGAGGCAGGGGGCTGAGCCCAGGAAGAACTTTGTGAAGCCCAATGAAACCAAGACCTACTTCT
GGAAGGTGCAGCACCACATGGCCCCCACCAAGGATGAGTTTGACTGCAAGGCCTGGGCCTACTTCTCT
GATGTGGACCTGGAGAAGGATGTGCACTCTGGCCTGATTGGCCCCCTGCTGGTGTGCCACACCAACAC
CCTGAACCCTGCCCATGGCAGGCAGGTGACTGTGCAGGAGTTTGCCCTGTTCTTCACCATCTTTGATG
AAACCAAGAGCTGGTACTTCACTGAGAACATGGAGAGGAACTGCAGGGCCCCCTGCAACATCCAGATG
GAGGACCCCACCTTCAAGGAGAACTACAGGTTCCATGCCATCAATGGCTACATCATGGACACCCTGCC
TGGCCTGGTGATGGCCCAGGACCAGAGGATCAGGTGGTACCTGCTGAGCATGGGCAGCAATGAGAACA
TCCACAGCATCCACTTCTCTGGCCATGTGTTCACTGTGAGGAAGAAGGAGGAGTACAAGATGGCCCTG
TACAACCTGTACCCTGGGGTGTTTGAGACTGTGGAGATGCTGCCCAGCAAGGCTGGCATCTGGAGGGT
GGAGTGCCTGATTGGGGAGCACCTGCATGCTGGCATGAGCACCCTGTTCCTGGTGTACAGCAACAAGT
GCCAGACCCCCCTGGGCATGGCCTCTGGCCACATCAGGGACTTCCAGATCACTGCCTCTGGCCAGTAT
GGCCAGTGGGCCCCCAAGCTGGCCAGGCTGCACTACTCTGGCAGCATCAATGCCTGGAGCACCAAGGA
GCCCTTCAGCTGGATCAAGGTGGACCTGCTGGCCCCCATGATCATCCATGGCATCAAGACCCAGGGGG
CCAGGCAGAAGTTCAGCAGCCTGTACATCAGCCAGTTCATCATCATGTACAGCCTGGATGGCAAGAAG
TGGCAGACCTACAGGGGCAACAGCACTGGCACCCTGATGGTGTTCTTTGGCAATGTGGACAGCTCTGG
CATCAAGCACAACATCTTCAACCCCCCCATCATTGCCAGATACATCAGGCTGCACCCCACCCACTACA
GCATCAGGAGCACCCTGAGGATGGAGCTGATGGGCTGTGACCTGAACAGCTGCAGCATGCCCCTGGGC
ATGGAGAGCAAGGCCATCTCTGATGCCCAGATCACTGCCAGCAGCTACTTCACCAACATGTTTGCCAC
CTGGAGCCCCAGCAAGGCCAGGCTGCACCTGCAGGGCAGGAGCAATGCCTGGAGGCCCCAGGTCAACA
ACCCCAAGGAGTGGCTGCAGGTGGACTTCCAGAAGACCATGAAGGTGACTGGGGTGACCACCCAGGGG
GTGAAGAGCCTGCTGACCAGCATGTATGTGAAGGAGTTCCTGATCAGCAGCAGCCAGGATGGCCACCA
GTGGACCCTGTTCTTCCAGAATGGCAAGGTGAAGGTGTTCCAGGGCAACCAGGACAGCTTCACCCCTG
TGGTGAACAGCCTGGACCCCCCCCTGCTGACCAGATACCTGAGGATTCACCCCCAGAGCTGGGTGCAC
CAGATTGCCCTGAGGATGGAGGTGCTGGGCTGTGAGGCCCAGGACCTGTACTGATTAATTAAGAGCAT
CTTACCGCCATTTATTCCCATATTTGTTCTGTTTTTCTTGATTTGGGTATACATTTAAATGTTAATAA
AACAAAATGGTGGGGCAATCATTTACATTTTTAGGGATATGTAATTACTAGTTCAGGTGTATTGCCAC
AAGACAAACATGTTAAGAAACTTTCCCGTTATTTACGCTCTGTTCCTGTTAATCAACCTCTGGATTAC
AAAATTTGTGAAAGATTGACTGATATTCTTAACTATGTTGCTCCTTTTACGCTGTGTGGATATGCTGC
TTTATAGCCTCTGTATCTAGCTATTGCTTCCCGTACGGCTTTCGTTTTCTCCTCCTTGTATAAATCCT
GGTTGCTGTCTCTTTTAGAGGAGTTGTGGCCCGTTGTCCGTCAACGTGGCGTGGTGTGCTCTGTGTTT
GCTGACGCAACCCCCACTGGCTGGGGCATTGCCACCACCTGTCAACTCCTTTCTGGGACTTTCGCTTT
CCCCCTCCCGATCGCCACGGCAGAACTCATCGCCGCCTGCCTTGCCCGCTGCTGGACAGGGGCTAGGT
TGCTGGGCACTGATAATTCCGTGGTGTTGTCTGTGCCTTCTAGTTGCCAGCCATCTGTTGTTTGCCCC
TCCCCCGTGCCTTCCTTGACCCTGGAAGGTGCCACTCCCACTGTCCTTTCCTAATAAAATGAGGAAAT
TGCATCGCATTGTCTGAGTAGGTGTCATTCTATTCTGGGGGGTGGGGTGGGGCAGGACAGCAAGGGGG
AGGATTGGGAAGACAATAGCAGGCATGCTGGGGATGCGGTGGGCTCTATGGCTCTAGAGCATGGCTAC
GTAGATAAGTAGCATGGCGGGTTAATCATTAACTACACCTGCAGGAGGAACCCCTAGTGATGGAGTTG
GCCACTCCCTCTCTGCGCGCTCGCTCGCTCACTGAGGGAGGCCGCCCGGGCAAAGCCCGGGCGTCGGG
CGACCTTTGGTCGCCCGGCCTCCCTC
077 (Lane 9; 6215 bp; hFVIII ORF 993-5384)
GAGGCCGCCCGGGCAAAGCCCGGGCGTCGGGCGACCTTTGGTCGCCCGGCCTCCCTCCCTCAGTGAGC
GAGCGAGCGCGCAGAGAGGGAGTGGCCAACTCCATCACTAGGGGTTCCTCCTGCAGGTGTAGTTAATG
ATTAACCCGCCATGCTACTTATCTACGTAGCCATGCTCTAGAGCCATAGAGCCCACCGCATCCCCAGC
ATGCCTGCTATTGTCTTCCCAATCCTCCCCCTTGCTGTCCTGCCCCACCCCACCCCCCAGAATAGAAT
GACACCTACTCAGACAATGCGATGCAATTTCCTCATTTTATTAGGAAAGGACAGTGGGAGTGGCACCT
TCCAGGGTCAAGGAAGGCACGGGGGAGGGGCAAACAACAGATGGCTGGCAACTAGAAGGCACAGACAA
CACCACGGAATTATCAGTGCCCAGCAACCTAGCCCCTGTCCAGCAGCGGGCAAGGCAGGCGGCGATGA
GTTCTGCCGTGGCGATCGGGAGGGGGAAAGCGAAAGTCCCAGAAAGGAGTTGACAGGTGGTGGCAATG
CCCCAGCCAGTGGGGGTTGCGTCAGCAAACACAGAGCACACCACGCCACGTTGACGGACAACGGGCCA
CAACTCCTCTAAAAGAGACAGCAACCAGGATTTATACAAGGAGGAGAAAACGAAAGCCGTACGGGAAG
CAATAGCTAGATACAGAGGCTATAAAGCAGCATATCCACACAGCGTAAAAGGAGCAACATAGTTAAGA
ATATCAGTCAATCTTTCACAAATTTTGTAATCCAGAGGTTGATTAACAGGAACAGAGCGTAAATAACG
GGAAAGTTTCTTAACATGTTTGTCTTGTGGCAATACACCTGAACTAGTAATTACATATCCCTAAAAAT
GTAAATGATTGCCCCACCATTTTGTTTTATTAACATTTAAATGTATACCCAAATCAAGAAAAACAGAA
CAAATATGGGAATAAATGGCGGTAAGATGCTCTTAATTAATCAGTACAGGTCCTGGGCCTCACAGCCC
AGCACCTCCATCCTCAGGGCAATCTGGTGCACCCAGCTCTGGGGGTGAATCCTCAGGTATCTGGTCAG
CAGGGGGGGGTCCAGGCTGTTCACCACAGGGGTGAAGCTGTCCTGGTTGCCCTGGAACACCTTCACCT
TGCCATTCTGGAAGAACAGGGTCCACTGGTGGCCATCCTGGCTGCTGCTGATCAGGAACTCCTTCACA
TACATGCTGGTCAGCAGGCTCTTCACCCCCTGGGTGGTCACCCCAGTCACCTTCATGGTCTTCTGGAA
GTCCACCTGCAGCCACTCCTTGGGGTTGTTGACCTGGGGCCTCCAGGCATTGCTCCTGCCCTGCAGGT
GCAGCCTGGCCTTGCTGGGGCTCCAGGTGGCAAACATGTTGGTGAAGTAGCTGCTGGCAGTGATCTGG
GCATCAGAGATGGCCTTGCTCTCCATGCCCAGGGGCATGCTGCAGCTGTTCAGGTCACAGCCCATCAG
CTCCATCCTCAGGGTGCTCCTGATGCTGTAGTGGGTGGGGTGCAGCCTGATGTATCTGGCAATGATGG
GGGGGTTGAAGATGTTGTGCTTGATGCCAGAGCTGTCCACATTGCCAAAGAACACCATCAGGGTGCCA
GTGCTGTTGCCCCTGTAGGTCTGCCACTTCTTGCCATCCAGGCTGTACATGATGATGAACTGGCTGAT
GTACAGGCTGCTGAACTTCTGCCTGGCCCCCTGGGTCTTGATGCCATGGATGATCATGGGGGCCAGCA
GGTCCACCTTGATCCAGCTGAAGGGCTCCTTGGTGCTCCAGGCATTGATGCTGCCAGAGTAGTGCAGC
CTGGCCAGCTTGGGGGCCCACTGGCCATACTGGCCAGAGGCAGTGATCTGGAAGTCCCTGATGTGGCC
AGAGGCCATGCCCAGGGGGGTCTGGCACTTGTTGCTGTACACCAGGAACAGGGTGCTCATGCCAGCAT
GCAGGTGCTCCCCAATCAGGCACTCCACCCTCCAGATGCCAGCCTTGCTGGGCAGCATCTCCACAGTC
TCAAACACCCCAGGGTACAGGTTGTACAGGGCCATCTTGTACTCCTCCTTCTTCCTCACAGTGAACAC
ATGGCCAGAGAAGTGGATGCTGTGGATGTTCTCATTGCTGCCCATGCTCAGCAGGTACCACCTGATCC
TCTGGTCCTGGGCCATCACCAGGCCAGGCAGGGTGTCCATGATGTAGCCATTGATGGCATGGAACCTG
TAGTTCTCCTTGAAGGTGGGGTCCTCCATCTGGATGTTGCAGGGGGCCCTGCAGTTCCTCTCCATGTT
CTCAGTGAAGTACCAGCTCTTGGTTTCATCAAAGATGGTGAAGAACAGGGCAAACTCCTGCACAGTCA
CCTGCCTGCCATGGGCAGGGTTCAGGGTGTTGGTGTGGCACACCAGCAGGGGGCCAATCAGGCCAGAG
TGCACATCCTTCTCCAGGTCCACATCAGAGAAGTAGGCCCAGGCCTTGCAGTCAAACTCATCCTTGGT
GGGGGCCATGTGGTGCTGCACCTTCCAGAAGTAGGTCTTGGTTTCATTGGGCTTCACAAAGTTCTTCC
TGGGCTCAGCCCCCTGCCTCTGGTCCTCCTCATAGCTGATCAGGCTGCTGTAGAAGCTGTAGGGCCTG
CTGGCCTGGTTCCTGAAGGTCACCATGATGTTGTCCTCCACCTCAGCCCTGATGTAGGGGCCCAGCAG
GCCCAGGTGCTCATTCAGCTCCCCTCTGTACAGGGGCTGGGTGAAGCTGCCATCAGTGAACTCCTGGA
ACACCACCTTCTTGAACTGGGGCACAGAGCCAGACTGGGCCCTGTTCCTCAGCACATGGGGGCTGCTG
CTCATGCCATAGTCCCACAGCCTCTCCACAGCAGCAATGAAGTAGTGCCTGGTCTTCTTCTGGAAGCT
CCTGGGGCTCTGGTTCTCGTCCTCGTCGTAGATGTCAAAGTCCTCCTTCTTCATCTCCACAGAGATGG
TGTCATCATAGTCAATCTCCTCCTGGTCAGACTGCAGGGTGGTATTCTCTGGGATGGTGGTGGCATTG
AACTGCTTCTGCCTGGTGCTGGGGTGCCTGCTATTCTGGCTGAAGCTCCTGGGCTCAATGGCATTGTT
CTTGCTCAGCAGGTAGGCAGAGATGTCCTCATAGCTGTCCTCATAGTAGTCCCCAGTGTTCTTGTCAC
AGCTGGAGACTTTCAGCAGGGCAGTCATGCCCCTGTTCCTGAAGTCAGAGTTGTGGCAGCCCAGAATC
CACAGGCCAGGGTTCTCCATGCTCATGAACACAGTCTCCCCAGAGAAGGGGAACAGGGTCAGGGTGTC
CTCATACACCATCTTGTGCTTGAAGGTGTAGCCAGAGAAGAACACAGACAGGAAGTCAGTCTGGGCCC
CAATGCTCAGGATGTACCAGTAGGCCACCTCATGCAGGCACACAGACAGCTGCAGGCTGTCAAACACA
TAGCCATTGATGCTGTGCATGATGTTGCTGGCCTGGAACTCAGGGTCCTCCAGCTGCACCCCAGCAGG
GTTGGGCAGGAACCTCTGGATGTTCTCAGTCAGGTACCAGCTCCTGTTCTCATCAAACACAGAGAACA
GGATCACATTCCTCTTGTCAGACATGATCTGGTTGCCCCTCTGGTCCACAGACTCCTTGTAGCAGATC
AGCAGGGGGCCAATCAGGCCAGAGGCCAGGTCCCTCTCCATGTTCACAAAGCTGCTGTAGTATCTGGT
CAGGCACCTGGGGTCAGACTTGGTGGGGCCATCCTCCACAGTCACAGTCCACTTGTACTTGAAGATCT
CCCCAGGCAGGATGGGGAAGTCCTTCAGGTGCTTCACCCCCTTGGGCAGCCTCCTGCTGTACAGGGGC
CTCACATCAGTGATGCCATGGGGGTAGATGTTGTAGGGCCTGCTGGCCTGGTTCTTGAAGATGATCAG
CAGGGTGTCCCCCACCTCCCCATACAGCAGGGGGCCCAGGATGCCAGACTCATGCTGGATGGCCTCCC
TGGTCTTGAAGGTTTCATCAGTGTAGGCCATGAACCTGACCTTCTTGTACTTCCTGCCAATCCTCTGG
GGGCCATTGTTCAGGTACTGGCTCTTGTAGCTCCTGTCATCAGGGGCCAGCACCAGGGGGGCATAGTC
CCAGTCCTCCTCCTCAGCAGCAATGTAGTGCACCCAGGTCTTGGGGTGCTTCTTGGCCACAGACCTGA
TCTGGATGAAGCTGGGGCTGTTGTCATCATCAAACCTCACCACATCCATCTCAGAGTCAGTCAGGTCA
TCATCATAGTCCTCAGCCTCCTCATTGTTCTTCATCCTCAGCTGGGGCTCCTCAGGGCAGCTGTCCAC
CTTCACATAGGCCTCCATGCCATCATGCTGGTGGCTGCTGATGTGGCAGAACAGCAGGAACTGGCCCA
GGTCCATCAGCAGGGTCTGGGCAGTCAGGAAGGTGATGGGGCTGATCTCCAGGCTGGCCTGCCTGTGG
TTCCTGACCAGGAAGGTGTGGCCCTCCAGGAAGATGCTGTGCACCTCAGGGGTGGTGCCCATGCCAAT
CACATGCCAGTACACAGACTTCCTGTGGCAGCCAATCAGGCCAGGCAGGCTCCTGTTCACATAGCCAT
TCACAGTGTGCATCTTGGGCCAGGCCCTGGCAGAGGCAGCATCCCTGTCCTGCATCAGGCTGTTCTTG
GTTTCAGAGTGCCAGCTCTTGCCCTCATCAAACACAGCAAACAGCAGGATGAACTTGTGCAGGGTCTG
GGTCTTCTCCTTGGCCAGGCTGCCCTCCCTGCACACCAGCAGGGCCCCAATCAGGCCAGAGTTCAGGT
CCTTCACCAGGTCCACATGGCTCAGGTAGCTGTAGGTCAGGCACAGGGGGTCAGAGGCCATGGGGCCA
TTCTCCTTCAGCACCTGCCACACATAGGTGTGGCTGCCCCCAGGGAACACCTTGTCATCCTCCTTCTC
CCTCTGGCTGGTCTGGTCATCATACTCAGCCCCCTCAGAGGCCTTCCAGTAGCTCACCCCCACAGCAT
GCAGGCTCACAGGGTGGCTGGCCATGTTCTTCAGGGTGATCACCACAGTGTCATACACCTCAGCCTGG
ATGGTGGGGCCCAGCAGGCCCATCCAGGGGGGCCTGGGCTTGGCAATGTTGAACAGGTGGTCAGTGAA
CTCCACAAACAGGGTCTTCTTGTACACCACAGAGGTGTTGAAGGGGAAGCTCTTGGGCACTCTGGGGG
GGAACCTGGCATCCACAGGCAGCTCCCCCAGGTCAGACTGCATGTAGTCCCAGCTCAGCTCCACAGCC
CCCAGGTAGTATCTCCTGGTGGCAGAGAAGCAGAACCTCAGCAGGCACAGGAAGAAGCAGGTGCTCAG
CTCAATCTGCATGGTGGCGGCGTTTAAACCAACCTGAAAAAAAGTGATTTCAGGCAGGTGCTCCAGGT
AATTAAACATTAATACCCCACCAACCAACCATCCCTTAAACCCTTACCTCTTCAGGAGCTTGTGGATC
TGTGTGACGGCTTCTCCTGGTGAAGGGGCTTTTATACCCCCTCCTTCCAACCCAGGCTGCTGATCCCT
GCCAAGCTGACTCCAAACCTGCTGATTCTGATTATTGACTTAGTCAACAAAAGGAGAATAAGTAACCT
ACACAAATATGAACCTTGCCTAGGGAGATTAGAGTATCGGAACACTCGCTCTACGAAATGTGCAGACA
GACGGGGATCCTCTAGAGCTACCTGCTGATCGCCCGGCCCCTGTTCAAACATGTCCTAATACTCTGTC
TCTGCAAGGGTCATCAGTAGTTTTCCATCTTACTCAACATCCTCCCAGTGGGTACCGTGGACTTAGCC
CCTGTTTGCTCCTCCGATAACTGGGGTGACCTTGGTTAATATTCACCAGCAGCCTCCCCCGGTGGACT
TAGCCCCTGTTTGCTCCTCCGATAACTGGGGTGACCTTGGTTAATATTCACCAGCAGCCTCCCCCGGT
GGACTTAGCCCCTGTTTGCTCCTCCGATAACTGGGGTGACCTTGGTTAATATTCACCAGCAGCCTCCC
CCGCGGCCGCGATAAGTAGCATGGCGGGTTAATCATTAACTACAAGGAACCCCTAGTGATGGAGCCTC
AGCTTGGCCACTCCCTCTCTGCGCGCTCGCTCGCTCACTGAGGCCGCCCGGGCAAAGCCCGGGCGTCG
GGCGACCTTTGGTCGCCCGGCCTCAIT
708 (Lanes 10-11; 4647 bp; hFIX)
CTGAGGCCGCCCGGGCAAAGCCCGGGCGTCGGGCGACCTTTGGTCGCCCGGCCTCANTGAGCGAGCGA
GCGCGCAGAGAGGGAGTGGCCAAGCTGAGGCTCCATCACTAGGGGTTCCTTGTAGTTAATGATTAACC
CGCCATGCTACTTATCGCGGCCGCGGGGGAGGCTGCTGGTGAATATTAACCAAGGTCACCCCAGTTAT
CGGAGGAGCAAACAGGGGCTAAGTCCACCGGGGGAGGCTGCTGGTGAATATTAACCAAGGTCACCCCA
GTTATCGGAGGAGCAAACAGGGGCTAAGTCCACCGGGGGAGGCTGCTGGTGAATATTAACCAAGGTCA
CCCCAGTTATCGGAGGAGCAAACAGGGGCTAAGTCCACGGTACCCACTGGGAGGATGTTGAGTAAGAT
GGAAAACTACTGATGACCCTTGCAGAGACAGAGTATTAGGACATGTTTGAACAGGGGCCGGGCGATCA
GCAGGTAGCTCTAGAGGATCCCCGTCTGTCTGCACATTTCGTAGAGCGAGTGTTCCGATACTCTAATC
TCCCTAGGCAAGGTTCATATTTGTGTAGGTTACTTATTCTCCTTTTGTTGACTAAGTCAATAATCAGA
ATCAGCAGGTTTGGAGTCAGCTTGGCAGGGATCAGCAGCCTGGGTTGGAAGGAGGGGGTATAAAAGCC
CCTTCACCAGGAGAAGCCGTCACACAGATCCACAAGCTCCTGAAGAGGTAAGGGTTTAAGGGATGGTT
GGTTGGTGGGGTATTAATGTTTAATTACCTGGAGCACCTGCCTGAAATCACTTTTTTTCAGGTTGGGT
TTAAACGCCGCCACCATGCAGAGGGTGAACATGATCATGGCTGAGAGCCCTGGCCTGATCACCATCTG
CCTGCTGGGCTACCTGCTGTCTGCTGAATGTACAGGTTTGTTTCCTTTTTTATAATACATTGAGTATG
CTTGCCTTTTAGATATAGAAATATCTGATTCTGTCTTCTTCACTAAATTTTGATTACATGATTTGACA
GCAATATTGAAGAGTCTAACAGCCAGCACCCAGGTTGGTAAGTACTGGTTCTTTGTTAGCTAGGTTTT
CTTCTTCTTCACTTTTAAAACTAAATAGATGGACAATGCTTATGATGCAATAAGGTTTAATAAACACT
GTTCAGTTCAGTATTTGGTCATGTAATTCCTGTTAAAAAACAGTCATCTCCTTGGTTTAAAAAAATTA
AAAGTGGGAAAACAAAGAAATAGCAGAATATAGTGAAAAAAAATAACCACAGTATTTTTGTTTGGACT
TACCACTTTGAAATCAAATTGGGAAACAAAAGCACAAACAGTGGCCTTATTTACACAAAAAGTCTGAT
TTTAAGATATGTGACAATTCAAGGTTTCAGAAGTATGTAAGGAGGTGTGTCTCTAATTTTTTAAATTA
TATATCTTCAATTTAAAGTTTTAGTTAAAACATAAAGATTAACCTTTCATTAGCAAGCTGTTAGTTAT
CACCAAAGCTTTTCATGGATTAGGAAAAAATCATTTTGTCTCTATCTCAAACATCTTGGAGTTGATAT
TTGGGGAAACACAATACTCAGTTGAGTTCCCTAGGGGAGAAAAGCAAGCTTAAGAATTGACACAAAGA
GTAGGAAGTTAGCTATTGCAACATATATCACTTTGTTTTTTCACAACTACAGTGACTTTATTTATTTC
CCAGAGGAAGGCATACAGGGAAGAAATTATCCCATTTGGACAAACAGCATGTTCTCACAGTAAGCACT
TATCACACTTACTTGTCAACTTTCTAGAATCAAATCTAGTAGCTGACAGTACCAGGATCAGGGGTGCC
AACCCTAAGCACCCCCAGAAAGCTGACTGGCCCTGTGGTTCCCACTCCAGACATGATGTCAGCTGTGA
AATCCACCTCCCTGGACCATAATTAGGCTTCTGTTCTTCAGGAGACATTTGTTCAAAGTCATTTGGGC
AACCATATTCTGAAAACAGCCCAGCCAGGGTGATGGATCACTTTGCAAAGATCCTCAATGAGCTATTT
TCAAGTGATGACAAAGTGTGAAGTTAAGGGCTCATTTGAGAACTTTCTTTTTCATCCAAAGTAAATTC
AAATATGATTAGAAATCTGACCTTTTATTACTGGAATTCTCTTGACTAAAAGTAAAATTGAATTTTAA
TTCCTAAATCTCCATGTGTATACAGTACTGTGGGAACATCACAGATTTTGGCTCCATGCCCTAAAGAG
AAATTGGCTTTCAGATTATTTGGATTAAAAACAAAGACTTTCTTAAGAGATGTAAAATTTTCATGATG
TTTTCTTTTTTGCTAAAACTAAAGAATTATTCTTTTACATTTCAGTTTTTCTTGATCATGAAAATGCC
AACAAAATTCTGAATAGACCAAAGAGGTATAACTCTGGCAAGCTTGAAGAGTTTGTACAGGGGAATCT
GGAGAGAGAGTGTATGGAAGAGAAGTGCAGCTTTGAGGAAGCCAGAGAAGTGTTTGAAAATACAGAGA
GAACAACTGAATTTTGGAAGCAGTATGTGGATGGTGATCAATGTGAGAGCAATCCCTGCTTGAATGGG
GGGAGCTGTAAAGATGATATCAACAGCTATGAATGTTGGTGTCCCTTTGGATTTGAGGGGAAAAACTG
TGAGCTTGATGTGACCTGTAATATCAAGAATGGCAGGTGTGAGCAATTTTGCAAGAATTCTGCTGATA
ACAAAGTGGTCTGTAGCTGCACTGAGGGATATAGGCTGGCTGAAAACCAGAAGAGCTGTGAACCTGCA
GTGCCTTTTCCCTGTGGGAGAGTGTCTGTGAGCCAAACCAGCAAGCTGACTAGGGCTGAAGCAGTCTT
TCCTGATGTAGATTATGTGAATAGCACTGAGGCTGAGACAATCCTTGACAATATCACTCAGAGCACAC
AGAGCTTCAATGACTTCACCAGGGTGGTAGGAGGGGAGGATGCCAAGCCTGGGCAGTTCCCCTGGCAG
GTAGTGCTCAATGGAAAAGTGGATGCCTTTTGTGGAGGTTCAATTGTAAATGAGAAGTGGATTGTGAC
TGCAGCCCACTGTGTGGAAACTGGAGTCAAGATTACTGTGGTGGCTGGAGAGCACAATATTGAGGAAA
CTGAGCACACTGAGCAGAAGAGGAATGTGATCAGGATTATCCCCCACCACAACTACAATGCTGCTATC
AACAAGTACAACCATGACATTGCCCTCCTGGAACTGGATGAACCCCTGGTCTTGAACAGCTATGTGAC
ACCCATCTGTATTGCTGATAAAGAGTACACCAACATCTTCTTGAAATTTGGGTCTGGATATGTGTCTG
GCTGGGGCAGGGTGTTCCATAAAGGCAGGTCTGCCCTGGTATTGCAGTATTTGAGGGTGCCTCTGGTG
GATAGAGCAACCTGCTTGCTGAGCACCAAGTTTACAATCTACAACAATATGTTCTGTGCAGGGTTCCA
TGAAGGTGGTAGAGACAGCTGCCAGGGAGATTCTGGGGGTCCCCATGTGACTGAGGTGGAGGGAACCA
GCTTCCTGACTGGGATTATCAGCTGGGGTGAGGAGTGTGCTATGAAGGGAAAGTATGGGATCTACACA
AAAGTATCCAGATATGTGAACTGGATTAAGGAGAAAACCAAGCTGACTTGATTAATTAAGAGCATCTT
ACCGCCATTTATTCCCATATTTGTTCTGTTTTTCTTGATTTGGGTATACATTTAAATGTTAATAAAAC
AAAATGGTGGGGCAATCATTTACATTTTTAGGGATATGTAATTACTAGTTCAGGTGTATTGCCACAAG
ACAAACATGTTAAGAAACTTTCCCGTTATTTACGCTCTGTTCCTGTTAATCAACCTCTGGATTACAAA
ATTTGTGAAAGATTGACTGATATTCTTAACTATGTTGCTCCTTTTACGCTGTGTGGATATGCTGCTTT
ATAGCCTCTGTATCTAGCTATTGCTTCCCGTACGGCTTTCGTTTTCTCCTCCTTGTATAAATCCTGGT
TGCTGTCTCTTTTAGAGGAGTTGTGGCCCGTTGTCCGTCAACGTGGCGTGGTGTGCTCTGTGTTTGCT
GACGCAACCCCCACTGGCTGGGGCATTGCCACCACCTGTCAACTCCTTTCTGGGACTTTCGCTTTCCC
CCTCCCGATCGCCACGGCAGAACTCATCGCCGCCTGCCTTGCCCGCTGCTGGACAGGGGCTAGGTTGC
TGGGCACTGATAATTCCGTGGTGTTGTCTGTGCCTTCTAGTTGCCAGCCATCTGTTGTTTGCCCCTCC
CCCGTGCCTTCCTTGACCCTGGAAGGTGCCACTCCCACTGTCCTTTCCTAATAAAATGAGGAAATTGC
ATCGCATTGTCTGAGTAGGTGTCATTCTATTCTGGGGGGTGGGGTGGGGCAGGACAGCAAGGGGGAGG
ATTGGGAAGACAATAGCAGGCATGCTGGGGATGCGGTGGGCTCTATGGCTCTAGAGCATGGCTACGTA
GATAAGTAGCATGGCGGGTTAATCATTAACTACACCTGCAGGAGGAACCCCTAGTGATGGAGTTGGCC
ACTCCCTCTCTGCGCGCTCGCTCGCTCACTGAGGGAGGCCGCCCGGGCAAAGCCCGGGCGTCGGGCGA
CCTTTGGTCGCCCGGCCTCCCTC
079 (Lane 10; 4648 bp; hFIX)
GAGGCCGCCCGGGCAAAGCCCGGGCGTCGGGCGACCTTTGGTCGCCCGGCCTCCCTCCCTCAGTGAGC
GAGCGAGCGCGCAGAGAGGGAGTGGCCAACTCCATCACTAGGGGTTCCTCCTGCAGGTGTAGTTAATG
ATTAACCCGCCATGCTACTTATCTACGTAGCCATGCTCTAGAGCCATAGAGCCCACCGCATCCCCAGC
ATGCCTGCTATTGTCTTCCCAATCCTCCCCCTTGCTGTCCTGCCCCACCCCACCCCCCAGAATAGAAT
GACACCTACTCAGACAATGCGATGCAATTTCCTCATTTTATTAGGAAAGGACAGTGGGAGTGGCACCT
TCCAGGGTCAAGGAAGGCACGGGGGAGGGGCAAACAACAGATGGCTGGCAACTAGAAGGCACAGACAA
CACCACGGAATTATCAGTGCCCAGCAACCTAGCCCCTGTCCAGCAGCGGGCAAGGCAGGCGGCGATGA
GTTCTGCCGTGGCGATCGGGAGGGGGAAAGCGAAAGTCCCAGAAAGGAGTTGACAGGTGGTGGCAATG
CCCCAGCCAGTGGGGGTTGCGTCAGCAAACACAGAGCACACCACGCCACGTTGACGGACAACGGGCCA
CAACTCCTCTAAAAGAGACAGCAACCAGGATTTATACAAGGAGGAGAAAACGAAAGCCGTACGGGAAG
CAATAGCTAGATACAGAGGCTATAAAGCAGCATATCCACACAGCGTAAAAGGAGCAACATAGTTAAGA
ATATCAGTCAATCTTTCACAAATTTTGTAATCCAGAGGTTGATTAACAGGAACAGAGCGTAAATAACG
GGAAAGTTTCTTAACATGTTTGTCTTGTGGCAATACACCTGAACTAGTAATTACATATCCCTAAAAAT
GTAAATGATTGCCCCACCATTTTGTTTTATTAACATTTAAATGTATACCCAAATCAAGAAAAACAGAA
CAAATATGGGAATAAATGGCGGTAAGATGCTCTTAATTAATCAAGTCAGCTTGGTTTTCTCCTTAATC
CAGTTCACATATCTGGATACTTTTGTGTAGATCCCATACTTTCCCTTCATAGCACACTCCTCACCCCA
GCTGATAATCCCAGTCAGGAAGCTGGTTCCCTCCACCTCAGTCACATGGGGACCCCCAGAATCTCCCT
GGCAGCTGTCTCTACCACCTTCATGGAACCCTGCACAGAACATATTGTTGTAGATTGTAAACTTGGTG
CTCAGCAAGCAGGTTGCTCTATCCACCAGAGGCACCCTCAAATACTGCAATACCAGGGCAGACCTGCC
TTTATGGAACACCCTGCCCCAGCCAGACACATATCCAGACCCAAATTTCAAGAAGATGTTGGTGTACT
CTTTATCAGCAATACAGATGGGTGTCACATAGCTGTTCAAGACCAGGGGTTCATCCAGTTCCAGGAGG
GCAATGTCATGGTTGTACTTGTTGATAGCAGCATTGTAGTTGTGGTGGGGGATAATCCTGATCACATT
CCTCTTCTGCTCAGTGTGCTCAGTTTCCTCAATATTGTGCTCTCCAGCCACCACAGTAATCTTGACTC
CAGTTTCCACACAGTGGGCTGCAGTCACAATCCACTTCTCATTTACAATTGAACCTCCACAAAAGGCA
TCCACTTTTCCATTGAGCACTACCTGCCAGGGGAACTGCCCAGGCTTGGCATCCTCCCCTCCTACCAC
CCTGGTGAAGTCATTGAAGCTCTGTGTGCTCTGAGTGATATTGTCAAGGATTGTCTCAGCCTCAGTGC
TATTCACATAATCTACATCAGGAAAGACTGCTTCAGCCCTAGTCAGCTTGCTGGTTTGGCTCACAGAC
ACTCTCCCACAGGGAAAAGGCACTGCAGGTTCACAGCTCTTCTGGTTTTCAGCCAGCCTATATCCCTC
AGTGCAGCTACAGACCACTTTGTTATCAGCAGAATTCTTGCAAAATTGCTCACACCTGCCATTCTTGA
TATTACAGGTCACATCAAGCTCACAGTTTTTCCCCTCAAATCCAAAGGGACACCAACATTCATAGCTG
TTGATATCATCTTTACAGCTCCCCCCATTCAAGCAGGGATTGCTCTCACATTGATCACCATCCACATA
CTGCTTCCAAAATTCAGTTGTTCTCTCTGTATTTTCAAACACTTCTCTGGCTTCCTCAAAGCTGCACT
TCTCTTCCATACACTCTCTCTCCAGATTCCCCTGTACAAACTCTTCAAGCTTGCCAGAGTTATACCTC
TTTGGTCTATTCAGAATTTTGTTGGCATTTTCATGATCAAGAAAAACTGAAATGTAAAAGAATAATTC
TTTAGTTTTAGCAAAAAAGAAAACATCATGAAAATTTTACATCTCTTAAGAAAGTCTTTGTTTTTAAT
CCAAATAATCTGAAAGCCAATTTCTCTTTAGGGCATGGAGCCAAAATCTGTGATGTTCCCACAGTACT
GTATACACATGGAGATTTAGGAATTAAAATTCAATTTTACTTTTAGTCAAGAGAATTCCAGTAATAAA
AGGTCAGATTTCTAATCATATTTGAATTTACTTTGGATGAAAAAGAAAGTTCTCAAATGAGCCCTTAA
CTTCACACTTTGTCATCACTTGAAAATAGCTCATTGAGGATCTTTGCAAAGTGATCCATCACCCTGGC
TGGGCTGTTTTCAGAATATGGTTGCCCAAATGACTTTGAACAAATGTCTCCTGAAGAACAGAAGCCTA
ATTATGGTCCAGGGAGGTGGATTTCACAGCTGACATCATGTCTGGAGTGGGAACCACAGGGCCAGTCA
GCTTTCTGGGGGTGCTTAGGGTTGGCACCCCTGATCCTGGTACTGTCAGCTACTAGATTTGATTCTAG
AAAGTTGACAAGTAAGTGTGATAAGTGCTTACTGTGAGAACATGCTGTTTGTCCAAATGGGATAATTT
CTTCCCTGTATGCCTTCCTCTGGGAAATAAATAAAGTCACTGTAGTTGTGAAAAAACAAAGTGATATA
TGTTGCAATAGCTAACTTCCTACTCTTTGTGTCAATTCTTAAGCTTGCTTTTCTCCCCTAGGGAACTC
AACTGAGTATTGTGTTTCCCCAAATATCAACTCCAAGATGTTTGAGATAGAGACAAAATGATTTTTTC
CTAATCCATGAAAAGCTTTGGTGATAACTAACAGCTTGCTAATGAAAGGTTAATCTTTATGTTTTAAC
TAAAACTTTAAATTGAAGATATATAATTTAAAAAATTAGAGACACACCTCCTTACATACTTCTGAAAC
CTTGAATTGTCACATATCTTAAAATCAGACTTTTTGTGTAAATAAGGCCACTGTTTGTGCTTTTGTTT
CCCAATTTGATTTCAAAGTGGTAAGTCCAAACAAAAATACTGTGGTTATTTTTTTTCACTATATTCTG
CTATTTCTTTGTTTTCCCACTTTTAATTTTTTTAAACCAAGGAGATGACTGTTTTTTAACAGGAATTA
CATGACCAAATACTGAACTGAACAGTGTTTATTAAACCTTATTGCATCATAAGCATTGTCCATCTATT
TAGTTTTAAAAGTGAAGAAGAAGAAAACCTAGCTAACAAAGAACCAGTACTTACCAACCTGGGTGCTG
GCTGTTAGACTCTTCAATATTGCTGTCAAATCATGTAATCAAAATTTAGTGAAGAAGACAGAATCAGA
TATTTCTATATCTAAAAGGCAAGCATACTCAATGTATTATAAAAAAGGAAACAAACCTGTACATTCAG
CAGACAGCAGGTAGCCCAGCAGGCAGATGGTGATCAGGCCAGGGCTCTCAGCCATGATCATGTTCACC
CTCTGCATGGTGGCGGCGTTTAAACCCAACCTGAAAAAAAGTGATTTCAGGCAGGTGCTCCAGGTAAT
TAAACATTAATACCCCACCAACCAACCATCCCTTAAACCCTTACCTCTTCAGGAGCTTGTGGATCTGT
GTGACGGCTTCTCCTGGTGAAGGGGCTTTTATACCCCCTCCTTCCAACCCAGGCTGCTGATCCCTGCC
AAGCTGACTCCAAACCTGCTGATTCTGATTATTGACTTAGTCAACAAAAGGAGAATAAGTAACCTACA
CAAATATGAACCTTGCCTAGGGAGATTAGAGTATCGGAACACTCGCTCTACGAAATGTGCAGACAGAC
GGGGATCCTCTAGAGCTACCTGCTGATCGCCCGGCCCCTGTTCAAACATGTCCTAATACTCTGTCTCT
GCAAGGGTCATCAGTAGTTTTCCATCTTACTCAACATCCTCCCAGTGGGTACCGTGGACTTAGCCCCT
GTTTGCTCCTCCGATAACTGGGGTGACCTTGGTTAATATTCACCAGCAGCCTCCCCCGGTGGACTTAG
CCCCTGTTTGCTCCTCCGATAACTGGGGTGACCTTGGTTAATATTCACCAGCAGCCTCCCCCGGTGGA
CTTAGCCCCTGTTTGCTCCTCCGATAACTGGGGTGACCTTGGTTAATATTCACCAGCAGCCTCCCCCG
CGGCCGCGATAAGTAGCATGGCGGGTTAATCATTAACTACAAGGAACCCCTAGTGATGGAGCCTCAGC
TTGGCCACTCCCTCTCTGCGCGCTCGCTCGCTCACTGAGGCCGCCCGGGCAAAGCCCGGGCGTCGGGC
GACCTTTGGTCGCCCGGCCTCAIT
709 (Lanes 12-13; 3605 bp; Luciferase)
CTGAGGCCGCCCGGGCAAAGCCCGGGCGTCGGGCGACCTTTGGTCGCCCGGCCTCANTGAGCGAGCGA
GCGCGCAGAGAGGGAGTGGCCAAGCTGAGGCTCCATCACTAGGGGTTCCTTGTAGTTAATGATTAACC
CACCATGCTACTTATGGCTAGGACTAGACTAGTAGGCTCAGAGGCACACAGGAGTTTCTGGGCTCACC
CTGCCCCCTTCCAACCCCTCAGTTCCCATCCTCCAGCAGCTGTTTGTGTGCTGCCTCTGAAGTCCACA
CTGAACAAACTTCAGCCTACTCATGTCCCTAAAATGGGCAAACATTGCAAGCAGCAAACAGCAAACAC
ACAGCCCTCCCTGCCTGCTGACCTTGGAGCTGGGGCAGAGGTCAGTGACCTCTCTGGGCCCATGCCAC
CTCCAACATCCACTTGACCCCTTGGAATTTCAGTGGAGAGGAGCAGAGGTTGTCCTGGTGTGGTTTAG
GTAGTGTGAGAGGGTCCAGGTTCAAAACCACTTGCTGGGTGGGGAGTTGTCAGTAAGTGGCTATGCCC
CAACCCTGAAGCCTGTTTCCCCATCTGTACAATGGAAATGATAAAGACCCCCATCTGATAGGGTTTTT
GTGGCAAATAAACATTTGGTTTTTTTGTTTTGTTTTGTTTTGTTTTTTGAGATGGAGGTTTGCTCTGT
GGCCCAGGCTGGAGTGCAGTGACACAATCTCATCTCACCACAACCTTCCCCTGCCTCATCCTCCCAAG
TAGCTGGGATTACAAGCATGTGCCACCACACCTGGCTAATTTTCTATTTTTAGTAGAGATGGGTTTCT
CCATGTTGGTCAGGCTCAGCCTCCCAAGTAACTGGGATTACAGGCCTGTGCCACCACACCTGGCTAAT
TTTTTCTATTTTTGACAGGGATGGGGTTTCACCATGTTGGTCAGGCTGGTCTAGAGGTACTGGATCTT
GCTACCAGTGGAACAGCCACTAAGGATTCTGCAGTGAGAGCAGAGGGCCAGCTAAGTGGTACTCTCCC
AGAGACTGTCTGACTCATGCCACCCCCTCCACCTTGGACACAGGACACTGTGGTTTCTGAGCCAGGTA
CAATGACTCCTTTTGGTAAGTGCAGTGGAAGCTGTACACTGCCCAGGCAAAGTGTCTGGGCAGCATAG
GCAGGTGACTCAGATCCCAGCCAGTGGACTTAGCCCCTGTTTGCTCCTCTGATAACTGGGGTGACCTT
GGTTAATATTCACCAGCAGCCTCCCCTGTTGCCCCTCTGGATCCACTGCTTAAATACAGACAAGGACA
GGGCCCTGTCTCTTCAGCTTCAGGCACCACCACTGACCTGGGACAGTGAATAATTACTCTAAGGTAAA
TATAAAATTTTTAAGTGTATAATGTGTTAAACTACTGATTCTAATTGTTTCTCTCTTTTAGATTCCAA
CCTTTGGAACTGAACCATGGAGGATGCCAAGAACATTAAGAAAGGCCCTGCCCCCTTCTACCCCCTGG
AGGATGGAACAGCAGGGGAGCAGCTGCACAAGGCCATGAAGAGATATGCCCTGGTCCCAGGCACCATT
GCCTTCACTGATGCCCACATTGAAGTTGATATCACCTATGCTGAATATTTTGAGATGTCTGTGAGACT
GGCAGAGGCTATGAAAAGGTATGGACTGAACACCAACCACAGAATTGTGGTGTGCAGTGAAAATTCCC
TGCAGTTCTTCATGCCTGTGCTTGGAGCTCTCTTCATTGGAGTAGCTGTTGCTCCAGCCAATGACATC
TACAATGAAAGAGAGCTCCTCAACTCCATGGGCATCTCCCAGCCCACTGTGGTGTTTGTGTCCAAAAA
GGGGCTGCAGAAAATTCTGAATGTGCAGAAGAAGCTGCCAATCATCCAGAAGATCATTATCATGGACA
GCAAGACAGATTACCAGGGTTTCCAGAGCATGTACACCTTTGTCACCAGCCACCTCCCTCCTGGCTTC
AATGAGTATGATTTTGTTCCAGAGAGCTTTGACAGAGATAAAACAATTGCACTGATTATGAACAGCTC
TGGCAGCACAGGTCTGCCCAAAGGTGTGGCCTTGCCCCACAGGACTGCCTGTGTCAGGTTCTCTCATG
CCAGGGACCCCATCTTTGGAAACCAGATCATCCCTGATACAGCCATCCTGTCTGTTGTGCCTTTCCAT
CATGGCTTTGGCATGTTCACCACCCTGGGCTACCTGATCTGTGGATTCAGAGTAGTGCTGATGTATAG
GTTTGAGGAGGAACTGTTCCTGAGGAGCCTTCAGGACTACAAGATCCAATCTGCTCTGCTGGTGCCCA
CCCTCTTTTCCTTCTTTGCCAAGAGCACCTTGATTGATAAGTATGACCTGAGCAACCTGCATGAAATT
GCTTCTGGAGGAGCCCCTCTGTCCAAGGAAGTGGGAGAGGCAGTGGCCAAAAGATTCCACCTGCCTGG
AATCAGACAGGGCTATGGCCTGACAGAGACAACTTCTGCCATTCTCATCACTCCAGAAGGAGATGACA
AGCCAGGAGCAGTGGGCAAAGTGGTGCCATTTTTTGAAGCCAAGGTGGTGGATCTGGACACAGGCAAG
ACTCTGGGAGTGAATCAGAGAGGTGAGCTGTGTGTGAGGGGCCCCATGATCATGTCAGGATATGTGAA
CAACCCTGAGGCCACCAATGCCCTCATTGACAAAGATGGCTGGCTGCACAGTGGAGACATTGCCTACT
GGGATGAAGATGAGCACTTCTTCATAGTGGACAGGCTGAAGTCCCTCATCAAATACAAAGGGTACCAA
GTGGCTCCTGCTGAGCTGGAGTCCATCCTGCTCCAGCACCCCAACATCTTTGATGCAGGAGTGGCAGG
CCTCCCAGATGATGATGCTGGAGAACTTCCAGCTGCTGTGGTTGTCCTGGAACATGGAAAGACCATGA
CTGAGAAGGAGATTGTTGACTATGTTGCCAGCCAGGTGACCACAGCTAAGAAGCTCAGAGGAGGGGTG
GTCTTTGTAGATGAGGTGCCCAAGGGCCTGACTGGAAAACTGGATGCCAGAAAAATCAGGGAGATCTT
GATCAAGGCAAAGAAAGGAGGGAAGATTGCTGTGTGATTAATTAAGCTTGCTTTCTTGCTGTCCAATT
TCTATTAAAGGTTCCTTTGTTCCCTAAGTCCAACTACTAAACTGGGGGATATTATGAAGGGCCTTGAG
CATCTGGATTCTGCCTCAGACATGATAAGATACATTGATGAGTTTGGACAAACCACAACTAGAATGCA
GTGAAAAAAATGCTTTATTTGTGAAATTTGTGATGCTATTGCTTTATTTGTAACCATTATAAGCTGCA
ATAAACAAGTTAACAACAACAATTGCATTCATTTTATGTTTCAGGTTCAGGGGGAGATGTGGGAGGTT
TTTTAAAGCAAGTAAAACCTCTACAAATGTGGTACTTAAGCCACAATCTGCCTCCCAGTAGTACATGA
CATTAGTTTATTAATAGCCTAGGAACCCCTAGTGATGGAGTTGGCCACTCCCTCTCTGCGCGCTCGCT
CGCTCACTGAGGGAGGCCGCCCGGGCAAAGCCCGGGCGTCGGGCGACCTTTGGTCGCCCGGCCTCCCT
C
081 (Lane 13; 3606 bp; Luciferase)
GAGGCCGCCCGGGCAAAGCCCGGGCGTCGGGCGACCTTTGGTCGCCCGGCCTCCCTCCCTCAGTGAGC
GAGCGAGCGCGCAGAGAGGGAGTGGCCAACTCCATCACTAGGGGTTCCTAGGCTATTAATAAACTAAT
GTCATGTACTACTGGGAGGCAGATTGTGGCTTAAGTACCACATTTGTAGAGGTTTTACTTGCTTTAAA
AAACCTCCCACATCTCCCCCTGAACCTGAAACATAAAATGAATGCAATTGTTGTTGTTAACTTGTTTA
TTGCAGCTTATAATGGTTACAAATAAAGCAATAGCATCACAAATTTCACAAATAAAGCATTTTTTTCA
CTGCATTCTAGTTGTGGTTTGTCCAAACTCATCAATGTATCTTATCATGTCTGAGGCAGAATCCAGAT
GCTCAAGGCCCTTCATAATATCCCCCAGTTTAGTAGTTGGACTTAGGGAACAAAGGAACCTTTAATAG
AAATTGGACAGCAAGAAAGCAAGCTTAATTAATCACACAGCAATCTTCCCTCCTTTCTTTGCCTTGAT
CAAGATCTCCCTGATTTTTCTGGCATCCAGTTTTCCAGTCAGGCCCTTGGGCACCTCATCTACAAAGA
CCACCCCTCCTCTGAGCTTCTTAGCTGTGGTCACCTGGCTGGCAACATAGTCAACAATCTCCTTCTCA
GTCATGGTCTTTCCATGTTCCAGGACAACCACAGCAGCTGGAAGTTCTCCAGCATCATCATCTGGGAG
GCCTGCCACTCCTGCATCAAAGATGTTGGGGTGCTGGAGCAGGATGGACTCCAGCTCAGCAGGAGCCA
CTTGGTACCCTTTGTATTTGATGAGGGACTTCAGCCTGTCCACTATGAAGAAGTGCTCATCTTCATCC
CAGTAGGCAATGTCTCCACTGTGCAGCCAGCCATCTTTGTCAATGAGGGCATTGGTGGCCTCAGGGTT
GTTCACATATCCTGACATGATCATGGGGCCCCTCACACACAGCTCACCTCTCTGATTCACTCCCAGAG
TCTTGCCTGTGTCCAGATCCACCACCTTGGCTTCAAAAAATGGCACCACTTTGCCCACTGCTCCTGGC
TTGTCATCTCCTTCTGGAGTGATGAGAATGGCAGAAGTTGTCTCTGTCAGGCCATAGCCCTGTCTGAT
TCCAGGCAGGTGGAATCTTTTGGCCACTGCCTCTCCCACTTCCTTGGACAGAGGGGCTCCTCCAGAAG
CAATTTCATGCAGGTTGCTCAGGTCATACTTATCAATCAAGGTGCTCTTGGCAAAGAAGGAAAAGAGG
GTGGGCACCAGCAGAGCAGATTGGATCTTGTAGTCCTGAAGGCTCCTCAGGAACAGTTCCTCCTCAAA
CCTATACATCAGCACTACTCTGAATCCACAGATCAGGTAGCCCAGGGTGGTGAACATGCCAAAGCCAT
GATGGAAAGGCACAACAGACAGGATGGCTGTATCAGGGATGATCTGGTTTCCAAAGATGGGGTCCCTG
GCATGAGAGAACCTGACACAGGCAGTCCTGTGGGGCAAGGCCACACCTTTGGGCAGACCTGTGCTGCC
AGAGCTGTTCATAATCAGTGCAATTGTTTTATCTCTGTCAAAGCTCTCTGGAACAAAATCATACTCAT
TGAAGCCAGGAGGGAGGTGGCTGGTGACAAAGGTGTACATGCTCTGGAAACCCTGGTAATCTGTCTTG
CTGTCCATGATAATGATCTTCTGGATGATTGGCAGCTTCTTCTGCACATTCAGAATTTTCTGCAGCCC
CTTTTTGGACACAAACACCACAGTGGGCTGGGAGATGCCCATGGAGTTGAGGAGCTCTCTTTCATTGT
AGATGTCATTGGCTGGAGCAACAGCTACTCCAATGAAGAGAGCTCCAAGCACAGGCATGAAGAACTGC
AGGGAATTTTCACTGCACACCACAATTCTGTGGTTGGTGTTCAGTCCATACCTTTTCATAGCCTCTGC
CAGTCTCACAGACATCTCAAAATATTCAGCATAGGTGATATCAACTTCAATGTGGGCATCAGTGAAGG
CAATGGTGCCTGGGACCAGGGCATATCTCTTCATGGCCTTGTGCAGCTGCTCCCCTGCTGTTCCATCC
TCCAGGGGGTAGAAGGGGGCAGGGCCTTTCTTAATGTTCTTGGCATCCTCCATGGTTCAGTTCCAAAG
GTTGGAATCTAAAAGAGAGAAACAATTAGAATCAGTAGTTTAACACATTATACACTTAAAAATTTTAT
ATTTACCTTAGAGTAATTATTCACTGTCCCAGGTCAGTGGTGGTGCCTGAAGCTGAAGAGACAGGGCC
CTGTCCTTGTCTGTATTTAAGCAGTGGATCCAGAGGGGCAACAGGGGAGGCTGCTGGTGAATATTAAC
CAAGGTCACCCCAGTTATCAGAGGAGCAAACAGGGGCTAAGTCCACTGGCTGGGATCTGAGTCACCTG
CCTATGCTGCCCAGACACTTTGCCTGGGCAGTGTACAGCTTCCACTGCACTTACCAAAAGGAGTCATT
GTACCTGGCTCAGAAACCACAGTGTCCTGTGTCCAAGGTGGAGGGGGTGGCATGAGTCAGACAGTCTC
TGGGAGAGTACCACTTAGCTGGCCCTCTGCTCTCACTGCAGAATCCTTAGTGGCTGTTCCACTGGTAG
CAAGATCCAGTACCTCTAGACCAGCCTGACCAACATGGTGAAACCCCATCCCTGTCAAAAATAGAAAA
AATTAGCCAGGTGTGGTGGCACAGGCCTGTAATCCCAGTTACTTGGGAGGCTGAGCCTGACCAACATG
GAGAAACCCATCTCTACTAAAAATAGAAAATTAGCCAGGTGTGGTGGCACATGCTTGTAATCCCAGCT
ACTTGGGAGGATGAGGCAGGGGAAGGTTGTGGTGAGATGAGATTGTGTCACTGCACTCCAGCCTGGGC
CACAGAGCAAACCTCCATCTCAAAAAACAAAACAAAACAAAACAAAAAAACCAAATGTTTATTTGCCA
CAAAAACCCTATCAGATGGGGGTCTTTATCATTTCCATTGTACAGATGGGGAAACAGGCTTCAGGGTT
GGGGCATAGCCACTTACTGACAACTCCCCACCCAGCAAGTGGTTTTGAACCTGGACCCTCTCACACTA
CCTAAACCACACCAGGACAACCTCTGCTCCTCTCCACTGAAATTCCAAGGGGTCAAGTGGATGTTGGA
GGTGGCATGGGCCCAGAGAGGTCACTGACCTCTGCCCCAGCTCCAAGGTCAGCAGGCAGGGAGGGCTG
TGTGTTTGCTGTTTGCTGCTTGCAATGTTTGCCCATTTTAGGGACATGAGTAGGCTGAAGTTTGTTCA
GTGTGGACTTCAGAGGCAGCACACAAACAGCTGCTGGAGGATGGGAACTGAGGGGTTGGAAGGGGGCA
GGGTGAGCCCAGAAACTCCTGTGTGCCTCTGAGCCTACTAGTCTAGTCCTAGCCATAAGTAGCATGGT
GGGTTAATCATTAACTACAAGGAACCCCTAGTGATGGAGCCTCAGCTTGGCCACTCCCTCTCTGCGCG
CTCGCTCGCTCACTGAGGCCGCCCGGGCAAAGCCCGGGCGTCGGGCGACCTTTGGTCGCCCGGCCTCA
IT

### Example 5. SsDNA Synthesis without Phosphorothioate (PS) Bonds to Terminate T7 Exonuclease

This Example describes the synthesis of ssDNA from a double-stranded ceDNA precursor using T7 exonuclease, but without the inclusion of PS bonds to terminate the T7.

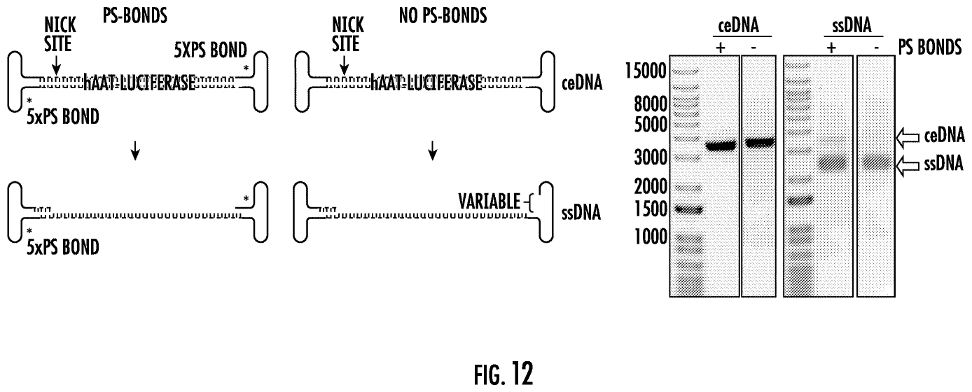

ceDNA comprising an hAAT-luciferase expression cassette, a Nb.BbvCI nick site, AAV-derived ITRs, and 5×PS bonds (FIG. 12, left) were produced as described above. Non-PS bond containing ceDNA was made similarly, except that oligonucleotides encoding the ITR structures did not include the 5× Phosphorothioate (PS) bond modifications (FIG. 12, middle). In the absence of PS bonds, T7 exonuclease was efficiently terminated, generating an ssDNA molecule of approximately the same size as ssDNA made using oligonucleotides with PS bonds (FIG. 12, right).

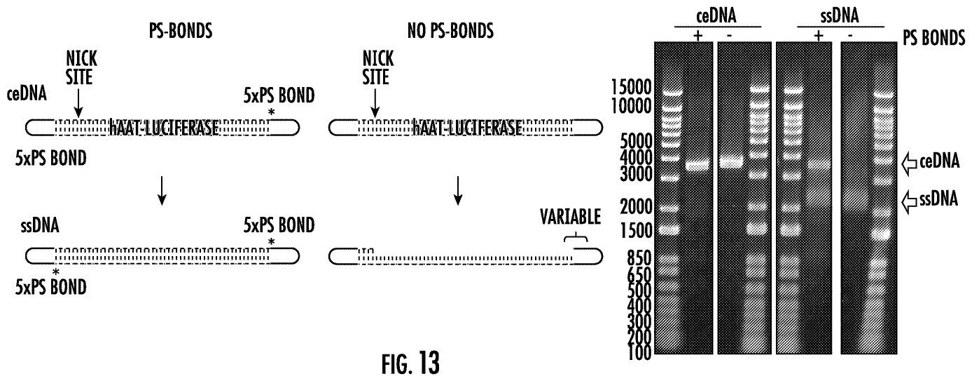

As shown in FIG. 13, termination of T7 exonuclease in the absence of PS bonds was not dependent on ITR structure. ceDNA variants were made as described above using oligonucleotides that encoded simple hairpin closed ends, with (FIG. 13, left) and without (FIG. 13, middle) PS bonds. T7 exonuclease was efficiently terminated by the hairpin structures to generate an ssDNA molecule of approximately the same size as ssDNA made using oligonucleotides with PS bonds (FIG. 13, right).

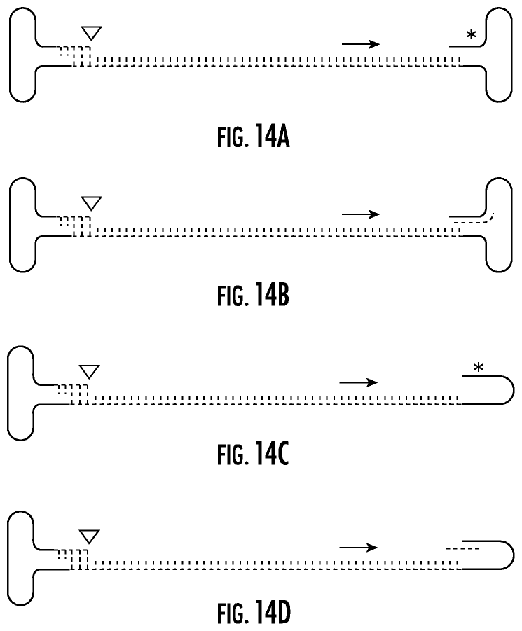

Run-off Sanger sequencing was used to map the approximate location of the termination points in PS and non-PS containing ssDNA molecules. Briefly, fully double stranded constructs were nicked (FIGS. 14A-14D, triangle), processed with T7 exo, and column purified. Sanger run-off sequencing was performed using a priming site in the single-stranded region (FIGS. 14A-14D, arrow). Sanger reads were mapped to the 5′ end of the region of interest. 5 consecutive PS bonds noted by asterisk in FIGS. 14A and 14C. Representative alignments include at least 3 Sanger reads across at least 2 independent samples. ITR ends with PS bonds (FIG. 14A) retained all PS linkages. ITR ends without PS bonds (FIG. 14B) show less protection against T7 exonuclease, with heterogeneity noted by a dotted line. Hairpin ends with PS bonds (FIG. 14C) retained all PS linkages plus one base at the 5-end. Hairpin ends without PS bonds (FIG. 14B) show less protection against T7 exonuclease, with likely heterogeneity noted by dotted line.

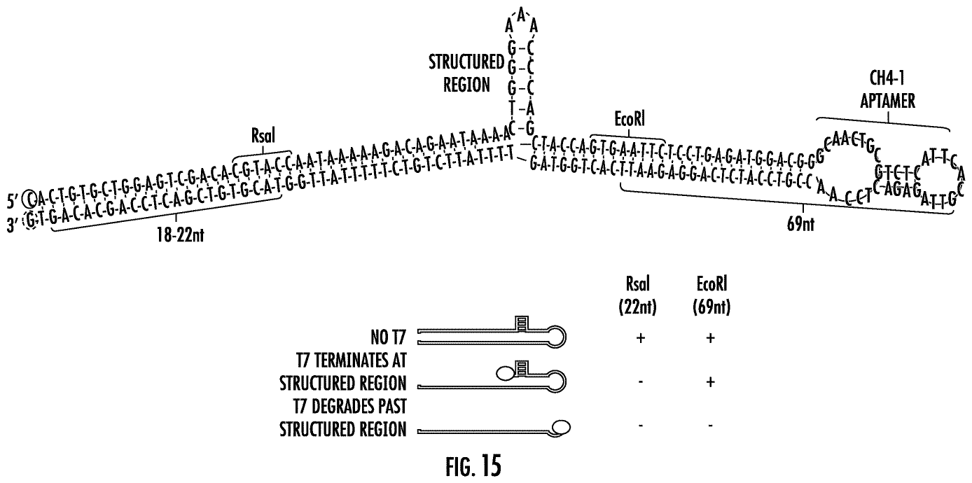

FIG. 15 illustrates an example of an end structure oligonucleotide and the strategy used to test sequence and structural requirements for T7 exonuclease termination. At the top is an example of an oligonucleotide sequence and predicted dsDNA structure. At the bottom are schematics of predicted fragments produced by RsaI and EcoRI digestion, depending on whether T7 exonuclease is terminated by the structured region.

### Example 6. Termination of T7 Exonuclease by DNA Stem and Loop Structures

A method was developed to test DNA stem and/or loop motifs for the ability to terminate T7 exonuclease. DNA oligonucleotides were designed so that, when annealed, they will form a dsDNA molecule with a stem and/or loop motif flanked by RsaI and EcoRI restriction sites. An example of such an oligonucleotide is shown in FIG. 15 (top), which comprises a stem-loop structure on the right side (in this example, the CH4-1 aptamer, which enables nuclear translocation) and a central structured region comprising a stem-loop, bubble, or loop structure flanked by an RsaI recognition site and an EcoR1 recognition site. In the absence of T7 exonuclease, RsaI and EcoRI can cleave at their dsDNA recognition sites, producing an 18-22 nt or 69 nt linear fragment, respectively (FIG. 15, bottom, “No T7”). T7 exonuclease treatment will degrade the oligonucleotide from the 5′ end, generating ssDNA. If the central stem/loop region terminates T7 exonuclease, the RsaI site is ablated and the EcoRI cleavage is maintained to generate the 69 nt linear fragment (FIG. 15, bottom “T7 terminates at structured region”. If the central stem/loop region does not terminate T7 exonuclease, neither RsaI nor EcoRI will cleave the ssDNA.

A variety of oligonucleotides were designed based on this general strategy but with different stem/loop configurations separating the RsaI and EcoRI sites (FIGS. 16A-16H). All oligonucleotides comprised the CH4-1 aptamer on the right side. The oligonucleotides were named as follows: full hilt (FIG. 16A), comprising central stem-loop structures in both strands, half hilt (FIG. 16B), comprising a central stem-loop structure in one strand, extended half hilt (FIG. 16C), comprising a longer central stem-loop structure than half hilt in one strand, bubble_v1 (FIG. 16D), comprising a small central bubble resulting in single-stranded regions in both strands, bubble_v19 (FIG. 16E), comprising a large central bubble resulting in single-stranded regions in both strands, loop (FIG. 16F), comprising a loop in one strand, PS bonds (1-5), (FIG. 16G), comprising no central structured region, but comprising 1-5 PS bonds incorporated in the central region to replace phosphodiester linkages at the different locations in the ‘5’ CTGTGA motif, and control (no TS) (FIG. 16H), comprising no central structure region.

Motifs were tested for the ability to terminate T7 Exo as follows: oligonucleotides were converted to dsDNA by denaturing at 95° C. for 5 min, followed by rapid cooling at 4° C. for 10 min. RsaI and EcoRI digests were carried on using 5 μg of annealed oligonucleotide and 10 U/μg of restriction enzyme in standard reaction conditions (NEB™, Ipswitch, MA). For T7 exonuclease pretreatment, 30 μg of annealed oligonucleotide was digested with 2.5 U/μg T7 exonuclease (NEB) in NEB Buffer 4 for 30 min at 37° C. Reactions were purified using silica spin columns before further processing with RsaI or EcoRI. Restriction reactions were quenched with 10 mM EDTA before separating on a 15% denaturing TBE-Urea PAGE gel and visualized by post staining with SYBR gold.

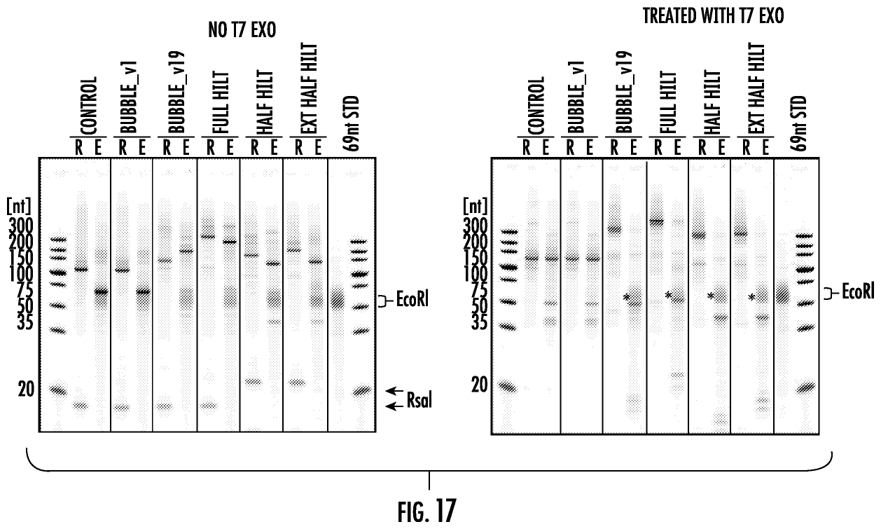

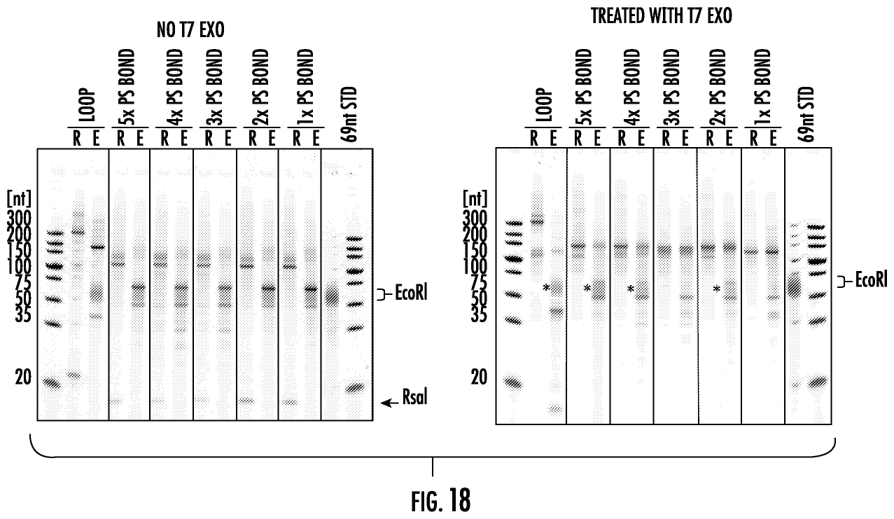

A variety of different stem, loop, and/or motifs were able to terminate T7 exonuclease including bubble_v19, half hilt, full hilt, extended half hilt (FIG. 17, compare gel on right to gel on left) and Loop (FIG. 18, compare gel on right to gel on left). PS bonds were also included as a control, showing effective termination with 4× or 5×PS bonds (FIG. 18, compare gel on right to gel on left). Not all motifs supported termination of T7 exonuclease. For instance, a 3 bp unpaired region (bubble_v1) did not support termination, whereas increasing the unpaired region to 13 bp (bubble_v19) did support termination (FIG. 17, compare gel on right to gel on left).

In general, the ability to control termination of T7 exonuclease with different stem-loop motifs enables a method of synthesizing ssDNA without the use of PS bonds while maintaining control of the specific location of the termination point. Incorporation of PS bonds into the precursor ceDNA is not always desirable or possible, so it is of value to have other methods for terminating T7 exonuclease at specific locations in the dsDNA molecule. Moreover, controlling the termination point of T7 exonuclease enables specific determination of the dsDNA stem length and the option to incorporate other structured elements such as aptamers in the dsDNA ends of the ssDNA molecule. This approach also allows the termination point for T7 exonuclease to be encoded in the dsDNA region of ceDNA that is derived from a plasmid (directly, or via RCA amplification), rather than having to terminate with PS bonds or other modifications incorporated into the oligonucleotide(s).

FIG. 19 illustrates schematic strategies for production of ssDNA using full hilt structured motifs to terminate T7 exonuclease in a double-stranded precursor with simple hairpin ends. Both sides illustrate the use of a full hilt structure. Additionally, the right side illustrates the inclusion of an aptamer encoded as double-stranded DNA, which only folds into a functional aptamer structure after the ssDNA is produced.

FIG. 20 illustrates schematic strategies for production of ssDNA using half hilt structured motifs to terminate T7 exonuclease in a double-stranded precursor with simple hairpin ends. Both sides illustrate the use of a full hilt structure. Additionally, the right side illustrates the inclusion of an aptamer encoded as double-stranded DNA, which only folds into a functional aptamer structure after the ssDNA is produced.

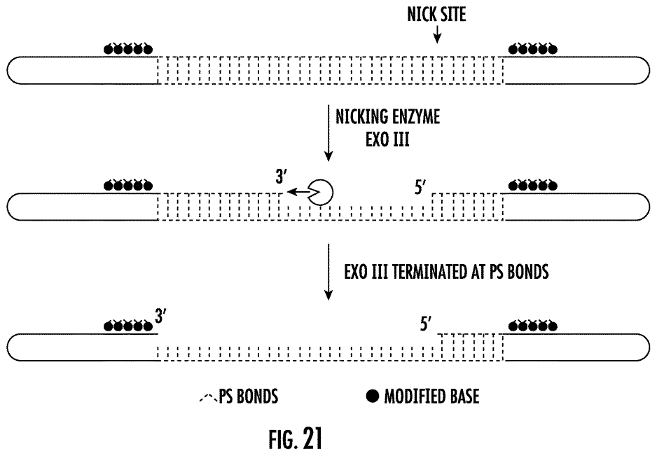

FIG. 21 illustrates a schematic strategy for production of ssDNA using Exonuclease III (Exo III) to degrade a nicked strand in the 3′→5′ direction in a double-stranded precursor with simple hairpin ends. Termination of Exo III is controlled by the specific location of PS bonds (represented by circles connected by bent lines).

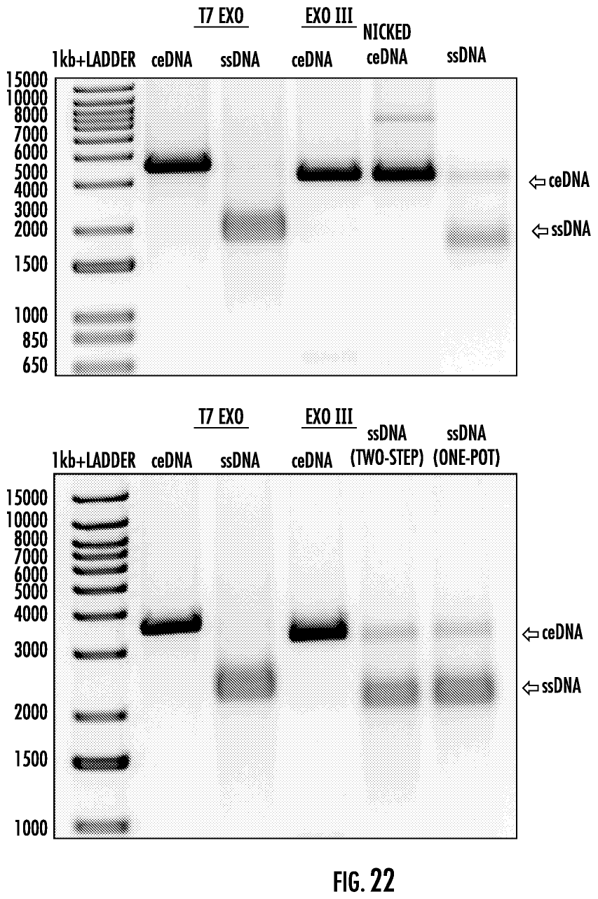

FIG. 22 shows gel analysis of ssDNA produced using Exo III, as compared to T7 exonuclease. The left shows the results of a two-step method (as depicted in FIG. 21). The right shows the results of a “one pot” method, wherein the reagents for all steps are mixed in a single reaction vessel. As shown in FIG. 22, Exo III can efficiently produce ssDNA.

### REFERENCES

All publications and references, including but not limited to patents and patent applications, cited in this specification and Examples herein are incorporated by reference in their entirety as if each individual publication or reference were specifically and individually indicated to be incorporated by reference herein as being fully set forth. Any patent application to which this application claims priority is also incorporated by reference herein in the manner described above for publications and references.

What is claimed is:

1. A method for producing a linear, single-stranded DNA (ssDNA) molecule comprising at least one nucleic acid sequence of interest flanked by at least one stem-loop structure comprising at least one stem and at least one loop at the 3′ end, the method comprising the sequential steps of:
(a) contacting a double-stranded, closed-ended DNA (ceDNA) molecule comprising the at least one nucleic acid sequence of interest with an endonuclease;
(b) contacting the double-stranded ceDNA with an exonuclease,
thereby producing the linear, ssDNA molecule.

2. The method of claim 1, wherein the ceDNA molecule further comprises at least one promoter.

3. The method of claim 2, wherein the promoter comprises a transcription start site (TSS).

4. The method of any one of claims 1-3, wherein the ceDNA molecule further comprises at least one enhancer.

5. The method of any one of claims 2-4, wherein the promoter is double-stranded in the ssDNA molecule.

6. The method of any one of claims 3-5, wherein the TSS is double-stranded in the ssDNA molecule.

7. The method of any one of claims 4-6, wherein the enhancer is double-stranded in the ssDNA molecule.

8. The method of any one of claims 1-7, wherein the ssDNA molecule further comprises at least one stem-loop structure comprising at least one stem and one loop at the 5′ end.

9. The method of any one of claims 1-8, wherein the at least one stem-loop structure at the 3′ end comprises at least two stem-loop structures, and/or wherein the at least one stem-loop structure at the 5′ end comprises at least two stem-loop structures.

10. The method of any one of claims 1-9, wherein the ceDNA molecule comprises one or more endonuclease recognition sequences.

11. The method of any one of claims 1-10, wherein the stem loop structure at the 3′ end comprises one or more endonuclease recognition sequences.

12. The method of any one of claims 8-11, wherein the stem loop structure at the 5′ end comprises one or more endonuclease recognition sequences.

13. The method of any one of claims 10-12, wherein the one or more endonuclease recognition sequences are selected from the group consisting of: 5′-CCAA-3′ (Nb.BtsI) (Nb.BsrDI) (Nt.CviPII), 5′-CCAAGC-3′ (Nb.BbvCI), 5′-CCAACC-3′ (Nb.BbvCI), 5′-CCAAGAGTCNNNN-3′ (Nt.BstNBI)-N can be A, G, C or T, 5′-CCAAG-3′ (Nb.BsmI), 5′-CCAAC-3′ (Nb.BssSI), 5′-CCAAGGATCNNNN-3′ (Nt.AlwI), CCAAGTCTCN-3′ (Nt.BsmAI), and CCAAGCTCTTCN-3′ (Nt.BspQI).

14. The method of any one of claims 1-13, wherein the terminal residue of the stem-loop structure at the 3′ end is capable of priming replication and/or transcription inside the nucleus of a host cell.

15. The method of claim 14, wherein the 3′ terminal residue comprises a free-OH.

16. The method of any one of claims 1-15, wherein contacting the double-stranded ceDNA molecule with the endonuclease creates one or more nicks in a sense strand of said nucleic acid sequence of interest, thereby creating a nicked ceDNA molecule.

17. The method of claim 16, wherein the one or more nicks in the sense strand of the nucleic acid sequence of interest are located 5′ upstream of the nucleic acid sequence of interest, within the nucleic acid sequence of interest, and/or 3′ upstream of the nucleic acid sequence of interest.

18. The method of any one of claims 16-17, wherein the one or more nicks in the sense strand of the nucleic acid sequence of interest are located 5′ upstream of the nucleic acid sequence of interest.

19. The method of any one of claims 16-18, wherein the one or more nicks in the sense strand of the nucleic acid sequence of interest are located 3′ downstream of the nucleic acid sequence of interest.

20. The method of any one of claims 16-19, wherein the one or more nicks in the sense strand of the nucleic acid sequence of interest are located within the nucleic acid sequence of interest.

21. The method of any one of claims 1-20, wherein the sense strand further comprises at least one phosphorothioate (PS) modified nucleotide downstream of said expression cassette.

22. The method any one of claims 1-21, wherein the sense strand further comprises at least 2 PS modified nucleotides downstream of said expression cassette.

23. The method any one of claims 1-22, wherein the sense strand further comprises at least 3 PS modified nucleotides downstream of said expression cassette.

24. The method any one of claims 1-23, wherein the sense strand further comprises at least 4 PS modified nucleotides downstream of said expression cassette.

25. The method any one of claims 1-24, wherein the sense strand further comprises at least 5 PS modified nucleotides downstream of said expression cassette.

26. The method of any one of claims 1-25, wherein the sense strand further comprises at least one phosphorothioate (PS) modified nucleotide upstream of said expression cassette.

27. The method any one of claims 1-26, wherein the sense strand further comprises at least 2 PS modified nucleotides upstream of said expression cassette.

28. The method any one of claims 1-27, wherein the sense strand further comprises at least 3 PS modified nucleotides upstream of said expression cassette.

29. The method any one of claims 1-28, wherein the sense strand further comprises at least 4 PS modified nucleotides upstream of said expression cassette.

30. The method any one of claims 1-29, wherein the sense strand further comprises at least 5 PS modified nucleotides upstream of said expression cassette.

31. The method of any one of claims 1-30, wherein contacting the nicked ceDNA molecule with an exonuclease creates a stretch of single-stranded DNA (ssDNA) corresponding to the nucleic acid sequence of interest in the double-stranded ceDNA molecule.

32. The method of any one of claims 1-31, wherein the endonuclease is a Type II restriction enzyme.

33. The method of any one of claims 1-32, wherein the endonuclease is selected from group consisting of Nb.BtsI, Nb.BsrDI, Nt.CviPII, Nb.BbvC1, Nt.BbvCI, Nt.BstNBI, Nb.BsmI, Nb.BssSI, Nt.AlwI, Nt.BsmA1, Nt.BspQI, and Endonuclease V (Endo V).

34. The method of any one of claims 32-33, wherein the Type II restriction enzyme is Nb.BbvCI.

35. The method of any one of claims 32-33, wherein the endonuclease is Endo V.

36. The method of any one of claims 1-35, wherein the double-stranded ceDNA molecule comprises at least one deoxyinosine residue.

37. The method of claim 36, wherein the deoxyinosine residue is present in the at least one stem-loop structure at the 3′ end, two bases upstream of a desired nick site.

38. The method of any one of claims 1-37, wherein double-stranded ceDNA molecule comprises at least one uridine-, inosine-, xanthosine-, and/or oxanosine-containing residue that is nicked by the endonuclease, wherein the endonuclease has enzymatic activity on the uridine-, inosine-, xanthosine-, and/or oxanosine-containing residue.

39. The method of any one of claims 36-38, wherein the endonuclease nicks the DNA at the second phosphodiester bond 3′ to uridine-, inosine-, xanthosine-, and/or oxanosine-containing residue.

40. The method of any one of claims 1-39, wherein the exonuclease is a T7 exonuclease.

41. The method of any one of claims 1-39, wherein the exonuclease is Exonuclease III (Exo III).

42. The method of any one of claims 1-41, further comprising the steps of:
(1) performing rolling circle amplification (RCA) using a double-stranded DNA (dsDNA) molecule, thereby producing an intermediate dsDNA molecule; and
(2) performing cell-free, enzymatic synthesis using the intermediate dsDNA molecule, thereby producing the ceDNA molecule,
wherein steps (1) and (2) are performed prior to steps (a) and (b).

43. The method of claim 42, further comprising the step of:
(3) purifying the ceDNA molecule after step (2) and prior to step (a).

44. The method of any one of claims 42-43, wherein the RCA step (1) comprises the step of:
(i) contacting the dsDNA molecule with a primer and a DNA polymerase.

45. The method of any one of claims 42-44, wherein step (2) comprises the steps of:
(i) contacting the intermediate dsDNA molecule with a restriction endonuclease to produce a cleaved intermediate dsDNA molecule,
(ii) contacting the cleaved intermediate dsDNA molecule with an oligonucleotide comprising an end compatible with at least one end the cleaved intermediate dsDNA molecule and a ligase.

46. The method of claim 45, wherein step (ii) further comprises contacting the cleaved intermediate dsDNA molecule with at least two oligonucleotides each comprising ends compatible with at least one end of the cleaved intermediate dsDNA molecule.

47. The method of claim 46, wherein the at least two oligonucleotides each comprise the same end.

48. The method of claim 46, wherein the at least two oligonucleotides each comprise different ends.

49. The method of any one of claims 46-47, wherein the at least two oligonucleotides are the same.

50. The method of any one of claim 46 or 48, wherein the at least two oligonucleotides are different.

51. The method of any one of claims 45-50, wherein step (2) further comprises the step of:
(iii) ligating the at least one oligonucleotide to the cleaved dsDNA intermediate.

52. The method of any one of claims 1-51, wherein the at least one stem at the 3′ end comprises a partial DNA duplex of between 4-500 nucleotides.

53. The method of any one of claims 1-52, wherein the at least one stem at the 3′ end comprises a partial DNA duplex of 4-5 nucleotides.

54. The method of any one of claims 8-53, wherein the at least one stem at the 5′ end comprises a partial DNA duplex of between 4-500 nucleotides.

55. The method of any one of claims 8-54, wherein the at least one stem at the 5′ end comprises a partial DNA duplex of 4-5 nucleotides.

56. The method of any one of claims 1-55, wherein the at least one loop at the 3′ end comprises between 3-500 unbound nucleotides.

57. The method of any one of claims 1-56, wherein the at least one loop at the 3′ end comprises a minimum of 3 unbound nucleotides.

58. The method of any one of claims 8-57, wherein the at least one loop at the 5′ end comprises between 3-500 unbound nucleotides.

59. The method of any one of claims 8-58, wherein the at least one loop at the 5′ end comprises a minimum of 3 unbound nucleotides.

60. The method of any one of claims 1-59, wherein the ssDNA comprises at least two stem-loop structures at the 3′ end.

61. The method of any one of claims 1-60, wherein the ssDNA comprises at least three stem-loop structures at the 3′ end.

62. The method of any one of claims 1-61, wherein the ssDNA comprises at least four or more stem-loop structures at the 3′ end.

63. The method of any one of claims 1-62, wherein the ssDNA comprises at least two stem-loop structures at the 3′ end.

64. The method of any one of claims 1-63, wherein the ssDNA comprises at least three stem-loop structures at the 3′ end.

65. The method of any one of claims 1-64, wherein the ssDNA comprises at least four or more stem-loop structures at the 3′ end.

66. The method of any one of claims 8-65, wherein the ssDNA comprises at least one bubble structure at the 5′ end.

67. The method of any one of claims 8-66, wherein the ssDNA comprises at least two stem-loop structures at the 5′ end.

68. The method of any one of claims 8-67, wherein the ssDNA comprises at least three stem-loop structures at the 5′ end.

69. The method of any one of claims 8-68, wherein the ssDNA comprises at least four or more stem-loop structures at the 5′ end.

70. The method of any one of claims 1-69, wherein the at least one stem-loop structure at the 3′ end comprises a hairpin DNA structure.

71. The method of any one of claims 1-70, wherein the at least one stem-loop structure at the 3′ end comprises a DNA structure selected from the group consisting of: a cruciform DNA structure, a hammerhead DNA structure, a quadraplex DNA structure, a bulged DNA structure, a multibranched loop structure, and a bubble structure.

72. The method of any one of claims 1-71, wherein the at least one stem-loop structure at the 3′ end does not comprise an A or A′ regions that would be present in a wild-type AAV ITR.

73. The method of any one of claims 1-72, wherein the at least one stem-loop structure at the 3′ end does not comprise an A, A′, D, or D′ region that would be present in a wild-type AAV ITR.

74. The method of any one of claims 1-73, wherein the at least one stem-loop structure at the 3′ end does not comprise an A, A′, B, B′, C, C′, D, or D′ region that would be present in a wild-type AAV ITR.

75. The method of any one of claims 8-74, wherein the at least one stem-loop structure at the 5′ end does not comprise an A or A′ region that would be present in a wild-type AAV ITR.

76. The method of any one of claims 8-75, wherein the at least one stem-loop structure at the 5′ end does not comprise an A, A′, D, or D′ region that would be present in a wild-type AAV ITR.

77. The method of any one of claims 8-76, wherein the at least one stem-loop structure at the 5′ end does not comprise an A, A′, B, B′, C, C′, D, or D′ region that would be present in a wild-type AAV ITR.

78. The method of any one of claims 1-77, wherein the at least one stem-loop structure at the 3′ end does not comprise a rep binding element (RBE) that would be present in a wild-type AAV ITR.

79. The method of any one of claims 1-78, wherein the at least one stem-loop structure at the 3′ end does not comprise a terminal resolution site (trs) that would be present in a wild-type AAV ITR.

80. The method of any one of claims 8-79, wherein the at least one stem-loop structure at the 5′ end does not comprise a rep binding element (RBE) that would be present in a wild-type AAV ITR.

81. The method of any one of claims 8-80, wherein the at least one stem-loop structure at the 5′ end does not comprise a terminal resolution site (trs) that would be present in a wild-type AAV ITR.

82. The method of any one of claims 1-81, wherein the ssDNA molecule does not comprise any virally-derived sequences.

83. The method of any one of claims 1-82, wherein the at least one stem-loop structure at the 3′ end comprises one or more nucleotides that are modified to be exonuclease resistant.

84. The method of claim 83, wherein the nucleotides that are modified to be exonuclease resistant are selected from the group consisting of phosphorothioate-modified nucleotides, locked nucleic acid (LNA)-modified nucleotides, 2′-O-methyl (m)-modified nucleotides, 2′-O-methoxy ethyl (E)-modified nucleotides, 2′-fluoro (F)-modified nucleotides, and combinations thereof.

85. The method of any one of claims 1-84, wherein the at least one stem-loop structure at the 3′ end and/or the at least one stem-loop structure at the 5′ end each independently comprise a functional moiety.

86. The method of any one of claims 8-85, wherein the at least one stem-loop structure at the 5′ end comprises a hairpin DNA structure.

87. The method of any one of claims 8-86, wherein the at least one stem-loop structure at the 5′ end comprises a DNA structure selected from the group consisting of: a cruciform DNA structure, a hammerhead DNA structure, a quadraplex DNA structure, a bulged DNA structure, a multibranched loop structure, and a bubble structure.

88. The method of any one of claims 8-87, wherein the stem structure at the 5′ end comprises one or more nucleotides that are modified to be exonuclease resistant.

89. The method of claim 88, wherein the nucleotides that are modified to be exonuclease resistant are PS modified nucleotides.

90. The method of any one of claims 8-89, wherein the at least one loop structure at the 5′ end further comprises one or more nucleic acids to stabilize the ends.

91. The method of any one of claims 8-90, wherein the at least one loop structure at the 5′ end further comprises one or more nucleic acids that are chemically modified.

92. The method of any one of claims 36-91, wherein the deoxyinosine residue is present at the position of -1i, -2i, -5i, or -7i relative to SEQ ID NO: 7.

93. The method of any one of claims 36-92, wherein the deoxyinosine residue is present at the position of -1i or -7i relative to SEQ ID NO: 7.

94. The method of any one of claims 1-93, wherein the ssDNA molecule is capable of being transported across the nuclear membrane from the cytosol into the nucleus of a host cell.

95. The method of any one of claims 1-94, wherein the ssDNA molecule further comprises at least one functional moiety.

96. The method of any one of claims 1-94, wherein the at least one stem loop structure at the 3′ end comprises at least one functional moiety.

97. The method of any one of claims 8-96, wherein the at least one stem-loop structure at the 5′ end comprises at least one functional moiety.

98. The method of any one of claims 95-97, wherein the at least one functional moiety is an aptamer.

99. The method of any one of claims 8-98, wherein the loops at the 5′ and/or 3′ ends further comprise one or more aptamers.

100. The method of any one of claims 98-99, wherein the aptamer is encoded in the ceDNA molecule, and wherein the aptamer forms a secondary aptamer structure in the ssDNA molecule.

101. The method of any one of claims 98-100, wherein the aptamer is a CH4-1 aptamer.

102. The method of any one of claims 1-101, wherein the at least one loop at the 3′ and/or 5′ ends further comprise one or more synthetic ribozymes.

103. The method of any one of claims 101-102, wherein the at least one loop at the 3′ and/or 5′ ends further comprise one or more antisense oligonucleotides (ASOs).

104. The method of any one of claims 1-103, wherein the at least one loop at the 3′ and/or 5′ ends further comprise one or more short-interfering RNAs (siRNAs).

105. The method of any one of claims 1-104, wherein the at least one loop at the 3′ and/or 5′ ends further comprise one or more antiviral nucleoside analogues (ANAs).

106. The method of any one of claims 1-105, wherein the at least one loop at the 3′ and/or 5′ ends further comprise one or more triplex forming oligonucleotides.

107. The method of any one of claims 1-106, wherein the at least one loop at the 3′ and/or 5′ ends further comprise one or more gRNAs or gDNAs.

108. The method of any one of claims 1-107, wherein the at least one loop at the 3′ and/or 5′ ends further comprise one or more molecular probes.

109. The method of any one of claims 1-108, wherein the ssDNA molecule is devoid of any viral capsid protein coding sequences.

110. The method of any one of claims 1-109, wherein the ssDNA molecule comprises a first ITR and a second ITR, and wherein the ITRs do not comprise any virally derived sequences.

111. The method of any one of claims 1-110, wherein the ssDNA molecule does not comprise any virally-derived sequences.

112. The method of any one of claims 1-111, wherein the ssDNA molecule comprises a first ITR and a second ITR, and wherein the ITRs are synthetic.

113. The method of any one of claims 1-112, wherein the ssDNA molecule is synthetically produced in vitro.

114. The method of any one of claims 1-113, wherein the ssDNA molecule is synthetically produced in vitro in a cell-free environment.

115. The method of any one of claims 1-114, wherein the ssDNA molecule does not activate or minimally activates an immune pathway.

116. The method of claim 115, wherein the immune pathway is an innate immune pathway.

117. The method of any one of claims 115-116, wherein the immune pathway is an innate immune pathway selected from the group consisting of the cGAS/STING pathway, the TLR9 pathway, an inflammasome-mediated pathway, and combinations thereof.

118. The method of any one of claims 1-117, wherein the nucleic acid sequence of interest is a therapeutic protein or a therapeutic fragment thereof.

119. The method of claim 118, wherein the at least one therapeutic protein is selected from the group consisting of an antibody, an enzyme, a coagulation factor, a transcription factor, a replication factor, a growth factor, a hormone, and a fusion protein.

120. The method of any one of claims 118-119, wherein the at least one therapeutic protein is useful for treating a genetic disorder selected from the group consisting of melanoma, hemophilia A (clotting factor VIII (FVIII) deficiency) and hemophilia B (clotting factor IX (FIX) deficiency), cystic fibrosis (CFTR), familial hypercholesterolemia (LDL receptor defect), hepatoblastoma, Wilson disease, phenylketonuria (PKU), congenital hepatic porphyria, inherited disorders of hepatic metabolism, Lesch Nyhan syndrome, sickle cell anemia, thalassaemias, xeroderma pigmentosum, Fanconi's anemia, retinitis pigmentosa, ataxia telangiectasia, Bloom's syndrome, retinoblastoma, mucopolysaccharide storage diseases (e.g., Hurler syndrome (MPS Type I), Scheie syndrome (MPS Type I S), Hurler-Scheie syndrome (MPS Type I H-S), Hunter syndrome (MPS Type II), Sanfilippo Types A, B, C, and D (MPS Types III A, B, C, and D), Morquio Types A and B (MPS IVA and MPS IVB), Maroteaux-Lamy syndrome (MPS Type VI), Sly syndrome (MPS Type VII), hyaluronidase deficiency (MPS Type IX)), Niemann-Pick Disease Types A/B, C1 and C2, Fabry disease, Schindler disease, GM2-gangliosidosis Type II (Sandhoff Disease), Tay-Sachs disease, Metachromatic Leukodystrophy, Krabbe disease, Mucolipidosis Type I, II/III and IV, Sialidosis Types I and II, Glycogen Storage disease Types I and II (Pompe disease), Gaucher disease Types I, II and III, Fabry disease, cystinosis, Batten disease, Aspartylglucosaminuria, Salla disease, Danon disease (LAMP-2 deficiency), Lysosomal Acid Lipase (LAL) deficiency, neuronal ceroid lipofuscinoses (CLN1-8, INCL, and LINCL), sphingolipidoses, galactosialidosis, amyotrophic lateral sclerosis (ALS), Parkinson's disease, Alzheimer's disease, Huntington's disease, spinocerebellar ataxia, spinal muscular atrophy, Friedreich's ataxia, Duchenne muscular dystrophy (DMD), Becker muscular dystrophies (BMD), dystrophic epidermolysis bullosa (DEB), ectonucleotide pyrophosphatase 1 deficiency, generalized arterial calcification of infancy (GACI), Leber Congenital Amaurosis, Stargardt macular dystrophy (ABCA4), ornithine transcarbamylase (OTC) deficiency, Usher syndrome, alpha-1 antitrypsin deficiency, progressive familial intrahepatic cholestasis (PFIC) type I (ATP8B1 deficiency), type II (ABCB11), type III (ABCB4), or type IV (TJP2) and Cathepsin A deficiency.

121. A linear, single-stranded DNA (ssDNA) molecule comprising at least one nucleic acid sequence of interest flanked by at least one stem-loop structure at the 3′ end produced by the method of any one of claims 1-120.

122. A lipid nanoparticle comprising the ssDNA molecule of claim 121 and a lipid.

123. A pharmaceutical composition comprising the ssDNA molecule of claim 121 or the lipid nanoparticle composition of claim 122 and a pharmaceutically acceptable excipient.

124. A host cell comprising the ssDNA molecule of claim 121 or the lipid nanoparticle of claim 122.

125. A method of treating a genetic disorder in a subject comprising administering a therapeutically effective amount of the ssDNA molecule of claim 121, the lipid nanoparticle of claim 122, or the pharmaceutical composition of claim 123 to the subject.

126. A method of delivering a therapeutic gene and/or a therapeutic protein to a subject comprising administering a therapeutically effective amount of the ssDNA molecule of claim 121, the lipid nanoparticle of claim 122, or the pharmaceutical composition of claim 123 to the subject.

127. A method of delivering a therapeutic gene and/or a therapeutic protein to a cell comprising contacting the cell with the ssDNA molecule of claim 121, the lipid nanoparticle of claim 122, or the pharmaceutical composition of claim 123, thereby delivering the therapeutic gene and/or therapeutic protein to the cell.

128. A method of delivering a therapeutic gene to the nucleus of a cell comprising contacting the cell with the ssDNA molecule of claim 121, the lipid nanoparticle of claim 122, or the pharmaceutical composition of claim 123, thereby delivering the therapeutic gene and/or therapeutic protein to the nucleus of the cell.

129. A method of minimizing an immune response in a subject, wherein the subject is being treated with a therapeutic gene or therapeutic protein, comprising administering a therapeutically effective amount of the ssDNA molecule of claim 121, the lipid nanoparticle of claim 122, or the pharmaceutical composition of claim 123 to the subject, wherein the nucleic acid of interest encodes the therapeutic gene or therapeutic protein.


SEQUENCE LISTING (69 sequences)

SEQ ID NO: 1 - DNA, 6242 bp, synthetic construct
  source 1..6242: mol_type = other DNA
gaggccgcccgggcaaagcccgggcgtcgggcgacctttggtcgcccggcctcagtgagcgagcgagcgcgcagagagggagtggccaactccatcactaggggttcctcctgcaggtgtagttaatgattaacccgccatgctacttatctacgtagccatgctctagagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagacaacaccacggaattatcagtgcccagcaacctagcccctgtccagcagcgggcaaggcaggcggcgatgagttctgccgtggcgatcgggagggggaaagcgaaagtcccagaaaggagttgacaggtggtggcaatgccccagccagtgggggttgcgtcagcaaacacagagcacaccacgccacgttgacggacaacgggccacaactcctctaaaagagacagcaaccaggatttatacaaggaggagaaaacgaaagccgtacgggaagcaatagctagatacagaggctataaagcagcatatccacacagcgtaaaaggagcaacatagttaagaatatcagtcaatctttcacaaattttgtaatccagaggttgattaacaggaacagagcgtaaataacgggaaagtttcttaacatgtttgtcttgtggcaatacacctgaactagtaattacatatccctaaaaatgtaaatgattgccccaccattttgttttattaacatttaaatgtatacccaaatcaagaaaaacagaacaaatatgggaataaatggcggtaagatgctcttaattaatcagtacaggtcctgggcctcacagcccagcacctccatcctcagggcaatctggtgcacccagctctgggggtgaatcctcaggtatctggtcagcaggggggggtccaggctgttcaccacaggggtgaagctgtcctggttgccctggaacaccttcaccttgccattctggaagaacagggtccactggtggccatcctggctgctgctgatcaggaactccttcacatacatgctggtcagcaggctcttcaccccctgggtggtcaccccagtcaccttcatggtcttctggaagtccacctgcagccactccttggggttgttgacctggggcctccaggcattgctcctgccctgcaggtgcagcctggccttgctggggctccaggtggcaaacatgttggtgaagtagctgctggcagtgatctgggcatcagagatggccttgctctccatgcccaggggcatgctgcagctgttcaggtcacagcccatcagctccatcctcagggtgctcctgatgctgtagtgggtggggtgcagcctgatgtatctggcaatgatgggggggttgaagatgttgtgcttgatgccagagctgtccacattgccaaagaacaccatcagggtgccagtgctgttgcccctgtaggtctgccacttcttgccatccaggctgtacatgatgatgaactggctgatgtacaggctgctgaacttctgcctggccccctgggtcttgatgccatggatgatcatgggggccagcaggtccaccttgatccagctgaagggctccttggtgctccaggcattgatgctgccagagtagtgcagcctggccagcttgggggcccactggccatactggccagaggcagtgatctggaagtccctgatgtggccagaggccatgcccaggggggtctggcacttgttgctgtacaccaggaacagggtgctcatgccagcatgcaggtgctccccaatcaggcactccaccctccagatgccagccttgctgggcagcatctccacagtctcaaacaccccagggtacaggttgtacagggccatcttgtactcctccttcttcctcacagtgaacacatggccagagaagtggatgctgtggatgttctcattgctgcccatgctcagcaggtaccacctgatcctctggtcctgggccatcaccaggccaggcagggtgtccatgatgtagccattgatggcatggaacctgtagttctccttgaaggtggggtcctccatctggatgttgcagggggccctgcagttcctctccatgttctcagtgaagtaccagctcttggtttcatcaaagatggtgaagaacagggcaaactcctgcacagtcacctgcctgccatgggcagggttcagggtgttggtgtggcacaccagcagggggccaatcaggccagagtgcacatccttctccaggtccacatcagagaagtaggcccaggccttgcagtcaaactcatccttggtgggggccatgtggtgctgcaccttccagaagtaggtcttggtttcattgggcttcacaaagttcttcctgggctcagccccctgcctctggtcctcctcatagctgatcaggctgctgtagaagctgtagggcctgctggcctggttcctgaaggtcaccatgatgttgtcctccacctcagccctgatgtaggggcccagcaggcccaggtgctcattcagctcccctctgtacaggggctgggtgaagctgccatcagtgaactcctggaacaccaccttcttgaactggggcacagagccagactgggccctgttcctcagcacatgggggctgctgctcatgccatagtcccacagcctctccacagcagcaatgaagtagtgcctggtcttcttctggaagctcctggggctctggttctcgtcctcgtcgtagatgtcaaagtcctccttcttcatctccacagagatggtgtcatcatagtcaatctcctcctggtcagactgcagggtggtattctctgggatggtggtggcattgaactgcttctgcctggtgctggggtgcctgctattctggctgaagctcctgggctcaatggcattgttcttgctcagcaggtaggcagagatgtcctcatagctgtcctcatagtagtccccagtgttcttgtcacagctggagactttcagcagggcagtcatgcccctgttcctgaagtcagagttgtggcagcccagaatccacaggccagggttctccatgctcatgaacacagtctccccagagaaggggaacagggtcagggtgtcctcatacaccatcttgtgcttgaaggtgtagccagagaagaacacagacaggaagtcagtctgggccccaatgctcaggatgtaccagtaggccacctcatgcaggcacacagacagctgcaggctgtcaaacacatagccattgatgctgtgcatgatgttgctggcctggaactcagggtcctccagctgcaccccagcagggttgggcaggaacctctggatgttctcagtcaggtaccagctcctgttctcatcaaacacagagaacaggatcacattcctcttgtcagacatgatctggttgcccctctggtccacagactccttgtagcagatcagcagggggccaatcaggccagaggccaggtccctctccatgttcacaaagctgctgtagtatctggtcaggcacctggggtcagacttggtggggccatcctccacagtcacagtccacttgtacttgaagatctccccaggcaggatggggaagtccttcaggtgcttcacccccttgggcagcctcctgctgtacaggggcctcacatcagtgatgccatgggggtagatgttgtagggcctgctggcctggttcttgaagatgatcagcagggtgtcccccacctccccatacagcagggggcccaggatgccagactcatgctggatggcctccctggtcttgaaggtttcatcagtgtaggccatgaacctgaccttcttgtacttcctgccaatcctctgggggccattgttcaggtactggctcttgtagctcctgtcatcaggggccagcaccaggggggcatagtcccagtcctcctcctcagcagcaatgtagtgcacccaggtcttggggtgcttcttggccacagacctgatctggatgaagctggggctgttgtcatcatcaaacctcaccacatccatctcagagtcagtcaggtcatcatcatagtcctcagcctcctcattgttcttcatcctcagctggggctcctcagggcagctgtccaccttcacataggcctccatgccatcatgctggtggctgctgatgtggcagaacagcaggaactggcccaggtccatcagcagggtctgggcagtcaggaaggtgatggggctgatctccaggctggcctgcctgtggttcctgaccaggaaggtgtggccctccaggaagatgctgtgcacctcaggggtggtgcccatgccaatcacatgccagtacacagacttcctgtggcagccaatcaggccaggcaggctcctgttcacatagccattcacagtgtgcatcttgggccaggccctggcagaggcagcatccctgtcctgcatcaggctgttcttggtttcagagtgccagctcttgccctcatcaaacacagcaaacagcaggatgaacttgtgcagggtctgggtcttctccttggccaggctgccctccctgcacaccagcagggccccaatcaggccagagttcaggtccttcaccaggtccacatggctcaggtagctgtaggtcaggcacagggggtcagaggccatggggccattctccttcagcacctgccacacataggtgtggctgcccccagggaacaccttgtcatcctccttctccctctggctggtctggtcatcatactcagccccctcagaggccttccagtagctcacccccacagcatgcaggctcacagggtggctggccatgttcttcagggtgatcaccacagtgtcatacacctcagcctggatggtggggcccagcaggcccatccaggggggcctgggcttggcaatgttgaacaggtggtcagtgaactccacaaacagggtcttcttgtacaccacagaggtgttgaaggggaagctcttgggcactctgggggggaacctggcatccacaggcagctcccccaggtcagactgcatgtagtcccagctcagctccacagcccccaggtagtatctcctggtggcagagaagcagaacctcagcaggcacaggaagaagcaggtgctcagctcaatctgcatggtggcggcgtttaaaccaacctgaaaaaaagtgatttcaggcaggtgctccaggtaattaaacattaataccccaccaaccaaccatcccttaaacccttacctcttcaggagcttgtggatctgtgtgacggcttctcctggtgaaggggcttttataccccctccttccaacccaggctgctgatccctgccaagctgactccaaacctgctgattctgattattgacttagtcaacaaaaggagaataagtaacctacacaaatatgaaccttgcctagggagattagagtatcggaacactcgctctacgaaatgtgcagacagacggggatcctctagagctacctgctgatcgcccggcccctgttcaaacatgtcctaatactctgtctctgcaagggtcatcagtagttttccatcttactcaacatcctcccagtgggtaccgtggacttagcccctgtttgctcctccgataactggggtgaccttggttaatattcaccagcagcctcccccggtggacttagcccctgtttgctcctccgataactggggtgaccttggttaatattcaccagcagcctcccccggtggacttagcccctgtttgctcctccgataactggggtgaccttggttaatattcaccagcagcctcccccgcggccgcgataagtagcatggcgggttaatcattaactacaaggaacccctagtgatggagcctcagcttggccactccctctctgcgcgctcgctcgctcactgaggccgcccgggcaaagcccgggcgtcgggcgacctttggtcgcccggcctcagtgagcgagcgagcgcgcagagagggagtggccaagc

SEQ ID NO: 2 - DNA, 6242 bp, synthetic construct; identical to SEQ ID NO: 1
  source 1..6242: mol_type = other DNA

SEQ ID NO: 3 - DNA, 6201 bp, synthetic construct
  source 1..6201: mol_type = other DNA
  modified_base 6200: mod_base = OTHER; note = Inosine
gaggccgcccgggcaaagcccgggcgtcgggcgacctttggtcgcccggcctcagtgagcgagcgagcgcgcagagagggagtggccaactccatcactaggggttcctcctgcaggtgtagttaatgattaacccgccatgctacttatctacgtagccatgctctagagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagacaacaccacggaattatcagtgcccagcaacctagcccctgtccagcagcgggcaaggcaggcggcgatgagttctgccgtggcgatcgggagggggaaagcgaaagtcccagaaaggagttgacaggtggtggcaatgccccagccagtgggggttgcgtcagcaaacacagagcacaccacgccacgttgacggacaacgggccacaactcctctaaaagagacagcaaccaggatttatacaaggaggagaaaacgaaagccgtacgggaagcaatagctagatacagaggctataaagcagcatatccacacagcgtaaaaggagcaacatagttaagaatatcagtcaatctttcacaaattttgtaatccagaggttgattaacaggaacagagcgtaaataacgggaaagtttcttaacatgtttgtcttgtggcaatacacctgaactagtaattacatatccctaaaaatgtaaatgattgccccaccattttgttttattaacatttaaatgtatacccaaatcaagaaaaacagaacaaatatgggaataaatggcggtaagatgctcttaattaatcagtacaggtcctgggcctcacagcccagcacctccatcctcagggcaatctggtgcacccagctctgggggtgaatcctcaggtatctggtcagcaggggggggtccaggctgttcaccacaggggtgaagctgtcctggttgccctggaacaccttcaccttgccattctggaagaacagggtccactggtggccatcctggctgctgctgatcaggaactccttcacatacatgctggtcagcaggctcttcaccccctgggtggtcaccccagtcaccttcatggtcttctggaagtccacctgcagccactccttggggttgttgacctggggcctccaggcattgctcctgccctgcaggtgcagcctggccttgctggggctccaggtggcaaacatgttggtgaagtagctgctggcagtgatctgggcatcagagatggccttgctctccatgcccaggggcatgctgcagctgttcaggtcacagcccatcagctccatcctcagggtgctcctgatgctgtagtgggtggggtgcagcctgatgtatctggcaatgatgggggggttgaagatgttgtgcttgatgccagagctgtccacattgccaaagaacaccatcagggtgccagtgctgttgcccctgtaggtctgccacttcttgccatccaggctgtacatgatgatgaactggctgatgtacaggctgctgaacttctgcctggccccctgggtcttgatgccatggatgatcatgggggccagcaggtccaccttgatccagctgaagggctccttggtgctccaggcattgatgctgccagagtagtgcagcctggccagcttgggggcccactggccatactggccagaggcagtgatctggaagtccctgatgtggccagaggccatgcccaggggggtctggcacttgttgctgtacaccaggaacagggtgctcatgccagcatgcaggtgctccccaatcaggcactccaccctccagatgccagccttgctgggcagcatctccacagtctcaaacaccccagggtacaggttgtacagggccatcttgtactcctccttcttcctcacagtgaacacatggccagagaagtggatgctgtggatgttctcattgctgcccatgctcagcaggtaccacctgatcctctggtcctgggccatcaccaggccaggcagggtgtccatgatgtagccattgatggcatggaacctgtagttctccttgaaggtggggtcctccatctggatgttgcagggggccctgcagttcctctccatgttctcagtgaagtaccagctcttggtttcatcaaagatggtgaagaacagggcaaactcctgcacagtcacctgcctgccatgggcagggttcagggtgttggtgtggcacaccagcagggggccaatcaggccagagtgcacatccttctccaggtccacatcagagaagtaggcccaggccttgcagtcaaactcatccttggtgggggccatgtggtgctgcaccttccagaagtaggtcttggtttcattgggcttcacaaagttcttcctgggctcagccccctgcctctggtcctcctcatagctgatcaggctgctgtagaagctgtagggcctgctggcctggttcctgaaggtcaccatgatgttgtcctccacctcagccctgatgtaggggcccagcaggcccaggtgctcattcagctcccctctgtacaggggctgggtgaagctgccatcagtgaactcctggaacaccaccttcttgaactggggcacagagccagactgggccctgttcctcagcacatgggggctgctgctcatgccatagtcccacagcctctccacagcagcaatgaagtagtgcctggtcttcttctggaagctcctggggctctggttctcgtcctcgtcgtagatgtcaaagtcctccttcttcatctccacagagatggtgtcatcatagtcaatctcctcctggtcagactgcagggtggtattctctgggatggtggtggcattgaactgcttctgcctggtgctggggtgcctgctattctggctgaagctcctgggctcaatggcattgttcttgctcagcaggtaggcagagatgtcctcatagctgtcctcatagtagtccccagtgttcttgtcacagctggagactttcagcagggcagtcatgcccctgttcctgaagtcagagttgtggcagcccagaatccacaggccagggttctccatgctcatgaacacagtctccccagagaaggggaacagggtcagggtgtcctcatacaccatcttgtgcttgaaggtgtagccagagaagaacacagacaggaagtcagtctgggccccaatgctcaggatgtaccagtaggccacctcatgcaggcacacagacagctgcaggctgtcaaacacatagccattgatgctgtgcatgatgttgctggcctggaactcagggtcctccagctgcaccccagcagggttgggcaggaacctctggatgttctcagtcaggtaccagctcctgttctcatcaaacacagagaacaggatcacattcctcttgtcagacatgatctggttgcccctctggtccacagactccttgtagcagatcagcagggggccaatcaggccagaggccaggtccctctccatgttcacaaagctgctgtagtatctggtcaggcacctggggtcagacttggtggggccatcctccacagtcacagtccacttgtacttgaagatctccccaggcaggatggggaagtccttcaggtgcttcacccccttgggcagcctcctgctgtacaggggcctcacatcagtgatgccatgggggtagatgttgtagggcctgctggcctggttcttgaagatgatcagcagggtgtcccccacctccccatacagcagggggcccaggatgccagactcatgctggatggcctccctggtcttgaaggtttcatcagtgtaggccatgaacctgaccttcttgtacttcctgccaatcctctgggggccattgttcaggtactggctcttgtagctcctgtcatcaggggccagcaccaggggggcatagtcccagtcctcctcctcagcagcaatgtagtgcacccaggtcttggggtgcttcttggccacagacctgatctggatgaagctggggctgttgtcatcatcaaacctcaccacatccatctcagagtcagtcaggtcatcatcatagtcctcagcctcctcattgttcttcatcctcagctggggctcctcagggcagctgtccaccttcacataggcctccatgccatcatgctggtggctgctgatgtggcagaacagcaggaactggcccaggtccatcagcagggtctgggcagtcaggaaggtgatggggctgatctccaggctggcctgcctgtggttcctgaccaggaaggtgtggccctccaggaagatgctgtgcacctcaggggtggtgcccatgccaatcacatgccagtacacagacttcctgtggcagccaatcaggccaggcaggctcctgttcacatagccattcacagtgtgcatcttgggccaggccctggcagaggcagcatccctgtcctgcatcaggctgttcttggtttcagagtgccagctcttgccctcatcaaacacagcaaacagcaggatgaacttgtgcagggtctgggtcttctccttggccaggctgccctccctgcacaccagcagggccccaatcaggccagagttcaggtccttcaccaggtccacatggctcaggtagctgtaggtcaggcacagggggtcagaggccatggggccattctccttcagcacctgccacacataggtgtggctgcccccagggaacaccttgtcatcctccttctccctctggctggtctggtcatcatactcagccccctcagaggccttccagtagctcacccccacagcatgcaggctcacagggtggctggccatgttcttcagggtgatcaccacagtgtcatacacctcagcctggatggtggggcccagcaggcccatccaggggggcctgggcttggcaatgttgaacaggtggtcagtgaactccacaaacagggtcttcttgtacaccacagaggtgttgaaggggaagctcttgggcactctgggggggaacctggcatccacaggcagctcccccaggtcagactgcatgtagtcccagctcagctccacagcccccaggtagtatctcctggtggcagagaagcagaacctcagcaggcacaggaagaagcaggtgctcagctcaatctgcatggtggcggcgtttaaaccaacctgaaaaaaagtgatttcaggcaggtgctccaggtaattaaacattaataccccaccaaccaaccatcccttaaacccttacctcttcaggagcttgtggatctgtgtgacggcttctcctggtgaaggggcttttataccccctccttccaacccaggctgctgatccctgccaagctgactccaaacctgctgattctgattattgacttagtcaacaaaaggagaataagtaacctacacaaatatgaaccttgcctagggagattagagtatcggaacactcgctctacgaaatgtgcagacagacggggatcctctagagctacctgctgatcgcccggcccctgttcaaacatgtcctaatactctgtctctgcaagggtcatcagtagttttccatcttactcaacatcctcccagtgggtaccgtggacttagcccctgtttgctcctccgataactggggtgaccttggttaatattcaccagcagcctcccccggtggacttagcccctgtttgctcctccgataactggggtgaccttggttaatattcaccagcagcctcccccggtggacttagcccctgtttgctcctccgataactggggtgaccttggttaatattcaccagcagcctcccccgcggccgcgataagtagcatggcgggttaatcattaactacaaggaacccctagtgatggagcctcagcttggccactccctctctgcgcgctcgctcgctcactgaggccgcccgggcaaagcccgggcgtcgggcgacctttggtcgcccgnc

SEQ ID NO: 4 - DNA, 61 bp, synthetic construct
  source 1..61: mol_type = other DNA
ctcactgaggccgcccgggcaaagcccgggcgtcgggcgacctttggtcgcccggcctcag

SEQ ID NO: 5 - DNA, 61 bp, synthetic construct
  source 1..61: mol_type = other DNA
  modified_base 55: mod_base = OTHER; note = Inosine
ctcactgaggccgcccgggcaaagcccgggcgtcgggcgacctttggtcgcccgncctcag

SEQ ID NO: 6 - DNA, 57 bp, synthetic construct
  source 1..57: mol_type = other DNA
cactgaggccgcccgggcaaagcccgggcgtcgggcgacctttggtcgcccggcctc

SEQ ID NO: 7 - DNA, 61 bp, synthetic construct; identical to SEQ ID NO: 4
  source 1..61: mol_type = other DNA

SEQ ID NO: 8 - DNA, 6214 bp, synthetic construct
  source 1..6214: mol_type = other DNA
ctcactgaggccgcccgggcaaagcccgggcgtcgggcgacctttggtcgcccggcctcagtgagcgagcgagcgcgcagagagggagtggccaagctgaggctccatcactaggggttccttgtagttaatgattaacccgccatgctacttatcgcggccgcgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccacggtacccactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgaagaggtaagggtttaagggatggttggttggtggggtattaatgtttaattacctggagcacctgcctgaaatcactttttttcaggttggtttaaacgccgccaccatgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaatagcaggcaccccagcaccaggcagaagcagttcaatgccaccaccatcccagagaataccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactgattaattaagagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctctagagcatggctacgtagataagtagcatggcgggttaatcattaactacacctgcaggaggaacccctagtgatggagttggccactccctctctgcgcgctcgctcgctcactgaggccgggcgaccaaaggtcgcccgacgcccgggctttgcccgggcggcctcagtg

SEQ ID NO: 9 - DNA, 6214 bp, synthetic construct
  source 1..6214: mol_type = other DNA
  misc_difference 55: note = a, c, t, g, unknown or other
ctcactgaggccgcccgggcaaagcccgggcgtcgggcgacctttggtcgcccgncctcagtgagcgagcgagcgcgcagagagggagtggccaagctgaggctccatcactaggggttccttgtagttaatgattaacccgccatgctacttatcgcggccgcgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccacggtacccactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgaagaggtaagggtttaagggatggttggttggtggggtattaatgtttaattacctggagcacctgcctgaaatcactttttttcaggttggtttaaacgccgccaccatgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaatagcaggcaccccagcaccaggcagaagcagttcaatgccaccaccatcccagagaataccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactgattaattaagagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctctagagcatggctacgtagataagtagcatggcgggttaatcattaactacacctgcaggaggaacccctagtgatggagttggccactccctctctgcgcgctcgctcgctcactgaggccgggcgaccaaaggtcgcccgacgcccgggctttgcccgggcggcctcagtg

SEQ ID NO: 10 - DNA, 6040 bp, synthetic construct
  source 1..6040: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagacgggggaggctgctggtgaatattaaccaaggtcaccccagttatcggaggagcaaacaggggctaagtccacacgcgtggtaccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgaagaggtaagggtttaagggatggttggttggtggggtattaatgtttaattacctggagcacctgcctgaaatcactttttttcaggttggtttaaacgccgccaccatgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaatgccactaatgtgtctaacaacagcaacaccagcaatgacagcaatgtgtctcccccagtgctgaagaggcaccagagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactgattaattaagagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctctagagcatggctacgtagataagtagcatggcgggttaatcattaactacacctgcaggaggaacccctagtgatggagttggccactccctctctgcgcgctcgctcgctcactgaggccgggcgaccaaaggtcgcccgacgcccgggcggcctcagtgagcgagcgagcgcgcagctgcctgcagg

SEQ ID NO: 11 - DNA, 8199 bp, synthetic construct
  source 1..8199: mol_type = other DNA
aggggttccgcgcacatttccccgaaaagtgccacctgacgtctttcgtcagatcaccgggttgttccatatcatcgtgtccacaagggcttgccggtcaagtgcctaagctcactcgagggtctcgtgagcgagcgagcgcgcagagagggagtggccaagctgaggctccatcactaggggttccttgtagttaatgattaacccgccatgctacttatcgcggccgcgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccacggtacccactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgaagaggtaagggtttaagggatggttggttggtggggtattaatgtttaattacctggagcacctgcctgaaatcactttttttcaggttggtttaaacgccgccaccatgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaatagcaggcaccccagcaccaggcagaagcagttcaatgccaccaccatcccagagaataccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactgattaattaagagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctctagagcatggctacgtagataagtagcatggcgggttaatcattaactacacctgcaggaggaacccctagtgatggagttggccactccctctctgcgcgctcgctcgctcactcgagaccaccggtttacaggatagtctactcccagtgtccacatggttaggaccgaggggctttgggtttagacactggcgacttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaat

SEQ ID NO: 12 - DNA, 61 bp, synthetic construct; identical to SEQ ID NO: 4
  source 1..61: mol_type = other DNA

SEQ ID NO: 13 - DNA, 57 bp, synthetic construct; identical to SEQ ID NO: 6
  source 1..57: mol_type = other DNA

SEQ ID NO: 14 - DNA, 202 bp, synthetic construct
  source 1..202: mol_type = other DNA
ataccagcttattcaattggtctcgcacttacctctacacactcggcgccaataaaaagacagaataaaagactgagatggacgggcaactgcgtctcattcacgttagagactccaaccgtccatctcagaattttattctgtctttttattggcgccgagtgtgtagaggtaagtgcgagaccagatagtaagtgcaatc

SEQ ID NO: 15 - DNA, 12 bp, synthetic construct
  source 1..12: mol_type = other DNA
  misc_difference 12: note = a, c, t, g, unknown or other
ccaagctcttcn

SEQ ID NO: 16 - DNA, 16 bp, synthetic construct
  source 1..16: mol_type = other DNA
gcgcgctcgctcgctc

SEQ ID NO: 17 - protein, 7 aa, Simian virus 40
PKKKRKV

SEQ ID NO: 18 - protein, 16 aa, unidentified
KRPAATKKAGQAKKKK

SEQ ID NO: 19 - protein, 9 aa, unidentified
PAAKRVKLD

SEQ ID NO: 20 - protein, 11 aa, unidentified
RQRRNELKRSP

SEQ ID NO: 21 - protein, 38 aa, Homo sapiens
NQSSNFGPMKGGNFGGRSSGPYGGGGQYFAKPRNQGGY

SEQ ID NO: 22 - protein, 42 aa, unidentified
RMRIZFKNKGKDTAELRRRRVEVSVELRKAKKDEQILKRRNV

SEQ ID NO: 23 - protein, 8 aa, unidentified
VSRKRPRP

SEQ ID NO: 24 - protein, 8 aa, unidentified
PPKKARED

SEQ ID NO: 25 - protein, 8 aa, Homo sapiens
PQPKKKPL

SEQ ID NO: 26 - protein, 12 aa, Mus musculus
SALIKKKKKMAP

SEQ ID NO: 27 - protein, 5 aa, Influenza A virus
DRLRR

SEQ ID NO: 28 - protein, 7 aa, Influenza A virus
PKQKKRK

SEQ ID NO: 29 - protein, 10 aa, Hepacivirus D
RKLKKKIKKL

SEQ ID NO: 30 - protein, 10 aa, Mus musculus
REKKKFLKRR

SEQ ID NO: 31 - protein, 20 aa, Homo sapiens
KRKGDEVDGVDEVAKKKSKK

SEQ ID NO: 32 - protein, 17 aa, Homo sapiens
RKCLQAGMNLEARKTKK

SEQ ID NO: 33 - DNA, 8180 bp, synthetic construct
  source 1..8180: mol_type = other DNA
ggtcaagtgcctaagctcactcgagggtctcgtgagcgagcgagcgcgcagagagggagtggccaagctgaggctccatcactaggggttccttgtagttaatgattaacccgccatgctacttatcgcggccgcgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccacggtacccactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgaagaggtaagggtttaagggatggttggttggtggggtattaatgtttaattacctggagcacctgcctgaaatcactttttttcaggttggtttaaacgccgccaccatgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaatagcaggcaccccagcaccaggcagaagcagttcaatgccaccaccatcccagagaataccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactgattaattaagagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctctagagcatggctacgtagataagtagcatggcgggttaatcattaactacacctgcaggaggaacccctagtgatggagttggccactccctctctgcgcgctcgctcgctcactcgagaccaccggtgagcttaggcacttgaccggcaagcccttgtggacgtctactcccagtgtccacatggttaggaccgaggggctttgggtttagacactggcgacttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagaccgcgagaccttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacaccgcacatttccccgaaaagtgccacctgacgtctttcgtcagatcaccgggttgttccatatcatcgtgtccacaagggcttgcc

SEQ ID NO: 34 - DNA, 6206 bp, synthetic construct
  source 1..6206: mol_type = other DNA
ctgaggccgcccgggcaaagcccgggcgtcgggcgacctttggtcgcccggcctcagtgagcgagcgagcgcgcagagagggagtggccaagctgaggctccatcactaggggttccttgtagttaatgattaacccgccatgctacttatcgcggccgcgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccacggtacccactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgaagaggtaagggtttaagggatggttggttggtggggtattaatgtttaattacctggagcacctgcctgaaatcactttttttcaggttggtttaaacgccgccaccatgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaatagcaggcaccccagcaccaggcagaagcagttcaatgccaccaccatcccagagaataccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactgattaattaagagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctctagagcatggctacgtagataagtagcatggcgggttaatcattaactacacctgcaggaggaacccctagtgatggagttggccactccctctctgcgcgctcgctcgctcactgaggccgcccgggcaaagcccgggcgtcgggcgacctttggtcgcccggcctc

SEQ ID NO: 35 - DNA, 6206 bp, synthetic construct
  source 1..6206: mol_type = other DNA
  misc_difference 57: note = a, c, t, g, unknown or other
ctgaggccgcccgggcaaagcccgggcgtcgggcgacctttggtcgcccggcctcantgagcgagcgagcgcgcagagagggagtggccaagctgaggctccatcactaggggttccttgtagttaatgattaacccgccatgctacttatcgcggccgcgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccacggtacccactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgaagaggtaagggtttaagggatggttggttggtggggtattaatgtttaattacctggagcacctgcctgaaatcactttttttcaggttggtttaaacgccgccaccatgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaatagcaggcaccccagcaccaggcagaagcagttcaatgccaccaccatcccagagaataccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactgattaattaagagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctctagagcatggctacgtagataagtagcatggcgggttaatcattaactacacctgcaggaggaacccctagtgatggagttggccactccctctctgcgcgctcgctcgctcactgaggccgcccgggcaaagcccgggcgtcgggcgacctttggtcgcccggcctc

SEQ ID NO: 36 - DNA, 6207 bp, synthetic construct
  source 1..6207: mol_type = other DNA
  modified_base 6206: mod_base = OTHER; note = Inosine
gaggccgcccgggcaaagcccgggcgtcgggcgacctttggtcgcccggcctcagtgagcgagcgagcgcgcagagagggagtggccaactccatcactaggggttcctcctgcaggtgtagttaatgattaacccgccatgctacttatctacgtagccatgctctagagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagacaacaccacggaattatcagtgcccagcaacctagcccctgtccagcagcgggcaaggcaggcggcgatgagttctgccgtggcgatcgggagggggaaagcgaaagtcccagaaaggagttgacaggtggtggcaatgccccagccagtgggggttgcgtcagcaaacacagagcacaccacgccacgttgacggacaacgggccacaactcctctaaaagagacagcaaccaggatttatacaaggaggagaaaacgaaagccgtacgggaagcaatagctagatacagaggctataaagcagcatatccacacagcgtaaaaggagcaacatagttaagaatatcagtcaatctttcacaaattttgtaatccagaggttgattaacaggaacagagcgtaaataacgggaaagtttcttaacatgtttgtcttgtggcaatacacctgaactagtaattacatatccctaaaaatgtaaatgattgccccaccattttgttttattaacatttaaatgtatacccaaatcaagaaaaacagaacaaatatgggaataaatggcggtaagatgctcttaattaatcagtacaggtcctgggcctcacagcccagcacctccatcctcagggcaatctggtgcacccagctctgggggtgaatcctcaggtatctggtcagcaggggggggtccaggctgttcaccacaggggtgaagctgtcctggttgccctggaacaccttcaccttgccattctggaagaacagggtccactggtggccatcctggctgctgctgatcaggaactccttcacatacatgctggtcagcaggctcttcaccccctgggtggtcaccccagtcaccttcatggtcttctggaagtccacctgcagccactccttggggttgttgacctggggcctccaggcattgctcctgccctgcaggtgcagcctggccttgctggggctccaggtggcaaacatgttggtgaagtagctgctggcagtgatctgggcatcagagatggccttgctctccatgcccaggggcatgctgcagctgttcaggtcacagcccatcagctccatcctcagggtgctcctgatgctgtagtgggtggggtgcagcctgatgtatctggcaatgatgggggggttgaagatgttgtgcttgatgccagagctgtccacattgccaaagaacaccatcagggtgccagtgctgttgcccctgtaggtctgccacttcttgccatccaggctgtacatgatgatgaactggctgatgtacaggctgctgaacttctgcctggccccctgggtcttgatgccatggatgatcatgggggccagcaggtccaccttgatccagctgaagggctccttggtgctccaggcattgatgctgccagagtagtgcagcctggccagcttgggggcccactggccatactggccagaggcagtgatctggaagtccctgatgtggccagaggccatgcccaggggggtctggcacttgttgctgtacaccaggaacagggtgctcatgccagcatgcaggtgctccccaatcaggcactccaccctccagatgccagccttgctgggcagcatctccacagtctcaaacaccccagggtacaggttgtacagggccatcttgtactcctccttcttcctcacagtgaacacatggccagagaagtggatgctgtggatgttctcattgctgcccatgctcagcaggtaccacctgatcctctggtcctgggccatcaccaggccaggcagggtgtccatgatgtagccattgatggcatggaacctgtagttctccttgaaggtggggtcctccatctggatgttgcagggggccctgcagttcctctccatgttctcagtgaagtaccagctcttggtttcatcaaagatggtgaagaacagggcaaactcctgcacagtcacctgcctgccatgggcagggttcagggtgttggtgtggcacaccagcagggggccaatcaggccagagtgcacatccttctccaggtccacatcagagaagtaggcccaggccttgcagtcaaactcatccttggtgggggccatgtggtgctgcaccttccagaagtaggtcttggtttcattgggcttcacaaagttcttcctgggctcagccccctgcctctggtcctcctcatagctgatcaggctgctgtagaagctgtagggcctgctggcctggttcctgaaggtcaccatgatgttgtcctccacctcagccctgatgtaggggcccagcaggcccaggtgctcattcagctcccctctgtacaggggctgggtgaagctgccatcagtgaactcctggaacaccaccttcttgaactggggcacagagccagactgggccctgttcctcagcacatgggggctgctgctcatgccatagtcccacagcctctccacagcagcaatgaagtagtgcctggtcttcttctggaagctcctggggctctggttctcgtcctcgtcgtagatgtcaaagtcctccttcttcatctccacagagatggtgtcatcatagtcaatctcctcctggtcagactgcagggtggtattctctgggatggtggtggcattgaactgcttctgcctggtgctggggtgcctgctattctggctgaagctcctgggctcaatggcattgttcttgctcagcaggtaggcagagatgtcctcatagctgtcctcatagtagtccccagtgttcttgtcacagctggagactttcagcagggcagtcatgcccctgttcctgaagtcagagttgtggcagcccagaatccacaggccagggttctccatgctcatgaacacagtctccccagagaaggggaacagggtcagggtgtcctcatacaccatcttgtgcttgaaggtgtagccagagaagaacacagacaggaagtcagtctgggccccaatgctcaggatgtaccagtaggccacctcatgcaggcacacagacagctgcaggctgtcaaacacatagccattgatgctgtgcatgatgttgctggcctggaactcagggtcctccagctgcaccccagcagggttgggcaggaacctctggatgttctcagtcaggtaccagctcctgttctcatcaaacacagagaacaggatcacattcctcttgtcagacatgatctggttgcccctctggtccacagactccttgtagcagatcagcagggggccaatcaggccagaggccaggtccctctccatgttcacaaagctgctgtagtatctggtcaggcacctggggtcagacttggtggggccatcctccacagtcacagtccacttgtacttgaagatctccccaggcaggatggggaagtccttcaggtgcttcacccccttgggcagcctcctgctgtacaggggcctcacatcagtgatgccatgggggtagatgttgtagggcctgctggcctggttcttgaagatgatcagcagggtgtcccccacctccccatacagcagggggcccaggatgccagactcatgctggatggcctccctggtcttgaaggtttcatcagtgtaggccatgaacctgaccttcttgtacttcctgccaatcctctgggggccattgttcaggtactggctcttgtagctcctgtcatcaggggccagcaccaggggggcatagtcccagtcctcctcctcagcagcaatgtagtgcacccaggtcttggggtgcttcttggccacagacctgatctggatgaagctggggctgttgtcatcatcaaacctcaccacatccatctcagagtcagtcaggtcatcatcatagtcctcagcctcctcattgttcttcatcctcagctggggctcctcagggcagctgtccaccttcacataggcctccatgccatcatgctggtggctgctgatgtggcagaacagcaggaactggcccaggtccatcagcagggtctgggcagtcaggaaggtgatggggctgatctccaggctggcctgcctgtggttcctgaccaggaaggtgtggccctccaggaagatgctgtgcacctcaggggtggtgcccatgccaatcacatgccagtacacagacttcctgtggcagccaatcaggccaggcaggctcctgttcacatagccattcacagtgtgcatcttgggccaggccctggcagaggcagcatccctgtcctgcatcaggctgttcttggtttcagagtgccagctcttgccctcatcaaacacagcaaacagcaggatgaacttgtgcagggtctgggtcttctccttggccaggctgccctccctgcacaccagcagggccccaatcaggccagagttcaggtccttcaccaggtccacatggctcaggtagctgtaggtcaggcacagggggtcagaggccatggggccattctccttcagcacctgccacacataggtgtggctgcccccagggaacaccttgtcatcctccttctccctctggctggtctggtcatcatactcagccccctcagaggccttccagtagctcacccccacagcatgcaggctcacagggtggctggccatgttcttcagggtgatcaccacagtgtcatacacctcagcctggatggtggggcccagcaggcccatccaggggggcctgggcttggcaatgttgaacaggtggtcagtgaactccacaaacagggtcttcttgtacaccacagaggtgttgaaggggaagctcttgggcactctgggggggaacctggcatccacaggcagctcccccaggtcagactgcatgtagtcccagctcagctccacagcccccaggtagtatctcctggtggcagagaagcagaacctcagcaggcacaggaagaagcaggtgctcagctcaatctgcatggtggcggcgtttaaaccaacctgaaaaaaagtgatttcaggcaggtgctccaggtaattaaacattaataccccaccaaccaaccatcccttaaacccttacctcttcaggagcttgtggatctgtgtgacggcttctcctggtgaaggggcttttataccccctccttccaacccaggctgctgatccctgccaagctgactccaaacctgctgattctgattattgacttagtcaacaaaaggagaataagtaacctacacaaatatgaaccttgcctagggagattagagtatcggaacactcgctctacgaaatgtgcagacagacggggatcctctagagctacctgctgatcgcccggcccctgttcaaacatgtcctaatactctgtctctgcaagggtcatcagtagttttccatcttactcaacatcctcccagtgggtaccgtggacttagcccctgtttgctcctccgataactggggtgaccttggttaatattcaccagcagcctcccccggtggacttagcccctgtttgctcctccgataactggggtgaccttggttaatattcaccagcagcctcccccggtggacttagcccctgtttgctcctccgataactggggtgaccttggttaatattcaccagcagcctcccccgcggccgcgataagtagcatggcgggttaatcattaactacaaggaacccctagtgatggagcctcagcttggccactccctctctgcgcgctcgctcgctcactgaggccgcccgggcaaagcccgggcgtcgggcgacctttggtcgcccggcctcant

SEQ ID NO: 37 - DNA, 6206 bp, synthetic construct
  source 1..6206: mol_type = other DNA
  misc_difference 56: note = a, c, t, g, unknown or other
ctgaggccgcccgggcaaagcccgggcgtcgggcgacctttggtcgcccggcctcngtgagcgagcgagcgcgcagagagggagtggccaagctgaggctccatcactaggggttccttgtagttaatgattaacccgccatgctacttatcgcggccgcgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccacggtacccactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgaagaggtaagggtttaagggatggttggttggtggggtattaatgtttaattacctggagcacctgcctgaaatcactttttttcaggttggtttaaacgccgccaccatgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaatagcaggcaccccagcaccaggcagaagcagttcaatgccaccaccatcccagagaataccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactgattaattaagagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctctagagcatggctacgtagataagtagcatggcgggttaatcattaactacacctgcaggaggaacccctagtgatggagttggccactccctctctgcgcgctcgctcgctcactgaggccgcccgggcaaagcccgggcgtcgggcgacctttggtcgcccggcctc

SEQ ID NO: 38 - DNA, 6206 bp, synthetic construct
  source 1..6206: mol_type = other DNA
  modified_base 6205: mod_base = OTHER; note = Inosine
gaggccgcccgggcaaagcccgggcgtcgggcgacctttggtcgcccggcctcagtgagcgagcgagcgcgcagagagggagtggccaactccatcactaggggttcctcctgcaggtgtagttaatgattaacccgccatgctacttatctacgtagccatgctctagagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagacaacaccacggaattatcagtgcccagcaacctagcccctgtccagcagcgggcaaggcaggcggcgatgagttctgccgtggcgatcgggagggggaaagcgaaagtcccagaaaggagttgacaggtggtggcaatgccccagccagtgggggttgcgtcagcaaacacagagcacaccacgccacgttgacggacaacgggccacaactcctctaaaagagacagcaaccaggatttatacaaggaggagaaaacgaaagccgtacgggaagcaatagctagatacagaggctataaagcagcatatccacacagcgtaaaaggagcaacatagttaagaatatcagtcaatctttcacaaattttgtaatccagaggttgattaacaggaacagagcgtaaataacgggaaagtttcttaacatgtttgtcttgtggcaatacacctgaactagtaattacatatccctaaaaatgtaaatgattgccccaccattttgttttattaacatttaaatgtatacccaaatcaagaaaaacagaacaaatatgggaataaatggcggtaagatgctcttaattaatcagtacaggtcctgggcctcacagcccagcacctccatcctcagggcaatctggtgcacccagctctgggggtgaatcctcaggtatctggtcagcaggggggggtccaggctgttcaccacaggggtgaagctgtcctggttgccctggaacaccttcaccttgccattctggaagaacagggtccactggtggccatcctggctgctgctgatcaggaactccttcacatacatgctggtcagcaggctcttcaccccctgggtggtcaccccagtcaccttcatggtcttctggaagtccacctgcagccactccttggggttgttgacctggggcctccaggcattgctcctgccctgcaggtgcagcctggccttgctggggctccaggtggcaaacatgttggtgaagtagctgctggcagtgatctgggcatcagagatggccttgctctccatgcccaggggcatgctgcagctgttcaggtcacagcccatcagctccatcctcagggtgctcctgatgctgtagtgggtggggtgcagcctgatgtatctggcaatgatgggggggttgaagatgttgtgcttgatgccagagctgtccacattgccaaagaacaccatcagggtgccagtgctgttgcccctgtaggtctgccacttcttgccatccaggctgtacatgatgatgaactggctgatgtacaggctgctgaacttctgcctggccccctgggtcttgatgccatggatgatcatgggggccagcaggtccaccttgatccagctgaagggctccttggtgctccaggcattgatgctgccagagtagtgcagcctggccagcttgggggcccactggccatactggccagaggcagtgatctggaagtccctgatgtggccagaggccatgcccaggggggtctggcacttgttgctgtacaccaggaacagggtgctcatgccagcatgcaggtgctccccaatcaggcactccaccctccagatgccagccttgctgggcagcatctccacagtctcaaacaccccagggtacaggttgtacagggccatcttgtactcctccttcttcctcacagtgaacacatggccagagaagtggatgctgtggatgttctcattgctgcccatgctcagcaggtaccacctgatcctctggtcctgggccatcaccaggccaggcagggtgtccatgatgtagccattgatggcatggaacctgtagttctccttgaaggtggggtcctccatctggatgttgcagggggccctgcagttcctctccatgttctcagtgaagtaccagctcttggtttcatcaaagatggtgaagaacagggcaaactcctgcacagtcacctgcctgccatgggcagggttcagggtgttggtgtggcacaccagcagggggccaatcaggccagagtgcacatccttctccaggtccacatcagagaagtaggcccaggccttgcagtcaaactcatccttggtgggggccatgtggtgctgcaccttccagaagtaggtcttggtttcattgggcttcacaaagttcttcctgggctcagccccctgcctctggtcctcctcatagctgatcaggctgctgtagaagctgtagggcctgctggcctggttcctgaaggtcaccatgatgttgtcctccacctcagccctgatgtaggggcccagcaggcccaggtgctcattcagctcccctctgtacaggggctgggtgaagctgccatcagtgaactcctggaacaccaccttcttgaactggggcacagagccagactgggccctgttcctcagcacatgggggctgctgctcatgccatagtcccacagcctctccacagcagcaatgaagtagtgcctggtcttcttctggaagctcctggggctctggttctcgtcctcgtcgtagatgtcaaagtcctccttcttcatctccacagagatggtgtcatcatagtcaatctcctcctggtcagactgcagggtggtattctctgggatggtggtggcattgaactgcttctgcctggtgctggggtgcctgctattctggctgaagctcctgggctcaatggcattgttcttgctcagcaggtaggcagagatgtcctcatagctgtcctcatagtagtccccagtgttcttgtcacagctggagactttcagcagggcagtcatgcccctgttcctgaagtcagagttgtggcagcccagaatccacaggccagggttctccatgctcatgaacacagtctccccagagaaggggaacagggtcagggtgtcctcatacaccatcttgtgcttgaaggtgtagccagagaagaacacagacaggaagtcagtctgggccccaatgctcaggatgtaccagtaggccacctcatgcaggcacacagacagctgcaggctgtcaaacacatagccattgatgctgtgcatgatgttgctggcctggaactcagggtcctccagctgcaccccagcagggttgggcaggaacctctggatgttctcagtcaggtaccagctcctgttctcatcaaacacagagaacaggatcacattcctcttgtcagacatgatctggttgcccctctggtccacagactccttgtagcagatcagcagggggccaatcaggccagaggccaggtccctctccatgttcacaaagctgctgtagtatctggtcaggcacctggggtcagacttggtggggccatcctccacagtcacagtccacttgtacttgaagatctccccaggcaggatggggaagtccttcaggtgcttcacccccttgggcagcctcctgctgtacaggggcctcacatcagtgatgccatgggggtagatgttgtagggcctgctggcctggttcttgaagatgatcagcagggtgtcccccacctccccatacagcagggggcccaggatgccagactcatgctggatggcctccctggtcttgaaggtttcatcagtgtaggccatgaacctgaccttcttgtacttcctgccaatcctctgggggccattgttcaggtactggctcttgtagctcctgtcatcaggggccagcaccaggggggcatagtcccagtcctcctcctcagcagcaatgtagtgcacccaggtcttggggtgcttcttggccacagacctgatctggatgaagctggggctgttgtcatcatcaaacctcaccacatccatctcagagtcagtcaggtcatcatcatagtcctcagcctcctcattgttcttcatcctcagctggggctcctcagggcagctgtccaccttcacataggcctccatgccatcatgctggtggctgctgatgtggcagaacagcaggaactggcccaggtccatcagcagggtctgggcagtcaggaaggtgatggggctgatctccaggctggcctgcctgtggttcctgaccaggaaggtgtggccctccaggaagatgctgtgcacctcaggggtggtgcccatgccaatcacatgccagtacacagacttcctgtggcagccaatcaggccaggcaggctcctgttcacatagccattcacagtgtgcatcttgggccaggccctggcagaggcagcatccctgtcctgcatcaggctgttcttggtttcagagtgccagctcttgccctcatcaaacacagcaaacagcaggatgaacttgtgcagggtctgggtcttctccttggccaggctgccctccctgcacaccagcagggccccaatcaggccagagttcaggtccttcaccaggtccacatggctcaggtagctgtaggtcaggcacagggggtcagaggccatggggccattctccttcagcacctgccacacataggtgtggctgcccccagggaacaccttgtcatcctccttctccctctggctggtctggtcatcatactcagccccctcagaggccttccagtagctcacccccacagcatgcaggctcacagggtggctggccatgttcttcagggtgatcaccacagtgtcatacacctcagcctggatggtggggcccagcaggcccatccaggggggcctgggcttggcaatgttgaacaggtggtcagtgaactccacaaacagggtcttcttgtacaccacagaggtgttgaaggggaagctcttgggcactctgggggggaacctggcatccacaggcagctcccccaggtcagactgcatgtagtcccagctcagctccacagcccccaggtagtatctcctggtggcagagaagcagaacctcagcaggcacaggaagaagcaggtgctcagctcaatctgcatggtggcggcgtttaaaccaacctgaaaaaaagtgatttcaggcaggtgctccaggtaattaaacattaataccccaccaaccaaccatcccttaaacccttacctcttcaggagcttgtggatctgtgtgacggcttctcctggtgaaggggcttttataccccctccttccaacccaggctgctgatccctgccaagctgactccaaacctgctgattctgattattgacttagtcaacaaaaggagaataagtaacctacacaaatatgaaccttgcctagggagattagagtatcggaacactcgctctacgaaatgtgcagacagacggggatcctctagagctacctgctgatcgcccggcccctgttcaaacatgtcctaatactctgtctctgcaagggtcatcagtagttttccatcttactcaacatcctcccagtgggtaccgtggacttagcccctgtttgctcctccgataactggggtgaccttggttaatattcaccagcagcctcccccggtggacttagcccctgtttgctcctccgataactggggtgaccttggttaatattcaccagcagcctcccccggtggacttagcccctgtttgctcctccgataactggggtgaccttggttaatattcaccagcagcctcccccgcggccgcgataagtagcatggcgggttaatcattaactacaaggaacccctagtgatggagcctcagcttggccactccctctctgcgcgctcgctcgctcactgaggccgcccgggcaaagcccgggcgtcgggcgacctttggtcgcccggcctcng

SEQ ID NO: 39 - DNA, 6206 bp, synthetic construct
  source 1..6206: mol_type = other DNA
  misc_difference 53: note = a, c, t, g, unknown or other
ctgaggccgcccgggcaaagcccgggcgtcgggcgacctttggtcgcccggcntcagtgagcgagcgagcgcgcagagagggagtggccaagctgaggctccatcactaggggttccttgtagttaatgattaacccgccatgctacttatcgcggccgcgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccacggtacccactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgaagaggtaagggtttaagggatggttggttggtggggtattaatgtttaattacctggagcacctgcctgaaatcactttttttcaggttggtttaaacgccgccaccatgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaatagcaggcaccccagcaccaggcagaagcagttcaatgccaccaccatcccagagaataccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactgattaattaagagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctctagagcatggctacgtagataagtagcatggcgggttaatcattaactacacctgcaggaggaacccctagtgatggagttggccactccctctctgcgcgctcgctcgctcactgaggccgcccgggcaaagcccgggcgtcgggcgacctttggtcgcccggcctc

SEQ ID NO: 40 - DNA, 6203 bp, synthetic construct
  source 1..6203: mol_type = other DNA
  modified_base 6202: mod_base = OTHER; note = Inosine
gaggccgcccgggcaaagcccgggcgtcgggcgacctttggtcgcccggcctcagtgagcgagcgagcgcgcagagagggagtggccaactccatcactaggggttcctcctgcaggtgtagttaatgattaacccgccatgctacttatctacgtagccatgctctagagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagacaacaccacggaattatcagtgcccagcaacctagcccctgtccagcagcgggcaaggcaggcggcgatgagttctgccgtggcgatcgggagggggaaagcgaaagtcccagaaaggagttgacaggtggtggcaatgccccagccagtgggggttgcgtcagcaaacacagagcacaccacgccacgttgacggacaacgggccacaactcctctaaaagagacagcaaccaggatttatacaaggaggagaaaacgaaagccgtacgggaagcaatagctagatacagaggctataaagcagcatatccacacagcgtaaaaggagcaacatagttaagaatatcagtcaatctttcacaaattttgtaatccagaggttgattaacaggaacagagcgtaaataacgggaaagtttcttaacatgtttgtcttgtggcaatacacctgaactagtaattacatatccctaaaaatgtaaatgattgccccaccattttgttttattaacatttaaatgtatacccaaatcaagaaaaacagaacaaatatgggaataaatggcggtaagatgctcttaattaatcagtacaggtcctgggcctcacagcccagcacctccatcctcagggcaatctggtgcacccagctctgggggtgaatcctcaggtatctggtcagcaggggggggtccaggctgttcaccacaggggtgaagctgtcctggttgccctggaacaccttcaccttgccattctggaagaacagggtccactggtggccatcctggctgctgctgatcaggaactccttcacatacatgctggtcagcaggctcttcaccccctgggtggtcaccccagtcaccttcatggtcttctggaagtccacctgcagccactccttggggttgttgacctggggcctccaggcattgctcctgccctgcaggtgcagcctggccttgctggggctccaggtggcaaacatgttggtgaagtagctgctggcagtgatctgggcatcagagatggccttgctctccatgcccaggggcatgctgcagctgttcaggtcacagcccatcagctccatcctcagggtgctcctgatgctgtagtgggtggggtgcagcctgatgtatctggcaatgatgggggggttgaagatgttgtgcttgatgccagagctgtccacattgccaaagaacaccatcagggtgccagtgctgttgcccctgtaggtctgccacttcttgccatccaggctgtacatgatgatgaactggctgatgtacaggctgctgaacttctgcctggccccctgggtcttgatgccatggatgatcatgggggccagcaggtccaccttgatccagctgaagggctccttggtgctccaggcattgatgctgccagagtagtgcagcctggccagcttgggggcccactggccatactggccagaggcagtgatctggaagtccctgatgtggccagaggccatgcccaggggggtctggcacttgttgctgtacaccaggaacagggtgctcatgccagcatgcaggtgctccccaatcaggcactccaccctccagatgccagccttgctgggcagcatctccacagtctcaaacaccccagggtacaggttgtacagggccatcttgtactcctccttcttcctcacagtgaacacatggccagagaagtggatgctgtggatgttctcattgctgcccatgctcagcaggtaccacctgatcctctggtcctgggccatcaccaggccaggcagggtgtccatgatgtagccattgatggcatggaacctgtagttctccttgaaggtggggtcctccatctggatgttgcagggggccctgcagttcctctccatgttctcagtgaagtaccagctcttggtttcatcaaagatggtgaagaacagggcaaactcctgcacagtcacctgcctgccatgggcagggttcagggtgttggtgtggcacaccagcagggggccaatcaggccagagtgcacatccttctccaggtccacatcagagaagtaggcccaggccttgcagtcaaactcatccttggtgggggccatgtggtgctgcaccttccagaagtaggtcttggtttcattgggcttcacaaagttcttcctgggctcagccccctgcctctggtcctcctcatagctgatcaggctgctgtagaagctgtagggcctgctggcctggttcctgaaggtcaccatgatgttgtcctccacctcagccctgatgtaggggcccagcaggcccaggtgctcattcagctcccctctgtacaggggctgggtgaagctgccatcagtgaactcctggaacaccaccttcttgaactggggcacagagccagactgggccctgttcctcagcacatgggggctgctgctcatgccatagtcccacagcctctccacagcagcaatgaagtagtgcctggtcttcttctggaagctcctggggctctggttctcgtcctcgtcgtagatgtcaaagtcctccttcttcatctccacagagatggtgtcatcatagtcaatctcctcctggtcagactgcagggtggtattctctgggatggtggtggcattgaactgcttctgcctggtgctggggtgcctgctattctggctgaagctcctgggctcaatggcattgttcttgctcagcaggtaggcagagatgtcctcatagctgtcctcatagtagtccccagtgttcttgtcacagctggagactttcagcagggcagtcatgcccctgttcctgaagtcagagttgtggcagcccagaatccacaggccagggttctccatgctcatgaacacagtctccccagagaaggggaacagggtcagggtgtcctcatacaccatcttgtgcttgaaggtgtagccagagaagaacacagacaggaagtcagtctgggccccaatgctcaggatgtaccagtaggccacctcatgcaggcacacagacagctgcaggctgtcaaacacatagccattgatgctgtgcatgatgttgctggcctggaactcagggtcctccagctgcaccccagcagggttgggcaggaacctctggatgttctcagtcaggtaccagctcctgttctcatcaaacacagagaacaggatcacattcctcttgtcagacatgatctggttgcccctctggtccacagactccttgtagcagatcagcagggggccaatcaggccagaggccaggtccctctccatgttcacaaagctgctgtagtatctggtcaggcacctggggtcagacttggtggggccatcctccacagtcacagtccacttgtacttgaagatctccccaggcaggatggggaagtccttcaggtgcttcacccccttgggcagcctcctgctgtacaggggcctcacatcagtgatgccatgggggtagatgttgtagggcctgctggcctggttcttgaagatgatcagcagggtgtcccccacctccccatacagcagggggcccaggatgccagactcatgctggatggcctccctggtcttgaaggtttcatcagtgtaggccatgaacctgaccttcttgtacttcctgccaatcctctgggggccattgttcaggtactggctcttgtagctcctgtcatcaggggccagcaccaggggggcatagtcccagtcctcctcctcagcagcaatgtagtgcacccaggtcttggggtgcttcttggccacagacctgatctggatgaagctggggctgttgtcatcatcaaacctcaccacatccatctcagagtcagtcaggtcatcatcatagtcctcagcctcctcattgttcttcatcctcagctggggctcctcagggcagctgtccaccttcacataggcctccatgccatcatgctggtggctgctgatgtggcagaacagcaggaactggcccaggtccatcagcagggtctgggcagtcaggaaggtgatggggctgatctccaggctggcctgcctgtggttcctgaccaggaaggtgtggccctccaggaagatgctgtgcacctcaggggtggtgcccatgccaatcacatgccagtacacagacttcctgtggcagccaatcaggccaggcaggctcctgttcacatagccattcacagtgtgcatcttgggccaggccctggcagaggcagcatccctgtcctgcatcaggctgttcttggtttcagagtgccagctcttgccctcatcaaacacagcaaacagcaggatgaacttgtgcagggtctgggtcttctccttggccaggctgccctccctgcacaccagcagggccccaatcaggccagagttcaggtccttcaccaggtccacatggctcaggtagctgtaggtcaggcacagggggtcagaggccatggggccattctccttcagcacctgccacacataggtgtggctgcccccagggaacaccttgtcatcctccttctccctctggctggtctggtcatcatactcagccccctcagaggccttccagtagctcacccccacagcatgcaggctcacagggtggctggccatgttcttcagggtgatcaccacagtgtcatacacctcagcctggatggtggggcccagcaggcccatccaggggggcctgggcttggcaatgttgaacaggtggtcagtgaactccacaaacagggtcttcttgtacaccacagaggtgttgaaggggaagctcttgggcactctgggggggaacctggcatccacaggcagctcccccaggtcagactgcatgtagtcccagctcagctccacagcccccaggtagtatctcctggtggcagagaagcagaacctcagcaggcacaggaagaagcaggtgctcagctcaatctgcatggtggcggcgtttaaaccaacctgaaaaaaagtgatttcaggcaggtgctccaggtaattaaacattaataccccaccaaccaaccatcccttaaacccttacctcttcaggagcttgtggatctgtgtgacggcttctcctggtgaaggggcttttataccccctccttccaacccaggctgctgatccctgccaagctgactccaaacctgctgattctgattattgacttagtcaacaaaaggagaataagtaacctacacaaatatgaaccttgcctagggagattagagtatcggaacactcgctctacgaaatgtgcagacagacggggatcctctagagctacctgctgatcgcccggcccctgttcaaacatgtcctaatactctgtctctgcaagggtcatcagtagttttccatcttactcaacatcctcccagtgggtaccgtggacttagcccctgtttgctcctccgataactggggtgaccttggttaatattcaccagcagcctcccccggtggacttagcccctgtttgctcctccgataactggggtgaccttggttaatattcaccagcagcctcccccggtggacttagcccctgtttgctcctccgataactggggtgaccttggttaatattcaccagcagcctcccccgcggccgcgataagtagcatggcgggttaatcattaactacaaggaacccctagtgatggagcctcagcttggccactccctctctgcgcgctcgctcgctcactgaggccgcccgggcaaagcccgggcgtcgggcgacctttggtcgcccggcnt

SEQ ID NO: 41 - DNA, 6214 bp, synthetic construct
  source 1..6214: mol_type = other DNA
  misc_difference 57: note = a, c, t, g, unknown or other
ctgaggccgcccgggcaaagcccgggcgtcgggcgacctttggtcgcccggcctcantgagcgagcgagcgcgcagagagggagtggccaagctgaggctccatcactaggggttccttgtagttaatgattaacccgccatgctacttatcgcggccgcgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccacggtacccactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgaagaggtaagggtttaagggatggttggttggtggggtattaatgtttaattacctggagcacctgcctgaaatcactttttttcaggttggtttaaacgccgccaccatgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaatagcaggcaccccagcaccaggcagaagcagttcaatgccaccaccatcccagagaataccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactgattaattaagagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctctagagcatggctacgtagataagtagcatggcgggttaatcattaactacacctgcaggaggaacccctagtgatggagttggccactccctctctgcgcgctcgctcgctcactgagggaggccgcccgggcaaagcccgggcgtcgggcgacctttggtcgcccggcctccctc

SEQ ID NO: 42 - DNA, 6215 bp, synthetic construct
  source 1..6215: mol_type = other DNA
  modified_base 6214: mod_base = OTHER; note = Inosine
gaggccgcccgggcaaagcccgggcgtcgggcgacctttggtcgcccggcctccctccctcagtgagcgagcgagcgcgcagagagggagtggccaactccatcactaggggttcctcctgcaggtgtagttaatgattaacccgccatgctacttatctacgtagccatgctctagagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagacaacaccacggaattatcagtgcccagcaacctagcccctgtccagcagcgggcaaggcaggcggcgatgagttctgccgtggcgatcgggagggggaaagcgaaagtcccagaaaggagttgacaggtggtggcaatgccccagccagtgggggttgcgtcagcaaacacagagcacaccacgccacgttgacggacaacgggccacaactcctctaaaagagacagcaaccaggatttatacaaggaggagaaaacgaaagccgtacgggaagcaatagctagatacagaggctataaagcagcatatccacacagcgtaaaaggagcaacatagttaagaatatcagtcaatctttcacaaattttgtaatccagaggttgattaacaggaacagagcgtaaataacgggaaagtttcttaacatgtttgtcttgtggcaatacacctgaactagtaattacatatccctaaaaatgtaaatgattgccccaccattttgttttattaacatttaaatgtatacccaaatcaagaaaaacagaacaaatatgggaataaatggcggtaagatgctcttaattaatcagtacaggtcctgggcctcacagcccagcacctccatcctcagggcaatctggtgcacccagctctgggggtgaatcctcaggtatctggtcagcaggggggggtccaggctgttcaccacaggggtgaagctgtcctggttgccctggaacaccttcaccttgccattctggaagaacagggtccactggtggccatcctggctgctgctgatcaggaactccttcacatacatgctggtcagcaggctcttcaccccctgggtggtcaccccagtcaccttcatggtcttctggaagtccacctgcagccactccttggggttgttgacctggggcctccaggcattgctcctgccctgcaggtgcagcctggccttgctggggctccaggtggcaaacatgttggtgaagtagctgctggcagtgatctgggcatcagagatggccttgctctccatgcccaggggcatgctgcagctgttcaggtcacagcccatcagctccatcctcagggtgctcctgatgctgtagtgggtggggtgcagcctgatgtatctggcaatgatgggggggttgaagatgttgtgcttgatgccagagctgtccacattgccaaagaacaccatcagggtgccagtgctgttgcccctgtaggtctgccacttcttgccatccaggctgtacatgatgatgaactggctgatgtacaggctgctgaacttctgcctggccccctgggtcttgatgccatggatgatcatgggggccagcaggtccaccttgatccagctgaagggctccttggtgctccaggcattgatgctgccagagtagtgcagcctggccagcttgggggcccactggccatactggccagaggcagtgatctggaagtccctgatgtggccagaggccatgcccaggggggtctggcacttgttgctgtacaccaggaacagggtgctcatgccagcatgcaggtgctccccaatcaggcactccaccctccagatgccagccttgctgggcagcatctccacagtctcaaacaccccagggtacaggttgtacagggccatcttgtactcctccttcttcctcacagtgaacacatggccagagaagtggatgctgtggatgttctcattgctgcccatgctcagcaggtaccacctgatcctctggtcctgggccatcaccaggccaggcagggtgtccatgatgtagccattgatggcatggaacctgtagttctccttgaaggtggggtcctccatctggatgttgcagggggccctgcagttcctctccatgttctcagtgaagtaccagctcttggtttcatcaaagatggtgaagaacagggcaaactcctgcacagtcacctgcctgccatgggcagggttcagggtgttggtgtggcacaccagcagggggccaatcaggccagagtgcacatccttctccaggtccacatcagagaagtaggcccaggccttgcagtcaaactcatccttggtgggggccatgtggtgctgcaccttccagaagtaggtcttggtttcattgggcttcacaaagttcttcctgggctcagccccctgcctctggtcctcctcatagctgatcaggctgctgtagaagctgtagggcctgctggcctggttcctgaaggtcaccatgatgttgtcctccacctcagccctgatgtaggggcccagcaggcccaggtgctcattcagctcccctctgtacaggggctgggtgaagctgccatcagtgaactcctggaacaccaccttcttgaactggggcacagagccagactgggccctgttcctcagcacatgggggctgctgctcatgccatagtcccacagcctctccacagcagcaatgaagtagtgcctggtcttcttctggaagctcctggggctctggttctcgtcctcgtcgtagatgtcaaagtcctccttcttcatctccacagagatggtgtcatcatagtcaatctcctcctggtcagactgcagggtggtattctctgggatggtggtggcattgaactgcttctgcctggtgctggggtgcctgctattctggctgaagctcctgggctcaatggcattgttcttgctcagcaggtaggcagagatgtcctcatagctgtcctcatagtagtccccagtgttcttgtcacagctggagactttcagcagggcagtcatgcccctgttcctgaagtcagagttgtggcagcccagaatccacaggccagggttctccatgctcatgaacacagtctccccagagaaggggaacagggtcagggtgtcctcatacaccatcttgtgcttgaaggtgtagccagagaagaacacagacaggaagtcagtctgggccccaatgctcaggatgtaccagtaggccacctcatgcaggcacacagacagctgcaggctgtcaaacacatagccattgatgctgtgcatgatgttgctggcctggaactcagggtcctccagctgcaccccagcagggttgggcaggaacctctggatgttctcagtcaggtaccagctcctgttctcatcaaacacagagaacaggatcacattcctcttgtcagacatgatctggttgcccctctggtccacagactccttgtagcagatcagcagggggccaatcaggccagaggccaggtccctctccatgttcacaaagctgctgtagtatctggtcaggcacctggggtcagacttggtggggccatcctccacagtcacagtccacttgtacttgaagatctccccaggcaggatggggaagtccttcaggtgcttcacccccttgggcagcctcctgctgtacaggggcctcacatcagtgatgccatgggggtagatgttgtagggcctgctggcctggttcttgaagatgatcagcagggtgtcccccacctccccatacagcagggggcccaggatgccagactcatgctggatggcctccctggtcttgaaggtttcatcagtgtaggccatgaacctgaccttcttgtacttcctgccaatcctctgggggccattgttcaggtactggctcttgtagctcctgtcatcaggggccagcaccaggggggcatagtcccagtcctcctcctcagcagcaatgtagtgcacccaggtcttggggtgcttcttggccacagacctgatctggatgaagctggggctgttgtcatcatcaaacctcaccacatccatctcagagtcagtcaggtcatcatcatagtcctcagcctcctcattgttcttcatcctcagctggggctcctcagggcagctgtccaccttcacataggcctccatgccatcatgctggtggctgctgatgtggcagaacagcaggaactggcccaggtccatcagcagggtctgggcagtcaggaaggtgatggggctgatctccaggctggcctgcctgtggttcctgaccaggaaggtgtggccctccaggaagatgctgtgcacctcaggggtggtgcccatgccaatcacatgccagtacacagacttcctgtggcagccaatcaggccaggcaggctcctgttcacatagccattcacagtgtgcatcttgggccaggccctggcagaggcagcatccctgtcctgcatcaggctgttcttggtttcagagtgccagctcttgccctcatcaaacacagcaaacagcaggatgaacttgtgcagggtctgggtcttctccttggccaggctgccctccctgcacaccagcagggccccaatcaggccagagttcaggtccttcaccaggtccacatggctcaggtagctgtaggtcaggcacagggggtcagaggccatggggccattctccttcagcacctgccacacataggtgtggctgcccccagggaacaccttgtcatcctccttctccctctggctggtctggtcatcatactcagccccctcagaggccttccagtagctcacccccacagcatgcaggctcacagggtggctggccatgttcttcagggtgatcaccacagtgtcatacacctcagcctggatggtggggcccagcaggcccatccaggggggcctgggcttggcaatgttgaacaggtggtcagtgaactccacaaacagggtcttcttgtacaccacagaggtgttgaaggggaagctcttgggcactctgggggggaacctggcatccacaggcagctcccccaggtcagactgcatgtagtcccagctcagctccacagcccccaggtagtatctcctggtggcagagaagcagaacctcagcaggcacaggaagaagcaggtgctcagctcaatctgcatggtggcggcgtttaaaccaacctgaaaaaaagtgatttcaggcaggtgctccaggtaattaaacattaataccccaccaaccaaccatcccttaaacccttacctcttcaggagcttgtggatctgtgtgacggcttctcctggtgaaggggcttttataccccctccttccaacccaggctgctgatccctgccaagctgactccaaacctgctgattctgattattgacttagtcaacaaaaggagaataagtaacctacacaaatatgaaccttgcctagggagattagagtatcggaacactcgctctacgaaatgtgcagacagacggggatcctctagagctacctgctgatcgcccggcccctgttcaaacatgtcctaatactctgtctctgcaagggtcatcagtagttttccatcttactcaacatcctcccagtgggtaccgtggacttagcccctgtttgctcctccgataactggggtgaccttggttaatattcaccagcagcctcccccggtggacttagcccctgtttgctcctccgataactggggtgaccttggttaatattcaccagcagcctcccccggtggacttagcccctgtttgctcctccgataactggggtgaccttggttaatattcaccagcagcctcccccgcggccgcgataagtagcatggcgggttaatcattaactacaaggaacccctagtgatggagcctcagcttggccactccctctctgcgcgctcgctcgctcactgaggccgcccgggcaaagcccgggcgtcgggcgacctttggtcgcccggcctcant

SEQ ID NO: 43 - DNA, 3605 bp, synthetic construct
  source 1..3605: mol_type = other DNA
  misc_difference 57: note = a, c, t, g, unknown or other
ctgaggccgcccgggcaaagcccgggcgtcgggcgacctttggtcgcccggcctcantgagctagtgagctagcagagagggagtggccaagctgaggctccatcactaggggttccttgtagttaatgattaacccaccatgctacttatggctaggactagactagtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagtgacctctctgggcccatgccacctccaacatccacttgaccccttggaatttcagtggagaggagcagaggttgtcctggtgtggtttaggtagtgtgagagggtccaggttcaaaaccacttgctgggtggggagttgtcagtaagtggctatgccccaaccctgaagcctgtttccccatctgtacaatggaaatgataaagacccccatctgatagggtttttgtggcaaataaacatttggtttttttgttttgttttgttttgttttttgagatggaggtttgctctgtggcccaggctggagtgcagtgacacaatctcatctcaccacaaccttcccctgcctcatcctcccaagtagctgggattacaagcatgtgccaccacacctggctaattttctatttttagtagagatgggtttctccatgttggtcaggctcagcctcccaagtaactgggattacaggcctgtgccaccacacctggctaattttttctatttttgacagggatggggtttcaccatgttggtcaggctggtctagaggtactggatcttgctaccagtggaacagccactaaggattctgcagtgagagcagagggccagctaagtggtactctcccagagactgtctgactcatgccaccccctccaccttggacacaggacactgtggtttctgagccaggtacaatgactccttttggtaagtgcagtggaagctgtacactgcccaggcaaagtgtctgggcagcataggcaggtgactcagatcccagccagtggacttagcccctgtttgctcctctgataactggggtgaccttggttaatattcaccagcagcctcccctgttgcccctctggatccactgcttaaatacagacaaggacagggccctgtctcttcagcttcaggcaccaccactgacctgggacagtgaataattactctaaggtaaatataaaatttttaagtgtataatgtgttaaactactgattctaattgtttctctcttttagattccaacctttggaactgaaccatggaggatgccaagaacattaagaaaggccctgcccccttctaccccctggaggatggaacagcaggggagcagctgcacaaggccatgaagagatatgccctggtcccaggcaccattgccttcactgatgcccacattgaagttgatatcacctatgctgaatattttgagatgtctgtgagactggcagaggctatgaaaaggtatggactgaacaccaaccacagaattgtggtgtgcagtgaaaattccctgcagttcttcatgcctgtgcttggagctctcttcattggagtagctgttgctccagccaatgacatctacaatgaaagagagctcctcaactccatgggcatctcccagcccactgtggtgtttgtgtccaaaaaggggctgcagaaaattctgaatgtgcagaagaagctgccaatcatccagaagatcattatcatggacagcaagacagattaccagggtttccagagcatgtacacctttgtcaccagccacctccctcctggcttcaatgagtatgattttgttccagagagctttgacagagataaaacaattgcactgattatgaacagctctggcagcacaggtctgcccaaaggtgtggccttgccccacaggactgcctgtgtcaggttctctcatgccagggaccccatctttggaaaccagatcatccctgatacagccatcctgtctgttgtgcctttccatcatggctttggcatgttcaccaccctgggctacctgatctgtggattcagagtagtgctgatgtataggtttgaggaggaactgttcctgaggagccttcaggactacaagatccaatctgctctgctggtgcccaccctcttttccttctttgccaagagcaccttgattgataagtatgacctgagcaacctgcatgaaattgcttctggaggagcccctctgtccaaggaagtgggagaggcagtggccaaaagattccacctgcctggaatcagacagggctatggcctgacagagacaacttctgccattctcatcactccagaaggagatgacaagccaggagcagtgggcaaagtggtgccattttttgaagccaaggtggtggatctggacacaggcaagactctgggagtgaatcagagaggtgagctgtgtgtgaggggccccatgatcatgtcaggatatgtgaacaaccctgaggccaccaatgccctcattgacaaagatggctggctgcacagtggagacattgcctactgggatgaagatgagcacttcttcatagtggacaggctgaagtccctcatcaaatacaaagggtaccaagtggctcctgctgagctggagtccatcctgctccagcaccccaacatctttgatgcaggagtggcaggcctcccagatgatgatgctggagaacttccagctgctgtggttgtcctggaacatggaaagaccatgactgagaaggagattgttgactatgttgccagccaggtgaccacagctaagaagctcagaggaggggtggtctttgtagatgaggtgcccaagggcctgactggaaaactggatgccagaaaaatcagggagatcttgatcaaggcaaagaaaggagggaagattgctgtgtgattaattaagcttgctttcttgctgtccaatttctattaaaggttcctttgttccctaagtccaactactaaactgggggatattatgaagggccttgagcatctggattctgcctcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtacttaagccacaatctgcctcccagtagtacatgacattagtttattaatagcctaggaacccctagtgatggagttggccactccctctctgctagctcactagctcactgagggaggccgcccgggcaaagcccgggcgtcgggcgacctttggtcgcccggcctccctc

SEQ ID NO: 44 - DNA, 3606 bp, synthetic construct
  source 1..3606: mol_type = other DNA
  modified_base 3605: mod_base = OTHER; note = Inosine
gaggccgcccgggcaaagcccgggcgtcgggcgacctttggtcgcccggcctccctccctcagtgagctagtgagctagcagagagggagtggccaactccatcactaggggttcctaggctattaataaactaatgtcatgtactactgggaggcagattgtggcttaagtaccacatttgtagaggttttacttgctttaaaaaacctcccacatctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgaggcagaatccagatgctcaaggcccttcataatatcccccagtttagtagttggacttagggaacaaaggaacctttaatagaaattggacagcaagaaagcaagcttaattaatcacacagcaatcttccctcctttctttgccttgatcaagatctccctgatttttctggcatccagttttccagtcaggcccttgggcacctcatctacaaagaccacccctcctctgagcttcttagctgtggtcacctggctggcaacatagtcaacaatctccttctcagtcatggtctttccatgttccaggacaaccacagcagctggaagttctccagcatcatcatctgggaggcctgccactcctgcatcaaagatgttggggtgctggagcaggatggactccagctcagcaggagccacttggtaccctttgtatttgatgagggacttcagcctgtccactatgaagaagtgctcatcttcatcccagtaggcaatgtctccactgtgcagccagccatctttgtcaatgagggcattggtggcctcagggttgttcacatatcctgacatgatcatggggcccctcacacacagctcacctctctgattcactcccagagtcttgcctgtgtccagatccaccaccttggcttcaaaaaatggcaccactttgcccactgctcctggcttgtcatctccttctggagtgatgagaatggcagaagttgtctctgtcaggccatagccctgtctgattccaggcaggtggaatcttttggccactgcctctcccacttccttggacagaggggctcctccagaagcaatttcatgcaggttgctcaggtcatacttatcaatcaaggtgctcttggcaaagaaggaaaagagggtgggcaccagcagagcagattggatcttgtagtcctgaaggctcctcaggaacagttcctcctcaaacctatacatcagcactactctgaatccacagatcaggtagcccagggtggtgaacatgccaaagccatgatggaaaggcacaacagacaggatggctgtatcagggatgatctggtttccaaagatggggtccctggcatgagagaacctgacacaggcagtcctgtggggcaaggccacacctttgggcagacctgtgctgccagagctgttcataatcagtgcaattgttttatctctgtcaaagctctctggaacaaaatcatactcattgaagccaggagggaggtggctggtgacaaaggtgtacatgctctggaaaccctggtaatctgtcttgctgtccatgataatgatcttctggatgattggcagcttcttctgcacattcagaattttctgcagcccctttttggacacaaacaccacagtgggctgggagatgcccatggagttgaggagctctctttcattgtagatgtcattggctggagcaacagctactccaatgaagagagctccaagcacaggcatgaagaactgcagggaattttcactgcacaccacaattctgtggttggtgttcagtccataccttttcatagcctctgccagtctcacagacatctcaaaatattcagcataggtgatatcaacttcaatgtgggcatcagtgaaggcaatggtgcctgggaccagggcatatctcttcatggccttgtgcagctgctcccctgctgttccatcctccagggggtagaagggggcagggcctttcttaatgttcttggcatcctccatggttcagttccaaaggttggaatctaaaagagagaaacaattagaatcagtagtttaacacattatacacttaaaaattttatatttaccttagagtaattattcactgtcccaggtcagtggtggtgcctgaagctgaagagacagggccctgtccttgtctgtatttaagcagtggatccagaggggcaacaggggaggctgctggtgaatattaaccaaggtcaccccagttatcagaggagcaaacaggggctaagtccactggctgggatctgagtcacctgcctatgctgcccagacactttgcctgggcagtgtacagcttccactgcacttaccaaaaggagtcattgtacctggctcagaaaccacagtgtcctgtgtccaaggtggagggggtggcatgagtcagacagtctctgggagagtaccacttagctggccctctgctctcactgcagaatccttagtggctgttccactggtagcaagatccagtacctctagaccagcctgaccaacatggtgaaaccccatccctgtcaaaaatagaaaaaattagccaggtgtggtggcacaggcctgtaatcccagttacttgggaggctgagcctgaccaacatggagaaacccatctctactaaaaatagaaaattagccaggtgtggtggcacatgcttgtaatcccagctacttgggaggatgaggcaggggaaggttgtggtgagatgagattgtgtcactgcactccagcctgggccacagagcaaacctccatctcaaaaaacaaaacaaaacaaaacaaaaaaaccaaatgtttatttgccacaaaaaccctatcagatgggggtctttatcatttccattgtacagatggggaaacaggcttcagggttggggcatagccacttactgacaactccccacccagcaagtggttttgaacctggaccctctcacactacctaaaccacaccaggacaacctctgctcctctccactgaaattccaaggggtcaagtggatgttggaggtggcatgggcccagagaggtcactgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagctgctggaggatgggaactgaggggttggaagggggcagggtgagcccagaaactcctgtgtgcctctgagcctactagtctagtcctagccataagtagcatggtgggttaatcattaactacaaggaacccctagtgatggagcctcagcttggccactccctctctgctagctcactagctcactgaggccgcccgggcaaagcccgggcgtcgggcgacctttggtcgcccggcctcant

SEQ ID NO: 45 - DNA, 4647 bp, synthetic construct
  source 1..4647: mol_type = other DNA
  misc_difference 57: note = a, c, t, g, unknown or other
ctgaggccgcccgggcaaagcccgggcgtcgggcgacctttggtcgcccggcctcantgagcgagcgagcgcgcagagagggagtggccaagctgaggctccatcactaggggttccttgtagttaatgattaacccgccatgctacttatcgcggccgcgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccacggtacccactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgaagaggtaagggtttaagggatggttggttggtggggtattaatgtttaattacctggagcacctgcctgaaatcactttttttcaggttgggtttaaacgccgccaccatgcagagggtgaacatgatcatggctgagagccctggcctgatcaccatctgcctgctgggctacctgctgtctgctgaatgtacaggtttgtttccttttttataatacattgagtatgcttgccttttagatatagaaatatctgattctgtcttcttcactaaattttgattacatgatttgacagcaatattgaagagtctaacagccagcacccaggttggtaagtactggttctttgttagctaggttttcttcttcttcacttttaaaactaaatagatggacaatgcttatgatgcaataaggtttaataaacactgttcagttcagtatttggtcatgtaattcctgttaaaaaacagtcatctccttggtttaaaaaaattaaaagtgggaaaacaaagaaatagcagaatatagtgaaaaaaaataaccacagtatttttgtttggacttaccactttgaaatcaaattgggaaacaaaagcacaaacagtggccttatttacacaaaaagtctgattttaagatatgtgacaattcaaggtttcagaagtatgtaaggaggtgtgtctctaattttttaaattatatatcttcaatttaaagttttagttaaaacataaagattaacctttcattagcaagctgttagttatcaccaaagcttttcatggattaggaaaaaatcattttgtctctatctcaaacatcttggagttgatatttggggaaacacaatactcagttgagttccctaggggagaaaagcaagcttaagaattgacacaaagagtaggaagttagctattgcaacatatatcactttgttttttcacaactacagtgactttatttatttcccagaggaaggcatacagggaagaaattatcccatttggacaaacagcatgttctcacagtaagcacttatcacacttacttgtcaactttctagaatcaaatctagtagctgacagtaccaggatcaggggtgccaaccctaagcacccccagaaagctgactggccctgtggttcccactccagacatgatgtcagctgtgaaatccacctccctggaccataattaggcttctgttcttcaggagacatttgttcaaagtcatttgggcaaccatattctgaaaacagcccagccagggtgatggatcactttgcaaagatcctcaatgagctattttcaagtgatgacaaagtgtgaagttaagggctcatttgagaactttctttttcatccaaagtaaattcaaatatgattagaaatctgaccttttattactggaattctcttgactaaaagtaaaattgaattttaattcctaaatctccatgtgtatacagtactgtgggaacatcacagattttggctccatgccctaaagagaaattggctttcagattatttggattaaaaacaaagactttcttaagagatgtaaaattttcatgatgttttcttttttgctaaaactaaagaattattcttttacatttcagtttttcttgatcatgaaaatgccaacaaaattctgaatagaccaaagaggtataactctggcaagcttgaagagtttgtacaggggaatctggagagagagtgtatggaagagaagtgcagctttgaggaagccagagaagtgtttgaaaatacagagagaacaactgaattttggaagcagtatgtggatggtgatcaatgtgagagcaatccctgcttgaatggggggagctgtaaagatgatatcaacagctatgaatgttggtgtccctttggatttgaggggaaaaactgtgagcttgatgtgacctgtaatatcaagaatggcaggtgtgagcaattttgcaagaattctgctgataacaaagtggtctgtagctgcactgagggatataggctggctgaaaaccagaagagctgtgaacctgcagtgccttttccctgtgggagagtgtctgtgagccaaaccagcaagctgactagggctgaagcagtctttcctgatgtagattatgtgaatagcactgaggctgagacaatccttgacaatatcactcagagcacacagagcttcaatgacttcaccagggtggtaggaggggaggatgccaagcctgggcagttcccctggcaggtagtgctcaatggaaaagtggatgccttttgtggaggttcaattgtaaatgagaagtggattgtgactgcagcccactgtgtggaaactggagtcaagattactgtggtggctggagagcacaatattgaggaaactgagcacactgagcagaagaggaatgtgatcaggattatcccccaccacaactacaatgctgctatcaacaagtacaaccatgacattgccctcctggaactggatgaacccctggtcttgaacagctatgtgacacccatctgtattgctgataaagagtacaccaacatcttcttgaaatttgggtctggatatgtgtctggctggggcagggtgttccataaaggcaggtctgccctggtattgcagtatttgagggtgcctctggtggatagagcaacctgcttgctgagcaccaagtttacaatctacaacaatatgttctgtgcagggttccatgaaggtggtagagacagctgccagggagattctgggggtccccatgtgactgaggtggagggaaccagcttcctgactgggattatcagctggggtgaggagtgtgctatgaagggaaagtatgggatctacacaaaagtatccagatatgtgaactggattaaggagaaaaccaagctgacttgattaattaagagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctctagagcatggctacgtagataagtagcatggcgggttaatcattaactacacctgcaggaggaacccctagtgatggagttggccactccctctctgcgcgctcgctcgctcactgagggaggccgcccgggcaaagcccgggcgtcgggcgacctttggtcgcccggcctccctc

SEQ ID NO: 46 - DNA, 4648 bp, synthetic construct
  source 1..4648: mol_type = other DNA
  modified_base 4647: mod_base = OTHER; note = Inosine
gaggccgcccgggcaaagcccgggcgtcgggcgacctttggtcgcccggcctccctccctcagtgagcgagcgagcgcgcagagagggagtggccaactccatcactaggggttcctcctgcaggtgtagttaatgattaacccgccatgctacttatctacgtagccatgctctagagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagacaacaccacggaattatcagtgcccagcaacctagcccctgtccagcagcgggcaaggcaggcggcgatgagttctgccgtggcgatcgggagggggaaagcgaaagtcccagaaaggagttgacaggtggtggcaatgccccagccagtgggggttgcgtcagcaaacacagagcacaccacgccacgttgacggacaacgggccacaactcctctaaaagagacagcaaccaggatttatacaaggaggagaaaacgaaagccgtacgggaagcaatagctagatacagaggctataaagcagcatatccacacagcgtaaaaggagcaacatagttaagaatatcagtcaatctttcacaaattttgtaatccagaggttgattaacaggaacagagcgtaaataacgggaaagtttcttaacatgtttgtcttgtggcaatacacctgaactagtaattacatatccctaaaaatgtaaatgattgccccaccattttgttttattaacatttaaatgtatacccaaatcaagaaaaacagaacaaatatgggaataaatggcggtaagatgctcttaattaatcaagtcagcttggttttctccttaatccagttcacatatctggatacttttgtgtagatcccatactttcccttcatagcacactcctcaccccagctgataatcccagtcaggaagctggttccctccacctcagtcacatggggacccccagaatctccctggcagctgtctctaccaccttcatggaaccctgcacagaacatattgttgtagattgtaaacttggtgctcagcaagcaggttgctctatccaccagaggcaccctcaaatactgcaataccagggcagacctgcctttatggaacaccctgccccagccagacacatatccagacccaaatttcaagaagatgttggtgtactctttatcagcaatacagatgggtgtcacatagctgttcaagaccaggggttcatccagttccaggagggcaatgtcatggttgtacttgttgatagcagcattgtagttgtggtgggggataatcctgatcacattcctcttctgctcagtgtgctcagtttcctcaatattgtgctctccagccaccacagtaatcttgactccagtttccacacagtgggctgcagtcacaatccacttctcatttacaattgaacctccacaaaaggcatccacttttccattgagcactacctgccaggggaactgcccaggcttggcatcctcccctcctaccaccctggtgaagtcattgaagctctgtgtgctctgagtgatattgtcaaggattgtctcagcctcagtgctattcacataatctacatcaggaaagactgcttcagccctagtcagcttgctggtttggctcacagacactctcccacagggaaaaggcactgcaggttcacagctcttctggttttcagccagcctatatccctcagtgcagctacagaccactttgttatcagcagaattcttgcaaaattgctcacacctgccattcttgatattacaggtcacatcaagctcacagtttttcccctcaaatccaaagggacaccaacattcatagctgttgatatcatctttacagctccccccattcaagcagggattgctctcacattgatcaccatccacatactgcttccaaaattcagttgttctctctgtattttcaaacacttctctggcttcctcaaagctgcacttctcttccatacactctctctccagattcccctgtacaaactcttcaagcttgccagagttatacctctttggtctattcagaattttgttggcattttcatgatcaagaaaaactgaaatgtaaaagaataattctttagttttagcaaaaaagaaaacatcatgaaaattttacatctcttaagaaagtctttgtttttaatccaaataatctgaaagccaatttctctttagggcatggagccaaaatctgtgatgttcccacagtactgtatacacatggagatttaggaattaaaattcaattttacttttagtcaagagaattccagtaataaaaggtcagatttctaatcatatttgaatttactttggatgaaaaagaaagttctcaaatgagcccttaacttcacactttgtcatcacttgaaaatagctcattgaggatctttgcaaagtgatccatcaccctggctgggctgttttcagaatatggttgcccaaatgactttgaacaaatgtctcctgaagaacagaagcctaattatggtccagggaggtggatttcacagctgacatcatgtctggagtgggaaccacagggccagtcagctttctgggggtgcttagggttggcacccctgatcctggtactgtcagctactagatttgattctagaaagttgacaagtaagtgtgataagtgcttactgtgagaacatgctgtttgtccaaatgggataatttcttccctgtatgccttcctctgggaaataaataaagtcactgtagttgtgaaaaaacaaagtgatatatgttgcaatagctaacttcctactctttgtgtcaattcttaagcttgcttttctcccctagggaactcaactgagtattgtgtttccccaaatatcaactccaagatgtttgagatagagacaaaatgattttttcctaatccatgaaaagctttggtgataactaacagcttgctaatgaaaggttaatctttatgttttaactaaaactttaaattgaagatatataatttaaaaaattagagacacacctccttacatacttctgaaaccttgaattgtcacatatcttaaaatcagactttttgtgtaaataaggccactgtttgtgcttttgtttcccaatttgatttcaaagtggtaagtccaaacaaaaatactgtggttattttttttcactatattctgctatttctttgttttcccacttttaatttttttaaaccaaggagatgactgttttttaacaggaattacatgaccaaatactgaactgaacagtgtttattaaaccttattgcatcataagcattgtccatctatttagttttaaaagtgaagaagaagaaaacctagctaacaaagaaccagtacttaccaacctgggtgctggctgttagactcttcaatattgctgtcaaatcatgtaatcaaaatttagtgaagaagacagaatcagatatttctatatctaaaaggcaagcatactcaatgtattataaaaaaggaaacaaacctgtacattcagcagacagcaggtagcccagcaggcagatggtgatcaggccagggctctcagccatgatcatgttcaccctctgcatggtggcggcgtttaaacccaacctgaaaaaaagtgatttcaggcaggtgctccaggtaattaaacattaataccccaccaaccaaccatcccttaaacccttacctcttcaggagcttgtggatctgtgtgacggcttctcctggtgaaggggcttttataccccctccttccaacccaggctgctgatccctgccaagctgactccaaacctgctgattctgattattgacttagtcaacaaaaggagaataagtaacctacacaaatatgaaccttgcctagggagattagagtatcggaacactcgctctacgaaatgtgcagacagacggggatcctctagagctacctgctgatcgcccggcccctgttcaaacatgtcctaatactctgtctctgcaagggtcatcagtagttttccatcttactcaacatcctcccagtgggtaccgtggacttagcccctgtttgctcctccgataactggggtgaccttggttaatattcaccagcagcctcccccggtggacttagcccctgtttgctcctccgataactggggtgaccttggttaatattcaccagcagcctcccccggtggacttagcccctgtttgctcctccgataactggggtgaccttggttaatattcaccagcagcctcccccgcggccgcgataagtagcatggcgggttaatcattaactacaaggaacccctagtgatggagcctcagcttggccactccctctctgcgcgctcgctcgctcactgaggccgcccgggcaaagcccgggcgtcgggcgacctttggtcgcccggcctcant

SEQ ID NO: 47 - DNA, 3605 bp, synthetic construct
  source 1..3605: mol_type = other DNA
  misc_difference 57: note = a, c, t, g, unknown or other
ctgaggccgcccgggcaaagcccgggcgtcgggcgacctttggtcgcccggcctcantgagcgagcgagcgcgcagagagggagtggccaagctgaggctccatcactaggggttccttgtagttaatgattaacccaccatgctacttatggctaggactagactagtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagtgacctctctgggcccatgccacctccaacatccacttgaccccttggaatttcagtggagaggagcagaggttgtcctggtgtggtttaggtagtgtgagagggtccaggttcaaaaccacttgctgggtggggagttgtcagtaagtggctatgccccaaccctgaagcctgtttccccatctgtacaatggaaatgataaagacccccatctgatagggtttttgtggcaaataaacatttggtttttttgttttgttttgttttgttttttgagatggaggtttgctctgtggcccaggctggagtgcagtgacacaatctcatctcaccacaaccttcccctgcctcatcctcccaagtagctgggattacaagcatgtgccaccacacctggctaattttctatttttagtagagatgggtttctccatgttggtcaggctcagcctcccaagtaactgggattacaggcctgtgccaccacacctggctaattttttctatttttgacagggatggggtttcaccatgttggtcaggctggtctagaggtactggatcttgctaccagtggaacagccactaaggattctgcagtgagagcagagggccagctaagtggtactctcccagagactgtctgactcatgccaccccctccaccttggacacaggacactgtggtttctgagccaggtacaatgactccttttggtaagtgcagtggaagctgtacactgcccaggcaaagtgtctgggcagcataggcaggtgactcagatcccagccagtggacttagcccctgtttgctcctctgataactggggtgaccttggttaatattcaccagcagcctcccctgttgcccctctggatccactgcttaaatacagacaaggacagggccctgtctcttcagcttcaggcaccaccactgacctgggacagtgaataattactctaaggtaaatataaaatttttaagtgtataatgtgttaaactactgattctaattgtttctctcttttagattccaacctttggaactgaaccatggaggatgccaagaacattaagaaaggccctgcccccttctaccccctggaggatggaacagcaggggagcagctgcacaaggccatgaagagatatgccctggtcccaggcaccattgccttcactgatgcccacattgaagttgatatcacctatgctgaatattttgagatgtctgtgagactggcagaggctatgaaaaggtatggactgaacaccaaccacagaattgtggtgtgcagtgaaaattccctgcagttcttcatgcctgtgcttggagctctcttcattggagtagctgttgctccagccaatgacatctacaatgaaagagagctcctcaactccatgggcatctcccagcccactgtggtgtttgtgtccaaaaaggggctgcagaaaattctgaatgtgcagaagaagctgccaatcatccagaagatcattatcatggacagcaagacagattaccagggtttccagagcatgtacacctttgtcaccagccacctccctcctggcttcaatgagtatgattttgttccagagagctttgacagagataaaacaattgcactgattatgaacagctctggcagcacaggtctgcccaaaggtgtggccttgccccacaggactgcctgtgtcaggttctctcatgccagggaccccatctttggaaaccagatcatccctgatacagccatcctgtctgttgtgcctttccatcatggctttggcatgttcaccaccctgggctacctgatctgtggattcagagtagtgctgatgtataggtttgaggaggaactgttcctgaggagccttcaggactacaagatccaatctgctctgctggtgcccaccctcttttccttctttgccaagagcaccttgattgataagtatgacctgagcaacctgcatgaaattgcttctggaggagcccctctgtccaaggaagtgggagaggcagtggccaaaagattccacctgcctggaatcagacagggctatggcctgacagagacaacttctgccattctcatcactccagaaggagatgacaagccaggagcagtgggcaaagtggtgccattttttgaagccaaggtggtggatctggacacaggcaagactctgggagtgaatcagagaggtgagctgtgtgtgaggggccccatgatcatgtcaggatatgtgaacaaccctgaggccaccaatgccctcattgacaaagatggctggctgcacagtggagacattgcctactgggatgaagatgagcacttcttcatagtggacaggctgaagtccctcatcaaatacaaagggtaccaagtggctcctgctgagctggagtccatcctgctccagcaccccaacatctttgatgcaggagtggcaggcctcccagatgatgatgctggagaacttccagctgctgtggttgtcctggaacatggaaagaccatgactgagaaggagattgttgactatgttgccagccaggtgaccacagctaagaagctcagaggaggggtggtctttgtagatgaggtgcccaagggcctgactggaaaactggatgccagaaaaatcagggagatcttgatcaaggcaaagaaaggagggaagattgctgtgtgattaattaagcttgctttcttgctgtccaatttctattaaaggttcctttgttccctaagtccaactactaaactgggggatattatgaagggccttgagcatctggattctgcctcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtacttaagccacaatctgcctcccagtagtacatgacattagtttattaatagcctaggaacccctagtgatggagttggccactccctctctgcgcgctcgctcgctcactgagggaggccgcccgggcaaagcccgggcgtcgggcgacctttggtcgcccggcctccctc

SEQ ID NO: 48 - DNA, 3606 bp, synthetic construct
  source 1..3606: mol_type = other DNA
  modified_base 3605: mod_base = OTHER; note = Inosine
gaggccgcccgggcaaagcccgggcgtcgggcgacctttggtcgcccggcctccctccctcagtgagcgagcgagcgcgcagagagggagtggccaactccatcactaggggttcctaggctattaataaactaatgtcatgtactactgggaggcagattgtggcttaagtaccacatttgtagaggttttacttgctttaaaaaacctcccacatctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgaggcagaatccagatgctcaaggcccttcataatatcccccagtttagtagttggacttagggaacaaaggaacctttaatagaaattggacagcaagaaagcaagcttaattaatcacacagcaatcttccctcctttctttgccttgatcaagatctccctgatttttctggcatccagttttccagtcaggcccttgggcacctcatctacaaagaccacccctcctctgagcttcttagctgtggtcacctggctggcaacatagtcaacaatctccttctcagtcatggtctttccatgttccaggacaaccacagcagctggaagttctccagcatcatcatctgggaggcctgccactcctgcatcaaagatgttggggtgctggagcaggatggactccagctcagcaggagccacttggtaccctttgtatttgatgagggacttcagcctgtccactatgaagaagtgctcatcttcatcccagtaggcaatgtctccactgtgcagccagccatctttgtcaatgagggcattggtggcctcagggttgttcacatatcctgacatgatcatggggcccctcacacacagctcacctctctgattcactcccagagtcttgcctgtgtccagatccaccaccttggcttcaaaaaatggcaccactttgcccactgctcctggcttgtcatctccttctggagtgatgagaatggcagaagttgtctctgtcaggccatagccctgtctgattccaggcaggtggaatcttttggccactgcctctcccacttccttggacagaggggctcctccagaagcaatttcatgcaggttgctcaggtcatacttatcaatcaaggtgctcttggcaaagaaggaaaagagggtgggcaccagcagagcagattggatcttgtagtcctgaaggctcctcaggaacagttcctcctcaaacctatacatcagcactactctgaatccacagatcaggtagcccagggtggtgaacatgccaaagccatgatggaaaggcacaacagacaggatggctgtatcagggatgatctggtttccaaagatggggtccctggcatgagagaacctgacacaggcagtcctgtggggcaaggccacacctttgggcagacctgtgctgccagagctgttcataatcagtgcaattgttttatctctgtcaaagctctctggaacaaaatcatactcattgaagccaggagggaggtggctggtgacaaaggtgtacatgctctggaaaccctggtaatctgtcttgctgtccatgataatgatcttctggatgattggcagcttcttctgcacattcagaattttctgcagcccctttttggacacaaacaccacagtgggctgggagatgcccatggagttgaggagctctctttcattgtagatgtcattggctggagcaacagctactccaatgaagagagctccaagcacaggcatgaagaactgcagggaattttcactgcacaccacaattctgtggttggtgttcagtccataccttttcatagcctctgccagtctcacagacatctcaaaatattcagcataggtgatatcaacttcaatgtgggcatcagtgaaggcaatggtgcctgggaccagggcatatctcttcatggccttgtgcagctgctcccctgctgttccatcctccagggggtagaagggggcagggcctttcttaatgttcttggcatcctccatggttcagttccaaaggttggaatctaaaagagagaaacaattagaatcagtagtttaacacattatacacttaaaaattttatatttaccttagagtaattattcactgtcccaggtcagtggtggtgcctgaagctgaagagacagggccctgtccttgtctgtatttaagcagtggatccagaggggcaacaggggaggctgctggtgaatattaaccaaggtcaccccagttatcagaggagcaaacaggggctaagtccactggctgggatctgagtcacctgcctatgctgcccagacactttgcctgggcagtgtacagcttccactgcacttaccaaaaggagtcattgtacctggctcagaaaccacagtgtcctgtgtccaaggtggagggggtggcatgagtcagacagtctctgggagagtaccacttagctggccctctgctctcactgcagaatccttagtggctgttccactggtagcaagatccagtacctctagaccagcctgaccaacatggtgaaaccccatccctgtcaaaaatagaaaaaattagccaggtgtggtggcacaggcctgtaatcccagttacttgggaggctgagcctgaccaacatggagaaacccatctctactaaaaatagaaaattagccaggtgtggtggcacatgcttgtaatcccagctacttgggaggatgaggcaggggaaggttgtggtgagatgagattgtgtcactgcactccagcctgggccacagagcaaacctccatctcaaaaaacaaaacaaaacaaaacaaaaaaaccaaatgtttatttgccacaaaaaccctatcagatgggggtctttatcatttccattgtacagatggggaaacaggcttcagggttggggcatagccacttactgacaactccccacccagcaagtggttttgaacctggaccctctcacactacctaaaccacaccaggacaacctctgctcctctccactgaaattccaaggggtcaagtggatgttggaggtggcatgggcccagagaggtcactgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagctgctggaggatgggaactgaggggttggaagggggcagggtgagcccagaaactcctgtgtgcctctgagcctactagtctagtcctagccataagtagcatggtgggttaatcattaactacaaggaacccctagtgatggagcctcagcttggccactccctctctgcgcgctcgctcgctcactgaggccgcccgggcaaagcccgggcgtcgggcgacctttggtcgcccggcctcant

SEQ ID NO: 49 - DNA, 154 bp, synthetic construct
  source 1..154: mol_type = other DNA
  modified_base 62: mod_base = OTHER; note = Nucleotide with phosphorothioate linkage
  modified_base 63: mod_base = OTHER; note = Nucleotide with phosphorothioate linkage
  modified_base 64: mod_base = OTHER; note = Nucleotide with phosphorothioate linkage
  modified_base 65: mod_base = OTHER; note = Nucleotide with phosphorothioate linkage
  modified_base 66: mod_base = OTHER; note = Nucleotide with phosphorothioate linkage
aggaacccctagtgatggagcctcagcttggccactccctctctgctagctcactagctcactgaggccgcccgggcaaagcccgggcgtcgggcgacctttggtcgcccggcctcagtgagctagtgagctagcagagagggagtggccaagc

SEQ ID NO: 50 - DNA, 57 bp, synthetic construct
  source 1..57: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  modified_base 4: mod_base = OTHER; note = Nucleotide with phosphorothioate linkage
  modified_base 5: mod_base = OTHER; note = Nucleotide with phosphorothioate linkage
ctgaggccgcccgggcaaagcccgggcgtcgggcgacctttggtcgcccggcctcag

SEQ ID NO: 51 - DNA, 154 bp, synthetic construct; identical to SEQ ID NO: 49
  source 1..154: mol_type = other DNA
  modified_base 113: mod_base = OTHER; note = Nucleotide with phosphorothioate linkage
  modified_base 114: mod_base = OTHER; note = Nucleotide with phosphorothioate linkage
  modified_base 115: mod_base = OTHER; note = Nucleotide with phosphorothioate linkage
  modified_base 116: mod_base = OTHER; note = Nucleotide with phosphorothioate linkage
  modified_base 117: mod_base = OTHER; note = Nucleotide with phosphorothioate linkage

SEQ ID NO: 52 - DNA, 57 bp, synthetic construct; identical to SEQ ID NO: 50
  source 1..57: mol_type = other DNA
  modified_base 3: mod_base = OTHER; note = Nucleotide with phosphorothioate linkage
  modified_base 4: mod_base = OTHER; note = Nucleotide with phosphorothioate linkage
  modified_base 5: mod_base = OTHER; note = Nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage

SEQ ID NO: 53 - DNA, 154 bp, synthetic construct; identical to SEQ ID NO: 49
  source 1..154: mol_type = other DNA

SEQ ID NO: 54 - DNA, 19 bp, synthetic construct
  source 1..19: mol_type = other DNA
ctgaggccgtcggcctcag

SEQ ID NO: 55 - DNA, 19 bp, synthetic construct
  source 1..19: mol_type = other DNA
  modified_base 3: mod_base = OTHER; note = Nucleotide with phosphorothioate linkage
  modified_base 4: mod_base = OTHER; note = Nucleotide with phosphorothioate linkage
  modified_base 5: mod_base = OTHER; note = Nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
ctgaggccgtcggcctcag

SEQ ID NO: 56 - DNA, 61 bp, synthetic construct
  source 1..61: mol_type = other DNA
  modified_base 61: mod_base = OTHER; note = Inosine
ctcactgaggccgcccgggcaaagcccgggcgtcgggcgacctttggtcgcccggcctcan

SEQ ID NO: 57 - DNA, 61 bp, synthetic construct
  source 1..61: mol_type = other DNA
  modified_base 60: mod_base = OTHER; note = Inosine
ctcactgaggccgcccgggcaaagcccgggcgtcgggcgacctttggtcgcccggcctcng

SEQ ID NO: 58 - DNA, 61 bp, synthetic construct
  source 1..61: mol_type = other DNA
  modified_base 57: mod_base = OTHER; note = Inosine
ctcactgaggccgcccgggcaaagcccgggcgtcgggcgacctttggtcgcccggcntcag

SEQ ID NO: 59 - DNA, 58 bp, synthetic construct
  source 1..58: mol_type = other DNA
  modified_base 57: mod_base = OTHER; note = Inosine
ctcactgaggccgcccgggcaaagcccgggcgtcgggcgacctttggtcgcccggcnt

SEQ ID NO: 60 - DNA, 56 bp, synthetic construct
  source 1..56: mol_type = other DNA
  modified_base 55: mod_base = OTHER; note = Inosine
ctcactgaggccgcccgggcaaagcccgggcgtcgggcgacctttggtcgcccgnc

SEQ ID NO: 61 - DNA, 194 bp, synthetic construct
  source 1..194: mol_type = other DNA
cactgtgctggagtcgacacgtaccaataaaaagacagaataaaactgggaaacccagctaccagtgaattctcctgagatggacgggcaactgcgtctcattcacgttagagactccaaccgtccatctcaggagaattcactggtagttttattctgtctttttattggtacgtgtcgactccagcacagtg

SEQ ID NO: 62 - DNA, 199 bp, synthetic construct
  source 1..199: mol_type = other DNA
gtgctggagtcgacacgtaccaataaaaagacagaataaaactgggaaacccagctaccagtgaattctcctgagatggacgggcaactgcgtctcattcacgttagagactccaaccgtccatctcaggagaattcactggtaggaccctttgggtcttttattctgtctttttattggtacgtgtcgactccagcac

SEQ ID NO: 63 - DNA, 194 bp, synthetic construct; identical to SEQ ID NO: 61
  source 1..194: mol_type = other DNA

SEQ ID NO: 64 - DNA, 200 bp, synthetic construct
  source 1..200: mol_type = other DNA
cactgtgctggagtcgacacgtaccaataaaaagacagaataaaacttcgggcaaagcccgaagctaccagtgaattctcctgagatggacgggcaactgcgtctcattcacgttagagactccaaccgtccatctcaggagaattcactggtagttttattctgtctttttattggtacgtgtcgactccagcacagtg

SEQ ID NO: 65 - DNA, 199 bp, synthetic construct
  source 1..199: mol_type = other DNA
gtgctggagtcgacacgtaccaataaaaagacagaataaaactgtgaaactcagctaccagtgaattctcctgagatggacgggcaactgcgtctcattcacgttagagactccaaccgtccatctcaggagaattcactggtagctgagaaacacagttttattctgtctttttattggtacgtgtcgactccagcac

SEQ ID NO: 66 - DNA, 199 bp, synthetic construct
  source 1..199: mol_type = other DNA
gtgctggagtcgacacgtaccaataaaaagacagaataaaaggctcacgtcgggctaccagtgaattctcctgagatggacgggcaactgcgtctcattcacgttagagactccaaccgtccatctcaggagaattcactggtagcggctgcactcgcttttattctgtctttttattggtacgtgtcgactccagcac

SEQ ID NO: 67 - DNA, 194 bp, synthetic construct
  source 1..194: mol_type = other DNA
cactgtgctggagtcgacacgtaccaataaaaagacagaataaaagttctaaatcattctaccagtgaattctcctgagatggacgggcaactgcgtctcattcacgttagagactccaaccgtccatctcaggagaattcactggtagttttattctgtctttttattggtacgtgtcgactccagcacagtg

SEQ ID NO: 68 - DNA, 199 bp, synthetic construct
  source 1..199: mol_type = other DNA
  modified_base 42: mod_base = OTHER; note = Nucleotide with phosphorothioate linkage
  modified_base 43: mod_base = OTHER; note = Nucleotide with phosphorothioate linkage
  modified_base 44: mod_base = OTHER; note = Nucleotide with phosphorothioate linkage
  modified_base 45: mod_base = OTHER; note = Nucleotide with phosphorothioate linkage
  modified_base 46: mod_base = OTHER; note = Nucleotide with phosphorothioate linkage
  modified_base 47: mod_base = OTHER; note = Nucleotide with phosphorothioate linkage
gtgctggagtcgacacgtaccaataaaaagacagaataaaactgtgaaactcagctaccagtgaattctcctgagatggacgggcaactgcgtctcattcacgttagagactccaaccgtccatctcaggagaattcactggtagctgagtttcacagttttattctgtctttttattggtacgtgtcgactccagcac

SEQ ID NO: 69 - DNA, 199 bp, synthetic construct; identical to SEQ ID NO: 68
  source 1..199: mol_type = other DNA